SEQUENCE LISTING (14 sequences)

SEQ ID NO: 1 - RNA, 18 nt, Homo sapiens
  source 1..18: mol_type = other RNA
tcctatgtaagattctgt

SEQ ID NO: 2 - DNA, 20 bp, synthetic construct
  source 1..20: mol_type = other DNA
gcagccttctggaaatatgg

SEQ ID NO: 3 - DNA, 27 bp, synthetic construct
  source 1..27: mol_type = other DNA
ttctctattcagaaatgaaagatcatt

SEQ ID NO: 4 - DNA, 20 bp, synthetic construct
  source 1..20: mol_type = other DNA
ctggacccgtcaagagagaa

SEQ ID NO: 5 - DNA, 23 bp, synthetic construct
  source 1..23: mol_type = other DNA
tgttcaacagtccagagtatcag

SEQ ID NO: 6 - DNA, 30 bp, synthetic construct
  source 1..30: mol_type = other DNA
aggctcaggatcgatcctataaatgaaaga

SEQ ID NO: 7 - DNA, 22 bp, synthetic construct
  source 1..22: mol_type = other DNA
acccgtcaagagagaattcaag

SEQ ID NO: 8 - DNA, 21 bp, synthetic construct
  source 1..21: mol_type = other DNA
gatgaggaggatttgcagagg

SEQ ID NO: 9 - DNA, 32 bp, synthetic construct
  source 1..32: mol_type = other DNA
atgtttctggaactaccttgcatacttagctg

SEQ ID NO: 10 - DNA, 24 bp, synthetic construct
  source 1..24: mol_type = other DNA
cctgatgtctgtgtcatatcttga

SEQ ID NO: 11 - DNA, 18 bp, synthetic construct
  source 1..18: mol_type = other DNA
tcggagagttctgggatt

SEQ ID NO: 12 - DNA, 27 bp, synthetic construct
  source 1..27: mol_type = other DNA
ccgcagctgcaaaatattgtatccaca

SEQ ID NO: 13 - DNA, 20 bp, synthetic construct
  source 1..20: mol_type = other DNA
aagtgcaatggtctttaggt

SEQ ID NO: 14 - DNA, 33 bp, synthetic construct
  source 1..33: mol_type = other DNA
gtgagcggctggcggctgttcaagaagattagc

(19) United States
(12) Patent Application Publication
(10) Pub. No.: US 2026/0001889 A1
(43) Pub. Date: Jan. 1, 2026

(54) COMPOSITIONS USEFUL FOR MODULATING SPLICING

(71) Applicant: Skyhawk Therapeutics, Inc., Waltham, MA (US)

(72) Inventors: Brian Lucas, Arlington, MA (US); Tiansheng Wang, Concord, MA (US); Hasane Ratni, Habsheim (FR); Sivaraman Dandapani, Wakefield, MA (US); Ingo Konetzki, Basel (CH); Michael Luzzio, Noank, CT (US)

(21) Appl. No.: 18/864,033

(22) PCT Filed: May 12, 2023
(86) PCT No.: PCT/US2023/022141

§ 371 Date: Nov. 8, 2024

Related U.S. Application Data
(60) Provisional application No. 63/341,358, filed on May 12, 2022.

(51) Int. Cl. C07D 491/048; A61K 31/4355; A61K 31/444; C07B 59/00

(52) U.S. Cl. CPC C07D 491/048; A61K 31/4355; A61K 31/444; C07B 59/002

(57) ABSTRACT

Described herein are compounds that modulate splicing of a pre-mRNA, encoded by genes, and methods of treating diseases and conditions associated with gene expression or activity of proteins encoded by genes.

### CROSS REFERENCE

This application claims the benefit of priority to U.S. Provisional Application No. 63/341,358, filed May 12, 2022, which is incorporated herein by reference in its entirety.

### BACKGROUND

Spinocerebellar Ataxia 3 (SCA3 or Machado-Joseph Disease) is a rare, inherited, neurodegenerative, autosomal dominant disease. It is characterized by progressive degeneration of the brainstem, cerebellum and spinal cord, however, neurons in other areas of the brain are also affected. Presenting features include gait problems, speech difficulties, clumsiness, and often visual blurring and diplopia; saccadic eye movements become slow and ophthalmoparesis develops, resulting initially in up-gaze restriction. Ambulation becomes increasingly difficult, leading to the need for assistive devices 10 to 15 years following onset. Late in the disease course, individuals are wheelchair bound and have severe dysarthria, dysphagia, facial and temporal atrophy. The disease progresses relentlessly until death occurs at any time from 6 to approximately 30 years after onset through pulmonary complications.

SCA3 is caused by CAG tri-nucleotide repeats in exon 10 of the Ataxin 3 (ATXN3) gene. ATXN3 encodes for a deubiquitinase with wide-ranging functions, but it does not appear to be an essential gene. Disease causing variants of the ATXN3 gene have approximately 40 to over 200 CAG tri-nucleotide repeats in exon 10. Expanded CAG repeats in the ATXN3 gene are translated into expanded polyglutamine repeats (polyQ) in the ataxin-3 protein and this toxic Ataxin 3 protein is associated with aggregates. The polyglutamine expanded ataxin-3 protein in these aggregates is ubiquitinated and the aggregates contain other proteins, including heat shock proteins and transcription factors. Aggregates are frequently observed in the brain tissue of SCA3 patients. There are currently no treatments for SCA3.

### SUMMARY

In one aspect, described herein is a compound of Formula (I), or a pharmaceutically acceptable salt thereof.

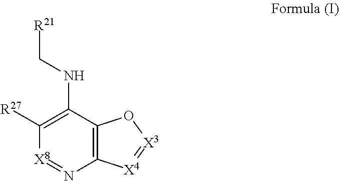

wherein X³, X⁴, X⁸, and R²¹ are as defined herein.

Also provided herein are pharmaceutical compositions comprising a compound disclosed herein, or a pharmaceutically acceptable salt thereof, and a pharmaceutically acceptable excipient or carrier.

In some aspects, described herein, is a method of modulating splicing of a Ataxin3 (ATXN3) pre-mRNA, comprising contacting a small molecule splicing modulator compound disclosed herein (SMSM) to the ATXN3 pre-mRNA with a splice site sequence or cells comprising the ATXN3 pre-mRNA, wherein the SMSM binds to the ATXN3 pre-mRNA and modulates splicing of the ATXN3 pre-mRNA in a cell of a subject to produce a spliced product of the ATXN3 pre-mRNA.

In some aspects, described herein, is a method of treating, preventing, delaying of progress, or ameliorating symptoms of a disease or a condition associated with Ataxin 3 (ATXN3) expression level or activity level in a subject in need thereof, comprising administering a therapeutically effective amount of a small molecule splicing modulator compound disclosed herein (SMSM), wherein the SMSM binds to a pre-mRNA encoded by ATXN3 and modulates splicing of the ATXN3 pre-mRNA in a cell of the subject to produce a spliced product of the ATXN3 pre-mRNA, wherein the amount of full length ATXN3 is reduced.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

The term “small molecule splicing modulator” or “SMSM” denotes a small molecule compound that binds to a cell component (e.g., DNA, RNA, pre-mRNA, protein, RNP, snRNA, carbohydrates, lipids, co-factors, nutrients, and/or metabolites) and modulates splicing. For example, a SMSM can bind to a polynucleotide, e.g., an RNA (e.g., a pre-mRNA) with an aberrant splice site, resulting in steric modulation of the polynucleotide. For example, a SMSM can bind to a protein, e.g., a spliceosome protein or a ribonuclear protein, resulting in steric modulation of the protein. For example, a SMSM can bind to a spliceosome component, e.g., a spliceosome protein or snRNA resulting in steric modulation of the spliceosome protein or snRNA. For example, a SMSM is a compound of Formula (I). The term “small molecule splicing modulator” or “SMSM” specifically excludes compounds consisting of oligonucleotides.

“Steric alteration,” “steric modification,” or “steric modulation” herein refers to changes in the spatial orientation of chemical moieties with respect to each other. A person of ordinary skill in the art would recognize steric mechanisms include, but are not limited to, steric hindrance, steric shielding, steric attraction, chain crossing, steric repulsions, steric inhibition of resonance, and steric inhibition of protonation.

Any open valency appearing on a carbon, oxygen, sulfur or nitrogen atom in the structures herein indicates the presence of a hydrogen, unless indicated otherwise.

The definitions described herein apply irrespective of whether the terms in question appear alone or in combination. It is contemplated that the definitions described herein can be appended to form chemically relevant combinations, such as e.g., “heterocycloalkylaryl,” “haloalkylheteroaryl,” “arylalkylheterocycloalkyl,” or “alkoxyalkyl.” The last member of the combination is the radical which is binding to the rest of the molecule. The other members of the combination are attached to the binding radical in reversed order in respect of the literal sequence, e.g., the combination arylalkylheterocycloalkyl refers to a heterocycloalkyl-radical which is substituted by an alkyl which is substituted by an aryl.

When indicating the number of substituents, the term “one or more” refers to the range from one substituent to the highest possible number of substitutions, i.e., replacement of one hydrogen up to replacement of all hydrogens by substituents.

The term “optional” or “optionally” denotes that a subsequently described event or circumstance can but need not occur, and that the description includes instances where the event or circumstance occurs and instances in which it does not.

The term “substituent” denotes an atom or a group of atoms replacing a hydrogen atom on the parent molecule.

The term “substituted” denotes that a specified group bears one or more substituents.

Where any group can carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitutions, i.e., replacement of one hydrogen up to replacement of all hydrogens by substituents.

The terms “compound(s) of this disclosure,” “compound(s) of the present disclosure,” “small molecule steric modulator,” “small molecule splicing modulator,” “steric modulator,” “splicing modulator,” “compounds that modify splicing,” and “compounds modifying splicing” are interchangeably used herein and refer to compounds as disclosed herein and stereoisomers, tautomers, solvates, and salts (e.g., pharmaceutically acceptable salts) thereof.

The following abbreviations are used throughout the specification: acetic acid (AcOH); ethyl acetate (EtOAc); butyl alcohol (n-BuOH); 1,2-dichloroethane (DCE); dichloromethane (CH₂Cl₂, DCM); diisopropylethylamine (Diipea); dimethylformamide (DMF); hydrogen chloride (HCl); methanol (MeOH); methoxymethyl bromide (MOMBr); N-methyl-2-pyrrolidone (NMP); methyl Iodide (Mel); n-propanol (n-PrOH); p-methoxybenzyl (PMB); triethylamine (Et₃N); [1,1′-Bis(diphenylphosphino)ferrocene]dichloropalladium(II); (Pd(dppf)Cl₂); sodium ethane thiolate (EtSNa); sodium acetate (NaOAc); sodium hydride (NaH); sodium hydroxide (NaOH); tetrahydropyran (THP); tetrahydrofuran (THF).

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “oxo” refers to the ═O substituent.

“Carboxyl” refers to —COOH.

“Cyano” refers to —CN.

The term “thioxo” refers to the ═S substituent.

The term “halo,” “halogen,” and “halide” are used interchangeably herein and denote fluoro, chloro, bromo, or iodo.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—.

In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkylamino” refers to a radical of the formula —NHR or —NRR where each R is, independently, an alkyl radical as defined above. Unless stated otherwise specifically in the specification, an alkylamino group may be optionally substituted as described below.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “aromatic” refers to a planar ring having a delocalized π-electron system containing 4n+2 π electrons, where n is an integer. Aromatics can be optionally substituted.

The term “aromatic” includes both aryl groups (e.g., phenyl, naphthalenyl) and heteroaryl groups (e.g., pyridinyl, furanyl, quinolinyl).

The term “aryl” refers to a radical derived from a hydrocarbon ring system comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example, 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Cyanoalkyl” refers to an alkyl radical, as defined above, that is substituted by one or more cyano groups. In some embodiments, the alkyl is substituted with one cyano group. In some embodiments, the alkyl is substituted with one, two, or three cyano groups. Aminoalkyl include, for example, cyanomethyl, cyanoethyl, cyanopropyl, cyanobutyl, or cyanopentyl.

The term “haloalkoxy” denotes an alkoxy group wherein at least one of the hydrogen atoms of the alkoxy group has been replaced by same or different halogen atoms, particularly fluoro atoms. Examples of haloalkoxyl include monofluoro-, difluoro- or trifluoro-methoxy, -ethoxy or -propoxy, for example, 3,3,3-trifluoropropoxy, 2-fluoroethoxy, 2,2,2-trifluoroethoxy, fluoromethoxy, or trifluoromethoxy. The term “perhaloalkoxy” denotes an alkoxy group where all hydrogen atoms of the alkoxy group have been replaced by the same or different halogen atoms.

The term “bicyclic ring system” denotes two rings which are fused to each other via a common single or double bond (annelated bicyclic ring system), via a sequence of three or more common atoms (bridged bicyclic ring system) or via a common single atom (spiro bicyclic ring system). Bicyclic ring systems can be saturated, partially unsaturated, unsaturated, or aromatic. Bicyclic ring systems can comprise heteroatoms selected from N, O, and S.

The terms “carbocyclic” or “carbocycle” refer to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

The term “cycloalkyl” refers to a monocyclic or polycyclic non-aromatic radical, wherein each of the atoms forming the ring (i.e., skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms.

Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1 (2H)-one, spiro[2.2]pentyl, norbornyl and bicycle[1.1.1]pentyl.

Unless otherwise stated specifically in the specification, a cycloalkyl group may be optionally substituted.

The term “bridged” refers to any ring structure with two or more rings that contains a bridge connecting two bridgehead atoms. The bridgehead atoms are defined as atoms that are the part of the skeletal framework of the molecule and which are bonded to three or more other skeletal atoms. In some embodiments, the bridgehead atoms are C, N, or P. In some embodiments, the bridge is a single atom or a chain of atoms that connects two bridgehead atoms. In some embodiments, the bridge is a valence bond that connects two bridgehead atoms. In some embodiments, the bridged ring system is cycloalkyl. In some embodiments, the bridged ring system is heterocycloalkyl.

The term “fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with one or more N, S, and O atoms. The non-limiting examples of fused heterocyclyl or heteroaryl ring structures include 6-5 fused heterocycle, 6-6 fused heterocycle, 5-6 fused heterocycle, 5-5 fused heterocycle, 7-5 fused heterocycle, and 5-7 fused heterocycle.

The term “haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

The term “haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

The term “fluoroalkyl” refers to an alkyl in which one or more hydrogen atoms are replaced by a fluorine atom. In one aspect, a fluoroalkyl is a C₁-C₆ fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, or —N(aryl)-), sulfur (e.g., —S—, —S(═O)—, or —S(═O)₂—), or combinations thereof.

In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂.

“Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon, or sulfur atoms in the heterocyclyl radical may be optionally oxidized. The nitrogen atom may be optionally quaternized. The heterocycloalkyl radical is partially or fully saturated.

Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides, and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d]oxazol-2 (3H)-onyl, 1H-benzo[d]imidazol-2 (3H)-onyl, benzo[d]thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” refers to an aryl group that includes one or more ring heteroatoms selected from nitrogen, oxygen, and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, oxo, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, oxo, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂(C₁-C₄ alkyl). In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “tautomer” refers to a proton shift from one atom of a molecule to another atom of the same molecule. The compounds presented herein may exist as tautomers.

Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

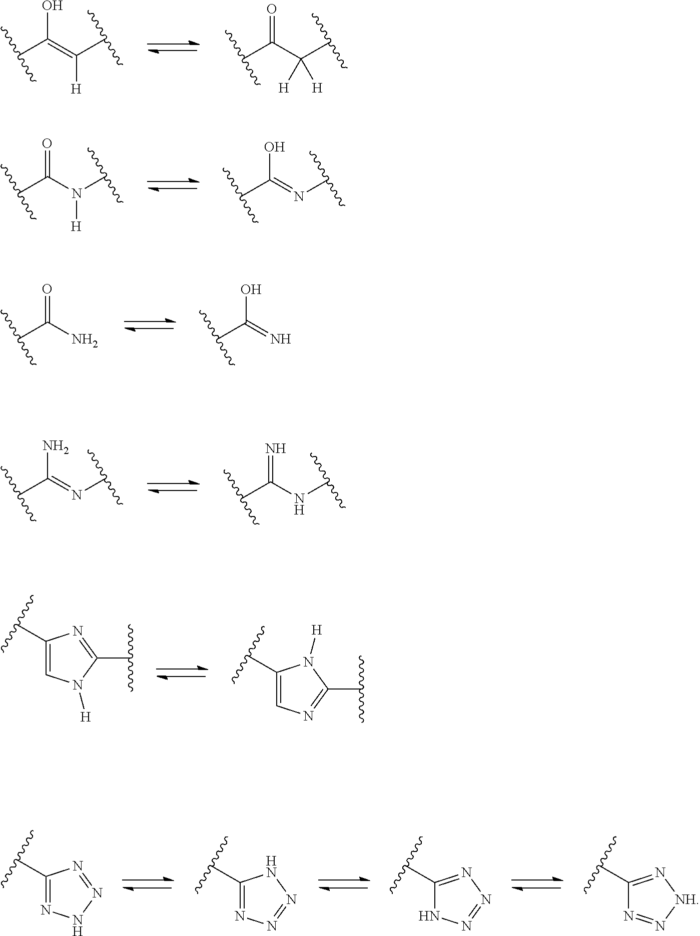

The terms “administer,” “administering,” “administration,” and the like, as used herein, refer to the methods that may be used to enable delivery of compounds or compositions to the desired site of biological action. These methods include but are not limited to oral routes (p.o.), intraduodenal routes (i.d.), parenteral injection (including intravenous (i.v.), subcutaneous (s.c.), intraperitoneal (i.p.), intramuscular (i.m.), intravascular or infusion (inf.)), topical (top.) and rectal (p.r.) administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a non-human animal is a mouse.

The term “pharmaceutically acceptable” denotes an attribute of a material which is useful in preparing a pharmaceutical composition that is generally safe, non toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

The term “pharmaceutically acceptable salts” denotes salts which are not biologically or otherwise undesirable. Pharmaceutically acceptable salts include both acid and base addition salts. A “pharmaceutically acceptable salt” can refer to a formulation of a compound that does not cause significant irritation to an organism to which it is administered and/or does not abrogate the biological activity and properties of the compound. In some embodiments, pharmaceutically acceptable salts are obtained by reacting a SMSM compound of the present disclosure with acids. Pharmaceutically acceptable salts are also obtained by reacting a compound of the present disclosure with a base to form a salt.

As used herein, a “small molecular weight compound” can be used interchangeably with “small molecule” or “small organic molecule.” Small molecules refer to compounds other than peptides or oligonucleotides; and typically have molecular weights of less than about 2000 Daltons, e.g., less than about 900 Daltons.

A ribonucleoprotein (RNP) refers to a nucleoprotein that contains RNA. An RNP can be a complex of a ribonucleic acid and an RNA-binding protein. Such a combination can also be referred to as a protein-RNA complex. These complexes can function in a number of biological functions that include, but are not limited to, DNA replication, gene expression, metabolism of RNA, and pre-mRNA splicing. Examples of RNPs include the ribosome, the enzyme telomerase, vault ribonucleoproteins, RNase P, heterogeneous nuclear RNPs (hnRNPs) and small nuclear RNPs (snRNPs).

Nascent RNA transcripts from protein-coding genes and mRNA processing intermediates, collectively referred to as pre-mRNA, are generally bound by proteins in the nuclei of eukaryotic cells. From the time nascent transcripts first emerge from RNA polymerase (e.g., RNA polymerase II) until mature mRNAs are transported into the cytoplasm, the RNA molecules are associated with an abundant set of splicing complex components (e.g., nuclear proteins and snRNAs). These proteins can be components of hnRNPs, which can contain heterogeneous nuclear RNA (hnRNA) (e.g., pre-mRNA and nuclear RNA complexes) of various sizes.

Splicing complex components function in splicing and/or splicing regulation. Splicing complex components can include, but are not limited to, ribonuclear proteins (RNPs), splicing proteins, small nuclear RNAs (snRNAs), small nuclear ribonucleoproteins (snRNPs), and heterogeneous nuclear ribonucleoproteins (hnRNPs). Splicing complex components include, but are not limited to, those that may be required for splicing, such as constitutive splicing, alternative splicing, regulated splicing, and splicing of specific messages or groups of messages. A group of related proteins, the serine/arginine-rich (SR) proteins, can function in constitutive pre-mRNA splicing and may also regulate alternative splice-site selection in a concentration-dependent manner. SR proteins typically have a modular structure that consists of one or two RNA-recognition motifs (RRMs) and a C-terminal rich in arginine and serine residues (RS domain). Their activity in alternative splicing may be antagonized by members of the hnRNP A/B family of proteins. Splicing complex components can also include proteins that are associated with one or more snRNAs. snRNAs in human include, but are not limited to, U1 snRNA, U2 snRNA, U4 snRNA, U5 snRNA, U6 snRNA, U11 snRNA, U12 snRNA, U4atac snRNA, U5 snRNA, and U6atac snRNA. SR proteins in human include, but are not limited to, SC35, SRp55, SRp40, SRm300, SFRS10, TASR-1, TASR-2, SF2/ASF, 9G8, SRp75, SRp30c, SRp20, and P54/SFRS11. Other splicing complex components in human that can be involved in splice site selection include, but are not limited to, U2 snRNA auxiliary factors (e.g. U2AF65, U2AF35), Urp/U2AF1-RS2, SF1/BBP, CBP80, CBP 20, SF1 and PTB/hnRNP1. hnRNP proteins in humans include, but are not limited to, A1, A2/B1, L, M, K, U, F, H, G, R, I and C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In some embodiments, the splicing complex component comprises a nucleic acid, a protein, a carbohydrate, a lipid, a co-factor, a nutrient, a metabolite, or an auxiliary splicing factor. In some embodiments, the splicing complex component comprises an auxiliary splicing factor such as a ribonucleoprotein (RNP) which can be a heterogeneous nuclear ribonucleoprotein (hnRNP) or a small nuclear ribonucleoprotein (snRNP). In some embodiments, the auxiliary splicing factor includes, but are not limited to, 9G8, A1 hnRNP, A2 hnRNP, ASD-1, ASD-2b, ASF, B1 hnRNP, C1 hnRNP, C2 hnRNP, CBP20, CBP80, CELF, F hnRNP, FBP11, Fox-1, Fox-2, G hnRNP, H hnRNP, hnRNP C, hnRNP G, hnRNP K, hnRNP M, hnRNP U, Hu, HUR, K hnRNP, KH-type splicing regulatory protein (KSRP), L hnRNP, M hnRNP, mBBP, muscle-blind like (MBNL), NF45, NFAR, Nova-1, Nova-2, P54/SFRS 11, polypyrimidine tract binding protein (PTBP) 1, PTBP2, PRP19 complex proteins, R hnRNP, RNPC1, SAM68, SC35, SF, SF1/BBP, SF2, SF3 a, SF3B, SFRS10, Sm proteins, SR proteins, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

Splicing complex components may be stably or transiently associated with a snRNP or with a transcript (e.g., pre-mRNA). In some embodiments, the pre-mRNA binds to a splicing complex or a component thereof.

The term “intron” refers to both the DNA sequence within a gene and the corresponding sequence in the unprocessed RNA transcript. As part of the RNA processing pathway, introns can be removed by RNA splicing either shortly after or concurrent with transcription. Introns are found in the genes of most organisms and many viruses. They can be located in a wide range of genes, including those that generate proteins, ribosomal RNA (rRNA), and transfer RNA (tRNA).

An “exon” can be any part of a gene that encodes a part of the final mature RNA produced by that gene after introns have been removed by RNA splicing. The term “exon” refers to both the DNA sequence within a gene and to the corresponding sequence in RNA transcripts.

A “spliceosome” can be assembled from snRNAs and protein complexes. The spliceosome can remove introns from a transcribed pre-mRNA.

The term “cryptic exon” can refer to an intronic sequence that may be flanked by apparent consensus splice sites (e.g., cryptic splice site) but are generally not spliced into the mature mRNA or the product of splicing. The term “poison exon” can refer to a cryptic exon that contains a premature termination codon in the reading frame of the exon when included in an RNA transcript. “Poison exon” can also refer to a cryptic exon inclusion of which in an RNA transcript causes a reading frameshift in downstream exons resulting in a premature stop codon, which was not in frame prior to the frameshift caused by inclusion of the cryptic exon. In some embodiments, the poison exon is a variant of an existing exon. In some embodiments, the poison exon is an extended form of an existing exon. In some embodiments, the poison exon is a truncated form of an existing exon. The terms “poison exon” and “toxic exon” are used interchangeably in the present disclosure. The terms “stop codon” and “termination codon” are used interchangeably in the present disclosure.

A splicing event that promotes inclusion of a poison exon can further promote inclusion of an intron immediately following the poison exon in an RNA transcript. Inclusion of the poison exon and the intron immediately following the poison exon can result in “nuclear retention” of the RNA transcript, e.g., mRNA, wherein the RNA transcript is retained in the nucleus and not transported or exported to the cytoplasm and thus, not translated into a protein.

### Small Molecule Splicing Modulators (SMSMs)

It has now been found that compounds of this disclosure, and pharmaceutically acceptable compositions thereof, are effective as agents for use in treating, preventing, or ameliorating a disease or a condition associated with a target RNA. The present disclosure provides the unexpected discovery that certain small chemical molecules can modify splicing events in pre-mRNA molecules, herein referred to as small molecule splicing modulators (SMSMs). These SMSMs can modulate specific splicing events in specific pre-mRNA molecules. These SMSMs can operate by a variety of mechanisms to modify splicing events. For example, the SMSMs of this disclosure can: 1) interfere with the formation and/or function and/or other properties of splicing complexes, spliceosomes, and/or their components such as hnRNPs, snRNPs, SR-proteins and other splicing factors or elements, resulting in the prevention or induction of a splicing event in a pre-mRNA molecule. As another example, the SMSMs of this disclosure can: 2) prevent and/or modify post-transcriptional regulation (e.g., splicing) of gene products, such as hnRNPs, snRNPs, SR-proteins and other splicing factors, which can subsequently be involved in the formation and/or function of a spliceosome or splicing complex component; 3) prevent and/or modify phosphorylation, glycosylation and/or other modifications of gene products including, but not limited to, hnRNPs, snRNPs, SR-proteins and other splicing factors, which can subsequently be involved in the formation and/or function of a spliceosome or splicing complex component; or 4) bind to and/or otherwise affect specific pre-mRNA so that a specific splicing event is prevented or induced, e.g., via a mechanism that does not involve base-pairing with RNA in a sequence-specific manner. The small molecules of this disclosure are different from and are not related to antisense or antigene oligonucleotides.

Described herein are compounds modifying splicing of gene products for use in the treatment, prevention, and/or delay of progression of diseases or conditions. Described herein are compounds modifying splicing of gene products wherein the compounds induce a transcriptionally inactive variant or transcript of a gene product. Described herein are compounds modifying splicing of gene products wherein the compounds induce a transcriptionally active variant or transcript of a gene product. Described herein are compounds modifying splicing of gene products wherein the compounds repress a transcriptionally active variant or transcript of a gene product. Described herein are compounds modifying splicing of gene products wherein the compounds repress a transcriptionally inactive variant or transcript of a gene product.

Described herein are compounds modifying splicing of gene products wherein the compounds induce a specific variant or isoform of a mature mRNA. Described herein are compounds modifying splicing of gene products wherein the compounds cause increased expression of a protein encoded by a variant or an isoform of an mRNA derived from a pre-mRNA that the compounds bind. Described herein are compounds modifying splicing of gene products wherein the compounds cause increased expression of a variant or an isoform of an mRNA derived from a pre-mRNA that the compounds bind, leading to increased expression of the protein encoded by the variant or the isoform of the mRNA. Described herein are compounds modifying splicing of gene products wherein the compounds cause increased expression of a variant or an isoform of an mRNA containing a specific exon by inducing exon inclusion in a pre-mRNA that compounds bind, leading to increased expression of the protein encoded by the variant or the isoform of the mRNA. Described herein are compounds modifying splicing of gene products wherein the compounds cause exon inclusion in a pre-mRNA that compounds bind, leading to increased expression of the protein encoded by the specific variant or the isoform of the mRNA.

Described herein are compounds modifying splicing of gene products wherein the compounds repress a specific variant or isoform of a mature mRNA. Described herein are compounds modifying splicing of gene products wherein the compounds cause decreased expression of a protein encoded by a variant or an isoform of an mRNA derived from a pre-mRNA that the compounds bind. Described herein are compounds modifying splicing of gene products wherein the compounds cause decreased expression of a variant or an isoform of an mRNA derived from a pre-mRNA that the compounds bind, leading to decreased expression of the protein encoded by the variant or the isoform of the mRNA. Described herein are compounds modifying splicing of gene products wherein the compounds cause decreased expression of a variant or an isoform of an mRNA containing a specific exon by inducing exon inclusion in a pre-mRNA that compounds bind, leading to decreased expression of the protein encoded by the variant or the isoform of the mRNA. Described herein are compounds modifying splicing of gene products wherein the compounds cause exon inclusion in a pre-mRNA that compounds bind, leading to decreased expression of the protein encoded by the specific variant or the isoform of the mRNA.

Described herein are compounds modifying splicing of gene products wherein the compounds induce a post-transcriptionally inactive variant or transcript of a gene product. Described herein are compounds modifying splicing of gene products wherein the compounds repress a post-transcriptionally active variant or transcript of a gene product. Described herein are compounds modifying splicing of gene products wherein the compounds induce a post-transcriptionally destabilized variant or transcript of a gene product. Described herein are compounds modifying splicing of gene products wherein the compounds cause less expression of a protein encoded by an mRNA derived from a pre-mRNA that the compounds bind. Described herein are compounds modifying splicing of gene products wherein the compounds cause less expression of an mRNA derived from a pre-mRNA that the compounds bind, leading to decreased expression of the protein encoded by the mRNA. Described herein are compounds modifying splicing of gene products wherein the compounds cause increased expression of an mRNA containing a poison exon derived from a pre-mRNA that compounds bind, leading to decreased expression of the protein encoded by the mRNA. Described herein are compounds modifying splicing of gene products wherein the compounds cause nonsense-mediated decay (NMD) of an mRNA derived from a pre-mRNA that compounds bind, leading to decreased expression of the protein encoded by the mRNA.

In one aspect, a SMSM described herein is a compound of Formula (I), or a pharmaceutically acceptable salt thereof:

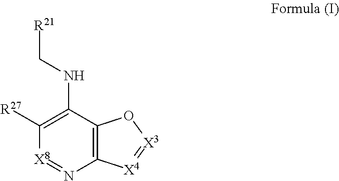

wherein:
X³ is selected from the group consisting of N, and CR²³;
X⁴ is selected from the group consisting of N, and CR²⁴;
X⁸ is CR²⁸ or N;
R²¹ is selected from the group consisting of phenyl, 5-6 membered heteroaryl, and 5-6 membered heterocycloalkyl, each of which is unsubstituted or substituted with 1, 2, 3 or 4, independently selected R^{1A} groups; each R^{1A} is independently selected from halo, CN, NO₂, C₁₋₆ alkyl, C₂₋₆ alkenyl, C₂₋₆ alkynyl, C₁₋₆ haloalkyl, C₁₋₄ haloalkoxy, C₁₋₆ alkoxy, —C(═O)OH, —C(═O)C₁₋₆ alkyl, —C(═O)C₁₋₆ haloalkyl, and —C(═O)C₁₋₆ alkoxy;
R²³ is selected from the group consisting of H, azido, halo, —CN, —NO₂, C₁₋₆ alkyl, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, 4-10 membered heterocycloalkyl, —(C₁₋₆ alkylene)-C₃₋₁₀ cycloalkyl, —(C₁₋₆ alkylene)-4-10 membered heterocycloalkyl, —(C₁₋₆ heteroalkylene)-C₃₋₁₀ cycloalkyl, —(C₁₋₆ heteroalkylene)-4-10 membered heterocycloalkyl, —(C₁₋₆ alkylene)-C₆₋₁₀ aryl, —(C₁₋₆ alkylene)-5-10 membered heteroaryl, —(C₁₋₆ heteroalkylene)-C₆₋₁₀ aryl, —(C₁₋₆ heteroalkylene)-5-10 membered heteroaryl, —OR^{a3}, —SR^{a3}, —C(═O)R^{b3}, —C(═O)OR^{b3}, —NR^{c3}R^{d3}, —C(═O)NR^{c3}R^{d3}, —OC(═O)NR^{c3}R^{d3}, —NR^{c3}C(═O)R^{b3}, —NR^{c3}C(═O)OR^{b3}, —NR^{c3}C(═O)NR^{c3}R^{d3}, —NR^{c3}S(═O)₂R^{b3}, —NR^{c3}S(═O)₂NR^{c3}R^{d3}, —S(O)NR^{c3}R^{d3}, and —S(O)₂NR^{c3}R^{d3}, wherein the C₁₋₆ alkyl, C₁₋₆ alkylene, C₁₋₆ heteroalkylene, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, and 4-10 membered heterocycloalkyl are each unsubstituted or substituted with 1, 2, 3, 4, 5, 6, 7, or 8 independently selected R²⁰ groups;
R²⁴ is selected from the group consisting of —C≡CH, H, azido, halo, —CN, —NO₂, C₁₋₆ alkyl, C₂₋₆ alkenyl, C₁₋₆ heteroalkyl, C₃₋₁₀ cycloalkyl, C₆₋₁₀ aryl, 5-10 membered heteroaryl, 4-10 membered heterocycloalkyl, —OR^{a4}, —C(═O)R^{b4}, —C(═O)OR^{b4}, —NR^{c4}R^{d4}, —C(═O)NR^{c4}R^{d4}, —OC(═O)NR^{c4}R^{d4}, —NR^{c4}C(═O)R^{b4}, —NR^{c4}C(═O)OR^{b4}, —NR^{c4}C(═O)NR^{c4}R^{d4}, —NR^{c4}S(═O)₂R⁴, —NR^{c4}S(═O)₂NR^{c4}R^{d4}, —S(O)NR^{c4}R^{d4}, and —S(O)₂NR^{c4}R^{d4}, wherein the C₁₋₆ alkyl, C₂₋₆ alkenyl, C₁₋₆ heteroalkyl, C₃₋₁₀ cycloalkyl, C₆₋₁₀ aryl, 5-10 membered heteroaryl, and 4-10 membered heterocycloalkyl, are each unsubstituted or substituted with 1, 2, 3, or 4 independently selected R^{20d} groups;
each R^{20d} is independently selected from the group consisting of —OH, —SH, —CN, —NO₂, halogen, oxo, amino, carbamyl, carbamoyl, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino;
R²⁷ is selected from the group consisting of H, azido, halo, C₁₋₆ alkyl, C₂₋₆ alkenyl, C₂₋₆ alkynyl, C₁₋₆ heteroalkyl, —CN, —NO₂, —OR^{a7}, —C(═O)R^{b7}, —C(═O)OR^{b7}, —NR^{c7}R^{d7}, —C(═O)NR^{c7}R^{d7}, —OC(═O)NR^{c7}R^{d7}, —NR^{c7}C(═O)R^{b7}, —NR^{c7}C(═O)OR^{b7}, —NR^{c7}C(═O)NR^{c7}R^{d7}, —NR^{c7}S(═O)₂R^{b7}, and —NR^{c7}S(═O)₂NR^{c7}R^{d7} wherein the C₁₋₆ alkyl, C₁₋₆ heteroalkyl, C₂₋₆ alkenyl, and C₂₋₆ alkynyl are each unsubstituted or substituted with 1, 2, 3, or 4 independently selected R²⁰ groups;
R²⁸ is selected from the group consisting of H, oxo, azido, halo, C₁₋₆ alkyl, C₂₋₆ alkenyl, C₂₋₆ alkynyl, C₁₋₆ heteroalkyl, —CN, —NO₂, —OR^{a8}, —C(═O)R^{b8}, —C(═O)OR^{b8}, —NR^{c8}R^{d8}, —C(═O)NR^{c8}R^{d8}, —OC(═O)NR^{c8}R^{d8}, —NR^{c8}C(═O)R^{d8}, —NR^{c8}C(═O)OR^{b8}, —NR^{c8}C(═O)NR^{c8}R^{dc}, —NR^{c8}S(═O)₂R^{d8}, and —NR^{c8}S(═O)₂NR^{c8}R^{d8}, wherein the C₁₋₆ alkyl, C₁₋₆ heteroalkyl, C₂₋₆ alkenyl, and C₂₋₆ alkynyl are each unsubstituted or substituted with 1, 2, 3, or 4 independently selected R²⁰ groups;
each R^{a3}, R^{b3}, R^{c3}, R^{d3}, R^{a4}, R^{b4}, R^{c4}, R^{d4}, R^{a7}, R^{b7}, R^{c7}, R^{d7} R^{a8}, R^{b8}, R^{c8}, and R^{d8} is independently selected from the group consisting of H, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, and 4-10 membered heterocycloalkyl each of which is unsubstituted or substituted with 1, 2, 3, or 4 independently selected R²⁰ groups;
   or R^{c3} and R^{d3} together with the N atom to which they are connected, come together to form a 5-10 membered heteroaryl or a 4-10 membered heterocycloalkyl ring, each of which is unsubstituted or substituted with 1, 2, 3, or 4 independently selected R²⁰ groups;
   or R^{c4} and R^{d4} together with the N atom to which they are connected, come together to form a 5-10 membered heteroaryl or a 4-10 membered heterocycloalkyl ring, each of which is unsubstituted or substituted with 1, 2, 3, or 4 independently selected R²⁰ groups; and
each R²⁰ is independently selected from the group consisting of —OH, —SH, —CN, —NO₂, halo, oxo, amino, carbamyl, carbamoyl, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments of a compound of Formula (I) or (Ia), R²³ is substituted with 1, 2, 3, or 4 independently selected R²⁰ groups.

In some embodiments, the compound of the disclosure is not

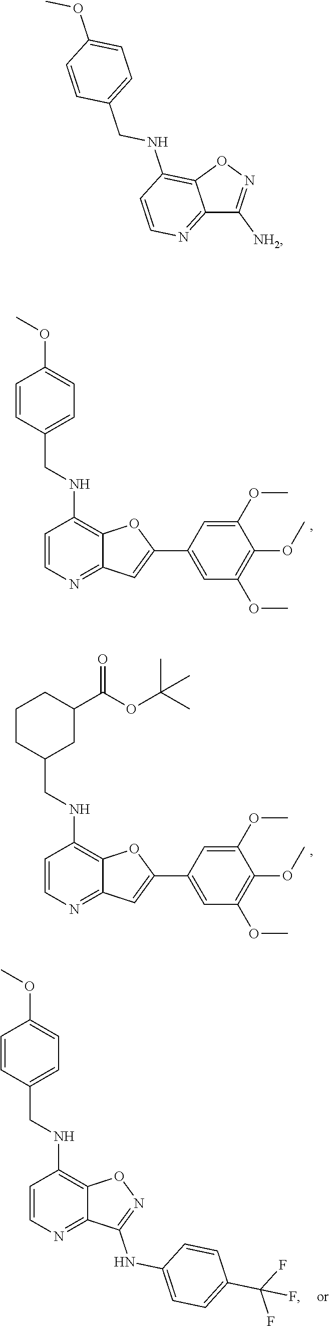 [SMILES: COc1ccc(CNc2ccnc3c(N)noc23)cc1 ; COc1ccc(CNc2ccnc3cc(-c4cc(OC)c(OC)c(OC)c4)oc23)cc1 ; COc1cc(-c2cc3nccc(NCC4CCCC(C(=O)OC(C)(C)C)C4)c3o2)cc(OC)c1OC ; COc1ccc(CNc2ccnc3c(Nc4ccc(C(F)(F)F)cc4)noc23)cc1]

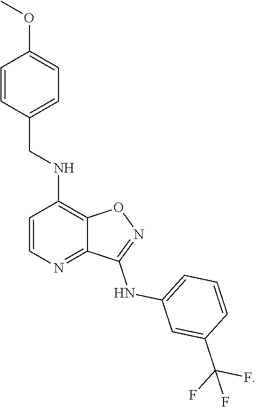 [SMILES: COc1ccc(CNc2ccnc3c(Nc4cccc(C(F)(F)F)c4)noc23)cc1]

In some embodiments, X⁴ is CR²⁴. In some embodiments, R²⁴ is hydrogen. In some embodiments, R²⁴ is halogen. In some embodiments, R²⁴ is —Br. In some embodiments, R²⁴ is —F. In some embodiments of a compound of Formula (I) or (Ia), R²⁴ is —Cl. In some embodiments, R²⁴ is —CN. In some embodiments, R²⁴ is C₁₋₄ alkyl. In some embodiments, R²⁴ is methyl. In some embodiments, R²⁴ is ethyl. In some embodiments, R²⁴ is cycloalkyl. In some embodiments, R²⁴ is cyclopropyl. In some embodiments, R²⁴ is —C≡CH.

In some embodiments of a compound of Formula (I) or (Ia), X⁸ is CR²⁸. In some embodiments, X⁸ is CR²⁸, wherein R²⁸ is hydrogen. In some embodiments, X⁸ is N.

In some embodiments, disclosed herein is a compound of the Formula (Ia):

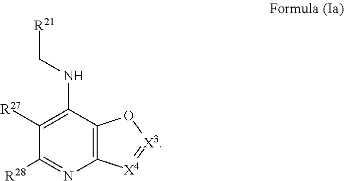

In some embodiments, the compound of Formula (Ia) has a structure of

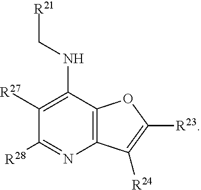

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is selected from the group consisting of phenyl and 5-6 membered heteroaryl, each of which is unsubstituted or substituted with 1, 2, 3 or 4, independently selected R^{1A} groups; each R^{1A} is independently selected from halo, CN, NO₂, C₁₋₆ alkyl, C₂₋₆ alkenyl, C₂₋₆ alkynyl, C₁₋₆ haloalkyl, C₁₋₆ alkoxy, —C(═O)OH, —C(═O)C₁₋₆ alkyl, —C(═O)C₁₋₆ haloalkyl, and —C(═O)C₁₋₆ alkoxy. In some embodiments, R²¹ is unsubstituted or substituted 5 membered heteroaryl. In some embodiments, R²¹ is unsubstituted or substituted 5 membered heterocycloalkyl. In some embodiments, R²¹ is unsubstituted. In some embodiments, R²¹ is substituted with 1, 2, or 3, independently selected R^{1A} groups; wherein each R^{1A} is independently selected from halo, CN, NO₂, alkyl, alkenyl, C₂₋₆ alkynyl, alkoxy, —C(═O)OH, an ether group, or an ester group, each of which is unsubstituted or substituted. In some embodiments, R²¹ is substituted with 1, 2, or 3 substituents independently selected R^{1A} groups; wherein each R^{1A} is independently selected from halo, C₁₋₆alkyl, C₁₋₆haloalkyl, and C₁₋₆alkoxy. In some embodiments, R²¹ is substituted with 1, 2, or 3 substituents independently selected R^{1A} groups; wherein each R^{1A} is independently selected from halo, C₁₋₃alkyl, C₁₋₃haloalkyl, and C₁₋₃alkoxy. In some embodiments, R²¹ is

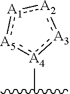

wherein [symbol] represents a single or a double bond; each of A₁, A₂, A₃, and A₅ is independently selected from the group consisting of O, S, N, NH, NR^{1A}, CH, CR^{1A}, CH₂, and CHR^{1A}; and A₄ is selected from the group consisting of N, C, CH and CR^{1A}. In some embodiments, R²¹ is

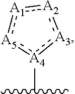

wherein [symbol] represents a single or a double bond; each of A₁, A₂, A₃, and A₅ is independently selected from the group consisting of O, S, N, NH, NR^{1A}, C, CH, CR^{1A}, CH₂, and CHR^{1A}; and A4 is selected from the group consisting of N, C, CH and CR^{1A}.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is selected from the group consisting of

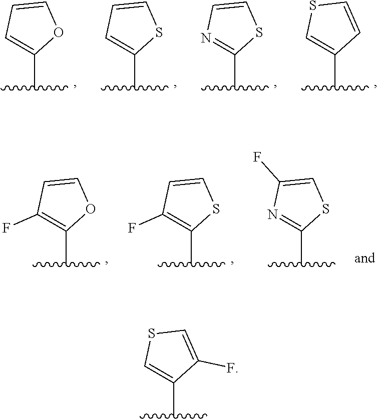

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is 5 membered heteroaryl. In some embodiments, R²¹ is furanyl, or thiazolyl each of which is substituted or unsubstituted.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is unsubstituted furanyl. In some embodiments, R²¹ is substituted furanyl. In some embodiments, R²¹ is unsubstituted thiazolyl.

In some embodiments, R²¹ is substituted thiazolyl.

In some embodiments, R²¹ is

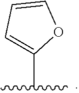 [SMILES: *c1ccco1]

In some embodiments, R²¹ is

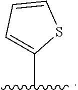 [SMILES: *c1cccs1]

In some embodiments, R²¹ is

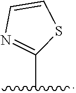 [SMILES: *c1nccs1]

In some embodiments, R²¹ is

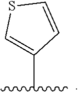 [SMILES: *c1ccsc1]

In some embodiments, R²¹ is

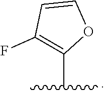 [SMILES: *c1occc1F]

In some embodiments, R²¹ is

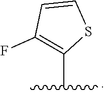 [SMILES: *c1sccc1F]

In some embodiments, R²¹ is

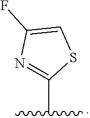 [SMILES: *c1nc(F)cs1]

In some embodiments, R²¹ is

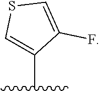 [SMILES: *c1cscc1F]

In some embodiments, R²¹ is

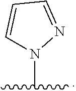 [SMILES: *n1cccn1]

In some embodiments, R²¹ is

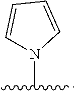 [SMILES: *n1cccc1]

In some embodiments, R²¹ is

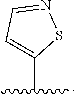 [SMILES: *c1ccns1]

In some embodiments, R²¹ is

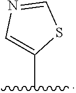 [SMILES: *c1cncs1]

In some embodiments, R²¹ is

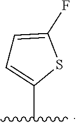 [SMILES: *c1ccc(F)s1]

In some embodiments, R²¹ is

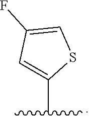 [SMILES: *c1cc(F)cs1]

In some embodiments, R²¹ is

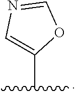 [SMILES: *c1cnco1]

In some embodiments, R²¹ is

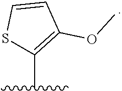 [SMILES: *c1sccc1OC]

In some embodiments, R²¹ is

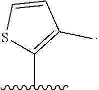 [SMILES: *c1sccc1C]

In some embodiments, R²¹ is

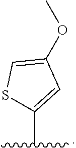 [SMILES: *c1cc(OC)cs1]

In some embodiments, R²¹ is

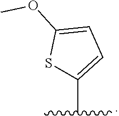 [SMILES: *c1ccc(OC)s1]

In some embodiments, R²¹ is

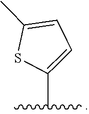 [SMILES: *c1ccc(C)s1]

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is

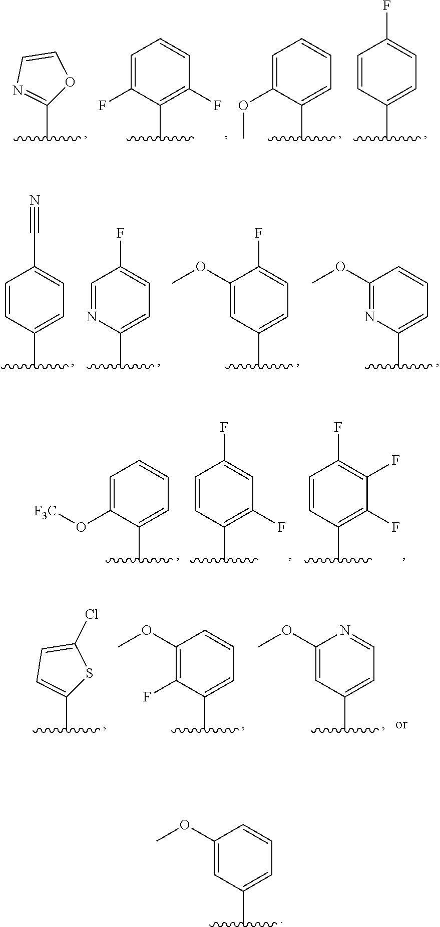

In some embodiments, R²¹ is

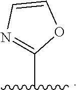 [SMILES: *c1ncco1]

In some embodiments, R²¹ is

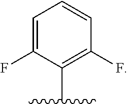 [SMILES: *c1c(F)cccc1F]

In some embodiments, R²¹ is

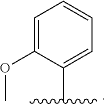 [SMILES: *c1ccccc1OC]

In some embodiments, R²¹ is

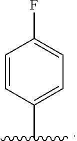 [SMILES: *c1ccc(F)cc1]

In some embodiments, R²¹ is

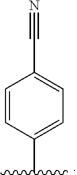 [SMILES: *c1ccc(C#N)cc1]

In some embodiments, R²¹ is

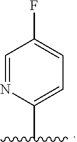 [SMILES: *c1ccc(F)cn1]

In some embodiments, R²¹ is

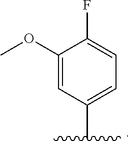 [SMILES: *c1ccc(F)c(OC)c1]

In some embodiments, R²¹ is

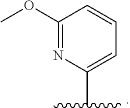 [SMILES: *c1cccc(OC)n1]

In some embodiments, R²¹ is

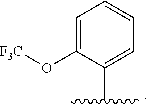 [SMILES: *c1ccccc1OC(F)(F)F]

In some embodiments, R²¹ is

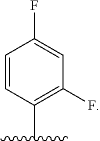 [SMILES: *c1ccc(F)cc1F]

In some embodiments, R²¹ is

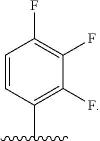 [SMILES: *c1ccc(F)c(F)c1F]

In some embodiments, R²¹ is

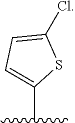 [SMILES: *c1ccc(Cl)s1]

In some embodiments, R²¹ is

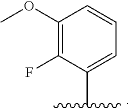 [SMILES: *c1cccc(OC)c1F]

In some embodiments, R²¹ is

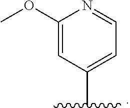 [SMILES: *c1ccnc(OC)c1]

In some embodiments, R²¹ is

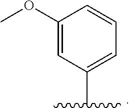 [SMILES: *c1cccc(OC)c1]

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is unsubstituted or substituted phenyl. In some embodiments, R²¹ is unsubstituted or substituted 6 membered heteroaryl. In some embodiments, R²¹ is unsubstituted or substituted 6 membered heterocycloalkyl. In some embodiments, R²¹ is unsubstituted. In some embodiments, R²¹ is substituted with 1, 2, 3, or 4 independently selected R^{1A} groups; wherein each R R^{A} is independently selected from halo, CN, NO₂, alkyl, alkenyl, C₂₋₆ alkynyl, alkoxy, —C(O)OH, an ether group, or an ester group, each of which is unsubstituted or substituted. In some embodiments, R²¹ is substituted with 1, 2, 3, or 4 substituents independently selected R^{1A} groups; wherein each R^{1A} is independently selected from halo, C₁₋₆alkyl, C₁₋₆haloalkyl, and C₁₋₆alkoxy. In some embodiments, R²¹ is substituted with 1, 2, 3, or 4 substituents independently selected R^{1A} groups; wherein each R^{1A} is independently selected from halo, C₁₋₃alkyl, C₁₋₃haloalkyl, and C₁₋₃alkoxy. In some embodiments, R²¹ is

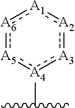

wherein [symbol] represents a single or a double bond; each of A₁, A₂, A₃, A₅ and A₆ is independently selected from the group consisting of O, S, N, NH, NR^{1A} CH, CR^{1A}, CH₂, and CHR^{1A}; and A₄ is selected from the group consisting of N, C, CH and CR^{1A}. In some embodiments, R²¹ is

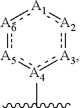

wherein [symbol]
represents a single or a double bond; each of A₁, A₂, A₃, A₅ and A₆ is independently selected from the group consisting of O, S, N, NH, NR^{1A}, C, CH, CR^{1A}, CH₂, and CHR^{1A}; and A₄ is selected from the group consisting of N, C, CH, and CR^{1A}. In some embodiments, R²¹ is selected from the group consisting of

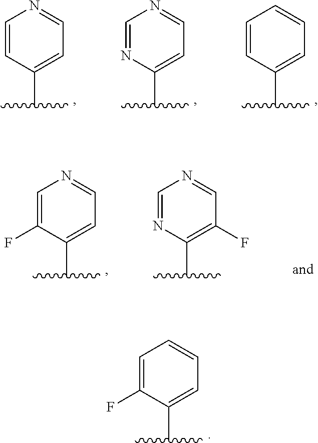 [SMILES: *c1ccncc1 ; *c1ccncn1 ; *c1ccccc1 ; *c1ccncc1F ; *c1ncncc1F ; *c1ccccc1F]

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is substituted or unsubstituted phenyl. In some embodiments, R²¹ is 6 membered heteroaryl. In some embodiments, R²¹ is pyridinyl, thiophenyl, pyrimidinyl, each of which is substituted or unsubstituted.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is unsubstituted pyridinyl. In some embodiments, R²¹ is substituted pyridinyl. In some embodiments, R²¹ is unsubstituted thiophenyl. In some embodiments, R²¹ is substituted thiophenyl. In some embodiments, R²¹ is unsubstituted pyrimidinyl. In some embodiments, R²¹ is substituted pyrimidinyl.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²¹ is

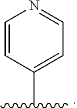 [SMILES: *c1ccncc1]

In some embodiments R²¹ is

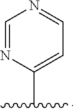 [SMILES: *c1ccncn1]

In some embodiments, R²¹ is

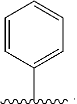 [SMILES: *c1ccccc1]

In some embodiments, R²¹ is

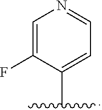 [SMILES: *c1ccncc1F]

In some embodiments, R²¹ is

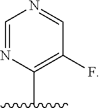 [SMILES: *c1ncncc1F]

In some embodiments, R²¹ is

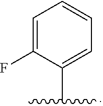 [SMILES: *c1ccccc1F]

In some embodiments of a compound of Formula (I) or (Ia), X³ is CH. In some embodiments, X³ is CR²³. In some embodiments, X³ is CR²³, wherein R²³ is C₁₋₆ alkyl or C₁₋₆ heteroalkyl, wherein the C₁₋₆ alkyl and C₁₋₆ heteroalkyl are unsubstituted or substituted independently with 1, 2, 3, or 4 R²⁰ groups. In some embodiments, X³ is CCH₂CHNH₂CH₃. In some embodiments, X³ is CCH₂CHNH₂CH₂OH. In some embodiments, X³ is CCH₂CHNH₂CH₂CH₃. In some embodiments, X³ is CCH₂CHNH₂CH₂CH₂OH. In some embodiments, X³ is CCH₂CHNH₂CH₂CH₂F. In some embodiments, X³ is CCH₂CHNH₂CH₂CHF₂. In some embodiments, X³ is CCH₂CHNH₂CH₂CH(CH₃)₂. In some embodiments, X⁴ is N. In some embodiments, X⁴ is CH. In some embodiments, X⁴ is CR²⁴, wherein R²⁴ is selected from the group consisting of halo, CN, and substituted or unsubstituted C₁₋₆ alkyl. In some embodiments, X⁴ is CCl. In some embodiments, X⁴ is CBr. In some embodiments, X⁴ is CF. In some embodiments, X⁴ is CCN. In some embodiments, X⁴ is CCH₃. In some embodiments, X⁴ is C(cyclopropyl). In some embodiments, X⁸ is N. In some embodiments, X⁸ is CR²⁸.

In some embodiments of a compound of Formula (I), (Ia), (IIa), (IIb), (IIc), (IId), (IIIa), (IIb), (IIc), (IIId), (IIIe), (IIIf), (IIIg), or (IIIh), R²³ (or,

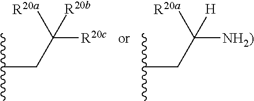

is selected from the group consisting of C₁₋₆ alkyl, C₂₋₆ alkenyl, C₂₋₆ alkynyl, C₁₋₆ heteroalkyl, —(C₁₋₆ alkylene)-C₃₋₁₀ cycloalkyl, —(C₁₋₆ alkylene)-4-10 membered heterocycloalkyl, —(C₁₋₆ heteroalkylene)-C₃₋₁₀ cycloalkyl, —(C₁₋₆ heteroalkylene)-4-10 membered heterocycloalkyl, —(C₁₋₆ alkylene)-C₆₋₁₀ aryl, —(C₁₋₆ alkylene)-5-10 membered heteroaryl, —(C₁₋₆ heteroalkylene)-C₆₋₁₀ aryl, and —(C₁₋₆ heteroalkylene)-5-10 membered heteroaryl, wherein the C₁₋₆ alkyl, C₁₋₆ alkylene, C₁₋₆ heteroalkylene, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, and 4-10 membered heterocycloalkyl are each unsubstituted or substituted with 1, 2, 3, 4, 5, 6, 7, or 8 independently selected R²⁰ groups. In some embodiments, R²³ is substituted or unsubstituted C₁₋₆ alkyl or substituted or unsubstituted C₁₋₆ heteroalkyl. In some embodiments, R²³ is substituted or unsubstituted C₁₋₆ heteroalkyl. In some embodiments, the C₁₋₆ heteroalkyl is —CH₂CH(NH₂)CH₂—S(═O)₂—CH₃ or —CH₂CH(NH₂)CH₂—S(═O)—CH₃. In some embodiments, R²³ is —CH₂CH(NH₂)CH₂—S(═O)₂—CH₃. In some embodiments, R²³ is —CH₂CH(NH₂)CH₂—S(═O)—CH₃. In some embodiments, R²³ is CH₂CHNH₂CH₃. In some embodiments, R²³ is CH₂CHNH₂CH₂OH. In some embodiments, R²³ is CH₂CHNH₂CH₂CH₃. In some embodiments, R²³ is CH₂CHNH₂CH₂CH₂OH. In some embodiments, R²³ is CH₂CHNH₂CH₂CH₂F. In some embodiments, R²³ is CH₂CHNH₂CH₂CHF₂. In some embodiments, R²³ is CH₂CHNH₂CH₂CH(CH₃)₂. In some embodiments, R²³ is CH₂CHNH₂CHFCH₃. In some embodiments, R²³ is CH₂CHNH₂CH₂F. In some embodiments, R²³ is CH₂CHNH₂CH₂OCH₃. In some embodiments, R²³ is CH₂CHNH₂CH₂OCD₃. In some embodiments, R²³ is CF₂CH(NH₂)CH₃. In some embodiments, R²³ is CH₂CH(NH₂)CH₂OCH₃. In some embodiments, R²³ is CF₂CH(NH₂)CH₃. In some embodiments, R²³ is CH₂CH(NH₂)CHF₂.

In some embodiments of a compound of Formula (I) or (Ia), R²³ is H.

In some embodiments of a compound of Formula (I) or (Ia), R²³ is substituted with 1, 2, or 3 independently selected R²⁰ groups, wherein each R²⁰ group is independently selected from the group consisting of OH, SH, CN, NO₂, halo, oxo, amino, C₁₋₃alkyl, C₁₋₃alkoxy, cycloalkyl, aryl, heteroaryl, heterocycloalkyl, carbamyl, and carbamoyl. In some embodiments, R²³ is substituted with 1, 2, or 3 independently selected R²⁰ groups, wherein each R²⁰ group is independently selected from the group consisting of OH, halo, and C₁₋₃alkoxy.

In some embodiments of a compound of Formula (I) or (Ia), R²³ is substituted or unsubstituted C₁₋₆ alkyl. In some embodiments, R²³ is C₁₋₆ alkyl, wherein C₁₋₆ alkyl is substituted with 1, 2, or 3 independently selected R²⁰ groups. In some embodiments, R²³ is C₁₋₆ alkyl, wherein C₁₋₆ alkyl is substituted with 1, 2, or 3 independently selected R²⁰ groups, wherein each R²⁰ group is independently selected from the group consisting of OH, SH, CN, NO₂, halo, oxo, amino, C₁₋₃alkyl, C₁₋₃alkoxy, cycloalkyl, aryl, heteroaryl, heterocycloalkyl, carbamyl, and carbamoyl. In some embodiments, R²³ is C₁₋₆ alkyl, wherein C₁₋₆ alkyl is substituted with 1, 2, or 3 independently selected R²⁰ groups, wherein each R²⁰ group is independently selected from the group consisting of OH, halo, and C₁₋₃alkoxy.

In some embodiments of a compound of Formula (I) or (Ia), R²³ is substituted or unsubstituted C₁₋₆ alkenyl. In some embodiments, R²³ is C₁₋₆ alkenyl, wherein C₁₋₆ alkenyl is substituted with 1, 2, or 3 independently selected R²⁰ groups.

In some embodiments of a compound of Formula (I) or (Ia), R²³ is substituted or unsubstituted C₂₋₆ alkynyl. In some embodiments, R²³ is C₂₋₆ alkynyl, wherein C₂₋₆ alkynyl is substituted with 1, 2, or 3 independently selected R²⁰ groups.

In some embodiments of a compound of Formula (I), (Ia), (Ila), (IIb), (IIc), (IId), (IIIa), (IIIb), (IIIc), (IIId), (IIIe), (IIIf), (IIIg), or (IIIh), each R²⁰ is independently selected from the group consisting of —OH, —SH, —CN, —NO₂, halogen, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 4-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, and wherein each of the alkyl, alkenyl, phenyl, cycloalkyl, alkenyl, heteroaryl, and heterocycloalkyl is optionally substituted with 1, 2, 3, or 4 groups independently selected from OH, —SH, —CN, —NO₂, halogen, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, C₁₋₄ haloalkoxy, C₃₋₆ cycloalkyl, amino, oxo, C₁₋₄ alkylamino, and di(C₁₋₄ alkyl)amino.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), each R²⁰ is independently selected from the group consisting of —OH, —SH, —CN, —NO₂, halogen, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, C₁₋₄ haloalkoxy, C₃₋₆ cycloalkyl, phenyl, 5-6 membered heteroaryl, 4-6 membered heterocycloalkyl, amino, C₁₋₄ alkylamino, and di(C₁₋₄ alkyl)amino, and wherein each of the alkyl, alkenyl, phenyl, cycloalkyl, alkenyl, heteroaryl, and heterocycloalkyl is optionally substituted with 1, 2, 3, or 4 groups independently selected from OH, —SH, —CN, —NO₂, halogen, oxo, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, C₁₋₄ haloalkoxy, C₃₋₆ cycloalkyl, amino, C₁₋₄ alkylamino, and di(C₁₋₄ alkyl)amino.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), each R²⁰ is independently selected from the group consisting of —OH, —SH, —CN, —NO₂, halogen, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ heteroalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 4-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein alkyl, alkenyl, alkynyl, heteroalkyl, cycloalkyl, phenyl, heteroaryl and heterocycloalkyl are each optionally substituted with 1, 2, 3 or 4 R³¹ groups, wherein each R³¹ is independently —OH, —SH, —CN, —NO₂, halogen, oxo, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy. In some embodiments, R³¹ is —OH. In some embodiments, R³¹ is —SH. In some embodiments, R³¹ is —CN. In some embodiments, R³¹ is —NO₂. In some embodiments, R³¹ is halogen. In some embodiments, R³¹ is oxo. In some embodiments, R³¹ is C₁₋₄ alkyl. In some embodiments, R³¹ is C₂₋₄ alkenyl. In some embodiments, R³¹ is C₁₋₄ haloalkyl. In some embodiments, R³¹ is C₁₋₄ cyanoalkyl. In some embodiments, R³¹ is C₁₋₄ hydroxyalkyl. In some embodiments, R³¹ is C₁₋₄ alkoxy.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), each R²⁰ is independently selected from the group consisting of —OH, —SH, —CN, —NO₂, halogen, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ heteroalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 4-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein alkyl, alkenyl, alkynyl, heteroalkyl, cycloalkyl, phenyl, heteroaryl and heterocycloalkyl are each optionally substituted with 1, 2, 3 or 4 R³¹ groups, wherein each R³¹ is independently oxo, halogen, methyl, ethyl, —CN, —CF₃, —OH, —OMe, —NH₂, or —NO₂. In some embodiments, R³¹ is oxo. In some embodiments, R³¹ halogen. In some embodiments, R³¹ methyl, ethyl. In some embodiments, R³¹, —CN. In some embodiments, R³¹, —CF₃. In some embodiments, R³¹, —OH. In some embodiments, R³¹, —OMe. In some embodiments, R³¹, —NH₂. In some embodiments, R³¹, —NO₂.

In some embodiments of a compound of Formula (I) or (Ia), R²³ is C₁₋₆ alkyl or C₁₋₆ heteroalkyl, and wherein the C₁₋₆ alkyl and C₁₋₆ heteroalkyl are substituted independently with 1, 2, or 3 R²⁰ groups. In some embodiments, R²³ is C₁₋₆ heteroalkyl, wherein the C₁₋₆ heteroalkyl is substituted with 1, 2, or 3 independently selected R²⁰ groups. In some embodiments, C₁₋₆ alkyl is substituted with 1 R²⁰ group. In some embodiments, C₁₋₆ alkyl is substituted with 2 independently selected R²⁰ groups. In some embodiments, C₁₋₆ alkyl is substituted with 3 independently selected R²⁰ groups. In some embodiments, C₁₋₆ heteroalkyl is substituted with 1 R²⁰ group. In some embodiments, C₁₋₆ heteroalkyl is substituted with 2 independently selected R²⁰ groups. In some embodiments, C₁₋₆ heteroalkyl is substituted with 3 independently selected R²⁰ groups.

In some embodiments of a compound of Formula (I) or (Ia), R²³ is selected from the group consisting of hydrogen, oxo, azido, halogen, —CN, —NO₂, C₁₋₆ haloalkyl, C₁₋₆ alkyl, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, 4-10 membered heterocycloalkyl, —OR^{a3}, —SR^{a3}, —C(═O)R^{b3}, —C(═O)OR^{b3}, —NR^{c3}R^{d3}, —C(═O)NR^{c3}R^{d3}, —OC(═O)NR^{c3}R^{d3}, —NR^{c3}C(═O)R^{b3}, —NR^{c3}C(═O)OR^{b3}, —NR^{c3}C(═O)NR^{c3}R^{d3}, —NR^{c3}S(═O)₂R^{b3}, —NR^{c3}S(═O)₂NR^{c3}R^{d3}, —S(O)NR^{c3}R^{d3}, and —S(O)₂NR^{c3}R^{d3}, wherein the C₁₋₆ alkyl, C₁₋₆ haloalkyl, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, and 4-10 membered heterocycloalkyl are unsubstituted or substituted independently with 1, 2, 3, or 4 R²⁰ groups.

In some embodiments, R²³ is selected from the group consisting of hydrogen, oxo, azido, halogen, —CN, —NO₂, C₁₋₆ haloalkyl, C₁₋₆ alkyl, C₁₋₆ heteroalkyl, C₃₋₁₀ cycloalkyl, C₆₋₁₀ aryl, 5-10 membered heteroaryl, 4-10 membered heterocycloalkyl, —OR^{a3}, and —NR^{c3}R^{d3}, wherein the C₁₋₆ alkyl, C₁₋₆ haloalkyl, C₁₋₆ heteroalkyl, C₃₋₁₀ cycloalkyl, C₆₋₁₀ aryl, 5-10 membered heteroaryl, and 4-10 membered heterocycloalkyl are unsubstituted or substituted. In some embodiments, R²³ is hydrogen.

In some embodiments of a compound of Formula (I) or (Ia), R²³ is —NR^{c3}R^{d3}. In some embodiments, R^{c3} and R^{d3} together with the N atom to which they are connected, come together to form a 5-10 membered heteroaryl or 4-10 membered heterocycloalkyl, wherein the 5-10 membered heteroaryl and 4-10 membered heterocycloalkyl are unsubstituted or substituted independently with 1, 2, 3, or 4 R²⁰ groups. In some embodiments, R²³ is

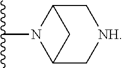 [SMILES: *N1C2CNCC1C2]

In some embodiments, R²³ is

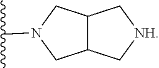 [SMILES: *N1CC2CNCC2C1]

In some embodiments of a compound of Formula (I) or (Ia), R²³ is

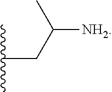 [SMILES: *CC(C)N]

In some embodiments, R²³

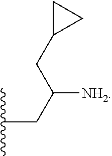 [SMILES: *CC(N)CC1CC1]

In some embodiments, R²³ is

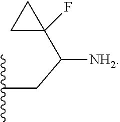 [SMILES: *CC(N)C1(F)CC1]

In some embodiments, R²³ is

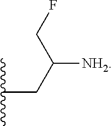 [SMILES: *CC(N)CF]

In some embodiments, R²³ is

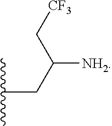 [SMILES: *CC(N)CC(F)(F)F]

In some embodiments, R²³ is

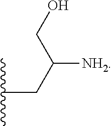 [SMILES: *CC(N)CO]

In some embodiments, R²³

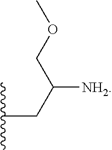 [SMILES: *CC(N)COC]

In some embodiments, R²³ is

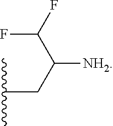 [SMILES: *CC(N)C(F)F]

In some embodiments, R²³ is

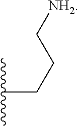 [SMILES: *CCCN]

In some embodiments of a compound of Formula (I) or (Ia), R²³ is —(C₁₋₆ alkylene)-C₃₋₁₀ cycloalkyl, —(C₁₋₆ alkylene)-4-10 membered heterocycloalkyl, —(C₁₋₆ heteroalkylene)-C₃₋₁₀ cycloalkyl, —(C₁₋₆ heteroalkylene)-4-10 membered heterocycloalkyl, —(C₁₋₆ alkylene)-C₆₋₁₀ aryl, —(C₁₋₆ alkylene)-5-10 membered heteroaryl, —(C₁₋₆ heteroalkylene)-C₆₋₁₀ aryl, —(C₁₋₆ heteroalkylene)-5-10 membered heteroaryl. In some embodiments, R²³ is —(C₁₋₆ alkylene)-C₃₋₁₀ cycloalkyl, optionally substituted with one or more R²⁰. In some embodiments, R²³ is —(C₁₋₆ alkylene)-4-10 membered heterocycloalkyl, optionally substituted with one or more R²⁰. In some embodiments, R²³ is —(C₁₋₆ heteroalkylene)-4-10 membered heterocycloalkyl, optionally substituted with one or more R²⁰. In some embodiments, R²³ is —(C₁₋₆ heteroalkylene)-C₃₋₁₀ cycloalkyl, optionally substituted with one or more R²⁰. In some embodiments, R²³ is —(C₁₋₆ alkylene)-C₆₋₁₀ aryl, optionally substituted with one or more R²⁰. In some embodiments, R²³ is —(C₁₋₆ alkylene)-5-10 membered heteroaryl, optionally substituted with one or more R²⁰. In some embodiments, R²³ is —(C₁₋₆ heteroalkylene)-C₆₋₁₀ aryl, optionally substituted with one or more R²⁰. In some embodiments, R²³ is —(C₁₋₆ heteroalkylene)-5-10 membered heteroaryl, optionally substituted with one or more R²⁰.

In some embodiments of a compound of Formula (I) or (Ia), R²³ is substituted or unsubstituted —(C₁₋₆ alkylene)-C₃₋₁₀ cycloalkyl. In some embodiments, R²³ is —(C₁₋₆ alkylene)-C₃₋₁₀ cycloalkyl, wherein —(C₁₋₆ alkylene)-C₃₋₁₀ cycloalkyl is substituted with 1, 2, or 3 independently selected R²⁰ groups. In some embodiments, the C₁₋₆ alkylene is C₁₋₃ alkylene. In some embodiments, the C₁₋₆ alkylene is CH₂. In some embodiments, the C₃₋₁₀ cycloalkyl is an optionally substituted a 3-6 membered ring. In some embodiments, the C₃₋₁₀ cycloalkyl is an optionally substituted a 3 membered ring. In some embodiments, the C₃₋₁₀ cycloalkyl is an optionally substituted a 4 membered ring. In some embodiments, the C₃₋₁₀ cycloalkyl is an optionally substituted a 5 membered ring. In some embodiments, the C₃₋₁₀ cycloalkyl is an optionally substituted a 6 membered ring. In some embodiments, the —C₃₋₁₀ cycloalkyl is

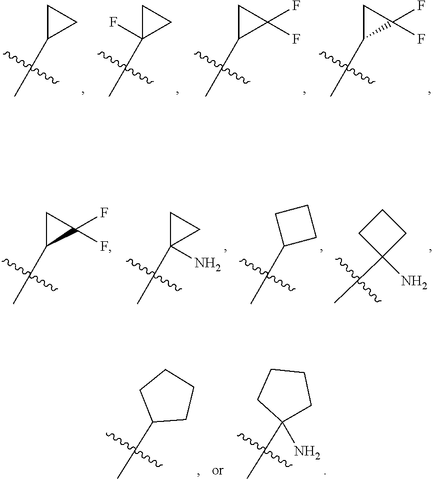

In some embodiments of a compound of Formula (I) or (Ia), R²³ is substituted or unsubstituted —(C₁₋₆ alkylene)-4-10 membered heterocycloalkyl. In some embodiments, R²³ is —(C₁₋₆ alkylene)-4-10 membered heterocycloalkyl, wherein —(C₁₋₆ alkylene)-4-10 membered heterocycloalkyl is substituted with 1, 2, or 3 independently selected R²⁰ groups. In some embodiments, the C₁₋₆ alkylene is C₁₋₃ alkylene. In some embodiments, the C₁₋₆ alkylene is CH₂. In some embodiments, the 4-10 membered heterocycloalkyl is an optionally substituted a 4-6 membered ring. In some embodiments, the 4-10 membered heterocycloalkyl is an optionally substituted a 4 membered ring. In some embodiments, the 4-10 membered heterocycloalkyl is an optionally substituted a 5 membered ring. In some embodiments, the 4-10 membered heterocycloalkyl is an optionally substituted a 6 membered ring. n some embodiments, the 4-10 membered heterocycloalkyl contains 0-1 oxygen and 0-2 nitrogen atoms. In some embodiments, the −4-10 membered heterocycloalkyl is

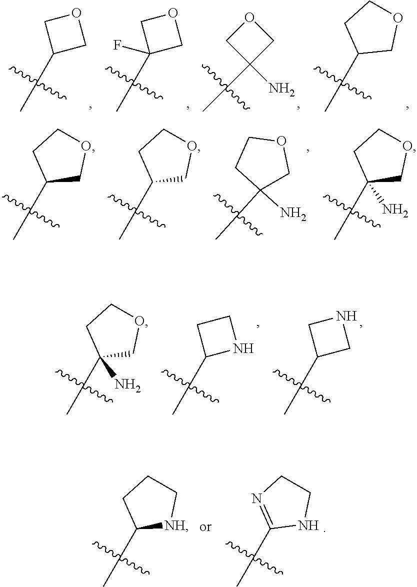

In some embodiments of a compound of Formula (I) or (Ia), R²³ is substituted or unsubstituted —(C₁₋₆ heteroalkylene)-C₃₋₁₀ cycloalkyl. In some embodiments, R²³ is —(C₁₋₆ heteroalkylene)-C₃₋₁₀ cycloalkyl, wherein —(C₁₋₆ heteroalkylene)-C₃₋₁₀ cycloalkyl is substituted with 1, 2, or 3 independently selected R²⁰ groups. In some embodiments, the heteroalkylene is C₁₋₃ heteroalkylene. In some embodiments, the C₃₋₁₀ cycloalkyl is an optionally substituted a 3-6 membered ring. In some embodiments, the C₃₋₁₀ cycloalkyl is an optionally substituted a 3 membered ring. In some embodiments, the C₃₋₁₀ cycloalkyl is an optionally substituted a 4 membered ring. In some embodiments, the C₃₋₁₀ cycloalkyl is an optionally substituted a 5 membered ring. In some embodiments, the C₃₋₁₀ cycloalkyl is an optionally substituted a 6 membered ring. In some embodiments, the heteroalkylene is C₁₋₃ heteroalkylene. In some embodiments, the —C₃₋₁₀ cycloalkyl is

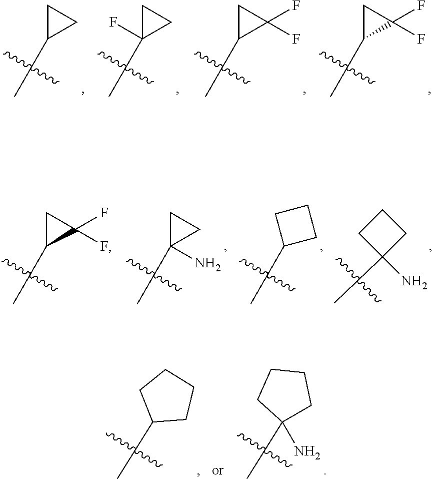

In some embodiments of a compound of Formula (I) or (Ia), R²³ is substituted or unsubstituted —(C₁₋₆ heteroalkylene)-4-10 membered heterocycloalkyl. In some embodiments, R²³ is —(C₁₋₆ heteroalkylene)-4-10 membered heterocycloalkyl, wherein —(C₁₋₆ heteroalkylene)-4-10 membered heterocycloalkyl is substituted with 1, 2, or 3 independently selected R²⁰ groups. In some embodiments, the heteroalkylene is C₁₋₃ heteroalkylene. In some embodiments, the 4-10 membered heterocycloalkyl is an optionally substituted a 4-6 membered ring. In some embodiments, the 4-10 membered heterocycloalkyl is an optionally substituted a 4 membered ring. In some embodiments, the 4-10 membered heterocycloalkyl is an optionally substituted a 5 membered ring. In some embodiments, the 4-10 membered heterocycloalkyl is an optionally substituted a 6 membered ring. n some embodiments, the 4-10 membered heterocycloalkyl contains 0-1 oxygen and 0-2 nitrogen atoms. In some embodiments of a compound of Formula (I) or (Ia), the −4-10 membered heterocycloalkyl is

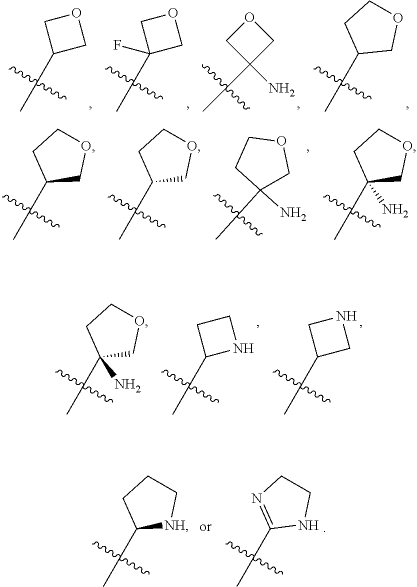

In some embodiments of a compound of Formula (I) or (Ia), R²³ is any one selected from the group consisting of:

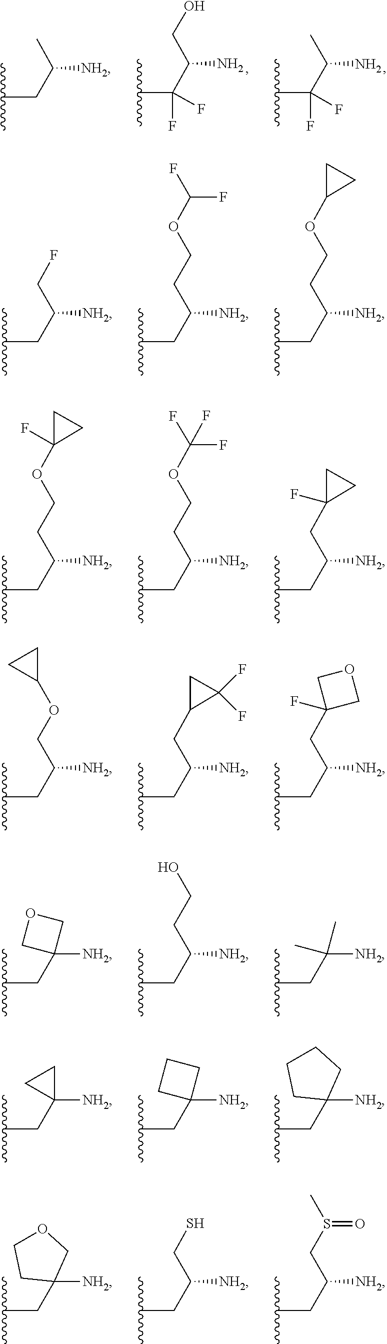

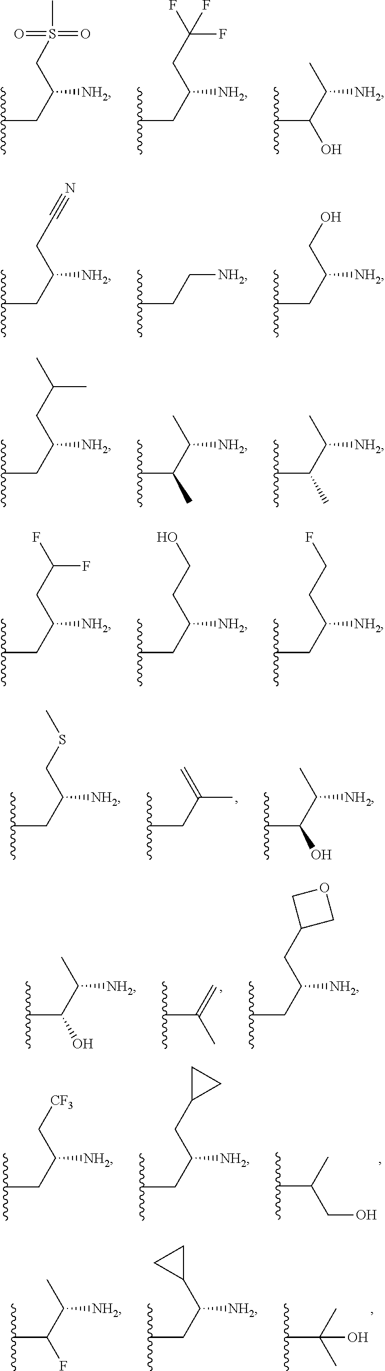

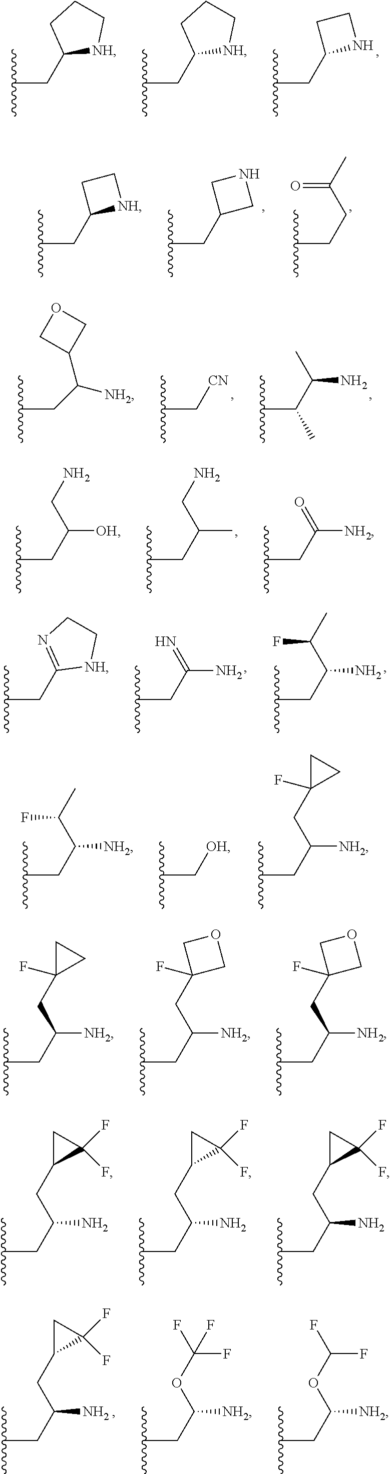

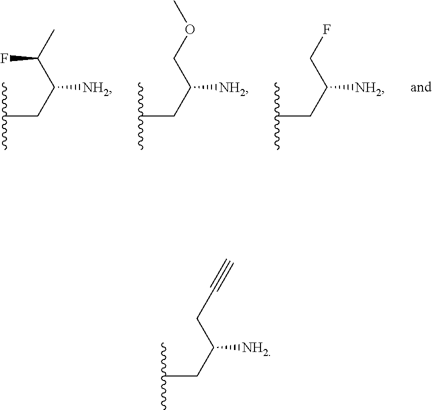

In some embodiments, R²³ is any one selected from the group consisting of:

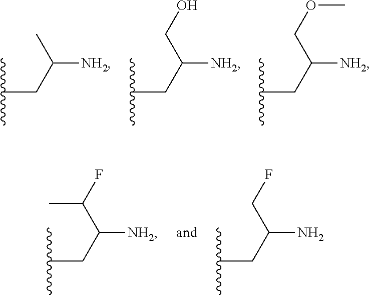

In some embodiments, R²³ is

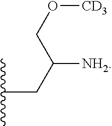 [SMILES: *CC(N)COC([2H])([2H])[2H]]

In some embodiments, R²³ is any one selected from the group consisting of:

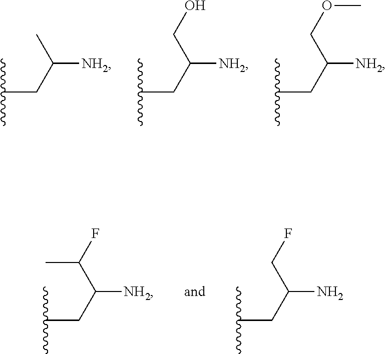

In some embodiments, R²³ is

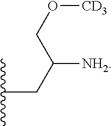 [SMILES: *CC(N)COC([2H])([2H])[2H]]

In some embodiments, R²³ is

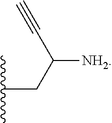 [SMILES: *CC(N)C#C]

In some embodiments, R²³ is

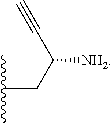 [SMILES: *C[C@@H](N)C#C]

In some embodiments, R²³ is

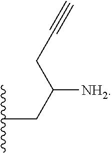 [SMILES: *CC(N)CC#C]

In some embodiments, R²³ is

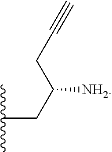 [SMILES: *C[C@@H](N)CC#C]

In some embodiments, R²³ is

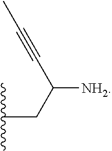 [SMILES: *CC(N)C#CC]

In some embodiments, R²³ is

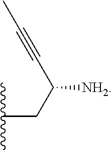 [SMILES: *C[C@@H](N)C#CC]

In some embodiments, R²³ is

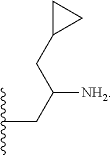 [SMILES: *CC(N)CC1CC1]

In some embodiments, R²³ is

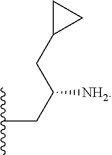 [SMILES: *C[C@@H](N)CC1CC1]

In some embodiments, R²³ is

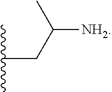 [SMILES: *CC(C)N]

In some embodiments, R²³ is

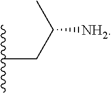 [SMILES: *C[C@H](C)N]

In some embodiments, R²³ is

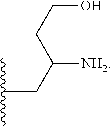 [SMILES: *CC(N)CCO]

In some embodiments, R²³ is

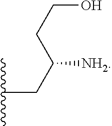 [SMILES: *C[C@@H](N)CCO]

In some embodiments, R²³ is

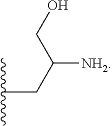 [SMILES: *CC(N)CO]

In some embodiments, R²³ is

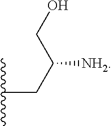 [SMILES: *C[C@@H](N)CO]

In some embodiments, R²³ is

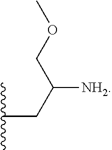 [SMILES: *CC(N)COC]

In some embodiments, R²³ is

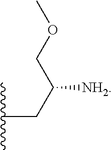 [SMILES: *C[C@@H](N)COC]

In some embodiments, R²³ is

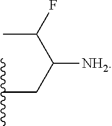 [SMILES: *CC(N)C(C)F]

In some embodiments, R²³ is

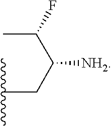 [SMILES: *C[C@@H](N)[C@H](C)F]

In some embodiments, R²³

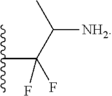 [SMILES: *C(F)(F)C(C)N]

In some embodiments, R²³ is

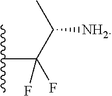 [SMILES: *C(F)(F)[C@H](C)N]

In some embodiments, R²³ is

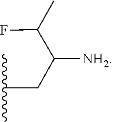 [SMILES: *CC(N)C(C)F]

In some embodiments, R²³ is

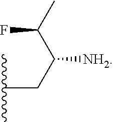 [SMILES: *C[C@@H](N)[C@H](C)F]

In some embodiments, R²³ is

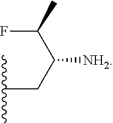 [SMILES: *C[C@@H](N)[C@H](C)F]

In some embodiments, R²³ is

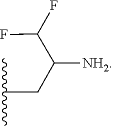 [SMILES: *CC(N)C(F)F]

In some embodiments, R²³ is

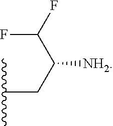 [SMILES: *C[C@@H](N)C(F)F]

In some embodiments, R²³ is

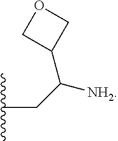 [SMILES: *CC(N)C1COC1]

In some embodiments, R²³ is

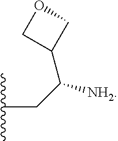

In some embodiments, R²³ is

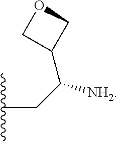

In some embodiments, R²³ is

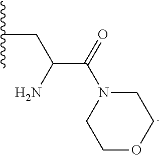 [SMILES: *CC(N)C(=O)N1CCOCC1]

In some embodiments, R²³ is

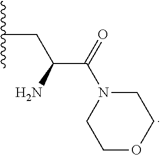 [SMILES: *C[C@H](N)C(=O)N1CCOCC1]

In some embodiments, R²³ is

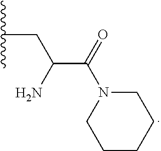 [SMILES: *CC(N)C(=O)N1CCCCC1]

In some embodiments, R²³ is

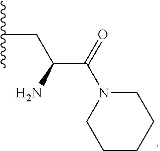 [SMILES: *C[C@H](N)C(=O)N1CCCCC1]

In some embodiments, R²³ is

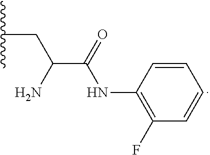 [SMILES: *CC(N)C(=O)Nc1ccccc1F]

In some embodiments, R²³ is

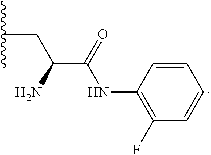 [SMILES: *C[C@H](N)C(=O)Nc1ccccc1F]

In some embodiments, R²³ is

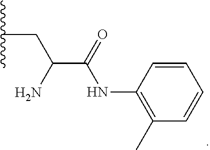 [SMILES: *CC(N)C(=O)Nc1ccccc1C]

In some embodiments, R²³ is

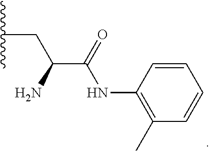 [SMILES: *C[C@H](N)C(=O)Nc1ccccc1C]

In some embodiments,

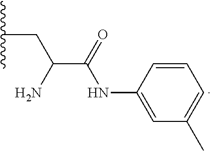 [SMILES: *CC(N)C(=O)Nc1cccc(C)c1]

### R²³ is

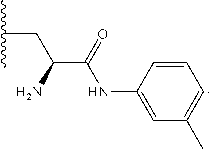 [SMILES: *C[C@H](N)C(=O)Nc1cccc(C)c1]

In some embodiments, R²³ is

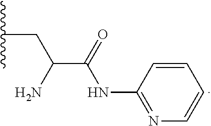 [SMILES: *CC(N)C(=O)Nc1ccccn1]

In some embodiments, R²³ is

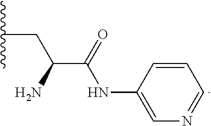 [SMILES: *C[C@H](N)C(=O)Nc1cccnc1]

In some embodiments, R²³ is

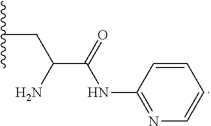 [SMILES: *CC(N)C(=O)Nc1ccccn1]

In some embodiments, R²³ is

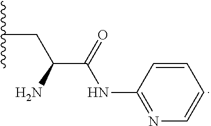 [SMILES: *C[C@H](N)C(=O)Nc1ccccn1]

In some embodiments, R²³ is

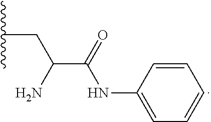 [SMILES: *CC(N)C(=O)Nc1ccccc1]

In some embodiments, R²³ is

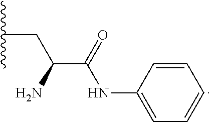 [SMILES: *C[C@H](N)C(=O)Nc1ccccc1]

In some embodiments, R²³ is

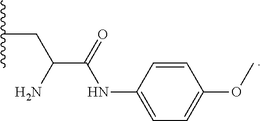 [SMILES: *CC(N)C(=O)Nc1ccc(OC)cc1]

In some embodiments, R²³ is

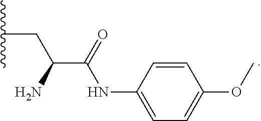 [SMILES: *C[C@H](N)C(=O)Nc1ccc(OC)cc1]

In some embodiments, R²³ is

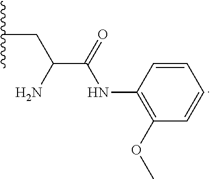 [SMILES: *CC(N)C(=O)Nc1ccccc1OC]

In some embodiments, R²³ is

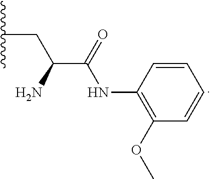 [SMILES: *C[C@H](N)C(=O)Nc1ccccc1OC]

In some embodiments, R²³ is

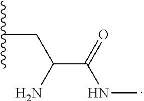 [SMILES: *CC(N)C(=O)NC]

In some embodiments, R²³ is

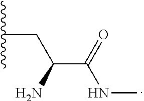 [SMILES: *C[C@H](N)C(=O)NC]

In some embodiments, R²³ is

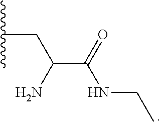 [SMILES: *CC(N)C(=O)NCC]

In some embodiments, R²³ is

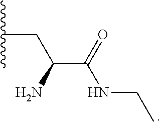 [SMILES: *C[C@H](N)C(=O)NCC]

In some embodiments, R²³ is

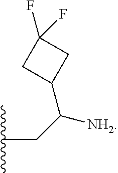 [SMILES: *CC(N)C1CC(F)(F)C1]

In some embodiments, R²³ is

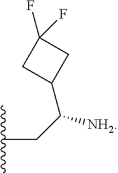 [SMILES: *C[C@@H](N)C1CC(F)(F)C1]

In some embodiments, R²³ is

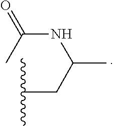 [SMILES: *CC(C)NC(C)=O]

In some embodiments, R²³ is

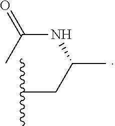 [SMILES: *C[C@@H](C)NC(C)=O]

In some embodiments, R²³ is

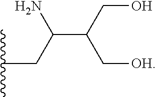 [SMILES: *CC(N)C(CO)CO]

In some embodiments, R²³ is

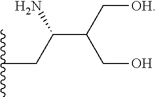 [SMILES: *C[C@H](N)C(CO)CO]

In some embodiments, R²³ is

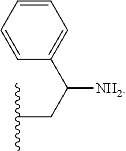 [SMILES: *CC(N)c1ccccc1]

In some embodiments, R²³ is

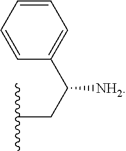 [SMILES: *C[C@@H](N)c1ccccc1]

In some embodiments, R²³ is

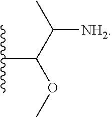 [SMILES: *C(OC)C(C)N]

In some embodiments, R²³ is

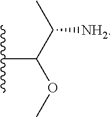 [SMILES: *C(OC)[C@H](C)N]

In some embodiments, R²³ is

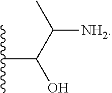 [SMILES: *C(O)C(C)N]

In some embodiments, R²³ is OH

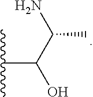 [SMILES: *C(O)[C@@H](C)N]

In some embodiments, R²³ is

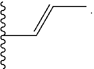 [SMILES: */C=C/C]

In some embodiments, R²³ is

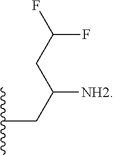 [SMILES: *CC(N)CC(F)F]

In some embodiments, R²³ is

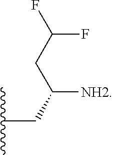 [SMILES: *C[C@@H](N)CC(F)F]

In some embodiments, R²³ is

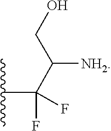 [SMILES: *C(F)(F)C(N)CO]

In some embodiments, R²³ is

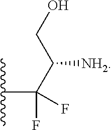 [SMILES: *C(F)(F)[C@@H](N)CO]

In some embodiments, R²³ is

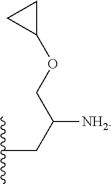 [SMILES: *CC(N)COC1CC1]

In some embodiments, R²³ is

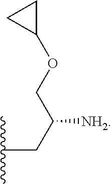 [SMILES: *C[C@@H](N)COC1CC1]

In some embodiments, R²³ is

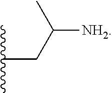 [SMILES: *CC(C)N]

In some embodiments, R²³ is

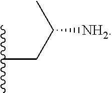 [SMILES: *C[C@H](C)N]

In some embodiments, R²³ is

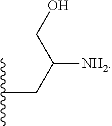 [SMILES: *CC(N)CO]

In some embodiments, R²³ is

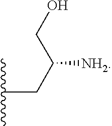 [SMILES: *C[C@@H](N)CO]

In some embodiments, R²³ is

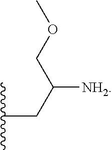 [SMILES: *CC(N)COC]

In some embodiments, R²³ is

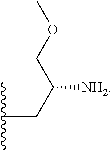 [SMILES: *C[C@@H](N)COC]

In some embodiments, R²³ is

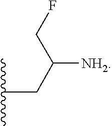 [SMILES: *CC(N)CF]

In some embodiments, R²³ is

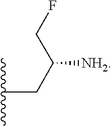 [SMILES: *C[C@@H](N)CF]

In some embodiments, R²³ is

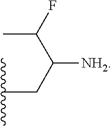 [SMILES: *CC(N)C(C)F]

In some embodiments, R²³ is

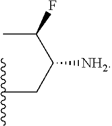 [SMILES: *C[C@@H](N)[C@@H](C)F]

In some embodiments, R²³ is

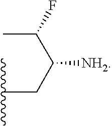 [SMILES: *C[C@@H](N)[C@H](C)F]

In some embodiments R²³ is

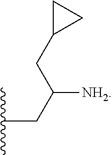 [SMILES: *CC(N)CC1CC1]

In some embodiments, R²³ is

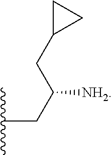 [SMILES: *C[C@@H](N)CC1CC1]

In some embodiments, R²³ is

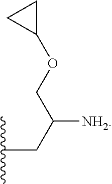 [SMILES: *CC(N)COC1CC1]

In some embodiments, R²³ is

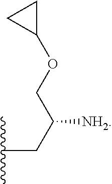 [SMILES: *C[C@@H](N)COC1CC1]

In some embodiments, R²³ is

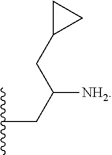 [SMILES: *CC(N)CC1CC1]

In some embodiments, R²³ is

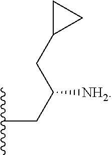 [SMILES: *C[C@@H](N)CC1CC1]

In some embodiments, R²³ is

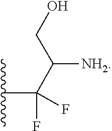 [SMILES: *C(F)(F)C(N)CO]

In some embodiments, R²³ is

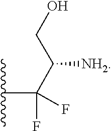 [SMILES: *C(F)(F)[C@@H](N)CO]

In some embodiments, R²³ is

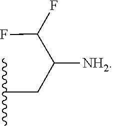 [SMILES: *CC(N)C(F)F]

In some embodiments, R²³ is

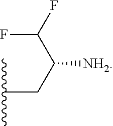 [SMILES: *C[C@@H](N)C(F)F]

In some embodiments, R²³ is

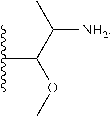 [SMILES: *C(OC)C(C)N]

In some embodiments, R²³ is

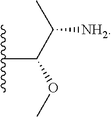 [SMILES: *[C@@H](OC)[C@H](C)N]

In some embodiments, R²³ is

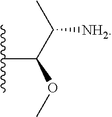 [SMILES: *[C@H](OC)[C@H](C)N]

In some embodiments, R²³ is

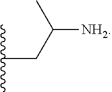 [SMILES: *CC(C)N]

In some embodiments, R²³ is

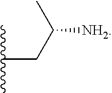 [SMILES: *C[C@H](C)N]

In some embodiments, R²³ is

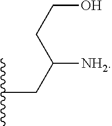 [SMILES: *CC(N)CCO]

In some embodiments, R²³ is

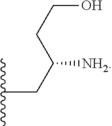 [SMILES: *C[C@@H](N)CCO]

In some embodiments, R²³ is

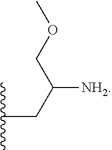 [SMILES: *CC(N)COC]

In some embodiments, R²³ is

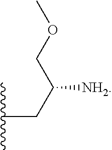 [SMILES: *C[C@@H](N)COC]

In some embodiments, R²³ is

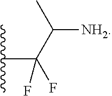 [SMILES: *C(F)(F)C(C)N]

In some embodiments, R²³ is

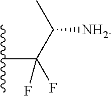 [SMILES: *C(F)(F)[C@H](C)N]

In some embodiments, R²³ is

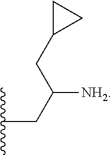 [SMILES: *CC(N)CC1CC1]

In some embodiments, R²³ is

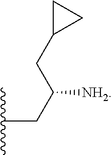 [SMILES: *C[C@@H](N)CC1CC1]

In some embodiments, R²³ is

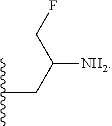 [SMILES: *CC(N)CF]

In some embodiments, R²³ is

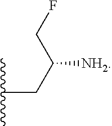 [SMILES: *C[C@@H](N)CF]

In some embodiments, R²³ is

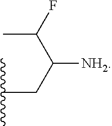 [SMILES: *CC(N)C(C)F]

In some embodiments, R²³ is

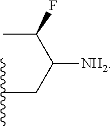 [SMILES: *CC(N)[C@@H](C)F]

In some embodiments, R²³ is

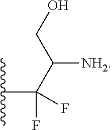 [SMILES: *C(F)(F)C(N)CO]

In some embodiments, R²³ is

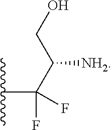 [SMILES: *C(F)(F)[C@@H](N)CO]

In some embodiments, R²³ is

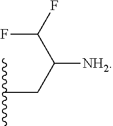 [SMILES: *CC(N)C(F)F]

In some embodiments, R²³ is

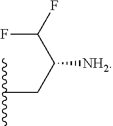 [SMILES: *C[C@@H](N)C(F)F]

In some embodiments, R²³ is

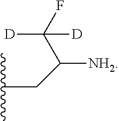 [SMILES: *CC(N)C([2H])([2H])F]

In some embodiments, R²³ is

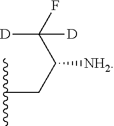 [SMILES: *C[C@@H](N)C([2H])([2H])F]

In some embodiments, R²³ is

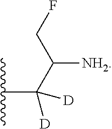 [SMILES: *C([2H])([2H])C(N)CF]

In some embodiments, R²³ is

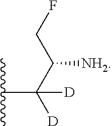 [SMILES: *C([2H])([2H])[C@@H](N)CF]

In some embodiments, R²³ is

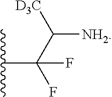 [SMILES: *C(F)(F)C(N)C([2H])([2H])[2H]]

In some embodiments, R²³ is

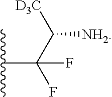 [SMILES: *C(F)(F)[C@@H](N)C([2H])([2H])[2H]]

In some embodiments, R²³ is

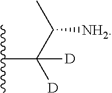 [SMILES: *C([2H])([2H])[C@H](C)N]

In some embodiments, R²³ is

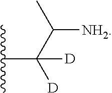 [SMILES: *C([2H])([2H])C(C)N]

In some embodiments, R²³ is

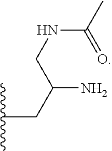 [SMILES: *CC(N)CNC(C)=O]

In some embodiments, R²³ is

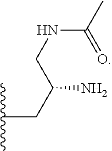 [SMILES: *C[C@@H](N)CNC(C)=O]

In some embodiments, R²³ is

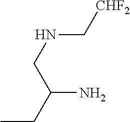

In some embodiments, R²³ is

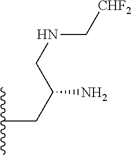 [SMILES: *C[C@@H](N)CNCC(F)F]

In some embodiments, R²³ is

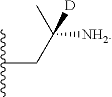 [SMILES: *C[C@]([2H])(C)N]

In some embodiments, R²³ is

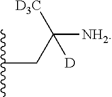 [SMILES: *CC([2H])(N)C([2H])([2H])[2H]]

In some embodiments, R²³ is

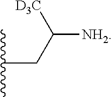 [SMILES: *CC(N)C([2H])([2H])[2H]]

In some embodiments, R²³ is

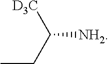

In some embodiments, R²³ is

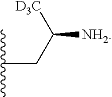 [SMILES: *C[C@H](N)C([2H])([2H])[2H]]

In some embodiments, R²³ is

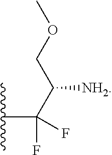 [SMILES: *C(F)(F)[C@@H](N)COC]

In some embodiments, R²³ is

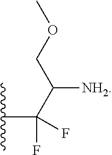 [SMILES: *C(F)(F)C(N)COC]

In some embodiments, R²³ is

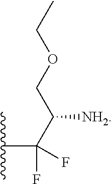 [SMILES: *C(F)(F)[C@@H](N)COCC]

In some embodiments, R²³ is

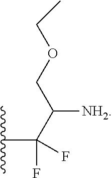 [SMILES: *C(F)(F)C(N)COCC]

In some embodiments, R²³ is

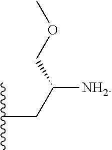 [SMILES: *C[C@@H](N)COC]

In some embodiments, R²³ is

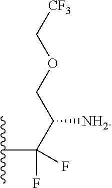 [SMILES: *C(F)(F)[C@@H](N)COCC(F)(F)F]

In some embodiments, R²³ is

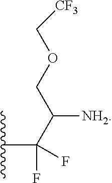 [SMILES: *C(F)(F)C(N)COCC(F)(F)F]

In some embodiments, R²³ is

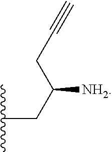 [SMILES: *C[C@H](N)CC#C]

In some embodiments, R²³ is

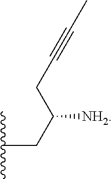 [SMILES: *C[C@@H](N)CC#CC]

In some embodiments, R²³ is

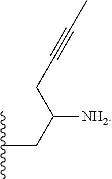 [SMILES: *CC(N)CC#CC]

In some embodiments, R²³ is

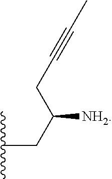 [SMILES: *C[C@H](N)CC#CC]

In some embodiments, R²³ is

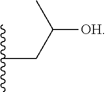 [SMILES: *CC(C)O]

In some embodiments, R²³ is

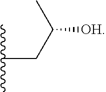 [SMILES: *C[C@H](C)O]

In some embodiments, R²³ is

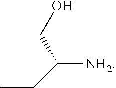

In some embodiments, R²³ is

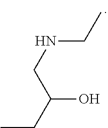

In some embodiments, R²³ is

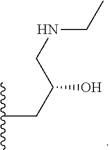 [SMILES: *C[C@@H](O)CNCC]

In some embodiments, R²³ is

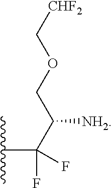 [SMILES: *C(F)(F)[C@@H](N)COCC(F)F]

In some embodiments, R²³ is

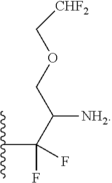 [SMILES: *C(F)(F)C(N)COCC(F)F]

In some embodiments, R²³ is

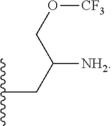 [SMILES: *CC(N)COC(F)(F)F]

In some embodiments, R²³ is

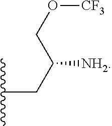 [SMILES: *C[C@@H](N)COC(F)(F)F]

In some embodiments, R²³ is

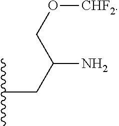 [SMILES: *CC(N)COC(F)F]

In some embodiments, R²³ is

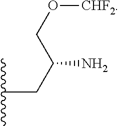 [SMILES: *C[C@@H](N)COC(F)F]

In some embodiments, R²³ is

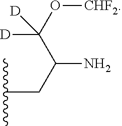 [SMILES: *CC(N)C([2H])([2H])OC(F)F]

In some embodiments, R²³ is

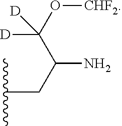 [SMILES: *CC(N)C([2H])([2H])OC(F)F]

In some embodiments, R²³ is

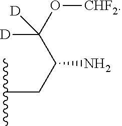 [SMILES: *C[C@@H](N)C([2H])([2H])OC(F)F]

In some embodiments, R²³ is

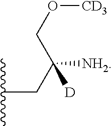 [SMILES: *C[C@@]([2H])(N)COC([2H])([2H])[2H]]

In some embodiments, R²³ is

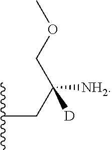 [SMILES: *C[C@@]([2H])(N)COC]

In some embodiments, R²³ is

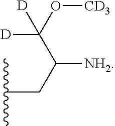 [SMILES: *CC(N)C([2H])([2H])OC([2H])([2H])[2H]]

In some embodiments, R²³ is

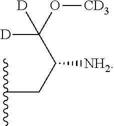 [SMILES: *C[C@@H](N)C([2H])([2H])OC([2H])([2H])[2H]]

In some embodiments, R²³ is

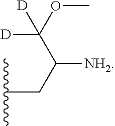 [SMILES: *CC(N)C([2H])([2H])OC]

In some embodiments, R²³ is

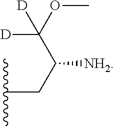 [SMILES: *C[C@@H](N)C([2H])([2H])OC]

In some embodiments, R²³ is

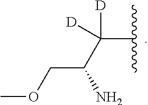 [SMILES: *C([2H])([2H])[C@H](N)COC]

In some embodiments, R²³ is

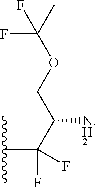

In some embodiments, R²³ is

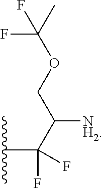 [SMILES: *C(F)(F)C(N)COC(C)(F)F]

In some embodiments, R²³ is

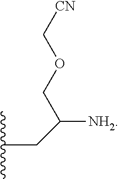 [SMILES: *CC(N)COCC#N]

In some embodiments, R²³ is

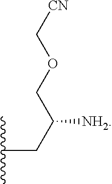 [SMILES: *C[C@@H](N)COCC#N]

In some embodiments, R²³ is

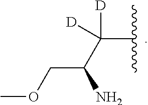 [SMILES: *C([2H])([2H])[C@@H](N)COC]

In some embodiments, R²³ is

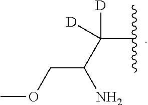 [SMILES: *C([2H])([2H])C(N)COC]

In some embodiments, R²³ is

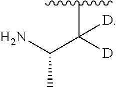

In some embodiments, R²³ is

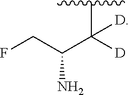

In some embodiments, R²³ is

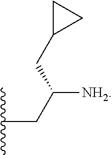 [SMILES: *C[C@@H](N)CC1CC1]

In some embodiments, R²³ is

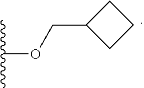 [SMILES: *OCC1CCC1]

In some embodiments, R²³ is

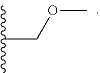 [SMILES: *COC]

In some embodiments, R²³ is

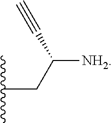 [SMILES: *C[C@@H](N)C#C]

In some embodiments, R²³ is

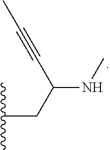 [SMILES: *CC(C#CC)NC]

In some embodiments, R²³ is

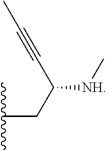 [SMILES: *C[C@H](C#CC)NC]

In some embodiments, R²³ is.

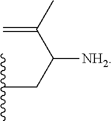 [SMILES: *CC(N)C(=C)C]

In some embodiments, R²³ is

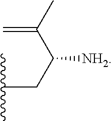 [SMILES: *C[C@@H](N)C(=C)C]

In some embodiments, R²³ is

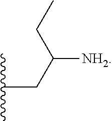 [SMILES: *CC(N)CC]

In some embodiments, R²³ is

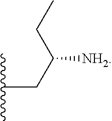 [SMILES: *C[C@@H](N)CC]

In some embodiments, R²³ is

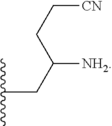 [SMILES: *CC(N)CCC#N]

In some embodiments R²³ is

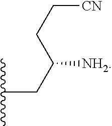 [SMILES: *C[C@@H](N)CCC#N]

In some embodiments, R²³ is

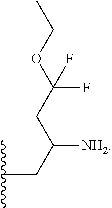 [SMILES: *CC(N)CC(F)(F)OCC]

In some embodiments, R²³ is

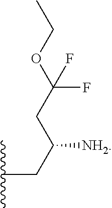 [SMILES: *C[C@@H](N)CC(F)(F)OCC]

In some embodiments, R²³ is

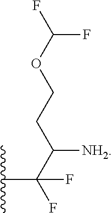 [SMILES: *C(F)(F)C(N)CCOC(F)F]

In some embodiments, R²³ is

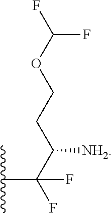 [SMILES: *C(F)(F)[C@@H](N)CCOC(F)F]

In some embodiments, R²³ is

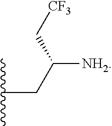 [SMILES: *C[C@@H](N)CC(F)(F)F]

In some embodiments, R²³ is

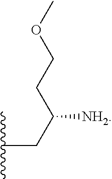 [SMILES: *C[C@@H](N)CCOC]

In some embodiments, R²³ is

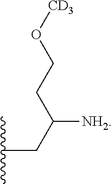 [SMILES: *CC(N)CCOC([2H])([2H])[2H]]

In some embodiments, R²³ is

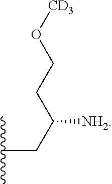 [SMILES: *C[C@@H](N)CCOC([2H])([2H])[2H]]

In some embodiments, R²³ is

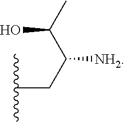 [SMILES: *C[C@@H](N)[C@H](C)O]

In some embodiments, R²³ is

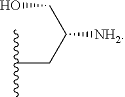

In some embodiments, R²³ is

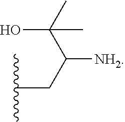 [SMILES: *CC(N)C(C)(C)O]

In some embodiments, R²³ is

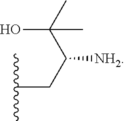 [SMILES: *C[C@@H](N)C(C)(C)O]

In some embodiments, R²³ is

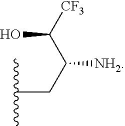 [SMILES: *C[C@@H](N)[C@@H](O)C(F)(F)F]

In some embodiments, R²³ is

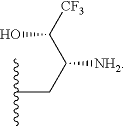 [SMILES: *C[C@@H](N)[C@H](O)C(F)(F)F]

In some embodiments, R²³ is

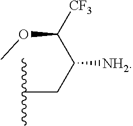 [SMILES: *C[C@@H](N)[C@@H](OC)C(F)(F)F]

In some embodiments, R²³ is

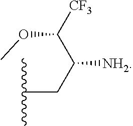 [SMILES: *C[C@@H](N)[C@H](OC)C(F)(F)F]

In some embodiments, R²³ is

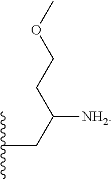 [SMILES: *CC(N)CCOC]

In some embodiments, R²³ is

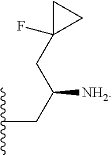 [SMILES: *C[C@H](N)CC1(F)CC1]

In some embodiments, R²³ is

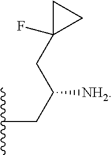 [SMILES: *C[C@@H](N)CC1(F)CC1]

In some embodiments, R²³ is

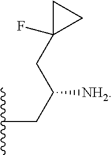 [SMILES: *C[C@@H](N)CC1(F)CC1]

In some embodiments, R²³ is

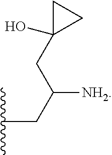 [SMILES: *CC(N)CC1(O)CC1]

In some embodiments, R²³ is

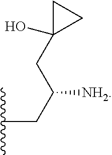 [SMILES: *C[C@@H](N)CC1(O)CC1]

In some embodiments, R²³ is

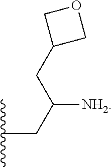 [SMILES: *CC(N)CC1COC1]

In some embodiments, R²³ is

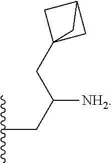 [SMILES: *CC(N)CC12CC(C1)C2]

In some embodiments, R²³ is

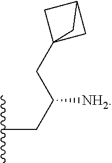 [SMILES: *C[C@@H](N)CC12CC(C1)C2]

In some embodiments, R²³ is

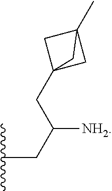 [SMILES: *CC(N)CC12CC(C)(C1)C2]

In some embodiments, R²³ is

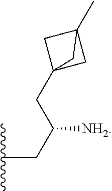 [SMILES: *C[C@@H](N)CC12CC(C)(C1)C2]

In some embodiments, R²³ is

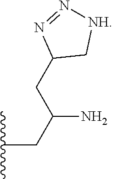 [SMILES: *CC(N)CC1CNN=N1]

In some embodiments, R²³ is

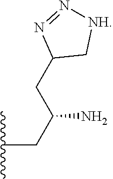 [SMILES: *C[C@@H](N)CC1CNN=N1]

In some embodiments, R²³ is

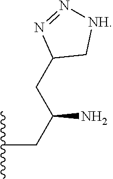 [SMILES: *C[C@H](N)CC1CNN=N1]

In some embodiments, R²³ is

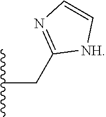 [SMILES: *Cc1ncc[nH]1]

In some embodiments, R²³ is

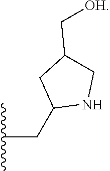 [SMILES: *CC1CC(CO)CN1]

In some embodiments, R²³ is

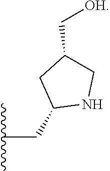 [SMILES: *C[C@@H]1C[C@H](CO)CN1]

In some embodiments, R²³ is

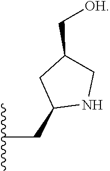 [SMILES: *C[C@H]1C[C@@H](CO)CN1]

In some embodiments, R²³ is

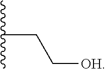 [SMILES: *CCO]

In some embodiments, R²³ is

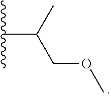 [SMILES: *C(C)COC]

In some embodiments, R²³ is

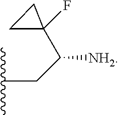 [SMILES: *C[C@@H](N)C1(F)CC1]

In some embodiments, R²³ is

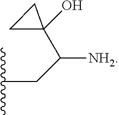 [SMILES: *CC(N)C1(O)CC1]

In some embodiments, R²³ is

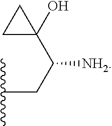 [SMILES: *C[C@@H](N)C1(O)CC1]

In some embodiments, R²³ is

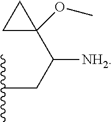 [SMILES: *CC(N)C1(OC)CC1]

In some embodiments, R²³ is

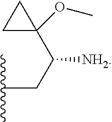 [SMILES: *C[C@@H](N)C1(OC)CC1]

In some embodiments, R²³ is

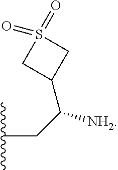 [SMILES: *C[C@@H](N)C1CS(=O)(=O)C1]

In some embodiments, R²³ is

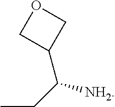

In some embodiments, R²³ is

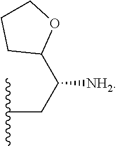 [SMILES: *C[C@@H](N)C1CCCO1]

In some embodiments, R²³ is

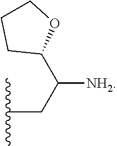 [SMILES: *CC(N)[C@@H]1CCCO1]

In some embodiments, R²³ is

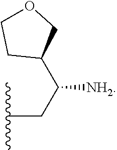 [SMILES: *C[C@@H](N)[C@H]1CCOC1]

In some embodiments, R²³ is

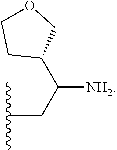 [SMILES: *CC(N)[C@@H]1CCOC1]

In some embodiments, R²³ is

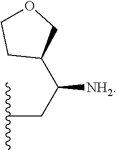 [SMILES: *C[C@H](N)[C@H]1CCOC1]

In some embodiments, R²³ is

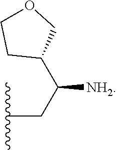 [SMILES: *C[C@H](N)[C@@H]1CCOC1]

In some embodiments, R²³ is

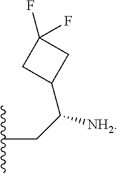 [SMILES: *C[C@@H](N)C1CC(F)(F)C1]

In some embodiments, R²³ is

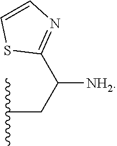 [SMILES: *CC(N)c1nccs1]

In some embodiments, R²³ is

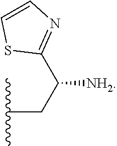 [SMILES: *C[C@@H](N)c1nccs1]

In some embodiments, R²³ is

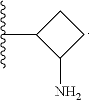 [SMILES: *C1CCC1N]

In some embodiments, R²³ is

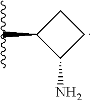 [SMILES: *[C@H]1CC[C@@H]1N]

In some embodiments, R²³ is

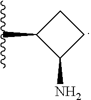 [SMILES: *[C@H]1CC[C@H]1N]

In some embodiments, R²³ is

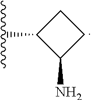 [SMILES: *[C@@H]1CC[C@H]1N]

In some embodiments, R²³ is

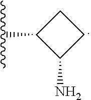 [SMILES: *[C@@H]1CC[C@@H]1N]

In some embodiments, R²³ is

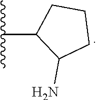 [SMILES: *C1CCCC1N]

In some embodiments, R²³ is

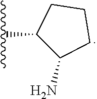 [SMILES: *[C@@H]1CCC[C@@H]1N]

In some embodiments, R²³ is

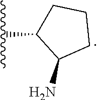 [SMILES: *[C@@H]1CCC[C@H]1N]

In some embodiments, R²³ is

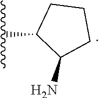 [SMILES: *[C@@H]1CCC[C@H]1N]

In some embodiments, R²³ is

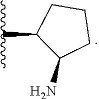 [SMILES: *[C@H]1CCC[C@H]1N]

In some embodiments, R²³ is

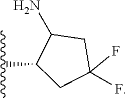 [SMILES: *[C@H]1CC(F)(F)CC1N]

In some embodiments, R²³ is

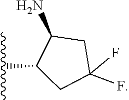 [SMILES: *[C@H]1CC(F)(F)C[C@@H]1N]

In some embodiments, R²³ is

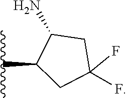 [SMILES: *[C@@H]1CC(F)(F)C[C@H]1N]

In some embodiments, R²³ is

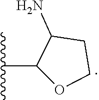 [SMILES: *C1OCCC1N]

In some embodiments R²³ is

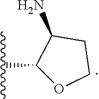 [SMILES: *[C@H]1OCC[C@@H]1N]

In some embodiments, R²³ is

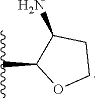 [SMILES: *[C@@H]1OCC[C@@H]1N]

In some embodiments, R²³ is

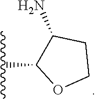 [SMILES: *[C@H]1OCC[C@H]1N]

In some embodiments, R²³ is

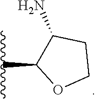 [SMILES: *[C@@H]1OCC[C@H]1N]

In some embodiments, R²³ is

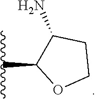 [SMILES: *[C@@H]1OCC[C@H]1N]

In some embodiments, R²³ is

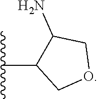 [SMILES: *C1COCC1N]

In some embodiments, R²³ is

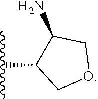 [SMILES: *[C@H]1COC[C@@H]1N]

In some embodiments, R²³ is

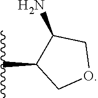 [SMILES: *[C@@H]1COC[C@@H]1N]

In some embodiments, R²³ is

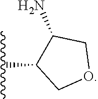 [SMILES: *[C@H]1COC[C@H]1N]

In some embodiments, R²³ is

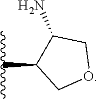 [SMILES: *[C@@H]1COC[C@H]1N]

In some embodiments, R²³ is

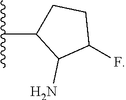 [SMILES: *C1CCC(F)C1N]

In some embodiments, R²³ is

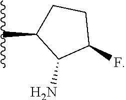 [SMILES: *[C@H]1CC[C@@H](F)[C@@H]1N]

In some embodiments, R²³ is

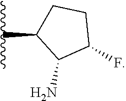 [SMILES: *[C@H]1CC[C@H](F)[C@@H]1N]

In some embodiments, R²³ is

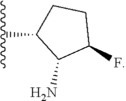 [SMILES: *[C@@H]1CC[C@@H](F)[C@@H]1N]

In some embodiments, R²³ is

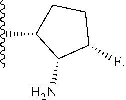 [SMILES: *[C@@H]1CC[C@H](F)[C@@H]1N]

In some embodiments, R²³ is

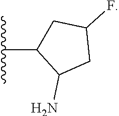 [SMILES: *C1CC(F)CC1N]

In some embodiments, R²³ is

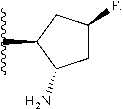 [SMILES: *[C@H]1C[C@@H](F)C[C@@H]1N]

In some embodiments, R²³ is

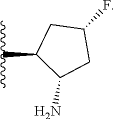 [SMILES: *[C@H]1C[C@H](F)C[C@@H]1N]

In some embodiments, R²³ is

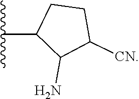 [SMILES: *C1CCC(C#N)C1N]

In some embodiments, R²³ is

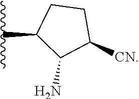 [SMILES: *[C@H]1CC[C@@H](C#N)[C@@H]1N]

In some embodiments, R²³ is

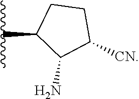 [SMILES: *[C@H]1CC[C@H](C#N)[C@@H]1N]

In some embodiments, R²³ is

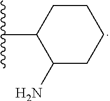 [SMILES: *C1CCCCC1N]

In some embodiments, R²³ is

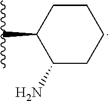 [SMILES: *[C@H]1CCCC[C@@H]1N]

In some embodiments, R²³ is

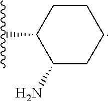 [SMILES: *[C@@H]1CCCC[C@@H]1N]

In some embodiments, R²³ is

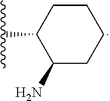 [SMILES: *[C@@H]1CCCC[C@H]1N]

In some embodiments, R²³ is

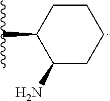 [SMILES: *[C@H]1CCCC[C@H]1N]

In some embodiments, R²³ is

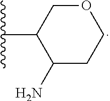 [SMILES: *C1COCCC1N]

In some embodiments, R²³ is

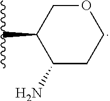 [SMILES: *[C@H]1COCC[C@@H]1N]

In some embodiments, R²³ is

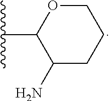 [SMILES: *C1OCCCC1N]

In some embodiments, R²³ is

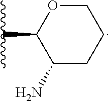 [SMILES: *[C@H]1OCCC[C@@H]1N]

In some embodiments, R²³ is

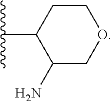 [SMILES: *C1CCOCC1N]

In some embodiments, R²³ is

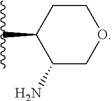 [SMILES: *[C@H]1CCOC[C@@H]1N]

In some embodiments, R²³ is

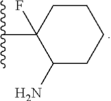 [SMILES: *C1(F)CCCCC1N]

In some embodiments, R²³ is

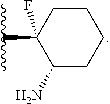 [SMILES: *[C@@]1(F)CCCC[C@@H]1N]

In some embodiments, R²³ is

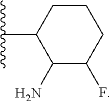 [SMILES: *C1CCCC(F)C1N]

In some embodiments, R²³ is

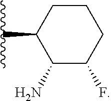 [SMILES: *[C@H]1CCC[C@H](F)[C@@H]1N]

In some embodiments, R²³ is

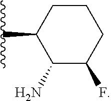 [SMILES: *[C@H]1CCC[C@@H](F)[C@@H]1N]

In some embodiments, R²³ is

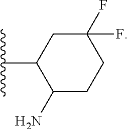 [SMILES: *C1CC(F)(F)CCC1N]

In some embodiments, R²³ is

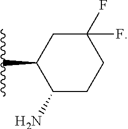 [SMILES: *[C@H]1CC(F)(F)CC[C@@H]1N]

In some embodiments, R²³ is

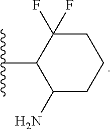 [SMILES: *C1C(N)CCCC1(F)F]

In some embodiments, R²³ is

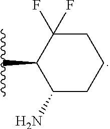 [SMILES: *[C@@H]1[C@@H](N)CCCC1(F)F]

In some embodiments, R²³ is

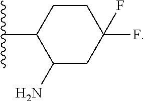 [SMILES: *C1CCC(F)(F)CC1N]

In some embodiments, R²³ is

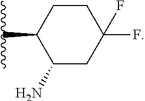 [SMILES: *[C@H]1CCC(F)(F)C[C@@H]1N]

In some embodiments of a compound of Formula (I) or (Ia), R²³ is methylene substituted with 1, 2, or 3 independently selected R²⁰ groups. In some embodiments, R²⁰ is methyl, ethyl, NH₂, CH₂OH, CH₂CH₂OH, CH₂CH₂F, CH₂CHF₂, or CH₂CH(CH₃)₂. In some embodiments, R²⁰ is NH₂ and methyl. In some embodiments, R²⁰ is NH₂ and CH₂OH. In some embodiments, R²⁰ is NH₂ and CH₂CH(CH₃)₂. In some embodiments, R²⁰ is NH₂ and CH₂CHF₂. In some embodiments, R²⁰ is NH₂ and CH₂CH₂F. In some embodiments, R²⁰ is NH₂ and CH₂CH₂OH. In some embodiments, R²⁰ is NH₂ and ethyl.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²⁰ is NH₂. In some embodiments, R²⁰ is OH. In some embodiments, R²⁰ is F. In some embodiments, R²⁰ is OCH₃. In some embodiments, R²⁰ is

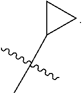 [SMILES: *C1CC1]

In some embodiments, R²⁰ is CH₂F. In some embodiments, R²⁰ is CHF₂. In some embodiments, R²⁰ is CF₃.

In some embodiments of a compound of Formula (I), (Ia), (IIa), (IIb), (IIc), (IId), (IIIa), (IIIb), (IIId), or (IIIe), X⁴ is CR²⁴. In some embodiments, R²⁴ is selected from the group consisting of —C≡CH, hydrogen, oxo, azido, halogen, —CN, —NO₂, C₁₋₆ haloalkyl, C₁₋₆ alkyl, C₂₋₆ alkenyl, C₁₋₆ heteroalkyl, —NR^{c4}C(═O)R^{b4}, —NR^{c4}C(═O)OR^{b4}, C₃₋₁₀ cycloalkyl, C₆₋₁₀ aryl, 5-10 membered heteroaryl, 4-10 membered heterocycloalkyl, —OR^{a4}, —NR^{c4}R^{d4} and —C(═O)NR^{c4}R^{d4}, wherein the C₁₋₆ alkyl, C₁₋₆ haloalkyl, C₂₋₆ alkenyl, C₁₋₆ heteroalkyl, C₃₋₁₀ cycloalkyl, C₆₋₁₀ aryl, 5-10 membered heteroaryl, and 4-10 membered heterocycloalkyl are unsubstituted or substituted independently with 1, 2, 3, or 4 R^{20d} groups. In some embodiments, R²⁴ is —OR^{a4}. In some embodiments, R²⁴ is OH. In some embodiments, R²⁴ is —OCH₃. In some embodiments, R²⁴ is C₁₋₆ alkyl. In some embodiments, R²⁴ is methyl. In some embodiments, R²⁴ is halo. In some embodiments, R²⁴ is fluoro, bromo, or chloro. In some embodiments, R²⁴ is F. In some embodiments, R²⁴ is Br. In some embodiments, R²⁴ is Cl. In some embodiments, R²⁴ is hydrogen. In some embodiments, R²⁴ is CN. In some embodiments, R²⁴ is C₃₋₁₀ cycloalkyl (e.g., cyclopropyl). In some embodiments, R²⁴ is phenyl. In some embodiments, R²⁴ is —NR^{c4}C(═O)R^{b4}. In some embodiments, R²⁴ is optionally substituted C₁₋₆ haloalkyl. In some embodiments, R²⁴ is optionally substituted 5-10 membered heteroaryl. In some embodiments, R²⁴ is optionally substituted 4-10 membered heterocycloalkyl. In some embodiments, R²⁴ is —C≡CH.

In some embodiments of a compound of Formula (I), (Ia), (IIa), (IIb), (IIc), (IId), (IIIa), (IIIb), (IIId), (IIIe), R²⁴ is selected from the group consisting of —C≡CH, 5-10 membered heteroaryl, and 4-10 membered heterocycloalkyl, wherein the 5-10 membered heteroaryl, and 4-10 membered heterocycloalkyl are unsubstituted or substituted independently with 1, 2, 3, or 4 R^{20d} groups. In some embodiments, R²⁴ is of —C≡CH. In some embodiments, R²⁴ is 5-10 membered heteroaryl. In some embodiments, R²⁴ is 5-10 membered heteroaryl substituted with 1 R^{20d} group. In some embodiments, R²⁴ is 5-10 membered heteroaryl substituted independently with 2 independently selected R^{20d} groups. In some embodiments, R²⁴ is 5-10 membered heteroaryl substituted independently with 3 independently selected R^{20d} groups. In some embodiments, R²⁴ is 5-10 membered heteroaryl substituted independently with 4 independently selected R^{20d} groups. In some embodiments, R²⁴ is 4-10 membered heterocycloalkyl. In some embodiments, R²⁴ is 4-10 membered heterocycloalkyl substituted with 1 R^{20d} group. In some embodiments, R²⁴ is 4-10 membered heterocycloalkyl with 2 independently selected R^{20d} groups. In some embodiments, R²⁴ is 4-10 membered heterocycloalkyl with 3 independently selected R^{20d} groups. In some embodiments, R²⁴ is 4-10 membered heterocycloalkyl with 4 independently selected R^{20d} groups.

In some embodiments of a compound of Formula (I), (Ia), (Ia), (IIb), (IIc), (IId), (IIIa), (IIIb), (IIId), or (IIIe), R²⁴ is

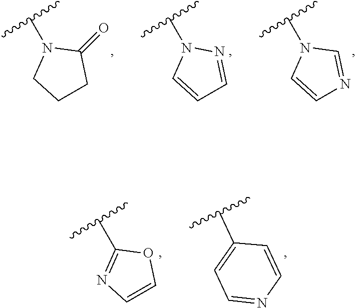 [SMILES: *N1CCCC1=O ; *n1cccn1 ; *n1ccnc1 ; *c1ncco1 ; *c1ccncc1]

In some embodiments, R²⁴ is

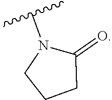 [SMILES: *N1CCCC1=O]

In some embodiments, R²⁴ is

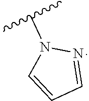 [SMILES: *n1cccn1]

In some embodiments, R²⁴ is

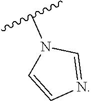 [SMILES: *n1ccnc1]

In some embodiments, R²⁴ is

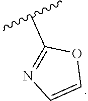 [SMILES: *c1ncco1]

In some embodiments, R²⁴ is

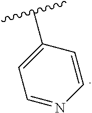 [SMILES: *c1ccncc1]

In some embodiments, R²⁴ is

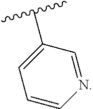 [SMILES: *c1cccnc1]

In some embodiments, R²⁴ is

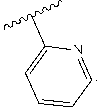 [SMILES: *c1ccccn1]

In some embodiments, R²⁴ is

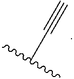 [SMILES: *C#C]

In some embodiments of a compound of Formula (I), (Ia), (IIa), (IIb), (IIc), (IId), (IIIa), (IIIb), (IIId), or (IIIe), R²⁴ is H, CH₃, Cl, Br, CF₃,

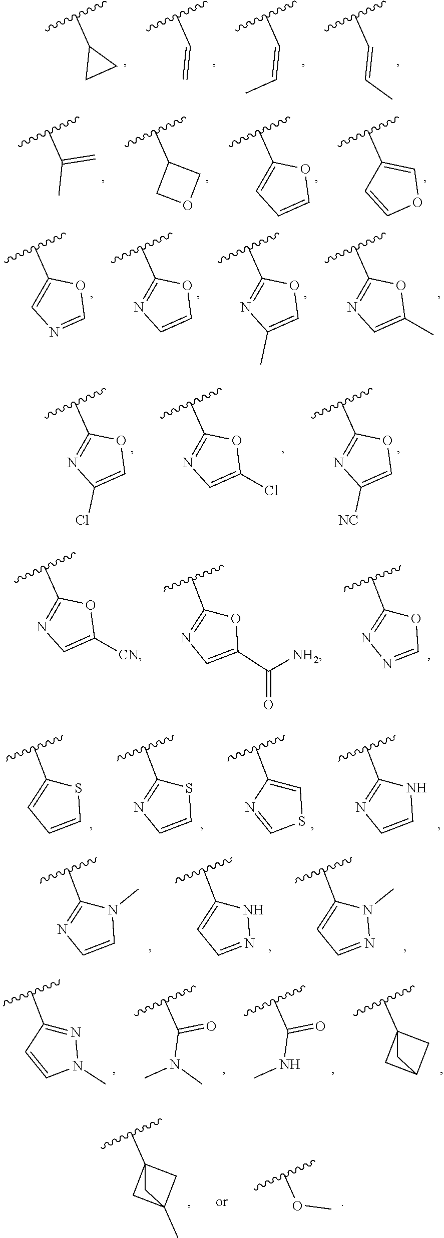

In some embodiments, R²⁴ is H. In some embodiments, R²⁴ is CH₃. In some embodiments, R²⁴ is Cl. In some embodiments, R²⁴ is Br. In some embodiments, R²⁴ is CF₃. In some embodiments, R²⁴ is

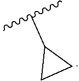 [SMILES: *C1CC1]

In some embodiments, R²⁴ is

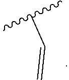 [SMILES: *C=C]

In some embodiments, R²⁴ is

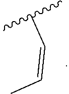 [SMILES: */C=C\C]

In some embodiments, R²⁴ is

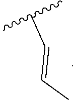 [SMILES: */C=C/C]

In some embodiments, R²⁴ is

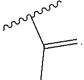 [SMILES: *C(=C)C]

In some embodiments, R²⁴ is

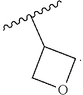 [SMILES: *C1COC1]

In some embodiments, R²⁴ is

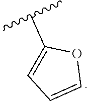 [SMILES: *c1ccco1]

In some embodiments, R²⁴ is

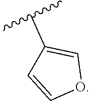 [SMILES: *c1ccoc1]

In some embodiments, R²⁴ is

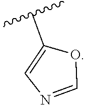 [SMILES: *c1cnco1]

In some embodiments, R²⁴ is

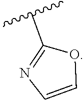 [SMILES: *c1ncco1]

In some embodiments, R²⁴ is

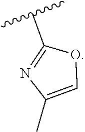 [SMILES: *c1nc(C)co1]

In some embodiments, R²⁴ is

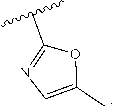 [SMILES: *c1ncc(C)o1]

In some embodiments, R²⁴ is

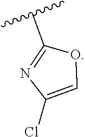 [SMILES: *c1nc(Cl)co1]

In some embodiments, R²⁴ is

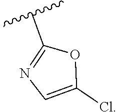 [SMILES: *c1ncc(Cl)o1]

In some embodiments, R²⁴ is

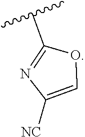 [SMILES: *c1nc(C#N)co1]

In some embodiments, R²⁴ is

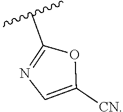 [SMILES: *c1ncc(C#N)o1]

In some embodiments, R²⁴ is

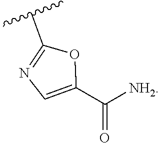 [SMILES: *c1ncc(C(N)=O)o1]

In some embodiments, R²⁴ is

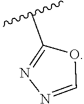 [SMILES: *c1nnco1]

In some embodiments, R²⁴ is

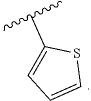 [SMILES: *c1cccs1]

In some embodiments, R²⁴ is

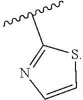 [SMILES: *c1nccs1]

In some embodiments, R²⁴ is

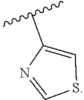 [SMILES: *c1cscn1]

In some embodiments, R²⁴ is

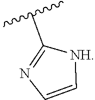 [SMILES: *c1ncc[nH]1]

In some embodiments, R²⁴ is

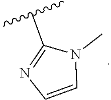 [SMILES: *c1nccn1C]

In some embodiments, R²⁴ is

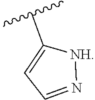 [SMILES: *c1ccn[nH]1]

In some embodiments, R²⁴ is

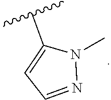 [SMILES: *c1ccnn1C]

In some embodiments, R²⁴ is

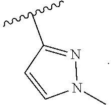 [SMILES: *c1ccn(C)n1]

In some embodiments, R²⁴ is

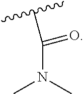 [SMILES: *C(=O)N(C)C]

In some embodiments, R²⁴ is

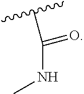 [SMILES: *C(=O)NC]

In some embodiments, R²⁴ is

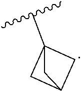 [SMILES: *C12CC(C1)C2]

In some embodiments, R²⁴ is

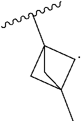 [SMILES: *C12CC(C)(C1)C2]

In some embodiments, R²⁴ is

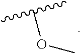 [SMILES: *OC]

In some embodiments of a compound of Formula (I), (Ia), (IIa), (IIb), (IIc), (IId), (IIIa), (IIIb), (IIId), or (IIIe), each R^{20d} is independently selected from the group consisting of —OH, —SH, —CN, —NO₂, halogen, oxo, amino, carbamyl, carbamoyl, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments of a compound of Formula (I), (Ia), (IIa), (IIb), (IIc), (IId), (IIIa), (IIIb), (IIId), or (IIIe), R^{20d} is —OH. In some embodiments, R^{20d} is —SH. In some embodiments, R^{20d} is —CN. In some embodiments, R^{20d} is —NO₂. In some embodiments, R^{20d} is halogen. In some embodiments, R^{20d} is oxo. In some embodiments, R^{20d} is C₁₋₄ alkyl. In some embodiments, R^{20d} is C₂₋₄ alkenyl. In some embodiments, R^{20d} is C₁₋₄ haloalkyl. In some embodiments, R^{20d} is C₁₋₄ cyanoalkyl. In some embodiments, R^{20d} is C₁₋₄ hydroxyalkyl. In some embodiments, R^{20d} is C₁₋₄ alkoxy, —(C₁₋₄ alkyl)-(C₁₋₄ alkoxy), —(C₁₋₄ alkoxy)-(C₁₋₄ alkoxy). In some embodiments, R^{20d} is C₁₋₄ haloalkoxy. In some embodiments, R^{20d} is C₃₋₆ cycloalkyl. In some embodiments, R^{20d} is phenyl. In some embodiments, R^{20d} is 5-6 membered heteroaryl. In some embodiments, R^{20d} is 4-6 membered heterocycloalkyl. In some embodiments, R^{20d} is amino. In some embodiments, R^{20d} is C₁₋₄ alkylamino. In some embodiments, R^{20d} is di(C₁₋₄ alkyl)amino. In some embodiments, R^{20d} is carbamyl. In some embodiments, R^{20d} is C₁₋₄ alkylcarbamyl. In some embodiments, R^{20d} is di(C₁₋₄ alkyl)carbamyl. In some embodiments, R^{20d} is carbamoyl. In some embodiments, R^{20d} is C₁₋₄ alkylcarbamoyl. In some embodiments, R^{20d} is di(C₁₋₄ alkyl)carbamoyl. In some embodiments, R^{20d} is C₁₋₄ alkylcarbonyl. In some embodiments, R^{20d} is C₁₋₄ alkoxycarbonyl. In some embodiments, R^{20d} is C₁₋₄ alkylcarbonylamino. In some embodiments, R^{20d} is C₁₋₄ alkylsulfonylamino. In some embodiments, R^{20d} is aminosulfonyl. In some embodiments, R^{20d} is C₁₋₄ alkylaminosulfonyl. In some embodiments, R^{20d} is di(C₁₋₄ alkyl)aminosulfonyl. In some embodiments, R^{20d} is aminosulfonylamino. In some embodiments, R^{20d} is C₁₋₄ alkylaminosulfonylamino. In some embodiments, R^{20d} is di(C₁₋₄ alkyl)aminosulfonylamino. In some embodiments, R^{20d} is aminocarbonylamino. In some embodiments, R^{20d} is C₁₋₄ alkylaminocarbonylamino. In some embodiments, R^{20d} is di(C₁₋₄ alkyl)aminocarbonylamino.

In some embodiments of a compound of Formula (I), (Ia), (IIa), (IIb), (IIc), (IId), (IIIa), (IIIb), (IIId), or (IIIe), each R^{20d} independently selected from the group consisting of —OH, —SH, —CN, —NO₂, halogen, oxo, amino, carbamyl, carbamoyl, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein alkyl, alkenyl, heteroalkyl, cycloalkyl, phenyl, heteroaryl and heterocycloalkyl are each optionally substituted with 1, 2, 3 or 4 R³² groups, wherein each R³² is independently —OH, —SH, —CN, —NO₂, halogen, oxo, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy. In some embodiments, R³² is —OH. In some embodiments, R³² is —SH. In some embodiments, R³² is —CN. In some embodiments, R³² is —NO₂. In some embodiments, R³² is halogen. In some embodiments, R³² is oxo. In some embodiments, R³² is C₁₋₄ alkyl. In some embodiments, R³² is C₂₋₄ alkenyl. In some embodiments, R³² is C₁₋₄ haloalkyl. In some embodiments, R³² is C₁₋₄ cyanoalkyl. In some embodiments, R³² is C₁₋₄ hydroxyalkyl. In some embodiments, R³² is C₁₋₄ alkoxy.

In some embodiments of a compound of Formula (I), (Ia), (IIa), (IIb), (IIc), (IId), (IIIa), (IIIb), (IIId), or (IIIe), each R^{20d} independently selected from the group consisting of —OH, —SH, —CN, —NO₂, halogen, oxo, amino, carbamyl, carbamoyl, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein alkyl, alkenyl, heteroalkyl, cycloalkyl, phenyl, heteroaryl and heterocycloalkyl are each optionally substituted with 1, 2, 3 or 4 R³² groups, wherein each R³² is independently oxo, halogen, methyl, ethyl, —CN, —CF₃, —OH, —OMe, —NH₂, or —NO₂. In some embodiments, R³² is oxo. In some embodiments, R³² halogen. In some embodiments, R³² methyl, ethyl. In some embodiments, R³², —CN. In some embodiments, R³², —CF₃. In some embodiments, R³², —OH. In some embodiments, R³², —OMe. In some embodiments, R³², —NH₂. In some embodiments, R³²—NO₂.

In some embodiments, the compound is of the Formula (IIa):

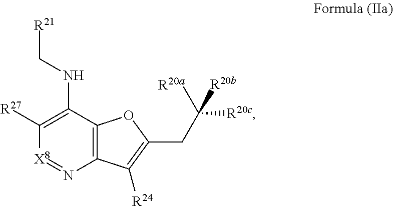

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20c} is NH₂. In some embodiments, R^{20b} is hydrogen.

In some embodiments, the compound is of the Formula (IIb):

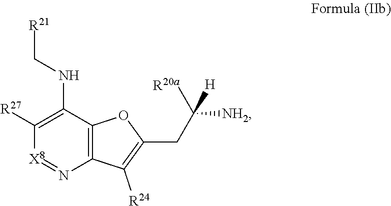

wherein R^{20a} is selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂.

In some embodiments, the compound is of Formula (IIc):

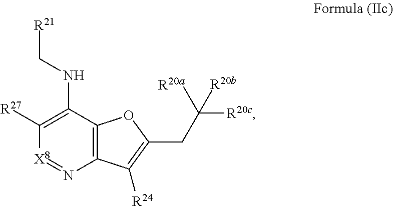

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkyl carb onyl amino, C₁₋₄ alkyl sulfonyl amino, aminosulfonyl, C₁₋₄ alkylaminosulfonyl, di(C₁₋₄ alkyl)aminosulfonyl, aminosulfonyl amino, C₁₋₄ alkyl aminosulfonyl amino, di(C₁₋₄ alkyl)aminosulfonylamino, aminocarb onyl amino, C₁₋₄ alkyl aminocarb onyl amino, and di(C₁₋₄ alkyl)aminocarb onyl amino, or a pharmaceutically acceptable salt thereof.

In some embodiments, the compound is of Formula (IId):

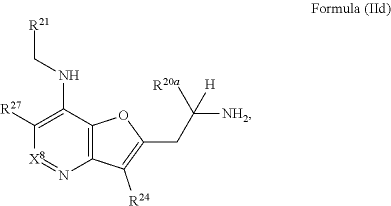

wherein R^{20a} is selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, the compound is of the Formula (IIIa):

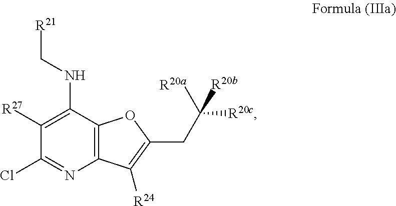

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20c}, is NH₂. In some embodiments, R^{20b} is hydrogen.

In some embodiments, the compound is of the Formula (IIIb):

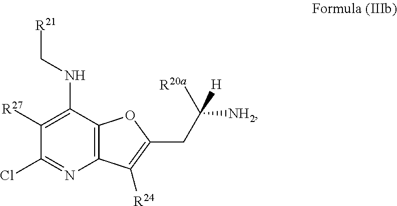

wherein R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂.

In some embodiments of a compound of Formula (I), (Ia), (IIa), (IIb), (IIc), (IId), (IIIa), (IIIb), (IIId), or (IIIe), R²⁴ is C₁₋₆ alkyl. In some embodiments, R²⁴ is methyl. In some embodiments, R²⁴ is halo. In some embodiments, R²⁴ is fluoro, bromo, or chloro. In some embodiments, R²⁴ is hydrogen. In some embodiments, R²⁴ is CN. In some embodiments, R²⁴ is C₃₋₁₀ cycloalkyl.

In some embodiments, the compound is of the Formula (IIIc):

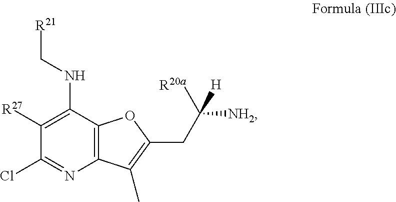

wherein R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂.

In some embodiments, the compound is of Formula (IIId):

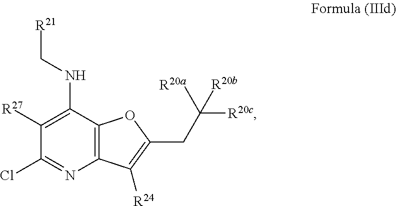

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, the compound is of Formula (IIIe):

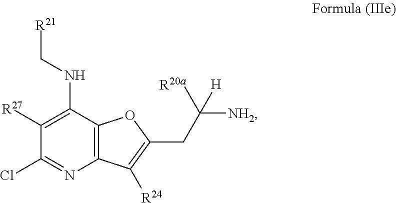

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, the compound is of the Formula (IIIf):

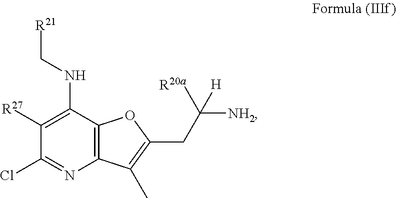

wherein R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, the compound is of the Formula (IIIg):

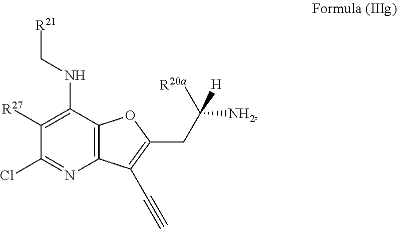

wherein R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂.

In some embodiments, the compound is of the Formula (IIIh):

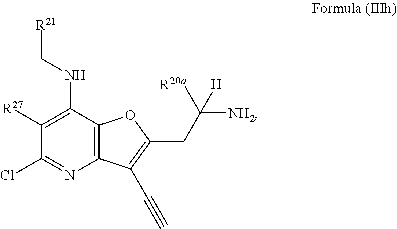

wherein R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, or a pharmaceutically acceptable salt thereof.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²⁷ is selected from the group consisting of H, azido, halo, C₁₋₆ alkyl, C₂₋₆ alkenyl, C₂₋₆ alkynyl, C₁₋₆ heteroalkyl, —CN, —NO₂, —OR^{a7}, —C(═O)R^{b7}, —C(═O)OR^{b7}, —NR^{c7}R^{d7}, —C(═O)NR^{c7}R^{d7}, —OC(═O)NR^{c7}R^{d7}, —NR^{c7}C(═O)R^{b7}, —NR^{c7}C(═O)OR^{b7}, —NR^{c7}C(═O)NR^{c7}R^{d7}, —NR^{c7}S(═O)₂R^{b7}, and —NR^{c7}S(═O)₂NR^{c7}R^{d7}, wherein the C₁₋₆ alkyl, C₁₋₆ heteroalkyl, C₂₋₆ alkenyl, and C₂₋₆ alkynyl are each unsubstituted or substituted with 1, 2, 3, or 4 independently selected R²⁰ groups. In some embodiments, R²⁷ is C₁₋₆ alkyl substituted with 1, 2, 3, or 4 independently selected R²⁰ groups. In some embodiments, R²⁷ is C₁₋₆ heteroalkyl substituted with 1, 2, 3, or 4 independently selected R²⁰ groups. In some embodiments, R²⁷ is C₂₋₆ alkenyl substituted with 1, 2, 3, or 4 independently selected R²⁰ groups. In some embodiments, R²⁷ is C₂₋₆ alkynyl substituted with 1, 2, 3, or 4 independently selected R²⁰ groups.

In some embodiments of a compound of Formula (I), (Ia), (IIa), (IIb), (IIc), (IId), (IIIa), (IIIb), (IIIc), (IIId), (IIIe), (IIIf), (IIIg), or (IIIh), R²⁷ is selected from the group consisting of H, azido, halo, C₁₋₆ alkyl, C₂₋₆ alkenyl, C₂₋₆ alkynyl, C₁₋₆ heteroalkyl, —CN, —NO₂, —OR^{a7}, —C(═O)R^{b7}, —C(═O)OR^{b7}, —NR^{c7}R^{d7}, —C(═O)NR^{c7}R^{d7}, —OC(═O)NR^{c7}R^{d7}, —NR^{c7}C(═O)R^{b7}, —NR^{c7}C(═O)OR^{b7}, —NR^{c7}C(═O)NR^{c7}R^{d7}, —NR^{c7}S(═O)₂R^{b7}, and —NR^{c7}S(═O)₂NR^{c7}R^{d7}. In some embodiments, R²⁷ is H. In some embodiments, R²⁷ is azido. In some embodiments, R²⁷ is halo. In some embodiments, R²⁷ is C₁₋₆ alkyl. In some embodiments, R²⁷ is C₂₋₆ alkenyl. In some embodiments, R²⁷ is C₂₋₆ alkynyl. In some embodiments, R²⁷ is C₁₋₆ heteroalkyl. In some embodiments, R²⁷ is —CN. In some embodiments, R²⁷ is —NO₂. In some embodiments, R²⁷ is —OR^{a7}. In some embodiments, R²⁷ is —C(═O)R^{b7}. In some embodiments, R²⁷ is —C(═O)OR^{b7}. In some embodiments, R²⁷ is —NR^{c7}R^{d7}. In some embodiments, R²⁷ is —C(═O)NR^{c7}R^{d7}. In some embodiments, R²⁷ is —OC(═O)NR^{c7}R^{d7}. In some embodiments, R²⁷ is —NR^{c7}C(═O)R^{b7}. In some embodiments, R²⁷ is —NR^{c7}C(═O)OR^{b7}. In some embodiments, R²⁷ is —NR^{c7}C(═O)NR^{c7}R^{d7}. In some embodiments, R²⁷ is —NR^{c7}S(═O)₂R^{b7}. In some embodiments, R²⁷ is —NR^{c7}S(═O)₂NR^{c7}R^{d7}.

In some embodiments of a compound of (I), (Ia), (IIa), (IIb), (IIc), (IId), (IIIa), (IIIb), (IIIc), (IIId), (IIIe), (IIIf), (IIIg), or (IIIh), R²⁷ is H or F. In some embodiments, R²⁷ is F.

In some embodiments of a compound of Formula (I), (Ia), (IIa), (IIb), (IIc), or (IId), R²⁸ is selected from the group consisting of H, azido, halo, C₁₋₆ alkyl, C₂₋₆ alkenyl, C₂₋₆ alkynyl, C₁₋₆ heteroalkyl, —CN, —NO₂, —OR^{a7}, —C(═O)R^{b7}, —C(═O)OR^{b7}, —NR^{c7}R^{d7}, —C(═O)NR^{c7}R^{d7}, —OC(═O)NR^{c7}R^{d7}, —NR^{c7}C(═O)R^{b7}, —NR^{c7}C(═O)OR^{b7}, —NR^{c7}C(═O)NR^{c7}R^{d7}, —NR^{c7}S(═O)₂R^{b7}, and —NR^{c7}S(═O)₂NR^{c7}R^{d7}, wherein the C₁₋₆ alkyl, C₁₋₆ heteroalkyl, C₂₋₆ alkenyl, and C₂₋₆ alkynyl are each unsubstituted or substituted with 1, 2, 3, or 4 independently selected R²⁰ groups. In some embodiments, R²⁸ is C₁₋₆ alkyl substituted with 1, 2, 3, or 4 independently selected R²⁰ groups. In some embodiments, R²⁸ is C₁₋₆ heteroalkyl substituted with 1, 2, 3, or 4 independently selected R²⁰ groups. In some embodiments, R²⁸ is C₂₋₆ alkenyl substituted with 1, 2, 3, or 4 independently selected R²⁰ groups. In some embodiments, R²⁸ is C₂₋₆ alkynyl substituted with 1, 2, 3, or 4 independently selected R²⁰ groups.

In some embodiments of a compound of Formula (I), (Ia), (IIa), (IIb), (IIc), or (IId), R²⁸ is selected from the group consisting of H, azido, halo, C₁₋₆ alkyl, C₂₋₆ alkenyl, C₂₋₆ alkynyl, C₁₋₆ heteroalkyl, —CN, —NO₂, —OR^{a7}, —C(═O)R^{b7}, —C(═O)OR^{b7}, —NR^{c7}R^{d7}, —C(═O)NR^{c7}R^{d7}, —OC(═O)NR^{c7}R^{d7}, —NR^{c7}C(═O)R^{b7}, —NR^{c7}C(═O)OR^{b7}, —NR^{c7}C(═O)NR^{c7}R^{d7}, —NR^{c7}S(═O)₂R^{b7}, and —NR^{c7}S(═O)₂NR^{c7}R^{d7}. In some embodiments, R²⁸ is H. In some embodiments, R²⁸ is azido. In some embodiments, R²⁸ is halo. In some embodiments, R²⁸ is C₁₋₆ alkyl. In some embodiments, R²⁸ is C₂₋₆ alkenyl. In some embodiments, R²⁸ is C₂₋₆ alkynyl. In some embodiments, R²⁸ is C₁₋₆ heteroalkyl. In some embodiments, R²⁸ is —CN. In some embodiments, R²⁸ is —NO₂. In some embodiments, R²⁸ is —OR^{a7}. In some embodiments, R²⁸ is —C(═O)R^{b7}. In some embodiments, R²⁸ is —C(═O)OR^{b7}. In some embodiments, R²⁸ is —NR^{c7}R^{d7}. In some embodiments, R²⁸ is —C(═O)NR^{c7}R^{d7}. In some embodiments, R²⁸ is —OC(═O)NR^{c7}R^{d7}. In some embodiments, R²⁸ is —NR^{c7}C(═O)R^{b7}. In some embodiments, R²⁸ is —NR^{c7}C(═O)OR^{b7}. In some embodiments, R²⁸ is —NR^{c7}C(═O)NR^{c7}R^{d7}. In some embodiments, R²⁸ is —NR^{c7}S(═O)₂R^{b7}. In some embodiments, R²⁸ is —NR^{c7}S(═O)₂NR^{c7}R^{d7}. In some embodiments of a compound of Formula (I), (Ia), (IIa), (IIb), (IIc), or (IId), X⁸ is CR²⁸ and R²⁸ is not H.

In some embodiments of a compound of Formula (I), (Ia), (IIa), (IIb), (IIc), or (IId), R²⁸ is F, Cl, —CN, —CH₃, —C≡CH,

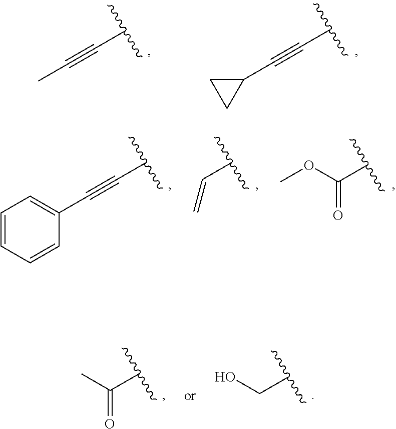

In some embodiments, R²⁸ is F. In some embodiments, R²⁸ is Cl. In some embodiments, R²⁸ is —CN. In some embodiments, R²⁸ is —CH₃. In some embodiments, R²⁸ is —C≡CH. In some embodiments, R²¹ is

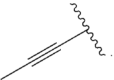 [SMILES: *C#CC]

In some embodiments, R²⁸ is

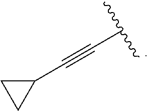 [SMILES: *C#CC1CC1]

In some embodiments, R²⁸ is

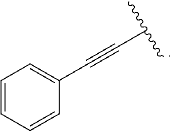 [SMILES: *C#Cc1ccccc1]

In some embodiments, R²⁸ is

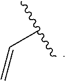 [SMILES: *C=C ; "."]

In some embodiments, R²⁸ is

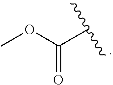 [SMILES: *C(=O)OC]

In some embodiments, R²⁸ is

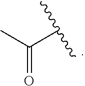 [SMILES: *C(C)=O]

In some embodiments, R²⁸ is

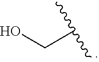 [SMILES: *CO]

In some embodiments, each R^{a3}, R^{b3}, R^{c3}, R^{d3}, R^{a4}, R^{b4}, R^{c4}, R^{d4} R^{a7}, R^{b7}, R^{c7}, R^{d7}, R^{a8}, R^{b8}, R^{c8}, and R^{d8} is independently selected from the group consisting of hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, and 4-10 membered heterocycloalkyl, wherein the C₁₋₆ alkyl, C₂₋₆ alkenyl, C₂₋₆ alkynyl, C₃₋₁₀ cycloalkyl, —(C₁₋₆ alkylene)-C₃₋₁₀ cycloalkyl, C₆₋₁₀ aryl, 5-10 membered heteroaryl, and 4-10 membered heterocycloalkyl are unsubstituted or substituted independently with 1, 2, 3, or 4 R²⁰ groups.

In some embodiments, R^{a3} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{a3} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{a3} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{a3} is C₆₋₁₀ aryl, 5-10 membered heteroaryl, or 4-10 membered heterocycloalkyl. In some embodiments, R^{a3} is hydrogen, C₁₋₆ alkyl, or C₁₋₆ haloalkyl. In some embodiments, R^{a3} is hydrogen. In some embodiments, R^{a3} is C₁₋₆ alkyl. In some embodiments, R^{a3} is methyl. In some embodiments, R^{a3} is ethyl. In some embodiments, R^{a3} is propyl. In some embodiments, R^{a3} is C₁₋₆ haloalkyl.

In some embodiments, R^{b3} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{b3} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{b3} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{b3} is C₆₋₁₀ aryl, 5-10 membered heteroaryl, or 4-10 membered heterocycloalkyl. In some embodiments, R^{b3} is hydrogen, C₁₋₆ alkyl, or C₁₋₆ haloalkyl. In some embodiments, R^{b3} is hydrogen. In some embodiments, R^{b3} is C₁₋₆ alkyl. In some embodiments, R^{b3} is methyl. In some embodiments, R^{b3} is ethyl. In some embodiments, R^{b3} is propyl. In some embodiments, R^{b3} is C₁₋₆ haloalkyl.

In some embodiments, R^{c3} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{c3} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R³ is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{c3} is C₆₋₁₀ aryl, 5-10 membered heteroaryl, or 4-10 membered heterocycloalkyl. In some embodiments, R^{c3} is hydrogen, C₁₋₆ alkyl, or C₁₋₆ haloalkyl. In some embodiments, R^{c3} is hydrogen. In some embodiments, R^{c3} is C₁₋₆ alkyl. In some embodiments, R^{c3} is methyl. In some embodiments, R^{c3} is ethyl. In some embodiments, R^{c3} is propyl. In some embodiments, R^{c3} is C₁₋₆ haloalkyl.

In some embodiments, R^{d3} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{d3} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{d3} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{d3} is C₆₋₁₀ aryl, 5-10 membered heteroaryl, or 4-10 membered heterocycloalkyl. In some embodiments, R^{d3} is hydrogen, C₁₋₆ alkyl, or C₁₋₆ haloalkyl. In some embodiments, R^{d3} is hydrogen. In some embodiments, R^{d3} is C₁₋₆ alkyl. In some embodiments, R^{d3} is methyl. In some embodiments, R^{d3} is ethyl. In some embodiments, R^{d3} is propyl. In some embodiments, R^{d3} is C₁₋₆ haloalkyl.

In some embodiments, R^{a4} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{a4} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{a4} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{a4} is C₆₋₁₀ aryl, 5-10 membered heteroaryl, or 4-10 membered heterocycloalkyl. In some embodiments, R^{a4} is hydrogen, C₁₋₆ alkyl, or C₁₋₆ haloalkyl. In some embodiments, R^{a4} is hydrogen. In some embodiments, R^{a4} is C₁₋₆ alkyl. In some embodiments, R^{a4} is methyl. In some embodiments, R^{a4} is ethyl. In some embodiments, R^{a4} is propyl. In some embodiments, R^{a4} is C₁₋₆ haloalkyl.

In some embodiments, R^{b4} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{b4} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{b4} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{b4} is C₆₋₁₀ aryl, 5-10 membered heteroaryl, or 4-10 membered heterocycloalkyl. In some embodiments, R^{b4} is hydrogen, C₁₋₆ alkyl, or C₁₋₆ haloalkyl. In some embodiments, R^{b4} is hydrogen. In some embodiments, R^{b4} is C₁₋₆ alkyl. In some embodiments, R^{b4} is methyl. In some embodiments, R^{b4} is ethyl. In some embodiments, R^{b4} is propyl. In some embodiments, R^{b4} is C₁₋₆ haloalkyl.

In some embodiments, R^{c4} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{c4} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R⁴ is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{c4} is C₆₋₁₀ aryl, 5-10 membered heteroaryl, or 4-10 membered heterocycloalkyl. In some embodiments, R^{c4} is hydrogen, C₁₋₆ alkyl, or C₁₋₆ haloalkyl. In some embodiments, R^{c4} is hydrogen. In some embodiments, R^{c4} is C₁₋₆ alkyl. In some embodiments, R^{c4} is methyl. In some embodiments, R^{c4} is ethyl. In some embodiments, R^{c4} is propyl. In some embodiments, R^{c4} is C₁₋₆ haloalkyl.

In some embodiments, R^{d4} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{d4} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{d4} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{d4} is C₆₋₁₀ aryl, 5-10 membered heteroaryl, or 4-10 membered heterocycloalkyl. In some embodiments, R^{d4} is hydrogen, C₁₋₆ alkyl, or C₁₋₆ haloalkyl. In some embodiments, R^{d4} is hydrogen. In some embodiments, R^{d4} is C₁₋₆ alkyl. In some embodiments, R^{d4} is methyl. In some embodiments, R^{d4} is ethyl. In some embodiments, R^{d4} is propyl. In some embodiments, R^{d4} is C₁₋₆ haloalkyl.

In some embodiments, R^{a7} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{a7} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{a7} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{a7} is C₆₋₁₀ aryl, 5-10 membered heteroaryl, or 4-10 membered heterocycloalkyl. In some embodiments, R^{a7} is hydrogen, C₁₋₆ alkyl, or C₁₋₆ haloalkyl. In some embodiments, R^{a7} is hydrogen. In some embodiments, R^{a7} is C₁₋₆ alkyl. In some embodiments, R^{a7} is methyl. In some embodiments, R^{a7} is ethyl. In some embodiments, R^{a7} is propyl. In some embodiments, R^{a7} is C₁₋₆ haloalkyl.

In some embodiments, R^{b7} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{b7} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{b7} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{b7} is C₆₋₁₀ aryl, 5-10 membered heteroaryl, or 4-10 membered heterocycloalkyl. In some embodiments, R^{b7} is hydrogen, C₁₋₆ alkyl, or C₁₋₆ haloalkyl. In some embodiments, R^{b7} is hydrogen. In some embodiments, R^{b7} is C₁₋₆ alkyl. In some embodiments, R^{b7} is methyl. In some embodiments, R^{b7} is ethyl. In some embodiments, R^{b7} is propyl. In some embodiments, R^{b7} is C₁₋₆ haloalkyl.

In some embodiments, R^{c7} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{c7} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{c7} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{c7} is C₆₋₁₀ aryl, 5-10 membered heteroaryl, or 4-10 membered heterocycloalkyl. In some embodiments, R^{c7} is hydrogen, C₁₋₆ alkyl, or C₁₋₆ haloalkyl. In some embodiments, R^{c7} is hydrogen. In some embodiments, R^{c7} is C₁₋₆ alkyl. In some embodiments, R^{c7} is methyl. In some embodiments, R^{c7} is ethyl. In some embodiments, R^{c7} is propyl. In some embodiments, R^{c7} is C₁₋₆ haloalkyl.

In some embodiments, R^{d7} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{d7} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{d7} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{d7} is C₆₋₁₀ aryl, 5-10 membered heteroaryl, or 4-10 membered heterocycloalkyl. In some embodiments, R^{d7} is hydrogen, C₁₋₆ alkyl, or C₁₋₆ haloalkyl. In some embodiments, R^{d7} is hydrogen. In some embodiments, R^{d7} is C₁₋₆ alkyl. In some embodiments, R^{d7} is methyl. In some embodiments, R^{d7} is ethyl. In some embodiments, R^{d7} is propyl. In some embodiments, R^{d7} is C₁₋₆ haloalkyl.

In some embodiments, R^{a8} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{a8} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{a8} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{a8} is C₆₋₁₀ aryl, 5-10 membered heteroaryl, or 4-10 membered heterocycloalkyl. In some embodiments, R^{a8} is hydrogen, C₁₋₆ alkyl, or C₁₋₆ haloalkyl. In some embodiments, R^{a8} is hydrogen. In some embodiments, R^{a8} is C₁₋₆ alkyl. In some embodiments, R^{a8} is methyl. In some embodiments, R^{a8} is ethyl. In some embodiments, R^{a8} is propyl. In some embodiments, R^{a8} is C₁₋₆ haloalkyl.

In some embodiments, R^{b8} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{b8} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{b8} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{b8} is C₆₋₁₀ aryl, 5-10 membered heteroaryl, or 4-10 membered heterocycloalkyl. In some embodiments, R^{b8} is hydrogen, C₁₋₆ alkyl, or C₁₋₆ haloalkyl. In some embodiments, R^{b8} is hydrogen. In some embodiments, R^{b8} is C₁₋₆ alkyl. In some embodiments, R^{b8} is methyl. In some embodiments, R^{b8} is ethyl. In some embodiments, R^{b8} is propyl. In some embodiments, R^{b8} is C₁₋₆ haloalkyl.

In some embodiments, R^{c8} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{c8} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{c8} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{c8} is C₆₋₁₀ aryl, 5-10 membered heteroaryl, or 4-10 membered heterocycloalkyl. In some embodiments, R^{c8} is hydrogen, C₁₋₆ alkyl, or C₁₋₆ haloalkyl. In some embodiments, R^{c8} is hydrogen. In some embodiments, R^{c8} is C₁₋₆ alkyl. In some embodiments, R^{c8} is methyl. In some embodiments, R^{c8} is ethyl. In some embodiments, R^{c8} is propyl. In some embodiments, R^{c8} is C₁₋₆ haloalkyl.

In some embodiments, R^{d8} is hydrogen, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, or 4-10 membered heterocycloalkyl. In some embodiments, R^{d8} is hydrogen, C₁₋₆ alkyl, C₂₋₆ alkenyl, C₂₋₆ alkynyl, C₁₋₆ hydroxyalkyl, C₁₋₆ haloalkyl, or C₁₋₆ alkoxy. In some embodiments, R^{d8} is —(C₁₋₆ alkylene)-C₁₋₆ alkoxy, C₃₋₁₀ cycloalkyl, or —(C₁₋₆ alkylene)-C₃₋₁₀ cycloalkyl. In some embodiments, R^{d8} is C₆₋₁₀ aryl, 5-10 membered heteroaryl, or 4-10 membered heterocycloalkyl. In some embodiments, R^{d8} is hydrogen, C₁₋₆ alkyl, or C₁₋₆ haloalkyl. In some embodiments, R^{d8} is hydrogen. In some embodiments, R^{d8} is C₁₋₆ alkyl. In some embodiments, R^{d8} is methyl. In some embodiments, R^{d8} is ethyl. In some embodiments, R^{d8} is propyl. In some embodiments, R^{d8} is C₁₋₆ haloalkyl.

In some embodiments, R^{c3} and R^{d3} together with the N atom to which they are connected, come together to form a 5-10 membered heteroaryl or 4-10 membered heterocycloalkyl, wherein the 5-10 membered heteroaryl and 4-10 membered heterocycloalkyl are unsubstituted or substituted independently with 1, 2, 3, or 4 R²⁰ groups.

In some embodiments, R^{c3} and R^{d3} together with the N atom to which they are connected, come together to form a 5-10 membered heteroaryl or 4-10 membered heterocycloalkyl.

In some embodiments, R^{c4} and R^{d4} together with the N atom to which they are connected, come together to form a 5-10 membered heteroaryl or 4-10 membered heterocycloalkyl, wherein the 5-10 membered heteroaryl and 4-10 membered heterocycloalkyl are unsubstituted or substituted independently with 1, 2, 3, or 4 R²⁰ groups.

In some embodiments, R^{c4} and R^{d4} together with the N atom to which they are connected, come together to form a 5-10 membered heteroaryl or 4-10 membered heterocycloalkyl.

In some embodiments, R^{c7} and R^{d7} together with the N atom to which they are connected, come together to form a 5-10 membered heteroaryl or 4-10 membered heterocycloalkyl, wherein the 5-10 membered heteroaryl and 4-10 membered heterocycloalkyl are unsubstituted or substituted independently with 1, 2, 3, or 4 R²⁰ groups.

In some embodiments, R^{c7} and R^{d7} together with the N atom to which they are connected, come together to form a 5-10 membered heteroaryl or 4-10 membered heterocycloalkyl.

In some embodiments, R^{c8} and R^{d8} together with the N atom to which they are connected, come together to form a 5-10 membered heteroaryl or 4-10 membered heterocycloalkyl, wherein the 5-10 membered heteroaryl and 4-10 membered heterocycloalkyl are unsubstituted or substituted independently with 1, 2, 3, or 4 R²⁰ groups.

In some embodiments, R^{c8} and R^{d8} together with the N atom to which they are connected, come together to form a 5-10 membered heteroaryl or 4-10 membered heterocycloalkyl.

In some embodiments, the compound is selected from Table 1.

In some embodiments, a SMSM described herein, possesses one or more stereocenters and each stereocenter exists independently in either the R or S configuration.

The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some cases, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments of a compound disclosed herein, one or more of R²⁰, R^{20a}, R^{20b}, R^{20c}, R^{20d}, R²¹, R²³, R²⁴, R²⁷, R²⁸, R³¹, R³², R^{a3}, R^{b3}, R^{c3}, R^{d3}, R^{a4}, R^{b4}, R^{c4}, R^{d4}, R^{a7}, R^{b7}, R^{c7}, R^{d7}, R^{a8}, R^{b8}, R^{c8}, and R^{d8} groups comprise deuterium at a percentage higher than the natural abundance of deuterium.

In some embodiments of a compound disclosed herein, one or more ¹H are replaced with one or more deuteriums in one or more of the following groups R²⁰, R^{20a}, R^{20b}, R^{20c}, R^{20d}, R²¹, R²³, R²⁴, R²⁷, R²⁸, R³¹, R³², R^{a3}, R^{b3}, R^{c3}, R^{d3}, R^{a4}, R^{b4}, R^{c4}, R^{d4}, R^{a7}, R^{b7}, R^{c7}, R^{d7}, R^{a8}, R^{b8}, R^{c8}, and R^{d8}.

In some embodiments of a compound disclosed herein, the abundance of deuterium in each of R²⁰, R^{20a}, R^{20b}, R^{2c}, R^{20d}, R²¹, R²³, R²⁴, R²⁷, R²⁸, R³¹, R³², R^{a3}, R^{b3}, R^{c3}, R^{d3}, R^{a4}, R^{b4}, R^{c4}, R^{d4}, R^{a7}, R^{b7}, R^{c7}, R^{d7}, R^{a8}, R^{b8}, R^{c8}, and R^{d8} is independently at least 1%, at least 10%, at least 20%, at least 30%, at least 40%, at least 50%, at least 60%, at least 70%, at least 80%, at least 90%, or 100% by molar.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent and may be formed during the process of crystallization with pharmaceutically acceptable solvents such as water, ethanol, and the like. Hydrates are formed when the solvent is water, or alcoholates are formed when the solvent is alcohol. In some embodiments, solvates of compounds described herein are conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In some embodiments, a SMSM has a molecular weight of at most about 2000 Daltons, 1500 Daltons, 1000 Daltons or 900 Daltons. In some embodiments, a SMSM has a molecular weight of at least 100 Daltons, 200 Daltons, 300 Daltons, 400 Daltons or 500 Daltons. In some embodiments, a SMSM does not comprise a phosphodiester linkage. In some embodiments, a SMSM is a compound with a structure set forth in Table 1 below.

TABLE 1
Exemplary SMSM compounds

| Compound # | Structure | IUPAC Name |
|---|---|---|
| 1 | 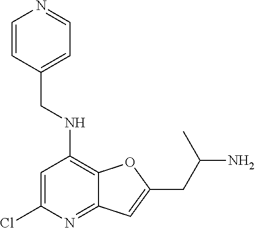 [SMILES: CC(N)Cc1cc2nc(Cl)cc(NCc3ccncc3)c2o1] | 2-(2-aminopropyl)-5-chloro-7-{[(4- pyridyl)methyl]amino}-1-oxa-4-azaindene |
| 2 | 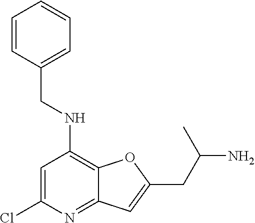 [SMILES: CC(N)Cc1cc2nc(Cl)cc(NCc3ccccc3)c2o1] | 2-(2-aminopropyl)-7-benzylamino-5- chloro-1-oxa-4-azaindene |
| 3 | 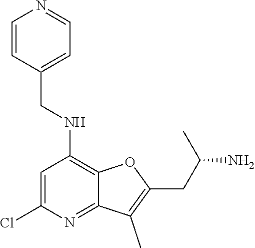 [SMILES: Cc1c(C[C@H](C)N)oc2c(NCc3ccncc3)cc(Cl)nc12] | 2-[(S)-2-aminopropyl]-5-chloro-3-methyl- 7-{[(4-pyridyl)methyl]amino}-1-oxa-4- azaindene |
| and | | |
| 4 | 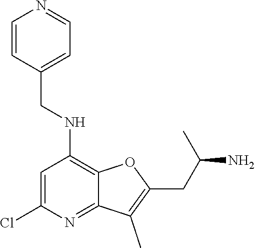 [SMILES: Cc1c(C[C@@H](C)N)oc2c(NCc3ccncc3)cc(Cl)nc12] | 2-[(R)-2-aminopropyl]-5-chloro-3-methyl- 7-{[(4-pyridyl)methyl]amino}-1-oxa-4- azaindene |
| 5 | 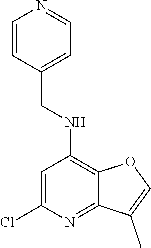 [SMILES: Cc1coc2c(NCc3ccncc3)cc(Cl)nc12] | [(4-pyridyl)methyl](5-chloro-3-methyl-1- oxa-4-aza-7-indenyl)amine |
| 6 | 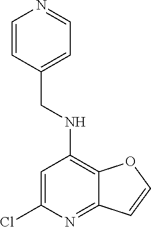 [SMILES: Clc1cc(NCc2ccncc2)c2occc2n1] | [(4-pyridyl) methyl](5-chloro-1-oxa-4-aza- 7-indenyl)amine |
| 7 | 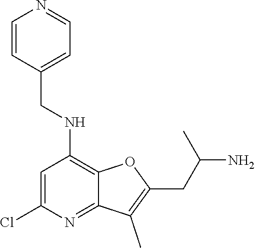 [SMILES: Cc1c(CC(C)N)oc2c(NCc3ccncc3)cc(Cl)nc12] | 2-(2-aminopropyl)-5-chloro-3-methyl-7- {[(4-pyridyl)methyl]amino}-1-oxa-4- azaindene |
| 8 | 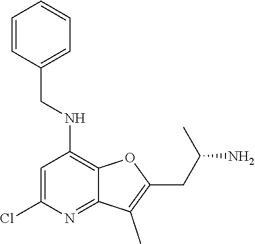 [SMILES: Cc1c(C[C@H](C)N)oc2c(NCc3ccccc3)cc(Cl)nc12] | 2-[(S)-2-aminopropyl]-7-benzylamino-5- chloro-3-methyl-1-oxa-4-azaindene |
| 9 | 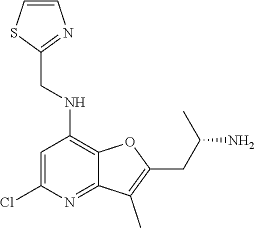 [SMILES: Cc1c(C[C@H](C)N)oc2c(NCc3nccs3)cc(Cl)nc12] | 2-[(S)-2-aminopropyl]-5-chloro-3-methyl- 7-{[(1,3-thiazol-2-yl)methyl]amino}-1-oxa- 4-azaindene |
| 10 | 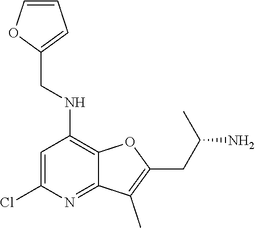 [SMILES: Cc1c(C[C@H](C)N)oc2c(NCc3ccco3)cc(Cl)nc12] | 2-[(S)-2-aminopropyl]-5-chloro-7- (furfurylamino)-3-methyl-1-oxa-4- azaindene |
| 11 | 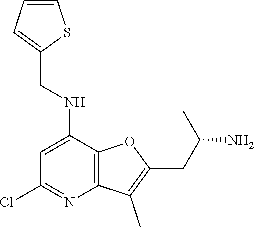 [SMILES: Cc1c(C[C@H](C)N)oc2c(NCc3cccs3)cc(Cl)nc12] | 2-[(S)-2-aminopropyl]-5-chloro-3-methyl- 7-thenylamino-1-oxa-4-azaindene |
| 12 | 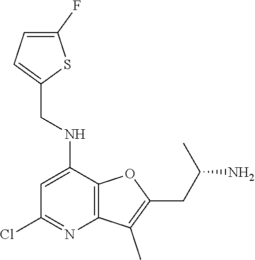 [SMILES: Cc1c(C[C@H](C)N)oc2c(NCc3ccc(F)s3)cc(Cl)nc12] | 2-[(S)-2-aminopropyl]-5-chloro-7-{[(5- fluoro-2-thienyl)methyl]amino}-3-methyl- 1-oxa-4-azaindene |
| 13 | 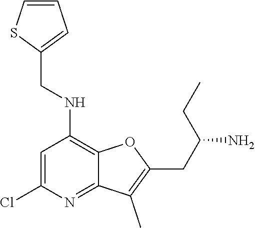 [SMILES: CC[C@H](N)Cc1oc2c(NCc3cccs3)cc(Cl)nc2c1C] | 2-[(S)-2-aminobutyl]-5-chloro-3-methyl-7- thenylamino-1-oxa-4-azaindene |
| 14 | 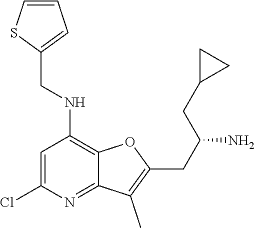 [SMILES: Cc1c(C[C@@H](N)CC2CC2)oc2c(NCc3cccs3)cc(Cl)nc12] | 2-[(S)-2-amino-3-cyclopropylpropyl]-5- chloro-3-methyl-7-thenylamino-1-oxa-4- azaindene |
| 15 | 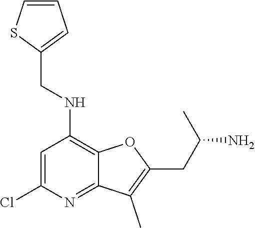 [SMILES: Cc1c(C[C@H](C)N)oc2c(NCc3cccs3)cc(Cl)nc12] | 2-[(S)-2-aminopropyl]-3-methyl-7- thenylamino-1-oxa-4-aza-5- indenecarbonitrile |
| 16 | 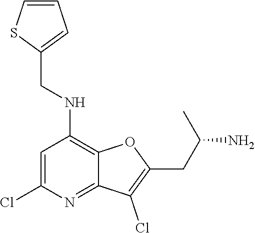 [SMILES: C[C@H](N)Cc1oc2c(NCc3cccs3)cc(Cl)nc2c1Cl] | 2-[(S)-2-aminopropyl]-3,5-dichloro-7- thenylamino-1-oxa-4-azaindene |
| 17 | 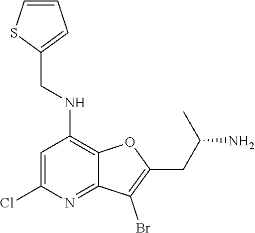 [SMILES: C[C@H](N)Cc1oc2c(NCc3cccs3)cc(Cl)nc2c1Br] | 2-[(S)-2-aminopropyl]-3-bromo-5-chloro- 7-thenylamino-1-oxa-4-azaindene |
| 18 | 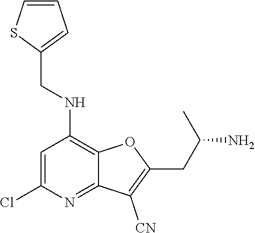 [SMILES: C[C@H](N)Cc1oc2c(NCc3cccs3)cc(Cl)nc2c1C#N] | 2-[(S)-2-aminopropyl]-5-chloro-7- thenylamino-1-oxa-4-aza-3- indenecarbonitrile |
| 19 | 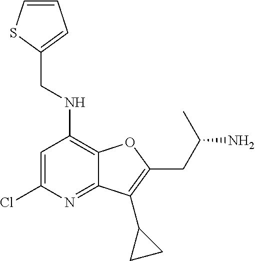 [SMILES: C[C@H](N)Cc1oc2c(NCc3cccs3)cc(Cl)nc2c1C1CC1] | 2-[(S)-2-aminopropyl]-5-chloro-3- cyclopropyl-7-thenylamino-1-oxa-4- azaindene |
| 20 | 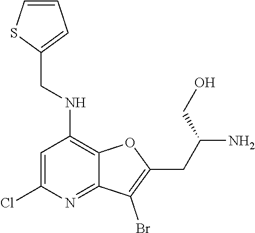 [SMILES: N[C@@H](CO)Cc1oc2c(NCc3cccs3)cc(Cl)nc2c1Br] | (R)-2-amino-3-(3-bromo-5-chloro-7- thenylamino-1-oxa-4-aza-2-indenyl)-1- propanol |
| 21 | 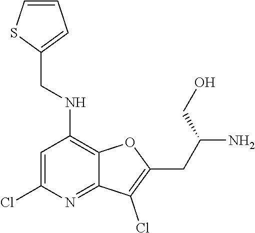 [SMILES: N[C@@H](CO)Cc1oc2c(NCc3cccs3)cc(Cl)nc2c1Cl] | (R)-2-amino-3-(3,5-dichloro-7- thenylamino-1-oxa-4-aza-2-indenyl)-1- propanol |
| 22 | 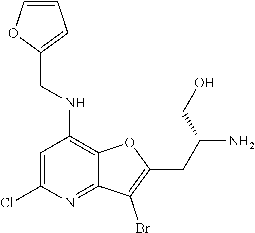 [SMILES: N[C@@H](CO)Cc1oc2c(NCc3ccco3)cc(Cl)nc2c1Br] | (R)-2-amino-3-[3-bromo-5-chloro-7- (furfurylamino)-1-oxa-4-aza-2-indenyl]-1- propanol |
| 23 | 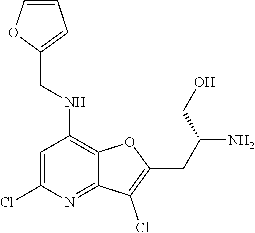 [SMILES: N[C@@H](CO)Cc1oc2c(NCc3ccco3)cc(Cl)nc2c1Cl] | (R)-2-amino-3-[3,5-dichloro-7- (furfurylamino)-1-oxa-4-aza-2-indenyl]-1- propanol |
| 24 | 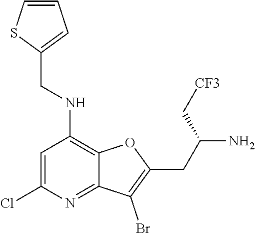 [SMILES: N[C@H](Cc1oc2c(NCc3cccs3)cc(Cl)nc2c1Br)CC(F)(F)F] | 2-[(R)-2-amino-4,4,4-trifluorobutyl]-3- bromo-5-chloro-7-thenylamino-1-oxa-4- azaindene |
| 25 | 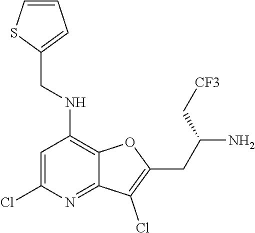 [SMILES: N[C@H](Cc1oc2c(NCc3cccs3)cc(Cl)nc2c1Cl)CC(F)(F)F] | 2-[(R)-2-amino-4,4,4-trifluorobutyl]-3,5- dichloro-7-thenylamino-1-oxa-4- azaindene |
| 26 | 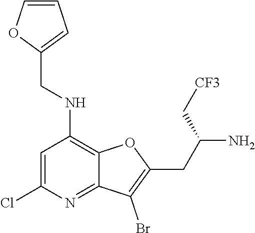 [SMILES: N[C@H](Cc1oc2c(NCc3ccco3)cc(Cl)nc2c1Br)CC(F)(F)F] | 2-[(R)-2-amino-4,4,4-trifluorobutyl]-3- bromo-5-chloro-7-(furfurylamino)-1-oxa- 4-azaindene |
| 27 | 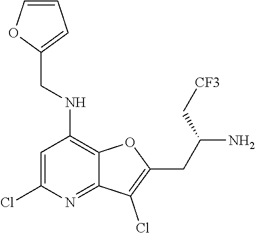 [SMILES: N[C@H](Cc1oc2c(NCc3ccco3)cc(Cl)nc2c1Cl)CC(F)(F)F] | 2-[(R)-2-amino-4,4,4-trifluorobutyl]-3,5- dichloro-7-(furfurylamino)-1-oxa-4- azaindene |
| 28 | 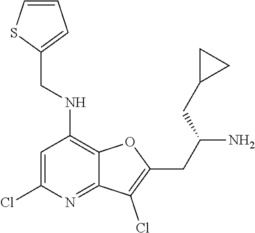 [SMILES: N[C@H](Cc1oc2c(NCc3cccs3)cc(Cl)nc2c1Cl)CC1CC1] | 2-[(S)-2-amino-3-cyclopropylpropyl]-3,5- dichloro-7-thenylamino-1-oxa-4- azaindene |
| 29 | 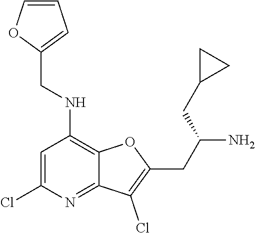 [SMILES: N[C@H](Cc1oc2c(NCc3ccco3)cc(Cl)nc2c1Cl)CC1CC1] | 2-[(S)-2-amino-3-cyclopropylpropyl]-3,5- dichloro-7-(furfurylamino)-1-oxa-4- azaindene |
| 30 | 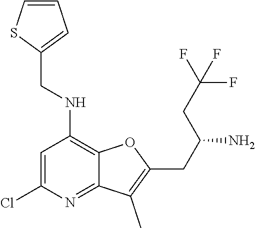 [SMILES: Cc1c(C[C@@H](N)CC(F)(F)F)oc2c(NCc3cccs3)cc(Cl)nc12] | 2-[(R)-2-amino-4,4,4-trifluorobutyl]-5- chloro-3-methyl-7-thenylamino-1-oxa-4- azaindene |
| 31 | 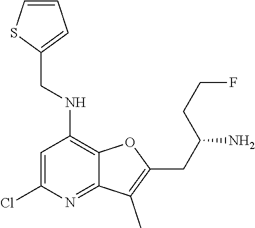 [SMILES: Cc1c(C[C@@H](N)CCF)oc2c(NCc3cccs3)cc(Cl)nc12] | 2-[(R)-2-amino-4-fluorobutyl]-5-chloro-3- methyl-7-thenylamino-1-oxa-4-azaindene |
| 32 | 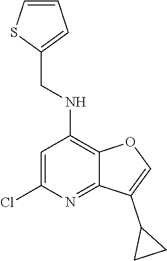 [SMILES: Clc1cc(NCc2cccs2)c2occ(C3CC3)c2n1] | (thenyl)(5-chloro-3-cyclopropyl-1-oxa-4- aza-7-indenyl)amine |
| 33 | 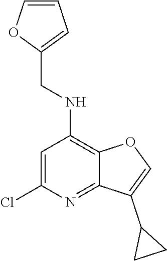 [SMILES: Clc1cc(NCc2ccco2)c2occ(C3CC3)c2n1] | (furfuryl)(5-chloro-3-cyclopropyl-1-oxa-4- aza-7-indenyl)amine |
| 34 | 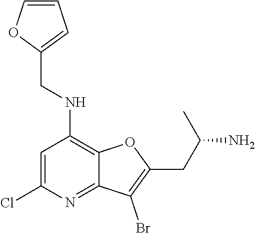 [SMILES: C[C@H](N)Cc1oc2c(NCc3ccco3)cc(Cl)nc2c1Br] | 2-[(S)-2-aminopropyl]-3-bromo-5-chloro- 7-(furfurylamino)-1-oxa-4-azaindene |
| 35 | 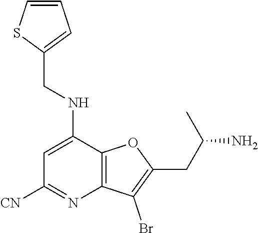 | 2-[(S)-2-aminopropyl]-3-bromo-7- thenylamino-1-oxa-4-aza-5- indenecarbonitrile |
| 36 | 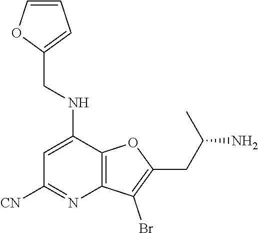 | 2-[(S)-2-aminopropyl]-3-bromo-7- (furfurylamino)-1-oxa-4-aza-5- indenecarbonitrile |
| 37 | 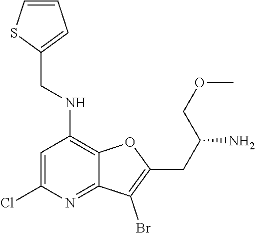 [SMILES: COC[C@H](N)Cc1oc2c(NCc3cccs3)cc(Cl)nc2c1Br] | 2-[(R)-2-amino-3-methoxypropyl]-3- bromo-5-chloro-7-thenylamino-1-oxa-4- azaindene |
| 38 | 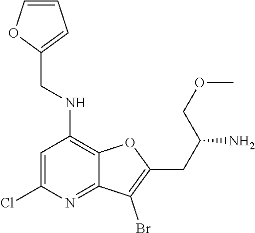 [SMILES: COC[C@H](N)Cc1oc2c(NCc3ccco3)cc(Cl)nc2c1Br] | 2-[(R)-2-amino-3-methoxypropyl]-3- bromo-5-chloro-7-(furfurylamino)-1-oxa- 4-azaindene |
| 39 | 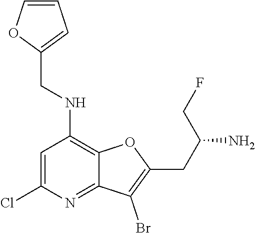 [SMILES: N[C@@H](CF)Cc1oc2c(NCc3ccco3)cc(Cl)nc2c1Br] | 2-[(R)-2-amino-3-fluoropropyl]-3-bromo- 5-chloro-7-(furfurylamino)-1-oxa-4- azaindene |
| 40 | 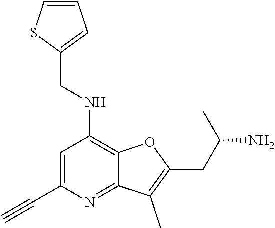 [SMILES: C#Cc1cc(NCc2cccs2)c2oc(C[C@H](C)N)c(C)c2n1] | 2-[(S)-2-aminopropyl]-5-ethynyl-3- methyl-7-thenylamino-1-oxa-4-azaindene |
| 41 | 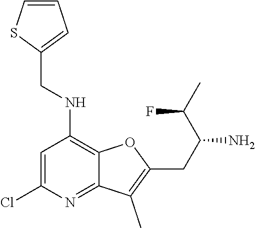 [SMILES: Cc1c(C[C@@H](N)[C@H](C)F)oc2c(NCc3cccs3)cc(Cl)nc12] | 2-[(2R,3S)-2-amino-3-fluorobutyl]-5- chloro-3-methyl-7-thenylamino-1-oxa-4- azaindene |
| 42 | 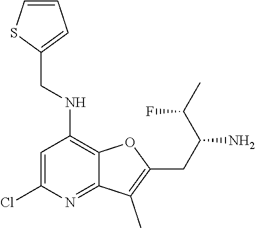 [SMILES: Cc1c(C[C@@H](N)[C@@H](C)F)oc2c(NCc3cccs3)cc(Cl)nc12] | 2-[(2R,3R)-2-amino-3-fluorobutyl]-5- chloro-3-methyl-7-thenylamino-1-oxa-4- azaindene |
| 43 | 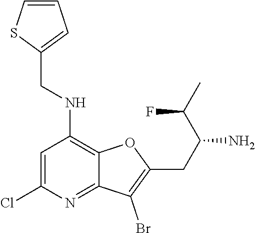 [SMILES: C[C@H](F)[C@H](N)Cc1oc2c(NCc3cccs3)cc(Cl)nc2c1Br] | 2-[(2R,3S)-2-amino-3-fluorobutyl]-3- bromo-5-chloro-7-thenylamino-1-oxa-4- azaindene |
| 44 | 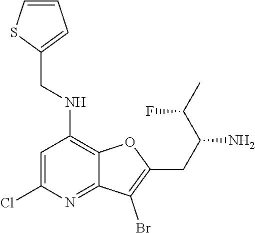 [SMILES: C[C@@H](F)[C@H](N)Cc1oc2c(NCc3cccs3)cc(Cl)nc2c1Br] | 2-[(2R,3R)-2-amino-3-fluorobutyl]-3- bromo-5-chloro-7-thenylamino-1-oxa-4- azaindene |
| 45 | 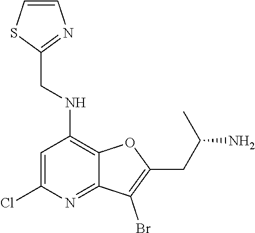 [SMILES: C[C@H](N)Cc1oc2c(NCc3nccs3)cc(Cl)nc2c1Br] | 2-[(S)-2-aminopropyl]-3-bromo-5-chloro- 7-{[(1,3-thiazol-2-yl)methyl]amino}-1-oxa- 4-azaindene |
| 46 | 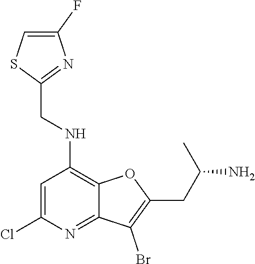 [SMILES: C[C@H](N)Cc1oc2c(NCc3nc(F)cs3)cc(Cl)nc2c1Br] | 2-[(S)-2-aminopropyl]-3-bromo-5-chloro- 7-{[(4-fluoro-1,3-thiazol-2- yl)methyl]amino}-1-oxa-4-azaindene |
| 47 | 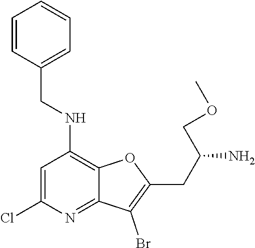 [SMILES: COC[C@H](N)Cc1oc2c(NCc3ccccc3)cc(Cl)nc2c1Br] | 2-[(R)-2-amino-3-methoxypropyl]-7- benzylamino-3-bromo-5-chloro-1-oxa-4- azaindene |
| 48 | 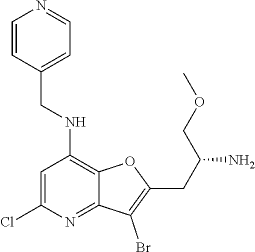 [SMILES: COC[C@H](N)Cc1oc2c(NCc3ccncc3)cc(Cl)nc2c1Br] | (R)-2-(2-amino-3-methoxypropyl)-3- bromo-5-chloro-N-(pyridin-4- ylmethyl)furo[3,2-b]pyridin-7-amine |
| 49 | 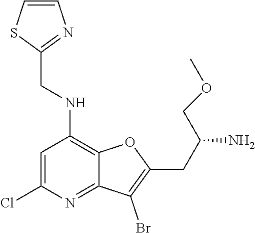 [SMILES: COC[C@H](N)Cc1oc2c(NCc3nccs3)cc(Cl)nc2c1Br] | 2-[(R)-2-amino-3-methoxypropyl]-3- bromo-5-chloro-7-{[(1,3-thiazol-2- yl)methyl]amino}-1-oxa-4-azaindene |
| 50 | 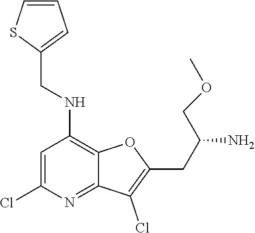 [SMILES: COC[C@H](N)Cc1oc2c(NCc3cccs3)cc(Cl)nc2c1Cl] | 2-[(R)-2-amino-3-methoxypropyl]-3,5- dichloro-7-thenylamino-1-oxa-4- azaindene |
| 51 | 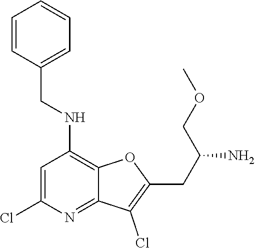 [SMILES: COC[C@H](N)Cc1oc2c(NCc3ccccc3)cc(Cl)nc2c1Cl] | 2-[(R)-2-amino-3-methoxypropyl]-7- benzylamino-3,5-dichloro-1-oxa-4- azaindene |
| 52 | 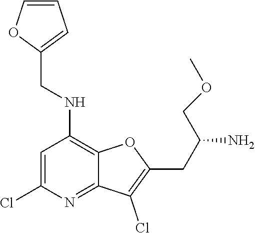 [SMILES: COC[C@H](N)Cc1oc2c(NCc3ccco3)cc(Cl)nc2c1Cl] | 2-[(R)-2-amino-3-methoxypropyl]-3,5- dichloro-7-(furfurylamino)-1-oxa-4- azaindene |
| 53 | 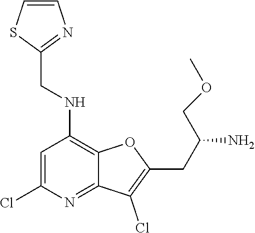 [SMILES: COC[C@H](N)Cc1oc2c(NCc3nccs3)cc(Cl)nc2c1Cl] | 2-[(R)-2-amino-3-methoxypropyl]-3,5- dichloro-7-{[(1,3-thiazol-2- yl)methyl]amino}-1-oxa-4-azaindene |
| 54 | 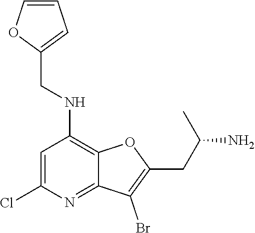 [SMILES: C[C@H](N)Cc1oc2c(NCc3ccco3)cc(Cl)nc2c1Br] | 2-[(S)-2-aminopropyl]-3,5-dichloro-7- (furfurylamino)-1-oxa-4-azaindene |
| 55 | 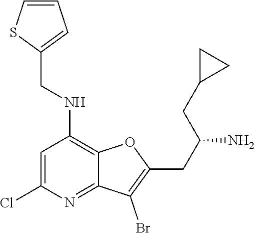 [SMILES: N[C@H](Cc1oc2c(NCc3cccs3)cc(Cl)nc2c1Br)CC1CC1] | 2-[(S)-2-amino-3-cyclopropylpropyl]-3- bromo-5-chloro-7-thenylamino-1-oxa-4- azaindene |
| 56 | 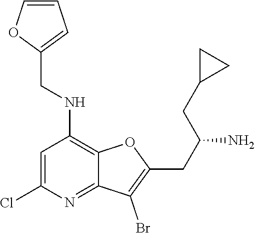 [SMILES: N[C@H](Cc1oc2c(NCc3ccco3)cc(Cl)nc2c1Br)CC1CC1] | 2-[(S)-2-amino-3-cyclopropylpropyl]-3- bromo-5-chloro-7-(furfurylamino)-1-oxa- 4-azaindene |
| 57 | 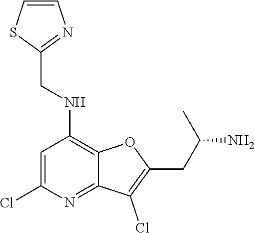 [SMILES: C[C@H](N)Cc1oc2c(NCc3nccs3)cc(Cl)nc2c1Cl] | 2-[(S)-2-aminopropyl]-3,5-dichloro-7- {[(1,3-thiazol-2-yl)methyl]amino}-1-oxa-4- azaindene |
| 58 | 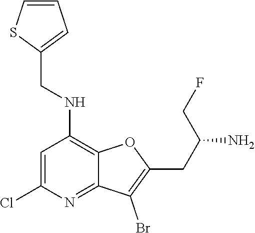 [SMILES: N[C@@H](CF)Cc1oc2c(NCc3cccs3)cc(Cl)nc2c1Br] | 2-[(R)-2-amino-3-fluoropropyl]-3-bromo- 5-chloro-7-thenylamino-1-oxa-4- azaindene |
| 59 | 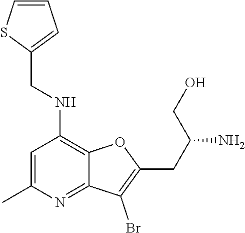 [SMILES: Cc1cc(NCc2cccs2)c2oc(C[C@@H](N)CO)c(Br)c2n1] | (R)-2-amino-3-(3-bromo-5-methyl-7- thenylamino-1-oxa-4-aza-2-indenyl)-1- propanol |
| 60 | 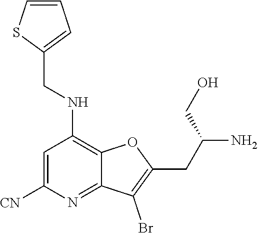 | 2-[(R)-2-amino-3-hydroxypropyl]-3- bromo-7-thenylamino-1-oxa-4-aza-5- indenecarbonitrile |
| 61 | 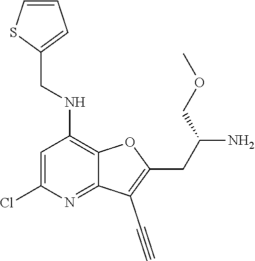 [SMILES: C#Cc1c(C[C@@H](N)COC)oc2c(NCc3cccs3)cc(Cl)nc12] | 2-[(R)-2-amino-3-methoxypropyl]-5- chloro-3-ethynyl-7-thenylamino-1-oxa-4- azaindene |
| 62 | 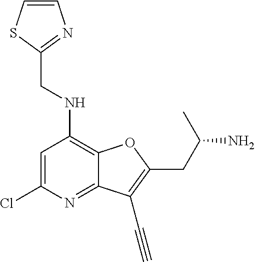 [SMILES: C#Cc1c(C[C@H](C)N)oc2c(NCc3nccs3)cc(Cl)nc12] | 2-[(S)-2-aminopropyl]-5-chloro-3-ethynyl- 7-{[(1,3-thiazol-2-yl)methyl]amino}-1-oxa- 4-azaindene |
| 63 | 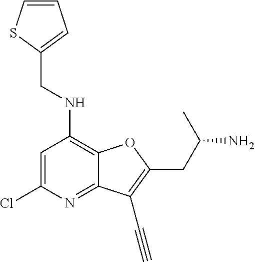 [SMILES: C#Cc1c(C[C@H](C)N)oc2c(NCc3cccs3)cc(Cl)nc12] | 2-[(S)-2-aminopropyl]-5-chloro-3-ethynyl- 7-thenylamino-1-oxa-4-azaindene |
| 64 | 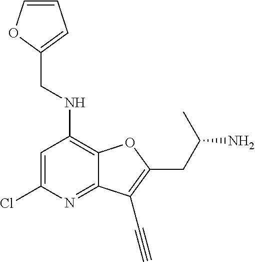 [SMILES: C#Cc1c(C[C@H](C)N)oc2c(NCc3ccco3)cc(Cl)nc12] | 2-[(S)-2-aminopropyl]-5-chloro-3-ethynyl- 7-(furfurylamino)-1-oxa-4-azaindene |
| 65 | 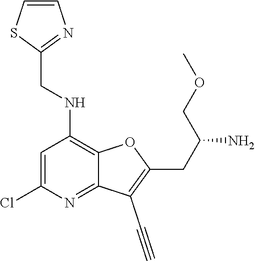 [SMILES: C#Cc1c(C[C@@H](N)COC)oc2c(NCc3nccs3)cc(Cl)nc12] | 2-[(R)-2-amino-3-methoxypropyl]-5- chloro-3-ethynyl-7-{[(1,3-thiazol-2- yl)methyl]amino}-1-oxa-4-azaindene |
| 66 | 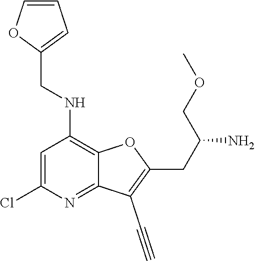 [SMILES: C#Cc1c(C[C@@H](N)COC)oc2c(NCc3ccco3)cc(Cl)nc12] | 2-[(R)-2-amino-3-methoxypropyl]-5- chloro-3-ethynyl-7-(furfurylamino)-1-oxa- 4-azaindene |
| 67 | 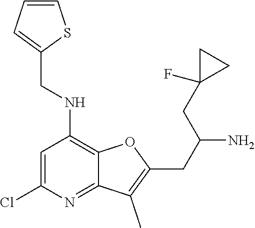 [SMILES: Cc1c(CC(N)CC2(F)CC2)oc2c(NCc3cccs3)cc(Cl)nc12] | 2-[2-amino-3-(1- fluorocyclopropyl)propyl]-5-chloro-3- methyl-7-thenylamino-1-oxa-4-azaindene |
| 68 | 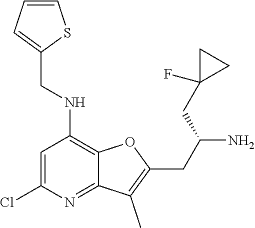 [SMILES: Cc1c(C[C@@H](N)CC2(F)CC2)oc2c(NCc3cccs3)cc(Cl)nc12] | 2-[(R)-2-amino-3-(1- fluorocyclopropyl)propyl]-5-chloro-3- methyl-7-thenylamino-1-oxa-4-azaindene |
| 69 | 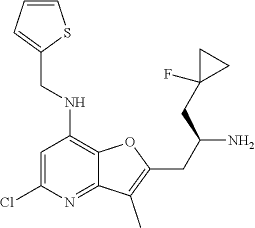 [SMILES: Cc1c(C[C@H](N)CC2(F)CC2)oc2c(NCc3cccs3)cc(Cl)nc12] | 2-[(S)-2-amino-3-(1- fluorocyclopropyl)propyl]-5-chloro-3- methyl-7-thenylamino-1-oxa-4-azaindene |
| 70 | 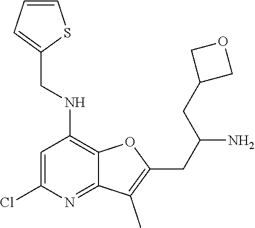 [SMILES: Cc1c(CC(N)CC2COC2)oc2c(NCc3cccs3)cc(Cl)nc12] | 2-[2-amino-3-(3-oxetanyl)propyl]-5- chloro-3-methyl-7-thenylamino-1-oxa-4- azaindene |
| 73 | 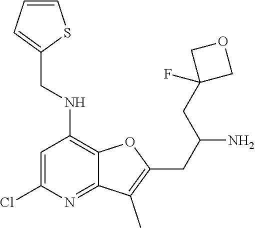 [SMILES: Cc1c(CC(N)CC2(F)COC2)oc2c(NCc3cccs3)cc(Cl)nc12] | 2-[2-amino-3-(3-fluoro-3- oxetanyl)propyl]-5-chloro-3-methyl-7- thenylamino-1-oxa-4-azaindene |
| 74 | 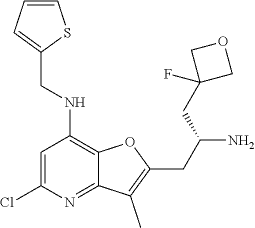 [SMILES: Cc1c(C[C@@H](N)CC2(F)COC2)oc2c(NCc3cccs3)cc(Cl)nc12] | 2-[(R)-2-amino-3-(3-fluoro-3- oxetanyl)propyl]-5-chloro-3-methyl-7- thenylamino-1-oxa-4-azaindene |
| 75 | 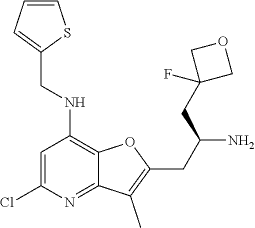 [SMILES: Cc1c(C[C@H](N)CC2(F)COC2)oc2c(NCc3cccs3)cc(Cl)nc12] | 2-[(S)-2-amino-3-(3-fluoro-3- oxetanyl)propyl]-5-chloro-3-methyl-7- thenylamino-1-oxa-4-azaindene |
| 76 | 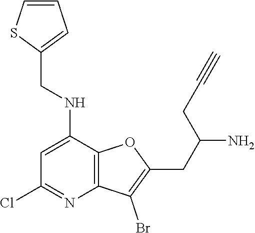 [SMILES: C#CCC(N)Cc1oc2c(NCc3cccs3)cc(Cl)nc2c1Br] | 2-(2-amino-4-pentynyl)-3-bromo-5- chloro-7-thenylamino-1-oxa-4-azaindene |
| 77 | 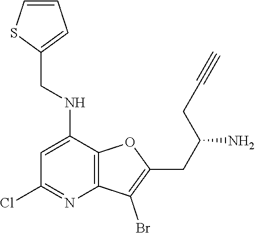 [SMILES: C#CC[C@H](N)Cc1oc2c(NCc3cccs3)cc(Cl)nc2c1Br] | 2-[(S)-2-amino-4-pentynyl]-3-bromo-5- chloro-7-thenylamino-1-oxa-4-azaindene |
| 78 | 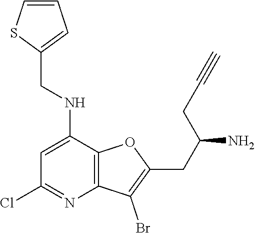 [SMILES: C#CC[C@@H](N)Cc1oc2c(NCc3cccs3)cc(Cl)nc2c1Br] | 2-[(R)-2-amino-4-pentynyl]-3-bromo-5- chloro-7-thenylamino-1-oxa-4-azaindene |
| 79 | 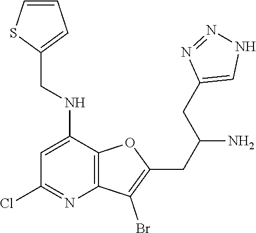 [SMILES: NC(Cc1c[nH]nn1)Cc1oc2c(NCc3cccs3)cc(Cl)nc2c1Br] | 2-[2-amino-3-(1H-1,2,3-triazol-4- yl)propyl]-3-bromo-5-chloro-7- thenylamino-1-oxa-4-azaindene |
| 80 | 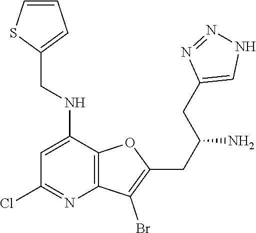 [SMILES: N[C@@H](Cc1c[nH]nn1)Cc1oc2c(NCc3cccs3)cc(Cl)nc2c1Br] | 2-[(S)-2-amino-3-(1H-1,2,3-triazol-4- yl)propyl]-3-bromo-5-chloro-7- thenylamino-1-oxa-4-azaindene |
| 81 | 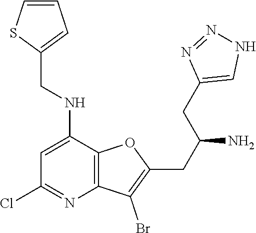 [SMILES: N[C@H](Cc1c[nH]nn1)Cc1oc2c(NCc3cccs3)cc(Cl)nc2c1Br] | 2-[(R)-2-amino-3-(1H-1,2,3-triazol-4- yl)propyl]-3-bromo-5-chloro-7- thenylamino-1-oxa-4-azaindene |
| 82 | 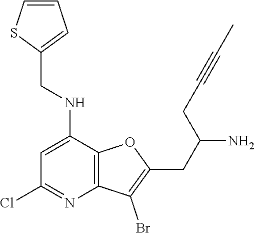 [SMILES: CC#CCC(N)Cc1oc2c(NCc3cccs3)cc(Cl)nc2c1Br] | 2-(2-amino-4-hexynyl)-3-bromo-5-chloro- 7-thenylamino-1-oxa-4-azaindene |
| 83 | 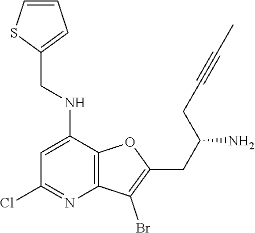 [SMILES: CC#CC[C@H](N)Cc1oc2c(NCc3cccs3)cc(Cl)nc2c1Br] | 2-[(S)-2-amino-4-hexynyl]-3-bromo-5- chloro-7-thenylamino-1-oxa-4-azaindene |
| 84 | 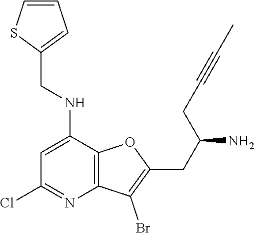 [SMILES: CC#CC[C@@H](N)Cc1oc2c(NCc3cccs3)cc(Cl)nc2c1Br] | 2-[(R)-2-amino-4-hexynyl]-3-bromo-5- chloro-7-thenylamino-1-oxa-4-azaindene |
| 85 | 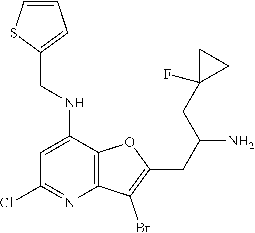 [SMILES: NC(Cc1oc2c(NCc3cccs3)cc(Cl)nc2c1Br)CC1(F)CC1] | 2-[2-amino-3-(1- fluorocyclopropyl)propyl]-3-bromo-5- chloro-7-thenylamino-1-oxa-4-azaindene |
| 86 | 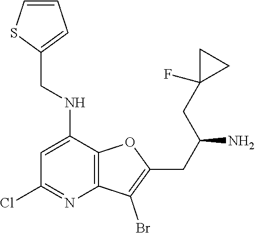 [SMILES: N[C@@H](Cc1oc2c(NCc3cccs3)cc(Cl)nc2c1Br)CC1(F)CC1] | 2-[(S)-2-amino-3-(1- fluorocyclopropyl)propyl]-3-bromo-5- chloro-7-thenylamino-1-oxa-4-azaindene |
| and | | |
| 87 | 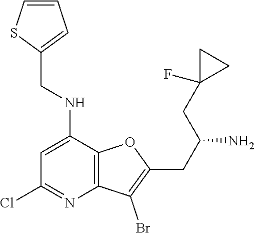 [SMILES: N[C@H](Cc1oc2c(NCc3cccs3)cc(Cl)nc2c1Br)CC1(F)CC1] | 2-[(R)-2-amino-3-(1- fluorocyclopropyl)propyl]-3-bromo-5- chloro-7-thenylamino-1-oxa-4-azaindene |
| 88 | 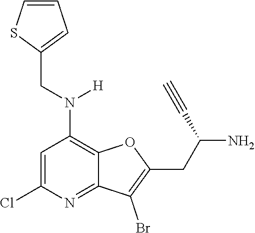 [SMILES: C#C[C@H](N)Cc1oc2c(NCc3cccs3)cc(Cl)nc2c1Br] | 2-[(R)-2-amino-3-butynyl]-3-bromo-5- chloro-7-thenylamino-1-oxa-4-azaindene |
| 89 | 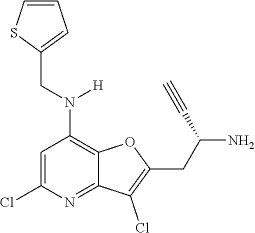 [SMILES: C#C[C@H](N)Cc1oc2c(NCc3cccs3)cc(Cl)nc2c1Cl] | 2-[(R)-2-amino-3-butynyl]-3,5-dichloro-7- thenylamino-1-oxa-4-azaindene |
| 90 | 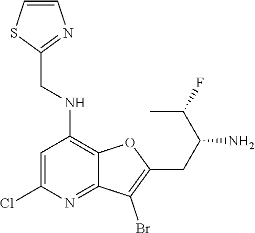 [SMILES: C[C@H](F)[C@H](N)Cc1oc2c(NCc3nccs3)cc(Cl)nc2c1Br] | 2-[(2R,3S)-2-amino-3-fluorobutyl]-3- bromo-5-chloro-7-{[(1,3-thiazol-2- yl)methyl]amino}-1-oxa-4-azaindene |
| 91 | 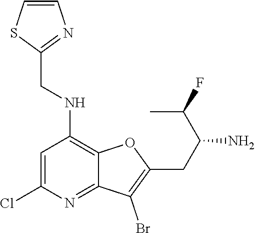 [SMILES: C[C@@H](F)[C@H](N)Cc1oc2c(NCc3nccs3)cc(Cl)nc2c1Br] | 2-[(2R,3R)-2-amino-3-fluorobutyl]-3- bromo-5-chloro-7-{[(1,3-thiazol-2- yl)methyl]amino}-1-oxa-4-azaindene |
| 92 | 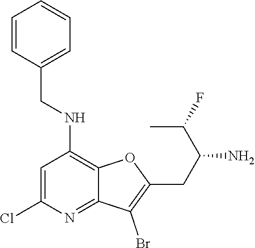 [SMILES: C[C@H](F)[C@H](N)Cc1oc2c(NCc3ccccc3)cc(Cl)nc2c1Br] | 2-[(2R,3S)-2-amino-3-fluorobutyl]-7- benzylamino-3-bromo-5-chloro-1-oxa-4- azaindene |
| 93 | 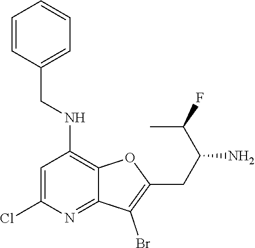 [SMILES: C[C@@H](F)[C@H](N)Cc1oc2c(NCc3ccccc3)cc(Cl)nc2c1Br] | 2-[(2R,3R)-2-amino-3-fluorobutyl]-7- benzylamino-3-bromo-5-chloro-1-oxa-4- azaindene |
| 94 | 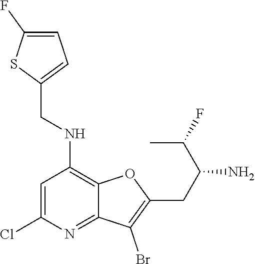 [SMILES: C[C@H](F)[C@H](N)Cc1oc2c(NCc3ccc(F)s3)cc(Cl)nc2c1Br] | 2-[(2R,3S)-2-amino-3-fluorobutyl]-3- bromo-5-chloro-7-{[(5-fluoro-2- thienyl)methyl]amino}-1-oxa-4-azaindene |
| 95 | 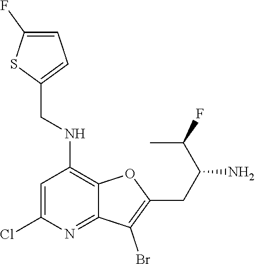 [SMILES: C[C@@H](F)[C@H](N)Cc1oc2c(NCc3ccc(F)s3)cc(Cl)nc2c1Br] | 2-[(2R,3R)-2-amino-3-fluorobutyl]-3- bromo-5-chloro-7-{[(5-fluoro-2- thienyl)methyl]amino}-1-oxa-4-azaindene |
| 96 | 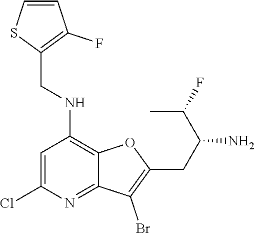 [SMILES: C[C@H](F)[C@H](N)Cc1oc2c(NCc3sccc3F)cc(Cl)nc2c1Br] | 2-[(2R,3S)-2-amino-3-fluorobutyl]-3- bromo-5-chloro-7-{[(3-fluoro-2- thienyl)methyl]amino}-1-oxa-4-azaindene |
| 97 | 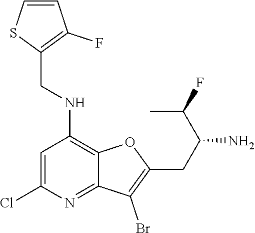 [SMILES: C[C@@H](F)[C@H](N)Cc1oc2c(NCc3sccc3F)cc(Cl)nc2c1Br] | 2-[(2R,3R)-2-amino-3-fluorobutyl]-3- bromo-5-chloro-7-{[(3-fluoro-2- thienyl)methyl]amino}-1-oxa-4-azaindene |
| 98 | 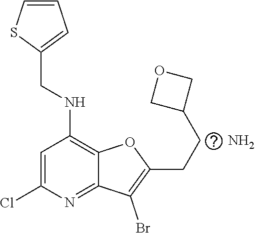 | 2-[(R)-2-amino-2-(3-oxetanyl)ethyl]-3- bromo-5-chloro-7-thenylamino-1-oxa-4- azaindene |
| 99 | 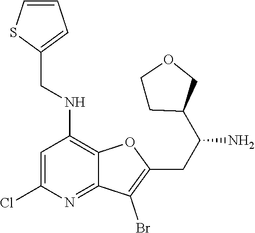 [SMILES: N[C@H](Cc1oc2c(NCc3cccs3)cc(Cl)nc2c1Br)[C@H]1CCOC1] | 2-{(R)-2-[(R)-tetrahydro-3-furyl]-2- aminoethyl}-3-bromo-5-chloro-7- thenylamino-1-oxa-4-azaindene |
| 100 | 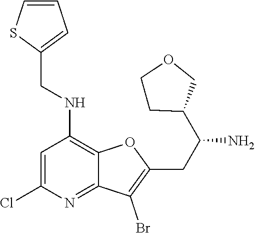 [SMILES: N[C@H](Cc1oc2c(NCc3cccs3)cc(Cl)nc2c1Br)[C@@H]1CCOC1] | 2-{(R)-2-[(S)-tetrahydro-3-furyl]-2- aminoethyl}-3-bromo-5-chloro-7- thenylamino-1-oxa-4-azaindene |
| and | | |
| 214 | 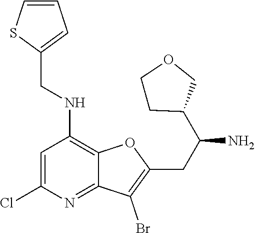 [SMILES: N[C@@H](Cc1oc2c(NCc3cccs3)cc(Cl)nc2c1Br)[C@@H]1CCOC1] | 2-{(S)-2-[(S)-tetrahydro-3-furyl]-2- aminoethyl}-3-bromo-5-chloro-7- thenylamino-1-oxa-4-azaindene |
| 215 | 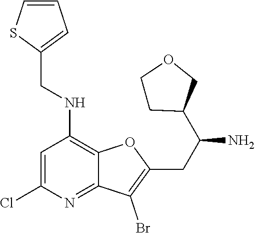 [SMILES: N[C@@H](Cc1oc2c(NCc3cccs3)cc(Cl)nc2c1Br)[C@H]1CCOC1] | 2-{(S)-2-[(R)-tetrahydro-3-furyl]-2- aminoethyl}-3-bromo-5-chloro-7- thenylamino-1-oxa-4-azaindene |
| 101 | 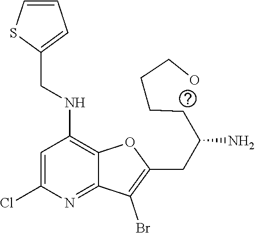 [SMILES: N[C@@H](CCCCO)Cc1oc2c(NCc3cccs3)cc(Cl)nc2c1Br] | 2-{(R)-2-[(S)-tetrahydro-2-furyl]-2- aminoethyl}-3-bromo-5-chloro-7- thenylamino-1-oxa-4-azaindene |
| 102 | 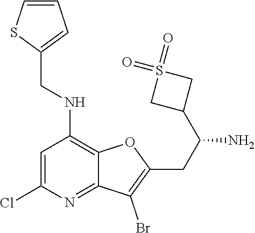 [SMILES: N[C@H](Cc1oc2c(NCc3cccs3)cc(Cl)nc2c1Br)C1CS(=O)(=O)C1] | 3-[(R)-1-amino-2-(3-bromo-5-chloro-7- thenylamino-1-oxa-4-aza-2- indenyl)ethyl]-1λ⁶-1,1-thietanedione |
| 103 | 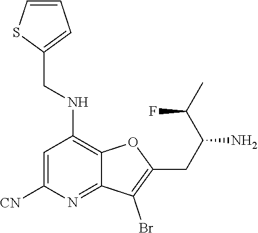 | 2-[(2R,3S)-2-amino-3-fluorobutyl]-3- bromo-7-thenylamino-1-oxa-4-aza-5- indenecarbonitrile |
| 104 | 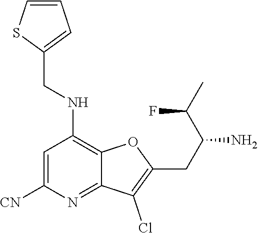 | 2-[(2R,3S)-2-amino-3-fluorobutyl]-3- chloro-7-thenylamino-1-oxa-4-aza-5- indenecarbonitrile |
| 105 | 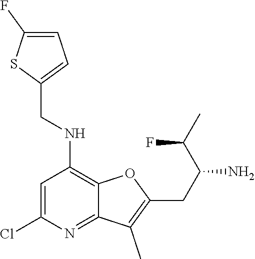 [SMILES: Cc1c(C[C@@H](N)[C@H](C)F)oc2c(NCc3ccc(F)s3)cc(Cl)nc12] | 2-[(2R,3S)-2-amino-3-fluorobutyl]-5- chloro-7-{[(5-fluoro-2- thienyl)methyl]amino}-3-methyl-1-oxa-4- azaindene |
| 106 | 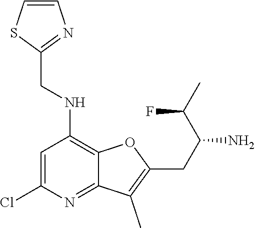 [SMILES: Cc1c(C[C@@H](N)[C@H](C)F)oc2c(NCc3nccs3)cc(Cl)nc12] | 2-[(2R,3S)-2-amino-3-fluorobutyl]-5- chloro-3-methyl-7-{[(1,3-thiazol-2- yl)methyl]amino}-1-oxa-4-azaindene |
| 107 | 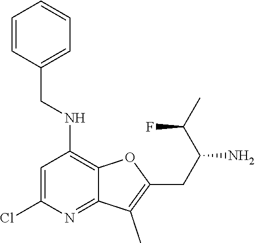 [SMILES: Cc1c(C[C@@H](N)[C@H](C)F)oc2c(NCc3ccccc3)cc(Cl)nc12] | 2-[(2R,3S)-2-amino-3-fluorobutyl]-7- benzylamino-5-chloro-3-methyl-1-oxa-4- azaindene |
| 108 | 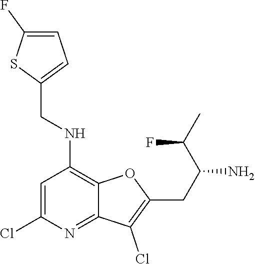 [SMILES: C[C@H](F)[C@H](N)Cc1oc2c(NCc3ccc(F)s3)cc(Cl)nc2c1Cl] | 2-[(2R,3S)-2-amino-3-fluorobutyl]-3,5- dichloro-7-{[(5-fluoro-2- thienyl)methyl]amino}-1-oxa-4-azaindene |
| 109 | 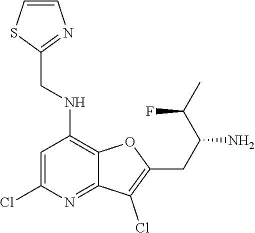 [SMILES: C[C@H](F)[C@H](N)Cc1oc2c(NCc3nccs3)cc(Cl)nc2c1Cl] | 2-[(2R,3S)-2-amino-3-fluorobutyl]-3,5- dichloro-7-{[(1,3-thiazol-2- yl)methyl]amino}-1-oxa-4-azaindene |
| 110 | 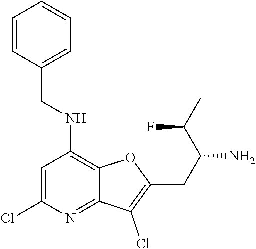 [SMILES: C[C@H](F)[C@H](N)Cc1oc2c(NCc3ccccc3)cc(Cl)nc2c1Cl] | 2-[(2R,3S)-2-amino-3-fluorobutyl]-7- benzylamino-3,5-dichloro-1-oxa-4- azaindene |
| 111 | 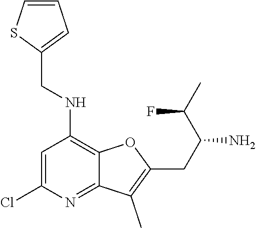 [SMILES: Cc1c(C[C@@H](N)[C@H](C)F)oc2c(NCc3cccs3)cc(Cl)nc12] | 2-[(2R,3S)-2-amino-3-fluorobutyl]-3- methyl-7-thenylamino-1-oxa-4-aza-5- indenecarbonitrile |
| 112 | 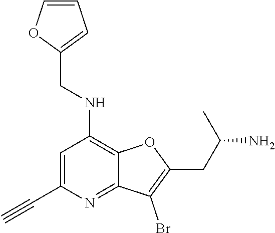 [SMILES: C#Cc1cc(NCc2ccco2)c2oc(C[C@H](C)N)c(Br)c2n1] | 2-[(S)-2-aminopropyl]-3-bromo-5-ethynyl- 7-(furfurylamino)-1-oxa-4-azaindene |
| 113 | 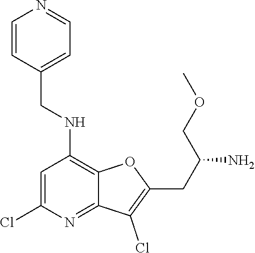 [SMILES: COC[C@H](N)Cc1oc2c(NCc3ccncc3)cc(Cl)nc2c1Cl] | 2-[(R)-2-amino-3-methoxypropyl]-3,5- dichloro-7-{[(4-pyridyl)methyl]amino}-1- oxa-4-azaindene |
| 114 | 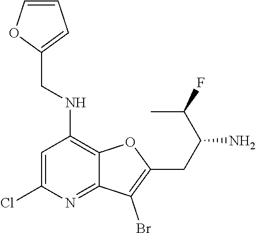 [SMILES: C[C@@H](F)[C@H](N)Cc1oc2c(NCc3ccco3)cc(Cl)nc2c1Br] | 2-[(2R,3R)-2-amino-3-fluorobutyl]-3- bromo-5-chloro-7-(furfurylamino)-1-oxa- 4-azaindene |
| 115 | 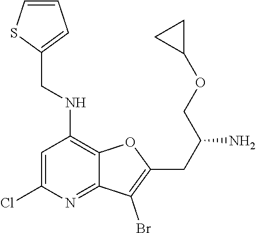 [SMILES: N[C@@H](COC1CC1)Cc1oc2c(NCc3cccs3)cc(Cl)nc2c1Br] | 2-[(R)-2-amino-3-cyclopropoxypropyl]-3- bromo-5-chloro-7-thenylamino-1-oxa-4- azaindene |
| 116 | 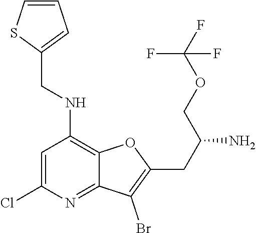 [SMILES: N[C@@H](COC(F)(F)F)Cc1oc2c(NCc3cccs3)cc(Cl)nc2c1Br] | 2-[(R)-2-amino-3- trifluoromethoxypropyl]-3-bromo-5- chloro-7-thenylamino-1-oxa-4-azaindene |
| 117 | 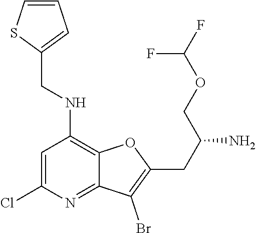 [SMILES: N[C@@H](COC(F)F)Cc1oc2c(NCc3cccs3)cc(Cl)nc2c1Br] | 2-[(R)-2-amino-3-difluoromethoxypropyl]- 3-bromo-5-chloro-7-thenylamino-1-oxa- 4-azaindene |
| 118 | 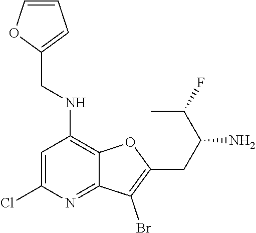 [SMILES: C[C@H](F)[C@H](N)Cc1oc2c(NCc3ccco3)cc(Cl)nc2c1Br] | 2-[(2R,3S)-2-amino-3-fluorobutyl]-3- bromo-5-chloro-7-(furfurylamino)-1-oxa- 4-azaindene |
| 119 | 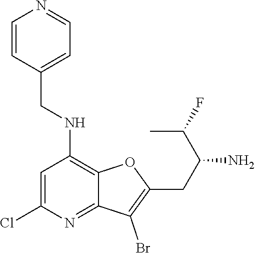 [SMILES: C[C@H](F)[C@H](N)Cc1oc2c(NCc3ccncc3)cc(Cl)nc2c1Br] | 2-[(2R,3S)-2-amino-3-fluorobutyl]-3- bromo-5-chloro-7-{[(4- pyridyl)methyl]amino}-1-oxa-4-azaindene |
| 120 | 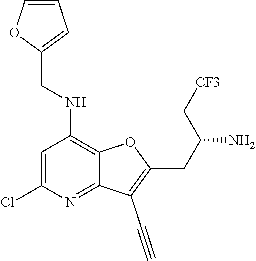 [SMILES: C#Cc1c(C[C@@H](N)CC(F)(F)F)oc2c(NCc3ccco3)cc(Cl)nc12] | 2-[(R)-2-amino-4,4,4-trifluorobutyl]-5- chloro-3-ethynyl-7-(furfurylamino)-1-oxa- 4-azaindene |
| 121 | 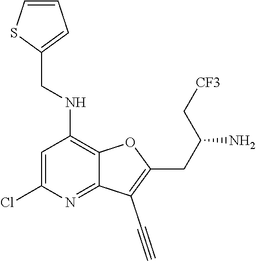 [SMILES: C#Cc1c(C[C@@H](N)CC(F)(F)F)oc2c(NCc3cccs3)cc(Cl)nc12] | 2-[(R)-2-amino-4,4,4-trifluorobutyl]-5- chloro-3-ethynyl-7-thenylamino-1-oxa-4- azaindene |
| 122 | 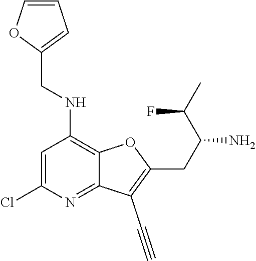 [SMILES: C#Cc1c(C[C@@H](N)[C@H](C)F)oc2c(NCc3ccco3)cc(Cl)nc12] | 2-[(2R,3S)-2-amino-3-fluorobutyl]-5- chloro-3-ethynyl-7-(furfurylamino)-1-oxa- 4-azaindene |
| 123 | 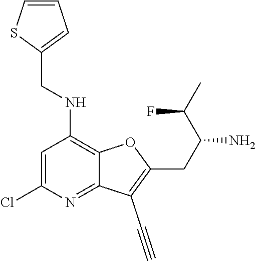 [SMILES: C#Cc1c(C[C@@H](N)[C@H](C)F)oc2c(NCc3cccs3)cc(Cl)nc12] | 2-[(2R,3S)-2-amino-3-fluorobutyl]-5- chloro-3-ethynyl-7-thenylamino-1-oxa-4- azaindene |
| 124 | 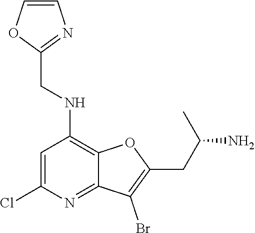 [SMILES: C[C@H](N)Cc1oc2c(NCc3ncco3)cc(Cl)nc2c1Br] | 2-[(S)-2-aminopropyl]-3-bromo-5-chloro- 7-{[(1,3-oxazol-2-yl)methyl]amino}-1-oxa- 4-azaindene |
| 125 | 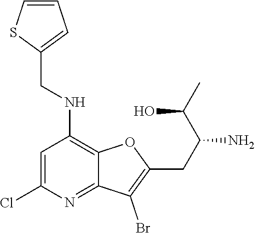 [SMILES: C[C@H](O)[C@H](N)Cc1oc2c(NCc3cccs3)cc(Cl)nc2c1Br] | (2S,3R)-3-amino-4-(3-bromo-5-chloro-7- thenylamino-1-oxa-4-aza-2-indenyl)-2- butanol |
| 126 | 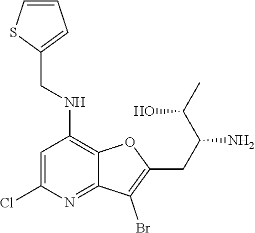 [SMILES: C[C@@H](O)[C@H](N)Cc1oc2c(NCc3cccs3)cc(Cl)nc2c1Br] | (2R,3R)-3-amino-4-(3-bromo-5-chloro-7- thenylamino-1-oxa-4-aza-2-indenyl)-2- butanol |
| 127 | 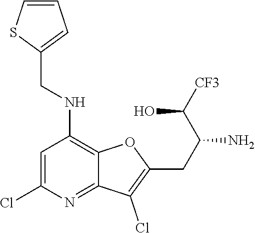 [SMILES: N[C@H](Cc1oc2c(NCc3cccs3)cc(Cl)nc2c1Cl)[C@@H](O)C(F)(F)F] | (2R,3R)-3-amino-4-(3-bromo-5-chloro-7- thenylamino-1-oxa-4-aza-2-indenyl)- 1,1,1-trifluoro-2-butanol |
| 128 | 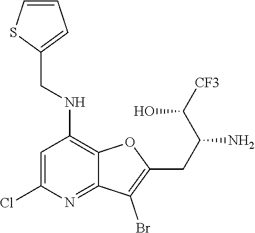 [SMILES: N[C@H](Cc1oc2c(NCc3cccs3)cc(Cl)nc2c1Br)[C@H](O)C(F)(F)F] | (2S,3R)-3-amino-4-(3-bromo-5-chloro-7- thenylamino-1-oxa-4-aza-2-indenyl)- 1,1,1-trifluoro-2-butanol |
| 129 | 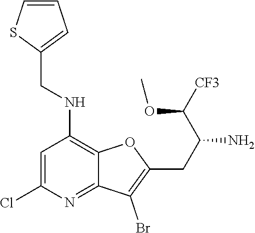 [SMILES: CO[C@H]([C@H](N)Cc1oc2c(NCc3cccs3)cc(Cl)nc2c1Br)C(F)(F)F] | 2-[(2R,3R)-2-amino-4,4,4-trifluoro-3- methoxybutyl]-3-bromo-5-chloro-7- thenylamino-1-oxa-4-azaindene |
| 130 | 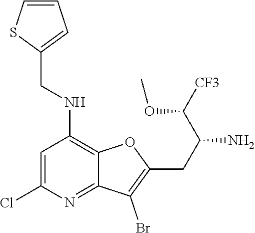 [SMILES: CO[C@@H]([C@H](N)Cc1oc2c(NCc3cccs3)cc(Cl)nc2c1Br)C(F)(F)F] | 2-[(2R,3S)-2-amino-4,4,4-trifluoro-3- methoxybutyl]-3-bromo-5-chloro-7- thenylamino-1-oxa-4-azaindene |
| 131 | 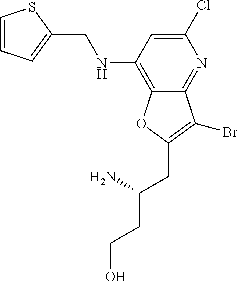 [SMILES: N[C@@H](CCO)Cc1oc2c(NCc3cccs3)cc(Cl)nc2c1Br] | (S)-3-amino-4-(3-bromo-5-chloro-7- thenylamino-1-oxa-4-aza-2-indenyl)-1- butanol |
| 132 | 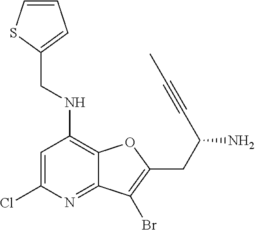 [SMILES: CC#C[C@H](N)Cc1oc2c(NCc3cccs3)cc(Cl)nc2c1Br] | 2-[(R)-2-amino-3-pentynyl]-3-bromo-5- chloro-7-thenylamino-1-oxa-4-azaindene |
| 133 | 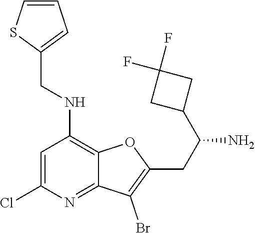 [SMILES: N[C@H](Cc1oc2c(NCc3cccs3)cc(Cl)nc2c1Br)C1CC(F)(F)C1] | 2-[(R)-2-amino-2-(3,3- difluorocyclobutyl)ethyl]-3-bromo-5- chloro-7-thenylamino-1-oxa-4-azaindene |
| 134 | 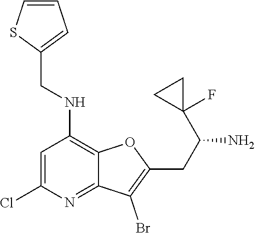 [SMILES: N[C@H](Cc1oc2c(NCc3cccs3)cc(Cl)nc2c1Br)C1(F)CC1] | 2-[(R)-2-amino-2-cyclopropylethyl]-3- bromo-5-chloro-7-thenylamino-1-oxa-4- azaindene |
| 135 | 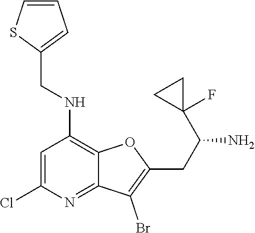 [SMILES: N[C@H](Cc1oc2c(NCc3cccs3)cc(Cl)nc2c1Br)C1(F)CC1] | 2-[(R)-2-amino-2-(1- fluorocyclopropyl)ethyl]-3-bromo-5- chloro-7-thenylamino-1-oxa-4-azaindene |
| 136 | 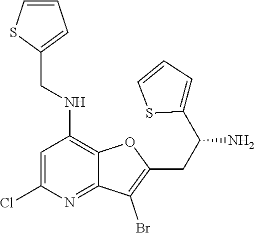 [SMILES: N[C@H](Cc1oc2c(NCc3cccs3)cc(Cl)nc2c1Br)c1cccs1] | 2-[(R)-2-amino-2-(2-thienyl)ethyl]-3- bromo-5-chloro-7-thenylamino-1-oxa-4- azaindene |
| 137 | 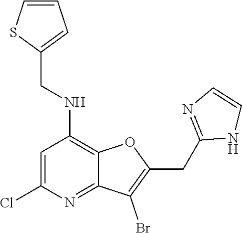 [SMILES: Clc1cc(NCc2cccs2)c2oc(Cc3ncc[nH]3)c(Br)c2n1] | (thenyl){3-bromo-5-chloro-2-[(2- imidazolyl)methyl]-1-oxa-4-aza-7- indenyl}amine |
| 138 | 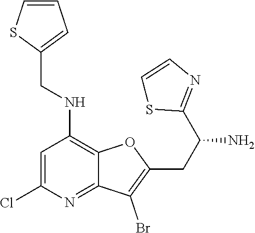 [SMILES: N[C@H](Cc1oc2c(NCc3cccs3)cc(Cl)nc2c1Br)c1nccs1] | 2-[(R)-2-amino-2-(1,3-thiazol-2-yl)ethyl]- 3-bromo-5-chloro-7-thenylamino-1-oxa- 4-azaindene |
| 139 | 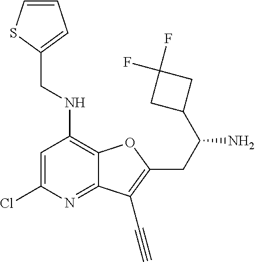 [SMILES: C#Cc1c(C[C@@H](N)C2CC(F)(F)C2)oc2c(NCc3cccs3)cc(Cl)nc12] | 2-[(R)-2-amino-2-(3,3- difluorocyclobutyl)ethyl]-5-chloro-3- ethynyl-7-thenylamino-1-oxa-4-azaindene |
| 140 | 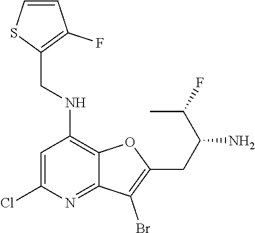 [SMILES: C[C@H](F)[C@H](N)Cc1oc2c(NCc3sccc3F)cc(Cl)nc2c1Br] | 2-[(2R,3S)-2-amino-3-fluorobutyl]-3,5- dichloro-7-{[(3-fluoro-2- thienyl)methyl]amino}-1-oxa-4-azaindene |
| 141 | 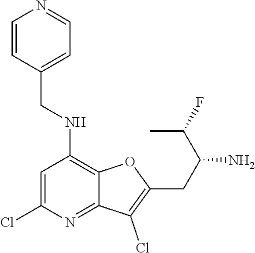 [SMILES: C[C@H](F)[C@H](N)Cc1oc2c(NCc3ccncc3)cc(Cl)nc2c1Cl] | 2-[(2R,3S)-2-amino-3-fluorobutyl]-3,5- dichloro-7-{[(4-pyridyl)methyl]amino}-1- oxa-4-azaindene |
| 142 | 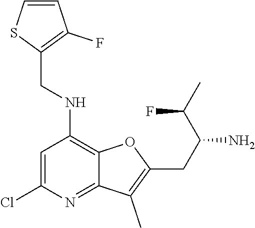 [SMILES: Cc1c(C[C@@H](N)[C@H](C)F)oc2c(NCc3sccc3F)cc(Cl)nc12] | 2-[(2R,3S)-2-amino-3-fluorobutyl]-5- chloro-7-{[(3-fluoro-2- thienyl)methyl]amino}-3-methyl-1-oxa-4- azaindene |
| 143 | 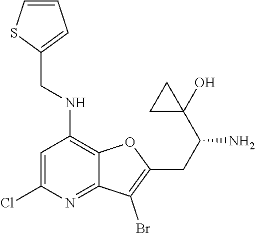 [SMILES: N[C@H](Cc1oc2c(NCc3cccs3)cc(Cl)nc2c1Br)C1(O)CC1] | 1-[(R)-1-amino-2-(3-bromo-5-chloro-7- thenylamino-1-oxa-4-aza-2- indenyl)ethyl]cyclopropanol |
| 144 | 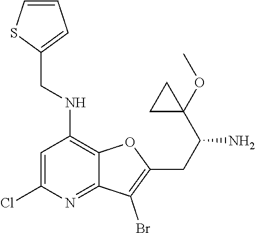 [SMILES: COC1([C@H](N)Cc2oc3c(NCc4cccs4)cc(Cl)nc3c2Br)CC1] | 2-[(R)-2-amino-2-(1- methoxycyclopropyl)ethyl]-3-bromo-5- chloro-7-thenylamino-1-oxa-4-azaindene |
| 145 | 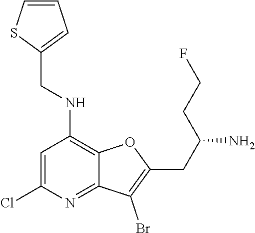 [SMILES: N[C@@H](CCF)Cc1oc2c(NCc3cccs3)cc(Cl)nc2c1Br] | 2-[(R)-2-amino-4-fluorobutyl]-3-bromo-5- chloro-7-thenylamino-1-oxa-4-azaindene |
| 146 | 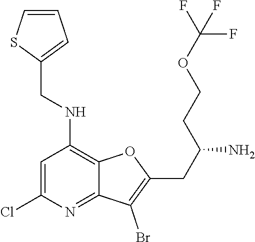 [SMILES: N[C@@H](CCOC(F)(F)F)Cc1oc2c(NCc3cccs3)cc(Cl)nc2c1Br] | 2-[(S)-2-amino-4-trifluoromethoxybutyl]- 3-bromo-5-chloro-7-thenylamino-1-oxa- 4-azaindene |
| 147 | 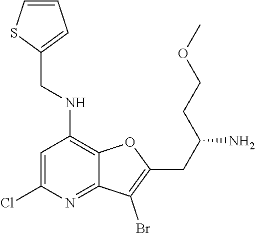 [SMILES: COCC[C@H](N)Cc1oc2c(NCc3cccs3)cc(Cl)nc2c1Br] | 2-[(S)-2-amino-4-methoxybutyl]-3-bromo- 5-chloro-7-thenylamino-1-oxa-4- azaindene |
| 148 | 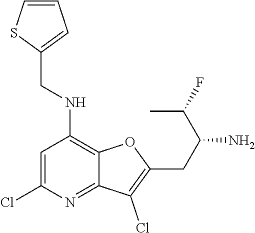 [SMILES: C[C@H](F)[C@H](N)Cc1oc2c(NCc3cccs3)cc(Cl)nc2c1Cl] | 2-[(2R,3S)-2-amino-3-fluorobutyl]-3,5- dichloro-7-thenylamino-1-oxa-4- azaindene |
| 149 | 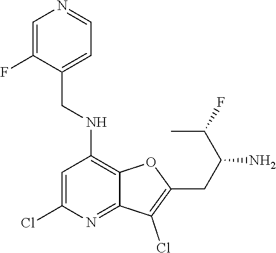 [SMILES: C[C@H](F)[C@H](N)Cc1oc2c(NCc3ccncc3F)cc(Cl)nc2c1Cl] | 2-[(2R,3S)-2-amino-3-fluorobutyl]-3,5- dichloro-7-{[(3-fluoro-4- pyridyl)methyl]amino}-1-oxa-4-azaindene |
| 150 | 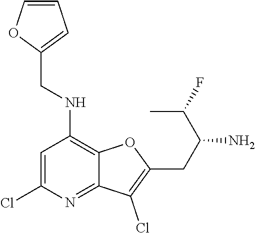 [SMILES: C[C@H](F)[C@H](N)Cc1oc2c(NCc3ccco3)cc(Cl)nc2c1Cl] | 2-[(2R,3S)-2-amino-3-fluorobutyl]-3,5- dichloro-7-(furfurylamino)-1-oxa-4- azaindene |
| 151 | 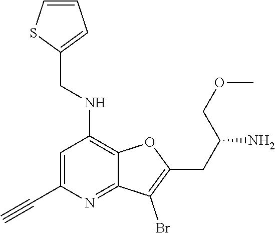 [SMILES: C#Cc1cc(NCc2cccs2)c2oc(C[C@@H](N)COC)c(Br)c2n1] | 2-[(R)-2-amino-3-methoxypropyl]-3- bromo-5-ethynyl-7-thenylamino-1-oxa-4- azaindene |
| 152 | 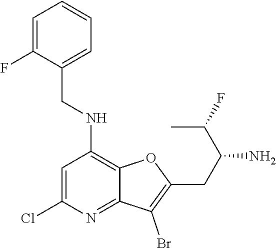 [SMILES: C[C@H](F)[C@H](N)Cc1oc2c(NCc3ccccc3F)cc(Cl)nc2c1Br] | 2-[(2R,3S)-2-amino-3-fluorobutyl]-3- bromo-5-chloro-7-{[(o- fluorophenyl)methyl]amino}-1-oxa-4- azaindene |
| 153 | 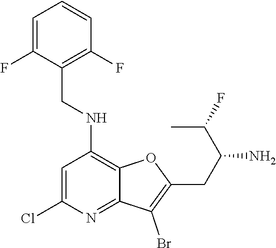 [SMILES: C[C@H](F)[C@H](N)Cc1oc2c(NCc3c(F)cccc3F)cc(Cl)nc2c1Br] | 2-[(2R,3S)-2-amino-3-fluorobutyl]-3- bromo-5-chloro-7-{[(2,6- difluorophenyl)methyl]amino}-1-oxa-4- azaindene |
| 154 | 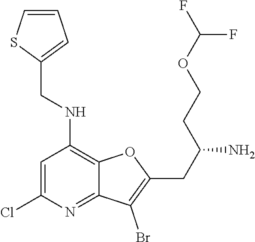 [SMILES: N[C@@H](CCOC(F)F)Cc1oc2c(NCc3cccs3)cc(Cl)nc2c1Br] | 2-[(S)-2-amino-4-difluoromethoxybutyl]- 3-bromo-5-chloro-7-thenylamino-1-oxa- 4-azaindene |
| 155 | 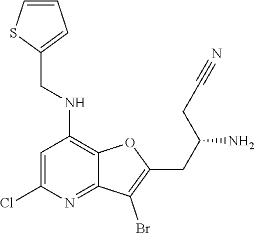 [SMILES: N#CC[C@H](N)Cc1oc2c(NCc3cccs3)cc(Cl)nc2c1Br] | (S)-3-amino-4-(3-bromo-5-chloro-7- thenylamino-1-oxa-4-aza-2- indenyl)butyronitrile |
| 156 | 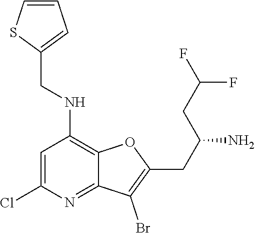 [SMILES: N[C@H](Cc1oc2c(NCc3cccs3)cc(Cl)nc2c1Br)CC(F)F] | 2-[(R)-2-amino-4,4-difluorobutyl]-3- bromo-5-chloro-7-thenylamino-1-oxa-4- azaindene |
| 157 | 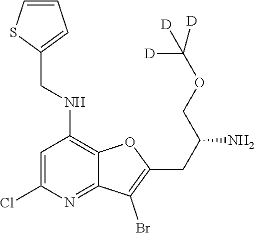 [SMILES: [2H]C([2H])([2H])OC[C@H](N)Cc1oc2c(NCc3cccs3)cc(Cl)nc2c1Br] | 2-[(R)-2-amino-3-(2H3)methoxypropyl]-3- bromo-5-chloro-7-thenylamino-1-oxa-4- azaindene |
| 158 | 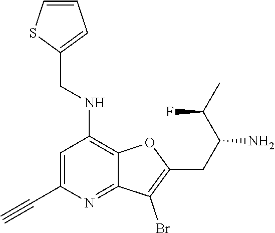 [SMILES: C#Cc1cc(NCc2cccs2)c2oc(C[C@@H](N)[C@H](C)F)c(Br)c2n1] | 2-[(2R,3S)-2-amino-3-fluorobutyl]-3- bromo-5-ethynyl-7-thenylamino-1-oxa-4- azaindene |
| 159 | 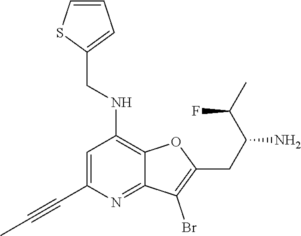 [SMILES: CC#Cc1cc(NCc2cccs2)c2oc(C[C@@H](N)[C@H](C)F)c(Br)c2n1] | 2-[(2R,3S)-2-amino-3-fluorobutyl]-3- bromo-5-(1-propynyl)-7-thenylamino-1- oxa-4-azaindene |
| 160 | 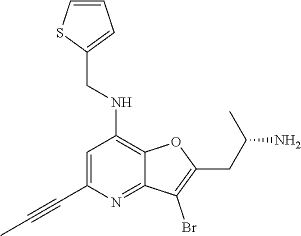 [SMILES: CC#Cc1cc(NCc2cccs2)c2oc(C[C@H](C)N)c(Br)c2n1] | 2-[(S)-2-aminopropyl]-3-bromo-5-(1- propynyl)-7-thenylamino-1-oxa-4- azaindene |
| 161 | 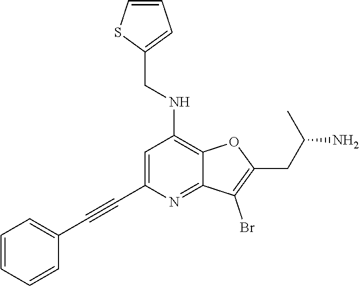 [SMILES: C[C@H](N)Cc1oc2c(NCc3cccs3)cc(C#Cc3ccccc3)nc2c1Br] | 2-[(S)-2-aminopropyl]-3-bromo-5-(2- phenylethynyl)-7-thenylamino-1-oxa-4- azaindene |
| 162 | 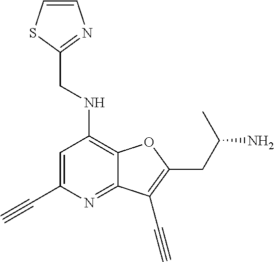 [SMILES: C#Cc1cc(NCc2nccs2)c2oc(C[C@H](C)N)c(C#C)c2n1] | 2-[(S)-2-aminopropyl]-3,5-diethynyl-7- thenylamino-1-oxa-4-azaindene |
| 163 | 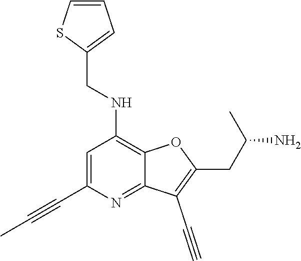 [SMILES: C#Cc1c(C[C@H](C)N)oc2c(NCc3cccs3)cc(C#CC)nc12] | 2-[(S)-2-aminopropyl]-3-ethynyl-5-(1- propynyl)-7-thenylamino-1-oxa-4- azaindene |
| 164 | 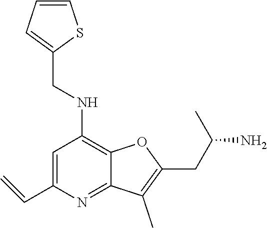 [SMILES: C=Cc1cc(NCc2cccs2)c2oc(C[C@H](C)N)c(C)c2n1] | 2-[(S)-2-aminopropyl]-3-bromo-7- thenylamino-5-vinyl-1-oxa-4-azaindene |
| 165 | 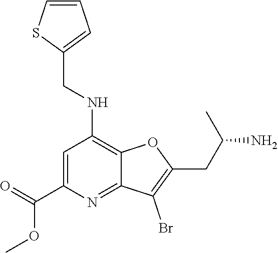 [SMILES: COC(=O)c1cc(NCc2cccs2)c2oc(C[C@H](C)N)c(Br)c2n1] | methyl 2-[(S)-2-aminopropyl]-3-bromo-7- thenylamino-1-oxa-4-aza-5- indenecarboxylate |
| 167 | 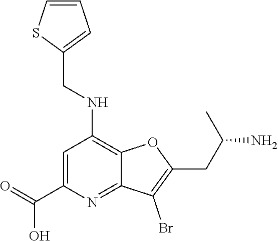 [SMILES: C[C@H](N)Cc1oc2c(NCc3cccs3)cc(C(=O)O)nc2c1Br] | 2-[(S)-2-aminopropyl]-3-bromo-7- thenylamino-1-oxa-4-aza-5- indenecarboxylic acid |
| 168 | 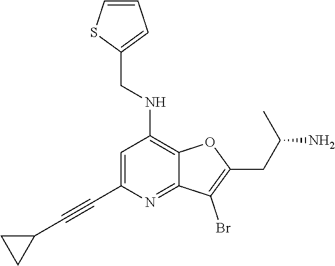 [SMILES: C[C@H](N)Cc1oc2c(NCc3cccs3)cc(C#CC3CC3)nc2c1Br] | 2-[(S)-2-aminopropyl]-3-bromo-5-(2- cyclopropylethynyl)-7-thenylamino-1-oxa- 4-azaindene |
| 169 | 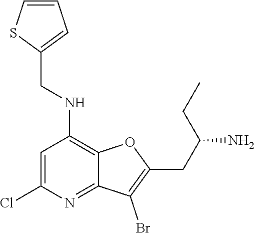 [SMILES: CC[C@H](N)Cc1oc2c(NCc3cccs3)cc(Cl)nc2c1Br] | 2-[(S)-2-aminobutyl]-3-bromo-5-chloro-7- thenylamino-1-oxa-4-azaindene |
| 170 | 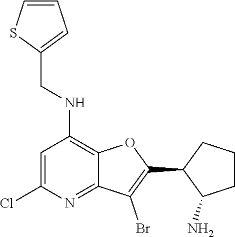 [SMILES: N[C@H]1CCC[C@@H]1c1oc2c(NCc3cccs3)cc(Cl)nc2c1Br] | 2-[(1S,2S)-2-aminocyclopentyl]-3-bromo- 5-chloro-7-thenylamino-1-oxa-4- azaindene |
| 171 | 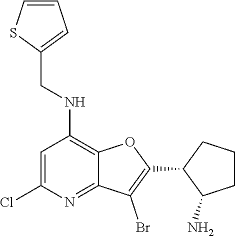 [SMILES: N[C@H]1CCC[C@H]1c1oc2c(NCc3cccs3)cc(Cl)nc2c1Br] | 2-[(1R,2S)-2-aminocyclopentyl]-3-bromo- 5-chloro-7-thenylamino-1-oxa-4- azaindene |
| 172 | 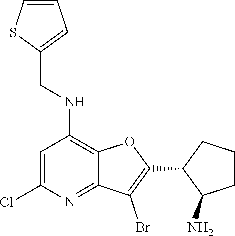 [SMILES: N[C@@H]1CCC[C@H]1c1oc2c(NCc3cccs3)cc(Cl)nc2c1Br] | 2-[(1R,2R)-2-aminocyclopentyl]-3-bromo- 5-chloro-7-thenylamino-1-oxa-4- azaindene |
| 173 | 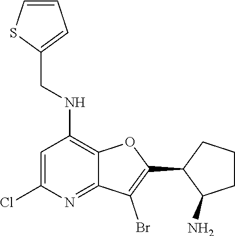 [SMILES: N[C@@H]1CCC[C@@H]1c1oc2c(NCc3cccs3)cc(Cl)nc2c1Br] | 2-[(1S,2R)-2-aminocyclopentyl]-3-bromo- 5-chloro-7-thenylamino-1-oxa-4- azaindene |
| 174 | 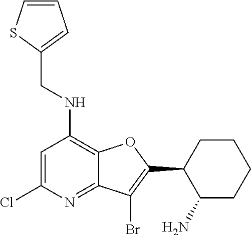 [SMILES: N[C@H]1CCCC[C@@H]1c1oc2c(NCc3cccs3)cc(Cl)nc2c1Br] | 2-[(1,2S)-2-aminocyclohexyl]-3-bromo-5- chloro-7-thenylamino-1-oxa-4-azaindene |
| 175 | 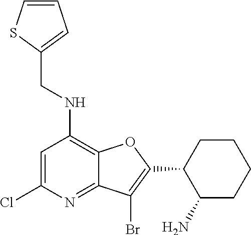 [SMILES: N[C@H]1CCCC[C@H]1c1oc2c(NCc3cccs3)cc(Cl)nc2c1Br] | 2-[(1R,2S)-2-aminocyclohexyl]-3-bromo- 5-chloro-7-thenylamino-1-oxa-4- azaindene |
| 176 | 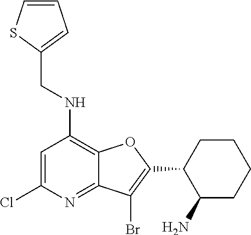 [SMILES: N[C@@H]1CCCC[C@H]1c1oc2c(NCc3cccs3)cc(Cl)nc2c1Br] | 2-[(1R,2R)-2-aminocyclohexyl]-3-bromo- 5-chloro-7-thenylamino-1-oxa-4- azaindene |
| 177 | 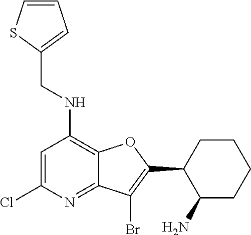 [SMILES: N[C@@H]1CCCC[C@@H]1c1oc2c(NCc3cccs3)cc(Cl)nc2c1Br] | 2-[(1S,2R)-2-aminocyclohexyl]-3-bromo- 5-chloro-7-thenylamino-1-oxa-4- azaindene |
| 178 | 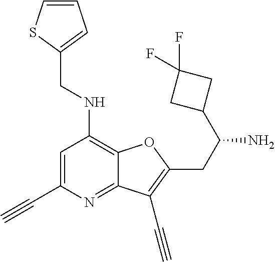 [SMILES: C#Cc1cc(NCc2cccs2)c2oc(C[C@@H](N)C3CC(F)(F)C3)c(C#C)c2n1] | 2-[(R)-2-amino-2-(3,3- difluorocyclobutyl)ethyl]-3,5-diethynyl-7- thenylamino-1-oxa-4-azaindene |
| 180 | 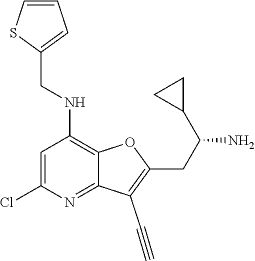 [SMILES: C#Cc1c(C[C@@H](N)C2CC2)oc2c(NCc3cccs3)cc(Cl)nc12] | 2-[(R)-2-amino-2-cyclopropylethyl]-5- chloro-3-ethynyl-7-thenylamino-1-oxa-4- azaindene |
| 181 | 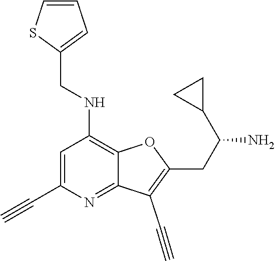 [SMILES: C#Cc1cc(NCc2cccs2)c2oc(C[C@@H](N)C3CC3)c(C#C)c2n1] | 2-[(R)-2-amino-2-cyclopropylethyl]-3,5- diethynyl-7-thenylamino-1-oxa-4- azaindene |
| 182 | 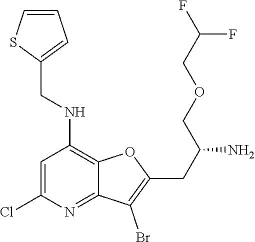 [SMILES: N[C@@H](COCC(F)F)Cc1oc2c(NCc3cccs3)cc(Cl)nc2c1Br] | 2-[(R)-2-amino-3-(2,2- difluoroethoxy)propyl]-3-bromo-5-chloro- 7-thenylamino-1-oxa-4-azaindene |
| 183 | 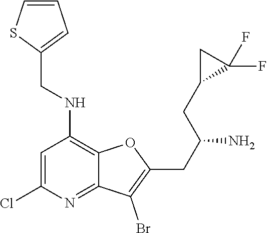 [SMILES: N[C@H](Cc1oc2c(NCc3cccs3)cc(Cl)nc2c1Br)C[C@@H]1CC1(F)F] | 2-{(S)-3-[(R)-2,2-difluorocyclopropyl]-2- aminopropyl}-3-bromo-5-chloro-7- thenylamino-1-oxa-4-azaindene |
| 184 | 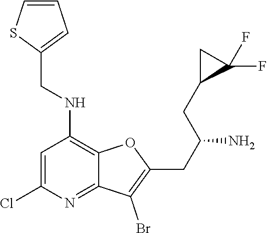 [SMILES: N[C@H](Cc1oc2c(NCc3cccs3)cc(Cl)nc2c1Br)C[C@H]1CC1(F)F] | 2-{(S)-3-[(S)-2,2-difluorocyclopropyl]-2- aminopropyl}-3-bromo-5-chloro-7- thenylamino-1-oxa-4-azaindene |
| 185 | 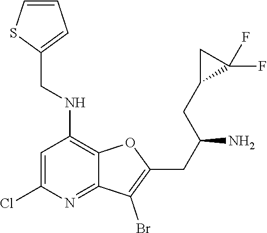 [SMILES: N[C@@H](Cc1oc2c(NCc3cccs3)cc(Cl)nc2c1Br)C[C@@H]1CC1(F)F] | 2-{(R)-3-[(R)-2,2-difluorocyclopropyl]-2- aminopropyl}-3-bromo-5-chloro-7- thenylamino-1-oxa-4-azaindene |
| and | | |
| 186 | 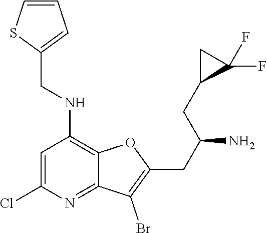 [SMILES: N[C@@H](Cc1oc2c(NCc3cccs3)cc(Cl)nc2c1Br)C[C@H]1CC1(F)F] | 2-{(R)-3-[(S)-2,2-difluorocyclopropyl]-2- aminopropyl}-3-bromo-5-chloro-7- thenylamino-1-oxa-4-azaindene |
| 187 | 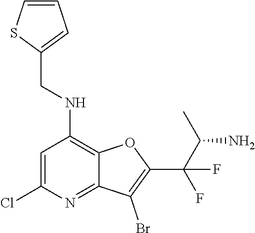 [SMILES: C[C@H](N)C(F)(F)c1oc2c(NCc3cccs3)cc(Cl)nc2c1Br] | 2-[(S)-2-amino-1,1-difluoropropyl]-3- bromo-5-chloro-7-thenylamino-1-oxa-4- azaindene |
| 188 | 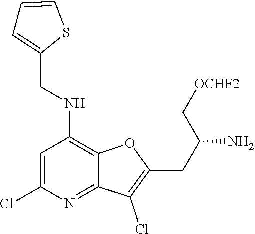 [SMILES: N[C@@H](COC(F)F)Cc1oc2c(NCc3cccs3)cc(Cl)nc2c1Cl] | 2-[(R)-2-amino-3-difluoromethoxypropyl]- 3,5-dichloro-7-thenylamino-1-oxa-4- azaindene |
| 189 | 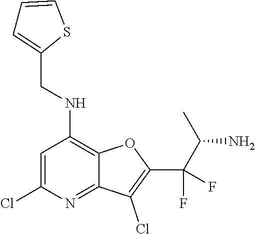 [SMILES: C[C@H](N)C(F)(F)c1oc2c(NCc3cccs3)cc(Cl)nc2c1Cl] | 2-[(S)-2-amino-1,1-difluoropropyl]-3,5- dichloro-7-thenylamino-1-oxa-4- azaindene |
| 190 | 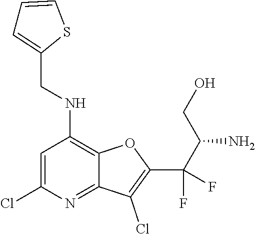 [SMILES: N[C@@H](CO)C(F)(F)c1oc2c(NCc3cccs3)cc(Cl)nc2c1Cl] | (S)-2-amino-3-(3,5-dichloro-7- thenylamino-1-oxa-4-aza-2-indenyl)-3,3- difluoro-1-propanol |
| 191 | 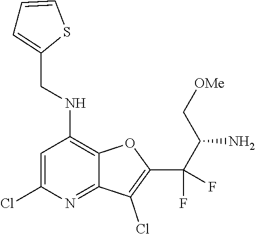 [SMILES: COC[C@H](N)C(F)(F)c1oc2c(NCc3cccs3)cc(Cl)nc2c1Cl] | 2-[(S)-2-amino-1,1-difluoro-3- methoxypropyl]-3,5-dichloro-7- thenylamino-1-oxa-4-azaindene |
| 192 | 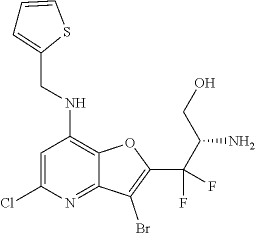 [SMILES: N[C@@H](CO)C(F)(F)c1oc2c(NCc3cccs3)cc(Cl)nc2c1Br] | (S)-2-amino-3-(3-bromo-5-chloro-7- thenylamino-1-oxa-4-aza-2-indenyl)-3,3- difluoro-1-propanol |
| 193 | 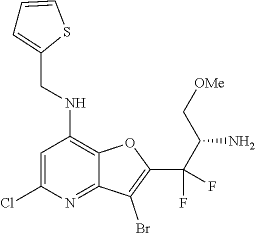 [SMILES: COC[C@H](N)C(F)(F)c1oc2c(NCc3cccs3)cc(Cl)nc2c1Br] | 2-[(S)-2-amino-1,1-difluoro-3- methoxypropyl]-3-bromo-5-chloro-7- thenylamino-1-oxa-4-azaindene |
| 194 | 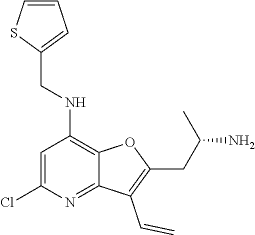 [SMILES: C=Cc1c(C[C@H](C)N)oc2c(NCc3cccs3)cc(Cl)nc12] | 2-[(S)-2-aminopropyl]-5-chloro-7- thenylamino-3-vinyl-1-oxa-4-azaindene |
| 195 | 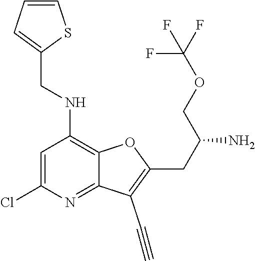 [SMILES: C#Cc1c(C[C@@H](N)COC(F)(F)F)oc2c(NCc3cccs3)cc(Cl)nc12] | 2-[(R)-2-amino-3- trifluoromethoxypropyl]-5-chloro-3- ethynyl-7-thenylamino-1-oxa-4-azaindene |
| 196 | 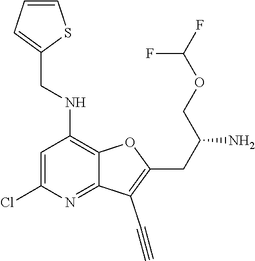 [SMILES: C#Cc1c(C[C@@H](N)COC(F)F)oc2c(NCc3cccs3)cc(Cl)nc12] | 2-[(R)-2-amino-3-difluoromethoxypropyl]- 5-chloro-3-ethynyl-7-thenylamino-1-oxa- 4-azaindene |
| 197 | 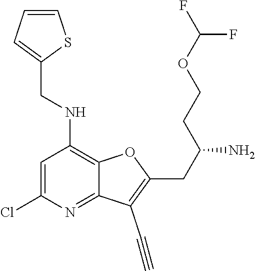 [SMILES: C#Cc1c(C[C@@H](N)CCOC(F)F)oc2c(NCc3cccs3)cc(Cl)nc12] | 2-[(S)-2-amino-4-difluoromethoxybutyl]- 5-chloro-3-ethynyl-7-thenylamino-1-oxa- 4-azaindene |
| 198 | 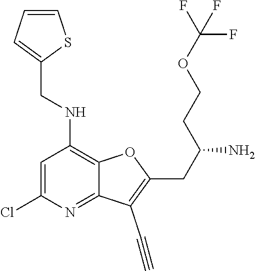 [SMILES: C#Cc1c(C[C@@H](N)CCOC(F)(F)F)oc2c(NCc3cccs3)cc(Cl)nc12] | 2-[(S)-2-amino-4-trifluoromethoxybutyl]- 5-chloro-3-ethynyl-7-thenylamino-1-oxa- 4-azaindene |
| 199 | 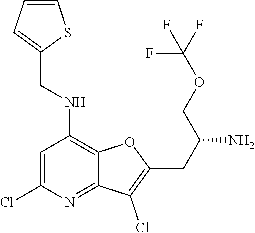 [SMILES: N[C@@H](COC(F)(F)F)Cc1oc2c(NCc3cccs3)cc(Cl)nc2c1Cl] | 2-[(R)-2-amino-3- trifluoromethoxypropyl]-3,5-dichloro-7- thenylamino-1-oxa-4-azaindene |
| 200 | 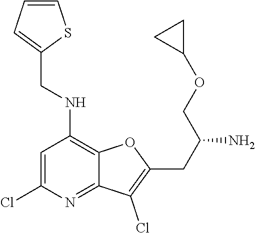 [SMILES: N[C@@H](COC1CC1)Cc1oc2c(NCc3cccs3)cc(Cl)nc2c1Cl] | 2-[(R)-2-amino-3-cyclopropoxypropyl]- 3,5-dichloro-7-thenylamino-1-oxa-4- azaindene |
| 201 | 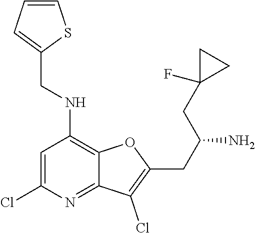 [SMILES: N[C@H](Cc1oc2c(NCc3cccs3)cc(Cl)nc2c1Cl)CC1(F)CC1] | 2-[(R)-2-amino-3-(1- fluorocyclopropyl)propyl]-3,5-dichloro-7- thenylamino-1-oxa-4-azaindene |
| 202 | 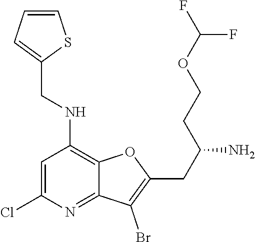 [SMILES: N[C@@H](CCOC(F)F)Cc1oc2c(NCc3cccs3)cc(Cl)nc2c1Br] | 2-[(S)-2-amino-4-difluoromethoxybutyl]- 3,5-dichloro-7-thenylamino-1-oxa-4- azaindene |
| 203 | 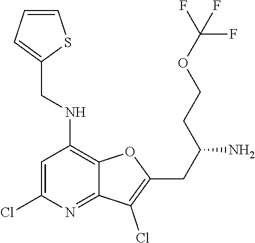 [SMILES: N[C@@H](CCOC(F)(F)F)Cc1oc2c(NCc3cccs3)cc(Cl)nc2c1Cl] | 2-[(S)-2-amino-4-trifluoromethoxybutyl]- 3,5-dichloro-7-thenylamino-1-oxa-4- azaindene |
| 204 | 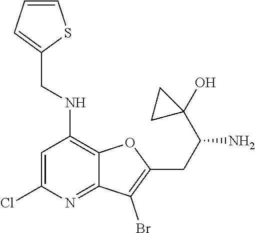 [SMILES: N[C@H](Cc1oc2c(NCc3cccs3)cc(Cl)nc2c1Br)C1(O)CC1] | 1-[(R)-1-amino-2-(3,5-dichloro-7- thenylamino-1-oxa-4-aza-2- indenyl)ethyl]cyclopropanol |
| 205 | 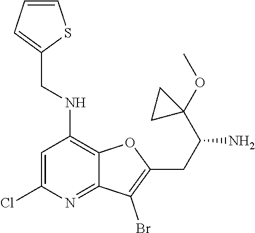 [SMILES: COC1([C@H](N)Cc2oc3c(NCc4cccs4)cc(Cl)nc3c2Br)CC1] | 2-[(R)-2-amino-2-(1- methoxycyclopropyl)ethyl]-3,5-dichloro- 7-thenylamino-1-oxa-4-azaindene |
| 206 | 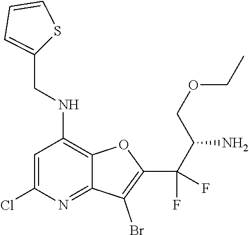 [SMILES: CCOC[C@H](N)C(F)(F)c1oc2c(NCc3cccs3)cc(Cl)nc2c1Br] | 2-[(S)-2-amino-3-ethoxy-1,1- difluoropropyl]-3-bromo-5-chloro-7- thenylamino-1-oxa-4-azaindene |
| 207 | 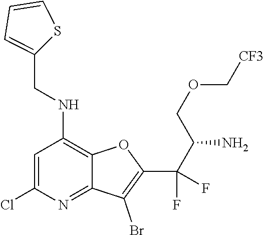 [SMILES: N[C@@H](COCC(F)(F)F)C(F)(F)c1oc2c(NCc3cccs3)cc(Cl)nc2c1Br] | 2-[(S)-2-amino-1,1-difluoro-3-(2,2,2- trifluoroethoxy)propyl]-3-bromo-5- chloro-7-thenylamino-1-oxa-4-azaindene |
| 208 | 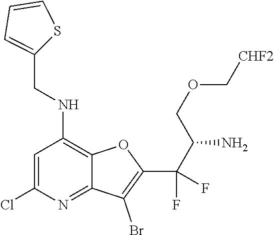 [SMILES: N[C@@H](COCC(F)F)C(F)(F)c1oc2c(NCc3cccs3)cc(Cl)nc2c1Br] | 2-[(S)-2-amino-3-(2,2-difluoroethoxy)-1,1- difluoropropyl]-3-bromo-5-chloro-7- thenylamino-1-oxa-4-azaindene |
| 209 | 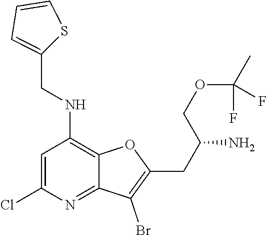 [SMILES: CC(F)(F)OC[C@H](N)Cc1oc2c(NCc3cccs3)cc(Cl)nc2c1Br] | 2-[(R)-2-amino-3-(1,1- difluoroethoxy)propyl]-3-bromo-5-chloro- 7-thenylamino-1-oxa-4-azaindene |
| 210 | 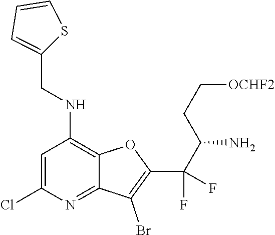 [SMILES: N[C@@H](CCOC(F)F)C(F)(F)c1oc2c(NCc3cccs3)cc(Cl)nc2c1Br] | 2-[(S)-2-amino-4-difluoromethoxy-1,1- difluorobutyl]-3-bromo-5-chloro-7- thenylamino-1-oxa-4-azaindene |
| 211 | 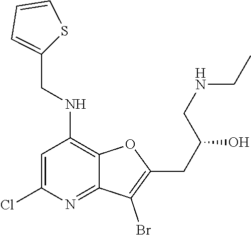 [SMILES: CCNC[C@H](O)Cc1oc2c(NCc3cccs3)cc(Cl)nc2c1Br] | (R)-1-(3-bromo-5-chloro-7-thenylamino- 1-oxa-4-aza-2-indenyl)-3-(ethylamino)-2- propanol |
| 212 | 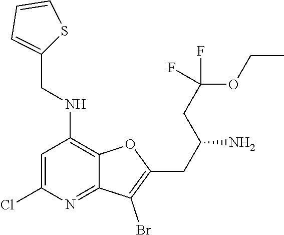 [SMILES: CCOC(F)(F)C[C@H](N)Cc1oc2c(NCc3cccs3)cc(Cl)nc2c1Br] | 2-[(R)-2-amino-4-ethoxy-4,4- difluorobutyl]-3-bromo-5-chloro-7- thenylamino-1-oxa-4-azaindene |
| 213 | 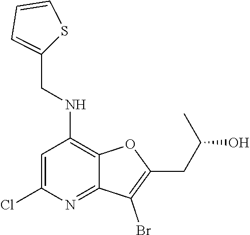 [SMILES: C[C@H](O)Cc1oc2c(NCc3cccs3)cc(Cl)nc2c1Br] | (S)-1-(3-bromo-5-chloro-7-thenylamino- 1-oxa-4-aza-2-indenyl)-2-propanol |
| 4216 | 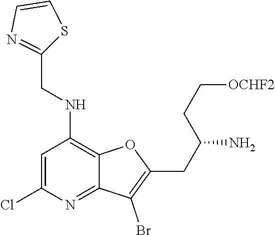 [SMILES: N[C@@H](CCOC(F)F)Cc1oc2c(NCc3nccs3)cc(Cl)nc2c1Br] | 2-[(S)-2-amino-4-difluoromethoxybutyl]- 3-bromo-5-chloro-7-{[(1,3-thiazol-2- yl)methyl]amino}-1-oxa-4-azaindene |
| 217 | 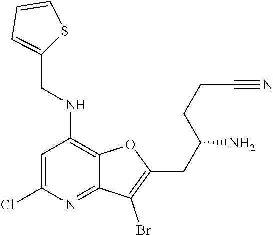 [SMILES: N#CCC[C@H](N)Cc1oc2c(NCc3cccs3)cc(Cl)nc2c1Br] | (S)-4-amino-5-(3-bromo-5-chloro-7- thenylamino-1-oxa-4-aza-2- indenyl)valeronitrile |
| 218 | 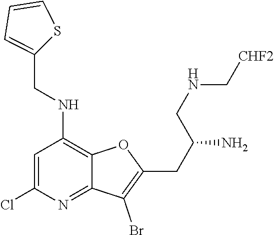 [SMILES: N[C@@H](CNCC(F)F)Cc1oc2c(NCc3cccs3)cc(Cl)nc2c1Br] | (R)-2-amino-3-(3-bromo-5-chloro-7- thenylamino-1-oxa-4-aza-2-indenyl)-1- (2,2-difluoroethylamino)propane |
| 219 | 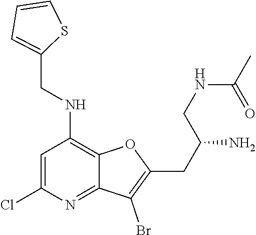 [SMILES: CC(=O)NC[C@H](N)Cc1oc2c(NCc3cccs3)cc(Cl)nc2c1Br] | N-[(R)-2-amino-3-(3-bromo-5-chloro-7- thenylamino-1-oxa-4-aza-2- indenyl)propyl]acetamide |
| 220 | 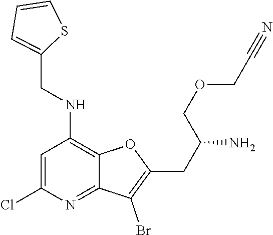 [SMILES: N#CCOC[C@H](N)Cc1oc2c(NCc3cccs3)cc(Cl)nc2c1Br] | [(R)-2-amino-3-(3-bromo-5-chloro-7- thenylamino-1-oxa-4-aza-2- indenyl)propoxy]acetonitrile |
| 221 | 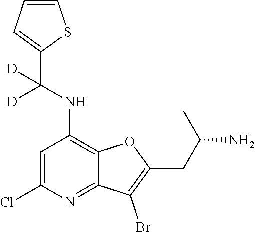 [SMILES: [2H]C([2H])(Nc1cc(Cl)nc2c(Br)c(C[C@H](C)N)oc12)c1cccs1] | 2-[(S)-2-aminopropyl]-3-bromo-5-chloro- 7-{[(2-thienyl)(²H₂)methyl]amino}-1-oxa- 4-azaindene |
| 222 | 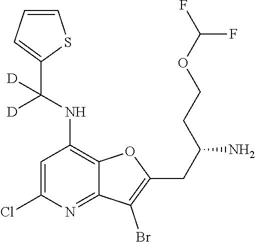 [SMILES: [2H]C([2H])(Nc1cc(Cl)nc2c(Br)c(C[C@@H](N)CCOC(F)F)oc12)c1cccs1] | 2-[(S)-2-amino-4-difluoromethoxybutyl]- 3-bromo-5-chloro-7-{[(2- thienyl)(²H₂)methyl]amino}-1-oxa-4- azaindene |
| 223 | 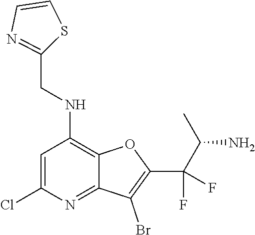 [SMILES: C[C@H](N)C(F)(F)c1oc2c(NCc3nccs3)cc(Cl)nc2c1Br] | 2-[(S)-2-amino-1,1-difluoropropyl]-3- bromo-5-chloro-7-{[(1,3-thiazol-2- yl)methyl]amino}-1-oxa-4-azaindene |
| 224 | 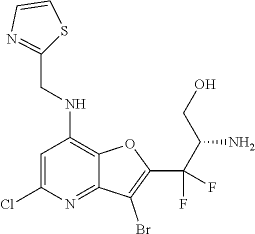 [SMILES: N[C@@H](CO)C(F)(F)c1oc2c(NCc3nccs3)cc(Cl)nc2c1Br] | (S)-2-amino-3-(3-bromo-5-chloro-7-{[(1,3- thiazol-2-yl)methyl]amino}-1-oxa-4-aza-2- indenyl)-3,3-difluoro-1-propanol |
| 225 | 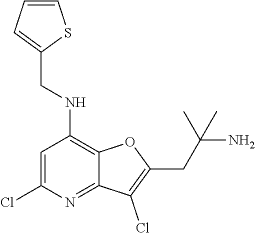 [SMILES: CC(C)(N)Cc1oc2c(NCc3cccs3)cc(Cl)nc2c1Cl] | 2-(2-amino-2-methylpropyl)-3,5-dichloro- 7-thenylamino-1-oxa-4-azaindene |
| 226 | 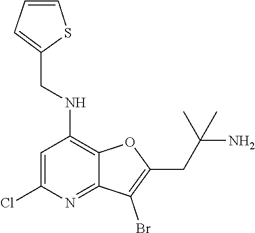 [SMILES: CC(C)(N)Cc1oc2c(NCc3cccs3)cc(Cl)nc2c1Br] | 2-(2-amino-2-methylpropyl)-3-bromo-5- chloro-7-thenylamino-1-oxa-4-azaindene |
| 227 | 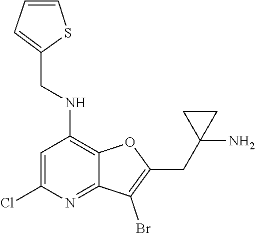 [SMILES: NC1(Cc2oc3c(NCc4cccs4)cc(Cl)nc3c2Br)CC1] | 2-[(1-aminocyclopropyl)methyl]-3-bromo- 5-chloro-7-thenylamino-1-oxa-4- azaindene |
| 228 | 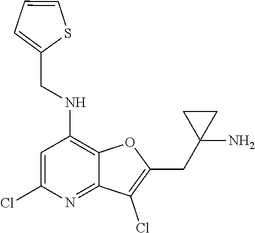 [SMILES: NC1(Cc2oc3c(NCc4cccs4)cc(Cl)nc3c2Cl)CC1] | 2-[(1-aminocyclopropyl)methyl]-3,5- dichloro-7-thenylamino-1-oxa-4- azaindene |
| 229 | 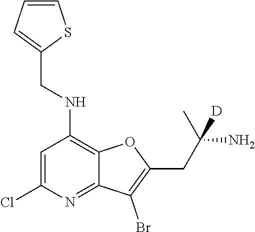 [SMILES: [2H][C@@](C)(N)Cc1oc2c(NCc3cccs3)cc(Cl)nc2c1Br] | 2-[(S)-2-amino(2-2H)propyl]-3-bromo-5- chloro-7-thenylamino-1-oxa-4-azaindene |
| 230 | 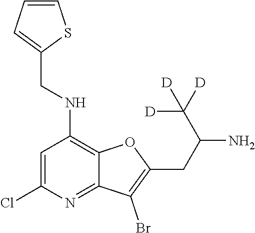 [SMILES: [2H]C([2H])([2H])C(N)Cc1oc2c(NCc3cccs3)cc(Cl)nc2c1Br] | 2-[2-amino(3,3,3-²H₃)propyl]-3-bromo-5- chloro-7-thenylamino-1-oxa-4-azaindene |
| 231 | 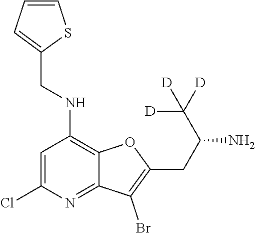 [SMILES: [2H]C([2H])([2H])[C@H](N)Cc1oc2c(NCc3cccs3)cc(Cl)nc2c1Br] | 2-[(S)-2-amino(3,3,3-2H3)propyl]-3- bromo-5-chloro-7-thenylamino-1-oxa-4- azaindene |
| and | | |
| 232 | 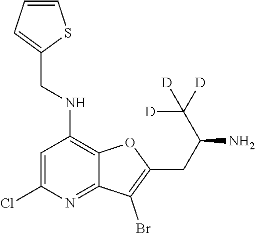 [SMILES: [2H]C([2H])([2H])[C@@H](N)Cc1oc2c(NCc3cccs3)cc(Cl)nc2c1Br] | 2-[(R)-2-amino(3,3,3-²H₃)propyl]-3- bromo-5-chloro-7-thenylamino-1-oxa-4- azaindene |
| 233 | 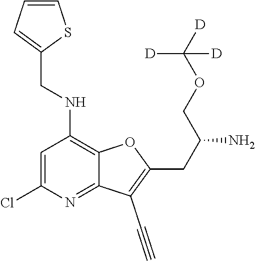 [SMILES: [2H]C([2H])([2H])OC[C@H](N)Cc1oc2c(NCc3cccs3)cc(Cl)nc2c1C#C] | 2-[(R)-2-amino-3-(2H3)methoxypropyl]-5- chloro-3-ethynyl-7-thenylamino-1-oxa-4- azaindene |
| 234 | 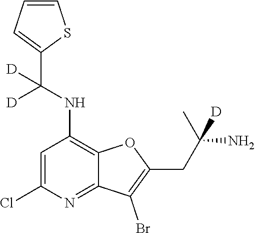 [SMILES: [2H]C([2H])(Nc1cc(Cl)nc2c(Br)c(C[C@]([2H])(C)N)oc12)c1cccs1] | 2-[(S)-2-amino(2-2H)propyl]-3-bromo-5- chloro-7-{[(2-thienyl)(²H₂)methyl]amino}- 1-oxa-4-azaindene |
| 235 | 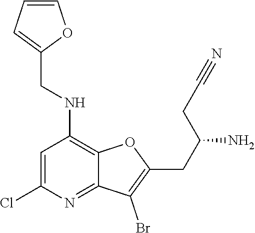 [SMILES: N#CC[C@H](N)Cc1oc2c(NCc3ccco3)cc(Cl)nc2c1Br] | (S)-3-amino-4-[3-bromo-5-chloro-7- (furfurylamino)-1-oxa-4-aza-2- indenyl]butyronitrile |
| 236 | 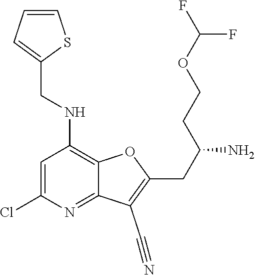 [SMILES: N#Cc1c(C[C@@H](N)CCOC(F)F)oc2c(NCc3cccs3)cc(Cl)nc12] | 2-[(S)-2-amino-4-difluoromethoxybutyl]- 5-chloro-7-thenylamino-1-oxa-4-aza-3- indenecarbonitrile |
| 237 | 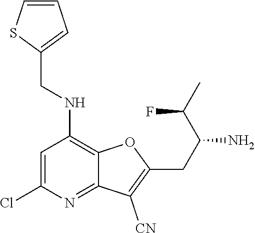 [SMILES: C[C@H](F)[C@H](N)Cc1oc2c(NCc3cccs3)cc(Cl)nc2c1C#N] | 2-[(2R,3S)-2-amino-3-fluorobutyl]-5- chloro-7-thenylamino-1-oxa-4-aza-3- indenecarbonitrile |
| 238 | 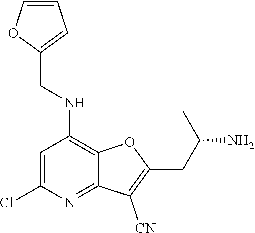 [SMILES: C[C@H](N)Cc1oc2c(NCc3ccco3)cc(Cl)nc2c1C#N] | 2-[(S)-2-aminopropyl]-5-chloro-7- (furfurylamino)-1-oxa-4-aza-3- indenecarbonitrile |
| 239 | 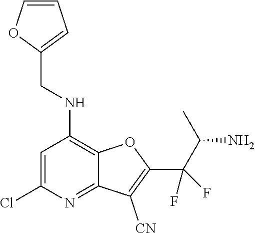 [SMILES: C[C@H](N)C(F)(F)c1oc2c(NCc3ccco3)cc(Cl)nc2c1C#N] | 2-[(S)-2-amino-1,1-difluoropropyl]-3,5- dichloro-7-(furfurylamino)-1-oxa-4- azaindene |
| 240 | 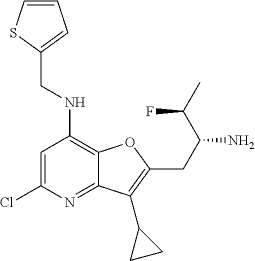 [SMILES: C[C@H](F)[C@H](N)Cc1oc2c(NCc3cccs3)cc(Cl)nc2c1C1CC1] | 2-[(2R,3S)-2-amino-3-fluorobutyl]-5- chloro-3-cyclopropyl-7-thenylamino-1- oxa-4-azaindene |
| 241 | 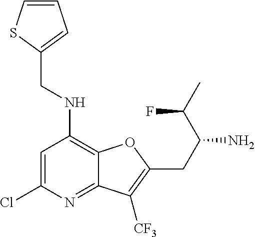 [SMILES: C[C@H](F)[C@H](N)Cc1oc2c(NCc3cccs3)cc(Cl)nc2c1C(F)(F)F] | 2-[(2R,3S)-2-amino-3-fluorobutyl]-5- chloro-7-thenylamino-3-(trifluoromethyl)- 1-oxa-4-azaindene |
| 242 | 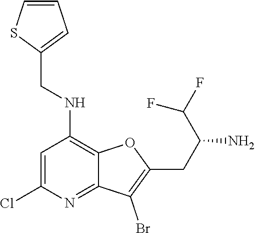 [SMILES: N[C@H](Cc1oc2c(NCc3cccs3)cc(Cl)nc2c1Br)C(F)F] | 2-[(R)-2-amino-3,3-difluoropropyl]-3- bromo-5-chloro-7-thenylamino-1-oxa-4- azaindene |
| 243 | 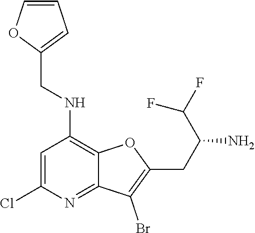 [SMILES: N[C@H](Cc1oc2c(NCc3ccco3)cc(Cl)nc2c1Br)C(F)F] | 2-[(R)-2-amino-3,3-difluoropropyl]-3- bromo-5-chloro-7-(furfurylamino)-1-oxa- 4-azaindene |
| 244 | 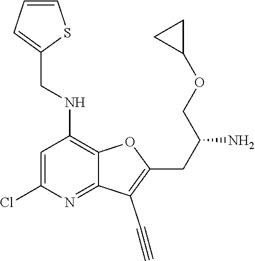 [SMILES: C#Cc1c(C[C@@H](N)COC2CC2)oc2c(NCc3cccs3)cc(Cl)nc12] | 2-[(R)-2-amino-3-cyclopropoxypropyl]-5- chloro-3-ethynyl-7-thenylamino-1-oxa-4- azaindene |
| 245 | 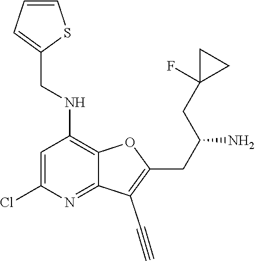 [SMILES: C#Cc1c(C[C@@H](N)CC2(F)CC2)oc2c(NCc3cccs3)cc(Cl)nc12] | 2-[(R)-2-amino-3-(1- fluorocyclopropyl)propyl]-5-chloro-3- ethynyl-7-thenylamino-1-oxa-4-azaindene |
| 246 | 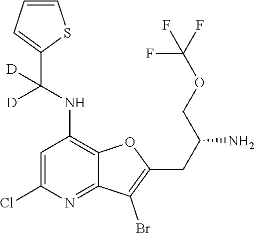 [SMILES: [2H]C([2H])(Nc1cc(Cl)nc2c(Br)c(C[C@@H](N)COC(F)(F)F)oc12)c1cccs1] | 2-[(R)-2-amino-3- trifluoromethoxypropyl]-3-bromo-5- chloro-7-{[(2-thienyl)(²H₂)methyl]amino}- 1-oxa-4-azaindene |
| 247 | 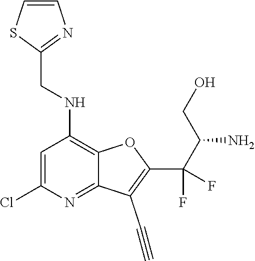 [SMILES: C#Cc1c(C(F)(F)[C@@H](N)CO)oc2c(NCc3nccs3)cc(Cl)nc12] | (S)-2-amino-3-(5-chloro-3-ethynyl-7- {[(1,3-thiazol-2-yl)methyl]amino}-1-oxa-4- aza-2-indenyl)-3,3-difluoro-1-propanol |
| 248 | 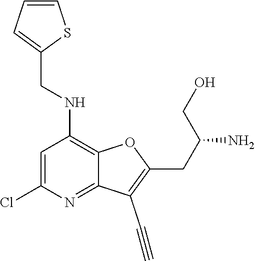 [SMILES: C#Cc1c(C[C@@H](N)CO)oc2c(NCc3cccs3)cc(Cl)nc12] | (R)-2-amino-3-(5-chloro-3-ethynyl-7- thenylamino-1-oxa-4-aza-2-indenyl)-1- propanol |
| 249 | 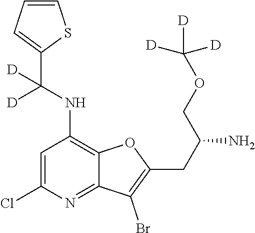 [SMILES: [2H]C([2H])([2H])OC[C@H](N)Cc1oc2c(NC([2H])([2H])c3cccs3)cc(Cl)nc2c1Br] | 2-[(R)-2-amino-3-(²H₃)methoxypropyl]-3- bromo-5-chloro-7-{[(2- thienyl)(²H₂)methyl]amino}-1-oxa-4- azaindene |
| 250 | 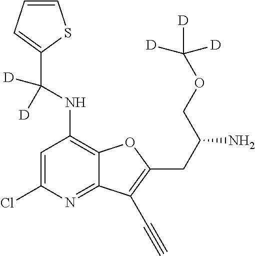 [SMILES: [2H]C([2H])([2H])OC[C@H](N)Cc1oc2c(NC([2H])([2H])c3cccs3)cc(Cl)nc2c1C#C] | 2-[(R)-2-amino-3-(²H₃)methoxypropyl]-5- chloro-3-ethynyl-7-{[(2- thienyl)(²H₂)methyl]amino}-1-oxa-4- azaindene |
| 251 | 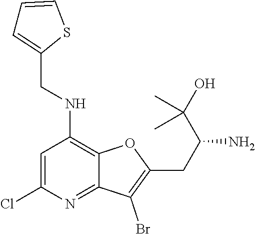 [SMILES: CC(C)(O)[C@H](N)Cc1oc2c(NCc3cccs3)cc(Cl)nc2c1Br] | (R)-3-amino-4-(3-bromo-5-chloro-7- thenylamino-1-oxa-4-aza-2-indenyl)-2- methyl-2-butanol |
| 252 | 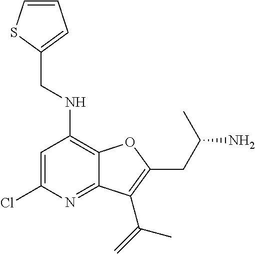 [SMILES: C=C(C)c1c(C[C@H](C)N)oc2c(NCc3cccs3)cc(Cl)nc12] | 2-[(S)-2-aminopropyl]-5-chloro-3- isopropenyl-7-thenylamino-1-oxa-4- azaindene |
| 253 | 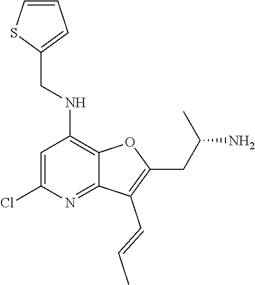 [SMILES: C/C=C/c1c(C[C@H](C)N)oc2c(NCc3cccs3)cc(Cl)nc12] | 3-[(E)-1-propenyl]-2-[(S)-2-aminopropyl]- 5-chloro-7-thenylamino-1-oxa-4- azaindene |
| 254 | 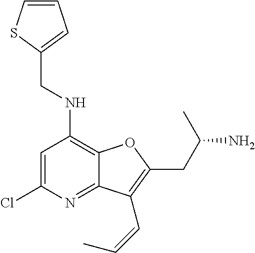 [SMILES: C/C=C\c1c(C[C@H](C)N)oc2c(NCc3cccs3)cc(Cl)nc12] | 2-[(S)-2-aminopropyl]-3-[(Z)-1-propenyl]- 5-chloro-7-thenylamino-1-oxa-4- azaindene |
| 255 | 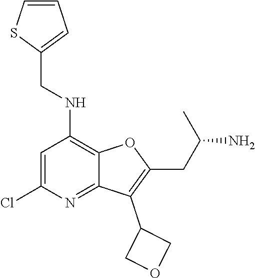 [SMILES: C[C@H](N)Cc1oc2c(NCc3cccs3)cc(Cl)nc2c1C1COC1] | 2-[(S)-2-aminopropyl]-5-chloro-3-(3- oxetanyl)-7-thenylamino-1-oxa-4- azaindene |
| 256 | 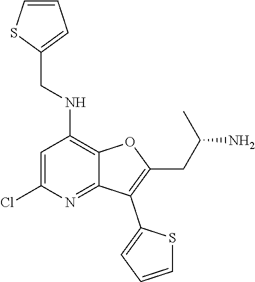 [SMILES: C[C@H](N)Cc1oc2c(NCc3cccs3)cc(Cl)nc2c1-c1cccs1] | 2-[(S)-2-aminopropyl]-5-chloro-7- thenylamino-3-(2-thienyl)-1-oxa-4- azaindene |
| 257 | 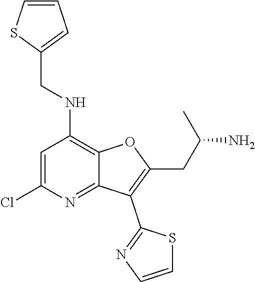 [SMILES: C[C@H](N)Cc1oc2c(NCc3cccs3)cc(Cl)nc2c1-c1nccs1] | 2-[(S)-2-aminopropyl]-5-chloro-7- thenylamino-3-(1,3-thiazol-2-yl)-1-oxa-4- azaindene |
| 258 | 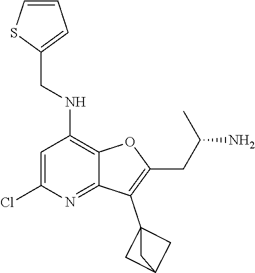 [SMILES: C[C@H](N)Cc1oc2c(NCc3cccs3)cc(Cl)nc2c1C12CC(C1)C2] | 2-[(S)-2-aminopropyl]-3- (bicyclo[1.1.1]pent-1-yl)-5-chloro-7- thenylamino-1-oxa-4-azaindene |
| 259 | 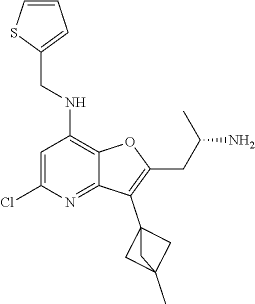 [SMILES: C[C@H](N)Cc1oc2c(NCc3cccs3)cc(Cl)nc2c1C12CC(C)(C1)C2] | 2-[(S)-2-aminopropyl]-5-chloro-3-(3- methylbicyclo[1.1.1]pent-1-yl)-7- thenylamino-1-oxa-4-azaindene |
| 260 | 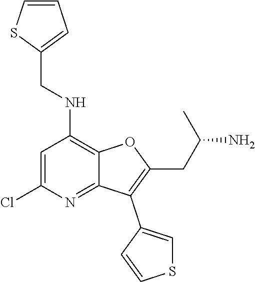 [SMILES: C[C@H](N)Cc1oc2c(NCc3cccs3)cc(Cl)nc2c1-c1ccsc1] | 2-[(S)-2-aminopropyl]-5-chloro-7- thenylamino-3-(3-thienyl)-1-oxa-4- azaindene |
| 261 | 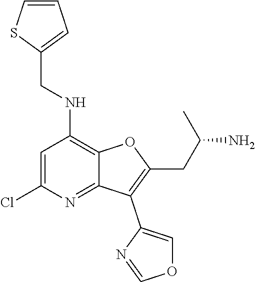 [SMILES: C[C@H](N)Cc1oc2c(NCc3cccs3)cc(Cl)nc2c1-c1cocn1] | 2-[(S)-2-aminopropyl]-5-chloro-3-(1,3- oxazol-4-yl)-7-thenylamino-1-oxa-4- azaindene |
| 262 | 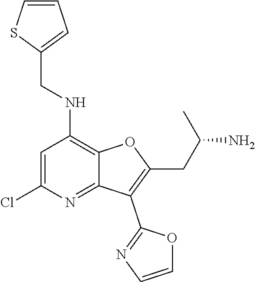 [SMILES: C[C@H](N)Cc1oc2c(NCc3cccs3)cc(Cl)nc2c1-c1ncco1] | 2-[(S)-2-aminopropyl]-5-chloro-3-(1,3- oxazol-2-yl)-7-thenylamino-1-oxa-4- azaindene |
| 263 | 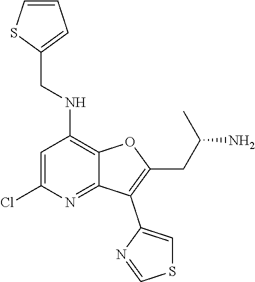 [SMILES: C[C@H](N)Cc1oc2c(NCc3cccs3)cc(Cl)nc2c1-c1cscn1] | 2-[(S)-2-aminopropyl]-5-chloro-7- thenylamino-3-(1,3-thiazol-4-yl)-1-oxa-4- azaindene |
| 264 | 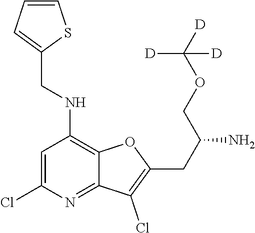 [SMILES: [2H]C([2H])([2H])OC[C@H](N)Cc1oc2c(NCc3cccs3)cc(Cl)nc2c1Cl] | 2-[(R)-2-amino-3-(²H₃)methoxypropyl]- 3,5-dichloro-7-thenylamino-1-oxa-4- azaindene |
| 265 | 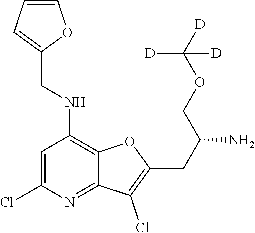 [SMILES: [2H]C([2H])([2H])OC[C@H](N)Cc1oc2c(NCc3ccco3)cc(Cl)nc2c1Cl] | 2-[(R)-2-amino-3-(²H₃)methoxypropyl]- 3,5-dichloro-7-(furfurylamino)-1-oxa-4- azaindene |
| 266 | 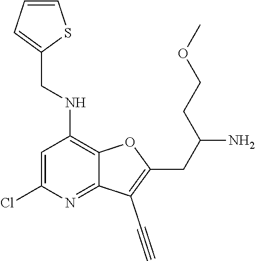 [SMILES: C#Cc1c(CC(N)CCOC)oc2c(NCc3cccs3)cc(Cl)nc12] | 2-[(S)-2-amino-4-methoxybutyl]-5-chloro- 3-ethynyl-7-thenylamino-1-oxa-4- azaindene |
| 267 | 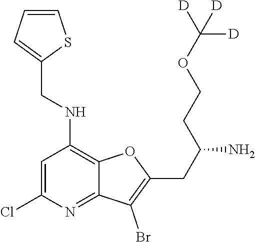 [SMILES: [2H]C([2H])([2H])OCC[C@H](N)Cc1oc2c(NCc3cccs3)cc(Cl)nc2c1Br] | 2-[(S)-2-amino-4-(²H₃)methoxybutyl]-3- bromo-5-chloro-7-thenylamino-1-oxa-4- azaindene |
| 268 | 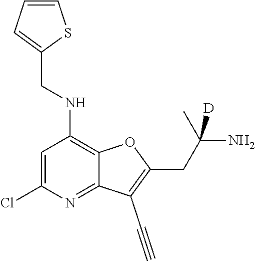 [SMILES: [2H][C@@](C)(N)Cc1oc2c(NCc3cccs3)cc(Cl)nc2c1C#C] | 2-[(S)-2-amino(2-²H)propyl]-5-chloro-3- ethynyl-7-thenylamino-1-oxa-4-azaindene |
| 269 | 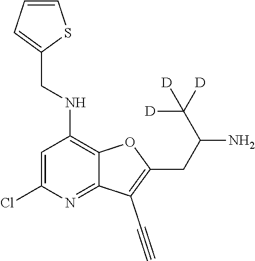 [SMILES: [2H]C([2H])([2H])C(N)Cc1oc2c(NCc3cccs3)cc(Cl)nc2c1C#C] | 2-[2-amino(3,3,3-²H₃)propyl]-5-chloro-3- ethynyl-7-thenylamino-1-oxa-4-azaindene |
| 270 | 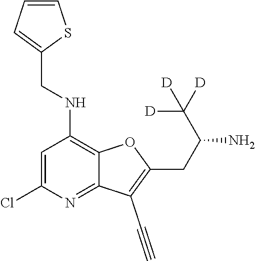 [SMILES: [2H]C([2H])([2H])[C@H](N)Cc1oc2c(NCc3cccs3)cc(Cl)nc2c1C#C] | 2-[(S)-2-amino(3,3,3-²H₃)propyl]-5-chloro- 3-ethynyl-7-thenylamino-1-oxa-4- azaindene |
| and | | |
| 271 | 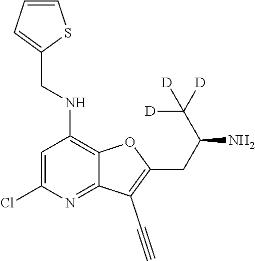 [SMILES: [2H]C([2H])([2H])[C@@H](N)Cc1oc2c(NCc3cccs3)cc(Cl)nc2c1C#C] | 2-[(R)-2-amino(3,3,3-²H₃)propyl]-5- chloro-3-ethynyl-7-thenylamino-1-oxa-4- azaindene |
| 272 | 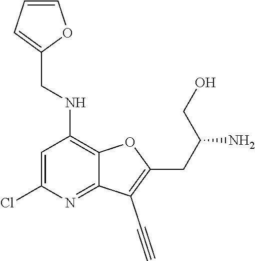 [SMILES: C#Cc1c(C[C@@H](N)CO)oc2c(NCc3ccco3)cc(Cl)nc12] | (R)-2-amino-3-[5-chloro-3-ethynyl-7- (furfurylamino)-1-oxa-4-aza-2-indenyl]-1- propanol |
| 273 | 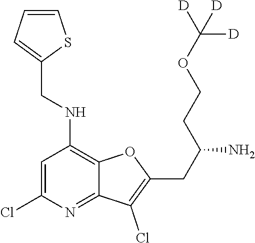 [SMILES: [2H]C([2H])([2H])OCC[C@H](N)Cc1oc2c(NCc3cccs3)cc(Cl)nc2c1Cl] | 2-[(S)-2-amino-4-(²H₃)methoxybutyl]-3,5- dichloro-7-thenylamino-1-oxa-4- azaindene |
| 274 | 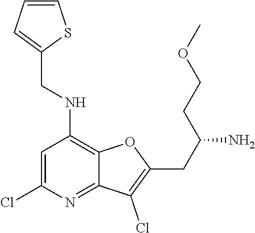 [SMILES: COCC[C@H](N)Cc1oc2c(NCc3cccs3)cc(Cl)nc2c1Cl] | 2-[(S)-2-amino-4-methoxybutyl]-3,5- dichloro-7-thenylamino-1-oxa-4- azaindene |
| 275 | 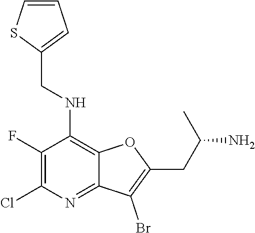 [SMILES: C[C@H](N)Cc1oc2c(NCc3cccs3)c(F)c(Cl)nc2c1Br] | 2-[(S)-2-aminopropyl]-3-bromo-5-chloro- 6-fluoro-7-thenylamino-1-oxa-4- azaindene |
| 276 | 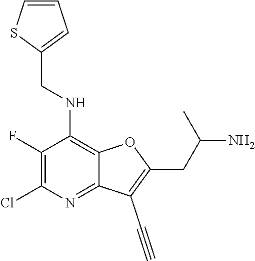 [SMILES: C#Cc1c(CC(C)N)oc2c(NCc3cccs3)c(F)c(Cl)nc12] | 2-[(S)-2-aminopropyl]-5-chloro-3-ethynyl- 6-fluoro-7-thenylamino-1-oxa-4- azaindene |
| 277 | 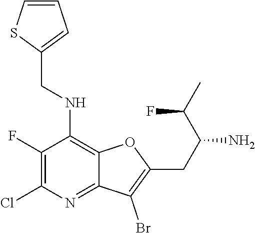 [SMILES: C[C@H](F)[C@H](N)Cc1oc2c(NCc3cccs3)c(F)c(Cl)nc2c1Br] | 2-[(2R,3S)-2-amino-3-fluorobutyl]-3- bromo-5-chloro-6-fluoro-7-thenylamino- 1-oxa-4-azaindene |
| 278 | 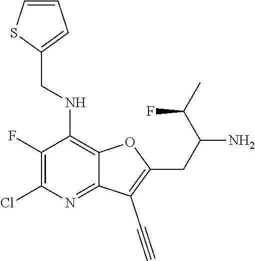 [SMILES: C#Cc1c(CC(N)[C@H](C)F)oc2c(NCc3cccs3)c(F)c(Cl)nc12] | 2-[(2R,3S)-2-amino-3-fluorobutyl]-5- chloro-3-ethynyl-6-fluoro-7-thenylamino- 1-oxa-4-azaindene |
| 279 | 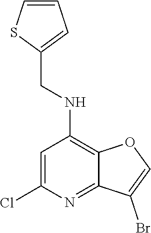 [SMILES: Clc1cc(NCc2cccs2)c2occ(Br)c2n1] | (thenyl)(3-bromo-5-chloro-1-oxa-4-aza-7- indenyl)amine |
| 280 | 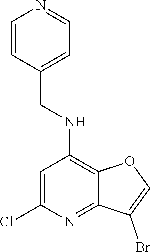 [SMILES: Clc1cc(NCc2ccncc2)c2occ(Br)c2n1] | [(4-pyridyl)methyl](3-bromo-5-chloro-1- oxa-4-aza-7-indenyl)amine |
| 281 | 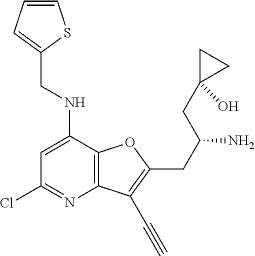 | 1-[(S)-2-amino-3-(5-chloro-3-ethynyl-7- thenylamino-1-oxa-4-aza-2- indenyl)propyl]cyclopropanol |
| 282 | 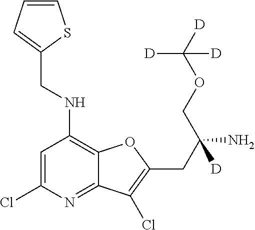 [SMILES: [2H]C([2H])([2H])OC[C@]([2H])(N)Cc1oc2c(NCc3cccs3)cc(Cl)nc2c1Cl] | 2-[(R)-2-amino-3-(²H₃)methoxy(2- 2H)propyl]-3,5-dichloro-7-thenylamino-1- oxa-4-azaindene |
| 283 | 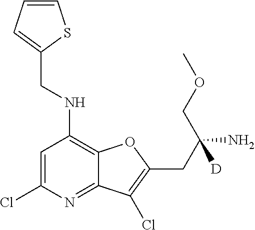 [SMILES: [2H][C@](N)(COC)Cc1oc2c(NCc3cccs3)cc(Cl)nc2c1Cl] | 2-[(R)-2-amino-3-methoxy(2-²H)propyl]- 3,5-dichloro-7-thenylamino-1-oxa-4- azaindene |
| 284 | 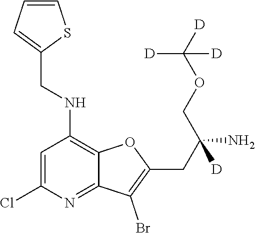 [SMILES: [2H]C([2H])([2H])OC[C@]([2H])(N)Cc1oc2c(NCc3cccs3)cc(Cl)nc2c1Br] | 2-[(R)-2-amino-3-(²H₃)methoxy(2- 2H)propyl]-3-bromo-5-chloro-7- thenylamino-1-oxa-4-azaindene |
| 285 | 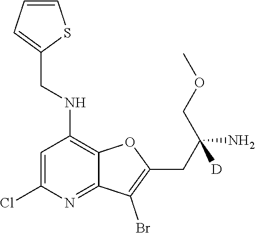 [SMILES: [2H][C@](N)(COC)Cc1oc2c(NCc3cccs3)cc(Cl)nc2c1Br] | 2-[(R)-2-amino-3-methoxy(2-²H)propyl]-3- bromo-5-chloro-7-thenylamino-1-oxa-4- azaindene |
| 286 | 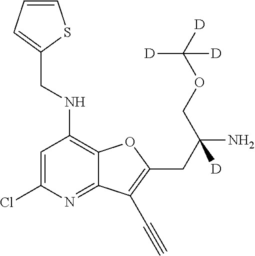 [SMILES: [2H]C([2H])([2H])OC[C@]([2H])(N)Cc1oc2c(NCc3cccs3)cc(Cl)nc2c1C#C] | 2-[(R)-2-amino-3-(²H₃)methoxy(2- 2H)propyl]-5-chloro-3-ethynyl-7- thenylamino-1-oxa-4-azaindene |
| 287 | 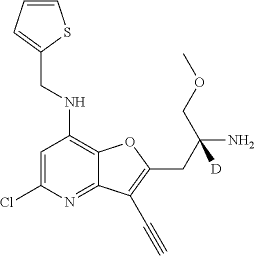 [SMILES: [2H][C@](N)(COC)Cc1oc2c(NCc3cccs3)cc(Cl)nc2c1C#C] | 2-[(R)-2-amino-3-methoxy(2-²H)propyl]-5- chloro-3-ethynyl-7-thenylamino-1-oxa-4- azaindene |
| 288 | 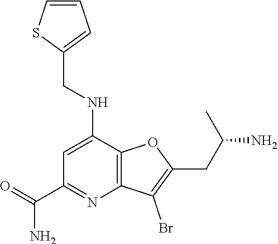 [SMILES: C[C@H](N)Cc1oc2c(NCc3cccs3)cc(C(N)=O)nc2c1Br] | 2-[(S)-2-aminopropyl]-3-bromo-7- thenylamino-1-oxa-4-aza-5- indenecarboxamide |
| 289 | 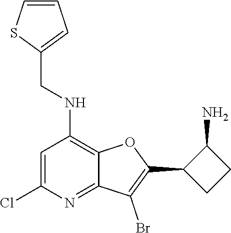 [SMILES: N[C@H]1CC[C@H]1c1oc2c(NCc3cccs3)cc(Cl)nc2c1Br] | 2-[(1R,2S)-2-aminocyclobutyl]-3-bromo-5- chloro-7-thenylamino-1-oxa-4-azaindene |
| 290 | 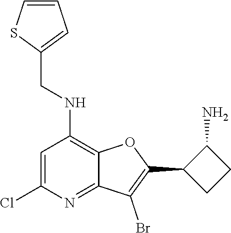 [SMILES: N[C@@H]1CC[C@H]1c1oc2c(NCc3cccs3)cc(Cl)nc2c1Br] | 2-[(1R,2R)-2-aminocyclobutyl]-3-bromo- 5-chloro-7-thenylamino-1-oxa-4- azaindene |
| and | | |
| 291 | 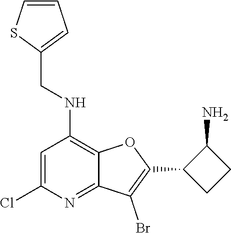 [SMILES: N[C@H]1CC[C@@H]1c1oc2c(NCc3cccs3)cc(Cl)nc2c1Br] | 2-[(1S,2S)-2-aminocyclobutyl]-3-bromo-5- chloro-7-thenylamino-1-oxa-4-azaindene |
| 292 | 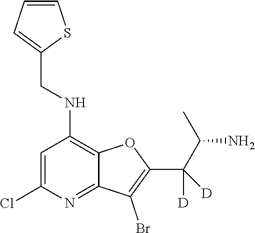 [SMILES: [2H]C([2H])(c1oc2c(NCc3cccs3)cc(Cl)nc2c1Br)[C@H](C)N] | 2-[(S)-2-amino(1,1-²H₂)propyl]-3-bromo- 5-chloro-7-thenylamino-1-oxa-4- azaindene |
| 293 | 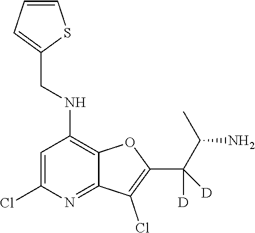 [SMILES: [2H]C([2H])(c1oc2c(NCc3cccs3)cc(Cl)nc2c1Cl)[C@H](C)N] | 2-[(S)-2-amino(1,1-²H₂)propyl]-3,5- dichloro-7-thenylamino-1-oxa-4- azaindene |
| 294 | 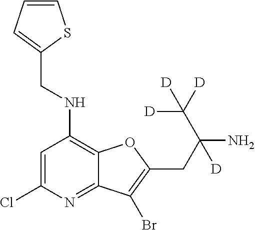 [SMILES: [2H]C([2H])([2H])C([2H])(N)Cc1oc2c(NCc3cccs3)cc(Cl)nc2c1Br] | 2-[2-amino(2,3,3,3-²H₄)propyl]-3-bromo- 5-chloro-7-thenylamino-1-oxa-4- azaindene |
| 295 | 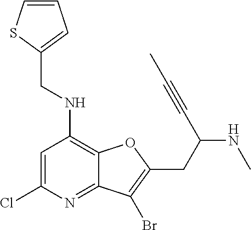 [SMILES: CC#CC(Cc1oc2c(NCc3cccs3)cc(Cl)nc2c1Br)NC] | 2-[(R)-2-(methylamino)-3-pentynyl]-3- bromo-5-chloro-7-thenylamino-1-oxa-4- azaindene |
| 296 | 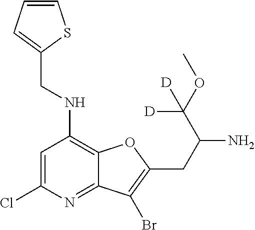 [SMILES: [2H]C([2H])(OC)C(N)Cc1oc2c(NCc3cccs3)cc(Cl)nc2c1Br] | 2-[(R)-2-amino-3-methoxy(3,3- ²H₂)propyl]-3-bromo-5-chloro-7- thenylamino-1-oxa-4-azaindene |
| 297 | 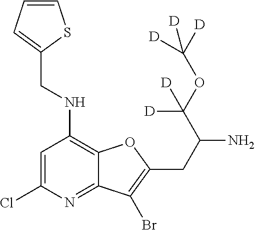 [SMILES: [2H]C([2H])([2H])OC([2H])([2H])C(N)Cc1oc2c(NCc3cccs3)cc(Cl)nc2c1Br] | 2-[(R)-2-amino-3-(²H₃)methoxy(3,3- ²H₂)propyl]-3-bromo-5-chloro-7- thenylamino-1-oxa-4-azaindene |
| 298 | 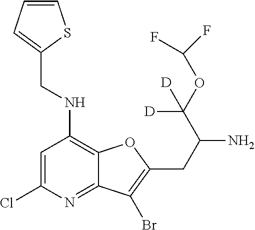 [SMILES: [2H]C([2H])(OC(F)F)C(N)Cc1oc2c(NCc3cccs3)cc(Cl)nc2c1Br] | 2-[(R)-2-amino-3-difluoromethoxy(3,3- ²H₂)propyl]-3-bromo-5-chloro-7- thenylamino-1-oxa-4-azaindene |
| 299 | 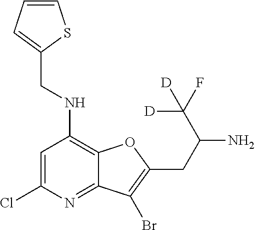 [SMILES: [2H]C([2H])(F)C(N)Cc1oc2c(NCc3cccs3)cc(Cl)nc2c1Br] | 2-[(R)-2-amino-3-fluoro(3,3-²H₂)propyl]-3- bromo-5-chloro-7-thenylamino-1-oxa-4- azaindene |
| 300 | 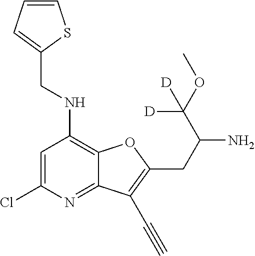 [SMILES: [2H]C([2H])(OC)C(N)Cc1oc2c(NCc3cccs3)cc(Cl)nc2c1C#C] | 2-[(R)-2-amino-3-methoxy(3,3- ²H₂)propyl]-5-chloro-3-ethynyl-7- thenylamino-1-oxa-4-azaindene |
| 301 | 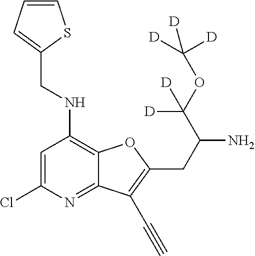 [SMILES: [2H]C([2H])([2H])OC([2H])([2H])C(N)Cc1oc2c(NCc3cccs3)cc(Cl)nc2c1C#C] | 2-[(R)-2-amino-3-(²H₃)methoxy(3,3- ²H₂)propyl]-5-chloro-3-ethynyl-7- thenylamino-1-oxa-4-azaindene |
| 302 | 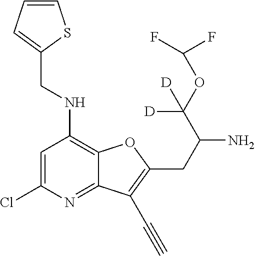 [SMILES: [2H]C([2H])(OC(F)F)C(N)Cc1oc2c(NCc3cccs3)cc(Cl)nc2c1C#C] | 2-[(R)-2-amino-3-difluoromethoxy(3,3- ²H₂)propyl]-5-chloro-3-ethynyl-7- thenylamino-1-oxa-4-azaindene |
| 303 | 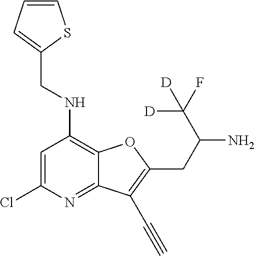 [SMILES: [2H]C([2H])(F)C(N)Cc1oc2c(NCc3cccs3)cc(Cl)nc2c1C#C] | 2-[(R)-2-amino-3-fluoro(3,3-²H₂)propyl]-5- chloro-3-ethynyl-7-thenylamino-1-oxa-4- azaindene |
| 304 | 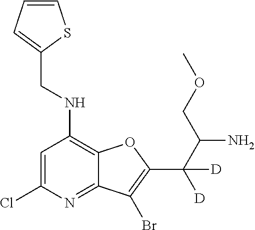 [SMILES: [2H]C([2H])(c1oc2c(NCc3cccs3)cc(Cl)nc2c1Br)C(N)COC] | 2-[(R)-2-amino-3-methoxy(1,1- ²H₂)propyl]-3-bromo-5-chloro-7- thenylamino-1-oxa-4-azaindene |
| 305 | 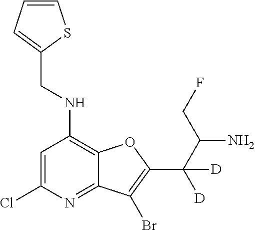 [SMILES: [2H]C([2H])(c1oc2c(NCc3cccs3)cc(Cl)nc2c1Br)C(N)CF] | 2-[(R)-2-amino-3-fluoro(1,1-²H₂)propyl]-3- bromo-5-chloro-7-thenylamino-1-oxa-4- azaindene |
| 306 | 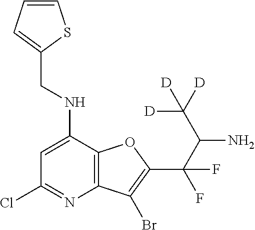 [SMILES: [2H]C([2H])([2H])C(N)C(F)(F)c1oc2c(NCc3cccs3)cc(Cl)nc2c1Br] | 2-[(S)-2-amino-1,1-difluoro(3,3,3- ²H₃)propyl]-3-bromo-5-chloro-7- thenylamino-1-oxa-4-azaindene |
| 307 | 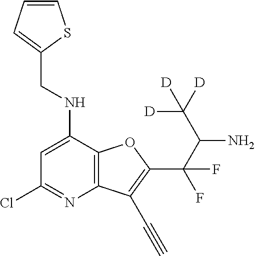 [SMILES: [2H]C([2H])([2H])C(N)C(F)(F)c1oc2c(NCc3cccs3)cc(Cl)nc2c1C#C] | 2-[(S)-2-amino-1,1-difluoro(3,3,3- ²H₃)propyl]-5-chloro-3-ethynyl-7- thenylamino-1-oxa-4-azaindene |
| 308 | 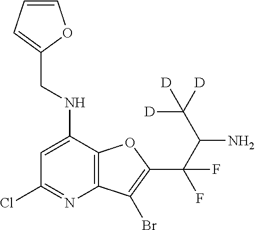 [SMILES: [2H]C([2H])([2H])C(N)C(F)(F)c1oc2c(NCc3ccco3)cc(Cl)nc2c1Br] | 2-[(S)-2-amino-1,1-difluoro(3,3,3- ²H₃)propyl]-3-bromo-5-chloro-7- (furfurylamino)-1-oxa-4-azaindene |
| 309 | 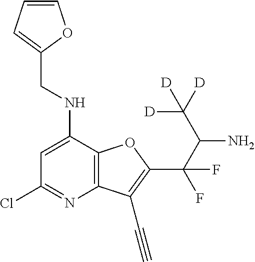 [SMILES: [2H]C([2H])([2H])C(N)C(F)(F)c1oc2c(NCc3ccco3)cc(Cl)nc2c1C#C] | 2-[(S)-2-amino-1,1-difluoro(3,3,3- ²H₃)propyl]-5-chloro-3-ethynyl-7- (furfurylamino)-1-oxa-4-azaindene |
| 310 | 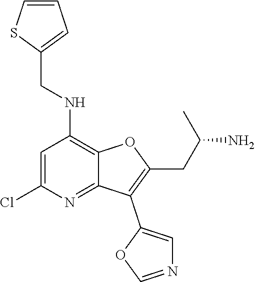 [SMILES: C[C@H](N)Cc1oc2c(NCc3cccs3)cc(Cl)nc2c1-c1cnco1] | 2-[(S)-2-aminopropyl]-5-chloro-3-(1,3- oxazol-5-yl)-7-thenylamino-1-oxa-4- azaindene |
| 311 | 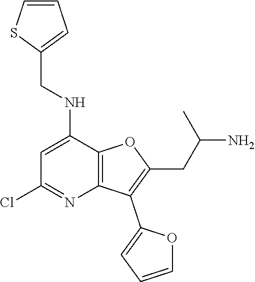 [SMILES: CC(N)Cc1oc2c(NCc3cccs3)cc(Cl)nc2c1-c1ccco1] | 2-[(S)-2-aminopropyl]-5-chloro-3-(2- furyl)-7-thenylamino-1-oxa-4-azaindene |
| 312 | 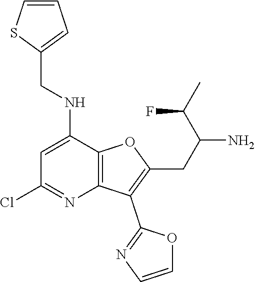 [SMILES: C[C@H](F)C(N)Cc1oc2c(NCc3cccs3)cc(Cl)nc2c1-c1ncco1] | 2-[(2R,3S)-2-amino-3-fluorobutyl]-5- chloro-3-(1,3-oxazol-2-yl)-7-thenylamino- 1-oxa-4-azaindene |
| 313 | 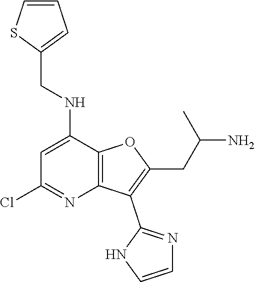 [SMILES: CC(N)Cc1oc2c(NCc3cccs3)cc(Cl)nc2c1-c1ncc[nH]1] | 2-[(S)-2-aminopropyl]-5-chloro-3-(2- imidazolyl)-7-thenylamino-1-oxa-4- azaindene |
| 314 | 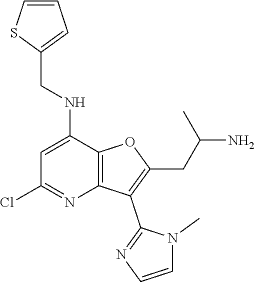 [SMILES: CC(N)Cc1oc2c(NCc3cccs3)cc(Cl)nc2c1-c1nccn1C] | 2-[(S)-2-aminopropyl]-5-chloro-3-(1- methyl-2-imidazolyl)-7-thenylamino-1- oxa-4-azaindene |
| 315 | 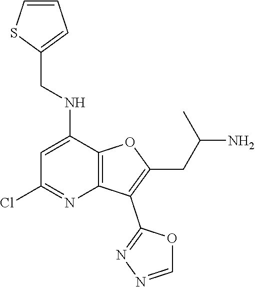 [SMILES: CC(N)Cc1oc2c(NCc3cccs3)cc(Cl)nc2c1-c1nnco1] | 2-[(S)-2-aminopropyl]-5-chloro-3-(1,3,4- oxadiazol-2-yl)-7-thenylamino-1-oxa-4- azaindene |
| 316 | 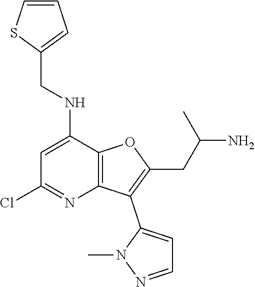 [SMILES: CC(N)Cc1oc2c(NCc3cccs3)cc(Cl)nc2c1-c1ccnn1C] | 2-[(S)-2-aminopropyl]-5-chloro-3-(1- methyl-5-pyrazolyl)-7-thenylamino-1-oxa- 4-azaindene |
| 317 | 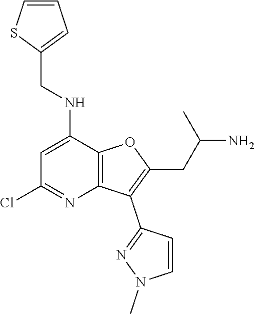 [SMILES: CC(N)Cc1oc2c(NCc3cccs3)cc(Cl)nc2c1-c1ccn(C)n1] | 2-[(S)-2-aminopropyl]-5-chloro-3-(1- methyl-3-pyrazolyl)-7-thenylamino-1-oxa- 4-azaindene |
| 318 | 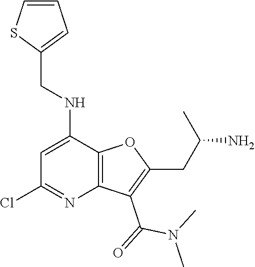 [SMILES: C[C@H](N)Cc1oc2c(NCc3cccs3)cc(Cl)nc2c1C(=O)N(C)C] | N,N-dimethyl-2-[(S)-2-aminopropyl]-5- chloro-7-thenylamino-1-oxa-4-aza-3- indenecarboxamide |
| 319 | 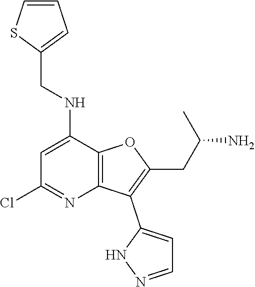 [SMILES: C[C@H](N)Cc1oc2c(NCc3cccs3)cc(Cl)nc2c1-c1ccn[nH]1] | 2-[(S)-2-aminopropyl]-5-chloro-3-(5- pyrazolyl)-7-thenylamino-1-oxa-4- azaindene |
| 320 | 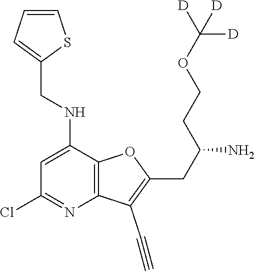 [SMILES: [2H]C([2H])([2H])OCC[C@H](N)Cc1oc2c(NCc3cccs3)cc(Cl)nc2c1C#C] | 2-[(S)-2-amino-4-(²H₃)methoxybutyl]-5- chloro-3-ethynyl-7-thenylamino-1-oxa-4- azaindene |
| 321 | 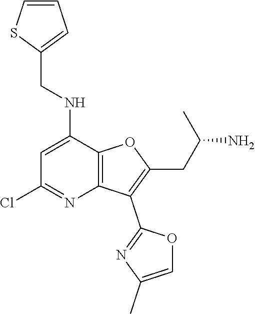 [SMILES: Cc1coc(-c2c(C[C@H](C)N)oc3c(NCc4cccs4)cc(Cl)nc23)n1] | 2-[(S)-2-aminopropyl]-5-chloro-3-(4- methyl-1,3-oxazol-2-yl)-7-thenylamino-1- oxa-4-azaindene |
| 322 | 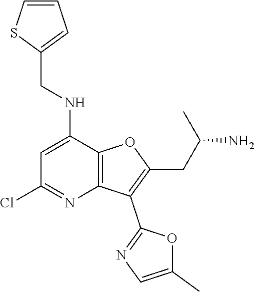 [SMILES: Cc1cnc(-c2c(C[C@H](C)N)oc3c(NCc4cccs4)cc(Cl)nc23)o1] | 2-[(S)-2-aminopropyl]-5-chloro-3-(5- methyl-1,3-oxazol-2-yl)-7-thenylamino-1- oxa-4-azaindene |
| 323 | 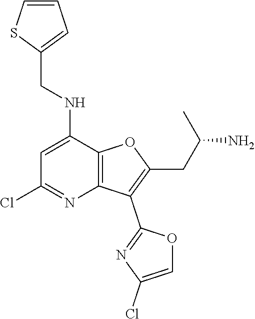 [SMILES: C[C@H](N)Cc1oc2c(NCc3cccs3)cc(Cl)nc2c1-c1nc(Cl)co1] | 2-[(S)-2-aminopropyl]-5-chloro-3-(4- chloro-1,3-oxazol-2-yl)-7-thenylamino-1- oxa-4-azaindene |
| 324 | 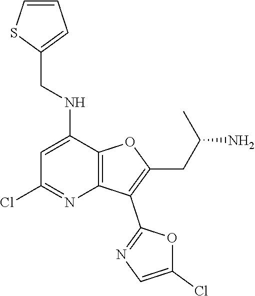 [SMILES: C[C@H](N)Cc1oc2c(NCc3cccs3)cc(Cl)nc2c1-c1ncc(Cl)o1] | 2-[(S)-2-aminopropyl]-5-chloro-3-(5- chloro-1,3-oxazol-2-yl)-7-thenylamino-1- oxa-4-azaindene |
| 325 | 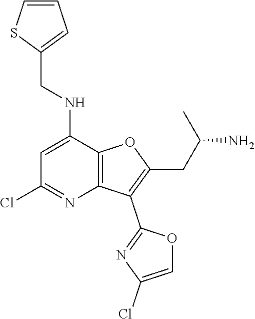 [SMILES: C[C@H](N)Cc1oc2c(NCc3cccs3)cc(Cl)nc2c1-c1nc(Cl)co1] | 2-{2-[(S)-2-aminopropyl]-5-chloro-7- thenylamino-1-oxa-4-aza-3-indenyl}-1,3- oxazole-4-carbonitrile |
| 326 | 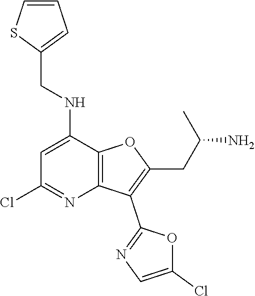 [SMILES: C[C@H](N)Cc1oc2c(NCc3cccs3)cc(Cl)nc2c1-c1ncc(Cl)o1] | 2-{2-[(S)-2-aminopropyl]-5-chloro-7- thenylamino-1-oxa-4-aza-3-indenyl}-1,3- oxazole-5-carbonitrile |
| 327 | 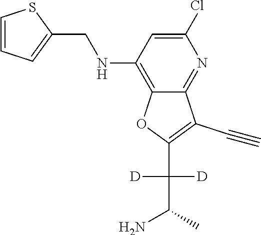 [SMILES: [2H]C([2H])(c1oc2c(NCc3cccs3)cc(Cl)nc2c1C#C)[C@H](C)N] | 2-[(S)-2-amino(1,1-²H₂)propyl]-5-chloro-3- ethynyl-7-thenylamino-1-oxa-4-azaindene |
| 328 | 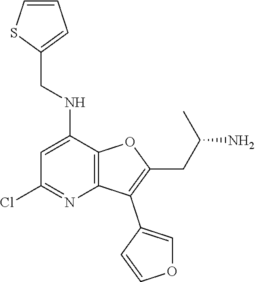 [SMILES: C[C@H](N)Cc1oc2c(NCc3cccs3)cc(Cl)nc2c1-c1ccoc1] | 2-[(S)-2-aminopropyl]-5-chloro-3-(3- furyl)-7-thenylamino-1-oxa-4-azaindene |
| 329 | 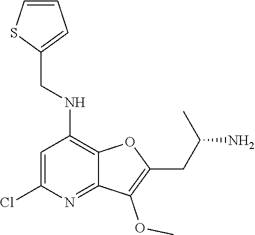 [SMILES: COc1c(C[C@H](C)N)oc2c(NCc3cccs3)cc(Cl)nc12] | 2-[(S)-2-aminopropyl]-5-chloro-3- methoxy-7-thenylamino-1-oxa-4- azaindene |
| 330 | 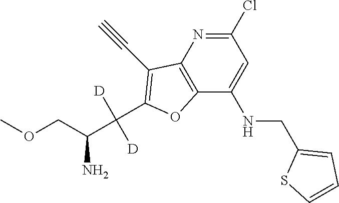 [SMILES: [2H]C([2H])(c1oc2c(NCc3cccs3)cc(Cl)nc2c1C#C)[C@@H](N)COC] | 2-[(R)-2-amino-3-methoxy(1,1- ²H₂)propyl]-5-chloro-3-ethynyl-7- thenylamino-1-oxa-4-azaindene |
| 331 | 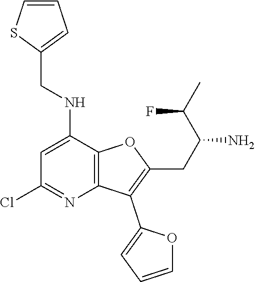 [SMILES: C[C@H](F)[C@H](N)Cc1oc2c(NCc3cccs3)cc(Cl)nc2c1-c1ccco1] | 2-[(2R,3S)-2-amino-3-fluorobutyl]-5- chloro-3-(2-furyl)-7-thenylamino-1-oxa-4- azaindene |
| 332 | 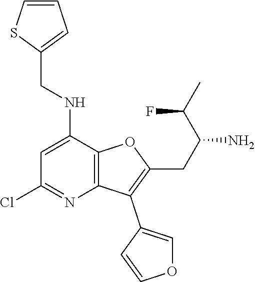 [SMILES: C[C@H](F)[C@H](N)Cc1oc2c(NCc3cccs3)cc(Cl)nc2c1-c1ccoc1] | 2-[(2R,3S)-2-amino-3-fluorobutyl]-5- chloro-3-(3-furyl)-7-thenylamino-1-oxa-4- azaindene |
| 333 | 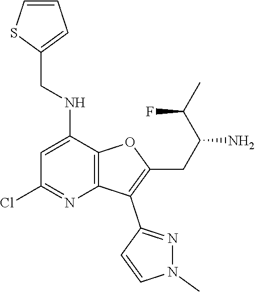 [SMILES: C[C@H](F)[C@H](N)Cc1oc2c(NCc3cccs3)cc(Cl)nc2c1-c1ccn(C)n1] | 2-[(2R,3S)-2-amino-3-fluorobutyl]-5- chloro-3-(1-methyl-3-pyrazolyl)-7- thenylamino-1-oxa-4-azaindene |
| 334 | 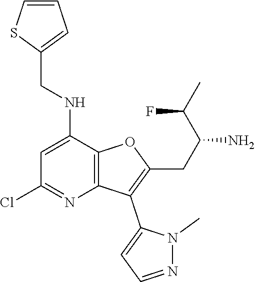 [SMILES: C[C@H](F)[C@H](N)Cc1oc2c(NCc3cccs3)cc(Cl)nc2c1-c1ccnn1C] | 2-[(2R,3S)-2-amino-3-fluorobutyl]-5- chloro-3-(1-methyl-5-pyrazolyl)-7- thenylamino-1-oxa-4-azaindene |
| 335 | 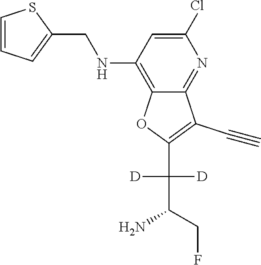 [SMILES: [2H]C([2H])(c1oc2c(NCc3cccs3)cc(Cl)nc2c1C#C)[C@@H](N)CF] | 2-[(R)-2-amino-3-fluoro(1,1-²H₂)propyl]-5- chloro-3-ethynyl-7-thenylamino-1-oxa-4- azaindene |
| 336 | 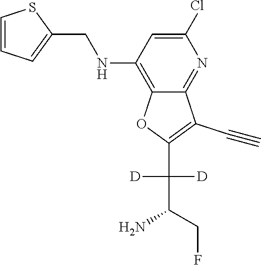 [SMILES: [2H]C([2H])(c1oc2c(NCc3cccs3)cc(Cl)nc2c1C#C)[C@@H](N)CF] | 2-[(R)-2-amino-3-fluoro(1,1-²H₂)propyl]- 3,5-dichloro-7-thenylamino-1-oxa-4- azaindene |
| 337 | 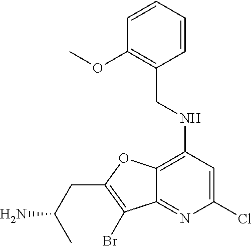 [SMILES: COc1ccccc1CNc1cc(Cl)nc2c(Br)c(C[C@H](C)N)oc12] | 2-[(S)-2-aminopropyl]-3-bromo-5-chloro- 7-{[(o-methoxyphenyl)methyl]amino}-1- oxa-4-azaindene |
| 338 | 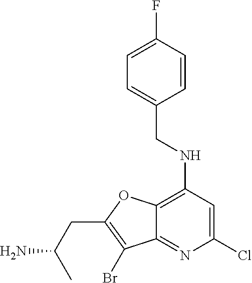 [SMILES: C[C@H](N)Cc1oc2c(NCc3ccc(F)cc3)cc(Cl)nc2c1Br] | 2-[(S)-2-aminopropyl]-3-bromo-5-chloro- 7-{[(p-fluorophenyl)methyl]amino}-1-oxa- 4-azaindene |
| 339 | 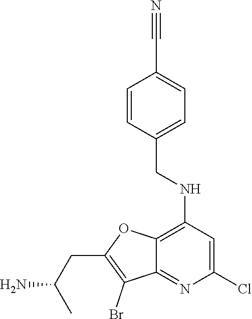 [SMILES: C[C@H](N)Cc1oc2c(NCc3ccc(C#N)cc3)cc(Cl)nc2c1Br] | p-({2-[(S)-2-aminopropyl]-3-bromo-5- chloro-1-oxa-4-aza-7- indenylamino}methyl)benzonitrile |
| 340 | 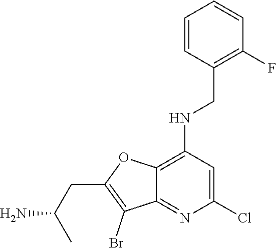 [SMILES: C[C@H](N)Cc1oc2c(NCc3ccccc3F)cc(Cl)nc2c1Br] | 2-[(S)-2-aminopropyl]-3-bromo-5-chloro- 7-{[(o-fluorophenyl)methyl]amino}-1-oxa- 4-azaindene |
| 341 | 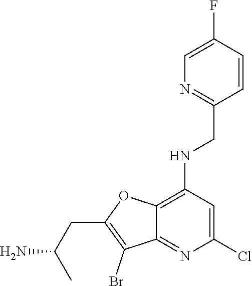 [SMILES: C[C@H](N)Cc1oc2c(NCc3ccc(F)cn3)cc(Cl)nc2c1Br] | 2-[(S)-2-aminopropyl]-3-bromo-5-chloro- 7-{[(5-fluoro-2-pyridyl)methyl]amino}-1- oxa-4-azaindene |
| 342 | 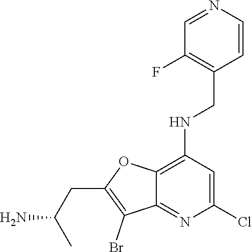 [SMILES: C[C@H](N)Cc1oc2c(NCc3ccncc3F)cc(Cl)nc2c1Br] | 2-[(S)-2-aminopropyl]-3-bromo-5-chloro- 7-{[(3-fluoro-4-pyridyl)methyl]amino}-1- oxa-4-azaindene |
| 343 | 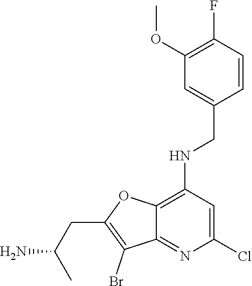 [SMILES: COc1cc(CNc2cc(Cl)nc3c(Br)c(C[C@H](C)N)oc23)ccc1F] | 2-[(S)-2-aminopropyl]-3-bromo-5-chloro- 7-{[(4-fluoro-3- methoxyphenyl)methyl]amino}-1-oxa-4- azaindene |
| 347 | 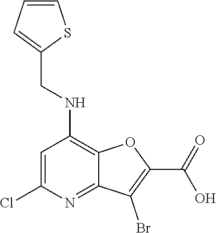 [SMILES: O=C(O)c1oc2c(NCc3cccs3)cc(Cl)nc2c1Br] | 3-bromo-5-chloro-7-thenylamino-1-oxa- 4-aza-2-indenecarboxylic acid |
| 348 | 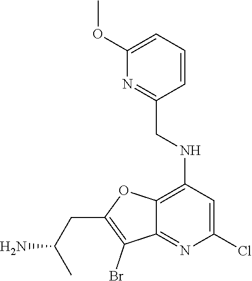 [SMILES: COc1cccc(CNc2cc(Cl)nc3c(Br)c(C[C@H](C)N)oc23)n1] | 2-[(S)-2-aminopropyl]-3-bromo-5-chloro- 7-{[(6-methoxy-2-pyridyl)methyl]amino}- 1-oxa-4-azaindene |
| 349 | 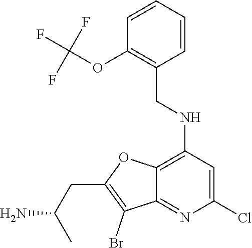 [SMILES: C[C@H](N)Cc1oc2c(NCc3ccccc3OC(F)(F)F)cc(Cl)nc2c1Br] | 2-[(S)-2-aminopropyl]-3-bromo-5-chloro- 7-{[(o- trifluoromethoxyphenyl)methyl]amino}- 1-oxa-4-azaindene |
| 350 | 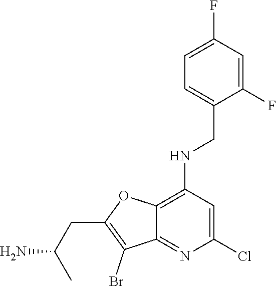 [SMILES: C[C@H](N)Cc1oc2c(NCc3ccc(F)cc3F)cc(Cl)nc2c1Br] | 2-[(S)-2-aminopropyl]-3-bromo-5-chloro- 7-{[(2,4-difluorophenyl)methyl]amino}-1- oxa-4-azaindene |
| 351 | 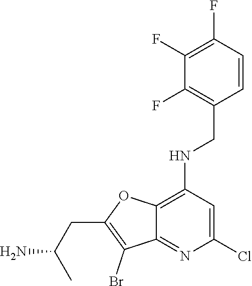 [SMILES: C[C@H](N)Cc1oc2c(NCc3ccc(F)c(F)c3F)cc(Cl)nc2c1Br] | 2-[(S)-2-aminopropyl]-3-bromo-5-chloro- 7-{[(2,3,4-trifluorophenyl)methyl]amino}- 1-oxa-4-azaindene |
| 353 | 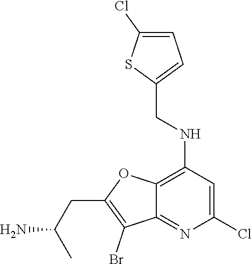 [SMILES: C[C@H](N)Cc1oc2c(NCc3ccc(Cl)s3)cc(Cl)nc2c1Br] | 2-[(S)-2-aminopropyl]-3-bromo-5-chloro- 7-{[(5-chloro-2-thienyl)methyl]amino}-1- oxa-4-azaindene |
| 354 | 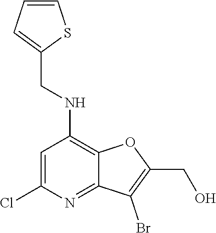 [SMILES: OCc1oc2c(NCc3cccs3)cc(Cl)nc2c1Br] | (3-bromo-5-chloro-7-thenylamino-1-oxa- 4-aza-2-indenyl)methanol |
| 355 | 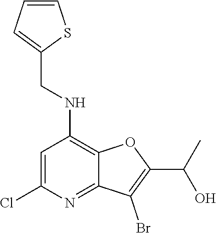 [SMILES: CC(O)c1oc2c(NCc3cccs3)cc(Cl)nc2c1Br] | 1-(3-bromo-5-chloro-7-thenylamino-1- oxa-4-aza-2-indenyl)-1-ethanol |
| 356 | 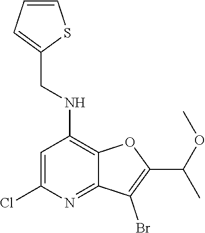 [SMILES: COC(C)c1oc2c(NCc3cccs3)cc(Cl)nc2c1Br] | (thenyl)[3-bromo-5-chloro-2-(1- methoxyethyl)-1-oxa-4-aza-7- indenyl]amine |
| 357 | 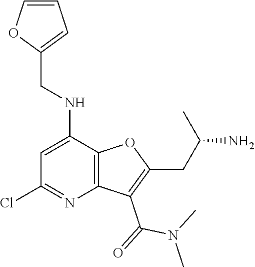 [SMILES: C[C@H](N)Cc1oc2c(NCc3ccco3)cc(Cl)nc2c1C(=O)N(C)C] | N,N-dimethyl-2-[(S)-2-aminopropyl]-5- chloro-7-(furfurylamino)-1-oxa-4-aza-3- indenecarboxamide |
| 358 | 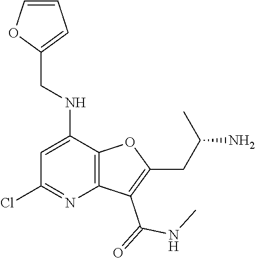 [SMILES: CNC(=O)c1c(C[C@H](C)N)oc2c(NCc3ccco3)cc(Cl)nc12] | N-methyl-2-[(S)-2-aminopropyl]-5-chloro- 7-(furfurylamino)-1-oxa-4-aza-3- indenecarboxamide |
| 359 | 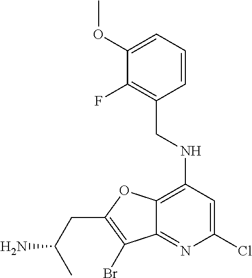 [SMILES: COc1cccc(CNc2cc(Cl)nc3c(Br)c(C[C@H](C)N)oc23)c1F] | 2-[(S)-2-aminopropyl]-3-bromo-5-chloro- 7-{[(2-fluoro-3- methoxyphenyl)methyl]amino}-1-oxa-4- azaindene |
| 360 | 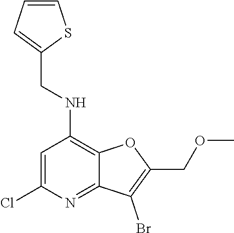 [SMILES: COCc1oc2c(NCc3cccs3)cc(Cl)nc2c1Br] | (thenyl)[3-bromo-5-chloro-2- (methoxymethyl)-1-oxa-4-aza-7- indenyl]amine |
| 361 | 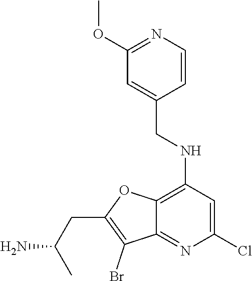 [SMILES: COc1cc(CNc2cc(Cl)nc3c(Br)c(C[C@H](C)N)oc23)ccn1] | 2-[(S)-2-aminopropyl]-3-bromo-5-chloro- 7-{[(2-methoxy-4-pyridyl)methyl]amino}- 1-oxa-4-azaindene |
| 362 | 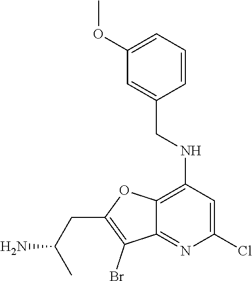 [SMILES: COc1cccc(CNc2cc(Cl)nc3c(Br)c(C[C@H](C)N)oc23)c1] | 2-[(S)-2-aminopropyl]-3-bromo-5-chloro- 7-{[(m-methoxyphenyl)methyl]amino}-1- oxa-4-azaindene |
| 363 | 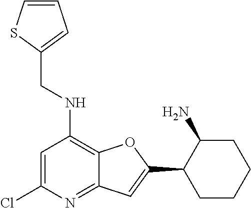 [SMILES: N[C@H]1CCCC[C@H]1c1cc2nc(Cl)cc(NCc3cccs3)c2o1] | Rac-2-[(1R,2S)-2-aminocyclohexyl]-5- chloro-7-thenylamino-1-oxa-4-azaindene |
| | AND | |
| | 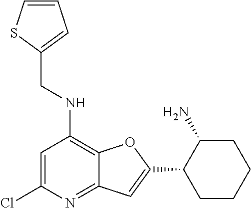 [SMILES: N[C@@H]1CCCC[C@@H]1c1cc2nc(Cl)cc(NCc3cccs3)c2o1] | |
| 364 | 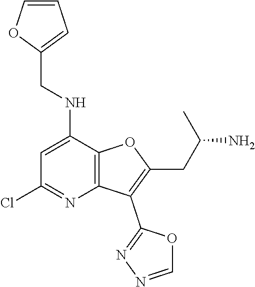 [SMILES: C[C@H](N)Cc1oc2c(NCc3ccco3)cc(Cl)nc2c1-c1nnco1] | 2-[(S)-2-aminopropyl]-5-chloro-7- (furfurylamino)-3-(1,3,4-oxadiazol-2-yl)-1- oxa-4-azaindene |
| 365 | 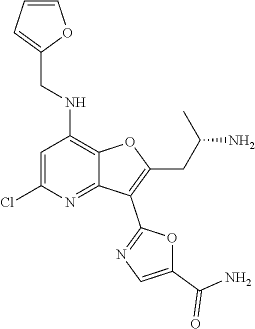 [SMILES: C[C@H](N)Cc1oc2c(NCc3ccco3)cc(Cl)nc2c1-c1ncc(C(N)=O)o1] | 2-{2-[(S)-2-aminopropyl]-5-chloro-7- (furfurylamino)-1-oxa-4-aza-3-indenyl}- 1,3-oxazole-5-carboxamide |
| 366 | 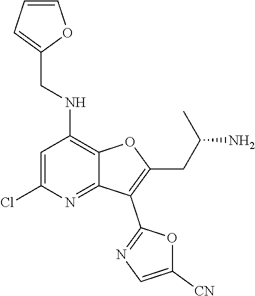 [SMILES: C[C@H](N)Cc1oc2c(NCc3ccco3)cc(Cl)nc2c1-c1ncc(C#N)o1] | 2-{2-[(S)-2-aminopropyl]-5-chloro-7- (furfurylamino)-1-oxa-4-aza-3-indenyl}- 1,3-oxazole-5-carbonitrile |
| 367 | 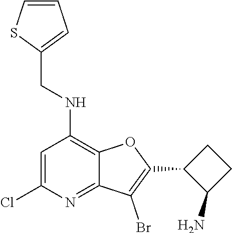 [SMILES: N[C@@H]1CC[C@H]1c1oc2c(NCc3cccs3)cc(Cl)nc2c1Br] | Rac-2-[(1R,2R)-2-aminocyclobutyl]-3- bromo-5-chloro-7-thenylamino-1-oxa-4- azaindene |
| | AND | |
| | 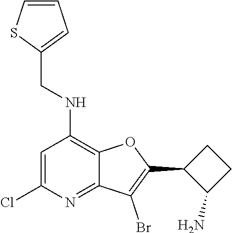 [SMILES: N[C@H]1CC[C@@H]1c1oc2c(NCc3cccs3)cc(Cl)nc2c1Br] | |
| 368 | 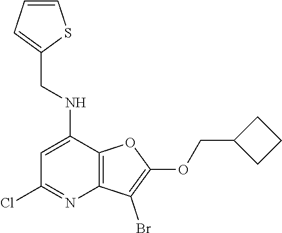 [SMILES: Clc1cc(NCc2cccs2)c2oc(OCC3CCC3)c(Br)c2n1] | (thenyl)[3-bromo-5-chloro-2- (cyclobutylmethoxy)-1-oxa-4-aza-7- indenyl]amine |
| 369 | 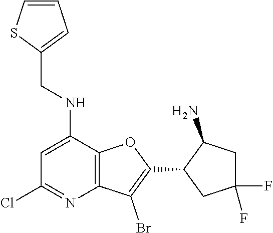 [SMILES: N[C@H]1CC(F)(F)C[C@@H]1c1oc2c(NCc3cccs3)cc(Cl)nc2c1Br] | 2-[(1S,2S)-2-amino-4,4- difluorocyclopentyl]-3-bromo-5-chloro-7- thenylamino-1-oxa-4-azaindene |
| and | | |
| 388 | 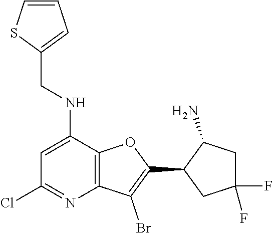 [SMILES: N[C@@H]1CC(F)(F)C[C@H]1c1oc2c(NCc3cccs3)cc(Cl)nc2c1Br] | 2-[(1R,2R)-2-amino-4,4- difluorocyclopentyl]-3-bromo-5-chloro-7- thenylamino-1-oxa-4-azaindene |
| 370 | 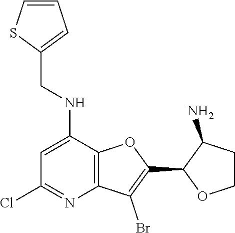 [SMILES: N[C@H]1CCO[C@H]1c1oc2c(NCc3cccs3)cc(Cl)nc2c1Br] | 2-[(2R,3S)-3-aminotetrahydro-2-furyl]-3- bromo-5-chloro-7-thenylamino-1-oxa-4- azaindene |
| 371 | 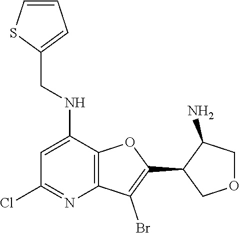 [SMILES: N[C@H]1COC[C@H]1c1oc2c(NCc3cccs3)cc(Cl)nc2c1Br] | 2-[(3S,4R)-4-aminotetrahydro-3-furyl]-3- bromo-5-chloro-7-thenylamino-1-oxa-4- azaindene |
| 372 | 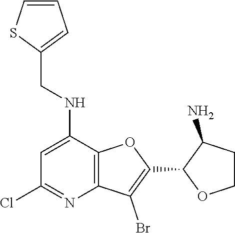 [SMILES: N[C@H]1CCO[C@@H]1c1oc2c(NCc3cccs3)cc(Cl)nc2c1Br] | 2-[(2S,3S)-3-aminotetrahydro-2-furyl]-3- bromo-5-chloro-7-thenylamino-1-oxa-4- azaindene |
| 373 | 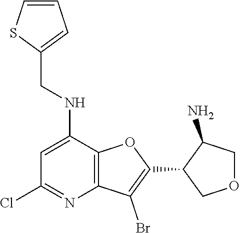 [SMILES: N[C@H]1COC[C@@H]1c1oc2c(NCc3cccs3)cc(Cl)nc2c1Br] | 2-[(3R,4R)-4-aminotetrahydro-3-furyl]-3- bromo-5-chloro-7-thenylamino-1-oxa-4- azaindene |
| 374 | 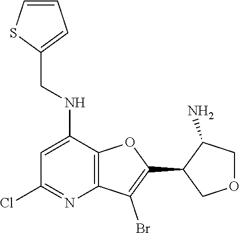 [SMILES: N[C@@H]1COC[C@H]1c1oc2c(NCc3cccs3)cc(Cl)nc2c1Br] | 2-[(3S,4S)-4-aminotetrahydro-3-furyl]-3- bromo-5-chloro-7-thenylamino-1-oxa-4- azaindene |
| 375 | 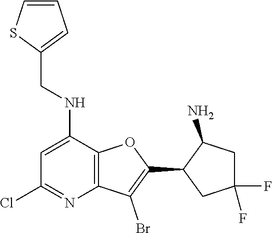 [SMILES: N[C@H]1CC(F)(F)C[C@H]1c1oc2c(NCc3cccs3)cc(Cl)nc2c1Br] | 2-[(1R,2S)-2-amino-4,4- difluorocyclopentyl]-3-bromo-5-chloro-7- thenylamino-1-oxa-4-azaindene |
| 376 | 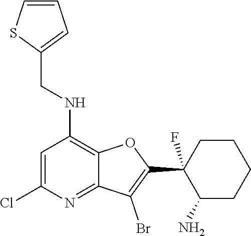 [SMILES: N[C@H]1CCCC[C@]1(F)c1oc2c(NCc3cccs3)cc(Cl)nc2c1Br] | 2-[(1R,2S)-2-amino-1-fluorocyclohexyl]-3- bromo-5-chloro-7-thenylamino-1-oxa-4- azaindene |
| 377 | 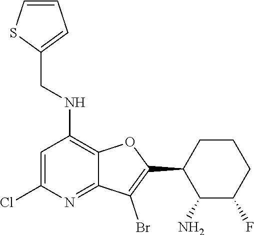 [SMILES: N[C@@H]1[C@@H](c2oc3c(NCc4cccs4)cc(Cl)nc3c2Br)CCC[C@@H]1F] | 2-[(1S,2R,3S)-2-amino-3- fluorocyclohexyl]-3-bromo-5-chloro-7- thenylamino-1-oxa-4-azaindene |
| 378 | 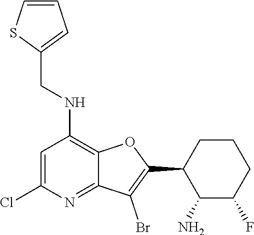 [SMILES: N[C@@H]1[C@@H](c2oc3c(NCc4cccs4)cc(Cl)nc3c2Br)CCC[C@@H]1F] | 2-[(1S,2R,3R)-2-amino-3- fluorocyclohexyl]-3-bromo-5-chloro-7- thenylamino-1-oxa-4-azaindene |
| 379 | 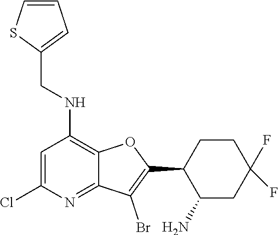 [SMILES: N[C@H]1CC(F)(F)CC[C@@H]1c1oc2c(NCc3cccs3)cc(Cl)nc2c1Br] | 2-[(1S,2S)-2-amino-4,4- difluorocyclohexyl]-3-bromo-5-chloro-7- thenylamino-1-oxa-4-azaindene |
| 380 | 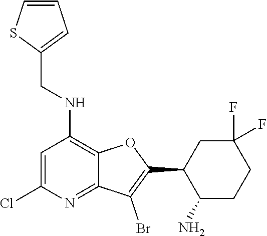 [SMILES: N[C@H]1CCC(F)(F)C[C@@H]1c1oc2c(NCc3cccs3)cc(Cl)nc2c1Br] | 2-[(1S,2S)-2-amino-5,5- difluorocyclohexyl]-3-bromo-5-chloro-7- thenylamino-1-oxa-4-azaindene |
| 381 | 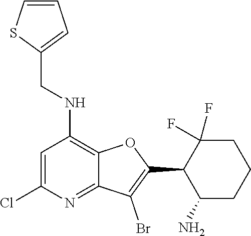 [SMILES: N[C@H]1CCCC(F)(F)[C@@H]1c1oc2c(NCc3cccs3)cc(Cl)nc2c1Br] | 2-[(1S,6S)-6-amino-2,2- difluorocyclohexyl]-3-bromo-5-chloro-7- thenylamino-1-oxa-4-azaindene |
| 382 | 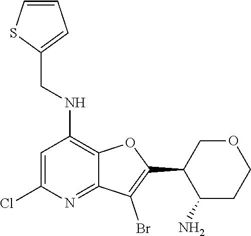 [SMILES: N[C@H]1CCOC[C@@H]1c1oc2c(NCc3cccs3)cc(Cl)nc2c1Br] | 2-[(3R,4S)-4-aminotetrahydro-2H-pyran- 3-yl]-3-bromo-5-chloro-7-thenylamino-1- oxa-4-azaindene |
| 383 | 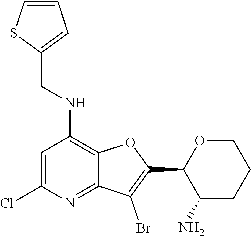 [SMILES: N[C@H]1CCCO[C@@H]1c1oc2c(NCc3cccs3)cc(Cl)nc2c1Br] | 2-[(2S,3S)-3-aminotetrahydro-2H-pyran- 2-yl]-3-bromo-5-chloro-7-thenylamino-1- oxa-4-azaindene |
| 384 | 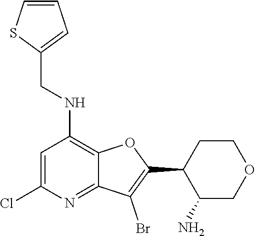 [SMILES: N[C@H]1COCC[C@@H]1c1oc2c(NCc3cccs3)cc(Cl)nc2c1Br] | 2-[(3R,4S)-3-aminotetrahydro-2H-pyran- 4-yl]-3-bromo-5-chloro-7-thenylamino-1- oxa-4-azaindene |
| 385 | 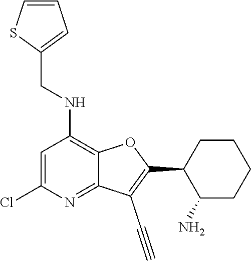 [SMILES: C#Cc1c([C@H]2CCCC[C@@H]2N)oc2c(NCc3cccs3)cc(Cl)nc12] | 2-[(1S,2S)-2-aminocyclohexyl]-5-chloro-3- ethynyl-7-thenylamino-1-oxa-4-azaindene |
| 386 | 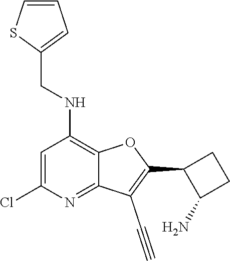 [SMILES: C#Cc1c([C@H]2CC[C@@H]2N)oc2c(NCc3cccs3)cc(Cl)nc12] | 2-[(1S,2S)-2-aminocyclobutyl]-5-chloro-3- ethynyl-7-thenylamino-1-oxa-4-azaindene |
| 387 | 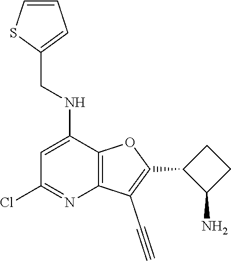 [SMILES: C#Cc1c([C@@H]2CC[C@H]2N)oc2c(NCc3cccs3)cc(Cl)nc12] | 2-[(1R,2R)-2-aminocyclobutyl]-5-chloro-3- ethynyl-7-thenylamino-1-oxa-4-azaindene |
| 389 | 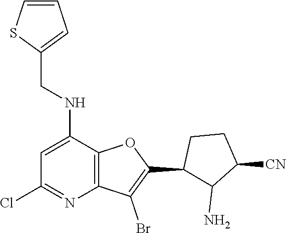 [SMILES: N#C[C@@H]1CC[C@H](c2oc3c(NCc4cccs4)cc(Cl)nc3c2Br)C1N] | (1R,2S,3S)-2-amino-3-(3-bromo-5-chloro- 7-thenylamino-1-oxa-4-aza-2- indenyl) cyclopentanecarbonitrile |
| 390 | 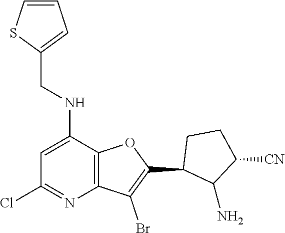 [SMILES: N#C[C@H]1CC[C@H](c2oc3c(NCc4cccs4)cc(Cl)nc3c2Br)C1N] | (1S,2S,3S)-2-amino-3-(3-bromo-5-chloro- 7-thenylamino-1-oxa-4-aza-2- indenyl)cyclopentanecarbonitrile |
| 392 | 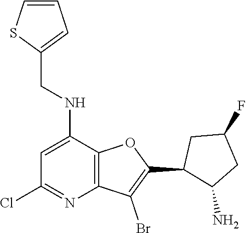 [SMILES: N[C@H]1C[C@H](F)C[C@@H]1c1oc2c(NCc3cccs3)cc(Cl)nc2c1Br] | 2-[(1S,2,4R)-2-amino-4- fluorocyclopentyl]-3-bromo-5-chloro-7- thenylamino-1-oxa-4-azaindene |
| 394 | 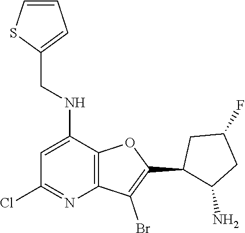 [SMILES: N[C@H]1C[C@@H](F)C[C@@H]1c1oc2c(NCc3cccs3)cc(Cl)nc2c1Br] | 2-[(1S,2S,4S)-2-amino-4- fluorocyclopentyl]-3-bromo-5-chloro-7- thenylamino-1-oxa-4-azaindene |
| 397 | 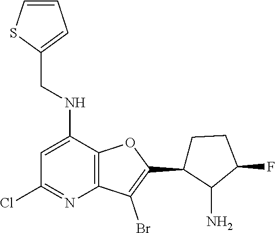 [SMILES: NC1[C@H](F)CC[C@@H]1c1oc2c(NCc3cccs3)cc(Cl)nc2c1Br] | 2-[(1S,2R,3R)-2-amino-3- fluorocyclopentyl]-3-bromo-5-chloro-7- thenylamino-1-oxa-4-azaindene |
| 398 | 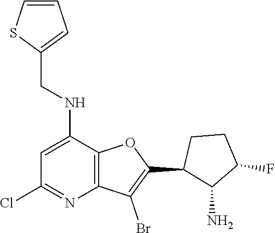 [SMILES: N[C@@H]1[C@@H](c2oc3c(NCc4cccs4)cc(Cl)nc3c2Br)CC[C@@H]1F] | 2-[(1S,2R,3S)-2-amino-3- fluorocyclopentyl]-3-bromo-5-chloro-7- thenylamino-1-oxa-4-azaindene |
| 399 | 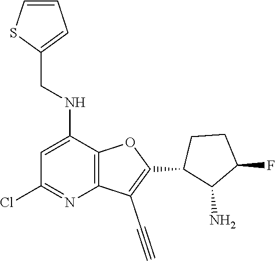 [SMILES: C#Cc1c([C@@H]2CC[C@@H](F)[C@@H]2N)oc2c(NCc3cccs3)cc(Cl)nc12] | 2-[(1R,2R,3R)-2-amino-3- fluorocyclopentyl]-5-chloro-3-ethynyl-7- thenylamino-1-oxa-4-azaindene |
| 400 | 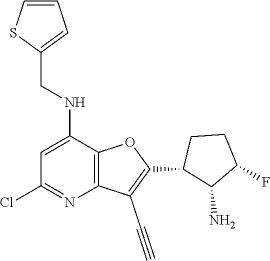 [SMILES: C#Cc1c([C@@H]2CC[C@H](F)[C@@H]2N)oc2c(NCc3cccs3)cc(Cl)nc12] | 2-[(1R,2R,3S)-2-amino-3- fluorocyclopentyl]-5-chloro-3-ethynyl-7- thenylamino-1-oxa-4-azaindene |
| 401 | 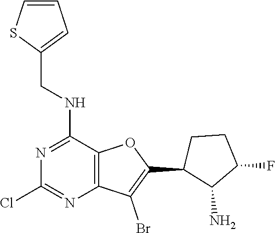 [SMILES: N[C@@H]1[C@@H](c2oc3c(NCc4cccs4)nc(Cl)nc3c2Br)CC[C@@H]1F] | 2-[(1S,2R,3S)-2-amino-3- fluorocyclopentyl]-3-bromo-5-chloro-7- thenylamino-1-oxa-4,6-diazaindene |
| 402 | 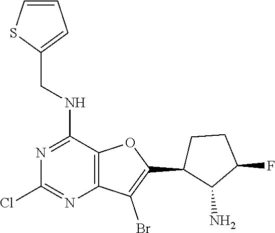 [SMILES: N[C@H]1[C@H](F)CC[C@@H]1c1oc2c(NCc3cccs3)nc(Cl)nc2c1Br] | 2-[(1S,2R,3R)-2-amino-3- fluorocyclopentyl]-3-bromo-5-chloro-7- thenylamino-1-oxa-4,6-diazaindene |
| 403 | 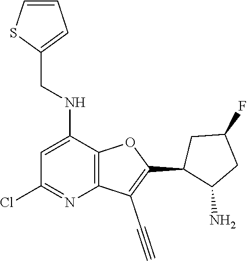 [SMILES: C#Cc1c([C@H]2C[C@@H](F)C[C@@H]2N)oc2c(NCc3cccs3)cc(Cl)nc12] | 2-[(1S,2S,4R)-2-amino-4- fluorocyclopentyl]-5-chloro-3-ethynyl-7- thenylamino-1-oxa-4-azaindene |
| 404 | 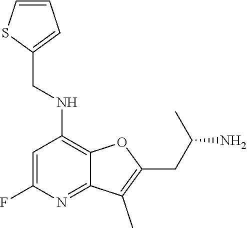 [SMILES: Cc1c(C[C@H](C)N)oc2c(NCc3cccs3)cc(F)nc12] | 2-[(S)-2-aminopropyl]-5-fluoro-3-methyl- 7-thenylamino-1-oxa-4-azaindene |

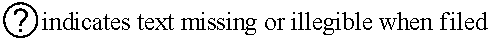

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically.

Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of a SMSM described herein with one or more other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

The compositions described herein can be administered to the subject in a variety of ways, including parenterally, intravenously, intradermally, intramuscularly, colonically, rectally, or intraperitoneally. In some embodiments, the small molecule splicing modulator, or a pharmaceutically acceptable salt thereof is administered by intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a small molecule splicing modulator can be in any suitable form for oral administration, such as liquid, tablets, capsules, or the like. The oral formulations can be further coated or treated to prevent or reduce dissolution in stomach. The compositions of the present disclosure can be administered to a subject using any suitable methods known in the art. Suitable formulations for use in the present disclosure and methods of delivery are generally well known in the art. For example, the small molecule splicing modulators described herein can be formulated as pharmaceutical compositions with a pharmaceutically acceptable diluent, carrier, or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

In some embodiments, the pharmaceutical formulation is in the form of a tablet. In other embodiments, pharmaceutical formulations containing a SMSM described herein are in the form of a capsule. In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a SMSM described herein can be formulated for use as an aerosol, a mist, or a powder. For buccal or sublingual administration, the compositions may take the form of tablets, lozenges, or gels formulated in a conventional manner. In some embodiments, a SMSM described herein can be prepared as transdermal dosage forms. In some embodiments, a SMSM described herein can be formulated into a pharmaceutical composition suitable for intramuscular, subcutaneous, or intravenous injection. In some embodiments, a SMSM described herein can be administered topically and can be formulated into a variety of topically administrable compositions, such as solutions, suspensions, lotions, gels, pastes, medicated sticks, balms, creams, or ointments. In some embodiments, a SMSM described herein can be formulated in rectal compositions such as enemas, rectal gels, rectal foams, rectal aerosols, suppositories, jelly suppositories, or retention enemas.

In some embodiments, disclosed herein is a pharmaceutical composition comprising a compound of the disclosure or a pharmaceutically acceptable salt thereof, and a pharmaceutically acceptable excipient or carrier.

### Splicing Modulation of Target Gene Products

The present disclosure contemplates use of small molecules with favorable drug properties that modulate the activity of splicing of a target RNA. Provided herein are small molecule splicing modulators (SMSMs) that modulate splicing of a polynucleotide. In some embodiments, the SMSMs bind and modulate target RNA. In some embodiments, provided herein is a library of SMSMs that bind and modulate one or more target RNAs. In some embodiments, the target RNA is mRNA. In some embodiments, the target RNA is a noncoding RNA. In some embodiments, the target RNA is a pre-mRNA. In some embodiments, the target RNA is hnRNA. In some embodiments, the small molecules modulate splicing of the target RNA. In some embodiments, a small molecule provided herein modulates splicing at a sequence of the target RNA. In some embodiments, a small molecule provided herein modulates splicing at a cryptic splice site sequence of the target RNA. In some embodiments, a small molecule provided herein modulates splicing at an alternative splice site sequence of the target RNA. In some embodiments, a small molecule provided herein modulates splicing at a native splice site sequence of the target RNA. In some embodiments, a small molecule provided herein binds to a target RNA. In some embodiments, a small molecule provided herein binds to a splicing complex or a component thereof. In some embodiments, a small molecule provided herein binds to a target RNA and a splicing complex or a component thereof. In some embodiments, a small molecule provided herein modulates binding affinity of a splicing complex component to a target RNA such as a pre-mRNA. In some embodiments, a small molecule provided herein modulates binding affinity of a splicing complex component to a target RNA such as a pre-mRNA at a splice site sequence. In some embodiments, a small molecule provided herein modulates binding affinity of a splicing complex component to a target RNA such as a pre-mRNA upstream of a splice site sequence or downstream of a splice site sequence.

Described herein are compounds modifying splicing of gene products, such as Ataxin 3 pre-mRNA for use in the treatment, prevention, and/or delay of progression of diseases or conditions.

In some embodiments, described herein, is a method of treating, preventing, delaying of progress, or ameliorating symptoms of a disease or a condition associated with Ataxin 3 (ATXN3) expression level or activity level in a subject in need thereof, comprising administering a therapeutically effective amount of a small molecule splicing modulator (SMSM), wherein the SMSM binds to a pre-mRNA encoded by ATXN3 and modulates splicing of the ATXN3 pre-mRNA in a cell of the subject to produce a spliced product of the ATXN3 pre-mRNA.

In some embodiments, described herein is a method of treating, preventing, delaying of progress, or ameliorating symptoms of a disease or a condition associated with Ataxin 3 (ATXN3) expression level or activity level in a subject in need thereof, comprising administering a therapeutically effective amount of a compound or salt of Formula (I). In some embodiments, described herein is a method of modulating splicing of a Ataxin3 (ATXN3) pre-mRNA, comprising contacting a compound or salt of Formula (I) to the ATXN3 pre-mRNA with a splice site sequence or cells comprising the ATXN3 pre-mRNA, wherein the compound binds to the ATXN3 pre-mRNA and modulates splicing of the ATXN3 pre-mRNA in a cell of a subject to produce a spliced product of the ATXN3 pre-mRNA. In some embodiments, described herein is use of a compound of Formula (I), or a pharmaceutically acceptable salt thereof, in the manufacture of a medicament for the treatment of a condition or disease associated with Ataxin 3 (ATXN3) expression level or activity level.

In some embodiments, the spliced product of the ATXN3 pre-mRNA undergoes non-sense mediated decay (NMD) and/or nuclear retention. In some embodiments, the nonsense-mediated decay (NMD) and/or nuclear retention of the spliced product of the ATXN3 pre-mRNA is promoted. In some embodiments, the nonsense-mediated decay (NMD) and/or nuclear retention of the spliced product of the ATXN3 pre-mRNA is increased compared to a spliced product of the ATXN3 pre-mRNA produced in the absence of the SMSM.

In some embodiments, described herein is a method of modulating splicing of a Ataxin3 (ATXN3) pre-mRNA, comprising contacting a small molecule splicing modulator (SMSM) to the ATXN3 pre-mRNA with a splice site sequence or cells comprising the ATXN3 pre-mRNA, wherein the SMSM binds to the ATXN3 pre-mRNA and modulates splicing of the ATXN3 pre-mRNA in a cell of a subject to produce a spliced product of the ATXN3 pre-mRNA.

In some embodiments, described herein, is a method of modulating splicing of Ataxin 3 (ATXN3) pre-mRNA, comprising contacting a small molecule splicing modulator (SMSM) to the ATXN3 pre-mRNA with a splice site sequence or cells comprising the ATXN3 pre-mRNA, wherein the SMSM binds to the ATXN3 pre-mRNA and modulates splicing of the ATXN3 pre-mRNA in a cell of a subject to produce a spliced product of the ATXN3 pre-mRNA, wherein the splice site sequence comprises UCCUAU/guaagauucugu.

In some embodiments, described herein, is a method of treating, preventing, delaying of progress, or ameliorating symptoms of a disease or condition associated with Ataxin 3 (ATXN3) expression level or activity level in a subject in need thereof, comprising administering a therapeutically effective amount of a small molecule splicing modulator (SMSM) to the subject, wherein the SMSM binds to a ATXN3 pre-mRNA with a splice site sequence and modulates splicing of the ATXN3 pre-mRNA in a cell of the subject, wherein a spliced product of the ATXN3 pre-mRNA undergoes nonsense-mediated decay (NMD), and wherein the splice site sequence comprises UCCUAU/guaagauucugu.

In some embodiments, the modulating splicing comprises modulating alternative splicing. In some embodiments, the modulating splicing comprises promoting exon skipping. In some embodiments, the modulating splicing comprises promoting exon inclusion. In some embodiments, the modulating splicing comprises modulating nonsense-mediated mRNA decay (NMD). In some embodiments, the modulating NMD comprises promoting NMD. In some embodiments, the modulating splicing comprises modulating nuclear retention of the spliced product of the pre-mRNA. In some embodiments, the modulating intron retention comprises promoting nuclear retention of the spliced product of the pre-mRNA.

In some embodiments, the splice site sequence is a native splice site sequence. In some embodiments, the native splice site is a canonical splice site. In some embodiments, the native splice site is an alternative splice site. In some embodiments, the alternative splice site comprises a 5′ splice site sequence. In some embodiments, the alternative splice site sequence comprises UCCUAU/guaagauucugu. In some embodiments, the SMSM induces splicing at the alternative splice site. In some embodiments, the splicing at the alternative splice site results in a frameshift in a downstream exon in the spliced product. In some embodiments, the downstream exon comprises an in-frame stop codon that is not in frame in the absence of splicing at the alternative splice site. In some embodiments, the in-frame stop codon in the downstream exon is at least 50 or at least 60 base pairs upstream of the 3′ end of the downstream exon. In some embodiments, the in-frame stop codon in the downstream exon is at least 50 or at least 60 base pairs upstream of a final exon-exon junction.

In some embodiments, the splicing of the pre-mRNA at the alternative splice site promotes NMD of the spliced product of the ATXN3 pre-mRNA. In some embodiments, the spliced product comprises an alternative exon. In some embodiments, the SMSM promotes inclusion of the alternative exon in the spliced product. In some embodiments, the alternative exon comprises a poison exon. In some embodiments, the SMSM promotes inclusion of the poison exon in the spliced product. In some embodiments, the poison exon comprises an in-frame stop codon. In some embodiments, the in-frame stop codon is a premature termination codon. In some embodiments, the in-frame stop codon is at least 50 or 60 base pairs upstream of the 3′ end of the poison exon. In some embodiments, the in-frame stop codon is less than 60 base pairs upstream of the 3′ end of the poison exon and wherein the exon immediately downstream of the poison exon is not the last exon in the pre-mRNA. In some embodiments, the sum of (a) the number of base pairs in the exon immediately downstream of the poison exon and (b) the number of base pairs between the premature termination codon in the poison exon and the 3′ end of the poison exon is at least 50 or at least 60.

In some embodiments, the cells comprise primary cells. In some embodiments, the cells comprise disease cells. In some embodiments, the SMSM modulates proliferation or survival of the cells. In some embodiments, the SMSM modulates the expression level of a protein encoded by the spliced product of the pre-mRNA in the cells.

TABLE 2
Exemplary targets for exon skipping

| Gene | ATXN3 |
|---|---|
| Exon Coordinates | Chr14:92093746-92093831 |
| Splicing Event Region | chr14:92093319-92096092 |
| Strand | — |
| Target site | Exon 4 |
| Exon length | 86 |
| SEQ ID NO: | 1 |
| 5′ ss sequence (−6~+12) | UCCUAU/guaagauucugu |
| 5′ ss-U1 duplex structure | -1U-C loop |
| Disease | Spinocerebellar Ataxia Type 3 |

### Methods of Treatment

The compositions and methods described herein can be used for treating a human disease or disorder associated with aberrant splicing, such as aberrant pre-mRNA splicing. The compositions and methods described herein can be used for treating a human disease or disorder by modulating mRNA, such as pre-mRNA. In some embodiments, the compositions and methods described herein can be used for treating a human disease or disorder by modulating splicing of a nucleic acid even when that nucleic acid is not aberrantly spliced in the pathogenesis of the disease or disorder being treated.

In some embodiments, an effective amount in the context of the administration of a SMSM or a pharmaceutically acceptable salt thereof, or composition or medicament thereof refers to an amount of a SMSM or a pharmaceutically acceptable salt thereof to a patient which has a therapeutic effect and/or beneficial effect. In certain specific embodiments, an effective amount in the context of the administration of a SMSM or a pharmaceutically acceptable salt thereof, or composition or medicament thereof to a patient results in one, two or more of the following effects: (i) reduces or ameliorates the severity of a disease; (ii) delays onset of a disease; (iii) inhibits the progression of a disease; (iv) reduces hospitalization of a subject; (v) reduces hospitalization length for a subject; (vi) increases the survival of a subject; (vii) improves the quality of life of a subject; (viii) reduces the number of symptoms associated with a disease; (ix) reduces or ameliorates the severity of a symptom associated with a disease; (x) reduces the duration of a symptom associated with a disease associated; (xi) prevents the recurrence of a symptom associated with a disease; (xii) inhibits the development or onset of a symptom of a disease; and/or (xiii) inhibits of the progression of a symptom associated with a disease. In some embodiments, an effective amount of a SMSM or a pharmaceutically acceptable salt thereof is an amount effective to restore the amount of an RNA transcript of a gene to the amount of the RNA transcript detectable in healthy patients or cells from healthy patients. In other embodiments, an effective amount of a SMSM or a pharmaceutically acceptable salt thereof is an amount effective to restore the amount an RNA isoform and/or protein isoform of a gene to the amount of the RNA isoform and/or protein isoform detectable in healthy patients or cells from healthy patients.

In some embodiments, an effective amount of a SMSM or a pharmaceutically acceptable salt thereof is an amount effective to decrease the aberrant amount of an RNA transcript of a gene which associated with a disease. In some embodiments, an effective amount of a SMSM or a pharmaceutically acceptable salt thereof is an amount effective to decrease the amount of the aberrant expression of an isoform of a gene. In some embodiments, an effective amount of a SMSM or a pharmaceutically acceptable salt thereof is an amount effective to result in a substantial change in the amount of an RNA transcript (e.g., an mRNA transcript), alternative splice variant, or isoform.

In some embodiments, an effective amount of a SMSM or a pharmaceutically acceptable salt thereof is an amount effective to increase the amount of an RNA transcript (e.g., an mRNA transcript) of a gene that is beneficial for the prevention and/or treatment of a disease. In some embodiments, an effective amount of a SMSM or a pharmaceutically acceptable salt thereof is an amount effective to increase the amount of an alternative splice variant of an RNA transcript of a gene that is beneficial for the prevention and/or treatment of a disease. In some embodiments, an effective amount of a SMSM or a pharmaceutically acceptable salt thereof is an amount effective to increase the amount of an isoform of a gene that is beneficial for the prevention and/or treatment of a disease.

In some embodiments, an effective amount of a SMSM or a pharmaceutically acceptable salt thereof is an amount effective to decrease the amount of an RNA transcript (e.g., an mRNA transcript) which causes or is related to the symptoms of the condition or disease. In particular embodiments, the SMSM decreases the amount of an RNA transcript that causes or relates to the symptoms of the condition or disease by modulating one or more splicing elements of the RNA transcript. In some embodiments, the SMSM promotes skipping of one or more exons. In some embodiments, the SMSM promotes inclusion of one or more exons. In some embodiments, the SMSM promotes inclusion of one or more exons and/or introns that relate to nonsense-mediated mRNA decay (NMD). In some embodiments, the one or more exons harbor a premature termination codon. In particular embodiments, the premature stop codon is an in-frame codon that does not cause frameshift of the downstream exon(s). In some embodiments, inclusion of the one or more exons causes a reading frameshift in a downstream exon, for example, in the immediately downstream exon, introducing a premature termination codon.

A method of treating a disease or a condition in a subject in need thereof can comprise administering to the subject a therapeutically effective amount of a compound described herein or a pharmaceutically acceptable salt thereof. In some embodiments, the present disclosure relates to a method for the treatment, prevention and/or delay of progression of a disease or a condition associated with a gene listed in Table 2.

Non-limiting examples of effective amounts of a SMSM or a pharmaceutically acceptable salt thereof are described herein. For example, the effective amount may be the amount required to prevent and/or treat a disease associated with the aberrant amount of an mRNA transcript of gene in a human subject. In general, the effective amount will be in a range of from about 0.001 mg/kg/day to about 500 mg/kg/day for a patient having a weight in a range of between about 1 kg to about 200 kg. The typical adult subject is expected to have a median weight in a range of between about 70 and about 100 kg.

In one embodiment, a SMSM described herein can be used in the preparation of medicaments for the treatment of diseases or conditions described herein. In addition, a method for treating any of the diseases or conditions described herein in a subject in need of such treatment, can involve administration of pharmaceutical compositions that include at least one SMSM described herein or a pharmaceutically acceptable salt, thereof, in a therapeutically effective amount to a subject.

In certain embodiments, a SMSM described herein can be administered for prophylactic and/or therapeutic treatments. In certain therapeutic applications, the compositions are administered to a patient already suffering from a disease or a condition, in an amount sufficient to cure or at least partially arrest at least one of the symptoms of the disease or the condition. Amounts effective for this use depend on the severity and course of the disease or the condition, previous therapy, the patient's health status, weight, and response to the drugs, and the judgment of the treating physician. Therapeutically effective amounts are optionally determined by methods including, but not limited to, a dose escalation clinical trial. In prophylactic applications, compositions containing a SMSM described herein can be administered to a patient susceptible to or otherwise at risk of a particular disease, disorder, or condition.

### Methods of Administering

The compositions described herein can be administered to the subject in a variety of ways, including parenterally, intravenously, intradermally, intramuscularly, colonically, rectally or intraperitoneally. In some embodiments, the small molecule splicing modulator (SMSM) or a pharmaceutically acceptable salt thereof is administered by intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a small molecule splicing modulator can be in any suitable form for oral administration, such as liquid, tablets, capsules, or the like. The compositions of the present disclosure can be administered to a subject using any suitable methods known in the art. Suitable formulations for use in the present disclosure and methods of delivery are generally well known in the art.

For example, the small molecule splicing modulators described herein can be formulated as pharmaceutical compositions with a pharmaceutically acceptable diluent, carrier, or excipient.

### Dosing and Schedules

The SMSMs utilized in the methods of the disclosure can be, e.g., administered at dosages that may be varied depending upon the requirements of the subject, the severity of the condition being treated and/or imaged, and/or the SMSM being employed. For example, dosages can be empirically determined considering the type and stage of disease diagnosed in a particular subject and/or the type of imaging modality being used in conjunction with the SMSMs. The dose administered to a subject, in the context of the present disclosure should be sufficient to affect a beneficial diagnostic or therapeutic response in the subject. The size of the dose also can be determined by the existence, nature, and extent of any adverse side-effects that accompany the administration of a SMSM in a particular subject.

Within the scope of the present description, the effective amount of a SMSM or a pharmaceutically acceptable salt thereof for use in the manufacture of a medicament, the preparation of a pharmaceutical kit or in a method for preventing and/or treating a disease in a human subject in need thereof, is intended to include an amount in a range of from about 1 μg to about 50 grams.

The compositions of the present disclosure can be administered as frequently as necessary.

### Subjects

The subjects that can be treated with the SMSMs and methods described herein can be any subject that produces mRNA that is subject to alternative splicing, e.g., the subject may be a eukaryotic subject, such as a plant or an animal. In some embodiments, the subject is a mammal, e.g., human. In some embodiments, the subject is a human. In some embodiments, the subject is a non-human animal. In some embodiments, the subject is a fetus, an embryo, or a child. In some embodiments, the subject is a non-human primate such as chimpanzee, and other apes and monkey species; farm animals such as cattle, horses, sheep, goats, swine; domestic animals such as rabbits, dogs, and cats; laboratory animals including rodents, such as rats, mice and guinea pigs, and the like.

In some embodiments, the subject is prenatal (e.g., a fetus), a child (e.g., a neonate, an infant, a toddler, a preadolescent), an adolescent, a pubescent, or an adult (e.g., an early adult, a middle-aged adult, a senior citizen).

### Methods of Making Compounds

Compounds described herein can be synthesized using standard synthetic techniques or using methods known in the art in combination with methods described herein. Unless otherwise indicated, conventional methods of mass spectroscopy, NMR, HPLC, protein chemistry, biochemistry, recombinant DNA techniques and pharmacology can be employed. Compounds can be prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions. The starting materials can be available from commercial sources or can be readily prepared. By way of example only, provided are schemes for preparing the SMSMs described herein.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

SMSMs can be made using known techniques and further chemically modified, in some embodiments, to facilitate intranuclear transfer to, e.g., a splicing complex component, a spliceosome or a pre-mRNA molecule. One of ordinary skill in the art will appreciate the standard medicinal chemistry approaches for chemical modifications for intranuclear transfer (e.g., reducing charge, optimizing size, and/or modifying lipophilicity).

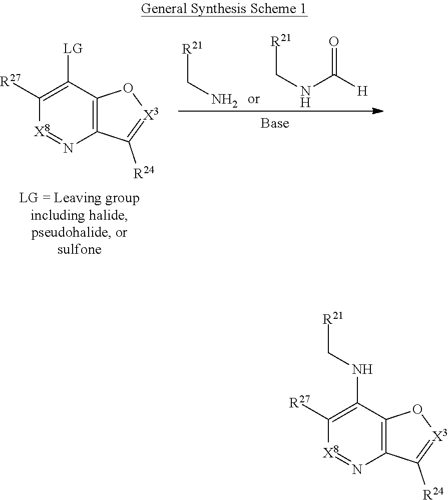

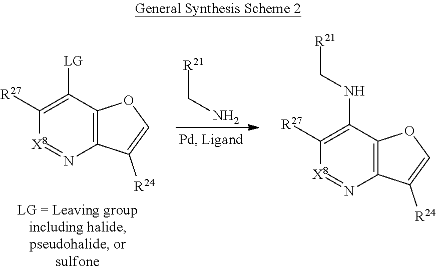

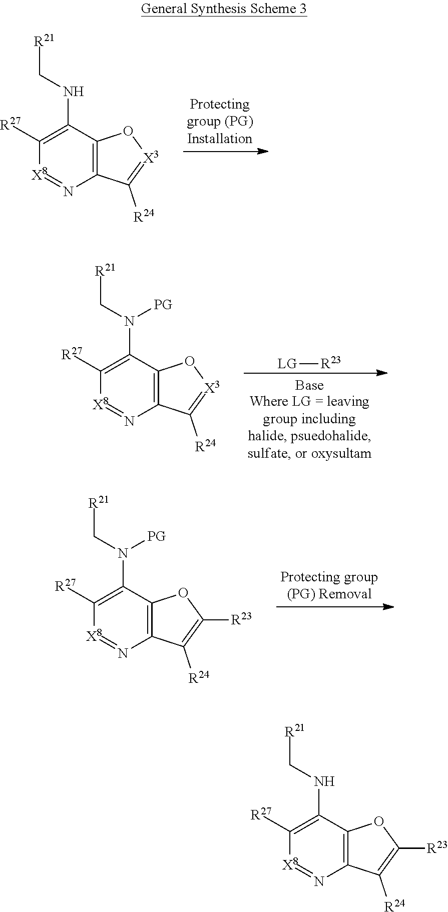

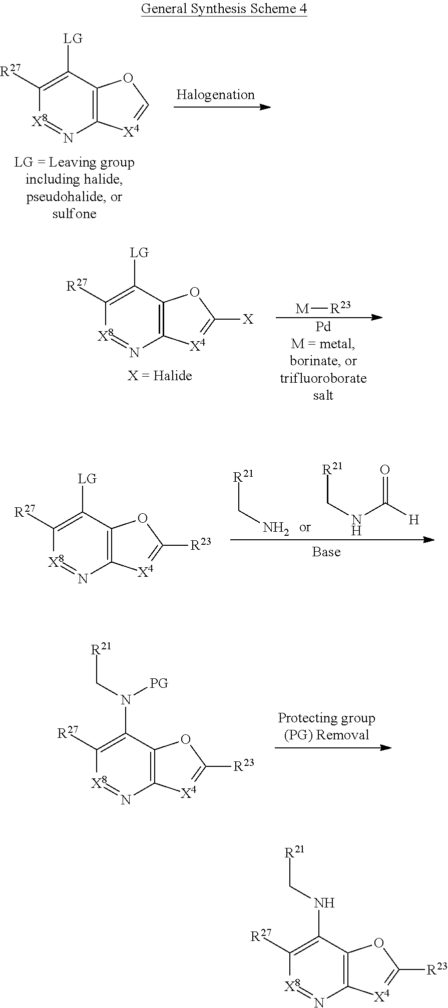

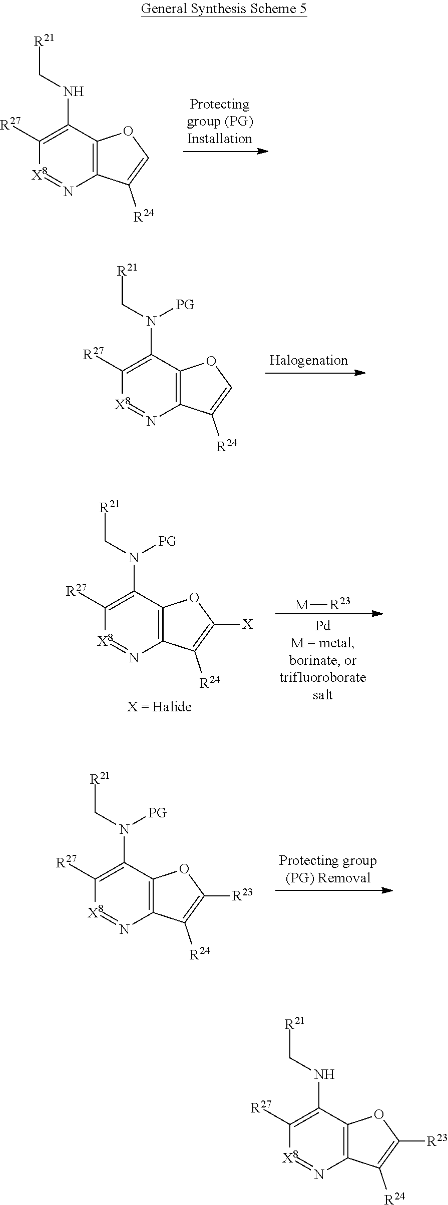

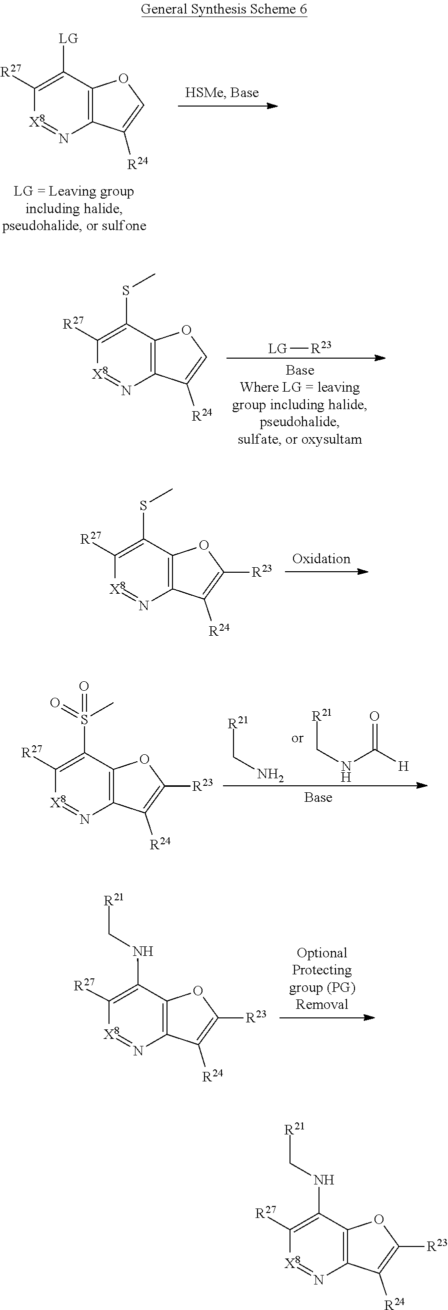

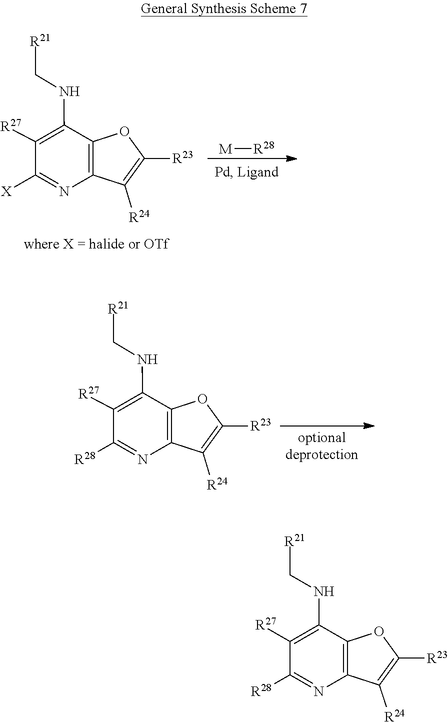

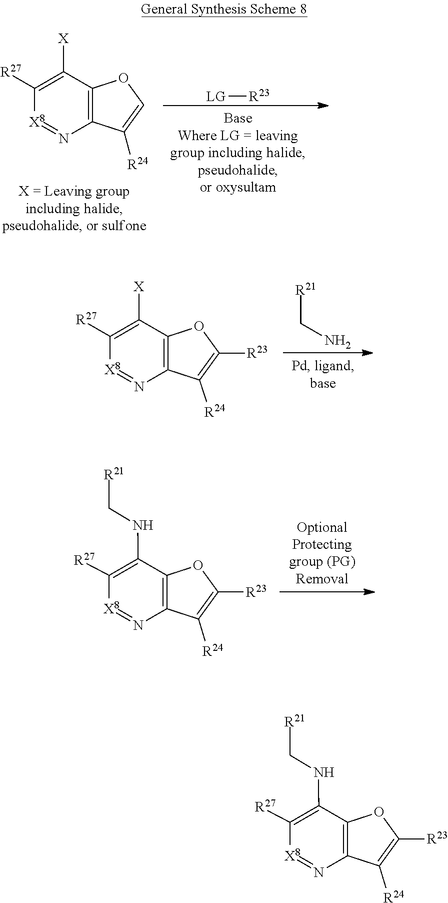

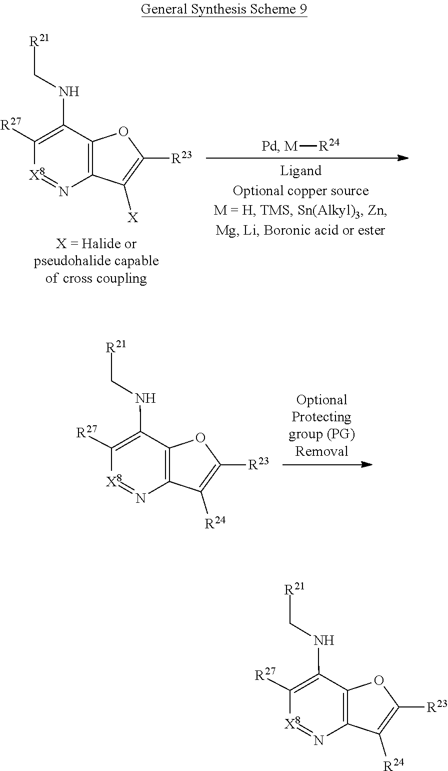

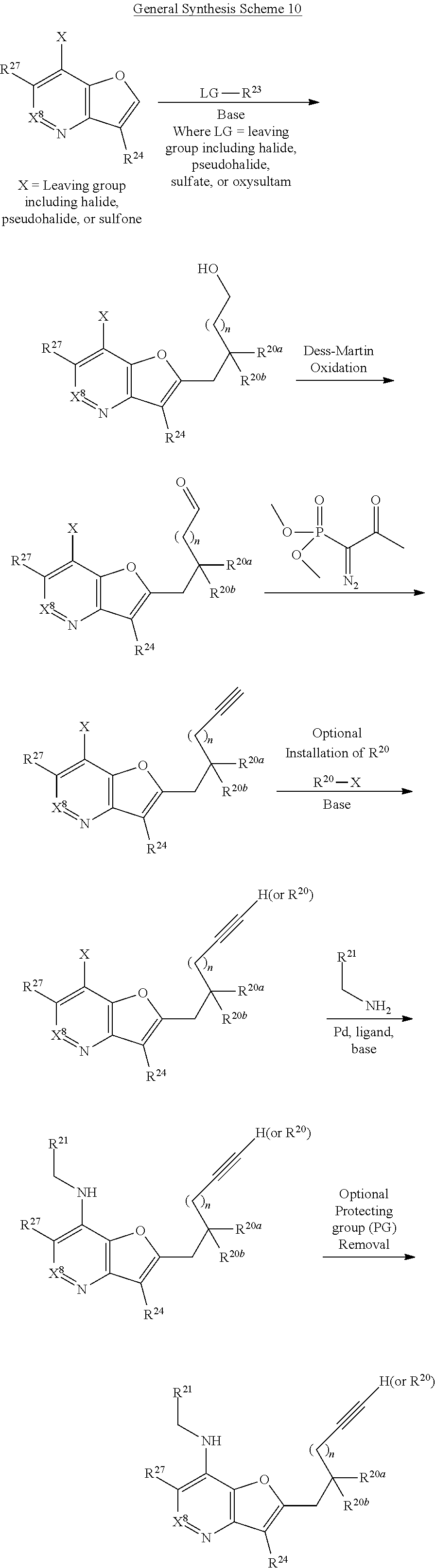

n is e.g., 0, 1, or 2.

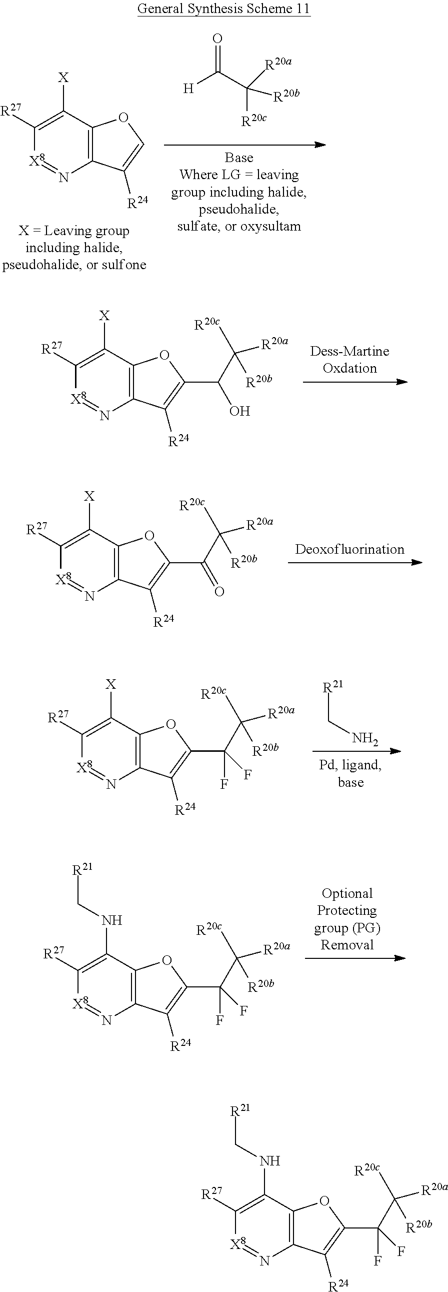

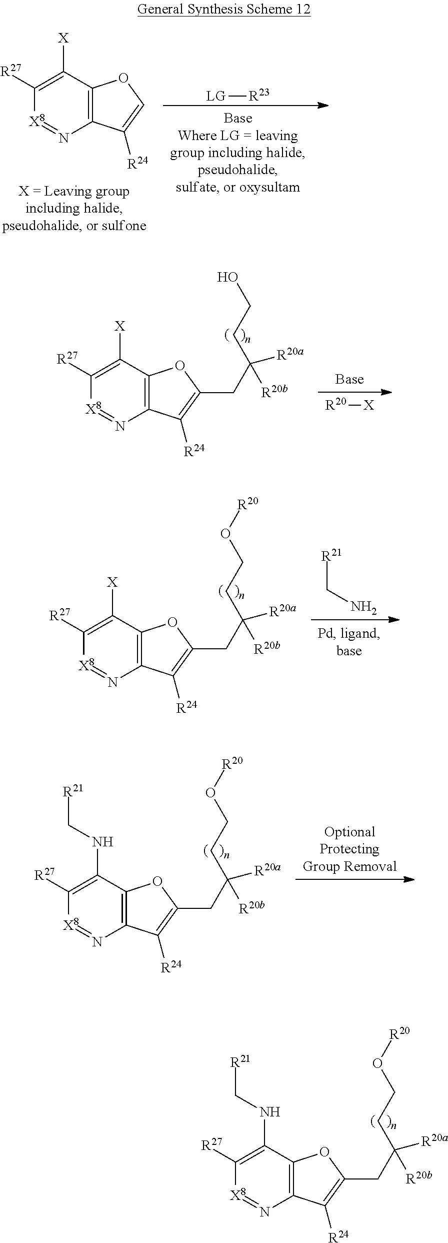

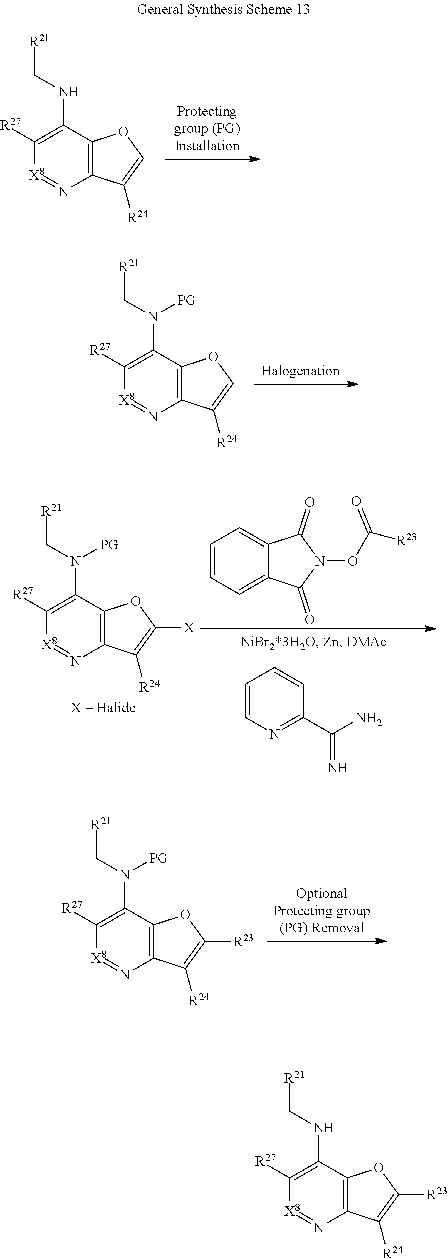

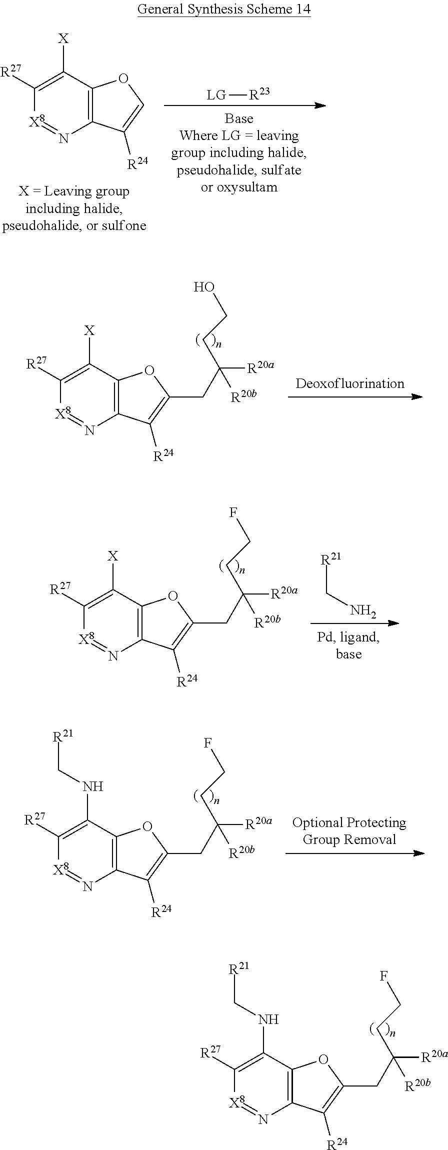

n is e.g., 0, 1 or 2.

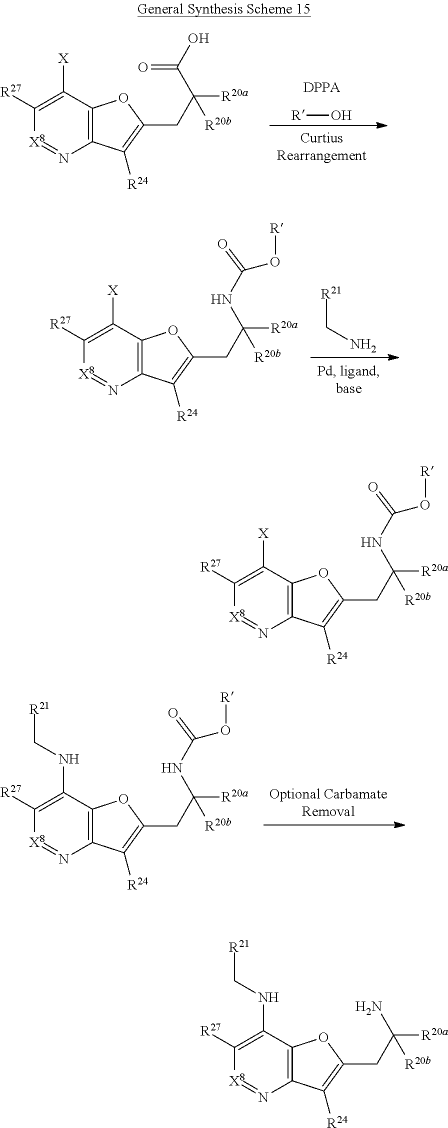

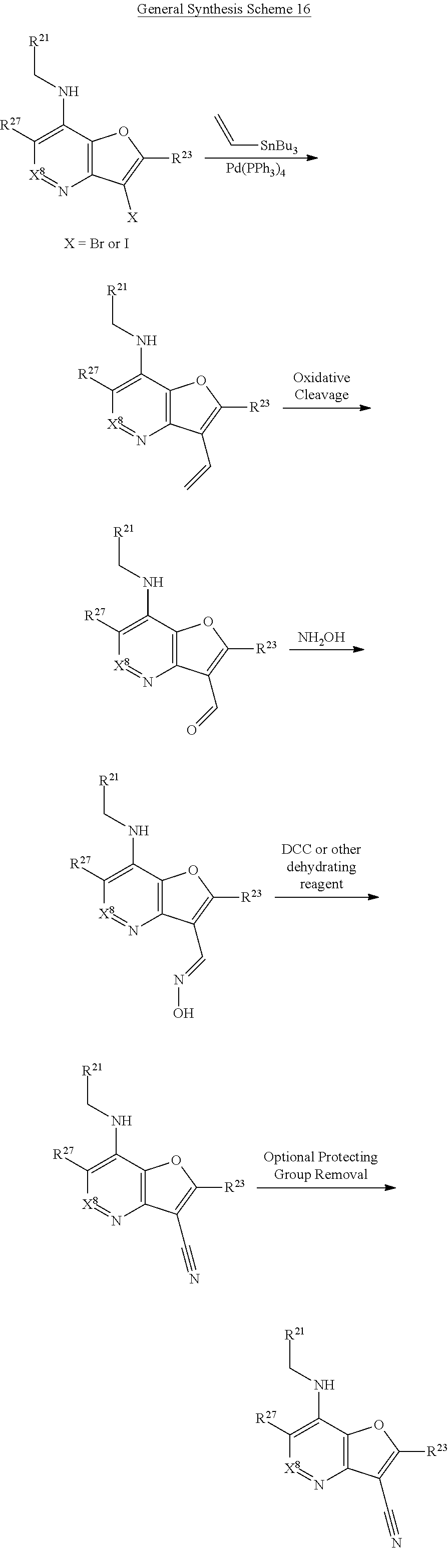

### EXAMPLES

These examples are provided for illustrative purposes only and not to limit the scope of the claims provided herein. The starting materials and reagents used for the synthesis of the compounds described herein may be synthesized or can be obtained from commercial sources, such as, but not limited to, Sigma-Aldrich, Acros Organics, Fluka, and Fisher Scientific.

### Example 1. References and Syntheses of Common Building Blocks and Starting Materials

### General Procedure for the Synthesis of Cyclic Sulfamidates AA:

Cyclic Sulfamidates can be synthesized by the following general method starting from the appropriate amino alcohol.

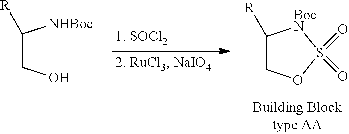

Additional synthetic procedures and/or literature references for known cyclic sulfamidates are provided below.

Reference for tert-butyl (S)-4-methyl-1,2,3-oxathiazolidine-3-carboxylate 2,2-dioxide (AA-1) Bower, J. F., Szetzo, P.; Gallagher, T. Org. Lett. 2007, 9, 3283-3286.

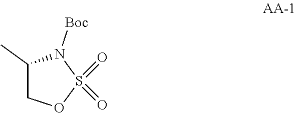

### Synthesis of Tert-butyl (R)-4-((S)-1-fluoroethyl)-1,2,3-oxathiazolidine-3-carboxylate 2,2-dioxide (AA-2)

### Step 1: Synthesis of 3-(tert-butyl) 4-methyl (4S,5R)-5-methyl-1,2,3-oxathiazolidine-3,4-dicarboxylate2,2-dioxide

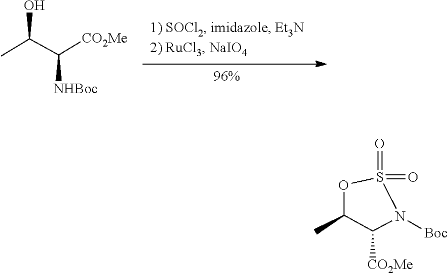 [SMILES: COC(=O)[C@@H](NC(=O)OC(C)(C)C)[C@@H](C)O ; COC(=O)[C@@H]1[C@@H](C)OS(=O)(=O)N1C(=O)OC(C)(C)C]

To a solution of imidazole (3.62 g, 4 Eq, 53.2 mmol), triethylamine (3.36 g, 4.63 mL, 2.5 Eq, 33.2 mmol) in DCM (60 mL) at −60° C. was added dropwise thionyl chloride (1.74 g, 1.07 mL, 1.10 Eq, 14.6 mmol) followed by methyl (tert-butoxycarbonyl)-L-threoninate (3.10 g, 1 Eq, 13.3 mmol) in DCM (30.00 mL) keeping the reaction mixture below −55° C. The turbid mixture was allowed to warm to rt slowly and stirred 30 minutes. The reaction was quenched with 0.5N HCl (150 mL) and the water layer was extracted with DCM (2×75 mL). The combined organic layers were washed with half brine and half water, dried over sodium sulfate, filtered, and concentrated in vacuo.

The crude mixture was redissolved in MeCN (50 mL), cooled to 0° C., and treated with sodium periodate (3.27 g, 1.15 Eq, 15.3 mmol) followed by ruthenium trichloride (276 mg, 88.6 μL, 0.1 Eq, 1.33 mmol) and water (50 mL). The reaction was stirred for 1 h at 0° C. and diluted with water and TBME and filtered through a pad of celite. The water layer was extracted with TBME two times. The combined organic layers were washed with brine, dried over sodium sulfate, filtered and concentrated in vacuo to afford 3-(tert-butyl) 4-methyl (4S,5R)-5-methyl-1,2,3-oxathiazolidine-3,4-dicarboxylate 2,2-dioxide (3.770 g, 12.77 mmol, 96.1%).

1H NMR (299 MHz, CDCl₃) δ 5.06-4.72 (m, 1H), 4.51 (dd, J=5.8, 1.4 Hz, 1H), 3.87 (s, 3H), 1.73 (d, J=6.4 Hz, 3H), 1.57 (s, 9H).

### Step 2: Synthesis of methyl (2R,3S)-2-((tert-butoxycarbonyl)amino)-3-fluorobutanoate

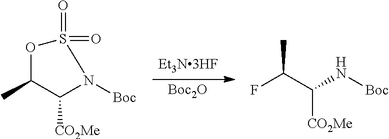 [SMILES: "Boc2O" ; COC(=O)[C@@H]1[C@@H](C)OS(=O)(=O)N1C(=O)OC(C)(C)C ; COC(=O)[C@@H](NC(=O)OC(C)(C)C)[C@H](C)F]

To a solution of 3-(tert-butyl) 4-methyl (4S,5R)-5-methyl-1,2,3-oxathiazolidine-3,4-dicarboxylate 2,2-dioxide (5.770 g, 1 Eq, 19.54 mmol) in THE (100.00 mL) was added triethylamine trihydrofluoride (20.47 g, 20.7 mL, 6.50 Eq, 127.0 mmol) and the reaction mixture was refluxed for 16 h. The reaction was neutralized with a saturated solution of sodium bicarbonate until the pH tested basic. Next, boc anhydride (4.264 g, 1 eq, 19.54 mmol) was added in one portion. The reaction mixture was stirred for 30 minutes at room temperature and extracted with EtOAc twice. The combined organic layers were washed with water and brine, dried over sodium sulfate, filtered and concentrated in vacuo. The crude material was purified by column chromatography (Heptanes/EtOAc 90/10) to afford methyl (2R,3S)-2-((tert-butoxycarbonyl)amino)-3-fluorobutanoate (3.150 g, 13.39 mmol, 68.53%).

¹H NMR (299 MHz, CDCl₃) δ 5.38 (s, 1H), 4.88 (ddd, J=47.2, 6.4, 3.7 Hz, 1H), 4.47 (dd, J=22.2, 9.0 Hz, 1H), 3.82 (s, 3H), 1.51-1.36 (m, 12H).

### Step 3: Synthesis of Tert-butyl ((2R,3S)-3-fluoro-1-hydroxybutan-2-yl)carbamate

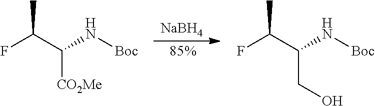 [SMILES: COC(=O)[C@@H](NC(=O)OC(C)(C)C)[C@H](C)F ; "NaBH4" ; "85%" ; C[C@H](F)[C@@H](CO)NC(=O)OC(C)(C)C]

To a solution of methyl (2R,3S)-2-((tert-butoxycarbonyl)amino)-3-fluorobutanoate (3.08 g, 1 Eq, 13.1 mmol) in EtOH (65.00 mL) at 0° C. was added NaBH₄ (1.24 g, 2.5 Eq, 32.7 mmol). The mixture was stirred at 0° C. for 8 h. The mixture was poured into 100 mL of water. The water layer was extracted with DCM (3×100 mL). The combined organic layers were washed with brine, dried over sodium sulfate, filtered and concentrated in vacuo. The crude material was purified by column chromatography (500 mL silica, Heptanes/EA, 65/35 to afford tert-butyl ((2R,3S)-3-fluoro-1-hydroxybutan-2-yl)carbamate (2.30 g, 11.1 mmol, 84.8%).

¹H NMR (299 MHz, CDCl3) δ 5.12 (s, 1H), 4.80 (dt, J=48.0, 6.1 Hz, 1H), 4.04-3.88 (m, 1H), 3.85-3.59 (m, 2H), 1.97 (s, 1H), 1.56-1.33 (m, 12H).

### Step 4: Tert-butyl (R)-4-((S)-1-fluoroethyl)-1,2,3-oxathiazolidine-3-carboxylate 2,2-dioxide

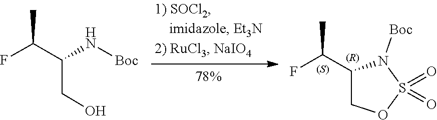

To a solution of imidazole (3.02 g, 4 Eq, 44.4 mmol), triethylamine (2.81 g, 3.87 mL, 2.5 Eq, 27.7 mmol) in DCM (60.00 mL) at −60° C. was added dropwise thionyl chloride (1.45 g, 891 μL, 1.1 Eq, 12.2 mmol) followed by tert-butyl ((2R,3S)-3-fluoro-1-hydroxybutan-2-yl)carbamate (2.30 g, 1 Eq, 11.1 mmol) in DCM (30.00 mL) while keeping the temperature of the reaction mixture below −55° C. The turbid mixture was allowed to warm to rt slowly and stirred for 30 minutes. The reaction was quenched with 0.5N HCl (150 mL) and the phases were separated. The water layer was extracted with DCM (2×75 mL). The combined organic layers washed brine (100 mL), dried over sodium sulfate, filtered, and concentrated in vacuo. To a solution of the crude material in MeCN (40.00 mL) at 0° C. was added sodium periodate (2.73 g, 1.15 Eq, 12.8 mmol) followed by ruthenium trichloride (230 mg, 74.0 μL, 0.1 Eq, 1.11 mmol) and water (40.00 mL). The reaction was stirred 1 h at 0° C. and diluted with water (50 mL) and TBME (150 mL) and filtered through a pad of celite. The celite cake was washed with 50 mL of TBME. The water layer was extracted with TBME twice (2×75 mL). The combined organic layers were washed with brine (100 mL), dried over sodium sulfate, filtered and concentrated in vacuo. The crude material was purified by column chromatography (500 mL silica, Hept/EtOAc, 80/20 to 75/25) to afford tert-butyl (R)-4-((S)-1-fluoroethyl)-1,2,3-oxathiazolidine-3-carboxylate 2,2-dioxide (2.32 g, 8.62 mmol, 77.6%) as a white solid. ¹H NMR (299 MHz, CDCl₃) δ 4.95 (d of quintets, J=47.5, 6.3 Hz, 1H), 4.79-4.56 (m, 2H), 4.33 (dtd, J=13.7, 5.5, 2.5 Hz, 1H), 1.58 (s, 9H), 1.43 (d, J=24.1, 6.4, 1.1 Hz, 3H).

Reference for tert-butyl (S)-4-(cyclopropylmethyl)-1,2,3-oxathiazolidine-3-carboxylate 2,2-dioxide (AA-3) (WO2022/042657).

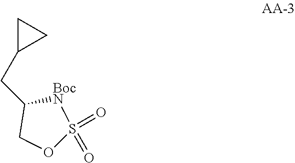 [SMILES: CC(C)(C)OC(=O)N1[C@@H](CC2CC2)COS1(=O)=O]

Reference for tert-butyl (S)-4-(((tert-butyldimethylsilyl)oxy)methyl)-1,2,3-oxathiazolidine-3-carboxylate 2,2-dioxide (AA-4) Hebeisen, P.; Weiss, U.; Alker, A.; Staempfli, A. Tetrahedron Lett., 2011, 52, 5229-5233.

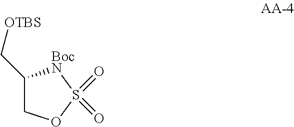 [SMILES: CC(C)(C)OC(=O)N1[C@@H](CO[Si](C)(C)C(C)(C)C)COS1(=O)=O]

### Synthesis of Tert-butyl (R)-4-(difluoromethyl)-1,2,3-oxathiazolidine-3-carboxylate 2,2-dioxide (AA-5)

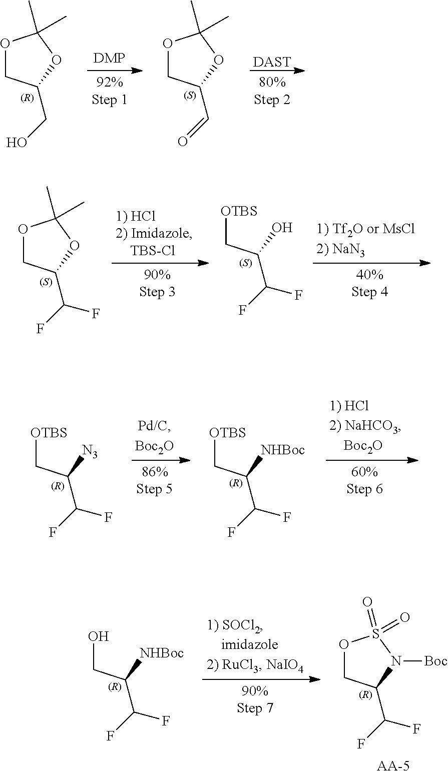

### Step 1. Synthesis of (S)-2,2-dimethyl-1,3-dioxolane-4-carbaldehyde

(R)-(2,2-dimethyl-1,3-dioxolan-4-yl)methanol (23.5 mL, 1 Eq, 189 mmol) was dissolved in DCM (800 mL) and sodium bicarbonate (79.5 g, 5 Eq, 946 mmol) was added. To the stirring suspension was then added, Dess-Martin periodinane (DMP) (120 g, 1.5 Eq, 284 mmol). After 4 hours, the mixture was quenched with 500 ml of water and sodium thiosulfate (150 g, 5 Eq, 946 mmol). The mixture was stirred for approximately 15 minutes until a non-cloudy mixture was obtained. The layers were then separated, and the aqueous layer was extracted three times with 300 mL of DCM. The combined organic layers were then dried over Na₂SO₄ and concentrated to afford 24.0 g of a crude pale-yellow oil. The crude oil was filtered and residue washed with diethyl ether. The filtrate was concentrated to afford (S)-2,2-dimethyl-1,3-dioxolane-4-carbaldehyde (22.5 g, 173 mmol, 91.4%) as pale-yellow oil.

### Step 2. Synthesis of (S)-4-(difluoromethyl)-2,2-dimethyl-1,3-dioxolane

(S)-2,2-dimethyl-1,3-dioxolane-4-carbaldehyde (10.0 g, 1 Eq, 76.8 mmol) was dissolved in DCM (125 mL) and DAST (14.2 mL, 1.4 Eq, 108 mmol) was added dropwise at 0° C. The mixture was allowed to warm up to rt and stirred for 3 hours. The mixture was quenched with sat. aq. sodium bicarbonate solution with cooling in an ice-water bath. The layers were then separated, and the aqueous layer was extracted twice with 100 mL of DCM. The combined organic layers were washed with brine, dried over Na₂SO₄, and concentrated to afford (S)-4-(difluoromethyl)-2,2-dimethyl-1,3-dioxolane (9.34 g, 61.4 mmol, 79.9%) as yellow oil.

### Step 3. Synthesis of (S)-3-((tert-butyldimethylsilyl)oxy)-1,1-difluoropropan-2-ol

(S)-4-(difluoromethyl)-2,2-dimethyl-1,3-dioxolane (2.6 g, 1 Eq, 17 mmol) was dissolved in MeOH (30 mL) and HCl in diethyl ether (13 mL, 2 molar, 1.5 Eq, 26 mmol) was added at 0° C. The resulting mixture was stirred overnight at rt. After stirring overnight, TLC (40% EtOAc in heptane) indicated the formation of one new product with an R_{f} of 0.2 and the complete disappearance of the starting material at R_{f}=0.95. The mixture was concentrated and this resulted in (S)-3,3-difluoropropane-1,2-diol (1.62 g, 14.5 mmol, 85%) as pale green oil.

(S)-3,3-difluoropropane-1,2-diol (1.46 g, 1 Eq, 13.0 mmol) was dissolved in DMF (45 mL) and imidazole (1.95 g, 2.2 Eq, 28.7 mmol) was added. The mixture was cooled down to 0° C. and of tert-butylchlorodimethylsilane (2.00 g, 1.02 Eq, 13.3 mmol) was then added. After two hours the mixture was diluted with 60 mL of TBME and 60 mL of water. The layers were separated, and the water layer was extracted two more times with 40 mL of TBME. The combined organic layers were then washed with a small amount (5 mL) of water (2×). The combined organic layers were then dried over Na₂SO₄ and concentrated to afford (S)-3-((tert-butyldimethylsilyl)oxy)-1,1-difluoropropan-2-ol (2.68 g, 11.8 mmol, 90.9%) as pale-yellow oil.

### Step 4. Synthesis of (R)-(2-azido-3,3-difluoropropoxy)(tert-butyl)dimethylsilane

(S)-3-((tert-butyldimethylsilyl)oxy)-1,1-difluoropropan-2-ol (1.65 g, 1 Eq, 7.29 mmol) was dissolved in DCM (50 mL) and cooled down to −20° C. using an acetonitrile/dry ice bath. Next, 2,6-lutidine (1.69 mL, 2 Eq, 14.6 mmol) was added followed by the drop-wise addition of triflic anhydride (1.42 mL, 1.15 Eq, 8.38 mmol) (caution: exothermic). The mixture was stirred at −20° C. during the addition (20 minutes) followed by 60 minutes at −10° C. and two hours at 0° C. The mixture was diluted with diethyl ether (100 ml) and washed with 15 mL each of water, 1M HCl, sat. aq. bicarbonate and brine. The organic phase was dried over Na₂SO₄ and concentrated to afford (S)-3-((tert-butyldimethylsilyl)oxy)-1,1-difluoropropan-2-yl trifluoromethanesulfonate (2.9 g, 8.1 mmol, 110%) as orange oil.

### Step 5. Synthesis of tert-butyl (R)-(3-((tert-butyldimethylsilyl)oxy)-1,1-difluoropropan-2-yl)carbamate

(R)-(2-azido-3,3-difluoropropoxy)(tert-butyl)dimethylsilane (280 mg, 1 Eq, 1.11 mmol) was left to stir at 50° C. in EtOAc (10 mL) with Pd(OH)₂ (46.9 mg, 0.3 Eq, 334 μmol) and Boc₂O (267 mg, 282 μL, 1.1 Eq, 1.23 mmol) in a hydrogen atmosphere overnight. The mixture was then cooled to rt, diluted with 50 mL of EtOAc and filtered over Celite®. The filtrate was dried over Na₂SO₄ and concentrated to afford 400 mg of a colorless oil. The crude was purified on 12 g of silica using 0-20% EtOAc in heptane to afford tert-butyl (R)-(3-((tert-butyldimethylsilyl)oxy)-1,1-difluoropropan-2-yl)carbamate (310 mg, 952 μmol, 85.5%) as a colorless oil.

### Step 6. Synthesis of Tert-butyl (R)-(1,1-difluoro-3-hydroxypropan-2-yl)carbamate

Tert-butyl (R)-(3-((tert-butyldimethylsilyl)oxy)-1,1-difluoropropan-2-yl)carbamate (300 mg, 1 Eq, 922 mol) was dissolved in THE (5 mL) and water (5 mL). Then, HCl (1.38 mL, 4 molar, 6 Eq, 5.53 mmol) was added and mixture was stirred overnight. Next morning, mixture was neutralized with sodium hydrogen carbonate (619 mg, 8 Eq, 7.37 mmol) and subsequently di-tert-butyl dicarbonate (201 mg, 1 Eq, 922 mol) was added. After 1.5 hours, the mixture was extracted with 2×10 mL of EtOAc, washed with 5 mL of 0.5N HCl, 5 mL of sat. aq. sodium bicarbonate solution, dried over Na₂SO₄, filtered, and concentrated to afford 300 mg of a crude colorless oil. Purification by silica gel chromatography afford tert-butyl (R)-(1,1-difluoro-3-hydroxypropan-2-yl)carbamate (100 mg, 473 mol, 51.4%) as colorless oil.

### Step 7. Synthesis of Tert-butyl (R)-4-(difluoromethyl)-1,2,3-oxathiazolidine-3-carboxylate 2,2-dioxide (AA-5)

To a solution of imidazole (2.32 g, 4 Eq, 34.1 mmol) and triethylamine (2.97 mL, 2.5 Eq, 21.3 mmol) in DCM (60 mL) at −60° C. was added thionyl chloride (1.22 g, 746 μL, 1.2 Eq, 10.2 mmol) dropwise. Subsequently, tert-butyl (R)-(1,1-difluoro-3-hydroxypropan-2-yl)carbamate (1.80 g, 1 Eq, 8.52 mmol) in DCM (15 mL) was added in a slow stream keeping the reaction around −60° C. The reaction was allowed to warm to RT and stirred over the course of 1 hour and monitored by TLC. The TLC (20% EtOAc in heptane) indicated the complete conversion of the starting material (R_{f}=0.5) to one major product (R_{f}=0.45). The mixture was quenched with 10 ml of 0.5M HCl, separated and the aq. layer was extracted twice more with 10 mL of DCM. The combined organic layers were dried over Na₂SO₄ and concentrated to afford the desired cyclized product tert-butyl (4R)-4-(difluoromethyl)-1,2,3-oxathiazolidine-3-carboxylate 2-oxide (2.00 g, 7.77 mmol, 91.2%) as colorless oil.

The oil thus obtained was then dissolved in MeCN (15 mL) and cooled to 0° C. A portion of sodium periodate (2.10 g, 1.15 Eq, 9.80 mmol) was added, followed by ruthenium(III) chloride (177 mg, 0.1 Eq, 852 mol) and water (15 mL). The mixture was stirred at 0° C. for 75 minutes. After this time, the black/intense red mixture was diluted with 15 mL of TBME and 15 mL of water and filtered on Celite. The filter cake was washed with 3×20 ml of TBME and the organic and aqueous layers were separated. The organic layer was washed with brine, dried over Na₂SO₄, filtered and concentrated to afford tert-butyl (R)-4-(difluoromethyl)-1,2,3-oxathiazolidine-3-carboxylate 2,2-dioxide (2.00 g, 7.32 mmol, 85.9%) as white crystalline material. ¹H NMR (299 MHz, CDCl₃) δ 6.14 (ddt, J=56.3, 54.1, 2.1 Hz, 1H), 4.79 (m, 1H), 4.69 (m, 1H), 4.61-4.47 (m, 1H), 1.57 (s, 9H).

Reference for tert-butyl (S)-4-(2-((tert-butyldimethylsilyl)oxy)ethyl)-1,2,3-oxathiazolidine-3-carboxylate 2,2-dioxide (AA-6): Zhang, N.; Arnold, M. A.; Dakka, A.; Karp, G. M.; Luong, T. T.; Narasimhan, J.; Naryshkin, N. A.; Wang, J.; Zhang, X. (WO2020167628).

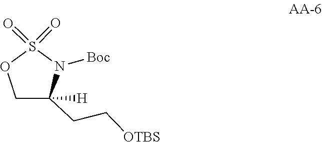 [SMILES: CC(C)(C)OC(=O)N1[C@@H](CCO[Si](C)(C)C(C)(C)C)COS1(=O)=O]

Reference for tert-butyl (S)-4-(methoxymethyl)-1,2,3-oxathiazolidine-3-carboxylate 2,2-dioxide (AA-7) (WO2017/080979A1).

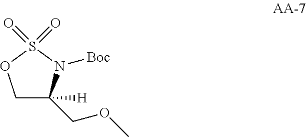 [SMILES: COC[C@H]1COS(=O)(=O)N1C(=O)OC(C)(C)C]

Reference for tert-butyl (R)-4-(fluoromethyl)-1,2,3-oxathiazolidine-3-carboxylate 2,2-dioxide (AA-8) (WO2020/167628A1).

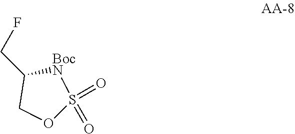 [SMILES: CC(C)(C)OC(=O)N1[C@@H](CF)COS1(=O)=O]

Tert-butyl (S)-4-(2,2,2-trifluoroethyl)-1,2,3-oxathiazolidine-3-carboxylate 2,2-dioxide (AA-9) was synthesized from tert-butyl (S)-(4,4,4-trifluoro-1-hydroxybutan-2-yl)carbamate (WO2012/116279) according to the general method

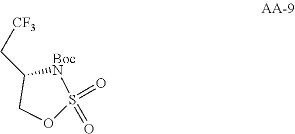 [SMILES: CC(C)(C)OC(=O)N1[C@@H](CC(F)(F)F)COS1(=O)=O]

Reference for tert-butyl 1,2,3-oxathiazinane-3-carboxylate 2,2-dioxide (AA-10) Moss, T. A.; Alonso, B.; Denwick, D. R.; Dixon, D. J. Angew. Chem. Int. Ed. 2010, 49, 568-571.

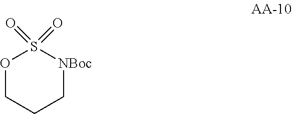 [SMILES: CC(C)(C)OC(=O)N1CCCOS1(=O)=O]

### Synthesis of tert-butyl 4-(oxetan-3-yl)-2,2-dioxo-1,2lambda6,3-oxathiazolidine-3-carboxylate (AA-11)

### Step 1.

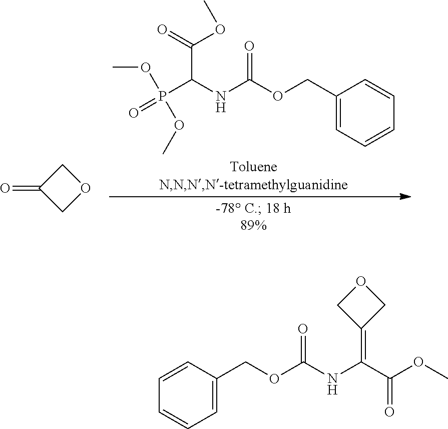 [SMILES: COC(=O)C(NC(=O)OCc1ccccc1)P(=O)(OC)OC ; O=C1COC1 ; COC(=O)C(NC(=O)OCc1ccccc1)=C1COC1]

To a stirred solution of methyl 2-{[(benzyloxy)carbonyl]amino}-2-(dimethoxyphosphoryl)acetate (10 g, 30.188 mmol, 1 equiv) in toluene (100 mL) under an nitrogen atmosphere was added 1,1,3,3-tetramethylguanidine (3.48 g, 30.188 mmol, 1 equiv) at −78° C. and stirred for 30 min. Then a solution of 3-oxetanone (2.18 g, 30.188 mmol, 1 equiv) in toluene (10 mL) was added to the reaction mixture at −78° C. The reaction mixture was warmed to room temperature and stirred for 18 h. After consumption of the starting material (monitored by TLC), the reaction mixture was diluted with water (250 mL) and extracted with EtOAc (2×250 mL) and 20% MeOH:CH₂Cl₂ (2×250 mL). The combined organic extracts were dried over sodium sulfate, filtered and concentrated in vacuo. The crude material was purified by column chromatography using 5-10% MeOH:CH₂Cl₂ to afford methyl 2-{[(benzyloxy)carbonyl]amino}-2-(oxetan-3-ylidene)acetate (7.45 g, 89.00%) as a white solid.

### Step 2.

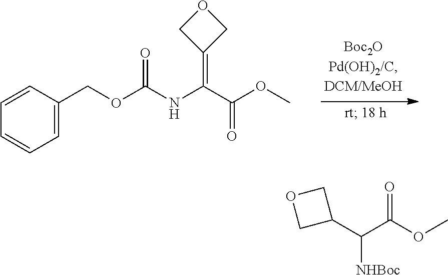 [SMILES: COC(=O)C(NC(=O)OCc1ccccc1)=C1COC1 ; COC(=O)C(NC(=O)OC(C)(C)C)C1COC1]

A solution of methyl 2-{[(benzyloxy)carbonyl]amino}-2-(oxetan-3-ylidene)acetate (4 g, 14.426 mmol, 1 equiv) in DCM/MeOH (1/1; 50 mL) was treated with Pd(OH)₂/C (800 mg, 5.697 mmol, 0.39 equiv) and Boc₂O (3.15 g, 14.426 mmol, 1 equiv). The resulting mixture was stirred for 18 h at room temperature. The reaction was monitored by LCMS. The residue was purified by reverse flash chromatography with the following conditions: column, C18 silica gel; mobile phase, MeCN in Water (10 mmol/L NH₄HCO₃), 10% to 50% gradient in 10 min; detector, UV 254 nm. This resulted in methyl 2-[(tert-butoxycarbonyl)amino]-2-(oxetan-3-yl)acetate (1.3 g, 36.74%) as a yellow oil.

### Step 3.

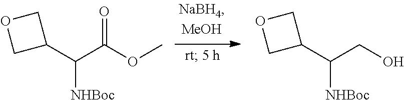 [SMILES: COC(=O)C(NC(=O)OC(C)(C)C)C1COC1 ; CC(C)(C)OC(=O)NC(CO)C1COC1]

A solution of methyl 2-[(tert-butoxycarbonyl)amino]-2-(oxetan-3-yl)acetate (1.3 g, 5.300 mmol, 1 equiv) in MeOH (13.00 mL) was treated with NaBH₄ (0.60 g, 15.900 mmol, 3 equiv) at 0° C. The resulting mixture was stirred for 5 h at room temperature. The reaction was monitored by LCMS. The residue was purified by silica gel column chromatography, eluted with PE/EA (1/1) to afford tert-butyl N-[2-hydroxy-1-(oxetan-3-yl)ethyl]carbamate (700 mg, 60.79%) as a yellow oil.

### Step 4.

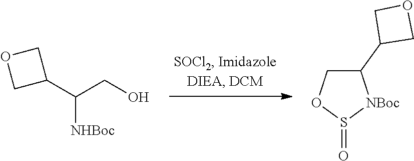 [SMILES: CC(C)(C)OC(=O)NC(CO)C1COC1 ; CC(C)(C)OC(=O)N1C(C2COC2)COS1=O]

To a solution of Imidazole (1.00 g, 14.728 mmol, 4 equiv) in DCM (24 mL) were added SOCl₂ (0.40 mL, 5.523 mmol, 1.5 equiv) followed by DIEA (1.28 mL, 7.364 mmol, 2 equiv) dropwise at 0° C. To the above mixture was added tert-butyl N-[2-hydroxy-1-(oxetan-3-yl)ethyl]carbamate (800 mg, 3.682 mmol, 1 equiv) dissolved in DCM (4 mL) dropwise at 0° C. The resulting mixture was stirred for additional 1 h at room temperature. The resulting mixture was washed with 3×20 mL of conc. HCl (1M). The resulting mixture was concentrated under vacuum. The residue was purified by silica gel column chromatography, eluted with PE/EA (1:1) to afford tert-butyl 4-(oxetan-3-yl)-2-oxo-1,2lambda4,3-oxathiazolidine-3-carboxylate (780 mg, 80.45%) as a yellow oil.

### Step 5.

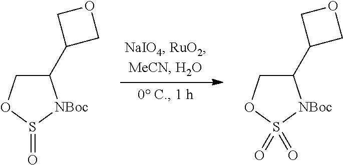 [SMILES: CC(C)(C)OC(=O)N1C(C2COC2)COS1=O ; CC(C)(C)OC(=O)N1C(C2COC2)COS1(=O)=O]

To a solution of tert-butyl 4-(oxetan-3-yl)-2-oxo-1,2lambda4,3-oxathiazolidine-3-carboxylate (780 mg, 2.962 mmol, 1 equiv) in MeCN (10 mL) and H₂O (6 mL) was added NaIO4 (760.32 mg, 3.554 mmol, 1.20 equiv) and ruthenium(iv) oxide hydrate (8.95 mg, 0.059 mmol, 0.02 equiv) at 0° C. The resulting mixture was stirred for 1 h at 0° C. The resulting mixture was filtered through a Celite pad. The filtrate was extracted with EtOAc (3×20 mL). The combined organic layers were washed with brine (20 mL), dried over anhydrous Na2SO4. After filtration, the filtrate was concentrated under reduced pressure. The residue was purified by silica gel column chromatography, eluted with PE/EA (1:1) to afford tert-butyl 4-(oxetan-3-yl)-2,2-dioxo-1,2lambda6,3-oxathiazolidine-3-carboxylate (470 mg, 56.80%) as a white solid.

Synthesis of tert-butyl (S)-4-(3,3-difluorocyclobutyl)-1,2,3-oxathiazolidine-3-carboxylate 2,2-dioxide (AA-12): Compound AA-12 was made from tert-butyl N-[(1S)-1-(3,3-difluorocyclobutyl)-2-hydroxyethyl]carbamate following the general procedure.

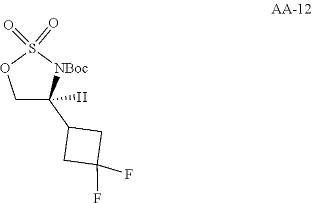 [SMILES: CC(C)(C)OC(=O)N1[C@@H](C2CC(F)(F)C2)COS1(=O)=O]

### Synthesis of tert-butyl N-[(1S)-1-(3,3-difluorocyclobutyl)-2-hydroxyethyl]carbamate

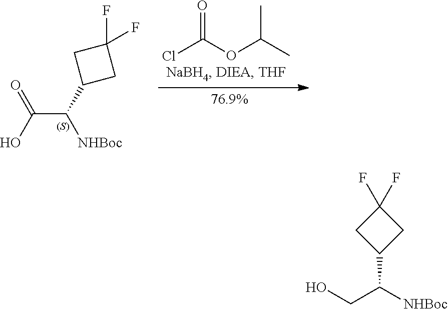

A solution of (S)-[(tert-butoxycarbonyl)amino](3,3-difluorocyclobutyl)acetic acid (4.8 g, 18.096 mmol, 1 equiv) in THF (50 mL) was treated with DIEA (4.68 g, 36.192 mmol, 2 equiv) and isopropyl chloroformate (2.88 g, 23.525 mmol, 1.3 equiv) at 0° C. for 30 min under nitrogen atmosphere followed by the addition of NaBH₄ (2.74 g, 72.384 mmol, 4 equiv) in portions at 0° C. The resulting mixture was stirred for 2 h at room temperature. The reaction was quenched with water at 0° C. The resulting mixture was extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with PE/EA (5:1) to afford tert-butyl N-[(1S)-1-(3,3-difluorocyclobutyl)-2-hydroxyethyl]carbamate (3.5 g, 76.97%) as a colorless oil.

### Synthesis of tert-butyl (S)-4-(2,2-difluoroethyl)-1,2,3-oxathiazolidine-3-carboxylate AA-13)

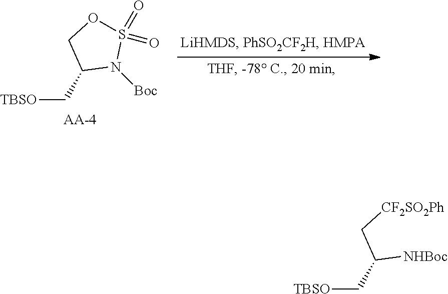

### Step 1: tert-butyl (S)-(1-((tert-butyldimethylsilyl)oxy)-4,4-difluoro-4-(phenylsulfonyl)butan-2-yl)carbamate

LiHMDS (1M solution in THF, 16.33 mL, 16.3 mmol, 1.2 equiv) was added slowly to a solution of tert-butyl (R)-4-(((tert-butyldimethylsilyl)oxy)methyl)-1,2,3-oxathiazolidine-3-carboxylate 2,2-dioxide (5 g, 13.6 mmol, 1 equiv) and (difluoromethane)sulfonylbenzene (2.13 mL, 15.0 mmol, 1.1 equiv) in THF (150 mL) and HMPA (6.90 mL, 39.5 mmol, 2.9 equiv) at −78° C. under N2. The reaction mixture was stirred at this temperature for 20 min, and then quenched with 20% aqueous sulfuric acid (60 mL). The mixture was extracted with EtOAc and the combined organic layers were washed with saturated NaHCO₃ solution and then dried over Na2SO4. The sodium salts were filtered off and the filtrate was concentrated under reduced pressure. The residue was purified by silica gel column chromatography (PE/EA=3:1) affording the product as colorless oil (6.3 g, 97%).

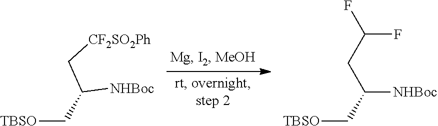

### Step 2: tert-butyl (S)-(1-((tert-butyldimethylsilyl)oxy)-4,4-difluorobutan-2-yl)carbamate

10 mL of methanol and I₂ (0.17 g, 0.69 mmol, 0.1 equiv) were added to Mg (5.02 g, 206.4 mmol, 30 equiv) stored under a nitrogen atmosphere. The mixture was cooled to 0° C. and a solution of tert-butyl (S)-(1-((tert-butyldimethylsilyl)oxy)-4,4-difluoro-4-(phenylsulfonyl)butan-2-yl)carbamate (3.3 g, 6.9 mmol, 1 equiv) in 30 mL methanol was added. The reaction mixture was allowed to warm to 20° C. and stirred at this temperature overnight. The reaction was quenched with saturated ammonium chloride solution and extracted with EtOAc. The combined organic layers were washed with brine, dried over anhydrous sodium sulfate, and filtered. The solvents were evaporated, and the residue was purified by silica gel column chromatography (PE/EA=7:1) affording the product as colorless oil (1.47 g, 63%).

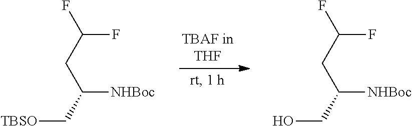 [SMILES: CC(C)(C)OC(=O)N[C@H](CO[Si](C)(C)C(C)(C)C)CC(F)F ; CC(C)(C)OC(=O)N[C@H](CO)CC(F)F]

### Step 3: tert-butyl (S)-(4,4-difluoro-1-hydroxybutan-2-yl)carbamate

To a solution of tert-butyl (S)-(1-((tert-butyldimethylsilyl)oxy)-4,4-difluorobutan-2-yl)carbamate (700 mg, 2.06 mmol, 1 equiv) in THF (7 mL) was added TBAF (1.65 mL, 1.65 mmol, 0.8 equiv) dropwise at 0° C. The resulting mixture was stirred for 1 h at room temperature and then concentrated under reduced pressure. The residue was purified by silica gel column chromatography (PE/EA=1:1) yielding the product as white solid (430 mg, 93%).

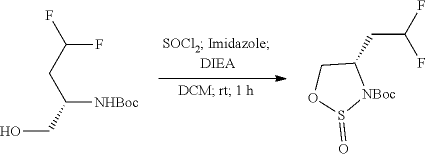 [SMILES: CC(C)(C)OC(=O)N[C@H](CO)CC(F)F ; CC(C)(C)OC(=O)N1[C@@H](CC(F)F)COS1=O]

### Step 4. tert-butyl (4S)-4-(2,2-difluoroethyl)-1,2,3-oxathiazolidine-3-carboxylate 2-oxide

A portion of SOCl₂ (0.21 mL, 2.86 mmol, 1.5 equiv) followed by DIEA (0.67 mL, 3.82 mmol, 2 equiv) were added dropwise at 0° C. to a solution of imidazole (520 mg, 7.64 mmol, 4 equiv) in DCM (13 mL). (S)-(4,4-difluoro-1-hydroxybutan-2-yl)carbamate (430 mg, 1.91 mmol, 1 equiv) dissolved in DCM (2 mL) was added dropwise at 0° C. and the resulting mixture was stirred for 1 h at room temperature. The mixture was washed with saturated NaHCO₃ solution and evaporated affording the product as yellow oil (500 mg, 97%).

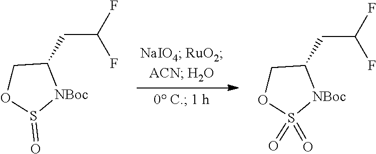 [SMILES: CC(C)(C)OC(=O)N1[C@@H](CC(F)F)COS1=O ; CC(C)(C)OC(=O)N1[C@@H](CC(F)F)COS1(=O)=O]

### Step 5: tert-butyl (S)-4-(2,2-difluoroethyl)-1,2,3-oxathiazolidine-3-carboxylate (AA-13)

To a solution of tert-butyl (4S)-4-(2,2-difluoroethyl)-1,2,3-oxathiazolidine-3-carboxylate 2-oxide (500 mg, 1.84 mmol, 1 equiv) in ACN (6.5 mL) and water (3.5 mL) were added NaIO₄ (591 mg, 2.76 mmol, 1.5 equiv) and ruthenium(iv) oxide hydrate (13.9 mg, 0.092 mmol, 0.05 equiv) at 0° C. The resulting mixture was stirred for 1 h at 0° C., and then filtered. The filtrate was extracted with EtOAc and the combined organic layers were washed with brine (20 mL) and dried over anhydrous Na₂SO₄. The solvents were removed under reduced pressure and the residue was purified by silica gel column chromatography (PE/EA=2:1). White solid (310 mg, 59%).

Reference for tert-butyl (S)-4-phenyl-1,2,3-oxathiazolidine-3-carboxylate 2,2-dioxide (AA-14): James, T.; Simpson, I.; Grant, J. A.; Sridharan, V.; Nelson, A. Organic Letters, 2013, 15, 6094-6097.

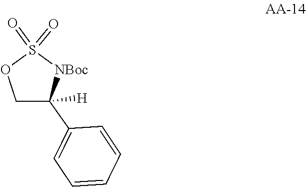 [SMILES: CC(C)(C)OC(=O)N1[C@@H](c2ccccc2)COS1(=O)=O]

### Synthesis of tert-butyl (R)-4-((R)-1-fluoroethyl)-1,2,3-oxathiazolidine-3-carboxylate 2,2-dioxide (AA-15)

### Step 1. Synthesis of tert-butyl ((2R,3S)-1,3-dihydroxybutan-2-yl)carbamate

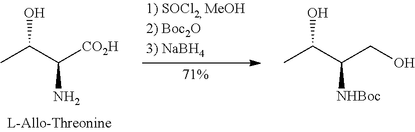

To a suspension of L-allothreonine (22.00 g, 97.00% Wt, 1 Eq, 179.1 mmol) in MeOH (500.00 mL) at 0° C., thionyl chloride (85.25 g, 52.30 mL, 4 Eq, 716.6 mmol) was added in a slow stream. The reaction mixture was allowed to warm to rt and refluxed for 3 h and concentrated in vacuo. The crude material was diluted with DCM (500.00 mL) and Boc₂O (39.10 g, 41.2 mL, 1 Eq, 179.1 mmol) was added followed by triethylamine (54.38 g, 74.9 mL, 3 Eq, 537.4 mmol). The reaction mixture was stirred at rt for 90 minutes, washed with water twice and also brine, dried over sodium sulfate, filtered and concentrated in vacuo to afford methyl (tert-butoxycarbonyl)-L-allothreoninate (39.140 g, 167.80 mmol, 93.66%). To a solution of methyl (tert-butoxycarbonyl)-L-allothreoninate (39.140 g, 1 Eq, 167.80 mmol) in EtOH (650.00 mL) at 0° C. was added NaBH4 (12.70 g, 2 Eq, 335.59 mmol). The reaction mixture was stirred at 0° C. overnight.

The reaction was quenched with water (400 mL) and diluted with DCM (500 mL). The mixture was stirred vigorously for 5 minutes. The phases were separated and the water layer was thoroughly extracted with DCM/EtOH 10% three times (3×750 mL). The combined organic layers were washed with brine (500 mL) dried over sodium sulfate, filtered and concentrated in vacuo to afford tert-butyl ((2R,3S)-1,3-dihydroxybutan-2-yl)carbamate (26.11 g, 127.2 mmol, 75.81%).

¹H NMR (299 MHz, CDCl₃) δ 5.57-5.13 (m, 1H), 4.10-3.93 (m, 2H), 3.87-3.66 (m, 1H), 3.51 (s, 1H), 2.62 (s, 2H), 1.47 (s, 9H), 1.31 (d, J=6.4 Hz, 3H).

### Step 2, Synthesis of Tert-butyl ((2R,3S)-1-(benzyloxy)-3-hydroxybutan-2-yl)carbamate

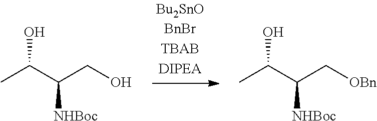 [SMILES: C[C@H](O)[C@@H](CO)NC(=O)OC(C)(C)C ; C[C@H](O)[C@@H](COCc1ccccc1)NC(=O)OC(C)(C)C]

A mixture of dibutyltin oxide (2.814 g, 0.1 Eq, 11.30 mmol), tetrabutylammonium bromide (10.93 g, 0.30 Eq, 33.91 mmol), DIPEA (58.44 g, 78.8 mL, 4 Eq, 452.1 mmol) and benzyl bromide (77.33 g, 53.78 mL, 4 Eq, 452.1 mmol) and tert-butyl ((2R,3S)-1,3-dihydroxybutan-2-yl)carbamate (23.2 g, 113 mmol) was stirred neat at 76° C. for 18 h under vigorous stirring. The reaction was cooled to room temperature and diluted with EtOAc and water. The water layer was extracted twice with EtOAc. The combined organic layers were washed with brine, dried over sodium sulfate, filtered and concentrated in vacuo. Purification by column chromatography (H/EA, 70/30) afforded tert-butyl ((2R,3S)-1-(benzyloxy)-3-hydroxybutan-2-yl)carbamate.

¹H NMR (299 MHz, CDCl₃) δ 7.51-7.31 (m, 5H), 5.28 (s, 1H), 4.64-4.48 (m, 2H), 3.96-3.77 (m, 2H), 3.73-3.56 (m, 2H), 2.92 (d, J=8.2 Hz, 1H), 1.47 (s, 9H), 1.25 (d, 3H).

### Step 3. Synthesis of tert-butyl (4R,5S)-4-((benzyloxy)methyl)-5-methyl-1,2,3-oxathiazolidine-3-carboxylate 2,2-dioxide

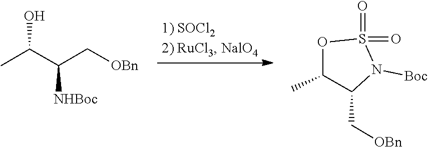 [SMILES: C[C@H](O)[C@@H](COCc1ccccc1)NC(=O)OC(C)(C)C ; C[C@@H]1OS(=O)(=O)N(C(=O)OC(C)(C)C)[C@@H]1COCc1ccccc1]

To a solution of imidazole (18.81 g, 4 Eq, 276.3 mmol) and triethylamine (17.47 g, 24.1 mL, 2.5 Eq, 172.7 mmol) in DCM (550.00 mL) at −60° C. was added thionyl chloride (9.448 g, 5.796 mL, 1.15 Eq, 79.42 mmol) dropwise keeping the reaction mixture below −55° C. Next, a solution of tert-butyl ((2R,3S)-1-(benzyloxy)-3-hydroxybutan-2-yl)carbamate (20.40 g, 1 Eq, 69.06 mmol) in DCM (200.00 mL) was added dropwise keeping the reaction mixture below −55° C. and the reaction mixture was allowed to warm to rt slowly. The reaction was quenched with 0.5N HCl (1 L). The phases were separated and the acidic layer was extracted with DCM (2×500 mL). The combined organic layers were washed with brine (300 mL), dried over sodium sulfate, filtered and concentrated in vacuo. To a solution of the crude material in acetonitrile (200.00 mL) at 0° C. was added sodium periodate (16.99 g, 1.15 Eq, 79.42 mmol) followed by ruthenium trichloride (1.433 g, 0.1 Eq, 6.906 mmol) and water (200.00 mL). The mixture was stirred at 0° C. for 45 minutes and monitored by NMR for full conversion. The reaction was diluted with water (200 mL) and TBME (300 mL), and filtered through a pad of celite. The water layer was extracted with TBME (3×400 mL). The combined organic layers were washed with brine (300 mL), dried over sodium sulfate, filtered and concentrated in vacuo. The crude material was purified over a short plug of silica, eluting with TBME to afford tert-butyl (4R,5S)-4-((benzyloxy)methyl)-5-methyl-1,2,3-oxathiazolidine-3-carboxylate 2,2-dioxide (24.38 g, 68.21 mmol, 98.77%).

¹H NMR (299 MHz, CDCl₃) δ 7.48-7.31 (m, 5H), 5.24-4.97 (m, 1H), 4.58 (s, 2H), 4.39-4.26 (m, 1H), 3.85 (dd, J=10.2, 7.4 Hz, 1H), 3.67 (dd, J=10.3, 2.9 Hz, 1H), 1.81-1.40 (m, 12H).

### Step 4. Synthesis of tert-butyl ((2R,3R)-1-(benzyloxy)-3-fluorobutan-2-yl)carbamate

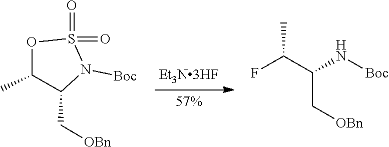 [SMILES: C[C@@H]1OS(=O)(=O)N(C(=O)OC(C)(C)C)[C@@H]1COCc1ccccc1 ; C[C@@H](F)[C@@H](COCc1ccccc1)NC(=O)OC(C)(C)C]

To a solution of tert-butyl (4R,5S)-4-((benzyloxy)methyl)-5-methyl-1,2,3-oxathiazolidine-3-carboxylate 2,2-dioxide (16.30 g, 1 Eq, 45.60 mmol) in toluene (250.00 mL) was added triethylamine trihydrofluoride (47.79 g, 48.3 mL, 6.5 Eq, 296.4 mmol) and the reaction was refluxed and stopped after 2.5 hours. The reaction was cooled to rt and the phases were separated. The bottom layer extracted twice with toluene (2×250 mL). The combined organic layers were washed with saturated sodium bicarbonate and brine, dried over sodium sulfate, filtered and concentrated in vacuo to afford 9 g of crude material. The crude was purified by column chromatography (Heptane/EA, 90/10) to afford tert-butyl ((2R,3R)-1-(benzyloxy)-3-fluorobutan-2-yl)carbamate (6.360 g, 21.39 mmol, 46.90%). Additional material could be obtained as follows: further elution of the chromatography column afforded des-boc sulfamidate (Heptane/EA, 65/35). The initial aqueous layer was diluted with EtOAc (250 mL) and basified with a saturated sodium bicarbonate solution. The water layer was extracted twice with EtOAc (2×500 mL) and the combined organic layers washed with saturated sodium bicarbonate and brine, dried over sodium sulfate, filtered and concentrated in vacuo to afford 3.8 g of a mixture of compounds. The mixture was reprotected with Boc₂O and 1.57 g of additional desired product was isolated after column chromatography together with 1.7 g of de-boc sulfamidate which gave 2.36 g of boc-protected sulfamidate that was reconverted again to the desired product as described above. In total, 11.5 g of material were obtained starting from 24.4 g of starting material (57% yield).

¹H NMR (299 MHz, CDCl₃) δ 7.44-7.29 (m, 5H), 5.07-4.83 (m, 1H), 4.76 (d, J=9.3 Hz, 1H), 4.55 (d, J=2.0 Hz, 2H), 3.97-3.76 (m, 1H), 3.61-3.38 (m, 2H), 1.47 (s, 9H), 1.37 (dd, J=24.4, 6.4 Hz, 3H).

### Step 5. Tert-butyl ((2R,3R)-3-fluoro-1-hydroxybutan-2-yl)carbamate

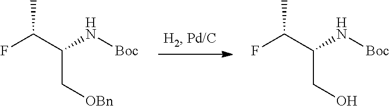 [SMILES: C[C@@H](F)[C@@H](COCc1ccccc1)NC(=O)OC(C)(C)C ; C[C@@H](F)[C@@H](CO)NC(=O)OC(C)(C)C]

To a solution of tert-butyl ((2R,3R)-1-(benzyloxy)-3-fluorobutan-2-yl)carbamate (11.50 g, 1 Eq, 38.67 mmol) in EtOAc (250.00 mL) was added PdOH₂ (1.20 g, 0.221 Eq, 8.55 mmol) and the reaction mixture was stirred under an Hydrogen gas overnight. The reaction was deemed complete by NMR. The mixture was filtered over celite and the cake was washed twice with EtOAc (2×200 mL) and concentrated in vacuo. The crude material was purified over column chromatography (Heptanes/EA, 60/40) to afford a total 5.80 g of tert-butyl ((2R,3R)-3-fluoro-1-hydroxybutan-2-yl)carbamate

¹H NMR (299 MHz, CDCl₃) δ 5.24-4.56 (m, 2H), 3.90-3.48 (m, 3H), 2.09 (d, J=14.2 Hz, 1H), 1.48 (s, 9H), 1.40 (dd, J=24.7, 6.4 Hz, 3H).

### Step 6. Synthesis of tert-butyl (R)-4-((R)-1-fluoroethyl)-1,2,3-oxathiazolidine-3-carboxylate 2,2-dioxide

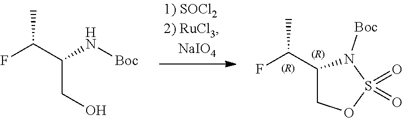

To a solution of imidazole (7.62 g, 4 Eq, 112 mmol), triethylamine (7.08 g, 9.75 mL, 2.5 Eq, 70.0 mmol) in DCM (160 mL) at −60° C. was added dropwise thionyl chloride (3.99 g, 2.45 mL, 1.2 Eq, 33.6 mmol) followed by tert-butyl ((2R,3R)-3-fluoro-1-hydroxybutan-2-yl)carbamate (5.80 g, 1 Eq, 28.0 mmol) in DCM (60 mL) keeping the reaction mixture below −55° C. The turbid mixture was allowed to warm to rt slowly and stirred for 30 minutes. The reaction was quenched with 0.5N HCl (500 mL). The phases were separated and the water layer was extracted with DCM (2×200 mL). The combined organic layers were washed with brine (250 mL), dried over sodium sulfate, filtered, and concentrated in vacuo.

To a solution of the crude material in acetonitrile (60 mL) at 0° C. was added sodium periodate (6.88 g, 1.15 Eq, 32.2 mmol) followed by ruthenium trichloride (580 mg, 187 μL, 0.1 Eq, 2.80 mmol) and water (60 mL). The mixture was stirred at 0° C. for 45 minutes. Full conversion was noted by NMR. The reaction was diluted with water (200 mL) and TBME (300 mL), filtered over celite. The water layer was extracted with TBME (3×400 mL). The combined organic layers were washed with brine (300 mL), dried over sodium sulfate, filtered and concentrated in vacuo. Crude material purified by column chromatography (500 mL silica, Hept/EtOAc, 80/20 to 75/25) to afford tert-butyl (R)-4-((R)-1-fluoroethyl)-1,2,3-oxathiazolidine-3-carboxylate 2,2-dioxide (5.46 g, 20.3 mmol, 72.5%) as a white solid.

¹H NMR (299 MHz, CDCl₃ δ 5.23-4.88 (m, 1H), 4.69-4.64 (m, 2H), 4.61-4.51 (m, 1H), 1.58 (s, 9H), 1.48 (dd, J=24.4, 6.5 Hz, 3H).

### Synthesis of tert-butyl (4S)-4-(cyclopropoxymethyl)-2,2-dioxo-1,2lambda6,3-oxathiazolidine-3-carboxylate (AA-16)

### Step 1.

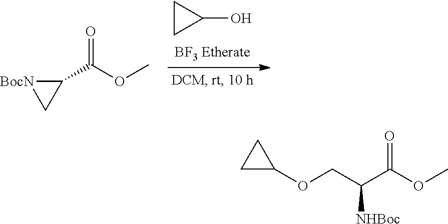

To a stirred mixture of 1-tert-butyl 2-methyl (2S)-aziridine-1,2-dicarboxylate vanadium (500 mg, 1.983 mmol, 1 equiv) and cyclopropanol (426.10 mg, 7.337 mmol, 3.7 equiv) in DCM (5 mL) was added BF₃·Et₂O (28.14 mg, 0.198 mmol, 0.1 equiv) dropwise at 0° C. under nitrogen atmosphere. The resulting mixture was stirred for overnight at room temperature under nitrogen atmosphere. The resulting mixture was diluted with water (5 mL). The resulting mixture was extracted with CH₂Cl₂ (3×50 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 30% to 100% gradient in 15 min; detector, UV 190 nm. This resulted in methyl (2S)-2-[(tert-butoxycarbonyl)amino]-3-cyclopropoxypropanoate (200 mg, 38.90%) as a yellow oil.

### Step 2.

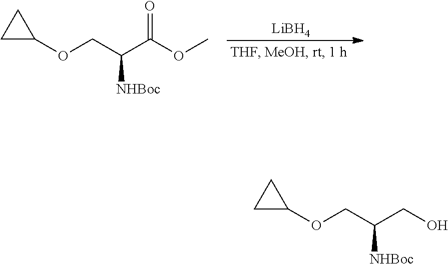 [SMILES: COC(=O)[C@H](COC1CC1)NC(=O)OC(C)(C)C ; "LiBH4" ; CC(C)(C)OC(=O)N[C@H](CO)COC1CC1]

To a stirred solution of methyl (2S)-2-[(tert-butoxycarbonyl)amino]-3-cyclopropoxypropanoate (1.8 g, 6.942 mmol, 1 equiv) in tetrahydrofuran (20 mL) was added LiBH₄ (453.57 mg, 20.82 mmol, 3 equiv) dropwise at 0° C. under nitrogen atmosphere. The resulting mixture was stirred for 1 h at room temperature under nitrogen atmosphere. The reaction was quenched by the addition of water/Ice (10 mL) at room temperature. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (4:1) to afford tert-butyl N-[(2R)-1-cyclopropoxy-3-hydroxypropan-2-yl]carbamate (1.3 g, 80.97%) as a colorless oil.

### Step 3.

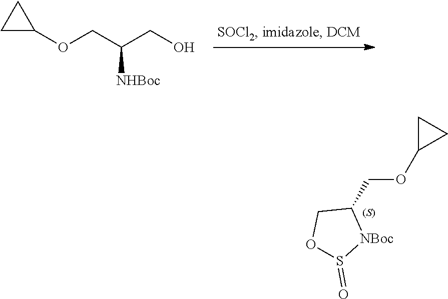

To a solution of Imidazole (1.05 g, 15.392 mmol, 4 equiv) in DCM (30 mL) were added SOCl₂ (0.42 mL, 5.772 mmol, 1.5 equiv) followed by DIEA (1.34 mL, 7.696 mmol, 2 equiv) dropwise at 0° C. To the above mixture was added tert-butyl N-[(2R)-1-cyclopropoxy-3-hydroxypropan-2-yl]carbamate (890 mg, 3.848 mmol, 1 equiv) dissolved in DCM (5 mL) dropwise at 0° C. The resulting mixture was stirred for additional 1 h at room temperature. The resulting mixture was washed with 3×30 mL of conc. HCl (0.5 M). The resulting mixture was concentrated under vacuum to afford tert-butyl (4S)-4-(cyclopropoxymethyl)-2-oxo-1,2lambda4,3-oxathiazolidine-3-carboxylate (1.06 g, 99.33%) as a yellow solid.

### Step 4.

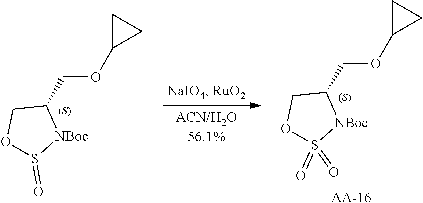

To a solution of tert-butyl (4S)-4-(cyclopropoxymethyl)-2-oxo-1,2lambda4,3-oxathiazolidine-3-carboxylate (1.06 g, 3.822 mmol, 1 equiv) in MeCN (13 mL) and H₂O (7 mL) was added NaIO₄ (0.98 g, 4.586 mmol, 1.2 equiv) and ruthenium(iv) oxide hydrate (11.55 mg, 0.076 mmol, 0.02 equiv) at 0° C. The resulting mixture was stirred for 1 h at 0° C. The resulting mixture was filtered through a Celite pad. The filtrate was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (3:1) to afford tert-butyl (4S)-4-(cyclopropoxymethyl)-2,2-dioxo-1,2lambda6,3-oxathiazolidine-3-carboxylate (AA-16, 0.63 g, 56.19%) as a colorless oil.

Reference for 3-(tert-butyl) 4-methyl (S)-1,2,3-oxathiazolidine-3,4-dicarboxylate 2,2-dioxide (AA-17): Baig, N.; Chandrakala, R. N.; Sudhir, V. S.; Chandresekaran, S.; J. Org. Chem. 2010, 75, 2910-2921.

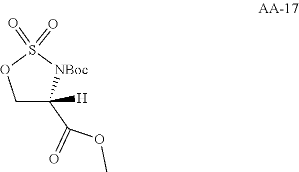 [SMILES: COC(=O)[C@@H]1COS(=O)(=O)N1C(=O)OC(C)(C)C]

Reference for tert-butyl (S)-4-ethyl-1,2,3-oxathiazolidine-3-carboxylate 2,2-dioxide (AA-18): Baig, N. Kanimozhi, C. K.; Sudhir, V. S.; Chandrasekran, S. Synlett 2009, 8, 1227-1232.

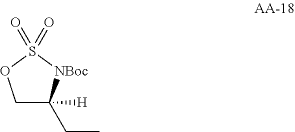 [SMILES: CC[C@H]1COS(=O)(=O)N1C(=O)OC(C)(C)C]

Synthesis of tert-butyl 4-((1-fluorocyclopropyl)methyl)-1,2,3-oxathiazolidine-3-carboxylate 2,2-dioxide (AA-19) was performed according the general method starting from tert-butyl (1-(1-fluorocyclopropyl)-3-hydroxypropan-2-yl)carbamate.

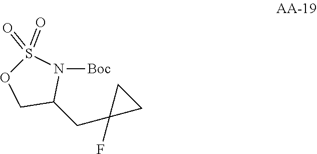 [SMILES: CC(C)(C)OC(=O)N1C(CC2(F)CC2)COS1(=O)=O]

### Synthesis of tert-butyl (1-(1-fluorocyclopropyl)-3-hydroxypropan-2-yl)carbamate

### Step-1: Synthesis of (1-fluorocyclopropyl)methanol

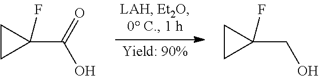 [SMILES: O=C(O)C1(F)CC1 ; OCC1(F)CC1]

To a stirred solution of 1-fluorocyclopropane-1-carboxylic acid (3.0 g, 29.0 mmol) in diethyl ether (30 mL) was added LAH (34 mL, 1 molar, 34.0 mmol) at 0° C. The resulting reaction mixture was stirred at 0° C. for 1 h. Reaction was quenched with saturated Na₂SO₄ solution at 0° C. and stirred for 30 minutes at 0° C. The reaction mixture was filtered through celite pad and the celite pad was washed with diethyl ether. The filtrate was dried over Na₂SO₄. Solvent was evaporated by downward distillation without vacuum at bath temperature 60° C. to afford (1-fluorocyclopropyl)methanol (4 g, 90% yield) as colourless liquid.

### Step-2: Synthesis of (1-fluorocyclopropyl)methyl 4-methylbenzenesulfonate

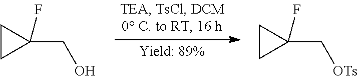 [SMILES: OCC1(F)CC1 ; Cc1ccc(S(=O)(=O)OCC2(F)CC2)cc1]

To a stirred solution (1-fluorocyclopropyl)methanol (15 g, 0.15 mol) in DCM (500 mL) was added triethylamine (53 mL, 0.38 mol) at 0° C. followed by tosyl-Cl (44 g, 0.23 mol). The resulting reaction mixture was stirred for 16 h at 28° C. Reaction mixture was diluted with DCM (500 mL), organic layer was washed with saturated NaHCO₃ solution (2×400 mL), water (100 mL) and brine solution (100 mL). Organic layer was dried over Na₂SO₄, filtered and concentrated under reduced pressure to afford crude product. The crude compound was purified via flash column chromatography using silica gel (100-200, 120 g cartridge) and compound was eluted with a gradient of 10-60% EtOAc/petroleum ether. Pure fractions were combined and concentrated under reduced pressure to afford (1-fluorocyclopropyl)methyl 4-methylbenzenesulfonate (33 g, 89% yield) as an off-white solid.

### Step-3: Synthesis of tert-butyl 2-((diphenylmethylene)amino)-3-(1-fluorocyclopropyl)propanoate

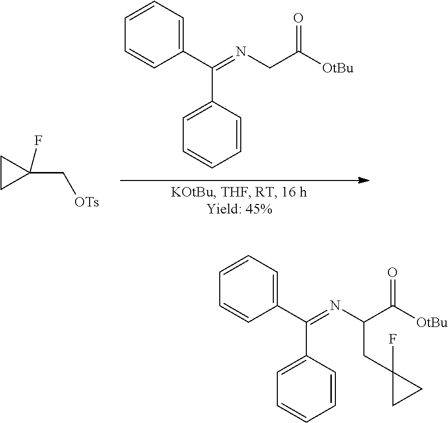 [SMILES: Cc1ccc(S(=O)(=O)OCC2(F)CC2)cc1 ; CC(C)(C)OC(=O)CN=C(c1ccccc1)c1ccccc1 ; CC(C)(C)OC(=O)C(CC1(F)CC1)N=C(c1ccccc1)c1ccccc1]

To a stirred solution of tert-butyl 2-((diphenylmethylene)amino)acetate (40.0 g, 134 mmol) and (1-fluorocyclopropyl)methyl 4-methylbenzenesulfonate (40.7 g, 158 mmol) in THF (800 mL) was added KOtBu (in THF) (161 mL, 1 molar, 161 mmol) at room temperature. The resulting reaction mixture was stirred for 16 h at room temperature. Reaction mixture was diluted with water (300 mL) and extracted with ethyl acetate (3×500 mL). Organic layer was dried over anhydrous sodium sulfate, filtered and concentrated under reduced pressure to afford tert-butyl 2-((diphenylmethylene)amino)-3-(1-fluorocyclopropyl)propanoate (60 g, 45% yield) as a brown color gummy compound. LCMS: m/z 368.23 [M+H]⁺; t_{R}=5.30 min.

### Step-4: Synthesis of tert-butyl 2-((tert-butoxycarbonyl)amino)-3-(1-fluorocyclopropyl)propanoate

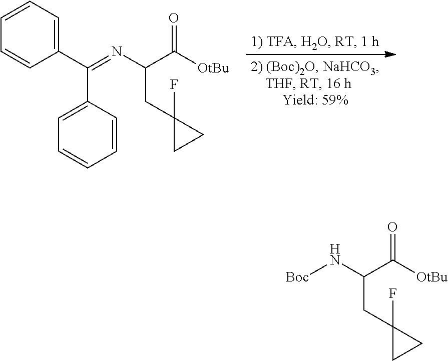 [SMILES: CC(C)(C)OC(=O)C(CC1(F)CC1)N=C(c1ccccc1)c1ccccc1 ; CC(C)(C)OC(=O)NC(CC1(F)CC1)C(=O)OC(C)(C)C]

To a stirred solution of tert-butyl 2-((diphenylmethylene)amino)-3-(1-fluorocyclopropyl)propanoate (60 g, 61.0 mmol) in water (600 mL) was added TFA (40 mL, 0.51 mol) at 25° C. and the reaction mixture was stirred for 1 hour. Reaction mixture pH was adjusted to ˜8 with solid sodium bicarbonate and then added THF (300 mL) at room temperature. After that added (Boc)₂O (21 mL, 91.0 mmol) at 25° C. and the reaction mixture was stirred for 16 hours. Reaction was diluted with water (1 L) and extracted with ethyl acetate (2×1 L). Combined organic layer was washed with water (2×500 mL), brine, dried over sodium sulfate and concentrated under reduced pressure to afford crude material. Crude material was purified by flash chromatography using (100-200 mesh silica gel) and compound was eluted with a gradient of 0-15% ethyl acetate in petroleum ether. Pure fractions were concentrated under reduced pressure to afford tert-butyl 2-((tert-butoxycarbonyl)amino)-3-(1-fluorocyclopropyl)propanoate (12 g, 59% yield) as an off-white solid. ELSD: m/z 304.29 [M+H]⁺; t_{R}=3.51 min.

### Step-5: Synthesis of tert-butyl (1-(1-fluorocyclopropyl)-3-hydroxypropan-2-yl)carbamate

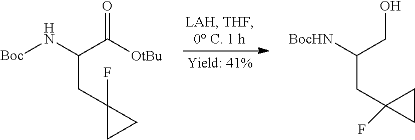 [SMILES: CC(C)(C)OC(=O)NC(CC1(F)CC1)C(=O)OC(C)(C)C ; CC(C)(C)OC(=O)NC(CO)CC1(F)CC1]

To a stirred solution of tert-butyl 2-((tert-butoxycarbonyl)amino)-3-(1-fluorocyclopropyl)propanoate (12 g, 36.0 mmol) in THF (250 mL) was added LiAlH₄ (19 mL, 46.0 mmol) at 0° C. and stirred at same temperature for 1 hour. Reaction was quenched by the addition of 50 mL saturated sodium sulfate solution, the resulting reaction mixture was diluted with ethyl acetate and filtered through celite pad. Filtrate was concentrated under reduced pressure to afford crude material. Crude material was purified by flash chromatography using 100-200 mesh silica gel and compound was eluted with a gradient of 20% ethyl acetate in petroleum ether. Pure fractions were concentrated under reduced pressure to afford tert-butyl (1-(1-fluorocyclopropyl)-3-hydroxypropan-2-yl)carbamate (3.8 g, 41% yield) as a white solid. LCMS: m/z 368.24 [M+H]⁺; t_{R}=1.20 min.

### Synthesis of tert-butyl 4-(oxetan-3-ylmethyl)-1,2,3-oxathiazolidine-3-carboxylate 2,2-dioxide (AA-20)

### Step-1: Synthesis of oxetan-3-ylmethyl 4-methylbenzenesulfonate

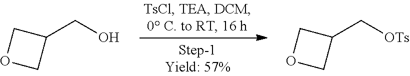 [SMILES: OCC1COC1 ; Cc1ccc(S(=O)(=O)OCC2COC2)cc1]

To a stirred solution of oxetan-3-ylmethanol (25 g, 0.28 mol) in DCM (500 mL) was added TEA (125 mL, 879 mmol) at 0° C. and stirred the reaction mixture at same temperature for 30 minutes. Then TsCl (76 g, 0.39 mol) was added to the reaction mixture at at 0° C. and stirred at 25° C. for 16 hours. Progress of the reaction was monitored by TLC and LCMS. On completion, the reaction mixture was diluted with water (500 mL) and separated the organic layer. Organic layer was washed with water (2×200 mL) and brine, dried over sodium sulphate and concentrated under reduced pressure to afford crude material. The crude material was triturated with petroleum ether (2×100 mL) and dried under reduced pressure to afford oxetan-3-ylmethyl 4-methylbenzenesulfonate (50 g, 57% yield) as a light brown thick liquid. LCMS: m/z 243.18 [M+H]⁺; t_{R}=1.68 min.

### Step-2: Synthesis of tert-butyl 2-((diphenylmethylene)amino)-3-(oxetan-3-yl)propanoate

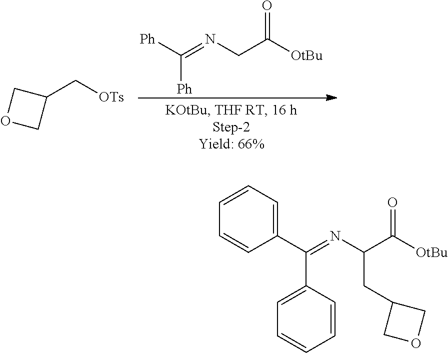 [SMILES: Cc1ccc(S(=O)(=O)OCC2COC2)cc1 ; CC(C)(C)OC(=O)CN=C(c1ccccc1)c1ccccc1 ; CC(C)(C)OC(=O)C(CC1COC1)N=C(c1ccccc1)c1ccccc1]

To a stirred solution of tert-butyl 2-((diphenylmethylene)amino)acetate (50 g, 0.17 mol), oxetan-3-ylmethyl 4-methylbenzenesulfonate (50 g, 0.16 mol) in THE (1 L) was added potassium tert-butoxide in THE (190 mL, 1 M, 190 mmol) slowly at room temperature and stirred the reaction mixture at 25° C. for 16 hours. On completion, reaction mixture was diluted with water (5 L) and extracted with ethyl acetate (2×2 L). Combined organic layer was washed with water (2×1 L), brine, dried over sodium sulphate and concentrated under reduced pressure to afford tert-butyl 2-((diphenylmethylene)amino)-3-(oxetan-3-yl)propanoate (100 g, 66% yield) crude as a brown light brown color thick liquid. LCMS: m/z 366.27 [M+H]⁺; t_{R}=4.85 min.

### Step-3: Synthesis of tert-butyl 2-((tert-butoxycarbonyl)amino)-3-(oxetan-3-yl)propanoate

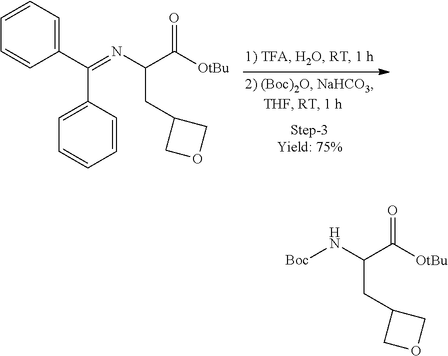 [SMILES: CC(C)(C)OC(=O)C(CC1COC1)N=C(c1ccccc1)c1ccccc1 ; CC(C)(C)OC(=O)NC(CC1COC1)C(=O)OC(C)(C)C]

To a stirred solution of tert-butyl 2-((diphenylmethylene)amino)-3-(oxetan-3-yl)propanoate (100 g, 109 mmol) in water (600 mL) was added trifluoroacetic acid (43.0 mL, 547 mmol) at room temperature and stirred the reaction mixture at 25° C. for 1 hour. Progress of the reaction was monitored by TLC. Then the reaction mixture pH was adjusted to ˜8 with sodium bicarbonate and added THE (300 mL) followed by (Boc)₂O (38.5 mL, 164 mmol) at 25° C. and stirred at same temperature for 1 hour. Progress of the reaction was monitored by TLC and LCMS. On completion, reaction was diluted with water (500 mL) and extracted with ethyl acetate (2×1000 mL). Combined organic layer was washed with water (2×500 mL) and brine. Dried the organic layer over sodium sulphate and concentrated under reduced pressure to afford crude material. Crude material was purified by flash chromatography using 100-200 mesh silica gel and compound was eluted with a gradient of 30% ethyl acetate in petroleum ether. Pure fractions were combined (determined by TLC) and concentrated under reduced pressure to afford tert-butyl 2-((tert-butoxycarbonyl)amino)-3-(oxetan-3-yl)propanoate (25 g, 75% yield) as a colorless thick liquid. LCMS: m/z 302.42 [M+H]⁺; t_{R}=1.88 min.

### Step-4: Synthesis of tert-butyl (1-hydroxy-3-(oxetan-3-yl)propan-2-yl)carbamate

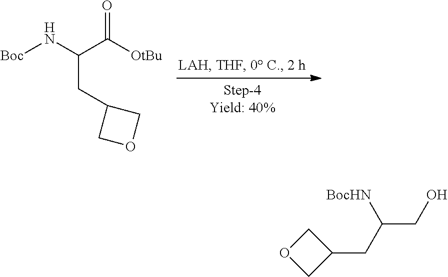 [SMILES: CC(C)(C)OC(=O)NC(CC1COC1)C(=O)OC(C)(C)C ; CC(C)(C)OC(=O)NC(CO)CC1COC1]

To a stirred solution of tert-butyl 2-((tert-butoxycarbonyl)amino)-3-(oxetan-3-yl)propanoate (10 g, 33 mmol) in THF (100 mL) was added LAH in THF (18 mL, 2.4 M, 43 mmol) at 0° C. and stirred the reaction mixture at same temperature for 2 hours. Reaction was monitored by TLC. On completion, reaction mixture was quenched with saturated Na2SO4 solution at 0° C. and stirred for 10 minutes. The reaction mixture was filtered through celite and celite pad was washed with 10% methanol in dichloromethane. The filtrate was dried over anhydrous Na₂SO₄, filtered and concentrated under reduced pressure to afford crude product. The crude was purified by flash column chromatography using YMC silica gel (40-63 m, 60 Å, 120 g cartridge) and compound was eluted with a gradient of 0-10% methanol in dichloromethane. Pure fractions were concentrated under reduced pressure to afford tert-butyl (1-hydroxy-3-(oxetan-3-yl)propan-2-yl)carbamate (3 g, 40% yield). LCMS: m/z 232.34 [M+H]⁺; t_{R}=1.27 min.

Compound AA-20 (tert-butyl 4-(oxetan-3-ylmethyl)-1,2,3-oxathiazolidine-3-carboxylate 2,2-dioxide) was then made from tert-butyl (1-hydroxy-3-(oxetan-3-yl)propan-2-yl)carbamate according to the general method.

### Synthesis of tert-butyl 4-((3-fluorooxetan-3-yl)methyl)-1,2,3-oxathiazolidine-3-carboxylate 2,2-dioxide (AA-21)

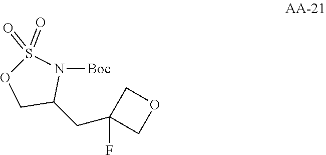 [SMILES: CC(C)(C)OC(=O)N1C(CC2(F)COC2)COS1(=O)=O]

### Step-1: Synthesis of tert-butyl 2-((diphenylmethylene)amino)-3-(3-fluorooxetan-3-yl)propanoate

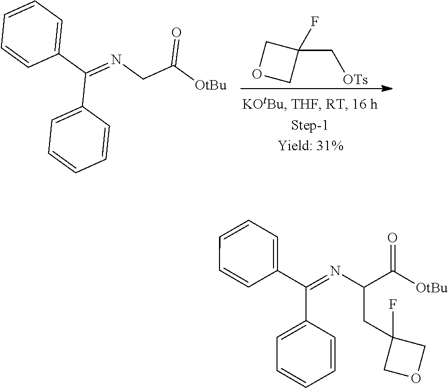 [SMILES: CC(C)(C)OC(=O)CN=C(c1ccccc1)c1ccccc1 ; Cc1ccc(S(=O)(=O)OCC2(F)COC2)cc1 ; CC(C)(C)OC(=O)C(CC1(F)COC1)N=C(c1ccccc1)c1ccccc1]

To a stirred solution of tert-butyl 2-((diphenylmethylene)amino)acetate (10 g, 34 mmol) and (3-fluorooxetan-3-yl)methyl 4-methylbenzenesulfonate (11 g, 40 mmol) in THE (20 mL) was added KO^{t}Bu (80 mL, 1M, 54 mmol) at room temperature. The resulting reaction mixture was stirred at same temperature for 16 h. Reaction was monitored by LCMS & TLC. Reaction mixture was diluted with water (250 mL) and extracted with ethyl acetate (2×300 mL). Organic layer was dried over anhydrous sodium sulphate, filtered and concentrated under reduced pressure to obtain the crude compound (12 g) as a brown solid. Crude material was triturated with n-pentane (100 mL) to afford tert-butyl 2-((diphenylmethylene)amino)-3-(3-fluorooxetan-3-yl)propanoate (12 g, 31% yield) as a light yellow solid. LCMS: m/z 384.55 [M+H]⁺; t_{R}=1.22 min.

### Step-2: Synthesis of tert-butyl 2-amino-3-(3-fluorooxetan-3-yl)propanoate

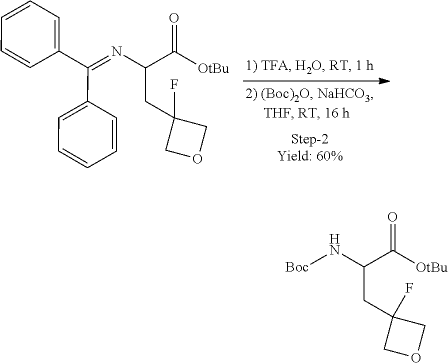 [SMILES: CC(C)(C)OC(=O)C(CC1(F)COC1)N=C(c1ccccc1)c1ccccc1 ; CC(C)(C)OC(=O)NC(CC1(F)COC1)C(=O)OC(C)(C)C]

To a stirred solution of tert-butyl 2-((diphenylmethylene)amino)-3-(3-fluorooxetan-3-yl)propanoate (12 g, 15 mmol) in water (150 mL) was added trifluoroacetic acid (14 mL, 74 mmol) at room temperature and stirred the reaction mixture at 25° C. for 1 hour. On completion, reaction mixture was basified with solid NaHCO₃ and then added THE (100 mL) at room temperature. After that (Boc)₂O (7.7 mL, 33 mmol) was added at RT and stirred for 16 h. On completion, reaction mixture was extracted with EtOAc (2×300 mL). The organic layer was dried over sodium sulphate and concentrated under reduced pressure obtained crude product (6 g). Crude material was purified by flash chromatography using 120 g silica gel cartridge and compound was eluted with a gradient of 14% EtOAc/hexane. Pure fractions were combined (determined by TLC) and concentrated under reduced pressure to afford tert-butyl 2-((tert-butoxycarbonyl)amino)-3-(3-fluorooxetan-3-yl)propanoate (4 g, 60% yield) as a light brown gum. LCMS: m/z 320.42 [M+H]⁺; t_{R}=1.94 min.

### Step-3: Synthesis of tert-butyl (1-(3-fluorooxetan-3-yl)-3-hydroxypropan-2-yl)carbamate

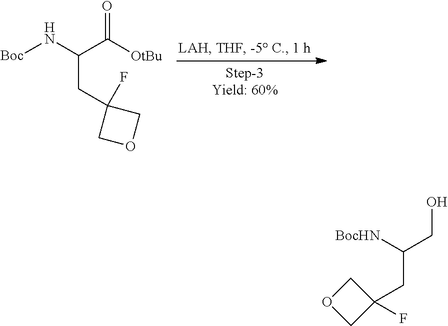 [SMILES: CC(C)(C)OC(=O)NC(CC1(F)COC1)C(=O)OC(C)(C)C ; CC(C)(C)OC(=O)NC(CO)CC1(F)COC1]

To a stirred solution of tert-butyl 2-((tert-butoxycarbonyl)amino)-3-(3-fluorooxetan-3-yl)propanoate (4 g, 0.01 mol) in THE (30 mL) was added LAH (20 mL, 2M, 0.03 mol) at −5° C. and stirred at same temperature for 1 hour. The reaction mixture was quenched with aqueous NH₄Cl (20 mL) and extracted with EtOAc (2×20 mL). The combined organic layer was dried over Na₂SO₄, filtered and concentrated under reduced pressure to obtain the crude compound as a brown liquid. The crude compound was purified through column chromatography (Silica gel 100-200 mesh; 50% EtOAc/pet ether as eluent). The collected pure fractions were concentrated under reduced pressure to afford tert-butyl (1-(3-fluorooxetan-3-yl)-3-hydroxypropan-2-yl)carbamate (1.2 g, 60% yield) as an off-white solid.

Compound AA-21 (tert-butyl 4-((3-fluorooxetan-3-yl)methyl)-1,2,3-oxathiazolidine-3-carboxylate 2,2-dioxide) was synthesized from tert-butyl (1-(3-fluorooxetan-3-yl)-3-hydroxypropan-2-yl)carbamate according to the general method.

### Synthesis of tert-butyl 4-(tetrahydrofuran-3-yl)-1,2,3-oxathiazolidine-3-carboxylate 2,2-dioxide (AA-22)

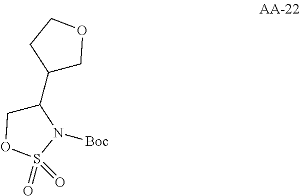 [SMILES: CC(C)(C)OC(=O)N1C(C2CCOC2)COS1(=O)=O]

Compound AA-22 was made from commercially available 2-amino-2-(tetrahydrofuran-3-yl)ethanol which was Boc protected on nitrogen and processed according to the general method.

Synthesis of tert-butyl (S)-4-((trifluoromethoxy)methyl)-1,2,3-oxathiazolidine-3-carboxylate 2,2-dioxide (AA-24) was performed according to the general method starting from tert-butyl (R)-(1-hydroxy-3-(trifluoromethoxy)propan-2-yl)carbamate (WO2021/087018).

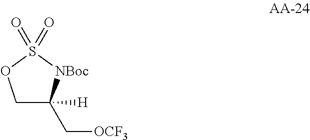 [SMILES: CC(C)(C)OC(=O)N1[C@@H](COC(F)(F)F)COS1(=O)=O]

Synthesis of tert-butyl (R)-4-((S)-1-((tert-butyldimethylsilyl)oxy)ethyl)-1,2,3-oxathiazolidine-3-carboxylate 2,2-dioxide (AA-25) was performed according to the general method starting from tert-butyl N-[(2R,3S)-3-[(tert-butyldimethylsilyl)oxy]-1-hydroxybutan-2-yl]carbamate

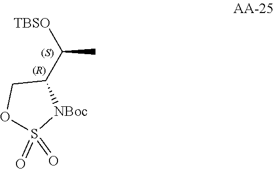

### Synthesis of tert-butyl N-[(2R,3S)-3-[(tert-butyldimethylsilyl)oxy]-1-hydroxybutan-2-yl]carbamate

### Step 1. Synthesis of methyl (2S,3S)-2-[(tert-butoxycarbonyl)amino]-3-hydroxybutanoate

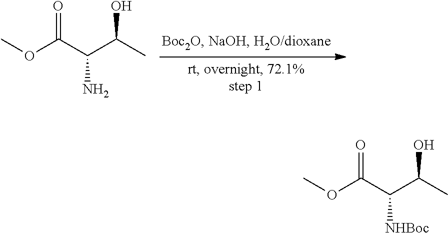

Into a 500 mL 3-necked round-bottom flask were added methyl (2S,3S)-2-amino-3-hydroxybutanoate (9.5 g, 71.350 mmol, 1 equiv), Boc₂O (18.69 g, 85.620 mmol, 1.2 equiv), NaOH (2.85 g, 71.350 mmol, 1 equiv), H₂O (113 mL) and dioxane (100 mL) at room temperature. The resulting mixture was stirred for overnight at room temperature. The resulting mixture was extracted with EtOAc (2×200 mL). The combined organic layers were washed with brine (200 mL), dried over anhydrous Na₂SO₄. After filtration, the filtrate was concentrated under reduced pressure. This resulted in methyl (2S,3S)-2-[(tert-butoxycarbonyl)amino]-3-hydroxybutanoate (12 g, 72.10%) as a colorless oil.

### Step 2. Synthesis of methyl (2S,3S)-2-[(tert-butoxycarbonyl)amino]-3-[(tert-butyldimethylsilyl)oxy]butanoate

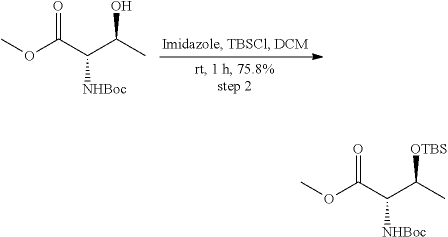 [SMILES: COC(=O)[C@@H](NC(=O)OC(C)(C)C)[C@H](C)O ; COC(=O)[C@@H](NC(=O)OC(C)(C)C)[C@H](C)O[Si](C)(C)C(C)(C)C]

Into a 500 mL 3-necked round-bottom flask were added methyl (2S,3S)-2-[(tert-butoxycarbonyl)amino]-3-hydroxybutanoate (11.5 g, 49.300 mmol, 1 equiv), TBSCl (11.15 g, 73.950 mmol, 1.5 equiv), Imidazole (5.03 g, 73.950 mmol, 1.5 equiv) and DCM (100 mL) at room temperature. The resulting mixture was stirred for 1 h at room temperature. The resulting mixture was extracted with DCM (2×100 mL). The combined organic layers were washed with brine (200 mL), dried over anhydrous Na₂SO₄. After filtration, the filtrate was concentrated under reduced pressure. The residue was purified by silica gel column chromatography, eluted with PE/EA (10:1) to afford methyl (2S,3S)-2-[(tert-butoxycarbonyl)amino]-3-[(tert-butyldimethylsilyl)oxy]butanoate (13 g, 75.88%) as a colorless oil.

### Step 3. Synthesis of tert-butyl N-[(2R,3S)-3-[(tert-butyldimethylsilyl)oxy]-1-hydroxybutan-2-yl]carbamate

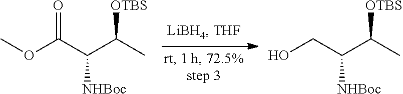 [SMILES: COC(=O)[C@@H](NC(=O)OC(C)(C)C)[C@H](C)O[Si](C)(C)C(C)(C)C ; C[C@H](O[Si](C)(C)C(C)(C)C)[C@@H](CO)NC(=O)OC(C)(C)C]

Into a 500 mL 3-necked round-bottom flask were added methyl (2S,3S)-2-[(tert-butoxycarbonyl)amino]-3-[(tert-butyldimethylsilyl)oxy]butanoate (12 g, 34.530 mmol, 1 equiv), LiBH₄ (2M in THF, 25.90 mL, 51.795 mmol, 1.5 equiv) and THE (120 mL) at 0° C. The resulting mixture was stirred for 1 h at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (5:1) to afford tert-butyl N-[(2R,3S)-3-[(tert-butyldimethylsilyl)oxy]-1-hydroxybutan-2-yl]carbamate (8 g, 72.51%) as a colorless oil.

Reference for tert-butyl (R)-4-((R)-1-((tert-butyldimethylsilyl)oxy)ethyl)-1,2,3-oxathiazolidine-3-carboxylate 2,2-dioxide (AA-26): Gonda, J.; Helland, A-C; Ernst, B.; Bellus, D. Synthesis, 1993, 7, 729-734.

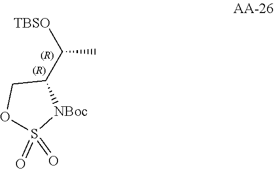

Synthesis of tert-butyl (S)-4-cyclopropyl-1,2,3-oxathiazolidine-3-carboxylate 2,2-dioxide (AA-27) was performed according to the general method starting from (S)-tert-butyl (1-cyclopropyl-2-hydroxyethyl)carbamate (Parker, W. L.; Hanson, R. L.; Goldberg, S. L.; Tully, T. P.; Animesh, G.; Organic Process Research and Development, 2012, 16, 464-469.)

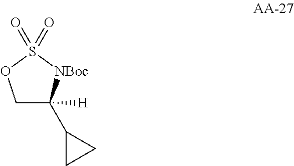 [SMILES: CC(C)(C)OC(=O)N1[C@@H](C2CC2)COS1(=O)=O]

Synthesis of tert-butyl (R)-4-(1-methoxycyclopropyl)-1,2,3-oxathiazolidine-3-carboxylate 2,2-dioxide (AA-28) was performed according to the general method starting from tert-butyl N-[(1R)-2-hydroxy-1-(1-methoxycyclopropyl)ethyl]carbamate.

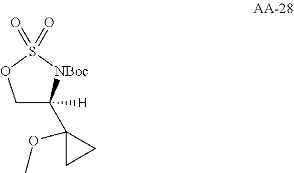 [SMILES: COC1([C@H]2COS(=O)(=O)N2C(=O)OC(C)(C)C)CC1]

### Synthesis of tert-butyl N-[(1R)-2-hydroxy-1-(1-methoxycyclopropyl)ethyl]carbamate

### Step 1. Synthesis of tert-butyl (4R)-4-(1-hydroxycyclopropyl)-2,2-dimethyl-1,3-oxazolidine-3-carboxylate

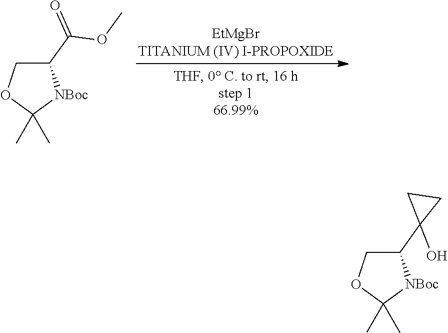

A mixture of 3-tert-butyl 4-methyl (4R)-2,2-dimethyl-1,3-oxazolidine-3,4-dicarboxylate (10.6 g, 40.879 mmol, 1 equiv) and Titanium(IV) isopropoxide (5.81 g, 20.439 mmol, 0.5 equiv) in THE (110 mL) at 0° C. A solution was under nitrogen atmosphere followed by the addition of ethylmagnesium bromide (13.62 g, 102.197 mmol, 2.5 equiv) dropwise at 0° C. The resulting mixture was stirred for 16 h at room temperature under nitrogen atmosphere. The reaction was quenched with sat. NH₄Cl (aq.) at 0° C. The aqueous layer was extracted with EtOAc (3×60 mL). The resulting mixture was concentrated under reduced pressure. The residue was purified by silica gel column chromatography, eluted with PE/EA (4:1) to afford tert-butyl (4R)-4-(1-hydroxycyclopropyl)-2,2-dimethyl-1,3-oxazolidine-3-carboxylate (7.047 g, 66.99%) as a colorless oil.

M/z=258.1 (M+1)⁺

### Step 2. Synthesis of tert-butyl (4R)-4-(1-methoxycyclopropyl)-2,2-dimethyl-1,3-oxazolidine-3-carboxylate

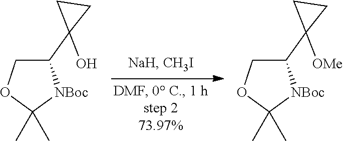

To a mixture of tert-butyl (4R)-4-(1-hydroxycyclopropyl)-2,2-dimethyl-1,3-oxazolidine-3-carboxylate (5.0 g, 19.430 mmol, 1 equiv) in DMF (50 mL) was added NaH (0.93 g, 38.860 mmol, 2 equiv) at 0° C. After the resulting mixture was stirred for 0.5 h at 0° C., CH₃I (3.59 g, 25.259 mmol, 1.3 equiv) was added dropwise at 0° C. The resulting mixture was stirred for 1 h at 0° C. under nitrogen atmosphere and quenched with Water. The aqueous layer was extracted with EtOAc (4×50 mL). The resulting mixture was concentrated under reduced pressure and purified by silica gel column chromatography, eluted with PE/EA (4:1) to afford tert-butyl (4R)-4-(1-methoxycyclopropyl)-2,2-dimethyl-1,3-oxazolidine-3-carboxylate (3.9 g, 73.97%) as a yellow oil.

M/z=272.2 (M+1)⁺

### Step 3. Synthesis of tert-butyl N-[(1R)-2-hydroxy-1-(1-methoxycyclopropyl)ethyl]carbamate

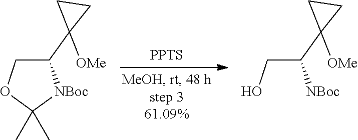

A mixture of tert-butyl (4R)-4-(1-methoxycyclopropyl)-2,2-dimethyl-1,3-oxazolidine-3-carboxylate (2.8 g, 10.319 mmol, 1 equiv) and PPTS (3.94 g, 15.685 mmol, 1.52 equiv) in methanol (50 mL) was stirred for 48 h at room temperature under air atmosphere. The resulting mixture was concentrated under reduced pressure. The residue was purified by silica gel column chromatography, eluted with PE/EA (2:1) to afford tert-butyl N-[(1R)-2-hydroxy-1-(1-methoxycyclopropyl)ethyl]carbamate (1.458 g, 61.09%) as a yellow oil.

Synthesis of tert-butyl (S)-4-(2-(trifluoromethoxy)ethyl)-1,2,3-oxathiazolidine-3-carboxylate 2,2-dioxide (AA-29) was performed according to the general method starting from tert-butyl N-[(2S)-1-hydroxy-4-(trifluoromethoxy)butan-2-yl]carbamate.

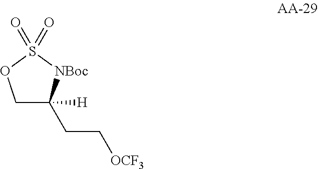 [SMILES: CC(C)(C)OC(=O)N1[C@@H](CCOC(F)(F)F)COS1(=O)=O]

### Synthesis of tert-butyl N-[(2S)-1-hydroxy-4-(trifluoromethoxy)butan-2-yl]carbamate

### Step 1

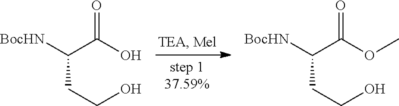 [SMILES: CC(C)(C)OC(=O)N[C@@H](CCO)C(=O)O ; COC(=O)[C@H](CCO)NC(=O)OC(C)(C)C]

To a stirred mixture of (2S)-2-[(tert-butoxycarbonyl)amino]-4-hydroxybutanoic acid (10 g, 45.613 mmol, 1 equiv) and TEA (9.23 g, 91.226 mmol, 2 equiv) in DMF (100 mL) was added Mel (12.95 g, 91.226 mmol, 2 equiv) dropwise at 0° C. under air atmosphere. The resulting mixture was stirred for 3 h at room temperature under air atmosphere. The reaction was quenched with Water at room temperature. The aqueous layer was extracted with EtOAc (4×50 mL). The residue was purified by silica gel column chromatography, eluted with PE/EA (1:1) to afford methyl (2S)-2-[(tert-butoxycarbonyl)amino]-4-hydroxybutanoate (4 g, 37.59%) as a light yellow oil.

### Step 2. Synthesis of methyl (2S)-2-[(tert-butoxycarbonyl)amino]-4-(trifluoromethoxy)butanoate

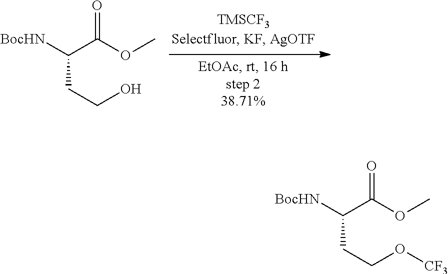 [SMILES: COC(=O)[C@H](CCO)NC(=O)OC(C)(C)C ; COC(=O)[C@H](CCOC(F)(F)F)NC(=O)OC(C)(C)C]

To a stirred mixture of methyl (2S)-2-[(tert-butoxycarbonyl)amino]-4-hydroxybutanoate (8 g, 34.296 mmol, 1 equiv), trifluoromethyltrimethylsilane (14.63 g, 102.888 mmol, 3 equiv), silver (1+) trifluoromethanesulfonate (26.44 g, 102.888 mmol, 3 equiv), KF (7.97 g, 137.184 mmol, 4 equiv), Selectfluor (18.22 g, 51.444 mmol, 1.5 equiv) and 2-fluoropyridine (9.99 g, 102.888 mmol, 3 equiv) in EA (80 mL) at room temperature under an air atmosphere. The resulting mixture was stirred for 16 h at room temperature under a nitrogen atmosphere. The resulting mixture was filtered, the filter cake was washed with EtOAc (3×30 mL). The filtrate was concentrated under reduced pressure and purified by silica gel column chromatography, eluted with PE/EA (5:1) to afford methyl (2S)-2-[(tert-butoxycarbonyl)amino]-4-(trifluoromethoxy)butanoate (4 g, 38.71%) as a light yellow oil.

### Step 3. Synthesis of

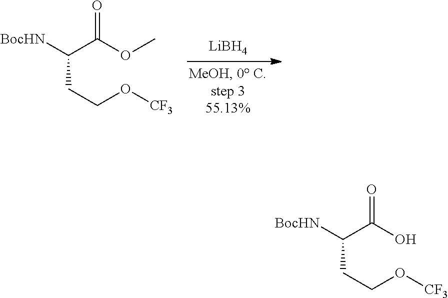 [SMILES: COC(=O)[C@H](CCOC(F)(F)F)NC(=O)OC(C)(C)C ; "LiBH4" ; CC(C)(C)OC(=O)N[C@@H](CCOC(F)(F)F)C(=O)O]

To a stirred mixture of methyl (2S)-2-[(tert-butoxycarbonyl)amino]-4-(trifluoromethoxy)butanoate (4 g, 13.277 mmol, 1 equiv) in THE (40 mL) were added a mixture of LiBH₄ in THF (19.4 mL, 39.831 mmol, 3 equiv, 2 mol/L) dropwise at 0° C. under a nitrogen atmosphere. The resulting mixture was stirred for 3 h at room temperature under a nitrogen atmosphere. The reaction was quenched with water at room temperature and extracted with EtOAc (3×150 mL). The residue was purified by silica gel column chromatography, eluted with PE/EA (4:1) to afford tert-butyl N-[(2S)-1-hydroxy-4-(trifluoromethoxy)butan-2-yl]carbamate (2 g, 55.13%) as a light yellow oil.

Synthesis of tert-butyl (S)-4-(2-methoxyethyl)-1,2,3-oxathiazolidine-3-carboxylate 2,2-dioxide (AA-30) was performed according to the general method starting from commercially available (2S)-2-amino-4-methoxybutan-1-ol.

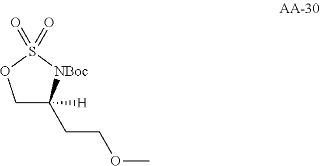 [SMILES: COCC[C@H]1COS(=O)(=O)N1C(=O)OC(C)(C)C]

Synthesis of tert-butyl (S)-4-(cyanomethyl)-1,2,3-oxathiazolidine-3-carboxylate 2,2-dioxide (AA-31) was made from commerically available tert-butyl [(2S)-1-cyano-3-hydroxypropan-2-yl]carbamate according to the general method.

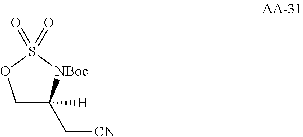 [SMILES: CC(C)(C)OC(=O)N1[C@@H](CC#N)COS1(=O)=O]

Synthesis of tert-butyl (S)-4-((methoxy-d3)methyl)-1,2,3-oxathiazolidine-3-carboxylate 2,2-dioxide (AA-32) was performed according to the general method starting from tert-butyl (4S)-4-[(2H3)methoxymethyl]-2-oxo-1,2lambda4,3-oxathiazolidine-3-carboxylate.

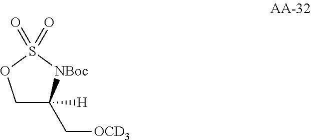

### Synthesis of tert-butyl (4S)-4-[(2H3)methoxymethyl]-2-oxo-1,2lambda4,3-oxathiazolidine-3-carboxylate

### Step 1. Synthesis of methyl (2S)-2-[(tert-butoxycarbonyl)amino]-3-(2H3)methoxypropanoate

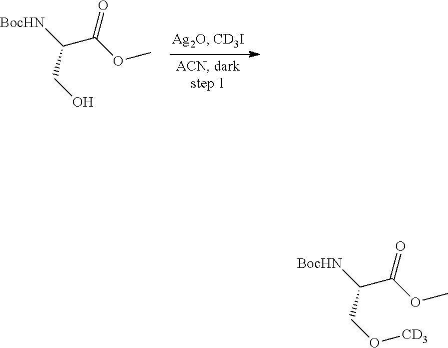 [SMILES: COC(=O)[C@H](CO)NC(=O)OC(C)(C)C ; [2H]C([2H])([2H])OC[C@H](NC(=O)OC(C)(C)C)C(=O)OC]

A mixture of methyl (2S)-2-[(tert-butoxycarbonyl) amino]-3-hydroxypropanoate (5 g, 22.806 mmol, 1 equiv), CD₃I (33.72 g, 232.621 mmol, 10.2 equiv) and Ag₂O (26.95 g, 116.311 mmol, 5.1 equiv) in ACN (100 mL) was stirred for 3 days at room temperature under nitrogen atmosphere in dark. The resulting mixture was filtered and the filter cake was washed with EtOAc (3×100 mL). The filtrate was concentrated under reduced pressure. The residue was purified by silica gel column chromatography, eluted with PE/EA (1:1) to afford methyl (2S)-2-[(tert-butoxycarbonyl)amino]-3-(2H3)methoxypropanoate (5 g, 92.79%) as a colorless oil.

¹H NMR (300 MHz, Chloroform-d) δ 5.39 (d, J=8.8 Hz, 1H) 4.43 (dd, J=8.7, 3.5 Hz, 1H), 3.78 (s, 3H), 3.60 (dd, J=9.4, 3.4 Hz, 1H), 3.31-3.21 (m, 2H), 1.38 (s, 9H).

### Step 2. Synthesis of afford tert-butyl N-[(2R)-1-hydroxy-3-(2H3) methoxypropan-2-yl]carbamate

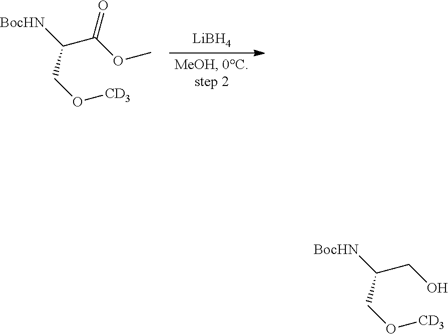 [SMILES: [2H]C([2H])([2H])OC[C@H](NC(=O)OC(C)(C)C)C(=O)OC ; "LiBH4" ; [2H]C([2H])([2H])OC[C@@H](CO)NC(=O)OC(C)(C)C]

To a stirred solution of methyl (2S)-2-[(tert-butoxycarbonyl)amino]-3-(2H3)methoxypropanoate (5.4 g, 22.854 mmol, 1 equiv) in MeOH (30 mL) and THE (30 mL) was added 2M LiBH₄ (1.00 g, 45.708 mmol, 2 equiv) dropwise at 5 min at 0° C. under nitrogen atmosphere. The resulting mixture was stirred overnight at room temperature. The resulting mixture was filtered and the filter cake was washed with THE (2×50 mL). The filtrate was concentrated under reduced pressure. The residue was purified by silica gel column chromatography, eluted with PE/EA (1:1) to afford tert-butyl N-[(2R)-1-hydroxy-3-(2H3) methoxypropan-2-yl]carbamate (4.3 g, 90.34%) as a light yellow oil.

¹H NMR (300 MHz, DMSO-d6) δ 6.48 (d, J=8.4 Hz, 1H), 4.60 (t, J=5.7 Hz, 1H), 3.54 (q, J=7.5, 6.6 Hz, 1H), 3.34 (d, J=1.6 Hz, 1H), 3.31-3.21 (m, 2H), 1.38 (s, 9H).

Reference for tert-butyl (R)-4-((R)-tetrahydrofuran-2-yl)-1,2,3-oxathiazolidine-3-carboxylate 2,2-dioxide (AA-33): (WO2021/113492)

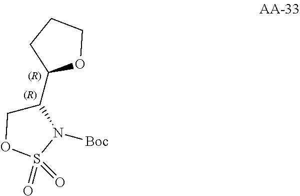

Synthesis of tert-butyl (S)-4-((2,2-difluoroethoxy)methyl)-1,2,3-oxathiazolidine-3-carboxylate 2,2-dioxide (AA-34) was performed according to the general method starting from tert-butyl (R)-(1-(2,2-difluoroethoxy)-3-hydroxypropan-2-yl)carbamate.

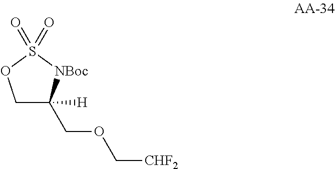 [SMILES: CC(C)(C)OC(=O)N1[C@@H](COCC(F)F)COS1(=O)=O]

### Synthesis of tert-butyl N-[(2R)-1-(2,2-difluoroethoxy)-3-hydroxypropan-2-yl]carbamate

### Step 1. Synthesis of methyl (2S)-2-[(tert-butoxycarbonyl)amino]-3-(2,2-difluoroethoxy)propanoate

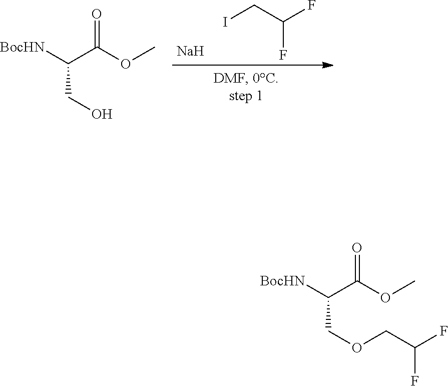

To a solution of methyl (2S)-2-[(tert-butoxycarbonyl)amino]-3-hydroxypropanoate (7 g, 31.929 mmol, 1 equiv) in THE (70 mL) was added NaH (1.15 g, 47.893 mmol, 1.5 equiv) in portion, the mixture was stirred for 30 min at 0° C. under air atmosphere. To the above mixture was added 2,2-difluoroethyl trifluoromethanesulfonate (8887.23 mg, 41.508 mmol, 1.3 equiv) dropwise at 0° C. The resulting mixture was stirred for additional 2 h at room temperature. The reaction was quenched with water (100 mL) at 0° C. and extracted with EtOAc (3×150 mL). The combined organic layers were washed with water (2×60 mL), dried over anhydrous Na₂SO₄. After filtration, the filtrate was concentrated under reduced pressure. The residue was purified by silica gel column chromatography, eluted with PE/EA (10:1) to afford methyl (2S)-2-[(tert-butoxycarbonyl)amino]-3-(2,2-difluoroethoxy)propanoate (1.5 g, 16.58%) as a colourless oil.

### Step 2. Synthesis of tert-butyl N-[(2R)-1-(2,2-difluoroethoxy)-3-hydroxypropan-2-yl]carbamate

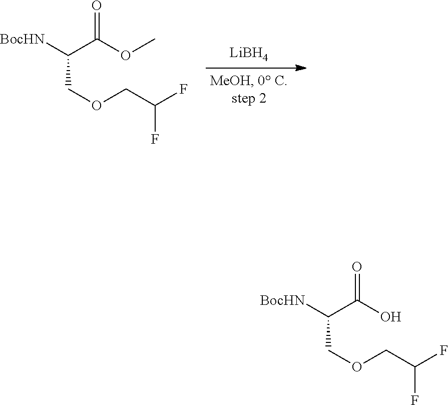 [SMILES: COC(=O)[C@H](COCC(F)F)NC(=O)OC(C)(C)C ; "LiBH4" ; CC(C)(C)OC(=O)N[C@@H](COCC(F)F)C(=O)O]

To a stirred solution of methyl (2S)-2-[(tert-butoxycarbonyl)amino]-3-(2,2-difluoroethoxy)propanoate (1.5 g, 5.295 mmol, 1 equiv) in MeOH (15 mL) were added LiBH₄ (230.66 mg, 10.590 mmol, 2.00 equiv) dropwise at 0° C. under air atmosphere. The resulting mixture was stirred overnight at room temperature. The resulting mixture was filtered, the filter cake was washed with MeOH (2×50 mL). The filtrate was concentrated under reduced pressure. The resulting mixture was diluted with EtOAc (200 mL), washed with water (2×50 mL) and dried over anhydrous Na₂SO₄. After filtration, the filtrate was concentrated under reduced pressure. The residue was purified by silica gel column chromatography, eluted with PE/EA (1:1) to afford tert-butyl N-[(2R)-1-(2,2-difluoroethoxy)-3-hydroxypropan-2-yl]carbamate (800 mg, 59.19%) as a colourless oil.

Synthesis of tert-butyl (4S)-4-(2,2-difluorocyclopropyl)-1,2,3-oxathiazolidine-3-carboxylate 2,2-dioxide (AA-35) was performed according to the general method starting from tert-butyl ((1S)-1-(2,2-difluorocyclopropyl)-2-hydroxyethyl)carbamate

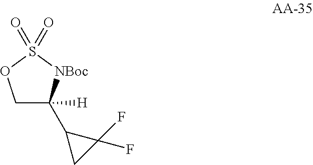 [SMILES: CC(C)(C)OC(=O)N1[C@@H](C2CC2(F)F)COS1(=O)=O]

### Synthesis of tert-butyl N-[1-(2,2-difluorocyclopropyl)-3-hydroxypropan-2-yl]carbamate

### Step 1. Synthesis of (2,2-difluorocyclopropyl)methyl 4-methylbenzenesulfonate

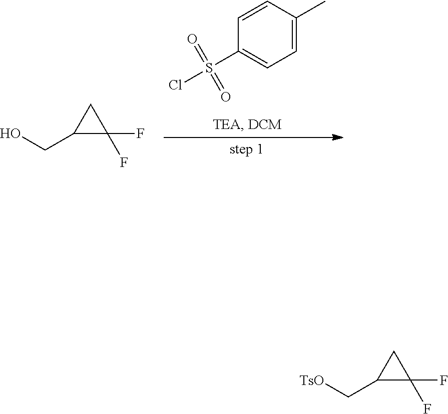 [SMILES: Cc1ccc(S(=O)(=O)Cl)cc1 ; OCC1CC1(F)F ; Cc1ccc(S(=O)(=O)OCC2CC2(F)F)cc1]

A solution of (2,2-difluorocyclopropyl)methanol (5 g, 46.259 mmol, 1 equiv) and TEA (9.36 g, 92.518 mmol, 2 equiv) in DCM (50 mL) was stirred for 15 min at −10° C. under nitrogen atmosphere. Then a solution of 4-methylbenzene-1-sulfonyl chloride (17.64 g, 92.518 mmol, 2 equiv) in DCM (50 mL) was added dropwise at −10° C. The resulting mixture was stirred for additional 1.5 h at 0° C. The resulting mixture was concentrated under reduced pressure. The residue was purified by silica gel column chromatography, eluted with PE/EA (4:1) to afford (2,2-difluorocyclopropyl)methyl 4-methylbenzenesulfonate (10.5 g, 86.55%) as a colourless oil.

¹H NMR (300 MHz, Chloroform-d) δ 7.86-7.79 (m, 2H), 7.42-7.34 (m, 2H), 4.11 (dt, J=7.8, 1.6 Hz, 2H), 2.48 (s, 3H), 2.02-1.89 (m, 1H), 1.60-1.12 (m, 2H).

### Step 2. Synthesis of tert-butyl 3-(2,2-difluorocyclopropyl)-2-[(diphenylmethylidene)amino]propanoate

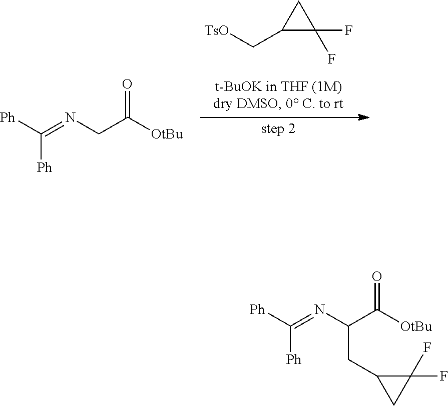 [SMILES: Cc1ccc(S(=O)(=O)OCC2CC2(F)F)cc1 ; CC(C)(C)OC(=O)CN=C(c1ccccc1)c1ccccc1 ; CC(C)(C)OC(=O)C(CC1CC1(F)F)N=C(c1ccccc1)c1ccccc1]

To a stirred solution of tert-butyl 2-[(diphenylmethylidene)amino]acetate (10 g, 33.854 mmol, 1 equiv) in DMSO (10 mL) was added t-BuOK (4.06 g, 36.224 mmol, 1.07 equiv) in DMSO (50 mL) dropwise at 15° C. under nitrogen atmosphere. To the above mixture was added a solution of (2,2-difluorocyclopropyl)methyl 4-methylbenzenesulfonate (10.12 g, 38.594 mmol, 1.14 equiv) in DMSO (100 mL) dropwise for 10 min at 15° C. The reaction was quenched with Water at room temperature. The resulting mixture was extracted with EtOAc (3×100 mL). The combined organic layers were washed with brine (1×70 mL), dried over anhydrous Na₂SO₄. After filtration, the filtrate was concentrated under reduced pressure. The residue was purified by silica gel column chromatography, eluted with PE/EA (4:1) to afford tert-butyl 3-(2,2-difluorocyclopropyl)-2-[(diphenylmethylidene)amino]propanoate (6 g, 45.98%) as a light yellow oil.

M/z [M+H]⁺=386

### Step 3. Synthesis of tert-butyl 2-amino-3-(2,2-difluorocyclopropyl) propanoate

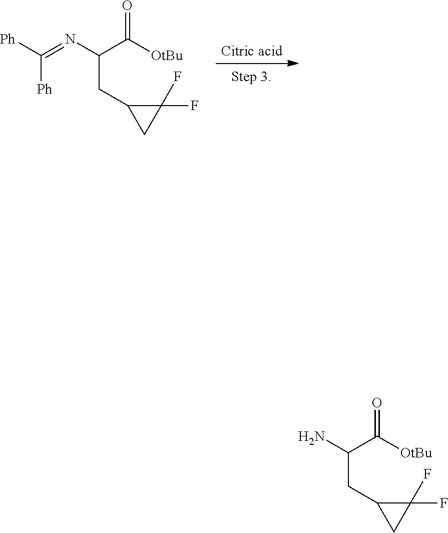 [SMILES: CC(C)(C)OC(=O)C(CC1CC1(F)F)N=C(c1ccccc1)c1ccccc1 ; CC(C)(C)OC(=O)C(N)CC1CC1(F)F]

A mixture of tert-butyl 3-(2,2-difluorocyclopropyl)-2-[(diphenylmethylidene)amino]propanoate (6 g, 15.566 mmol, 1 equiv) and 15% wt citric acid (400 mL, 6226.400 mmol) in THE (60 mL) was stirred for overnight at room temperature under nitrogen atmosphere. The resulting mixture was concentrated under reduced pressure. The resulting mixture was diluted with water (100 mL). The mixture was acidified to pH 2 with 1M HCl (aq.). The resulting mixture was extracted with EtOAc (2×100 mL). The water phase was basified to pH 8 with saturated k₂CO₃ (aq.). The resulting mixture was extracted with EtOAc (3×200 mL). The combined organic layers were washed with brine (2×40 mL), dried over anhydrous Na₂SO₄. After filtration, the filtrate was concentrated under reduced pressure. This resulted in tert-butyl 2-amino-3-(2,2-difluorocyclopropyl) propanoate (3.45 g, 99.18%) as a light yellow oil.

### Step 4. Synthesis of afford tert-butyl 2-[(tert-butoxycarbonyl)amino]-3-(2,2-difluorocyclopropyl)propanoate

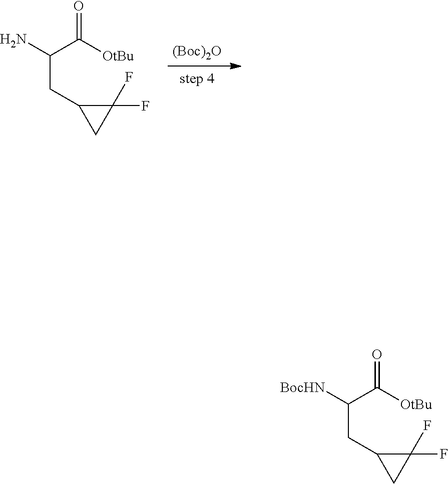 [SMILES: "(Boc)2O" ; CC(C)(C)OC(=O)C(N)CC1CC1(F)F ; CC(C)(C)OC(=O)NC(CC1CC1(F)F)C(=O)OC(C)(C)C]

A mixture of tert-butyl 2-amino-3-(2,2-difluorocyclopropyl)propanoate (3.45 g, 15.593 mmol, 1 equiv) and TEA (2.37 g, 23.389 mmol, 1.5 equiv) in DCM (50 mL) was stirred for overnight at room temperature under air atmosphere. The resulting mixture was washed with 2×30 mL of water. The resulting mixture was concentrated under reduced pressure. The residue was purified by silica gel column chromatography, eluted with PE/EA (4:1) to afford tert-butyl 2-[(tert-butoxycarbonyl)amino]-3-(2,2-difluorocyclopropyl)propanoate (4 g, 79.82%) as a light yellow oil.

1H NMR (300 MHz, DMSO-d6) δ 7.25 (d, J=7.8 Hz, 1H), 3.92-3.76 (m, 1H), 1.87-1.62 (m, 2H), 1.44 (d, J=23.0 Hz, 18H).

### Step 5. Synthesis of tert-butyl N-[1-(2,2-difluorocyclopropyl)-3-hydroxypropan-2-yl]carbamate

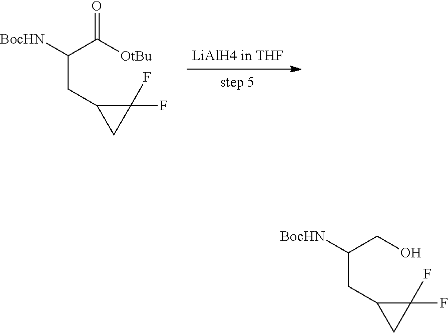 [SMILES: CC(C)(C)OC(=O)NC(CC1CC1(F)F)C(=O)OC(C)(C)C ; CC(C)(C)OC(=O)NC(CO)CC1CC1(F)F]

To a stirred solution of tert-butyl 2-[(tert-butoxycarbonyl)amino]-3-(2,2-difluorocyclopropyl)propanoate (4 g, 12.447 mmol, 1 equiv) in THE (100 mL) was added LiAlH4 (0.94 g, 24.894 mmol, 2 equiv) in THF (12.4 mL) dropwise at 0° C. under nitrogen atmosphere. The resulting mixture was stirred for overnight at room temperature. The reaction was quenched by the addition of Water (1 mL) at 0° C. The resulting mixture was filtered and the filter cake was washed with THF (2×50 mL). The filtrate was concentrated under reduced pressure. The residue was purified by silica gel column chromatography, eluted with PE/EA (1:1) to afford tert-butyl N-[1-(2,2-difluorocyclopropyl)-3-hydroxypropan-2-yl]carbamate (2.7 g, 86.33%) as a colourless oil. 1H NMR (300 MHz, DMSO-d6) δ 6.57 (dd, J=14.9, 8.6 Hz, 1H), 4.64 (s, 1H), 3.61-3.37 (m, 1H), 3.27 (d, J=7.5 Hz, 1H), 1.38 (s, 13H), 1.24-0.83 (m, 2H).

Reference for (S)-4-methyl-1,3,2-dioxathiolane 2,2-dioxide (AA-36): Bates, R. W.; Lu, Y. Organic Letters, 2010, 12, 3938-3941.

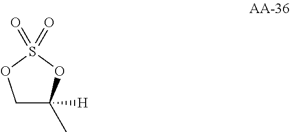

Synthesis of tert-butyl (S)-4-methyl-1,2,3-oxathiazolidine-3-carboxylate-4-d 2,2-dioxide (AA-37) was performed according to the general method starting from tert-butyl N-[1-hydroxy(3,3,3-2H3)propan-2-yl]carbamate

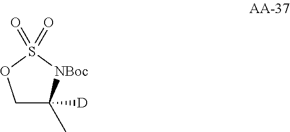 [SMILES: [2H][C@]1(C)COS(=O)(=O)N1C(=O)OC(C)(C)C]

### Synthesis of tert-butyl N-[1-hydroxy(3,3,3-2H3)propan-2-yl]carbamate

### Step 1. Synthesis of L-alanine-2-d

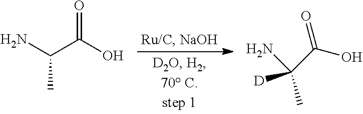

To a mixture of L-alanine (10 g, 112.241 mmol, 1 equiv) in deuterium oxide (90 mL) was added NaOH (13.47 g, 336.723 mmol, 3 equiv) and Ru/C (1.13 g, 11.224 mmol, 0.1 equiv) in a pressure tank. The mixture was hydrogenated at 70° C. under 1 atm of hydrogen pressure for overnight. The mixture was allowed to cool down to room temperature. The resulting mixture was filtered and the crude resulting mixture was used in the next step directly without further purification.

1H NMR (400 MHz, Deuterium Oxide) δ 1.15-1.04 (m, 3H).

### Step 2. Synthesis of (2S)-2-[(tert-butoxycarbonyl)amino](2-2H)propanoic acid

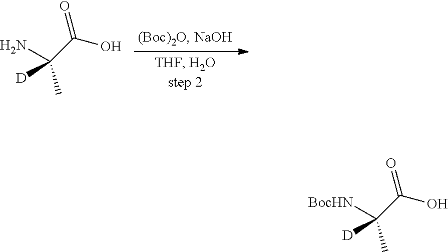

A mixture of (2S)-2-amino(2-2H)propanoic acid (10 g, 110.988 mmol, 1 equiv), NaOH (13.32 g, 332.964 mmol, 3 equiv) and (Boc)₂O (36.33 g, 166.482 mmol, 1.5 equiv) in THF (100 mL) and H₂O (100 mL) was stirred for overnight at room temperature under air atmosphere. The aqueous layer was extracted with PE (2×50 mL). The residue was acidified to pH 4 with 2N HCl. The resulting mixture was extracted with EtOAc (3×100 mL). The combined organic layers were washed with brine (1×50 mL), dried over anhydrous Na₂SO₄. After filtration, the filtrate was concentrated under reduced pressure to afford (2S)-2-[(tert-butoxycarbonyl)amino](2-2H)propanoic acid as an off-white solid. The crude resulting mixture was used in the next step directly without further purification.

### Synthesis of tert-butyl N-[(2S)-1-hydroxy(2-2H)propan-2-yl]carbamate

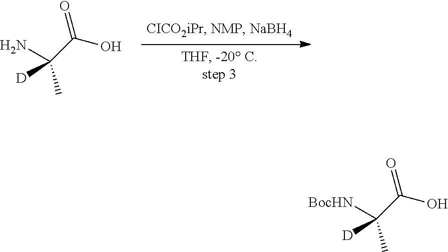

To a stirred mixture of (2S)-2-[(tert-butoxycarbonyl)amino](2-2H)propanoic acid (5 g, 26.286 mmol, 1 equiv) and 4-methylmorpholine (3988.09 mg, 39.429 mmol, 1.5 equiv) in THF (100 mL) was added isopropyl chloroformate (4831.98 mg, 39.429 mmol, 1.5 equiv) dropwise at 0° C. under nitrogen atmosphere. The resulting mixture was stirred for 1 h at 0° C. under nitrogen atmosphere. To the above mixture was added LiBH₄ (17.09 mL, 34.172 mmol, 1.3 equiv) dropwise over 10 min at 0° C. The resulting mixture was stirred for additional 2 h at room temperature. The reaction was quenched with Water/Ice at room temperature. The resulting mixture was extracted with EtOAc (3×150 mL). The combined organic layers were washed with brine (1×50 mL), dried over anhydrous Na₂SO₄. After filtration, the filtrate was concentrated under reduced pressure to afford tert-butyl N-[(2S)-1-hydroxy(2-2H)propan-2-yl]carbamate (1.5 g, 32.38%) as a yellow oil.

Synthesis of tert-butyl 4-(methyl-d3)-1,2,3-oxathiazolidine-3-carboxylate 2,2-dioxide (AA-38) was performed according to the general method starting from tert-butyl N-[1-hydroxy(3,3,3-2H3)propan-2-yl]carbamate.

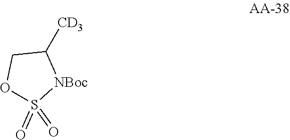 [SMILES: [2H]C([2H])([2H])C1COS(=O)(=O)N1C(=O)OC(C)(C)C]

### Synthesis of tert-butyl N-[1-hydroxy(3,3,3-2H3)propan-2-yl]carbamate

### Step 1. Synthesis of tert-butyl 2-[(diphenylmethylidene)amino](3,3,3-2H3)propanoate

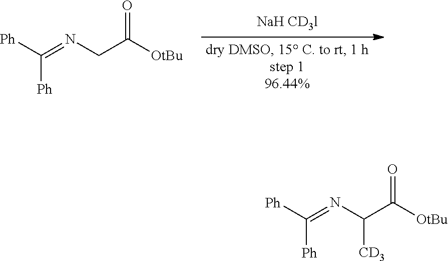 [SMILES: CC(C)(C)OC(=O)CN=C(c1ccccc1)c1ccccc1 ; [2H]C([2H])([2H])C(N=C(c1ccccc1)c1ccccc1)C(=O)OC(C)(C)C]

To a mixture of tert-butyl 2-[(diphenylmethylidene)amino]acetate (15 g, 50.782 mmol, 1 equiv) in DMSO (150 mL) was added NaH (1.22 g, 50.782 mmol, 1 equiv) at 15° C. The resulting mixture was stirred for 0.5 h at 15° C. and CD₃I (7.36 g, 50.782 mmol, 1 equiv) was added dropwise. The resulting mixture was stirred for 1 h at room temperature under nitrogen atmosphere and quenched by the addition of Water (50 mL) at 15° C. The aqueous layer was extracted with EtOAc (4×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (4:1) to afford tert-butyl 2-[(diphenylmethylidene)amino](3,3,3-2H3)propanoate (15.3 g, 96.44%) as a yellow oil.

M/z=313.1 (M+1)⁺

### Step 2. Synthesis of tert-butyl 2-amino(3,3,3-2H3)propanoate

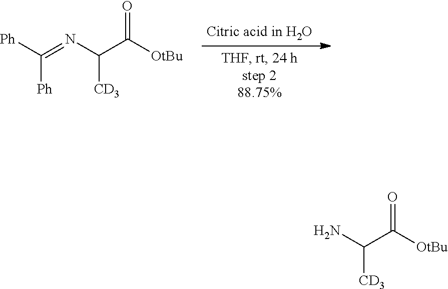 [SMILES: [2H]C([2H])([2H])C(N=C(c1ccccc1)c1ccccc1)C(=O)OC(C)(C)C ; [2H]C([2H])([2H])C(N)C(=O)OC(C)(C)C]

A mixture of tert-butyl 2-[(diphenylmethylidene)amino](3,3,3-2H3)propanoate (15.2 g, 48.651 mmol, 1 equiv) and 2-hydroxypropane-1,2,3-tricarboxylic acid (16.08 g, 83.680 mmol, 1.72 equiv) in H₂O (91.2 mL) and THF (152 mL) was stirred for 24 h at room temperature under air atmosphere. The mixture was basified to pH 9 with 2N NaOH. The aqueous layer was extracted with EtOAc (3×100 mL) and the combined organic layers were washed with brine (2×50 mL), dried over anhydrous Na₂SO₄. After filtration, the filtrate was concentrated under reduced pressure to afford tert-butyl 2-amino(3,3,3-2H3)propanoate (6.4 g, 88.75%) as a yellow oil.

### Step 3. Synthesis of tert-butyl 2-[(tert-butoxycarbonyl)amino](3,3,3-2H3)propanoate

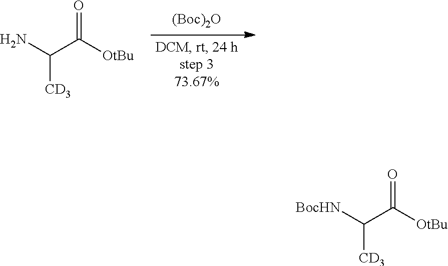 [SMILES: "(Boc)2O" ; [2H]C([2H])([2H])C(N)C(=O)OC(C)(C)C ; [2H]C([2H])([2H])C(NC(=O)OC(C)(C)C)C(=O)OC(C)(C)C]

To a solution of tert-butyl 2-amino(3,3,3-2H3)propanoate (6.4 g, 43.179 mmol, 1 equiv) in DCM (64 mL) was added TEA (6.55 g, 64.769 mmol, 1.5 equiv) and (Boc)₂O (14.14 g, 64.769 mmol, 1.5 equiv) at 20° C. The resulting mixture was stirred for 24 h at room temperature under air atmosphere. The resulting mixture was diluted with 100 mL DCM and washed with 50 mL of water. The combined organic layers were washed with brine (2×20 mL), dried over anhydrous Na₂SO₄. After filtration, the filtrate was concentrated under reduced pressure. The residue was purified by silica gel column chromatography, eluted with PE/EA (4:1) to afford tert-butyl 2-[(tert-butoxycarbonyl)amino](3,3,3-2H3)propanoate (7.9 g, 73.67%) as a yellow oil.

M/z=249.1 (M+1)

### Step 4. Synthesis of tert-butyl N-[1-hydroxy(3,3,3-2H3)propan-2-yl]carbamate

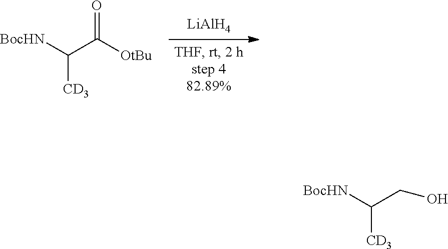 [SMILES: [2H]C([2H])([2H])C(NC(=O)OC(C)(C)C)C(=O)OC(C)(C)C ; "LiAlH4" ; [2H]C([2H])([2H])C(CO)NC(=O)OC(C)(C)C]

A solution of tert-butyl 2-[(tert-butoxycarbonyl)amino](3,3,3-2H3)propanoate (7.9 g, 31.812 mmol, 1 equiv) in THF (50 mL) was added LiAlH₄ (2.41 g, 63.624 mmol, 2 equiv) in portion at 0° C. The resulting mixture was stirred for 2 h at room temperature under nitrogen atmosphere. The reaction was quenched by the addition of Water (2.5 mL) and NaOH (2.5 mL, 1 N) dropwise at 0° C. The resulting mixture was filtered, the filter cake was washed with THF (2×70 mL). The filtrate was concentrated under reduced pressure and the residue was purified by silica gel column chromatography, eluted with PE/EA (1:1) to afford tert-butyl N-[1-hydroxy(3,3,3-2H3)propan-2-yl]carbamate (4.7 g, 82.89%) as a yellow oil.

M/z=179.1 (M+1)+

Synthesis of tert-butyl (S)-4-(2-(methoxy-d3)ethyl)-1,2,3-oxathiazolidine-3-carboxylate 2,2-dioxide was performed according to the general method starting from tert-butyl (4S)-4-[2-(2H3)methoxyethyl]-2,2-dioxo-1,2lambda6,3-oxathiazolidine-3-carboxylate.

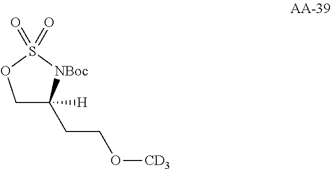 [SMILES: [2H]C([2H])([2H])OCC[C@H]1COS(=O)(=O)N1C(=O)OC(C)(C)C]

### Synthesis of tert-butyl (4S)-4-[2-(2H3)methoxyethyl]-2,2-dioxo-1,2lambda6,3-oxathiazolidine-3-carboxylate

### Step 1. Synthesis of (4S)-4-[2-(2H3)methoxyethyl]-2,2-dimethyl-1,3-oxazolidine-3-carboxylate

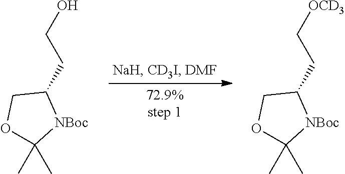

To a solution of tert-butyl (4S)-4-(2-hydroxyethyl)-2,2-dimethyl-1,3-oxazolidine-3-carboxylate (10 g, 40.763 mmol, 1 equiv) in DMF (100 mL) was added NaH (2.45 g, 61.144 mmol, 1.5 equiv, 60%) at 0 degrees C. The mixture was stirred for 30 min. CD₃I (3.04 mL, 48.916 mmol, 1.2 equiv) was added and the mixture was allowed to warm to RT and stirred for 2 h. The reaction was quenched with sat. NH₄Cl (aq.)/Ice (50 mL) at room temperature. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (9:1) to afford tert-butyl (4S)-4-[2-(2H3)methoxyethyl]-2,2-dimethyl-1,3-oxazolidine-3-carboxylate (7.8 g, 72.93%) as a yellow oil.

### Step 2. Synthesis of tert-butyl N-[(2S)-1-hydroxy-4-(2H3)methoxybutan-2-yl]carbamate

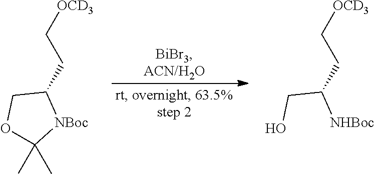

Into a 250-mL 3-necked round-bottom flask were added tert-butyl (4S)-4-[2-(2H3)methoxyethyl]-2,2-dimethyl-1,3-oxazolidine-3-carboxylate (7.8 g, 29.730 mmol, 1 equiv) and BISMUTH(III) BROMIDE (2667.89 mg, 5.946 mmol, 0.2 equiv) in ACN (156 mL) and H₂O (4 mL). The resulting mixture was stirred for 2 h. The reaction mixture was then quenched by addition of saturated aqueous NaHCO₃ (200 mL) and filtered through celite. The filtrate was transferred to a separatory funnel, the layers were separated, and the organic layer was further extracted with EtOAc (2×200 mL). The combined organic extracts were washed with brine (200 mL), dried over Na₂SO₄, filtered and concentrated. The residue was purified by silica gel column chromatography, eluted with PE/EA (1:1) to afford tert-butyl N-[(2S)-1-hydroxy-4-(2H3)methoxybutan-2-yl]carbamate (4.2 g, 63.55%) as a colorless oil.

Synthesis of tert-butyl (S)-4-((methoxy-d3)methyl)-1,2,3-oxathiazolidine-3-carboxylate-4-d 2,2-dioxide (AA-40) was performed according to the general method starting from tert-butyl N-[(2R)-1-hydroxy-3-(2H3)methoxy(2-2H)propan-2-yl]carbamate.

### Synthesis of tert-butyl N-[(2R)-1-hydroxy-3-(2H3)methoxy(2-2H)propan-2-yl]carbamate

### Step 1. Synthesis of methyl (2S)-2-[(tert-butoxycarbonyl)amino]-3-(2H3)methoxypropanoate

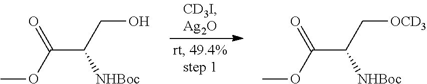

To a stirred solution of methyl (2S)-2-[(tert-butoxycarbonyl)amino]-3-hydroxypropanoate (15 g, 68.419 mmol, 1 equiv) and Ag₂O (79.28 g, 342.095 mmol, 5 equiv) in MeCN (150 mL) were added CD₃I (42.57 mL, 684.190 mmol, 10 equiv) at room temperature under nitrogen atmosphere. The resulting mixture was stirred for 3 days. The reaction was quenched with water. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:1) to afford methyl (2S)-2-[(tert-butoxycarbonyl)amino]-3-(2H3)methoxypropanoate (8 g, 49.49%) as a white solid.

### Step 2. Synthesis of methyl O-(methyl-d3)-L-serinate Hydrochloride Salt

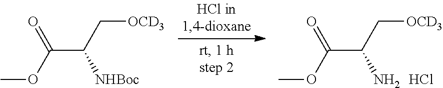

Into a 100-mL round-bottom flask were added methyl (2S)-2-[(tert-butoxycarbonyl)amino]-3-(2H3)methoxypropanoate (3.3 g, 13.966 mmol, 1 equiv) and HCl(gas) in 1,4-dioxane (10 mL) at room temperature for 1 h. The crude product was used in the next step directly without further purification.

### Step 3. Synthesis of (2S)-2-amino-3-(2H3)methoxy(2-2H)propanoic acid

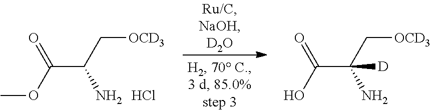

A solution of methyl (2S)-2-amino-3-(2H3)methoxypropanoate (6.5 g, 47.736 mmol, 1 equiv), NaOH (7.64 g, 190.944 mmol, 4 equiv) and Ru/C (30%, 1.95 g) in D₂O (65 mL) was stirred for 3 days at 70° C. under hydrogen atmosphere. The resulting mixture was filtered, the filter cake was washed with D₂O. The filtrate was concentrated under reduced pressure. The residue was purified by silica gel column chromatography, eluted with PE/EA (5:1) to afford (2S)-2-amino-3-(2H3)methoxy(2-2H)propanoic acid (5 g, 85.06%) as a colorless oil.

### Step 4. Synthesis of (2S)-2-[(tert-butoxycarbonyl)amino]-3-(2H3)methoxy(2-2H)propanoic acid

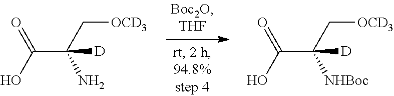

Into a 250-mL round-bottom flask were added (2S)-2-amino-3-(2H3)methoxy(2-2H)propanoic acid (5 g, 40.603 mmol, 1 equiv) and di-tert-butyl dicarbonate (13.29 g, 60.904 mmol, 1.5 equiv) in THE (50 mL) at room temperature stirred for 2 h. The resulting mixture was concentrated under reduced pressure. The residue was purified by silica gel column chromatography, eluted with PE/EA (5:1) to afford (2S)-2-[(tert-butoxycarbonyl)amino]-3-(2H3)methoxy(2-2H)propanoic acid (8.6 g, 94.87%) as a colorless oil.

### Step 5. Synthesis of tert-butyl N-[(2R)-1-hydroxy-3-(2H3)methoxy(2-2H)propan-2-yl]carbamate

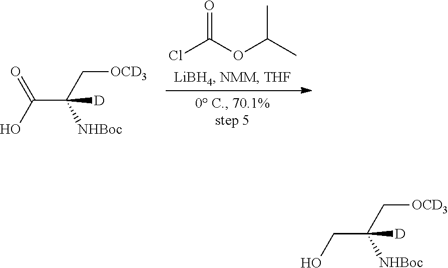

To a stirred solution of (2S)-2-[(tert-butoxycarbonyl)amino]-3-(2H3)methoxy(2-2H)propanoic acid (7.3 g, 32.697 mmol, 1 equiv) and NMM (7.19 mL, 65.394 mmol, 2 equiv) in THE (75 mL) was added isopropyl chloroformate (6.74 mL, 49.046 mmol, 1.5 equiv) dropwise at 0° C. under nitrogen atmosphere. The resulting mixture was stirred for 1 h. To the above mixture was added LiBH₄ (2M in THF, 19.62 mL, 39.236 mmol, 1.2 equiv) dropwise at 0° C. The resulting mixture was stirred for additional 30 min at 0° C. The reaction was quenched with water/Ice. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford tert-butyl N-[(2R)-1-hydroxy-3-(2H3)methoxy(2-2H)propan-2-yl]carbamate (4.8 g, 70.15%) as a colorless oil.

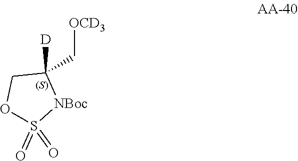

### tert-butyl (S)-4-(methoxymethyl)-1,2,3-oxathiazolidine-3-carboxylate-4-d 2,2-dioxide (AA-41)

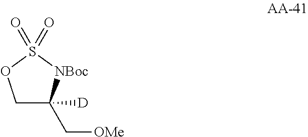 [SMILES: [2H][C@]1(COC)COS(=O)(=O)N1C(=O)OC(C)(C)C]

Synthesis of tert-butyl (S)-4-methyl-1,2,3-oxathiazolidine-3-carboxylate-5,5-d2 2,2-dioxide (AA-42) was performed according to the general method starting from tert-butyl (S)-(1-hydroxyprop-2-yl-1,1-d2)carbamate (Guaragna, A.; Pedatella, S.; Pinto, V. Synthesis, 2006, 23, 4013-4016.

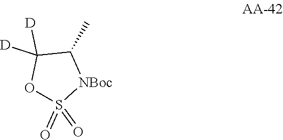 [SMILES: [2H]C1([2H])OS(=O)(=O)N(C(=O)OC(C)(C)C)[C@H]1C]

Synthesis of tert-butyl (S)-4-(methoxymethyl-d2)-1,2,3-oxathiazolidine-3-carboxylate 2,2-dioxide (AA-43) was performed according to the general method starting from tert-butyl N-[(2R)-1-hydroxy-3-(2H3)methoxy(3,3-2H2)propan-2-yl]carbamate.

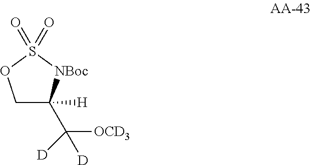

### Synthesis of tert-butyl N-[(2R)-1-hydroxy-3-(2H3)methoxy(3,3-2H2)propan-2-yl]carbamate

### Step 1. Synthesis of tert-butyl (4R)-4-[(2H3)methoxy(2H2)methyl]-2,2-dimethyl-1,3-oxazolidine-3-carboxylate

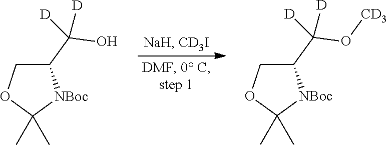

To a stirred mixture of tert-butyl (4R)-4-[hydroxy(2H2)methyl]-2,2-dimethyl-1,3-oxazolidine-3-carboxylate (5 g, 21.431 mmol, 1 equiv) and CD₃I (4.04 g, 27.860 mmol, 1.3 equiv) in DMF (100 mL) was added NaH (0.77 g, 32.147 mmol, 1.5 equiv) dropwise at 0° C. under nitrogen atmosphere. The resulting mixture was stirred for 2 h at room temperature under nitrogen atmosphere. The reaction was quenched with water at 0° C. The mixture was acidified to pH 6 with HCl (aq.). The resulting mixture was extracted with EtOAc (600 mL). The combined organic layers were washed with brine (200 mL), dried over anhydrous Na2SO4. After filtration, the filtrate was concentrated under reduced pressure. The residue was purified by silica gel column chromatography, eluted with PE/EA (10:1) to afford tert-butyl (4R)-4-[(2H3)methoxy(2H2)methyl]-2,2-dimethyl-1,3-oxazolidine-3-carboxylate (4.8 g, 89.46%) as a colourless oil.

### Step 2. Synthesis of tert-butyl N-[(2R)-1-hydroxy-3-(2H3)methoxy(3,3-2H2)propan-2-yl]carbamate

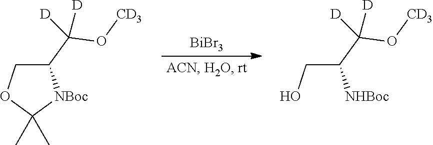

A mixture of tert-butyl (4R)-4-[(2H3)methoxy(2H2)methyl]-2,2-dimethyl-1,3-oxazolidine-3-carboxylate (4 g, 15.978 mmol, 1 equiv) and bismuth tribromide (1.43 g, 3.196 mmol, 0.2 equiv) in MeCN (80 mL) and H₂O (0.8 mL) was stirred for 1 h at room temperature under nitrogen atmosphere. The resulting mixture was extracted with EtOAc (300 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (8:1) to afford tert-butyl N-[(2R)-1-hydroxy-3-(2H3)methoxy(3,3-2H2)propan-2-yl]carbamate (2.5 g, 74.41%) as a light yellow oil.

Synthesis of tert-butyl (S)-4-(methoxymethyl-d2)-1,2,3-oxathiazolidine-3-carboxylate 2,2-dioxide (AA-44) was performed analogously to tert-butyl (S)-4-(methoxymethyl-d2)-1,2,3-oxathiazolidine-3-carboxylate 2,2-dioxide (AA-43).

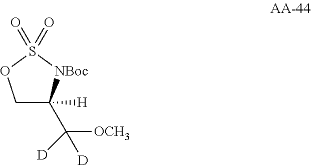 [SMILES: [2H]C([2H])(OC)[C@H]1COS(=O)(=O)N1C(=O)OC(C)(C)C]

Synthesis of tert-butyl (R)-4-((difluoromethoxy)methyl-d2)-2,2-dimethyloxazolidine-3-carboxylate (AA-45) was performed according to the general method starting from tert-butyl N-[(2R)-1-(difluoromethoxy)-3-hydroxy(1,1-2H2)propan-2-yl]carbamate.

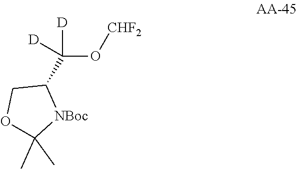 [SMILES: [2H]C([2H])(OC(F)F)[C@H]1COC(C)(C)N1C(=O)OC(C)(C)C]

### Synthesis of tert-butyl N-[(2R)-1-(difluoromethoxy)-3-hydroxy(1,1-2H2)propan-2-yl]carbamate

### Step 1. Synthesis of tert-butyl (4R)-4-[(difluoromethoxy)(2H2)methyl]-2,2-dimethyl-1,3-oxazolidine-3-carboxylate

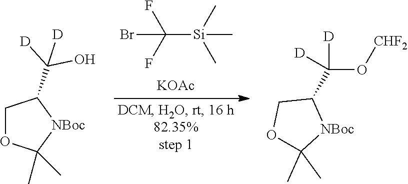

To a stirred solution of tert-butyl (4R)-4-[hydroxy(2H2)methyl]-2,2-dimethyl-1,3-oxazolidine-3-carboxylate (20 g, 85.837 mmol, 1 equiv) and (bromodifluoromethyl)trimethylsilane (52.232 g, 257.175 mmol, 3 equiv) in DCM (800.00 mL) and H₂O (800.00 mL) was added potassium acetate (50.479 g, 514.350 mmol, 6 equiv) in portions at 10° C. under air atmosphere. The resulting mixture was stirred for 16 h at room temperature under air atmosphere. The resulting mixture was concentrated under reduced pressure. The resulting mixture was extracted with CH₂Cl₂ (3×10 mL). The combined organic layers were washed with brine (1×5 mL), dried over anhydrous Na₂SO₄. After filtration, the filtrate was concentrated under reduced pressure. The residue was purified by silica gel column chromatography, eluted with PE/EA (4:1) to afford tert-butyl (4R)-4-[(difluoromethoxy)(2H2)methyl]-2,2-dimethyl-1,3-oxazolidine-3-carboxylate (20 g, 82.35%) as a yellow oil.

### Step 2. Synthesis of tert-butyl N-[(2R)-1-(difluoromethoxy)-3-hydroxy(1,1-2H2)propan-2-yl]carbamate

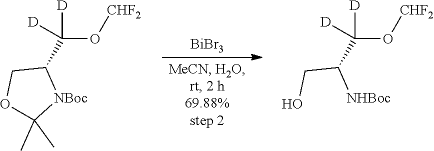 [SMILES: [2H]C([2H])(OC(F)F)[C@H]1COC(C)(C)N1C(=O)OC(C)(C)C ; "BiBr3" ; [2H]C([2H])(OC(F)F)[C@@H](CO)NC(=O)OC(C)(C)C]

To a stirred solution of tert-butyl (4R)-4-[(difluoromethoxy)(2H2)methyl]-2,2-dimethyl-1,3-oxazolidine-3-carboxylate (4 g, 14.119 mmol, 1 equiv) in MeCN (80 mL) was added bismuth tribromide (1.27 g, 2.824 mmol, 0.2 equiv) dropwise at 0° C. under air atmosphere. The resulting mixture was stirred for 2 h at room temperature under air atmosphere. The reaction was quenched with Water (20 mL) at room temperature. The resulting mixture was filtered, the filter cake was washed with MeCN (1×20 mL). The filtrate was concentrated under reduced pressure. The residue was purified by silica gel column chromatography, eluted with PE/EA (4:1) to afford tert-butyl N-[(2R)-1-(difluoromethoxy)-3-hydroxy(1,1-2H2)propan-2-yl]carbamate (2.4 g, 69.88%) as a yellow oil.

Synthesis of tert-butyl (R)-4-(fluoromethyl-d2)-1,2,3-oxathiazolidine-3-carboxylate 2,2-dioxide (AA-46) was performed according to the general method starting from tert-butyl N-[(2R)-1-fluoro-3-hydroxy(1,1-2H2)propan-2-yl]carbamate.

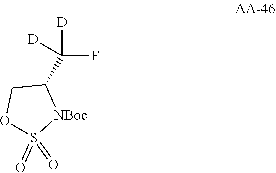 [SMILES: [2H]C([2H])(F)[C@H]1COS(=O)(=O)N1C(=O)OC(C)(C)C]

### Synthesis of tert-butyl N-[(2R)-1-fluoro-3-hydroxy(1,1-2H2)propan-2-yl]carbamate

### Step 1 tert-butyl (4R)-2,2-dimethyl-4-{[(4-methylbenzenesulfonyl)oxy](2H2)methyl}-1,3-oxazolidine-3-carboxylate

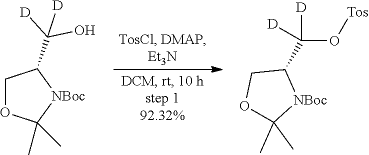

To a stirred solution of tert-butyl (4R)-4-[hydroxy(2H2)methyl]-2,2-dimethyl-1,3-oxazolidine-3-carboxylate (3 g, 12.859 mmol, 1 equiv) in DCM (40 mL) were added 4-methylbenzene-1-sulfonyl chloride (3.68 g, 19.288 mmol, 1.5 equiv) and Et₃N (2.60 g, 25.718 mmol, 2 equiv) in portions at 0° C. under nitrogen atmosphere. The resulting mixture was stirred for overnight at room temperature under nitrogen atmosphere. The resulting mixture was diluted with water (10 mL). The resulting mixture was extracted with CH₂Cl₂ (3×50 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (4:1) to afford tert-butyl (4R)-2,2-dimethyl-4-{[(4-methylbenzenesulfonyl)oxy](2H2)methyl}-1,3-oxazolidine-3-carboxylate (4.6 g, 92.32%) as a white solid.

### Step 2. Synthesis of tert-butyl (4R)-4-[fluoro(2H2)methyl]-2,2-dimethyl-1,3-oxazolidine-3-carboxylate

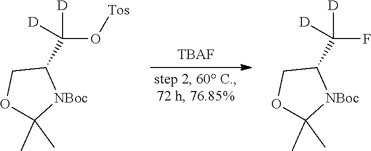

Into a 100 mL 3-necked round-bottom flask were added tert-butyl (4R)-2,2-dimethyl-4-{[(4-methylbenzenesulfonyl)oxy](2H2)methyl}-1,3-oxazolidine-3-carboxylate (3 g, 7.742 mmol, 1 equiv) and TBAF (4.45 g, 17.032 mmol, 2.2 equiv) at room temperature. The resulting mixture was stirred for 3 days at 60° C. under air atmosphere. The mixture was allowed to cool down to room temperature. The resulting mixture was diluted with EtOAc (60 mL). The resulting mixture was extracted with EtOAc (3×50 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (9:1) to afford tert-butyl (4R)-4-[fluoro(2H2)methyl]-2,2-dimethyl-1,3-oxazolidine-3-carboxylate (1.4 g, 76.85%) as a yellow oil.

### Step 3. Synthesis of tert-butyl N-[(2R)-1-fluoro-3-hydroxy(1,1-2H2)propan-2-yl]carbamate

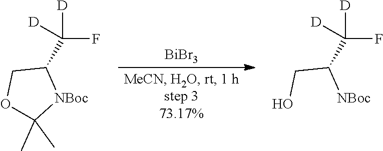

To a stirred solution of tert-butyl (4R)-4-[fluoro(2H2)methyl]-2,2-dimethyl-1,3-oxazolidine-3-carboxylate (1.4 g, 5.950 mmol, 1 equiv) in MeCN (20 mL) was added bismuth tribromide (0.53 g, 1.190 mmol, 0.2 equiv) and H₂O (0.2 mL) dropwise at 0° C. under air atmosphere. The resulting mixture was stirred for 1 h at room temperature under air atmosphere. The resulting mixture was extracted with CH₂Cl₂ (3×30 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (4:1) to afford tert-butyl N-[(2R)-1-fluoro-3-hydroxy(1,1-2H2)propan-2-yl]carbamate (850 mg, 73.17%).

Synthesis of tert-butyl (R)-4-(fluoromethyl)-1,2,3-oxathiazolidine-3-carboxylate-5,5-d2 2,2-dioxide (AA-47) was performed according to the general method starting from tert-butyl N-[(2R)-1-fluoro-3-hydroxy(3,3-2H2)propan-2-yl]carbamate.

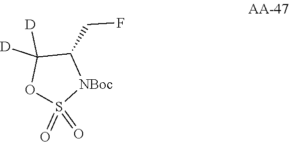 [SMILES: [2H]C1([2H])OS(=O)(=O)N(C(=O)OC(C)(C)C)[C@H]1CF]

### Synthesis of tert-butyl N-[(2R)-1-fluoro-3-hydroxy(3,3-2H2)propan-2-yl]carbamate

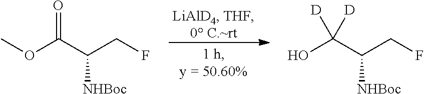

Into a 500-mL 3-necked round-bottom as were added methyl 2R-2-[(tert-butoxycarbonyl)amino]-3-fluoropropanoate (21.5 g, 97.185 mmol, 1.00 equiv) and THF (200 mL) at 0° C. To the above mixture was added LiAlD₄ (4.90 g, 116.722 mmol, 1.20 equiv) in portions over 30 min at room temperature. The resulting mixture was stirred for additional 1 h at room temperature. The resulting mixture was diluted with THF (200 mL). The mixture was allowed to cool down to 0° C. The reaction was quenched by the addition of Water (86 mL) and 15% NaOH (21.5 mL) at 0° C. The residue was purified by silica gel column chromatography, eluted with PE/EA (4:1) to afford tert-butyl N-[(2R)-1-fluoro-3-hydroxy(3,3-2H2)propan-2-yl]carbamate (9.6 g, 50.60%) as a colorless oil.

Reference for tert-butyl (S)-4-(methoxymethyl)-1,2,3-oxathiazolidine-3-carboxylate-5,5-d2 2,2-dioxide (AA-48): (WO2022/89454)

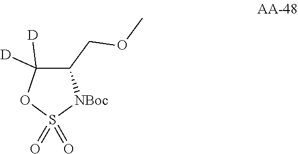

Aldehydes Used: AL-1: (S)—N-Boc-2-aminopropanal

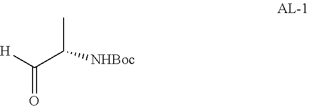

AL-2: Tert-butyl (S)-4-formyl-2,2-dimethyloxazolidine-3-carboxylate

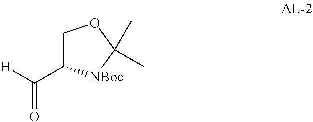 [SMILES: CC(C)(C)OC(=O)N1[C@H](C=O)COC1(C)C]

AL-3: Tert-butyl (S)-(1-methoxy-3-oxopropan-2-yl)carbamate

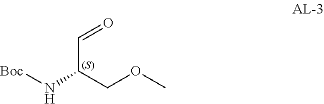

### Additional Building Blocks:

### Synthesis of 3-bromo-5-chloro-7-iodofuro[3,2-b]pyridine

### Step 1. Synthesis of 6-chloro-2-iodopyridin-3-ol

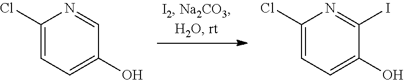 [SMILES: Oc1ccc(Cl)nc1 ; Oc1ccc(Cl)nc1I]

To a stirred solution of 6-chloropyridin-3-ol (100 g, 1 equiv, 770 mmol) in water (1400 mL) was added iodide (196 g, 1 equiv, 770 mmol) and sodium carbonate (164 g, 2 equiv, 1500 mmol). This mixture was stirred at 25° C. for 3 h. The pH value of the solution was adjusted to 6-7 with hydrochloric acid (1 mol/L, 900 mL). The resulting solution was extracted with ethyl acetate (3×1500 mL). The combined organic layers were washed with sat. sodium chloride aqueous (3×1500 mL), dried over anhydrous sodium sulfate. After filtration, the filtrate was concentrated under reduced pressure to afford 6-chloro-2-iodopyridin-3-ol (183 g, 92%) as a yellow solid.

### Step 2. Synthesis of 6-chloro-2-((triisopropylsilyl)ethynyl)pyridin-3-ol

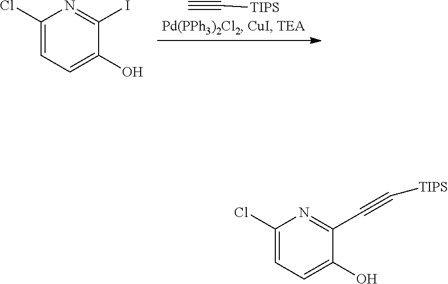

To a stirred solution of 6-chloro-2-iodopyridin-3-ol (180 g, 1 equiv, 700 mmol) in 1,4-dioxane (1200 mL) and triethylamine (1200 mL) was added ethynyltriisopropylsilane (167 g, 1.3 equiv, 910 mmol), cuprous iodide (6.71 g, 0.05 equiv, 35 mmol) and bis-(triphenylphosphino)-palladous chloride (9.89 g, 0.02 equiv, 14 mmol). This mixture was stirred at 45° C. for 2.5 h under nitrogen atmosphere. After the reaction was cooled to room temperature, the residue was diluted with water (2000 mL) and extracted with ethyl acetate (3×1500 mL). The combined organic layers were dried over anhydrous sodium sulfate. After filtration, the filtrate was concentrated under reduced pressure. The residue was purified by silica gel column chromatography, eluted with petroleum ether/ethyl acetate (5:1) to afford 6-chloro-2-((triisopropylsilyl)ethynyl)pyridin-3-ol (162 g, 73%) as a yellow solid.

### Step 3. Synthesis of 5-chloro-2-(triisopropylsilyl)furo[3,2-b]pyridine

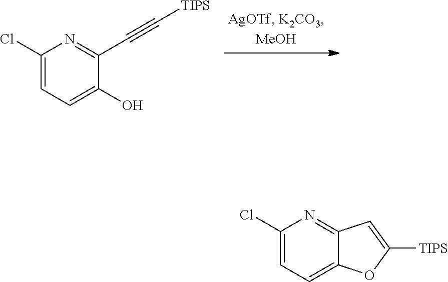

To a stirred solution of 6-chloro-2-((triisopropylsilyl)ethynyl)pyridin-3-ol (160 g, 1 equiv, 510 mmol) in methanol (1600 mL) was added potassium carbonate (107 g, 1.5 equiv, 770 mmol) and silver(I)trifluoromethanesulfonate (13.3 g, 0.1 equiv, 51 mmol). This mixture was stirred at 50° C. for 12 h. After the reaction was cooled to room temperature, the reaction was filtered and the filtrate was concentrated under reduced pressure. The residue was purified by silica gel column chromatography, eluted with petroleum ether/ethyl acetate (10:1) to afford 5-chloro-2-(triisopropylsilyl)furo[3,2-b]pyridine (150 g, 93%) as a yellow solid.

### Step 4. Synthesis of 5-chloro-7-iodo-2-(triisopropylsilyl)furo[3,2-b]pyridine

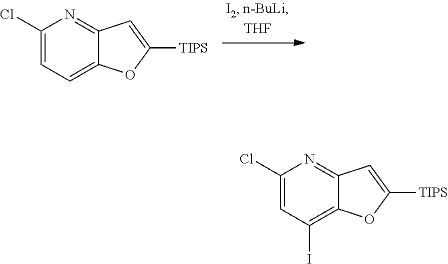

To a solution of 5-chloro-2-(triisopropylsilyl)furo[3,2-b]pyridine (70 g, 1 equiv, 0.22 mol) in tetrahydrofuran (500 mL) was added n-butyllithium (136 mL, 2 mol/L, 1.2 equiv, 0.28 mol) dropwise and the resulting mixture was stirred at −70° C. for 40 minutes. A solution of iodide (86 g, 1.5 equiv, 0.34 mol) in tetrahydrofuran (240 mL) was added dropwise and the resulting mixture was allowed to warm up to 0° C. over 3 h. The saturated ammonium chloride aqueous (600 mL) and saturated sodium thiosulfate aqueous solution (400 mL) were added at 0° C. The mixture was extracted with dichloromethane (3×700 mL) and the combined organic layers were dried over anhydrous sodium sulfate. After filtration, the filtrate was concentrated under reduced pressure. The residue was purified by silica gel column chromatography, eluted with petroleum ether/ethyl acetate (10:1) to afford 5-chloro-7-iodo-2-(triisopropylsilyl)furo[3,2-b]pyridine (74 g, 75%) as a yellow solid.

### Step 5. Synthesis of 5-chloro-7-iodofuro[3,2-b]pyridine

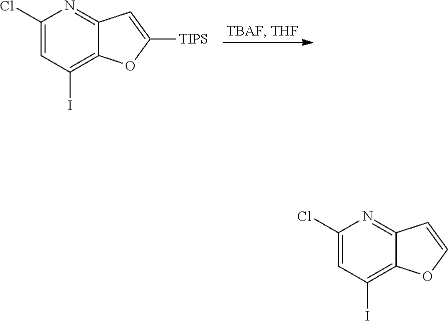

To a solution of 5-chloro-7-iodo-2-(triisopropylsilyl)furo[3,2-b]pyridine (74 g, 1 equiv, 170 mmol) in tetrahydrofuran (400 mL) was added tetrabutylammonium fluoride (204 mL, 1 mol/L in THf, 1.2 equiv, 0.20 mol) dropwise at −45° C. and the resulting mixture was allowed to warm to 0° C. over 60 minutes. The saturated ammonium chloride (120 mL) aqueous was added at 0° C. The mixture was extracted with ethyl acetate (3×700 mL) and the combined organic layers were dried over anhydrous sodium sulfate. After filtration, the filtrate was concentrated under reduced pressure. The residue was triturated by isopropyl alcohol and filtered to afford 5-chloro-7-iodofuro[3,2-b]pyridine (46 g, 97%) as a white solid.

### Step 6. Synthesis of 3-bromo-5-chloro-7-iodofuro[3,2-b]pyridine

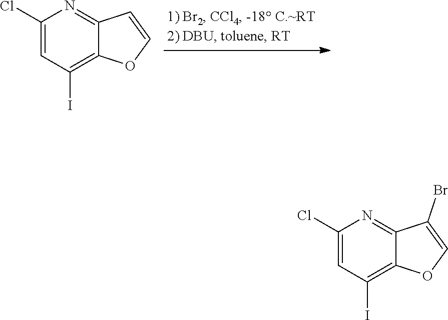 [SMILES: Clc1cc(I)c2occc2n1 ; Clc1cc(I)c2occ(Br)c2n1]

To a solution of 5-chloro-7-iodofuro[3,2-b]pyridine (44 g, 1 equiv, 158 mmol) in CCl₄ (480 mL) was added bromine (0.38 kg, 122 mL, 15 equiv, 2.4 mol) dropwise at −18° C. and the resulting mixture was allowed to warm to 25° C. over 1.5 h. Then the mixture was poured into saturated sodium thiosulfate aqueous (1000 mL) at 0° C. and filtered through a Celite pad. The resulting mixture was extracted with dichloromethane (2×2000 mL). The organic layers were washed with brine (2000 mL) and dried over anhydrous sodium sulfate. After filtration, the filtrate was concentrated under reduced pressure. Toluene (500 mL) and 1,8-Diazabicyclo[5.4.0]undec-7-ene (80 mL) were added to the residue and the mixture was stirred at 25° C. for 45 minutes. The mixture was concentrated under reduced pressure and purified by silica gel column chromatography, eluted with petroleum ether/ethyl acetate (7:1) to afford 3-bromo-5-chloro-7-iodofuro[3,2-b]pyridine (24 g, 43%) as a white solid.

### Synthesis of 3-bromo-5-chloro-7-(methylsulfanyl)furo[3,2-b]pyridine

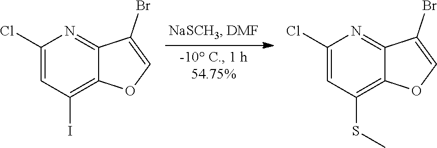

To a stirred mixture of 3-bromo-5,7-dichlorofuro[3,2-b]pyridine (2.1 g, 7.868 mmol, 1 equiv) in DMF (20 mL) was added (methylsulfanyl)sodium (551.40 mg, 7.868 mmol, 1 equiv) in portions at −10° C. under nitrogen atmosphere. The resulting mixture was stirred for 1 h at −10° C. under nitrogen atmosphere. The residue was purified by reverse flash chromatography with the following conditions: column, C₁₈ silica gel; mobile phase, MeCN in Water (10 mmol/L NH₄HCO₃), 10% to 60% gradient in 15 min; detector, UV 254 nm. This resulted in 3-bromo-5-chloro-7-(methylsulfanyl)furo[3,2-b]pyridine (1.2 g, 54.75%) as a white solid.

### Synthesis of 5-chloro-7-(methylsulfanyl)furo[3,2-b]pyridin-3-ylboronic acid

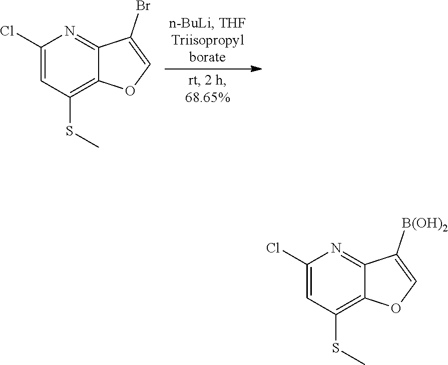

A solution of 3-bromo-5-chloro-7-(methylsulfanyl)furo[3,2-b]pyridine (500 mg, 1.795 mmol, 1 equiv) in THE (10 mL) was treated with n-BuLi (0.93 mL, 2.333 mmol, 1.3 equiv) for 5 min at −78° C. under nitrogen atmosphere followed by the addition of triisopropyl borate (506.39 mg, 2.692 mmol, 1.5 equiv) dropwise at −78° C. The resulting mixture was stirred for additional 2 h at room temperature. The residue was purified by silica gel column chromatography, eluted with CH₂Cl₂/MeOH (10:1) to afford 5-chloro-7-(methylsulfanyl)furo[3,2-b]pyridin-3-ylboronic acid (300 mg, 68.65%) as a white solid.

### Synthesis of 3,5-dichloro-7-(methylsulfanyl)furo[3,2-b]pyridine

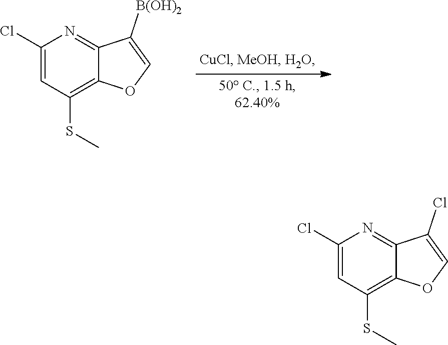

To a stirred solution of 5-chloro-7-(methylsulfanyl)furo[3,2-b]pyridin-3-ylboronic acid (500 mg, 2.054 mmol, 1 equiv) and CuCl (609.93 mg, 6.162 mmol, 3 equiv) in MeOH (6 mL) and H₂O (2 mL) under nitrogen atmosphere. The resulting mixture was stirred for additional 1.5 h at 50° C. The residue was purified by reversed-phase flash chromatography with the following conditions: (column, C₁₈; mobile phase, MeCN in Water (0.1% FA), 10% to 100% gradient in 10 min; detector, UV 254 nm.) to afford 3,5-dichloro-7-(methylsulfanyl)furo[3,2-b]pyridine (300 mg, 62.40%) as a light yellow solid.

### Synthesis of 3-bromo-5-chloro-6-fluoro-7-iodofuro[3,2-b]pyridine

### Step 1. Synthesis of 6-chloro-5-fluoropyridin-3-ol

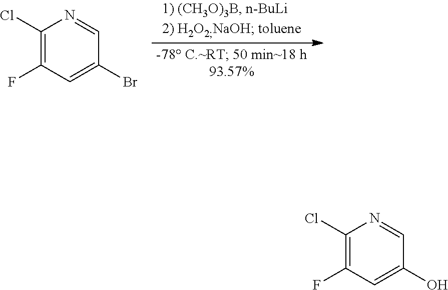 [SMILES: Fc1cc(Br)cnc1Cl ; Oc1cnc(Cl)c(F)c1]

To a stirred solution of 5-bromo-2-chloro-3-fluoropyridine (50 g, 237.609 mmol, 1 equiv) in toluene (500 mL) was added n-BuLi (2.5 M in hexane, 104.5 mL, 1631.283 mmol, 1.1 equiv) dropwise at −78° C. under nitrogen atmosphere. The resulting mixture was stirred for 10 min at −78° C. under nitrogen atmosphere. To the above mixture was added trimethyl borate (29.63 g, 285.131 mmol, 1.2 equiv) dropwise over 20 min at −78° C. The resulting mixture was stirred for additional 20 min at −78° C. The resulting mixture was stirred for overnight at room temperature under nitrogen atmosphere. Into the 2 L 4-necked round-bottom flask were added 8N sat. NaOH (aq.) (337 mL, 2696.000 mmol, 11.35 equiv) and H₂O₂ (30%) (225 mL, 9657.788 mmol, 40.65 equiv) at 0° C. The resulting mixture was stirred for 2 h at room temperature under air atmosphere. The reaction was quenched with sat. Na₂S₂O₃ (aq.) (500 mL) at 0° C. The aqueous layer was extracted with EtOAc (3×1000 mL). The combined organic layers were washed with water/NaCl (1×1000 mL), dried over anhydrous Na₂SO₄. After filtration, the filtrate was concentrated under reduced pressure. This resulted in 6-chloro-5-fluoropyridin-3-ol (32.8 g, 93.57%) as a yellow solid.

### Step 2. Synthesis of 6-chloro-5-fluoro-2-iodopyridin-3-ol

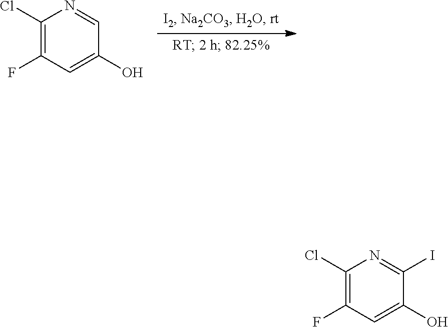 [SMILES: Oc1cnc(Cl)c(F)c1 ; Oc1cc(F)c(Cl)nc1I]

To a stirred solution of 6-chloro-5-fluoropyridin-3-ol (32.8 g, 222.328 mmol, 1 equiv) and Na₂CO₃ (47.13 g, 444.656 mmol, 2 equiv) in water (400 mL) was added I₂ (59.25 g, 233.444 mmol, 1.05 equiv) in portions at room temperature under air atmosphere. The resulting mixture was stirred for 2 h at room temperature under air atmosphere. The reaction was quenched with sat. Na₂S₂O₃ (aq.) (100 mL) at room temperature. The aqueous layer was extracted with EtOAc (3×500 mL). The resulting mixture was concentrated under vacuum. This resulted in 6-chloro-5-fluoro-2-iodopyridin-3-ol (50 g, 82.25%) as a yellow solid.

### Step 3. Synthesis of 6-chloro-5-fluoro-2-[2-(triisopropylsilyl)ethynyl]pyridin-3-ol

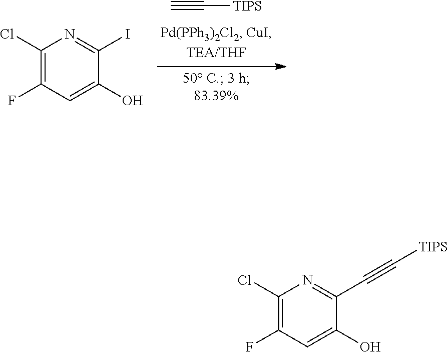

To a stirred solution of 6-chloro-5-fluoro-2-iodopyridin-3-ol (50 g, 182.862 mmol, 1 equiv) and ethynyltriisopropylsilane (41.69 g, 228.577 mmol, 1.25 equiv) in THF (150 mL) and TEA (150 mL) were added CuI (1.39 g, 7.314 mmol, 0.04 equiv) and Pd(PPh₃)₂Cl₂ (2.57 g, 3.657 mmol, 0.02 equiv) under nitrogen atmosphere. The resulting mixture was stirred for 3 h at 50° C. under nitrogen atmosphere. The reaction was quenched by the addition of Water (200 mL) at room temperature. The resulting mixture was extracted with EtOAc (3×200 mL). dried over anhydrous Na₂SO₄. After filtration, the filtrate was concentrated under reduced pressure. This resulted in 6-chloro-5-fluoro-2-[2-(triisopropylsilyl)ethynyl]pyridin-3-ol (50 g, 83.39%) as a black solid.

### Step 4. Synthesis of 5-chloro-6-fluoro-2-(triisopropylsilyl)furo[3,2-b]pyridine

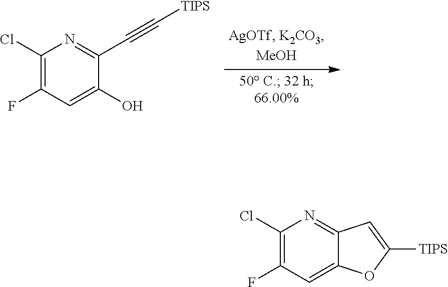

To a stirred solution of 6-chloro-5-fluoro-2-[2-(triisopropylsilyl)ethynyl]pyridin-3-ol (50 g, 152.486 mmol, 1 equiv) and K₂CO₃ (94.83 g, 686.187 mmol, 4.5 equiv) in MeOH (250 mL) was added silver(1+) trifluoromethanesulfonate (15.67 g, 60.994 mmol, 0.4 equiv) at 50° C. under nitrogen atmosphere. The resulting mixture was stirred for 2 days at 50° C. under nitrogen atmosphere. The reaction was quenched with Water at room temperature. The resulting mixture was concentrated under vacuum. The resulting mixture was extracted with EtOAc (3×200 mL). After filtration, the filtrate was concentrated under reduced pressure. The residue was purified by silica gel column chromatography, eluted with PE/EA (12:1) to afford 5-chloro-6-fluoro-2-(triisopropylsilyl)furo[3,2-b]pyridine (33 g, 66.00%) as a brown solid.

### Step 5. Synthesis of 5-chloro-6-fluoro-7-iodo-2-(triisopropylsilyl)furo[3,2-b]pyridine

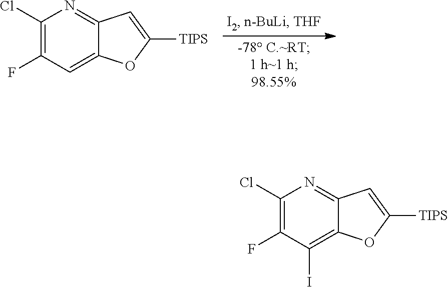

A solution of 5-chloro-6-fluoro-2-(triisopropylsilyl)furo[3,2-b]pyridine (5 g, 15.249 mmol, 1 equiv) in THE (30 mL) was treated with n-BuLi 2.5M in hexanes (9.75 mL, 1.6 equiv) for 60 min dropwise at −78° C. under nitrogen atmosphere followed by the addition of I₂ (5.03 g, 19.824 mmol, 1.3 equiv) in THE (30 mL) dropwise at −78° C. The resulting mixture was stirred for 1 h at −50° C. under nitrogen atmosphere. The reaction was quenched with sat. Na₂S₂O₃ (aq.) (30 mL) at room temperature. The aqueous layer was extracted with EtOAc (4×30 mL). The resulting mixture was concentrated under vacuum. This resulted in 5-chloro-6-fluoro-7-iodo-2-(triisopropylsilyl)furo[3,2-b]pyridine (6.75 g, 97.55%) as a yellow solid.

### Step 6. Synthesis of 5-chloro-6-fluoro-7-iodofuro[3,2-b]pyridine

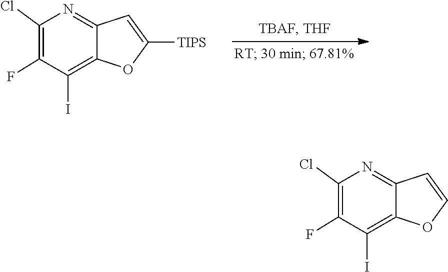

To a stirred solution of 5-chloro-6-fluoro-7-iodo-2-(triisopropylsilyl)furo[3,2-b]pyridine (6.75 g, 14.874 mmol, 1 equiv) in THE (50 mL) was added TBAF 1M in THE (4.45 mL, 0.3 equiv) dropwise at 0° C. under air atmosphere. The resulting mixture was stirred for 0.5 h at room temperature under air atmosphere. The reaction was quenched with Water at room temperature. The aqueous layer was extracted with EtOAc (3×50 mL). The resulting mixture was concentrated under vacuum. The residue was purified by silica gel column chromatography, eluted with PE/EA (20:1) to afford 5-chloro-6-fluoro-7-iodofuro[3,2-b]pyridine (3 g, 67.81%) as a yellow solid.

### Step 7. Synthesis of 3-bromo-5-chloro-6-fluoro-7-iodofuro[3,2-b]pyridine

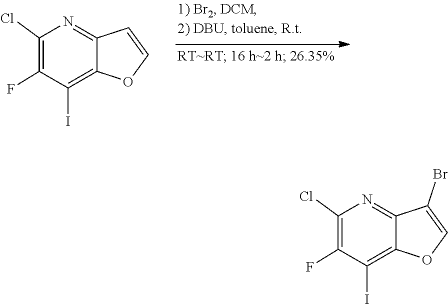 [SMILES: Fc1c(Cl)nc2ccoc2c1I ; Fc1c(Cl)nc2c(Br)coc2c1I]

Into a 40 mL vial were added 5-chloro-6-fluoro-7-iodofuro[3,2-b]pyridine (3 g, 10.086 mmol, 1 equiv) and DCM (30.00 mL). Then Br₂ (8058.90 mg, 50.430 mmol, 5 equiv) were added at 0° C. The resulting mixture was stirred for overnight at room temperature under nitrogen atmosphere. The reaction was quenched with sat. Na₂S₂O₃ (aq.) (10 mL) at room temperature. The aqueous layer was extracted with CH₂Cl₂ (3×30 mL). The resulting mixture was concentrated under reduced pressure. Then the crude product was dissolved in THF (30.00 mL) and DBU (3.0 g, 20.172 mmol, 2 equiv) was added. After stirring for 2 h at room temperature, the resulting mixture was concentrated under reduced pressure. The residue was purified by silica gel column chromatography, eluted with PE/EA (9:1) to afford 3-bromo-5-chloro-6-fluoro-7-iodofuro[3,2-b]pyridine (1 g, 26.35%) as a white solid.

### Synthesis of 5-fluoro-7-iodo-3-methylfuro[3,2-b]pyridine

### Step 1. Synthesis of 6-fluoro-2-iodopyridin-3-ol

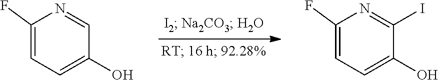 [SMILES: Oc1ccc(F)nc1 ; Oc1ccc(F)nc1I]

To a stirred solution of 6-fluoropyridin-3-ol (20 g, 176.849 mmol, 1 equiv) in THF (600.0 mL) was added Na₂CO₃ (37.49 g, 353.698 mmol, 2 equiv) in H₂O (600.0 g) at 0° C. and stirred for 30 min. I₂ (44.89 g, 176.849 mmol, 1 equiv) was added and the mixture was allowed to warm to RT and stirred for overnight. The resulting mixture was concentrated under reduced pressure and acidified to pH 7 with 1N HCl (aq.). The aqueous layer was extracted with EtOAc (3×500 mL). The residue was purified by silica gel column chromatography, eluted with PE/EA (4:1) to afford 6-fluoro-2-iodopyridin-3-ol (39 g, 92.28%) as a yellow solid.

### Step 2. Synthesis of 6-fluoro-2-iodopyridin-3-yl acetate

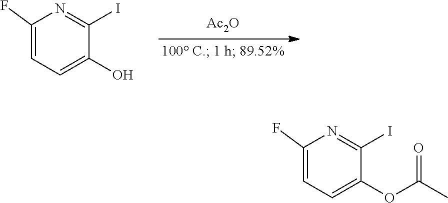

Into a 1 L round-bottom flask were added 6-fluoro-2-iodopyridin-3-ol (38 g, 159.004 mmol, 1 equiv) and acetic anhydride (200 mL) at room temperature. The resulting mixture was stirred for 1 h at 100° C. The reaction was quenched with sat. NaHCO₃ (aq.) at room temperature. The aqueous layer was extracted with EtOAc (3×500 mL). The residue was purified by silica gel column chromatography, eluted with PE/EA (5:1) to afford 6-fluoro-2-iodopyridin-3-yl acetate (40 g, 89.52%) as a yellow oil.

### Step 3. Synthesis of 6-fluoro-2-((triisopropylsilyl)ethynyl)pyridin-3-ol

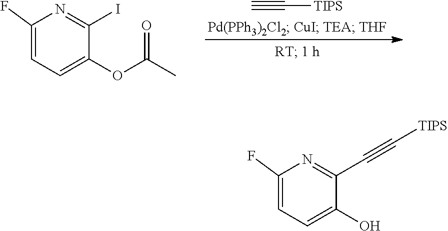

To a solution of 6-fluoro-2-iodopyridin-3-yl acetate (16 g, 56.934 mmol, 1 equiv) and ethynyltriisopropylsilane (10.59 g, 58.073 mmol, 1.02 equiv) in THE (160 mL) and TEA (50 mL) were added Pd(PPh₃)₂Cl₂ (799.25 mg, 1.139 mmol, 0.02 equiv) and CuI (433.73 mg, 2.277 mmol, 0.04 equiv). After stirring for 1 h at room temperature under a nitrogen atmosphere, the reaction was quenched with water (500 mL) at room temperature. The resulting mixture was extracted with EtOAc (2×300 mL). The combined organic layers were washed with brine (2×200 mL), dried over anhydrous Na₂SO₄. After filtration, the filtrate was concentrated under reduced pressure. The crude product was used in the next step directly without further purification.

### Step 4. Synthesis of 5-fluoro-3-iodo-2-(triisopropylsilyl)furo[3,2-b]pyridine

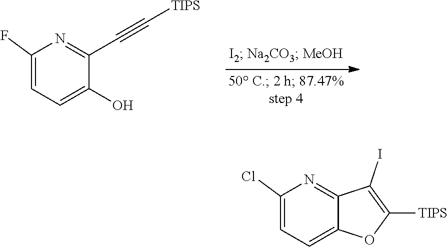

To a solution of 6-fluoro-2-[2-(triisopropylsilyl)ethynyl]pyridin-3-ol (crude) and Cs₂CO₃ (53.29 g, 163.566 mmol, 3 equiv) in MeOH (250 mL) were added I₂ (41.51 g, 163.566 mmol, 3 equiv). After stirring for 2 h at 50° C., the reaction was quenched by the addition of sat. Na₂S₂O₃ (aq.) (300 mL) at room temperature. The resulting mixture was concentrated under reduced pressure. The aqueous layer was extracted with EtOAc (3×300 mL). The residue was purified by silica gel column chromatography, eluted with PE/EA (50:1) to afford 5-fluoro-3-iodo-2-(triisopropylsilyl)furo[3,2-b]pyridine (20 g, 87.47%) as a yellow solid.

### Step 5. Synthesis of 5-fluoro-3-methyl-2-(triisopropylsilyl)furo[3,2-b]pyridine

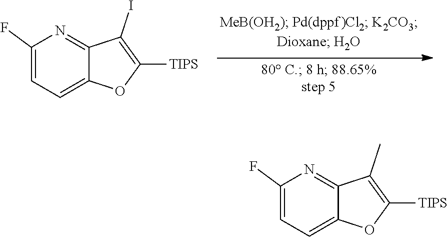

To a solution of 5-fluoro-3-iodo-2-(triisopropylsilyl)furo[3,2-b]pyridine (20 g, 47.692 mmol, 1 equiv) and methylboronic acid (8.56 g, 143.076 mmol, 3 equiv) in dioxane (200 mL) and H₂O (20 mL) were added K₂CO₃ (32.96 g, 238.460 mmol, 5 equiv) and Pd(dppf)Cl₂ (6.98 g, 9.538 mmol, 0.2 equiv). After stirring for 8 h at 80° C. under a nitrogen atmosphere, the reaction was quenched with water (300 mL) at room temperature. The resulting mixture was extracted with EtOAc (2×300 mL). The residue was purified by silica gel column chromatography, eluted with PE/EA (30:1) to afford 5-fluoro-3-methyl-2-(triisopropylsilyl)furo[3,2-b]pyridine (13 g, 88.65%) as a yellow oil.

### Step 6. Synthesis of 5-fluoro-7-iodo-3-methyl-2-(triisopropylsilyl)furo[3,2-b]pyridine

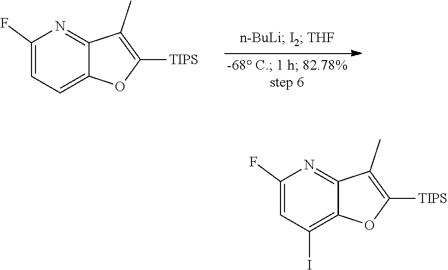

A solution of 5-fluoro-3-methyl-2-(triisopropylsilyl)furo[3,2-b]pyridine (6 g, 19.513 mmol, 1 equiv) in THE (200 mL) was treated with n-BuLi (2.5 M in hexane, 16.39 mL, 40.977 mmol, 2.1 equiv) for 1 h at −68° C. under nitrogen atmosphere followed by the addition of I₂ (7.92 g, 31.221 mmol, 1.6 equiv) in 20 mL THE dropwise at −68° C. The mixture was stirred for 1 h at −68° C. under nitrogen atmosphere. The reaction was quenched by the addition of water (100 mL) at room temperature. The resulting mixture was extracted with EtOAc (3×300 mL). The combined organic layers were dried over anhydrous Na₂SO₄. After filtration, the filtrate was concentrated under reduced pressure. The residue was purified by silica gel column chromatography, eluted with PE/EA (50:1) to afford 5-fluoro-7-iodo-3-methyl-2-(triisopropylsilyl)furo[3,2-b]pyridine (7 g, 82.78%) as a yellow solid.

### Step 7. Synthesis of 5-fluoro-7-iodo-3-methylfuro[3,2-b]pyridine

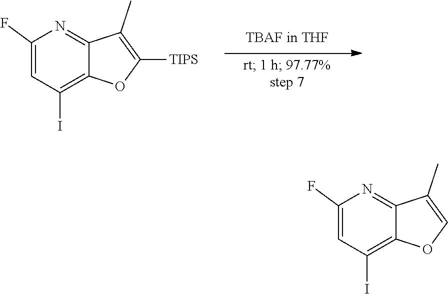

A solution of 5-fluoro-7-iodo-3-methyl-2-(triisopropylsilyl)furo[3,2-b]pyridine (8 g, 18.460 mmol, 1 equiv) in THE (80 mL) was treated with TBAF (1M in THF) (9.22 mL, 9.230 mmol, 0.5 equiv) for 20 min at 0° C. The mixture was stirred for 1 h at room temperature. The reaction was quenched by the addition of water (100 mL) at room temperature. The resulting mixture was extracted with EtOAc (3×100 mL). The combined organic layers were dried over anhydrous Na₂SO₄. After filtration, the filtrate was concentrated under reduced pressure. The residue was purified by silica gel column chromatography, eluted with PE/EA (12:1) to afford 5-fluoro-7-iodo-3-methylfuro[3,2-b]pyridine (5 g, 97.77%) as a yellow solid.

### Synthesis of 1,3-dioxoisoindol-2-yl 2-[(tert-butoxycarbonyl)amino]-4,4-difluorocyclopentane-1-carboxylate

### Step 1. Synthesis of 1,2-dimethyl 4,4-difluorocyclopentane-1,2-dicarboxylate

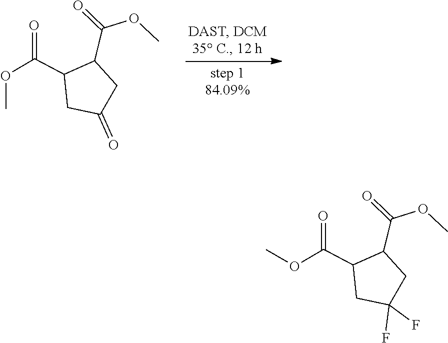

To a stirred solution of 1,2-dimethyl 4-oxocyclopentane-1,2-dicarboxylate (15 g, 74.929 mmol, 1 equiv) in DCM (150 mL) was added DAST (30.19 g, 187.322 mmol, 2.5 equiv) dropwise at 0° C. under nitrogen atmosphere. The resulting mixture was stirred for 12 h at 35° C. under nitrogen atmosphere. The reaction was quenched with Water (400 mL) at room temperature. The mixture was basified to pH=8 with saturated NaHCO₃ (aq.). The resulting mixture was extracted with CH₂Cl₂ (3×200 mL). The combined organic layers were dried over anhydrous Na₂SO₄. After filtration, the filtrate was concentrated under reduced pressure. The residue was purified by silica gel column chromatography, eluted with PE/EA (10:1) to afford 1,2-dimethyl 4,4-difluorocyclopentane-1,2-dicarboxylate (14 g, 84.09%) as a light yellow solid.

### Step 2. Synthesis of 4,4-difluoro-2-(methoxycarbonyl)cyclopentane-1-carboxylic acid

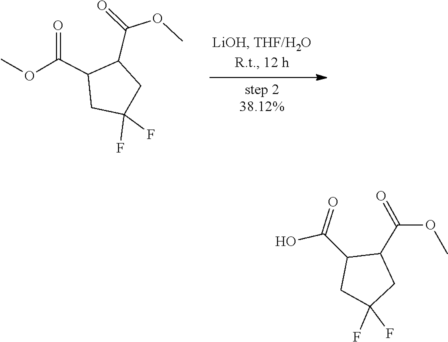

To a stirred solution of 1,2-dimethyl 4,4-difluorocyclopentane-1,2-dicarboxylate (14 g, 63.010 mmol, 1 equiv) in THE (140 mL) and H₂O (40 mL) was added LiOH (1.66 g, 69.311 mmol, 1.1 equiv) at 0° C. The resulting mixture was stirred for 12 h at room temperature under nitrogen atmosphere. The mixture was acidified to pH=4 with HCl (aq.). The resulting mixture was extracted with CH₂Cl₂ (3×30 mL). The combined organic layers were dried over anhydrous Na₂SO₄. After filtration, the filtrate was concentrated to afford 4,4-difluoro-2-(methoxycarbonyl)cyclopentane-1-carboxylic acid (5 g, 38.12%) as a light yellow oil.

### Step 3. Synthesis of methyl 2-{[(benzyloxy)carbonyl]amino}-4,4-difluorocyclopentane-1-carboxylate

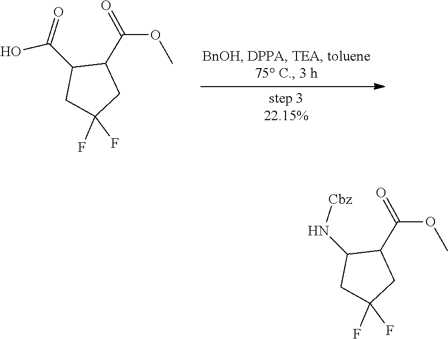 [SMILES: COC(=O)C1CC(F)(F)CC1C(=O)O ; COC(=O)C1CC(F)(F)CC1NC(=O)OCc1ccccc1]

A mixture of 4,4-difluoro-2-(methoxycarbonyl)cyclopentane-1-carboxylic acid (3 g, 14.412 mmol, 1 equiv), TEA (2916.77 mg, 28.824 mmol, 2 equiv) and DPPA (4759.46 mg, 17.294 mmol, 1.2 equiv) in Toluene (30 mL) was stirred for 15 min at 75° C. under nitrogen atmosphere. To the above mixture was added benzyl alcohol (10 mL) dropwise over at 75° C. The resulting mixture was stirred for additional 3 h at 75° C. The resulting mixture was washed with 2×30 mL of brine. The resulting mixture was concentrated under reduced pressure. The residue was purified by reversed-phase flash chromatography with the following conditions: column, C₁₈; mobile phase, MeCN in Water (10 mmol/L NH₄HCO₃), 20% to 100% gradient in 15 min; detector, UV 254 nm. This resulted in methyl 2-{[(benzyloxy)carbonyl]amino}-4,4-difluorocyclopentane-1-carboxylate (1 g, 22.15%) as a light yellow solid.

### Step 4. Synthesis of methyl 2-[(tert-butoxycarbonyl)amino]-4,4-difluorocyclopentane-1-carboxylate

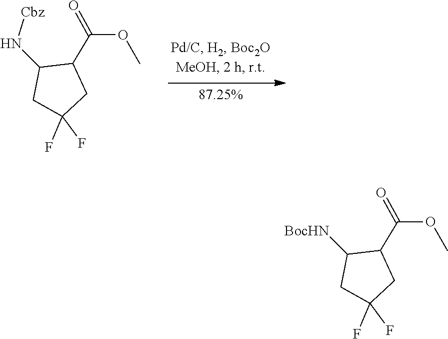 [SMILES: COC(=O)C1CC(F)(F)CC1NC(=O)OCc1ccccc1 ; COC(=O)C1CC(F)(F)CC1NC(=O)OC(C)(C)C]

To a solution of methyl 2-{[(benzyloxy)carbonyl]amino}-4,4-difluorocyclopentane-1-carboxylate (900 mg, 2.873 mmol, 1 equiv) and Boc₂O (626.95 mg, 2.873 mmol, 1 equiv) in 18 mL MeOH was added Pd/C (10%, 152.85 mg) under nitrogen atmosphere in a 50 mL 3-necked round-bottom flask. The mixture was hydrogenated at room temperature for 2 h under hydrogen atmosphere using a hydrogen balloon, filtered through a Celite pad and concentrated under reduced pressure. This resulted in methyl 2-[(tert-butoxycarbonyl)amino]-4,4-difluorocyclopentane-1-carboxylate (700 mg, 87.25%) as a white solid.

### Step 5. Synthesis of 2-[(tert-butoxycarbonyl)amino]-4,4-difluorocyclopentane-1-carboxylic acid

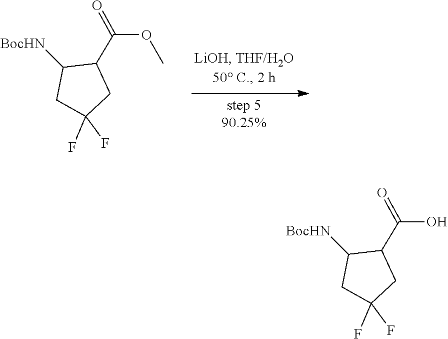 [SMILES: COC(=O)C1CC(F)(F)CC1NC(=O)OC(C)(C)C ; CC(C)(C)OC(=O)NC1CC(F)(F)CC1C(=O)O]

A mixture of methyl 2-[(tert-butoxycarbonyl)amino]-4,4-difluorocyclopentane-1-carboxylate (700 mg, 2.506 mmol, 1 equiv) and LiOH·H₂O (157.75 mg, 3.759 mmol, 1.5 equiv) in MeOH (9 mL) and H₂O (3 mL) was stirred for 4 h at 50° C. The mixture was acidified to pH=4 with HCl (aq.). The resulting mixture was extracted with CH₂Cl₂ (3×20 mL). The combined organic layers were dried over anhydrous Na₂SO₄. After filtration, the filtrate was concentrated under reduced pressure to afford 2-[(tert-butoxycarbonyl)amino]-4,4-difluorocyclopentane-1-carboxylic acid (600 mg, 90.25%) as a white solid.

### Step 6. Synthesis of 1,3-dioxoisoindol-2-yl 2-[(tert-butoxycarbonyl)amino]-4,4-difluorocyclopentane-1-carboxylate

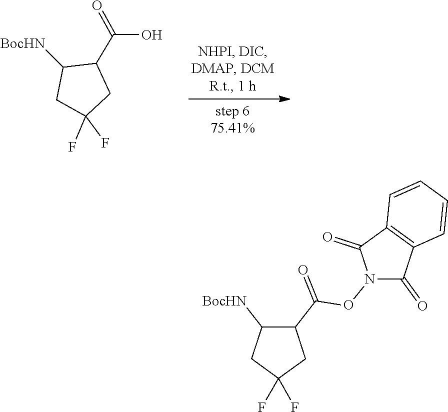 [SMILES: CC(C)(C)OC(=O)NC1CC(F)(F)CC1C(=O)O ; CC(C)(C)OC(=O)NC1CC(F)(F)CC1C(=O)ON1C(=O)c2ccccc2C1=O]

To a stirred mixture of 2-[(tert-butoxycarbonyl)amino]-4,4-difluorocyclopentane-1-carboxylic acid (600 mg, 2.262 mmol, 1 equiv), N-hydroxyphthalimide (369.00 mg, 2.262 mmol, 1 equiv) and DMAP (27.63 mg, 0.226 mmol, 0.1 equiv) in DCM (15 mL) was added DIC (314.01 mg, 2.488 mmol, 1.1 equiv) dropwise at room temperature under nitrogen atmosphere. The resulting mixture was stirred for 1 h at room temperature under nitrogen atmosphere. The resulting mixture was concentrated under reduced pressure. The residue was purified by silica gel column chromatography, eluted with PE/EA (2:1) to afford 1,3-dioxoisoindol-2-yl 2-[(tert-butoxycarbonyl)amino]-4,4-difluorocyclopentane-1-carboxylate (700 mg, 75.41%) as a white solid.

### Example 2: Specific Example of General Synthesis Scheme 2, Synthesis 5-chloro-N-(pyridin-4-ylmethyl)furo[3,2-b]pyridin-7-amine (Compound 6)

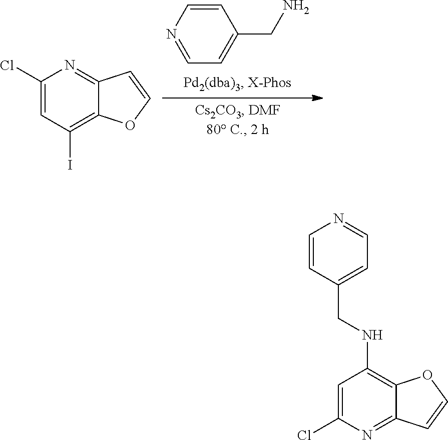 [SMILES: NCc1ccncc1 ; Clc1cc(I)c2occc2n1 ; Clc1cc(NCc2ccncc2)c2occc2n1]

Into a 25-mL vial purged and maintained with an inert atmosphere of nitrogen, was placed 5-chloro-7-iodofuro[3,2-b]pyridine (200 mg, 0.716 mmol, 1 equiv) Nemec, V. et al. Angewandte Chemie—International Edition, 2019, vol. 58, #4, p. 1062-1066) 4-pyridinemethaneamine (85.13 mg, 0.788 mmol, 1.1 equiv), Pd₂(dba)₃ (65.54 mg, 0.072 mmol, 0.1 equiv), X-Phos (68.24 mg, 0.143 mmol, 0.2 equiv), Cs₂CO₃ (466.36 mg, 1.432 mmol, 2 equiv), DMF (8 mL). The resulting solution was stirred for 2 h at 80 degrees C. The reaction was then quenched by the addition of 2 mL of water. The resulting solution was extracted with 3×10 mL of ethyl acetate and the organic layers combined and dried over anhydrous sodium sulfate. The residue was applied onto a silica gel column with dichloromethane/methanol (10:1). This resulted in 13.5 mg (7.26%) of 5-chloro-N-(pyridin-4-ylmethyl)furo[3,2-b]pyridin-7-amine.

LCMS C₁₃H₁₀ClN₃O calc. [M+H]⁺ 259.05 found 260.05.

¹H NMR (300 MHz, DMSO-d₆) δ 8.52 (d, J=5.3 Hz, 2H), 8.20 (d, J=2.2 Hz, 1H), 7.95 (q, J=6.4, 5.3 Hz, 1H), 7.39-7.29 (m, 2H), 6.93 (d, J=2.2 Hz, 1H), 6.40 (s, 1H), 4.59 (d, J=6.4 Hz, 2H).

### Example 3: Specific Example of General Synthesis Scheme 3, Synthesis of 2-[(2S)-2-aminopropyl]-5-chloro-3-cyclopropyl-N-(thiophen-2-ylmethyl)furo[3,2-b]pyridin-7-amine (Compound 19)

### Step 1. Synthesis of tert-butyl N-{5-chloro-3-cyclopropylfuro[3,2-b]pyridin-7-yl}-N-(thiophen-2-ylmethyl)carbamate

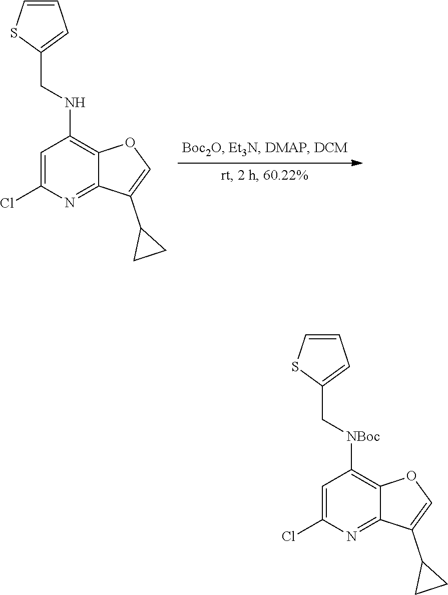 [SMILES: Clc1cc(NCc2cccs2)c2occ(C3CC3)c2n1 ; CC(C)(C)OC(=O)N(Cc1cccs1)c1cc(Cl)nc2c(C3CC3)coc12]

A solution of 5-chloro-3-cyclopropyl-N-(thiophen-2-ylmethyl)furo[3,2-b]pyridin-7-amine (500 mg, 1.640 mmol, 1 equiv) in DCM (5 mL) was treated with Boc₂O (500 mg, 2.291 mmol, 1.40 equiv) at room temperature under nitrogen atmosphere followed by the addition of DMAP (200 mg, 1.637 mmol, 1.00 equiv) in portions at room temperature. The resulting mixture was stirred for 2 h at room temperature. The resulting mixture was concentrated under reduced pressure. The residue was purified by silica gel column chromatography, eluted with PE/EA (9:1) to afford tert-butyl N-{5-chloro-3-cyclopropylfuro[3,2-b]pyridin-7-yl}-N-(thiophen-2-ylmethyl)carbamate (400 mg, 60.22%).

### Step 2. Synthesis of tert-butyl N-{2-[(2S)-2-aminopropyl]-5-chloro-3-cyclopropylfuro [3,2-b]pyridin-7-yl}-N-(thiophen-2-ylmethyl)carbamate

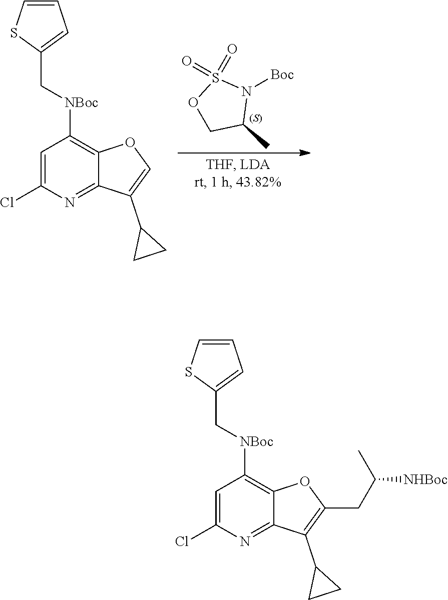

A solution of tert-butyl N-{5-chloro-3-cyclopropylfuro[3,2-b]pyridin-7-yl}-N-(thiophen-2-ylmethyl)carbamate (400 mg, 0.988 mmol, 1 equiv) in THE (10 mL) was treated with LDA (2.5 mL, 18.436 mmol, 18.66 equiv) at −78° C. under nitrogen atmosphere followed by the addition of tert-butyl (4S)-4-methyl-2,2-dioxo-1,2lambda6,3-oxathiazolidine-3-carboxylate (0.25 g, 1.057 mmol, 1.07 equiv) dropwise at −78° C. The resulting mixture was stirred for 1 h at room temperature. The reaction was quenched by the addition of Water/Ice (10 mL) at room temperature. The aqueous layer was extracted with EtOAc (3×7 mL). The residue was purified by reversed-phase flash chromatography with the following conditions: column, C₁₈; mobile phase, MeCN in Water (0.1% FA), 10% to 50% gradient in 10 min; detector, UV 254 nm. This resulted in tert-butyl N-{2-[(2S)-2-aminopropyl]-5-chloro-3-cyclopropylfuro [3,2-b]pyridin-7-yl}-N-(thiophen-2-ylmethyl)carbamate (200 mg, 43.82%) as a white solid.

### Step 3. Synthesis of 2-[(2S)-2-aminopropyl]-5-chloro-3-cyclopropyl-N-(thiophen-2-ylmethyl)furo[3,2-b]pyridin-7-amine

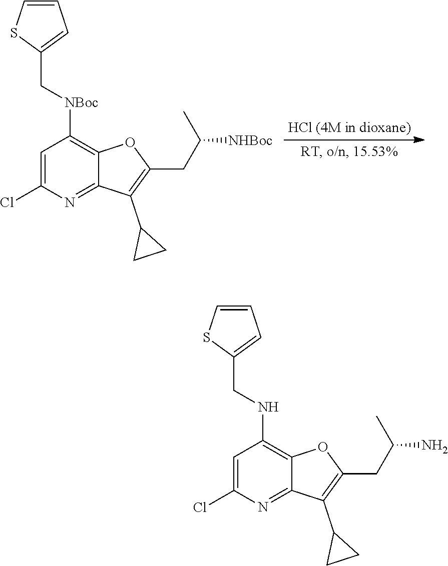 [SMILES: C[C@@H](Cc1oc2c(N(Cc3cccs3)C(=O)OC(C)(C)C)cc(Cl)nc2c1C1CC1)NC(=O)OC(C)(C)C ; C[C@H](N)Cc1oc2c(NCc3cccs3)cc(Cl)nc2c1C1CC1]

A mixture of tert-butyl N-{2-[(2S)-2-[(tert-butoxycarbonyl)amino]propyl]-5-chloro-3-cyclopropylfuro [3,2-b]pyridin-7-yl}-N-(thiophen-2-ylmethyl)carbamate (200 mg, 0.356 mmol, 1 equiv) and HCl (gas) in 1,4-dioxane (3 mL, 98.738 mmol, 277.51 equiv) was stirred for overnight at room temperature. The resulting mixture was concentrated under reduced pressure. The residue was purified by reverse flash chromatography with the following conditions: column, C₁₈; mobile phase, MeCN in Water (10 mmol/L NH₄HCO₃), 10% to 50% gradient in 10 min; detector, UV 254 nm. This resulted in 2-[(2S)-2-aminopropyl]-5-chloro-3-cyclopropyl-N-(thiophen-2-ylmethyl)furo[3,2-b]pyridin-7-amine (20 mg, 15.53%).

LC-MS [M+1]⁺=361.95

¹HNMR (300 MHz, DMSO-d₆): δ 7.66 (d, J=6.6 Hz, 3H), 7.38 (dd, J=5.1, 1.3 Hz, 3H), 7.07 (d, J=3.4 Hz, 3H), 6.97 (dd, J=5.1, 3.4 Hz, 3H), 6.77 (s, 1H), 6.44 (s, 3H), 6.05 (s, 1H), 4.67 (d, J=6.2 Hz, 6H), 3.91 (d, J=8.0 Hz, 2H), 3.24 (d, J=6.3 Hz, 1H), 3.00 (dd, J=14.6, 6.0 Hz, 1H), 2.81 (dt, J=15.0, 7.0 Hz, 3H), 1.79 (s, 4H), 1.23 (s, 1H), 1.17-0.99 (m, 7H), 0.86-0.73 (m, 6H).

### Example 4: Specific Example of General Synthesis Scheme 5: Synthesis of 2-[(S)-2-aminopropyl]-5-chloro-3-methyl-7-{[(4-pyridyl)methyl]amino}-1-oxa-4-azaindene and 2-[(R)-2-aminopropyl]-5-chloro-3-methyl-7-{[(4-pyridyl)methyl]amino}-1-oxa-4-azaindene (Compounds 3 and 4)

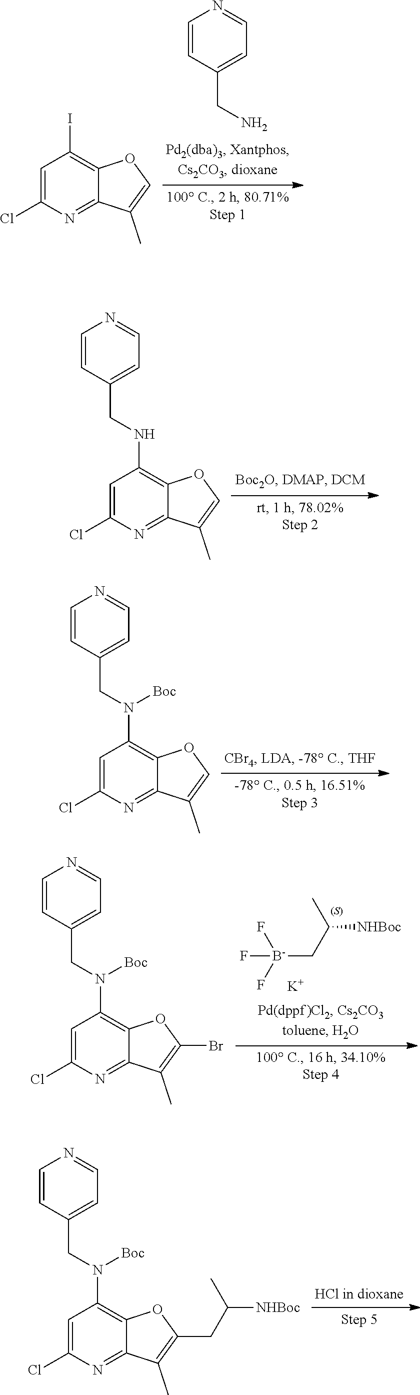

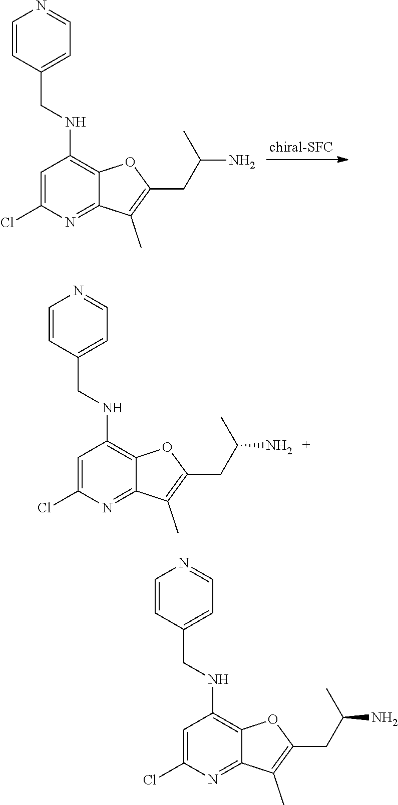

Step 1: To a solution of 5-chloro-7-iodo-3-methylfuro[3,2-b]pyridine (2 g, 6.815 mmol, 1 equiv) (Nemec, V. et al. Angewandte Chemie—International Edition, 2019, vol. 58, #4, p. 1062-1066) and benzylamine (2.19 g, 20.445 mmol, 3 equiv) in dioxane (60.00 mL) were added Pd₂(dba)₃ (624.03 mg, 0.682 mmol, 0.1 equiv), XantPhos (788.62 mg, 1.363 mmol, 0.20 equiv) and Cs₂CO₃ (4.44 g, 13.630 mmol, 2 equiv). After stirring for 2 h at 100° C. under a nitrogen atmosphere, the resulting mixture was concentrated under reduced pressure. The residue was purified by silica gel column chromatography, eluted with PE/EA (3:1) to afford 5-chloro-3-methyl-N-(pyridin-4-ylmethyl)furo[3,2-b]pyridin-7-amine (1.5 g, 80.71%) as a yellow solid.

Step 2: Into a 40 mL vial were added 5-chloro-3-methyl-N-(pyridin-4-ylmethyl)furo[3,2-b]pyridin-7-amine (1.5 g, 5.500 mmol, 1 equiv), DMAP (67.19 mg, 0.550 mmol, 0.1 equiv), DCM (30.00 mL) and Boc₂O (2.40 g, 11.000 mmol, 2 equiv) at 0° C. The resulting mixture was stirred for 1 h at room temperature. The residue was purified by silica gel column chromatography, eluted with PE/EA (5:1) to afford tert-butyl (2-bromo-5-chloro-3-methylfuro[3,2-b]pyridin-7-yl)(pyridin-4-ylmethyl)carbamate (1.6 g, 78.02%) as a yellow solid.

Step 3: In a 250-mL round bottom flask, to a solution of tert-butyl (2-bromo-5-chloro-3-methylfuro[3,2-b]pyridin-7-yl)(pyridin-4-ylmethyl)carbamate (1.5 g, 4.012 mmol, 1 equiv) in THE (70 mL) was added dropwise LDA (in 2M THF) (4.0 mL, 2 equiv) at −78° C. under N₂ atmosphere. The reaction mixture was stirred at −78° C. for 30 mins. Then a solution of CBr₄ (2.66 g, 8.024 mmol, 2 equiv) in 5 mL THE was added dropwise and the mixture was stirred for another 30 mins at −78° C. The reaction was quenched with water/sat. NH₄Cl (20 mL), and then the residue was purified by reverse flash chromatography with the following conditions: column, C₁₈; mobile phase, MeCN in Water (10 mmol/L NH₄HCO₃), 10% to 100% gradient in 20 min; detector, UV 254 nm. This resulted in tert-butyl (2-(2-((tert-butoxycarbonyl)amino)propyl)-5-chloro-3-methylfuro[3,2-b]pyridin-7-yl)(pyridin-4-ylmethyl)carbamate (300 mg, 16.51%) as a yellow solid.

Step 4: To a solution of tert-butyl (2-(2-((tert-butoxycarbonyl)amino)propyl)-5-chloro-3-methylfuro[3,2-b]pyridin-7-yl)(pyridin-4-ylmethyl)carbamate (500 mg, 1.104 mmol, 1 equiv) and tert-butyl N-[(2S)-1-[trifluoro(potassio)-lambda6-boranyl]propan-2-yl]carbamate (585.60 mg, 2.208 mmol, 2 equiv) in toluene (20.00 mL) and H₂O (2.00 mL) were added Pd(dppf)Cl₂ (80.81 mg, 0.110 mmol, 0.1 equiv) and Cs₂CO₃ (719.68 mg, 2.208 mmol, 2 equiv). After stirring for overnight at 100° C. under a nitrogen atmosphere, the resulting mixture was concentrated under reduced pressure. The residue was purified by reverse flash chromatography with the following conditions: column, C₁₈; mobile phase, MeCN in Water (10 mmol/L NH₄HCO₃), 10% to 100% gradient in 20 min; detector, UV 254 nm. This resulted in 2-(2-aminopropyl)-5-chloro-3-methyl-N-(pyridin-4-ylmethyl)furo[3,2-b]pyridin-7-amine (200 mg, 34.10%) as a yellow solid.

Step 5 and Chiral Separation: Into a 40 mL vial were added tert-butyl (2-(2-((tert-butoxycarbonyl)amino)propyl)-5-chloro-3-methylfuro[3,2-b]pyridin-7-yl)(pyridin-4-ylmethyl)carbamate (200 mg, 0.377 mmol, 1 equiv) and HCl (gas) in 1,4-dioxane (10 mL) at 0° C. The resulting mixture was stirred for 2 h at room temperature. The resulting mixture was concentrated under reduced pressure. The residue was purified by reverse flash chromatography with the following conditions: column, C₁₈; mobile phase, MeCN in Water (10 mmol/L NH₄HCO₃), 20% to 60% gradient in 10 min; detector, UV 254 nm. The crude racemic product (100 mg) was further purified by Prep-HPLC with the following conditions (Column: CHIRALPAK IE-3, 4.6*50 mm, 3 μm; Mobile Phase A: Hex (0.1% DEA):EtOH=70:30; Flow rate: 1 mL/min; Gradient: 0% B to 0% B; Injection Volume: Sul mL) to afford compound 3 (20 mg, 16.05%) as the first eluting compound and Compound 4 as the second eluting compound. The absolute configuration was not determined.

Data for Compound 3:

LC-MS (ES, m/z): [M+H]⁺=331

¹H NMR (400 MHz, Methanol-d₄) δ 8.52-8.45 (m, 2H), 7.46-7.40 (m, 2H), 6.35 (d, J=1.1 Hz, 1H), 4.66 (s, 2H), 3.39-3.30 (m, 1H), 2.87 (h, J=7.9, 7.2 Hz, 2H), 2.16 (s, 3H), 1.14 (dd, J=6.4, 1.2 Hz, 3H).

Data for Compound 4:

LC-MS (ES, m/z): [M+H]⁺=331

¹H NMR (400 MHz, Methanol-d₄) δ 8.51-8.45 (m, 2H), 7.46-7.39 (m, 2H), 6.36 (s, 1H), 4.67 (s, 2H), 3.41-3.32 (m, 1H), 2.95-2.79 (m, 2H), 2.16 (s, 3H), 1.15 (d, J=6.5 Hz, 3H).

### Example 5: Specific Example of General Method 6: Synthesis of 2-[(2R,3S)-2-amino-3-fluorobutyl]-3-bromo-5-chloro-N-(thiophen-2-ylmethyl)furo[3,2-b]pyridin-7-amine (Compound 43)

### Step 1. Synthesis of tert-butyl N-[(2R,3S)-1-[3-bromo-5-chloro-7-(methylsulfanyl)furo[3,2-b]pyridin-2-yl]-3-fluorobutan-2-yl]carbamate

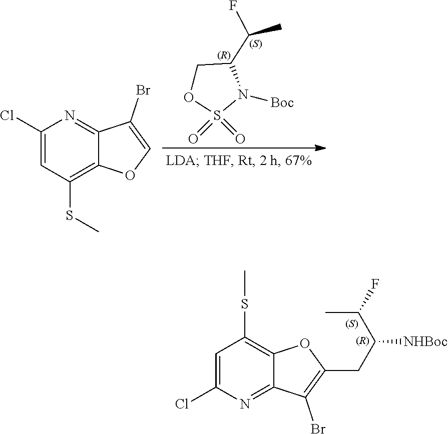

A solution of 3-bromo-5-chloro-7-(methylsulfanyl)furo[3,2-b]pyridine (400 mg, 1.436 mmol, 1 equiv) in THE (6 mL) was treated with LDA (1.08 mL, 2.154 mmol, 1.5 equiv) for 20 min at −78° C. under nitrogen atmosphere followed by the addition of tert-butyl (4R)-4-[(1S)-1-fluoroethyl]-2,2-dioxo-1,2lambda6,3-oxathiazolidine-3-carboxylate (464.04 mg, 1.723 mmol, 1.2 equiv) dropwise at −78° C. The resulting mixture was stirred for additional 2 h at room temperature. The residue was purified by reversed-phase flash chromatography with the following conditions: (column, C₁₈; mobile phase, MeCN in Water (10 mmol/L NH₄HCO₃), 10% to 100% gradient in 15 min; detector, UV 254 nm.) to afford tert-butyl N-[(2R,3S)-1-[3-bromo-5-chloro-7-(methylsulfanyl)furo[3,2-b]pyridin-2-yl]-3-fluorobutan-2-yl]carbamate (450 mg, 66.99%) as a light yellow solid.

### Step 2. Synthesis of tert-butyl N-[(2R,3S)-1-{3-bromo-5-chloro-7-methanesulfonylfuro[3,2-b]pyridin-2-yl}-3-fluorobutan-2-yl]carbamate

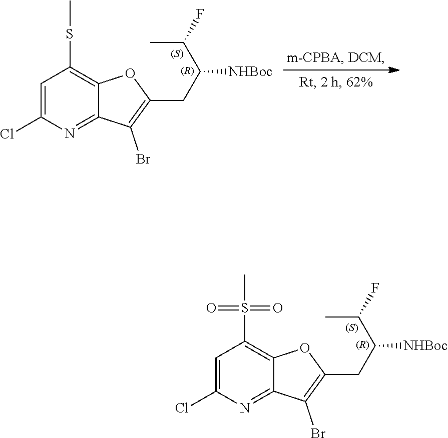

A solution of tert-butyl N-[(2R,3S)-1-[3-bromo-5-chloro-7-(methylsulfanyl)furo[3,2-b]pyridin-2-yl]-3-fluorobutan-2-yl]carbamate (450 mg, 0.962 mmol, 1 equiv) and m-CPBA (498.00 mg, 2.886 mmol, 3 equiv) in DCM (6.75 mL) was stirred for 2 h at room temperature under nitrogen atmosphere. The residue was purified by reversed-phase flash chromatography with the following conditions: (column, C₁₈; mobile phase, MeCN in Water (0.1% FA), 10% to 100% gradient in 10 min; detector, UV 254 nm.) to afford tert-butyl N-[(2R,3S)-1-{3-bromo-5-chloro-7-methanesulfonylfuro[3,2-b]pyridin-2-yl}-3-fluorobutan-2-yl]carbamate (300 mg, 62.40%) as a white solid.

### Step 3. Synthesis of 2-[(2R,3S)-2-amino-3-fluorobutyl]-3-bromo-5-chloro-N-(thiophen-2-ylmethyl)furo[3,2-b]pyridin-7-amine

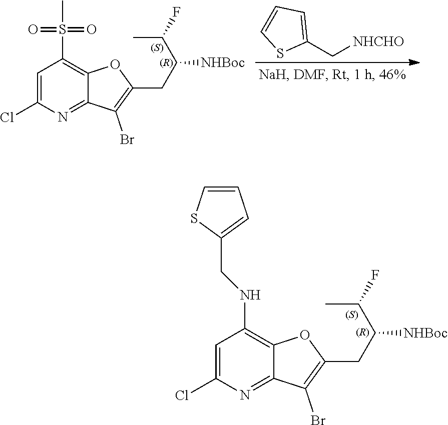

To a solution of N-(thiophen-2-ylmethyl)formamide (84.75 mg, 0.600 mmol, 1.5 equiv) in DMF (4 mL) was added sodium hydride (60% in oil, 32 mg) at 0 degrees C. The mixture was stirred for 15 min. tert-butyl N-[(2R,3S)-1-{3-bromo-5-chloro-7-methanesulfonylfuro[3,2-b]pyridin-2-yl}-3-fluorobutan-2-yl]carbamate (200 mg, 0.400 mmol, 1 equiv) was added and the mixture was allowed to warm to RT and stirred for 1 h. The reaction was quenched with Water at room temperature. The residue was purified by reversed-phase flash chromatography with the following conditions: (column, C₁₈; mobile phase, MeCN in Water (0.1% FA), 10% to 50% gradient in 10 min; detector, UV 254 nm.) to afford 2-[(2R,3S)-2-amino-3-fluorobutyl]-3-bromo-5-chloro-N-(thiophen-2-ylmethyl)furo[3,2-b]pyridin-7-amine (80 mg, 46.20%) as a light yellow solid.

### Step 4. Synthesis of 2-[(2R,3S)-2-amino-3-fluorobutyl]-3-bromo-5-chloro-N-(thiophen-2-ylmethyl)furo[3,2-b]pyridin-7-amine

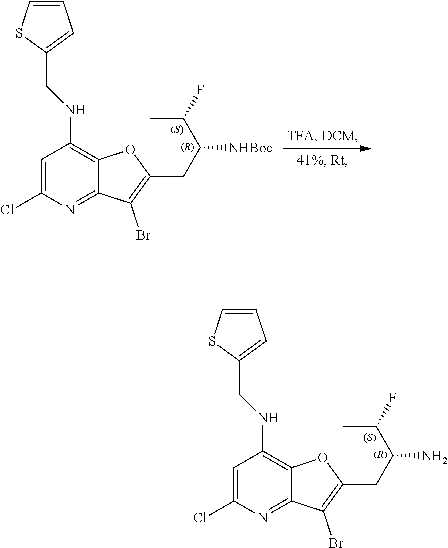

A solution of tert-butyl N-[(2R,3S)-1-{3-bromo-5-chloro-7-[(thiophen-2-ylmethyl)amino]furo[3,2-b]pyridin-2-yl}-3-fluorobutan-2-yl]carbamate (60 mg, 0.113 mmol, 1 equiv) and TFA (0.5 mL) in DCM (1.5 mL) was stirred for 1 h at room temperature under nitrogen atmosphere. The residue was purified by reversed-phase flash chromatography with the following conditions: (column, C₁₈; mobile phase, MeCN in Water (0.1% FA), 10% to 50% gradient in 10 min; detector, UV 254 nm.) to afford 2-[(2R,3S)-2-amino-3-fluorobutyl]-3-bromo-5-chloro-N-(thiophen-2-ylmethyl)furo[3,2-b]pyridin-7-amine (20 mg, 41.04%).

LC-MS\ES, m/z): [M+H]⁺=432.1.

¹H NMR (400 MHz, DMSO-d₆) δ 7.98 (t, J=6.1 Hz, 1H), 7.41 (dd, J=5.1, 1.3 Hz, 1H), 7.11 (dd, J=3.4, 1.2 Hz, 1H), 6.98 (dd, J=5.1, 3.4 Hz, 1H), 6.62 (s, 1H), 4.72 (d, J 6.1 Hz, 2H), 4.53 (dtd, J=48.3, 6.2, 2.1 Hz, 1H), 3.21 (d, J=11.3 Hz, 1H), 3.06 (dd, J=14.9, 4.4 Hz, 1H), 2.87-2.68 (m, 1H), 1.33 (dd, J=24.8, 6.2 Hz, 3H).

### Example 6: Specific Example of General Method 7, Synthesis of 2-[(2S)-2-aminopropyl]-3-methyl-7-[(thiophen-2-ylmethyl)amino]furo[3,2-b]pyridine-5-carbonitrile (Compound 15)

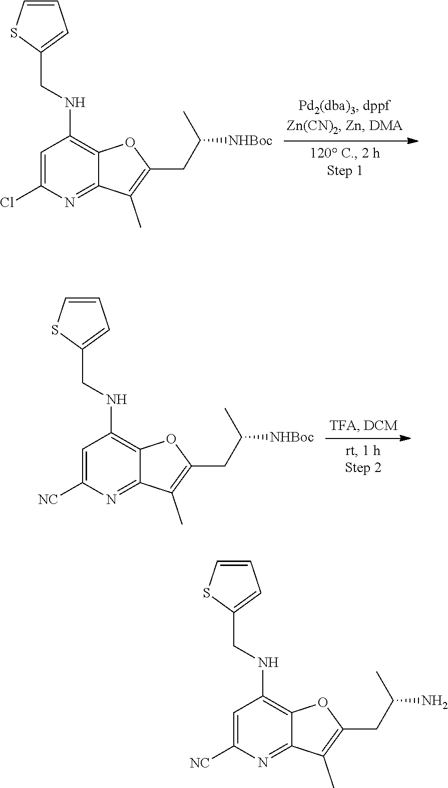 [SMILES: Cc1c(C[C@H](C)NC(=O)OC(C)(C)C)oc2c(NCc3cccs3)cc(Cl)nc12 ; Cc1c(C[C@H](C)NC(=O)OC(C)(C)C)oc2c(NCc3cccs3)cc(C#N)nc12 ; Cc1c(C[C@H](C)N)oc2c(NCc3cccs3)cc(C#N)nc12]

Step 1: To a solution of tert-butyl N-[(2S)-1-{5-chloro-3-methyl-7-[(thiophen-2-ylmethyl)amino]furo[3,2-b]-248-yridine-2-yl}propan-2-yl]carbamate (130 mg, 0.298 mmol, 1 equiv) and Zn(CN)₂ (105.04 mg, 0.894 mmol, 3 equiv) in DMA (3 mL) were added Pd₂(dba)₃ (27.31 mg, 0.030 mmol, 0.1 equiv), dppf (32.94 mg, 0.060 mmol, 0.2 equiv) and Zn (19.50 mg, 0.298 mmol, 1 equiv). After stirring for 2 h at 120° C. under a nitrogen atmosphere, the resulting mixture was concentrated under reduced pressure. The residue was purified by silica gel column chromatography, eluted with PE/EA (4:1) to afford tert-butyl N-[(2S)-1-{5-cyano-3-methyl-7-[(thiophen-2-ylmethyl)amino]furo[3,2-b]-248-yridine-2-yl}propan-2-yl]carbamate (60 mg, 47.17%) as a yellow solid.

Step 2: Into a 10 mL round-bottom flask were added tert-butyl N-{2-[(2S)-2-[(tert-butoxycarbonyl)amino]propyl]-5-cyano-3-methylfuro[3,2-b]-249-yridine-7-yl}-N-(thiophen-2-ylmethyl)carbamate (60 mg, 0.114 mmol, 1 equiv), DCM (1.5 mL) and trifluoroacetaldehyde (0.5 mL) at room temperature. The resulting mixture was stirred for 1 h at room temperature. The resulting mixture was concentrated under vacuum. The mixture was acidified to pH 8 with DIEA. The crude product was purified by Prep-HPLC with the following conditions (Column: Xbridge Prep OBD C₁₈ Column, 30*150 mm, 5 μm; Mobile Phase A: Water (10 mmol/L NH₄HCO₃), Mobile Phase B: CAN; Flow rate: 60 mL/min; Gradient: 33% B to 48% B in 7 min, 48% B; Wave Length: 254/220 nm; RT1 (min): 6.13) to afford 2-[(2S)-2-aminopropyl]-3-methyl-7-[(thiophen-2-ylmethyl)amino]furo[3,2-b]pyridine-5-carbonitrile (10 mg, 26.89%).

LC-MS (ES, m/z): [M+H]⁺=327

¹H NMR (400 MHz, DMSO-d₆) δ 7.84 (s, 1H), 7.39 (d, J=5.1 Hz, 1H), 7.12-7.07 (m, 2H), 6.97 (dt, J=4.9, 2.5 Hz, 1H), 4.75 (d, J=6.1 Hz, 2H), 3.22 (s, 1H), 2.76 (t, J=7.8 Hz, 1H), 2.11 (d, J=2.1 Hz, 2H), 1.10-0.99 (m, 3H).

### Example 7: Specific example of general method 8, synthesis of 2-[(2S)-2-aminopropyl]-3-bromo-5-chloro-N-(1,3-oxazol-2-ylmethyl) furo[3,2-b]pyridin-7-amine (Compound 124)

### Step 1. Synthesis of tert-butyl N-[(2S)-1-{3-bromo-5-chloro-7-iodofuro[3,2-b]pyridin-2-yl}propan-2-yl]carbamate

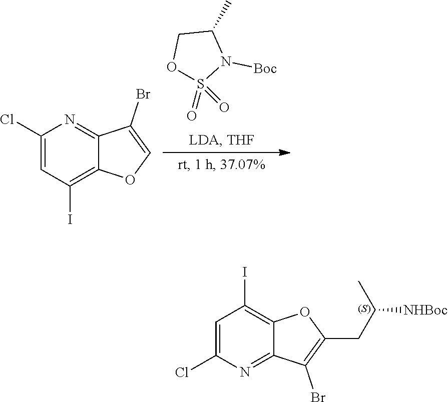

To a stirred mixture of 3-bromo-5-chloro-7-iodofuro[3,2-b]pyridine (3 g, 8.371 mmol, 1 equiv) in THE (50 mL) in portions at room temperature under nitrogen atmosphere was added LDA (5.5 mL, 40.558 mmol, 4.84 equiv) in portions at −78° C. under nitrogen atmosphere. The resulting mixture was stirred for 20 min at −78° C. under nitrogen atmosphere. To a stirred mixture of tert-butyl (4S)-4-methyl-2,2-dioxo-1,2lambda6,3-oxathiazolidine-3-carboxylate (2.6 g, 10.958 mmol, 1.31 equiv) in THF (50 mL) in portions at −78° C. under nitrogen atmosphere. The resulting mixture was stirred for 1 h at room temperature under nitrogen atmosphere. The residue was purified by reversed-phase flash chromatography with the following conditions: column, C₁₈; mobile phase, MeCN in Water (10 mmol/L NH₄HCO₃), 10% to 50% gradient in 10 min; detector, UV 254 nm. This resulted in tert-butyl N-[(2S)-1-{3-bromo-5-chloro-7-iodofuro[3,2-b]pyridin-2-yl}propan-2-yl]carbamate (1.6 g, 37.07%) as a yellow solid.

### Step 2. Synthesis of tert-butyl N-[(2S)-1-{3-bromo-5-chloro-7-[(1,3-oxazol-2-ylmethyl)amino]furo[3,2-b]pyridin-2-yl}propan-2-yl]carbamate

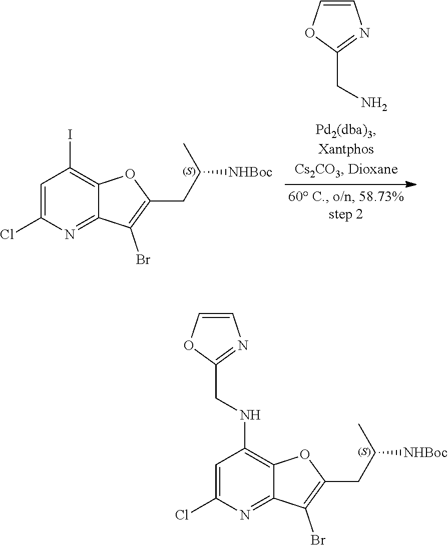

To a stirred mixture of tert-butyl N-[(2S)-1-{3-bromo-5-chloro-7-iodofuro[3,2-b]pyridin-2-yl}propan-2-yl]carbamate (150 mg, 0.291 mmol, 1 equiv) and 1-(1,3-oxazol-2-yl)methanamine (29 mg, 0.296 mmol, 1.02 equiv) in Dioxane (3 mL) were added Pd₂(dba)₃ (27 mg, 0.029 mmol, 0.10 equiv) and Xantphos (34 mg, 0.059 mmol, 0.20 equiv), caesio methaneperoxoate caesium (190 mg, 0.581 mmol, 2.00 equiv) in portions at room temperature under nitrogen atmosphere. The resulting mixture was stirred for overnight at 60° C. under nitrogen atmosphere. The residue was dissolved in DMF (1 mL). The residue was purified by reversed-phase flash chromatography with the following conditions: column, C₁₈; mobile phase, MeCN in Water (10 mmol/L NH₄HCO₃), 10% to 50% gradient in 10 min; detector, UV 254 nm. This resulted in tert-butyl N-[(2S)-1-{3-bromo-5-chloro-7-[(1,3-oxazol-2-ylmethyl)amino]furo[3,2-b]pyridin-2-yl}propan-2-yl]carbamate (83 mg, 58.73%) as a yellow solid.

### Step 3. Synthesis of 2-[(2S)-2-aminopropyl]-3-bromo-5-chloro-N-(1,3-oxazol-2-ylmethyl) furo[3,2-b]pyridin-7-amine

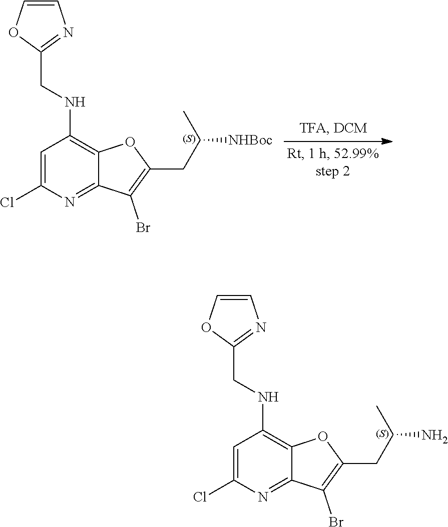

To a stirred mixture of tert-butyl N-[(2S)-1-{3-bromo-5-chloro-7-[(1,3-oxazol-2-ylmethyl) amino]furo[3,2-b]pyridin-2-yl}propan-2-yl]carbamate (82 mg, 0.169 mmol, 1 equiv) in DCM (2 mL) was added trifluoroacetaldehyde (1 mL) in portions at room temperature under nitrogen atmosphere. The resulting mixture was stirred for 1 h at room temperature under nitrogen atmosphere. The resulting mixture was concentrated under reduced pressure. The residue was dissolved in DMF (1.5 mL). The residue was purified by reversed-phase flash chromatography with the following conditions: column, C₁₈; mobile phase, MeCN in Water (0.1% TFA), 10% to 50% gradient in 10 min; detector, UV 254 nm. This resulted in 2-[(2S)-2-aminopropyl]-3-bromo-5-chloro-N-(1,3-oxazol-2-ylmethyl) furo[3,2-b]pyridin-7-amine (34.5 mg, 52.99%).

LC-MS (ES, m/z): [M+H]⁺=387.05.

¹H NMR (400 MHz, Methanol-d₄) δ 7.91 (d, J=0.9 Hz, 1H), 7.15 (d, J=0.9 Hz, 1H), 6.69 (s, 1H), 4.76 (s, 2H), 3.79 (q, J=6.6 Hz, 1H), 3.31-3.15 (m, 2H), 1.38 (d, J=6.7 Hz, 3H).

### Example 8: Specific Example of General Method 9, Synthesis of 2-[(2S)-2-aminopropyl]-5-chloro-3-ethynyl-N-(thiophen-2-ylmethyl)furo[3,2-b]pyridin-7-amine (Compound 63)

### Step 1. Synthesis of tert-butyl N-[(2S)-1-{5-chloro-7-[(thiophen-2-ylmethyl)amino]-3-[2-(trimethylsilyl)ethynyl]furo[3,2-b]pyridin-2-yl}propan-2-yl]carbamate

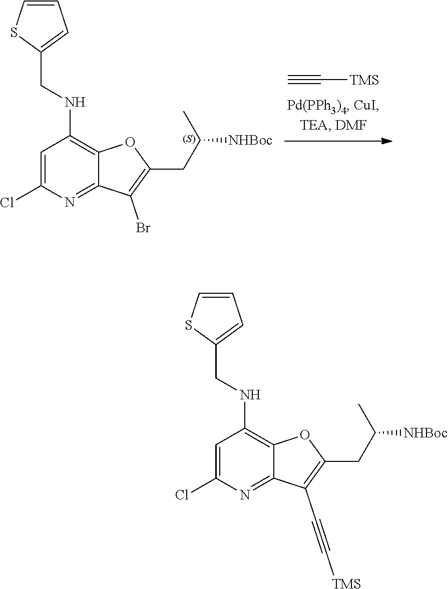

A mixture of tert-butyl N-[(2S)-1-{3-bromo-5-chloro-7-[(thiophen-2-ylmethyl)amino]furo[3,2-b]pyridin-2-yl}propan-2-yl]carbamate (500 mg, 0.998 mmol, 1 equiv), trimethylsilylacetylene (196.11 mg, 1.996 mmol, 2 equiv), Pd(PPh₃)₄ (115.37 mg, 0.100 mmol, 0.1 equiv), CuI (9.51 mg, 0.050 mmol, 0.05 equiv) and TEA (202.05 mg, 1.996 mmol, 2 equiv) in DMF (5 mL) was stirred for overnight at 80° C. under nitrogen atmosphere. The residue was purified by reversed-phase flash chromatography with the following conditions: column, C₁₈; mobile phase, MeCN in Water (10 mmol/L NH₄HCO₃), 10% to 50% gradient in 10 min; detector, UV 254 nm. This resulted in tert-butyl N-[(2S)-1-{5-chloro-7-[(thiophen-2-ylmethyl)amino]-3-[2-(trimethylsilyl)ethynyl]furo[3,2-b]pyridin-2-yl}propan-2-yl]carbamate (130 mg, 25.13%) as a yellow solid.

### Step 2. Synthesis of tert-butyl N-[(2S)-1-{5-chloro-3-ethynyl-7-[(thiophen-2-ylmethyl)amino]furo[3,2-b]pyridin-2-yl}propan-2-yl]carbamate

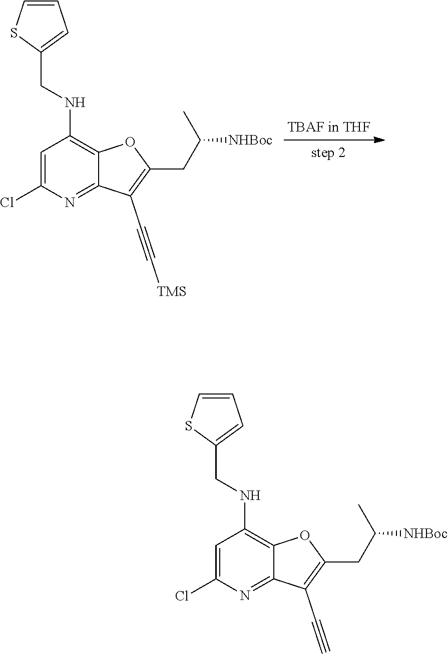 [SMILES: C[C@@H](Cc1oc2c(NCc3cccs3)cc(Cl)nc2c1C#C[Si](C)(C)C)NC(=O)OC(C)(C)C ; C#Cc1c(C[C@H](C)NC(=O)OC(C)(C)C)oc2c(NCc3cccs3)cc(Cl)nc12]

A solution of tert-butyl N-[(2S)-1-{5-chloro-7-[(thiophen-2-ylmethyl)amino]-3-[2-(trimethylsilyl)ethynyl]furo[3,2-b]pyridin-2-yl}propan-2-yl]carbamate (135 mg, 0.261 mmol, 1 equiv) in THE (5 mL) was treated with TBAF (68.12 mg, 0.261 mmol, 1 equiv) for 1 min at 0° C. under nitrogen atmosphere. The residue was purified by silica gel column chromatography, eluted with PE/EA (6:1) to afford tert-butyl N-[(2S)-1-{5-chloro-3-ethynyl-7-[(thiophen-2-ylmethyl)amino]furo[3,2-b]pyridin-2-yl}propan-2-yl]carbamate (100 mg, 86.06%) as a yellow solid.

### Step 3. Synthesis of 2-[(2S)-2-aminopropyl]-5-chloro-3-ethynyl-N-(thiophen-2-ylmethyl)furo[3,2-b]pyridin-7-amine

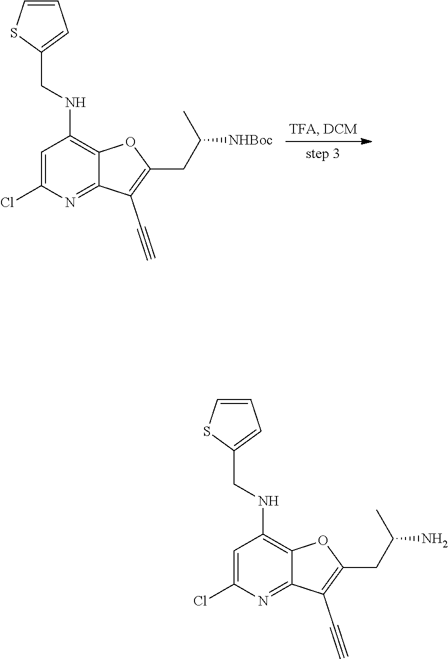 [SMILES: C#Cc1c(C[C@H](C)NC(=O)OC(C)(C)C)oc2c(NCc3cccs3)cc(Cl)nc12 ; C#Cc1c(C[C@H](C)N)oc2c(NCc3cccs3)cc(Cl)nc12]

A solution of tert-butyl N-[(2S)-1-{5-chloro-3-ethynyl-7-[(thiophen-2-ylmethyl)amino]furo[3,2-b]pyridin-2-yl}propan-2-yl]carbamate (100 mg, 0.224 mmol, 1 equiv) in DCM (4 mL) was treated with TFA (2 mL) for 1 min at room temperature. The reaction was monitored by LCMS. The crude product (90 mg) was purified by Prep-HPLC with the following conditions (Column: XBridge Prep C18 OBD Column, 19*150 mm, 5 μm; Mobile Phase A: Water (10 mmol/L NH₄HCO₃), Mobile Phase B: ACN; Flow rate: 25 mL/min; Gradient: 10% B to 35% B in 7 min, 35% B; Wave Length: 254/220 nm; RT1 (min): 6.35) to afford 2-[(2S)-2-aminopropyl]-5-chloro-3-ethynyl-N-(thiophen-2-ylmethyl)furo[3,2-b]pyridin-7-amine (23 mg, 29.66%).

LCMS: [M+H]⁺=346.

¹H NMR (400 MHz, Methanol-d₄) δ 7.30 (dd, J=5.1, 1.2 Hz, 1H), 7.08 (dd, J=3.5, 1.2 Hz, 1H), 6.97 (dd, J=5.1, 3.5 Hz, 1H), 6.58 (s, 1H), 4.75 (s, 2H), 3.91 (s, 1H), 3.60 (h, J=6.5 Hz, 1H), 3.20-3.02 (m, 2H), 1.25 (d, J=6.5 Hz, 3H).

### Example 9: Specific Example of General Method 10, Synthesis of 2-[(R)-2-amino-3-pentynyl]-3-bromo-5-chloro-7-thenylamino-1-oxa-4-azaindene (Compound 132)

### Step 1. Synthesis of tert-butyl N-[(2R)-1-{3-bromo-5-chloro-7-iodofuro[3,2-b]pyridin-2-yl}-3-[(tert-butyldimethylsilyl)oxy]propan-2-yl]carbamate

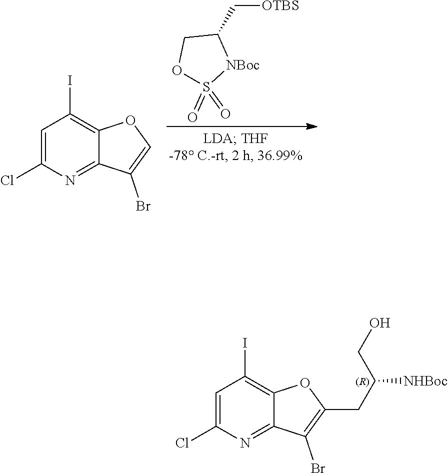

A solution of 3-bromo-5-chloro-7-iodofuro[3,2-b]pyridine (3 g, 8.371 mmol, 1 equiv) in THE (40 mL) was treated with LDA (5.44 mL, 10.882 mmol, 1.3 equiv) for 20 min at −78° C. under nitrogen atmosphere followed by the addition of tert-butyl (4S)-4-{[(tert-butyldimethylsilyl)oxy]methyl}-2,2-dioxo-1,2lambda6,3-oxathiazolidine-3-carboxylate (3.69 g, 10.045 mmol, 1.2 equiv) dropwise at −78° C. The resulting mixture was stirred for additional 2 h at room temperature. The residue was purified by reversed-phase flash chromatography with the following conditions: (column, C₁₈; mobile phase, MeCN in Water (10 mmol/L NH₄HCO₃), 10% to 100% gradient in 10 min; detector, UV 254 nm.) to afford tert-butyl N-[(2R)-1-{3-bromo-5-chloro-7-iodofuro[3,2-b]pyridin-2-yl}-3-[(tert-butyldimethylsilyl)oxy]propan-2-yl]carbamate (2 g, 36.99%) as a light yellow solid.

### Step 2: Synthesis of tert-butyl N-[(2R)-1-{3-bromo-5-chloro-7-iodofuro[3,2-b]pyridin-2-yl}-3-oxopropan-2-yl]carbamate

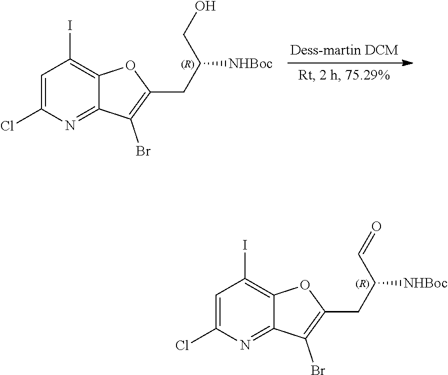

A solution of tert-butyl N-[(2R)-1-{3-bromo-5-chloro-7-iodofuro[3,2-b]pyridin-2-yl}-3-hydroxypropan-2-yl]carbamate (400 mg, 0.752 mmol, 1 equiv) and Dess-martin (382.99 mg, 0.902 mmol, 1.2 equiv) in DCM (7 mL) was stirred for 2 h at room temperature under nitrogen atmosphere. The resulting mixture was extracted with CH₂Cl₂ (1×2 20 mL). The combined organic layers were washed with DCM (1×1 20 mL), dried over anhydrous Na₂SO₄. After filtration, the filtrate was concentrated under reduced pressure to afford tert-butyl N-[(2R)-1-{3-bromo-5-chloro-7-iodofuro[3,2-b]pyridin-2-yl}-3-oxopropan-2-yl]carbamate (300 mg, 75.29%) as a light yellow solid.

### Step 3. Synthesis of tert-butyl N-[(2R)-1-{3-bromo-5-chloro-7-iodofuro[3,2-b]pyridin-2-yl}but-3-yn-2-yl]carbamate

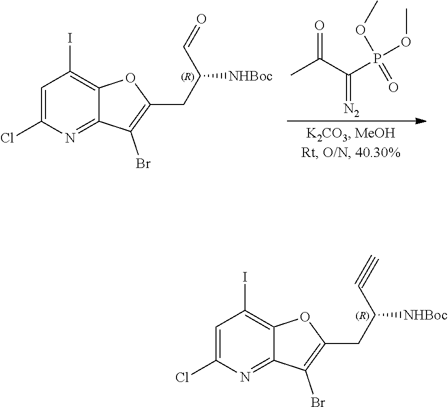

To a stirred solution of tert-butyl N-[(2R)-1-{3-bromo-5-chloro-7-iodofuro[3,2-b]pyridin-2-yl}-3-oxopropan-2-yl]carbamate (300 mg, 0.567 mmol, 1 equiv) and potassium methaneperoxoate potassium (157.73 mg, 1.134 mmol, 2 equiv) in methanol (4.5 mL) was added seyferth-gilbert homologation (141.48 mg, 0.737 mmol, 1.3 equiv) dropwise at 0° C. under nitrogen atmosphere. The resulting mixture was stirred for additional overnight at room temperature. The residue was purified by reversed-phase flash chromatography with the following conditions: (column, C₁₈; mobile phase, MeCN in Water (0.1% FA), 10% to 50% gradient in 10 min; detector, UV 254 nm.) to afford tert-butyl N-[(2R)-1-{3-bromo-5-chloro-7-iodofuro[3,2-b]pyridin-2-yl}but-3-yn-2-yl]carbamate (120 mg, 40.30%) as a light yellow solid.

### Step 4. Synthesis of tert-butyl N-[(2R)-1-{3-bromo-5-chloro-7-iodofuro[3,2-b]pyridin-2-yl}pent-3-yn-2-yl]carbamate

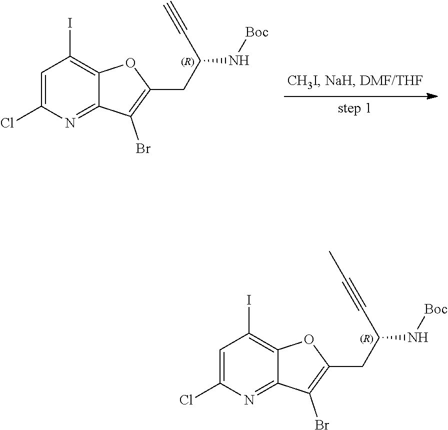

A solution of tert-butyl N-[(2R)-1-{3-bromo-5-chloro-7-iodofuro[3,2-b]pyridin-2-yl}but-3-yn-2-yl]carbamate (50 mg, 0.095 mmol, 1 equiv) in DMF/THF (1/3, 4 mL) was treated with NaH (2.97 mg, 0.124 mmol, 1.3 equiv, 60%) for 30 min at 0° C. under nitrogen atmosphere. To the above mixture was added CH₃I (14.85 mg, 0.105 mmol, 1.1 equiv) dropwise over 1 min at 0° C. The resulting mixture was stirred for additional overnight at room temperature. The residue was purified by silica gel column chromatography, eluted with PE/EA (6:1) to afford tert-butyl N-[(2R)-1-{3-bromo-5-chloro-7-iodofuro[3,2-b]pyridin-2-yl}pent-3-yn-2-yl]carbamate as a yellow solid.

### Step 5. Synthesis of tert-butyl N-[(2R)-1-{3-bromo-5-chloro-7-[(thiophen-2-ylmethyl)amino]furo[3,2-b]pyridin-2-yl}pent-3-yn-2-yl]carbamate

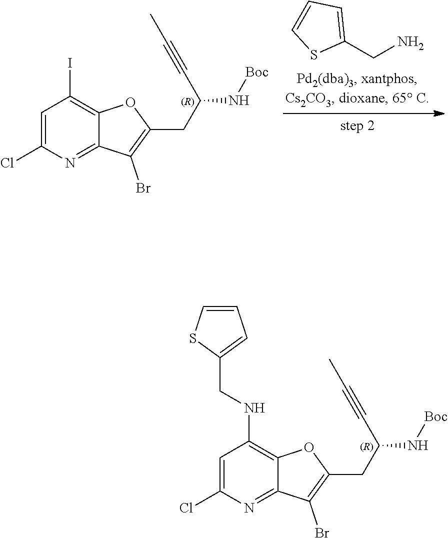

A solution of tert-butyl N-[(2R)-1-{3-bromo-5-chloro-7-iodofuro[3,2-b]pyridin-2-yl}pent-3-yn-2-yl]carbamate (60 mg, 0.111 mmol, 1 equiv), 1-(thiophen-2-yl)methanamine (25.17 mg, 0.222 mmol, 2 equiv), Pd₂(dba)₃ (10.18 mg, 0.011 mmol, 0.1 equiv), xantphos (12.87 mg, 0.022 mmol, 0.2 equiv) and Cs₂CO₃ (72.46 mg, 0.222 mmol, 2 equiv) in dioxane (6 mL) was stirred for 3 h at 65° C. under nitrogen atmosphere. The residue was purified by silica gel column chromatography, eluted with PE/EA (1:2) to afford tert-butyl N-[(2R)-1-{3-bromo-5-chloro-7-[(thiophen-2-ylmethyl)amino]furo[3,2-b]pyridin-2-yl}pent-3-yn-2-yl]carbamate (30 mg, 51.40%) as a yellow solid.

### Step 6. 2-[(2R)-2-aminopent-3-yn-1-yl]-3-bromo-5-chloro-N-(thiophen-2-ylmethyl)furo[3,2-b]pyridin-7-amine

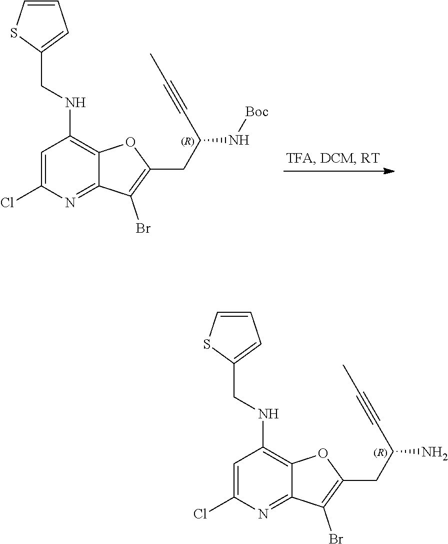

A solution of tert-butyl N-[(2R)-1-{3-bromo-5-chloro-7-[(thiophen-2-ylmethyl)amino]furo[3,2-b]pyridin-2-yl}pent-3-yn-2-yl]carbamate (25 mg, 0.048 mmol, 1 equiv) in DCM (2 mL) was treated with TFA (1 mL) for 1 min at room temperature. The reaction was monitored by LCMS. The crude product (25 mg) was purified by Prep-HPLC with the following conditions (Column: XBridge Prep C18 OBD Column, 19*150 mm, 5 μm; Mobile Phase A: Water (10 mmol/L NH₄HCO₃), Mobile Phase B: ACN; Flow rate: 25 mL/min; Gradient: 10% B to 35% B in 7 min, 35% B; Wave Length: 254/220 nm; RT1 (min): 6.35) to afford 2-[(2R)-2-aminopent-3-yn-1-yl]-3-bromo-5-chloro-N-(thiophen-2-ylmethyl)furo[3,2-b]-256-yridine-7-amine (4.2 mg, 20.76%).

LCMS [M+H]⁺=426.

¹H NMR (400 MHz, Methanol-d₄) δ 7.31 (dd, J=5.1, 1.2 Hz, 1H), 7.08 (dt, J=3.5, 1.1 Hz, 1H), 6.98 (dd, J=5.1, 3.5 Hz, 1H), 6.61 (s, 1H), 4.80-4.71 (m, 2H), 4.11 (td, J=7.1, 2.2 Hz, 1H), 2.94 (d, J=2.2 Hz, 1H), 2.60 (s, 3H).

### Example 10: Specific Example of General Method 11, Synthesis of 2-[(S)-2-amino-1,1-difluoropropyl]-3-bromo-5-chloro-7-thenylamino-1-oxa-4-azaindene (Compound 187)

### Step 1. Synthesis of tert-butyl N-[(2S)-1-{3-bromo-5-chloro-7-iodofuro[3,2-b]pyridin-2-yl}-1-hydroxypropan-2-yl]carbamate

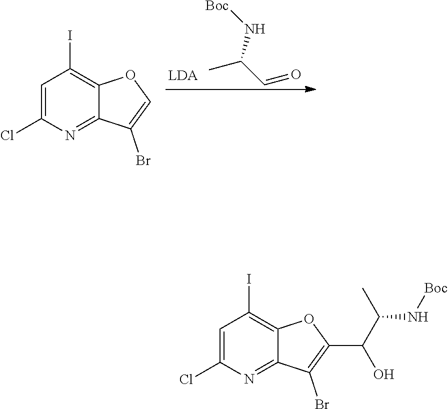 [SMILES: Clc1cc(I)c2occ(Br)c2n1 ; C[C@@H](C=O)NC(=O)OC(C)(C)C ; C[C@H](NC(=O)OC(C)(C)C)C(O)c1oc2c(I)cc(Cl)nc2c1Br]

To a stirred solution 3-bromo-5-chloro-7-iodofuro[3,2-b]pyridine (1.2 g, 3.349 mmol, 1 equiv) in THE (10 mL) was added LDA (2.8 mL, 20.648 mmol, 6.17 equiv) dropwise −78° C. under nitrogen atmosphere. The resulting mixture was stirred for additional 40 min at −78° C. To the above mixture was added tert-butyl N-[(2S)-1-oxopropan-2-yl]carbamate (0.87 g, 5.024 mmol, 1.5 equiv) in THE (10 mL) dropwise 5 min at −78° C. The resulting mixture was stirred for additional 3 h at 0° C. The reaction was quenched by the addition of Water (0.5 mL) at room temperature. The residue was purified by reversed-phase flash chromatography with the following conditions: column, C₁₈; mobile phase, MeCN in Water (0.1% FA), 10% to 50% gradient in 10 min; detector, UV 254 nm. This resulted in tert-butyl N-[(2S)-1-{3-bromo-5-chloro-7-iodofuro[3,2-b]pyridin-2-yl}-1-hydroxypropan-2-yl]carbamate (385 mg, 21.63%) as a light yellow solid.

### Step 2. Synthesis of tert-butyl N-[(2S)-1-{3-bromo-5-chloro-7-iodofuro[3,2-b]pyridin-2-yl}-1-oxopropan-2-yl]carbamate

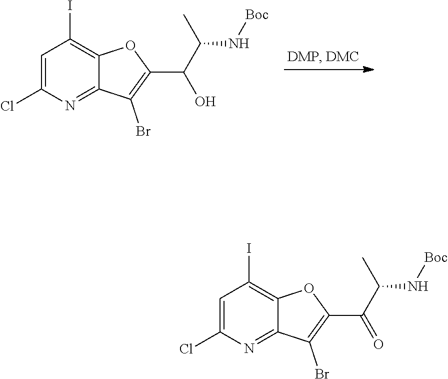 [SMILES: C[C@H](NC(=O)OC(C)(C)C)C(O)c1oc2c(I)cc(Cl)nc2c1Br ; C[C@H](NC(=O)OC(C)(C)C)C(=O)c1oc2c(I)cc(Cl)nc2c1Br]

To a stirred solution tert-butyl N-[(2S)-1-{3-bromo-5-chloro-7-iodofuro[3,2-b]pyridin-2-yl}-1-hydroxypropan-2-yl]carbamate (370 mg, 0.696 mmol, 1 equiv) in DCM (5 mL) was added Dess-Martin (442.84 mg, 1.044 mmol, 1.5 equiv) in portions at 0° C. under air atmosphere. The resulting mixture was stirred for additional 5 h at room temperature. The reaction was quenched by the addition of 1M Na₂SO₃ (3 mL) at 0° C. The mixture was basified to pH 8 with saturated NaHCO₃ (aq.). The resulting mixture was extracted with CH₂Cl₂ (3×50 mL). The combined organic layers were washed with brine (1×7 mL), dried over anhydrous Na₂SO₄. After filtration, the filtrate was concentrated under reduced pressure. The residue was purified by silica gel column chromatography, eluted with PE/EA (1:1) to afford tert-butyl N-[(2S)-1-{3-bromo-5-chloro-7-iodofuro[3,2-b]pyridin-2-yl}-1-oxopropan-2-yl]carbamate (360 mg, 97.67%) as a white solid.

### Step 3. Synthesis of tert-butyl N-[(2S)-1-{3-bromo-5-chloro-7-iodofuro[3,2-b]pyridin-2-yl}-1,1-difluoropropan-2-yl]carbamate

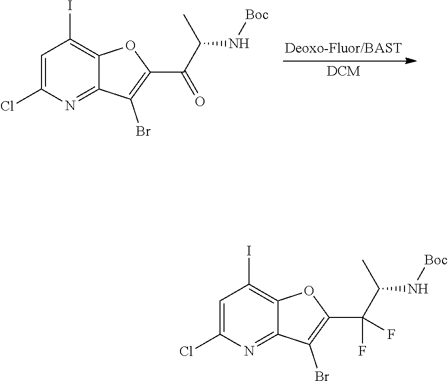 [SMILES: C[C@H](NC(=O)OC(C)(C)C)C(=O)c1oc2c(I)cc(Cl)nc2c1Br ; C[C@H](NC(=O)OC(C)(C)C)C(F)(F)c1oc2c(I)cc(Cl)nc2c1Br]

A solution tert-butyl N-[(2S)-1-{3-bromo-5-chloro-7-iodofuro[3,2-b]pyridin-2-yl}-1-oxopropan-2-yl]carbamate (330 mg, 0.623 mmol, 1 equiv) in DAST (13.2 mL) was stirred for overnight at room temperature under air atmosphere. To the above mixture was added BAST (620.42 mg, 2.804 mmol, 4.5 equiv) in portions over 0.5 min at 0° C. The resulting mixture was stirred for additional 8 h at room temperature. The reaction was quenched with 50 mL sat. NaHCO₃ (aq.) at 0° C. The resulting mixture was extracted with EtOAc (3×50 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (4:1) to afford tert-butyl N-[(2S)-1-{3-bromo-5-chloro-7-iodofuro[3,2-b]pyridin-2-yl}-1,1-difluoropropan-2-yl]carbamate (77 mL, 22.40%) as a light yellow solid.

### Step 4. Synthesis of tert-butyl N-[(2S)-1-{3-bromo-5-chloro-7-[(1,3-thiazol-2-ylmethyl)amino]furo[3,2-b]pyridin-2-yl}-1,1-difluoropropan-2-yl]carbamate

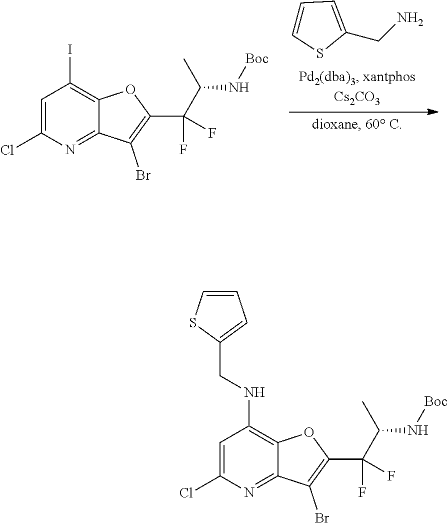 [SMILES: C[C@H](NC(=O)OC(C)(C)C)C(F)(F)c1oc2c(I)cc(Cl)nc2c1Br ; NCc1cccs1 ; C[C@H](NC(=O)OC(C)(C)C)C(F)(F)c1oc2c(NCc3cccs3)cc(Cl)nc2c1Br]

A mixture of tert-butyl N-[(2S)-1-{3-bromo-5-chloro-7-iodofuro[3,2-b]pyridin-2-yl}-1,1-difluoropropan-2-yl]carbamate (48 mg, 0.087 mmol, 1 equiv), 1-(1,3-thiazol-2-yl)methanamine (9.94 mg, 0.087 mmol, 1 equiv), Pd₂(dba)₃ (3.98 mg, 0.004 mmol, 0.05 equiv), XantPhos (5.04 mg, 0.009 mmol, 0.1 equiv) and Cs₂CO₃ (56.71 mg, 0.174 mmol, 2 equiv) in 1,4-dioxane (2 mL) was stirred for overnight at 0° C. under a nitrogen atmosphere. The resulting mixture was concentrated under reduced pressure. The residue was purified by silica gel column chromatography, eluted with PE/EA (4:1) to afford tert-butyl N-[(2S)-1-{3-bromo-5-chloro-7-[(1,3-thiazol-2-ylmethyl)amino]furo[3,2-b]pyridin-2-yl}-1,1-difluoropropan-2-yl]carbamate (40 mg, 85.46%) as a light yellow solid.

### Step 5. Synthesis of 2-[(2S)-2-amino-1,1-difluoropropyl]-3-bromo-5-chloro-N-(thiophen-2-ylmethyl)furo[3,2-b]pyridin-7-amine

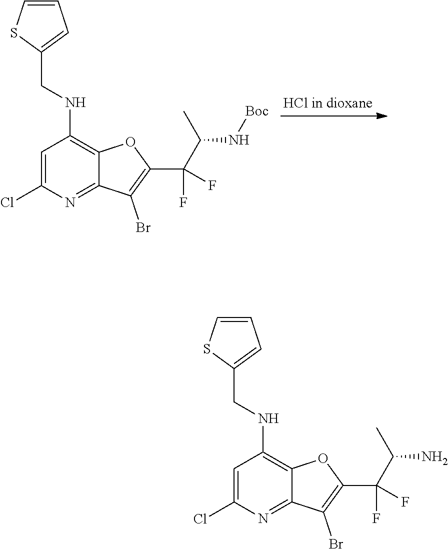 [SMILES: C[C@H](NC(=O)OC(C)(C)C)C(F)(F)c1oc2c(NCc3cccs3)cc(Cl)nc2c1Br ; C[C@H](N)C(F)(F)c1oc2c(NCc3cccs3)cc(Cl)nc2c1Br]

To a stirred solution tert-butyl N-[(2S)-1-{3-bromo-5-chloro-7-[(thiophen-2-ylmethyl)amino]furo[3,2-b]pyridin-2-yl}-1,1-difluoropropan-2-yl]carbamate (60 mg, 0.112 mmol, 1 equiv) in DCM (1 mL) was added HCl(gas) in 1,4-dioxane (1 mL, 4M) dropwise 0° C. under air atmosphere. The resulting mixture was stirred for additional 2 h at room temperature. The resulting mixture was concentrated under reduced pressure. The residue was dissolved in MeOH (1 mL). The crude product was purified by Prep-HPLC with the following conditions (Column: XBridge Prep C18 OBD Column 30*100 mm, 5 m 13 nm; Mobile Phase A: Water (10 mmol/L NH₄HCO₃+0.05% NH₃·H₂O), Mobile Phase B: ACN; Flow rate: 50 mL/min mL/min; Gradient: 25% B to 65% B in 10 min; Wave Length: 254 nm/220 nm nm; RT1 (min): 6.05) to afford 2-[(2S)-2-amino-1,1-difluoropropyl]-3-bromo-5-chloro-N-(thiophen-2-ylmethyl)furo[3,2-b]pyridin-7-amine.

LC-MS (ES, m/z): [M+H]⁺=436

¹H NMR (400 MHz, Methanol-d₄) δ 7.31 (dd, J=5.1, 1.2 Hz, 1H), 7.09 (dt, J=3.3, 1.1 Hz, 1H), 6.98 (dd, J=5.1, 3.5 Hz, 1H), 6.69 (s, 1H), 4.77 (s, 2H), 3.65 (dt, J=12.3, 6.1 Hz, 1H), 1.21 (d, J=6.8 Hz, 3H).

### Example 11: Specific Example of General Synthesis Scheme 12, Synthesis of (S)-2-(2-amino-4-(difluoromethoxy)butyl)-3-bromo-5-chloro-N-(thiophen-2-ylmethyl)furo[3,2-b]pyridin-7-amine (Compound 154)

### Step 1. Synthesis of tert-butyl N-[(2S)-1-{3-bromo-5-chloro-7-iodofuro[3,2-b]pyridin-2-yl}-4-hydroxybutan-2-yl]carbamate

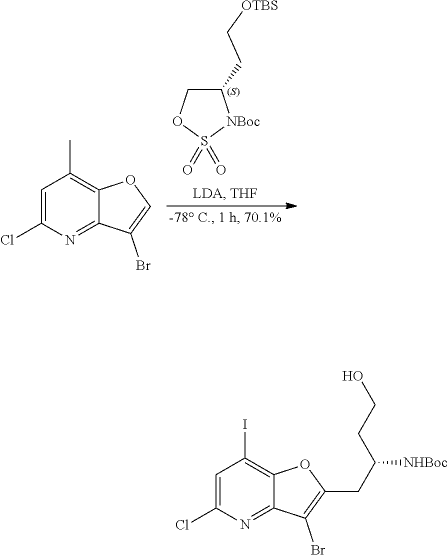

Into a 50-mL three-necked bottle, to a solution of 3-bromo-5-chloro-7-iodofuro[3,2-b]pyridine (2.5 g, 6.976 mmol, 1 equiv) in THE (20 mL) was added LDA (5.23 mL, 10.464 mmol, 1.5 equiv) dropwise at −78 degrees C. under N₂ atmosphere. The reaction mixture was stirred at −78 degrees C. for 30 mins. Then a solution of tert-butyl (4S)-4-{2-[(tert-butyldimethylsilyl)oxy]ethyl}-2,2-dioxo-1,2lambda6,3-oxathiazolidine-3-carboxylate (3.99 g, 10.464 mmol, 1.5 equiv) in 10 mL THE was added dropwise and the mixture was stirred for another 1 h −78 degrees C. The residue was purified by reversed-phase flash chromatography with the following conditions: column, C₁₈; mobile phase, MeCN in Water (10 mmol/L NH₄HCO₃), 20% to 100% gradient in 20 min; detector, UV 254 nm. This resulted in tert-butyl N-[(2S)-1-{3-bromo-5-chloro-7-iodofuro[3,2-b]pyridin-2-yl}-4-hydroxybutan-2-yl]carbamate (2.67 g, 70.15%) as a yellow solid.

### Step 2. Synthesis of tert-butyl N-[(2S)-1-{3-bromo-5-chloro-7-iodofuro[3,2-b]pyridin-2-yl}-4-(difluoromethoxy)butan-2-yl]carbamate

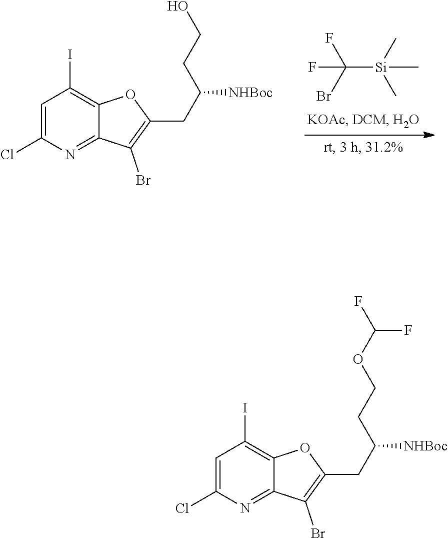

To a stirred mixture of tert-butyl N-[(2S)-1-{3-bromo-5-chloro-7-iodofuro[3,2-b]pyridin-2-yl}-4-hydroxybutan-2-yl]carbamate (410 mg, 0.751 mmol, 1 equiv) and (bromodifluoromethyl)trimethylsilane (457.87 mg, 2.253 mmol, 3 equiv) in DCM (4 mL) and H₂O (4 mL) was added KOAc (442.50 mg, 4.506 mmol, 6 equiv). The resulting mixture was stirred for 3 h at room temperature. The resulting mixture was extracted with DCM (3×5 mL). The combined organic layers were dried over anhydrous Na₂SO₄. After filtration, the filtrate was concentrated under reduced pressure. The residue was purified by reversed-phase flash chromatography with the following conditions: column, C₁₈; mobile phase, MeCN in Water (10 mmol/L NH₄HCO₃), 20% to 100% gradient in 20 min; detector, UV 254 nm. This resulted in tert-butyl N-[(2S)-1-{3-bromo-5-chloro-7-iodofuro[3,2-b]pyridin-2-yl}-4-(difluoromethoxy)butan-2-yl]carbamate (140 mg, 31.28%) as a white solid.

### Step 3. Synthesis tert-butyl N-[(2S)-1-{3-bromo-5-chloro-7-[(thiophen-2-ylmethyl)amino]furo[3,2-b]pyridin-2-yl}-4-(difluoromethoxy)butan-2-yl]carbamate

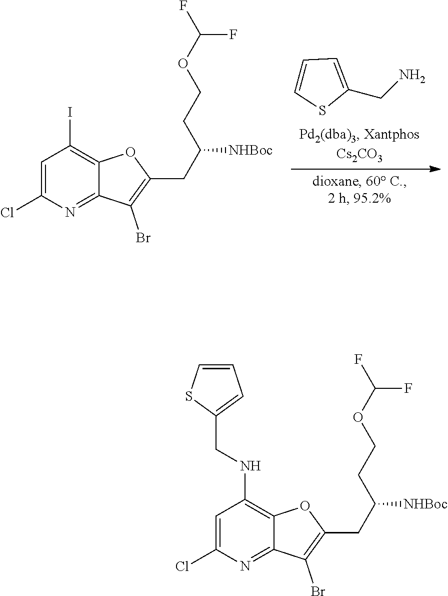 [SMILES: CC(C)(C)OC(=O)N[C@@H](CCOC(F)F)Cc1oc2c(I)cc(Cl)nc2c1Br ; NCc1cccs1 ; CC(C)(C)OC(=O)N[C@@H](CCOC(F)F)Cc1oc2c(NCc3cccs3)cc(Cl)nc2c1Br]

Into a 8-mL vial purged and maintained with an inert atmosphere of nitrogen, were added tert-butyl N-[(2S)-1-{3-bromo-5-chloro-7-iodofuro[3,2-b]pyridin-2-yl}-4-(difluoromethoxy)butan-2-yl]carbamate (140 mg, 0.235 mmol, 1 equiv), 1-(thiophen-2-yl)methanamine (24.12 uL, 0.235 mmol, 1 equiv), Pd₂(dba)₃ (21.52 mg, 0.024 mmol, 0.1 equiv), Xantphos (27.20 mg, 0.047 mmol, 0.2 equiv) and Cs₂CO₃ (153.17 mg, 0.470 mmol, 2 equiv) in Dioxane (2 mL) at room temperature. The resulting mixture was stirred for 2 h at 60° C. under nitrogen atmosphere. The residue was purified by reversed-phase flash chromatography with the following conditions: column, C₁₈; mobile phase, MeCN in Water (10 mmol/L NH₄HCO₃), 10% to 100% gradient in 20 min; detector, UV 254 nm. This resulted in tert-butyl N-[(2S)-1-{3-bromo-5-chloro-7-[(thiophen-2-ylmethyl)amino]furo[3,2-b]pyridin-2-yl}-4-(difluoromethoxy)butan-2-yl]carbamate (130 mg, 95.21%) as a yellow solid.

### Step 4. Synthesis of (S)-2-(2-amino-4-(difluoromethoxy)butyl)-3-bromo-5-chloro-N-(thiophen-2-ylmethyl)furo[3,2-b]pyridin-7-amine

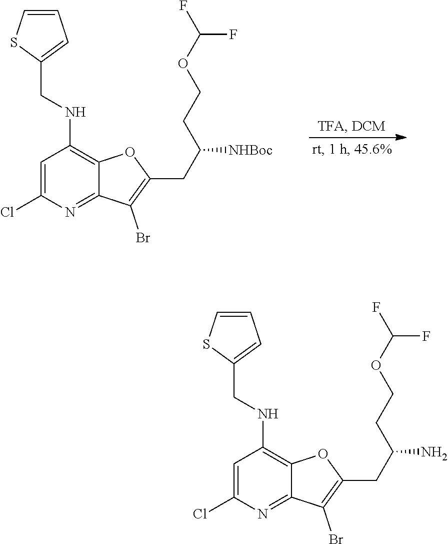 [SMILES: CC(C)(C)OC(=O)N[C@@H](CCOC(F)F)Cc1oc2c(NCc3cccs3)cc(Cl)nc2c1Br ; N[C@@H](CCOC(F)F)Cc1oc2c(NCc3cccs3)cc(Cl)nc2c1Br]

To a stirred solution of tert-butyl N-[(2S)-1-{3-bromo-5-chloro-7-[(thiophen-2-ylmethyl)amino]furo[3,2-b]pyridin-2-yl}-4-(difluoromethoxy)butan-2-yl]carbamate (130 mg, 0.224 mmol, 1 equiv) in DCM (1 mL) was added TFA (0.3 mL) dropwise at 0° C. The resulting mixture was stirred for 1 h at room temperature. The resulting mixture was concentrated under vacuum. The residue was purified by reversed-phase flash chromatography with the following conditions: column, C₁₈; mobile phase, MeCN in Water (10 mmol/L NH₄HCO₃), 0% to 100% gradient in 20 min; detector, UV 254 nm. This resulted in (S)-2-(2-amino-4-(difluoromethoxy)butyl)-3-bromo-5-chloro-N-(thiophen-2-ylmethyl)furo[3,2-b]pyridin-7-amine (78.1 mg, 45.69%).

LC-MS (ES, m/z): [M+H]⁺=480

(300 MHz, Methanol-d₄) δ 8.81 (s, 1H), 7.29-7.19 (m, 1H), 7.04-6.93 (m, 2H), 3.46-3.35 (m, 1H), 3.20-3.01 (m, 2H), 2.88-2.76 (m, 1H), 2.59 (s, 3H), 2.47-2.37 (m, 1H), 2.03-1.93 (m, 1H), 1.79-1.69 (m, 1H), 1.23 (d, J=6.4 Hz, 3H).

### Example 12: Specific Example of General Method 13, Synthesis of 2-[(1R,2R)-2-aminocyclobutyl]-3-bromo-5-chloro-N-(thiophen-2-ylmethyl)furo[3,2-b]pyridin-7-amine and 2-[(1S,2S)-2-aminocyclobutyl]-3-bromo-5-chloro-N-(thiophen-2-ylmethyl)furo[3,2-b]pyridin-7-amine (Compounds 367, 290, and 291)

### Step 1. Synthesis of 1,3-dioxoisoindol-2-yl 2-[(tert-butoxycarbonyl)amino]cyclobutane-1-carboxylate

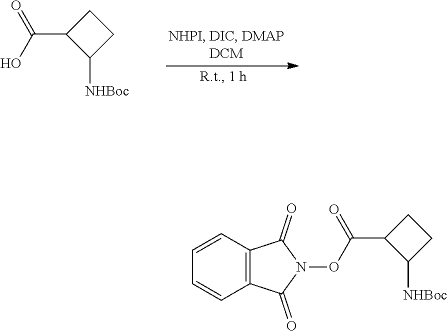 [SMILES: CC(C)(C)OC(=O)NC1CCC1C(=O)O ; CC(C)(C)OC(=O)NC1CCC1C(=O)ON1C(=O)c2ccccc2C1=O]

To a stirred mixture of 2-[(tert-butoxycarbonyl)amino]cyclobutane-1-carboxylic acid (2 g, 9.29 mmol, 1 equiv), DMAP (0.12 g, 0.93 mmol, 0.1 equiv) and NHPI (1.52 g, 9.29 mmol, 1 equiv) in DCM (30 mL) was added DIC (1.28 g, 10.22 mmol, 1.1 equiv) dropwise at room temperature under nitrogen atmosphere. The resulting mixture was stirred for 1 h at room temperature under nitrogen atmosphere. The resulting mixture was concentrated under reduced pressure. The residue was purified by silica gel column chromatography, eluted with PE/EA (3:1) to afford 1,3-dioxoisoindol-2-yl 2-[(tert-butoxycarbonyl)amino]cyclobutane-1-carboxylate (2.4 g, 71.68%) as a white solid.

### Step 2. Synthesis of 3-bromo-5-chloro-N-(thiophen-2-ylmethyl)furo[3,2-b]pyridin-7-amine

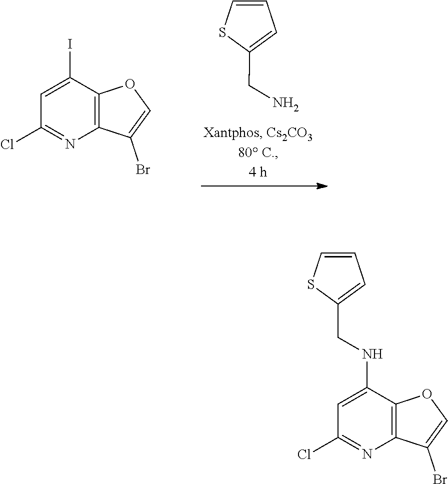 [SMILES: Clc1cc(I)c2occ(Br)c2n1 ; NCc1cccs1 ; Clc1cc(NCc2cccs2)c2occ(Br)c2n1]

To a stirred mixture of 3-bromo-5-chloro-7-iodofuro[3,2-b]pyridine (10 g, 27.905 mmol, 1 equiv) and Pd₂(dba)₃ (2.5 g, 2.730 mmol, 0.10 equiv) in Dioxane (130 mL) were added Xantphos (3.2 g, 5.530 mmol, 0.20 equiv), Cs₂CO₃ (18.2 g, 55.687 mmol, 2.00 equiv) and 1-(thiophen-2-yl)methanamine (3.2 g, 28.274 mmol, 1.01 equiv) in portions at room temperature. The resulting mixture was stirred for 4 h at 80° C. under nitrogen atmosphere. The resulting mixture was concentrated under reduced pressure. The residue was purified by silica gel column chromatography, eluted with PE/EA (3:1) to afford 3-bromo-5-chloro-N-(thiophen-2-ylmethyl)furo[3,2-b]pyridin-7-amine (8.4 g, 87.60%) as a yellow solid.

### Step 3. Synthesis of tert-butyl N-{3-bromo-5-chlorofuro[3,2-b]pyridin-7-yl}-N-(thiophen-2-ylmethyl)carbamate

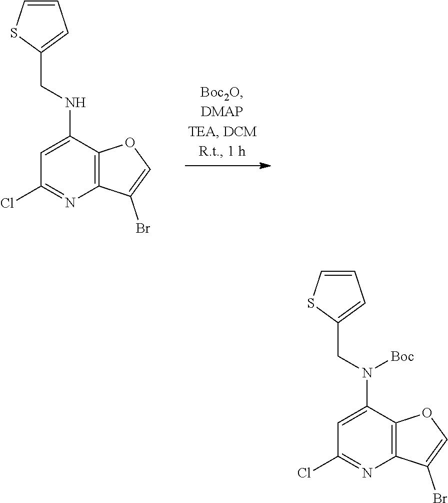 [SMILES: Clc1cc(NCc2cccs2)c2occ(Br)c2n1 ; CC(C)(C)OC(=O)N(Cc1cccs1)c1cc(Cl)nc2c(Br)coc12]

To a stirred mixture of 3-bromo-5-chloro-N-(thiophen-2-ylmethyl)furo[3,2-b]pyridin-7-amine (8.4 g, 24.446 mmol, 1 equiv) and DMAP (149 mg, 1.220 mmol, 0.05 equiv) in DCM (100 mL) was added TEA (4.9 g, 48.422 mmol, 1.98 equiv) in portions at room temperature was added di-tert-butyl dicarbonate (7.5 g, 34.364 mmol, 1.41 equiv) in portions at 0° C. under nitrogen atmosphere. The resulting mixture was stirred for 1 h at room temperature. The reaction was quenched by the addition of Water (1 mL) at room temperature. The resulting mixture was concentrated under reduced pressure. The residue was purified by silica gel column chromatography, eluted with CH₂Cl₂/PE (2:1) to afford tert-butyl N-{3-bromo-5-chlorofuro[3,2-b]pyridin-7-yl}-N-(thiophen-2-ylmethyl)carbamate (8.3 g, 76.52%) as a yellow solid.

### Step 4. Synthesis of tert-butyl N-{3-bromo-5-chloro-2-iodofuro[3,2-b]pyridin-7-yl}-N-(thiophen-2-ylmethyl) carbamate

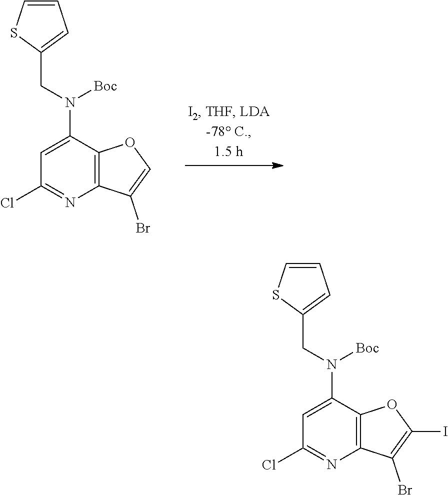 [SMILES: CC(C)(C)OC(=O)N(Cc1cccs1)c1cc(Cl)nc2c(Br)coc12 ; CC(C)(C)OC(=O)N(Cc1cccs1)c1cc(Cl)nc2c(Br)c(I)oc12]

To a stirred mixture of tert-butyl N-{3-bromo-5-chlorofuro[3,2-b]pyridin-7-yl}-N-(thiophen-2-ylmethyl) carbamate (8.3 g, 18.705 mmol, 1 equiv) in tetrahydrofuran (85 mL) in portions at room temperature was added LDA (2M in THF, 15.9 mL, 31.800 mmol, 1.70 equiv, 2 M in THF) in portions at −78° C. under nitrogen atmosphere. The resulting mixture was stirred for 20 min at −78° C. under nitrogen atmosphere. To a stirred mixture of iodine (6.2 g, 24.428 mmol, 1.31 equiv) in tetrahydrofuran (15 mL) in portions at −78° C. under nitrogen atmosphere. The resulting mixture was stirred for 1.5 h at −78° C. under nitrogen atmosphere. The residue was purified by reversed-phase flash chromatography with the following conditions: column, C18; mobile phase, MeCN in Water (0.1% FA), 10% to 50% gradient in 10 min; detector, UV 254 nm. This resulted in tert-butyl N-{3-bromo-5-chloro-2-iodofuro[3,2-b]pyridin-7-yl}-N-(thiophen-2-ylmethyl) carbamate (5.8 g, 54.43%) as a yellow solid.

### Step 5. Synthesis of tert-butyl N-(3-bromo-2-{2-[(tert-butoxycarbonyl)amino]cyclobutyl}-5-chlorofuro[3,2-b]pyridin-7-yl)-N-(thiophen-2-ylmethyl)carbamate

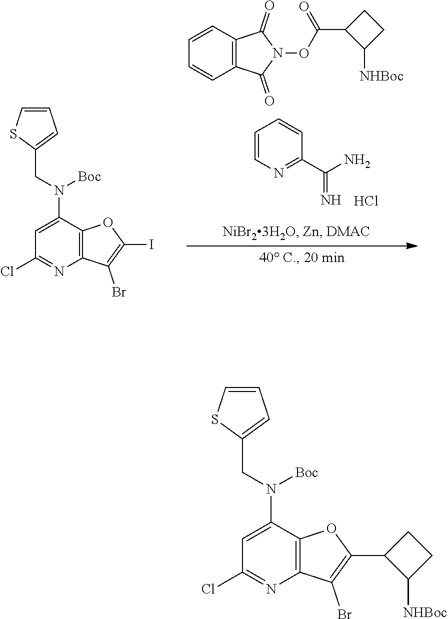

A mixture of pyridine-2-carboximidamide (0.17 g, 1.404 mmol, 0.4 equiv), Zn (275.46 mg, 4.216 mmol, 8 equiv) and nickel(II) bromide trihydrate (1.91 g, 7.022 mmol, 2 equiv) in DMAc (40 mL) was stirred for 5 min at 40° C. under nitrogen atmosphere. Then a solution of tert-butyl N-{3-bromo-5-chloro-2-iodofuro[3,2-b]pyridin-7-yl}-N-(thiophen-2-ylmethyl)carbamate (2 g, 3.511 mmol, 1 equiv) and 1,3-dioxoisoindol-2-yl 2-[(tert-butoxycarbonyl)amino]cyclobutane-1-carboxylate (1.64 g, 4.564 mmol, 1.3 equiv) in 10 mL DMAc was added dropwise and the mixture was stirred for another 20 mins at 40° C. The resulting mixture was filtered, the filter cake was washed with DMF (2×5 mL).

The filtrate was purified by reversed-phase flash chromatography with the following conditions: column, C18; mobile phase, MeCN in Water (0.1% TFA), 20% to 100% gradient in 15 min; detector, UV 254 nm. This resulted in tert-butyl N-(3-bromo-2-{2-[(tert-butoxycarbonyl)amino]cyclobutyl}-5-chlorofuro[3,2-b]pyridin-7-yl)-N-(thiophen-2-ylmethyl)carbamate (350 mg, 16.26%) as a light yellow solid.

### Step 6. Synthesis of 2-(2-aminocyclobutyl)-3-bromo-5-chloro-N-(thiophen-2-ylmethyl)furo[3,2-b]pyridin-7-amine

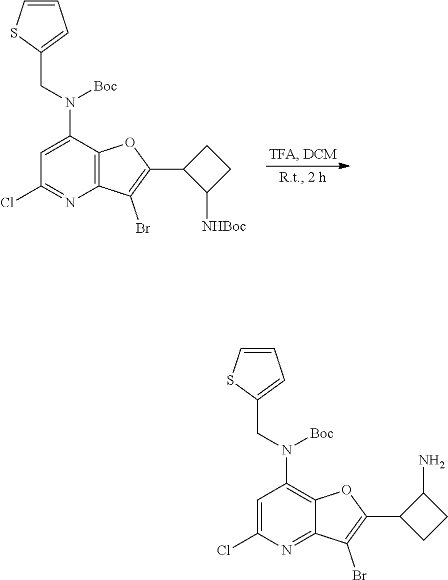 [SMILES: CC(C)(C)OC(=O)NC1CCC1c1oc2c(N(Cc3cccs3)C(=O)OC(C)(C)C)cc(Cl)nc2c1Br ; CC(C)(C)OC(=O)N(Cc1cccs1)c1cc(Cl)nc2c(Br)c(C3CCC3N)oc12]

A mixture of tert-butyl N-(3-bromo-2-{2-[(tert-butoxycarbonyl)amino]cyclobutyl}-5-chlorofuro[3,2-b]pyridin-7-yl)-N-(thiophen-2-ylmethyl)carbamate (50 mg, 0.082 mmol, 1 equiv) and TFA (1 mL) in DCM (1 mL) was stirred for 2 h at room temperature under nitrogen atmosphere. The resulting mixture was concentrated under reduced pressure. The residue was purified by reversed-phase flash chromatography with the following conditions: column, C18; mobile phase, MeCN in Water (0.1% FA), 10% to 60% gradient in 10 min; detector, UV 254 nm. This resulted in racemic trans 2-(2-aminocyclobutyl)-3-bromo-5-chloro-N-(thiophen-2-ylmethyl)furo[3,2-b]pyridin-7-amine (15 mg, 44.55%).

LC-MS (ES, m/z): [M+H]⁺=411.85.

¹H NMR (400 MHz, Methanol-d₄) δ 8.49 (s, 1H), 7.31 (dd, J=5.1, 1.2 Hz, 1H), 7.09 (dd, J=3.5, 1.2 Hz, 1H), 6.99 (dd, J=5.1, 3.5 Hz, 1H), 6.63 (s, 1H), 4.79 (s, 2H), 4.09 (q, J=8.5 Hz, 1H), 3.90 (q, J=9.1 Hz, 1H), 2.44-2.20 (m, 3H), 2.15 (q, J=9.7 Hz, 1H).

### Step 7. Chiral separation of racemic trans 2-(2-aminocyclobutyl)-3-bromo-5-chloro-N-(thiophen-2-ylmethyl)furo[3,2-b]pyridin-7-amine

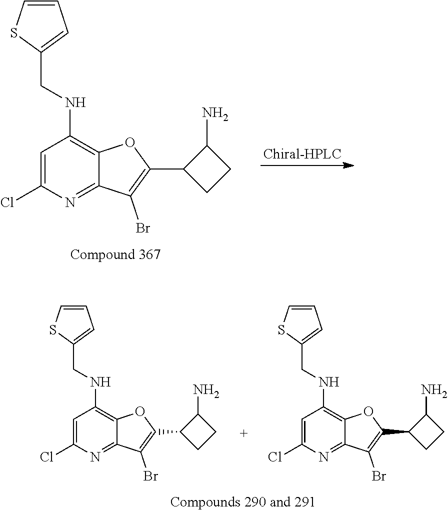 [SMILES: NC1CCC1c1oc2c(NCc3cccs3)cc(Cl)nc2c1Br ; NC1CC[C@@H]1c1oc2c(NCc3cccs3)cc(Cl)nc2c1Br ; NC1CC[C@H]1c1oc2c(NCc3cccs3)cc(Cl)nc2c1Br]

Chiral separation was performed as follows: Column: CHIRAL ART Cellulose-SZ, 3*25 cm, 5 m; Mobile Phase A: Hex (10 mM NH₃-MeOH), Mobile Phase B: EtOH-HPLC; Flow rate: 40 mL/min; Gradient: isocratic 20; Wave Length: 213/241 nm; RT1 (min): 11.4; RT2 (min): 13.573; Sample Solvent: MeOH:DCM=1:1-HPLC; Injection Volume: 0.7 mL; Number Of Runs: 6. This resulted in 2-[(1R,2R)-2-aminocyclobutyl]-3-bromo-5-chloro-N-(thiophen-2-ylmethyl)furo[3,2-b]pyridin-7-amine and 2-[(1S,2S)-2-aminocyclobutyl]-3-bromo-5-chloro-N-(thiophen-2-ylmethyl)furo[3,2-b]pyridin-7-amine. The first eluting compound is compound 291 and the second eluting compound is compound 290. The absolute configuration was not determined.

### Data for Compound 291

LC-MS: [M+H]⁺=411.90.

¹H NMR (400 MHz, Methanol-d₄) δ 7.30 (d, J=5.0 Hz, 1H), 7.07 (d, J=3.5 Hz, 1H), 6.98 (dd, J=5.1, 3.5 Hz, 1H), 6.57 (s, 1H), 4.77 (s, 2H), 3.81-3.66 (m, 1H), 3.52 (q, J=9.1 Hz, 1H), 2.38-2.18 (m, 1H), 2.16-2.00 (m, 2H), 1.87 (q, J=9.9 Hz, 1H).

### Data for Compound 290

LC-MS: [M+H]⁺=411.90.

¹H NMR (400 MHz, Methanol-d₄) δ 7.30 (d, J=5.0 Hz, 1H), 7.07 (d, J=3.5 Hz, 1H), 6.98 (dd, J=5.1, 3.5 Hz, 1H), 6.57 (s, 1H), 4.77 (s, 2H), 3.81-3.66 (m, 1H), 3.52 (q, J=9.1 Hz, 1H), 2.38-2.18 (m, 1H), 2.16-2.00 (m, 2H), 1.87 (q, J=9.9 Hz, 1H).

### Example 13: Specific Example of General Synthesis Scheme 14, Synthesis of 2-[(2R)-2-amino-4-fluorobutyl]-5-chloro-3-methyl-N-(thiophen-2-ylmethyl)furo[3,2-b]pyridin-7-amine (Compound 31)

### Step 1. Synthesis of tert-butyl N-[(2S)-4-[(tert-butyldimethylsilyl)oxy]-1-[5-chloro-3-methyl-7-(methylsulfanyl)furo[3,2-b]pyridin-2-yl]butan-2-yl]carbamate

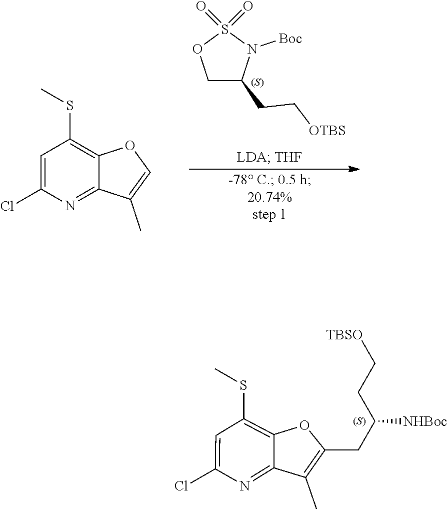

In a 50-mL round bottom flask, to a solution of 5-chloro-3-methyl-7-(methylsulfanyl)furo[3,2-b]pyridine (800 mg, 3.744 mmol, 1 equiv) in THF (10 mL) was added dropwise LDA (in 2M THF) (2.8 mL, 5.616 mmol, 1.5 equiv) at −78° C. under N₂ atmosphere. The reaction mixture was stirred at −78° C. for 30 mins. Then a solution of tert-butyl (4S)-4-{2-[(tert-butyldimethylsilyl)oxy]ethyl}-2,2-dioxo-1,2lambda6,3-oxathiazolidine-3-carboxylate (2.14 g, 5.616 mmol, 1.5 equiv) in 5 mL THE was added dropwise and the mixture was stirred for another 30 mins. The residue was purified by reversed-phase flash chromatography with the following conditions: column, C18; mobile phase, MeCN in Water (10 mmol/L NH₄HCO₃), 10% to 100% gradient in 10 min; detector, UV 254 nm. This resulted in tert-butyl N-[(2S)-4-[(tert-butyldimethylsilyl)oxy]-1-[5-chloro-3-methyl-7-(methylsulfanyl)furo[3,2-b]pyridin-2-yl]butan-2-yl]carbamate (400 mg, 20.74%) as a yellow solid.

### Step 2. Synthesis of tert-butyl N-[(2S)-1-[5-chloro-3-methyl-7-(methylsulfanyl)furo[3,2-b]pyridin-2-yl]-4-hydroxybutan-2-yl]carbamate

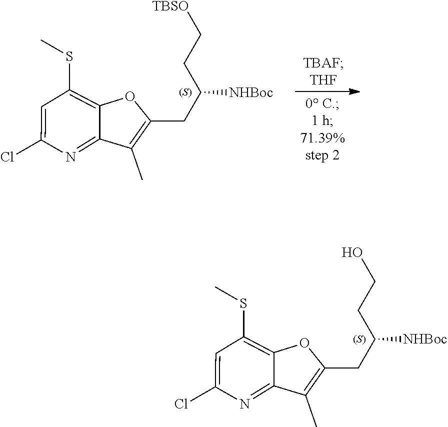

To a stirred solution of tert-butyl N-[(2S)-4-[(tert-butyldimethylsilyl)oxy]-1-[5-chloro-3-methyl-7-(methylsulfanyl)furo[3,2-b]pyridin-2-yl]butan-2-yl]carbamate (450 mg, 0.873 mmol, 1 equiv) in THE (45.00 mL) was added TBAF (228.38 mg, 0.873 mmol, 1 equiv) at 0° C. and stirred for 1 h. The reaction progress was monitored by TLC. After completion of reaction, the reaction mixture was concentrated under reduced pressure to give crude product which was further purified by column chromatography using 0 to 20 PE in EtOAc gradient to afford desired compound tert-butyl N-[(2S)-1-[5-chloro-3-methyl-7-(methylsulfanyl)furo[3,2-b]pyridin-2-yl]-4-hydroxybutan-2-yl]carbamate (250 mg, 71.39%) solid as a yellow solid.

### Step 3. Synthesis of tert-butyl N-[(2R)-1-[5-chloro-3-methyl-7-(methylsulfanyl)furo[3,2-b]pyridin-2-yl]-4-fluorobutan-2-yl]carbamate

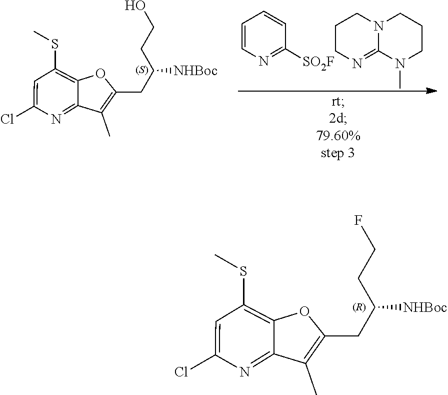

To a stirred solution of tert-butyl N-[(2S)-1-[5-chloro-3-methyl-7-(methylsulfanyl)furo[3,2-b]pyridin-2-yl]-4-hydroxybutan-2-yl]carbamate (200 mg, 0.499 mmol, 1 equiv) in toluene (20 mL) was added pyridine-2-sulfonyl fluoride (96.47 mg, 0.599 mmol, 1.2 equiv) and 1-methyl-2H,3H,4H,6H,7H,8H-pyrimido[1,2-a][1,3]diazine (76.44 mg, 0.499 mmol, 1 equiv) at room temperature and stirred for 2 days. The resulting mixture was concentrated under reduced pressure. The residue was purified by reversed-phase flash chromatography with the following conditions: column, C₁₈; mobile phase, MeCN in Water (10 mmol/L NH₄HCO₃), 10% to 100% gradient in 10 min; detector, UV 254 nm. This resulted in tert-butyl N-[(2R)-1-[5-chloro-3-methyl-7-(methylsulfanyl)furo[3,2-b]pyridin-2-yl]-4-fluorobutan-2-yl]carbamate (160 mg, 79.60%) as a yellow solid.

### Step 4. Synthesis of tert-butyl N-[(2R)-1-{5-chloro-7-methanesulfonyl-3-methylfuro[3,2-b]pyridin-2-yl}-4-fluorobutan-2-yl]carbamate

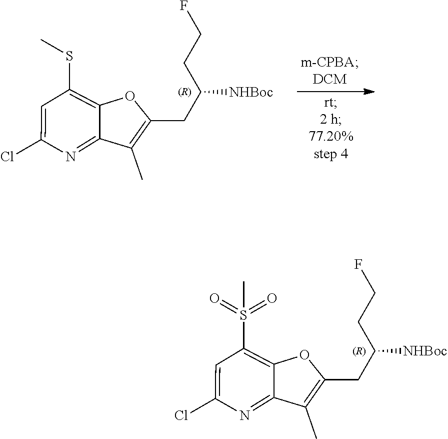

To a stirred solution of tert-butyl N-[(2R)-1-[5-chloro-3-methyl-7-(methylsulfanyl)furo[3,2-b]pyridin-2-yl]-4-fluorobutan-2-yl]carbamate (180 mg, 0.447 mmol, 1 equiv) in DCM (10 mL) was added m-CPBA (231.27 mg, 1.341 mmol, 3 equiv 85%) at room temperature and stirred for 2 h. The residue was purified by silica gel column chromatography, eluted with PE/EA (4:1) to afford tert-butyl N-[(2R)-1-{5-chloro-7-methanesulfonyl-3-methylfuro[3,2-b]pyridin-2-yl}-4-fluorobutan-2-yl]carbamate (150 mg, 77.20%) as a white solid.

### Step 5. Synthesis of tert-butyl N-[(2R)-1-{5-chloro-3-methyl-7-[(thiophen-2-ylmethyl)amino]furo[3,2-b]pyridin-2-yl}-4-fluorobutan-2-yl]carbamate

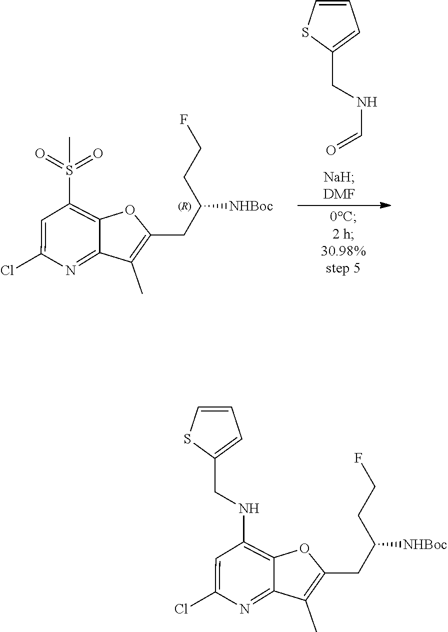

To a stirred solution of tert-butyl N-[(2R)-1-{5-chloro-7-methanesulfonyl-3-methylfuro[3,2-b]pyridin-2-yl}-4-fluorobutan-2-yl]carbamate (150 mg, 0.345 mmol, 1 equiv) and N-(thiophen-2-ylmethyl)formamide (73.04 mg, 0.517 mmol, 1.5 equiv) in DMF (7.50 mL) was added NaH (60%) (24.83 mg, 1.035 mmol, 3 equiv) at 0° C. and stirred for 2 h. The reaction was quenched with Water (1 mL) at 0° C. The residue was purified by reversed-phase flash chromatography with the following conditions: column, C₁₈; mobile phase, MeCN in Water (10 mmol/L NH₄HCO₃), 10% to 100% gradient in 10 min; detector, UV 254 nm. This resulted in tert-butyl N-[(2R)-1-{5-chloro-3-methyl-7-[(thiophen-2-ylmethyl)amino]furo[3,2-b]pyridin-2-yl}-4-fluorobutan-2-yl]carbamate (50 mg, 30.98%) as a yellow solid.

### Step 6. Synthesis of 2-[(2R)-2-amino-4-fluorobutyl]-5-chloro-3-methyl-N-(thiophen-2-ylmethyl)furo[3,2-b]pyridin-7-amine

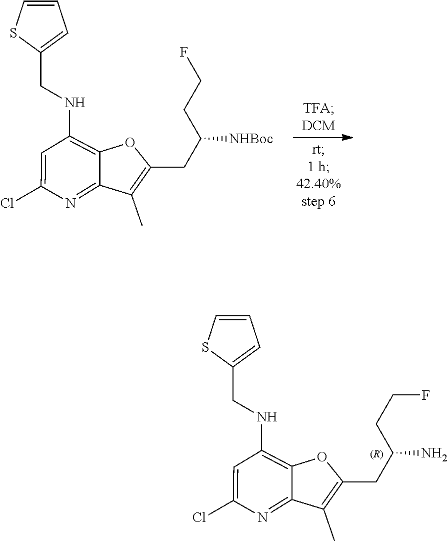

Into a 8 mL vial were added tert-butyl N-[(2R)-1-{5-chloro-3-methyl-7-[(thiophen-2-ylmethyl)amino]furo[3,2-b]pyridin-2-yl}-4-fluorobutan-2-yl]carbamate (60 mg, 0.128 mmol, 1 equiv), DCM (1.5 mL) and TFA (0.5 mL) at 0° C. The resulting mixture was stirred for 1 h at room temperature. The resulting mixture was concentrated under vacuum.

The residue was purified by reversed-phase flash chromatography with the following conditions: column, C₁₈; mobile phase, MeCN in Water (0.1% TFA), 10% to 50% gradient in 10 min; detector, UV 254 nm. This resulted in 2-[(2R)-2-amino-4-fluorobutyl]-5-chloro-3-methyl-N-(thiophen-2-ylmethyl)furo[3,2-b]pyridin-7-amine (20 mg, 42.40%).

LC-MS (ES, m/z): [M+H]⁺=368.

¹H NMR (400 MHz, Methanol-d₄) δ 7.31 (dd, J=5.1, 1.2 Hz, 1H), 7.08 (dq, J=3.2, 1.0 Hz, 1H), 6.98 (dd, J=5.1, 3.5 Hz, 1H), 6.61 (s, 1H), 4.80-4.77 (m, 2H), 4.75-4.67 (m, 1H), 4.66-4.53 (m, 1H), 3.84 (p, J=6.5 Hz, 1H), 3.21 (p, J=8.9 Hz, 2H), 2.20 (s, 3H), 2.17-2.00 (m, 2H).

### Example 14: Specific Example of General Method 15, Synthesis of 2-[(1-aminocyclopropyl)methyl]-3-bromo-5-chloro-N-(thiophen-2-ylmethyl)furo[3,2-b]pyridin-7-amine (Compound 227)

### Step 1. Synthesis of benzyl N-[1-((3-bromo-5-chloro-7-iodofuro[3,2-b]pyridin-2-ylmethyl)cyclopropyl]carbamate

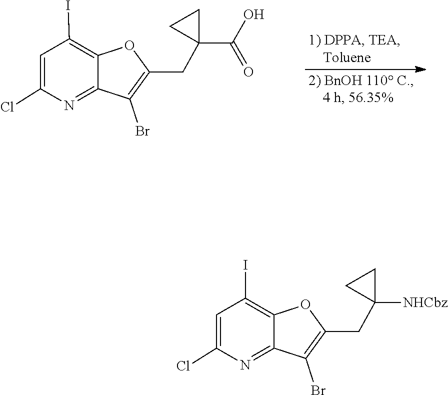 [SMILES: O=C(O)C1(Cc2oc3c(I)cc(Cl)nc3c2Br)CC1 ; O=C(NC1(Cc2oc3c(I)cc(Cl)nc3c2Br)CC1)OCc1ccccc1]

Into a 40-mL vial were added 1-((3-bromo-5-chloro-7-iodofuro[3,2-b]pyridin-2-ylmethyl)cyclopropane-1-carboxylic acid (750 mg, 1.643 mmol, 1 equiv), DPPA (678.27 mg, 2.465 mmol, 1.5 equiv), TEA (498.80 mg, 4.929 mmol, 3 equiv) and Toluene (8 mL) at room temperature. The resulting solution was stirred for 30 mins at 85° C. The mixture was allowed to cool down to room temperature. To the above mixture was added phenylmethanol (4 mL).

The resulting mixture was stirred for additional 4 h at 110° C. The resulting mixture was concentrated under vacuum. The crude product was purified by Prep-HPLC with the following conditions (C18, Mobile Phase A: Water/0.1% FA, Mobile Phase B: Acetonitrile, 35-100%, 16 min). This resulted in benzyl N-[1-((3-bromo-5-chloro-7-iodofuro[3,2-b]pyridin-2-ylmethyl)cyclopropyl]carbamate (520 mg, 56.35%) as a yellow solid.

### Step 2. Synthesis of benzyl N-[1-((3-bromo-5-chloro-7-[(thiophen-2-ylmethyl)amino]furo[3,2-b]pyridin-2-ylmethyl)cyclopropyl]carbamate

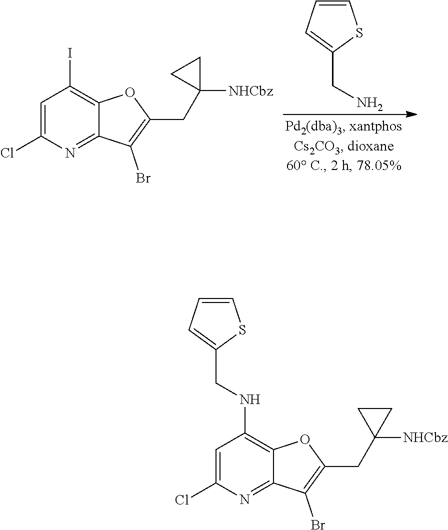 [SMILES: O=C(NC1(Cc2oc3c(I)cc(Cl)nc3c2Br)CC1)OCc1ccccc1 ; NCc1cccs1 ; O=C(NC1(Cc2oc3c(NCc4cccs4)cc(Cl)nc3c2Br)CC1)OCc1ccccc1]

Into a 8-mL vial purged and maintained with an inert atmosphere of nitrogen were added benzyl benzyl N-[1-((3-bromo-5-chloro-7-iodofuro[3,2-b]pyridin-2-ylmethyl)cyclopropyl]carbamate (250 mg, 0.445 mmol, 1 equiv), 1-(thiophen-2-yl)methanamine (75.57 mg, 0.667 mmol, 1.5 equiv), Pd₂(dba)₃ (20.38 mg, 0.022 mmol, 0.05 equiv), xantphos (25.76 mg, 0.045 mmol, 0.1 equiv), Cs₂CO₃ (290.08 mg, 0.890 mmol, 2 equiv) and dioxane (3 mL) at room temperature. The resulting solution was stirred for 2 hr at 60° C. The crude product was purified by Prep-HPLC with the following conditions (C₁₈, Mobile Phase A: Water/0.1% FA, Mobile Phase B: Acetonitrile, 25-100%, 16 min). This resulted in 190 mg (78.05%) of benzyl N-[1-((3-bromo-5-chloro-7-[(thiophen-2-ylmethyl)amino]furo[3,2-b]pyridin-2-ylmethyl)cyclopropyl]carbamate as a yellow solid.

### Step 3. Synthesis of 2-[(1-aminocyclopropyl)methyl]-3-bromo-5-chloro-N-(thiophen-2-ylmethyl)furo[3,2-b]pyridin-7-amine

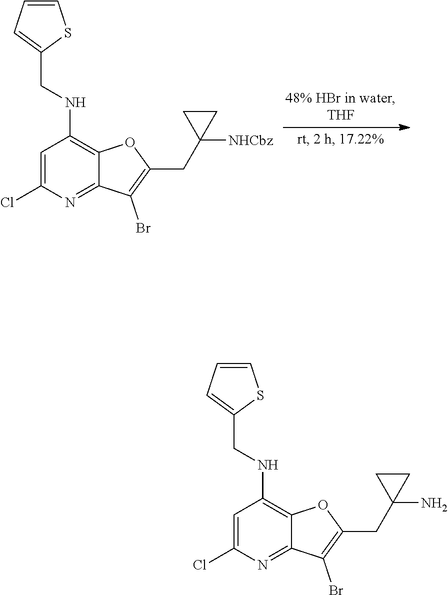 [SMILES: O=C(NC1(Cc2oc3c(NCc4cccs4)cc(Cl)nc3c2Br)CC1)OCc1ccccc1 ; NC1(Cc2oc3c(NCc4cccs4)cc(Cl)nc3c2Br)CC1]

Into a 8-mL vial were added benzyl N-[1-((3-bromo-5-chloro-7-[(thiophen-2-ylmethyl)amino]furo[3,2-b]pyridin-2-ylmethyl)cyclopropyl]carbamate (190 mg, 0.347 mmol, 1 equiv), HBr in water (2 mL, 49%) and THF (0.2 mL) at 0° C. The resulting solution was stirred for 2 hr at room temperature. The crude product was purified by Prep-HPLC with the following conditions (AQ-C18, Mobile Phase A: Water/0.1% FA, Mobile Phase B: Acetonitrile, 25-90%, 20 min). This resulted in 25 mg (17.22%) of 2-[(1-aminocyclopropyl)methyl]-3-bromo-5-chloro-N-(thiophen-2-ylmethyl)furo[3,2-b]pyridin-7-amine.

LCMS: (ES, m/z): [M+H]⁺=411.80

¹H NMR (400 MHz, Methanol-d₄) δ 8.39 (s, 1H), 7.32 (dd, J=5.1, 1.2 Hz, 1H), 7.12-7.06 (m, 1H), 6.98 (dd, J=5.1, 3.4 Hz, 1H), 6.65 (s, 1H), 3.26 (s, 2H), 1.04 (t, J=6.6 Hz, 2H), 1.00-0.93 (m, 2H).

### Example 15: Specific Example of General Method 16, Synthesis of 2-[(2R,3S)-2-amino-3-fluorobutyl]-5-chloro-7-[(thiophen-2-ylmethyl)amino]furo[3,2-b]pyridine-3-carbonitrile Compound 237

### Step 1. Synthesis of tert-butyl N-[(2R,3S)-1-{5-chloro-3-ethenyl-7-[(thiophen-2-ylmethyl)amino]furo[3,2-b]pyridin-2-yl}-3-fluorobutan-2-yl]carbamate

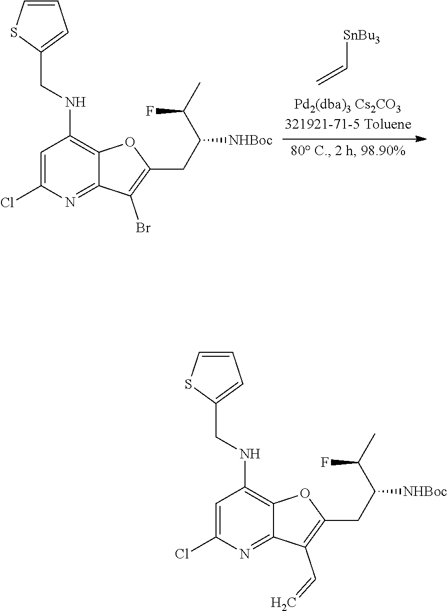

To a stirred mixture of tert-butyl N-[(2R,3S)-1-{3-bromo-5-chloro-7-[(thiophen-2-ylmethyl) amino]furo[3,2-b]pyridin-2-yl}-3-fluorobutan-2-yl]carbamate (440 mg, 0.826 mmol, 1 equiv) and Cs₂CO₃ (540 mg, 1.657 mmol, 2.01 equiv) in Toluene (22 mL) were added Pd₂(dba)₃ (76 mg, 0.083 mmol, 0.10 equiv) and butylbis[(3R,5S,7s)-adamantan-1-yl]phosphane (59 mg, 0.165 mmol, 0.20 equiv), tributyl(ethenyl)stannane (264 mg, 0.833 mmol, 1.01 equiv) in portions at room temperature. The resulting mixture was stirred for 2 h at 80° C. under nitrogen atmosphere. The resulting mixture was concentrated under reduced pressure.

The residue was purified by reversed-phase flash chromatography with the following conditions: column, C₁₈; mobile phase, MeCN in Water (0.1% FA), 10% to 50% gradient in 10 min; detector, UV 254 nm. This resulted in tert-butyl N-[(2R,3S)-1-{5-chloro-3-ethenyl-7-[(thiophen-2-ylmethyl)amino]furo[3,2-b]pyridin-2-yl}-3-fluorobutan-2-yl]carbamate (392 mg, 98.90%) as a brown solid.

### Step 2. Synthesis of tert-butyl N-[(2R,3S)-1-{5-chloro-3-formyl-7-[(thiophen-2-ylmethyl)amino]furo[3,2-b]pyridin-2-yl}-3-fluorobutan-2-yl]carbamate

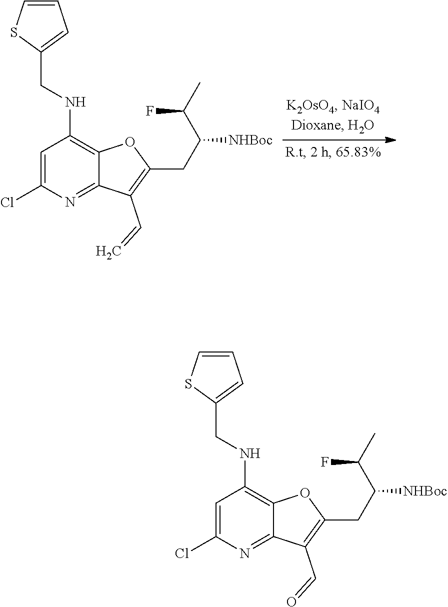 [SMILES: C=Cc1c(C[C@@H](NC(=O)OC(C)(C)C)[C@H](C)F)oc2c(NCc3cccs3)cc(Cl)nc12 ; C[C@H](F)[C@@H](Cc1oc2c(NCc3cccs3)cc(Cl)nc2c1C=O)NC(=O)OC(C)(C)C]

To a stirred mixture of tert-butyl N-[(2R,3S)-1-{5-chloro-3-ethenyl-7-[(thiophen-2-ylmethyl) amino]furo[3,2-b]pyridin-2-yl}-3-fluorobutan-2-yl]carbamate (410 mg, 0.854 mmol, 1 equiv) and K₂OsO₄-2H₂O (63 mg, 0.171 mmol, 0.20 equiv) in Dioxane (6 mL) were added water (1.5 mL, 0.083 mmol, 0.10 equiv) and NaIO₄ (1.47 g, 6.873 mmol, 8.05 equiv) in portions at room temperature. The resulting mixture was stirred for 2 h at room temperature under nitrogen atmosphere. The residue was purified by reversed-phase flash chromatography with the following conditions: column, C₁₈; mobile phase, MeCN in Water (0.1% FA), 10% to 50% gradient in 10 min; detector, UV 254 nm. This resulted in tert-butyl N-[(2R,3S)-1-{5-chloro-3-formyl-7-[(thiophen-2-ylmethyl)amino]furo[3,2-b]pyridin-2-yl}-3-fluorobutan-2-yl]carbamate (271 mg, 65.83%) as a yellow solid.

### Step 3. Synthesis of tert-butyl N-[(2R,3S)-1-{5-chloro-3-[(1Z)-(hydroxyimino)methyl]-7-[(thiophen-2-ylmethyl)amino]furo[3,2-b]pyridin-2-yl}-3-fluorobutan-2-yl]carbamate

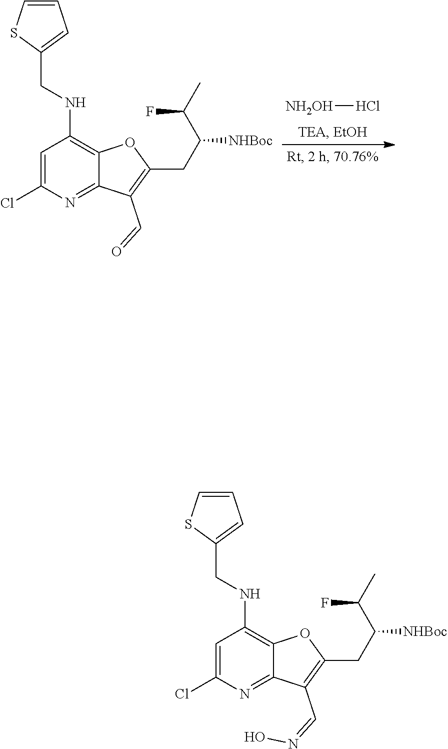

To a stirred mixture of tert-butyl N-[(2R,3S)-1-{5-chloro-3-formyl-7-[(thiophen-2-ylmethyl)amino]furo[3,2-b]pyridin-2-yl}-3-fluorobutan-2-yl]carbamate (270 mg, 0.560 mmol, 1 equiv) and hydroxylamine hydrochloride (59 mg, 0.849 mmol, 1.52 equiv) in ethyl alcohol (6 mL) was added TEA (170 mg, 1.680 mmol, 3.00 equiv) in portions at room temperature. The resulting mixture was stirred for 2 h at room temperature under nitrogen atmosphere. The resulting mixture was concentrated under reduced pressure. The residue was dissolved in DCM (5 mL). The residue was purified by silica gel column chromatography, eluted with PE/EA (2:1) to afford tert-butyl N-[(2R,3S)-1-{5-chloro-3-[(1Z)-(hydroxyimino)methyl]-7-[(thiophen-2-ylmethyl)amino]furo[3,2-b]pyridin-2-yl}-3-fluorobutan-2-yl]carbamate (197 mg, 70.76%) as a yellow solid.

### Step 4. Synthesis of tert-butyl N-[(2R,3S)-1-{5-chloro-3-cyano-7-[(thiophen-2-ylmethyl)amino]furo[3,2-b]pyridin-2-yl}-3-fluorobutan-2-yl]carbamate

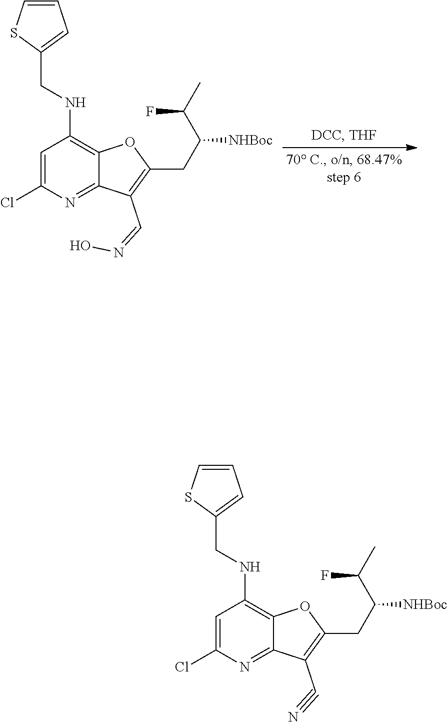 [SMILES: C[C@H](F)[C@@H](Cc1oc2c(NCc3cccs3)cc(Cl)nc2c1/C=N\O)NC(=O)OC(C)(C)C ; C[C@H](F)[C@@H](Cc1oc2c(NCc3cccs3)cc(Cl)nc2c1C#N)NC(=O)OC(C)(C)C]

To a stirred mixture of tert-butyl N-[(2R,3S)-1-{5-chloro-3-[(1Z)-(hydroxyimino)methyl]-7-[(thiophen-2-ylmethyl)amino]furo[3,2-b]pyridin-2-yl}-3-fluorobutan-2-yl]carbamate (197 mg, 0.396 mmol, 1 equiv) in tetrahydrofuran (4 mL) was added DCC (328 mg, 1.590 mmol, 4.01 equiv) in portions at room temperature. The resulting mixture was stirred for overnight at 70° C. under nitrogen atmosphere. The residue was purified by reversed-phase flash chromatography with the following conditions: column, C₁₈; mobile phase, MeCN in Water (0.1% FA), 10% to 50% gradient in 10 min; detector, UV 254 nm. This resulted in tert-butyl N-[(2R,3S)-1-{5-chloro-3-cyano-7-[(thiophen-2-ylmethyl)amino]furo[3,2-b]pyridin-2-yl}-3-fluorobutan-2-yl]carbamate (130 mg, 68.47%) as a yellow solid.

### Step 5. Synthesis of 2-[(2R,3S)-2-amino-3-fluorobutyl]-5-chloro-7-[(thiophen-2-ylmethyl)amino]furo[3,2-b]pyridine-3-carbonitrile

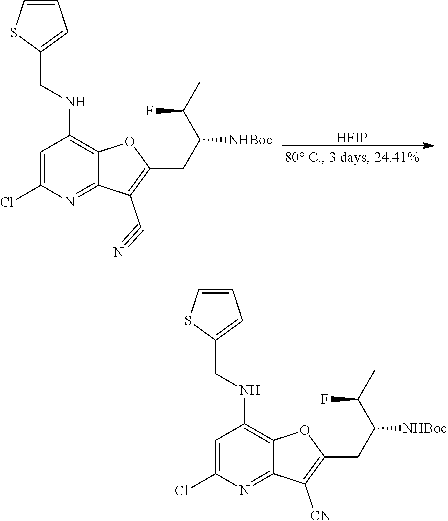 [SMILES: C[C@H](F)[C@@H](Cc1oc2c(NCc3cccs3)cc(Cl)nc2c1C#N)NC(=O)OC(C)(C)C ; "HFIP" ; C[C@H](F)[C@@H](Cc1oc2c(NCc3cccs3)cc(Cl)nc2c1C#N)NC(=O)OC(C)(C)C]

To a stirred mixture of tert-butyl N-[(2R,3S)-1-{5-chloro-3-cyano-7-[(thiophen-2-ylmethyl)amino]furo[3,2-b]pyridin-2-yl}-3-fluorobutan-2-yl]carbamate (130 mg, 0.271 mmol, 1 equiv) in 1,1,1,3,3,3-hexafluoropropan-2-ol (2 mL) in portions at room temperature. The resulting mixture was stirred for 3 days at 80° C. under nitrogen atmosphere. The resulting mixture was concentrated under reduced pressure. The residue was dissolved in DMF (1.5 mL). The residue was purified by reversed-phase flash chromatography with the following conditions: column, C₁₈; mobile phase, MeCN in Water (10 mmol/L NH₄HCO₃), 10% to 50% gradient in 10 min; detector, UV 254 nm. This resulted in 2-[(2R,3S)-2-amino-3-fluorobutyl]-5-chloro-7-[(thiophen-2-ylmethyl)amino]furo[3,2-b]pyridine-3-carbonitrile (25.1 mg, 24.41%).

LC-MS (ES, m/z): [M+H]⁺=378.90.

¹H NMR (300 MHz, Methanol-d₄) δ 7.33 (dd, J=5.1, 1.3 Hz, 1H), 7.11 (dq, J=3.3, 1.0 Hz, 1H), 7.00 (dd, J=5.1, 3.5 Hz, 1H), 6.64 (s, 1H), 4.79 (d, J=1.0 Hz, 2H), 4.70 (q, J=6.1 Hz, OH), 3.98 (dd, J=15.3, 5.3 Hz, 1H), 1.45 (dd, J=24.2, 6.3 Hz, 3H).

### Example 16: Synthesis of 2-[(2S)-2-aminopropyl]-3-bromo-7-[(thiophen-2-ylmethyl)amino]furo[3,2-b]pyridine-5-carboxylic acid (Compound 167)

### Step 1. Synthesis of 3-bromo-2-[(2S)-2-[(tert-butoxycarbonyl)amino]propyl]-7-[(thiophen-2-ylmethyl)amino]furo[3,2-b]pyridine-5-carboxylic acid

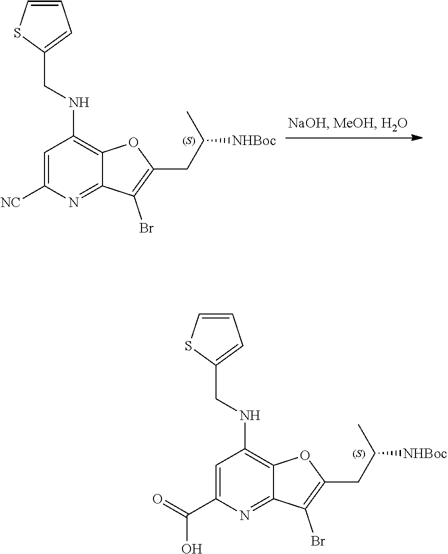

Into a 8 mL vial were added tert-butyl N-[(2S)-1-{3-bromo-5-cyano-7-[(thiophen-2-ylmethyl)amino]furo[3,2-b]pyridin-2-yl}propan-2-yl]carbamate (150 mg, 0.305 mmol, 1 equiv), MeOH (3 mL), H₂O (1 mL) and NaOH (36.63 mg, 0.915 mmol, 3 equiv) at room temperature. The resulting mixture was stirred for overnight at 60° C. The resulting mixture was concentrated under reduced pressure. The residue was purified by reversed-phase flash chromatography with the following conditions: column, C₁₈; mobile phase, MeCN in Water (10 mmol/L NH₄HCO₃), 10% to 50% gradient in 10 min; detector, UV 254 nm. This resulted in 3-bromo-2-[(2S)-2-[(tert-butoxycarbonyl)amino]propyl]-7-[(thiophen-2-ylmethyl)amino]furo[3,2-b]pyridine-5-carboxylic acid (20 mg, 12.84%) as a white solid.

### Step 2. Synthesis of 2-[(2S)-2-aminopropyl]-3-bromo-7-[(thiophen-2-ylmethyl)amino]furo[3,2-b]pyridine-5-carboxylic acid

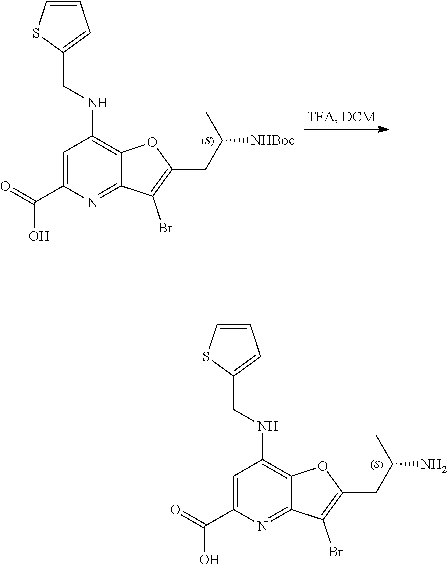

Into a 8 mL vial were added 3-bromo-2-[(2S)-2-[(tert-butoxycarbonyl)amino]propyl]-7-[(thiophen-2-ylmethyl)amino]furo[3,2-b]pyridine-5-carboxylic acid (20 mg, 0.039 mmol, 1 equiv), DCM (3 mL) and TFA (1 mL) at room temperature. The resulting mixture was stirred for 1 h at room temperature The resulting mixture was concentrated under reduced pressure. The residue was purified by reversed-phase flash chromatography with the following conditions: column, C₁₈; mobile phase, MeCN in Water (0.1% FA), 10% to 50% gradient in 10 min; detector, UV 254 nm. This resulted in 2-[(2S)-2-aminopropyl]-3-bromo-7-[(thiophen-2-ylmethyl)amino]furo[3,2-b]pyridine-5-carboxylic acid (8 mg, 49.76%).

LCMS (ES, m/z): [M+H]⁺=409.8

¹H NMR (300 MHz, Methanol-d₄) δ 7.50 (s, 1H), 7.34 (dd, J=5.1, 1.1 Hz, 1H), 7.15 (d, J=3.2 Hz, 1H), 7.00 (dd, J=5.1, 3.5 Hz, 1H), 3.89 (d, J=7.4 Hz, 1H), 3.27 (q, J=8.5, 7.2 Hz, 2H), 1.44 (d, J=6.5 Hz, 3H).

### Example 17: Synthesis of N-[(2R)-2-amino-3-(3-bromo-5-chloro-7-[(thiophen-2-ylmethyl)amino]furo[3,2-b]pyridin-2-ylpropyl]acetamide (Compound 219)

### Step 1. Synthesis of tert-butyl N-[(2R)-1-azido-3-(3-bromo-5-chloro-7-iodofuro[3,2-b]pyridin-2-ylpropan-2-yl]carbamate

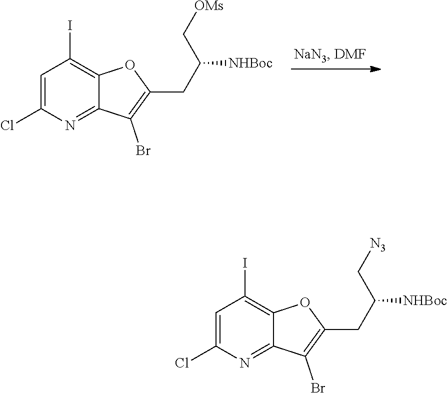 [SMILES: CC(C)(C)OC(=O)N[C@@H](COS(C)(=O)=O)Cc1oc2c(I)cc(Cl)nc2c1Br ; CC(C)(C)OC(=O)N[C@@H](CN=[N+]=[N-])Cc1oc2c(I)cc(Cl)nc2c1Br]

Into a 8-mL vial purged and maintained with an inert atmosphere of nitrogen were added tert-butyl N-[(2R)-1-(3-bromo-5-chloro-7-iodofuro[3,2-b]pyridin-2-yl-3-(methanesulfonyloxy)propan-2-yl]carbamate (300 mg, 0.492 mmol, 1 equiv), NaN₃ (31.99 mg, 0.492 mmol, 1 equiv) and DMF (3 mL) at room temperature. The resulting solution was stirred for 2 hr at 35° C. After completion of reaction, the reaction mixture was quenched by addition of water (30 mL). The aqueous layer was extracted with EA (3×30 mL). The combined organic phase was washed with brine (3×30 mL), dried over anhydrous sodium sulfate and concentrated under reduced pressure to give crude product. The crude product was purified by reversed-phase flash chromatography with the following conditions: column, C₁₈; mobile phase, ACN in 0.1% NH₄HCO₃, 25% to 100% gradient in 15 min; detector, UV 254 nm. This resulted in 130 mg (47.47%) of tert-butyl N-[(2R)-1-azido-3-(3-bromo-5-chloro-7-iodofuro[3,2-b]pyridin-2-ylpropan-2-yl]carbamate (130 mg, 47.47%) as an off-white solid.

### Step 2. Synthesis of tert-butyl N-[(2R)-1-amino-3-(3-bromo-5-chloro-7-iodofuro[3,2-b]pyridin-2-ylpropan-2-yl]carbamate

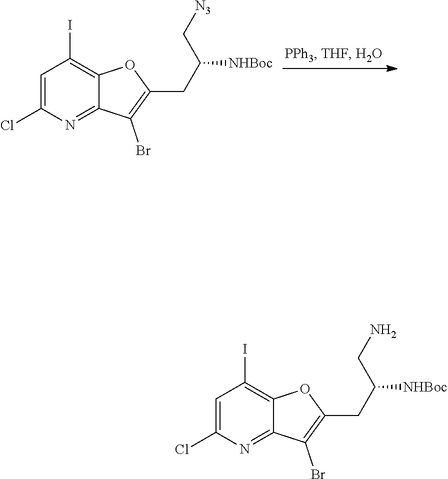 [SMILES: CC(C)(C)OC(=O)N[C@@H](CN=[N+]=[N-])Cc1oc2c(I)cc(Cl)nc2c1Br ; CC(C)(C)OC(=O)N[C@@H](CN)Cc1oc2c(I)cc(Cl)nc2c1Br]

To a solution of tert-butyl N-[(2R)-1-azido-3-(3-bromo-5-chloro-7-iodofuro[3,2-b]pyridin-2-ylpropan-2-yl]carbamate (130 mg, 0.234 mmol, 1 equiv) in THF (1 mL) was added PPh₃ (183.79 mg, 0.702 mmol, 3 equiv) and H₂O (42.08 mg, 2.340 mmol, 10 equiv).

The mixture was stirred at room temperature overnight. The reaction was quenched with NH₄Cl (30 mL) and extracted with EA (3×30 mL). The combined organic phase was washed with brine (3×30 mL), dried over anhydrous sodium sulfate and concentrated under reduced pressure to give crude product. The crude product was purified by reversed-phase flash chromatography with the following conditions: column, C₁₈; mobile phase, ACN in 0.1% NH₄HCO₃, 10% to 50% gradient in 15 min; detector, UV 254 nm. This resulted in 70 mg (56.48%) of tert-butyl N-[(2R)-1-amino-3-(3-bromo-5-chloro-7-iodofuro[3,2-b]pyridin-2-ylpropan-2-yl]carbamate (70 mg, 56.48%) as an off-white solid.

### Step 3. Synthesis of tert-butyl N-[(2R)-1-(3-bromo-5-chloro-7-iodofuro[3,2-b]pyridin-2-yl-3-acetamidopropan-2-yl]carbamate

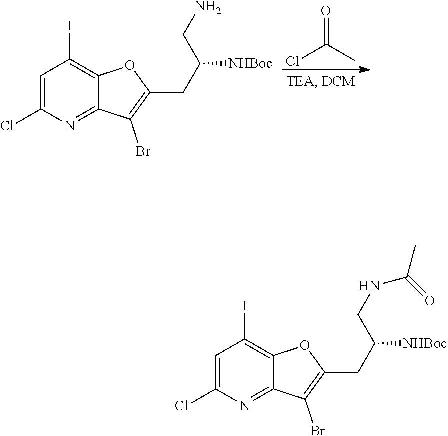

To a stirred solution of tert-butyl N-[(2R)-1-amino-3-(3-bromo-5-chloro-7-iodofuro[3,2-b]pyridin-2-ylpropan-2-yl]carbamate (70 mg, 0.132 mmol, 1 equiv) and TEA (26.70 mg, 0.264 mmol, 2 equiv) in DCM (1 mL) was added acetyl chloride (10.36 mg, 0.132 mmol, 1 equiv) at 0° C. The reaction mixture was stirred at room temperature for a period of 0.5 hr. After completion of reaction, the reaction mixture was quenched by addition of water (10 mL). The aqueous layer was extracted with DCM (3×10 mL). The combined organic phase was washed with brine (3×10 mL), dried over anhydrous sodium sulfate and concentrated under reduced pressure to give crude product. The crude product was purified by silica gel column chromatography, eluted with EA/PE (1:10) to afford tert-butyl N-[(2R)-1-(3-bromo-5-chloro-7-iodofuro[3,2-b]pyridin-2-yl-3-acetamidopropan-2-yl]carbamate (70 mg, 92.66%) as a yellow semi-solid.

### Step 4. Synthesis of tert-butyl N-[(2R)-1-(3-bromo-5-chloro-7-[(thiophen-2-ylmethyl)amino]furo[3,2-b]pyridin-2-yl-3-acetamidopropan-2-yl]carbamate

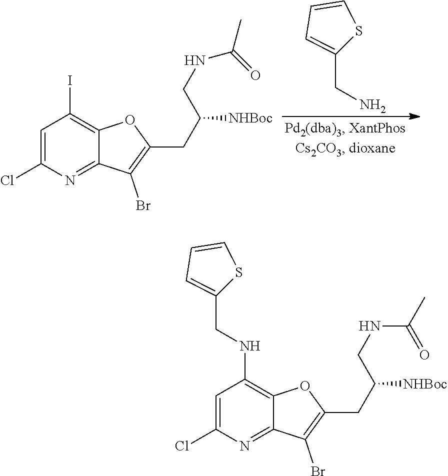 [SMILES: CC(=O)NC[C@@H](Cc1oc2c(I)cc(Cl)nc2c1Br)NC(=O)OC(C)(C)C ; NCc1cccs1 ; CC(=O)NC[C@@H](Cc1oc2c(NCc3cccs3)cc(Cl)nc2c1Br)NC(=O)OC(C)(C)C]

Into a 8-mL vial purged and maintained with an inert atmosphere of nitrogen were added tert-butyl N-[(2R)-1-(3-bromo-5-chloro-7-iodofuro[3,2-b]pyridin-2-yl-3-acetamidopropan-2-yl]carbamate (70 mg, 0.122 mmol, 1 equiv), 1-(thiophen-2-yl)methanamine (20.75 mg, 0.183 mmol, 1.5 equiv), Pd₂(dba)₃ (5.60 mg, 0.006 mmol, 0.05 equiv), XantPhos (10.61 mg, 0.018 mmol, 0.15 equiv), Cs₂CO₃ (79.66 mg, 0.244 mmol, 2 equiv) and dioxane (1 mL) at room temperature. The resulting solution was stirred for 6 hr at 60° C. The crude product was purified by reversed-phase flash chromatography with the following conditions: column, C₁₈; mobile phase, ACN in 0.1% FA, 25% to 100% gradient in 15 min; detector, UV 254 nm. This resulted in 50 mg (73.31%) of tert-butyl N-[(2R)-1-(3-bromo-5-chloro-7-[(thiophen-2-ylmethyl)amino]furo[3,2-b]pyridin-2-yl-3-acetamidopropan-2-yl]carbamate (50 mg, 73.31%) as a light yellow solid.

### Step 5. Synthesis of N-[(2R)-2-amino-3-(3-bromo-5-chloro-7-[(thiophen-2-ylmethyl)amino]furo[3,2-b]pyridin-2-ylpropyl]acetamide

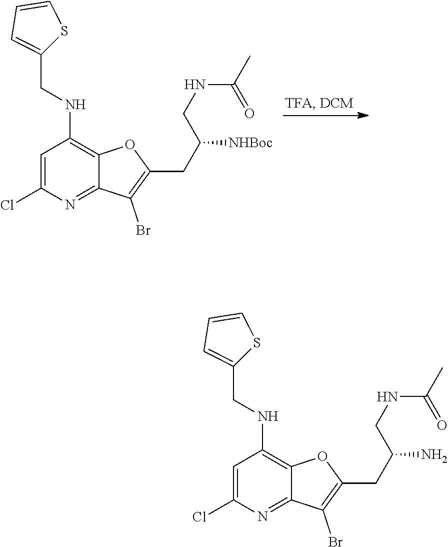 [SMILES: CC(=O)NC[C@@H](Cc1oc2c(NCc3cccs3)cc(Cl)nc2c1Br)NC(=O)OC(C)(C)C ; CC(=O)NC[C@H](N)Cc1oc2c(NCc3cccs3)cc(Cl)nc2c1Br]

Into a 8-mL vial were added tert-butyl N-[(2R)-1-(3-bromo-5-chloro-7-[(thiophen-2-ylmethyl)amino]furo[3,2-b]pyridin-2-yl-3-acetamidopropan-2-yl]carbamate (50 mg, 0.090 mmol, 1 equiv), TFA (2 mL) and DCM (1 mL) at room temperature. The resulting solution was stirred for 0.5 h at room temperature. The resulting mixture was concentrated under vacuum. The mixture was neutralized to pH 8 with NH₃. The crude product was purified by Prep-HPLC with the following conditions (Column: CHIRAL ART Cellulose-SZ, 4.6*50 mm, 3 μm; Mobile Phase A: Hex (0.1% DEA):EtOH=80:20; Flow rate: 1.67 ml/min mL/min; Gradient: isocratic; Injection Volume: 3 mL) to afford N-[(2R)-2-amino-3-(3-bromo-5-chloro-7-[(thiophen-2-ylmethyl)amino]furo[3,2-b]pyridin-2-ylpropyl]acetamide (10 mg, 24.30%).

LC-MS (ES, m/z): [M+H]⁺=458.0

¹H NMR (400 MHz, DMSO-d₆) δ 7.93 (dt, J=15.5, 5.9 Hz, 2H), 7.40 (dd, J=5.0, 1.3 Hz, 1H), 7.13-7.07 (m, 1H), 6.98 (dd, J=5.1, 3.4 Hz, 1H), 6.61 (s, 1H), 4.71 (d, J=6.1 Hz, 2H), 3.22-3.08 (m, 3H), 3.00 (dt, J=12.9, 6.3 Hz, 1H), 2.90 (dd, J=14.9, 5.0 Hz, 1H), 2.75 (dd, J=14.8, 8.2 Hz, 1H), 1.82 (s, 3H).

### Example 18: Synthesis of 1-[(2S)-2-amino-3-{5-chloro-3-ethynyl-7-[(thiophen-2-ylmethyl)amino]furo[3,2-b]pyridin-2-yl}propyl]cyclopropan-1-ol (Compound 281)

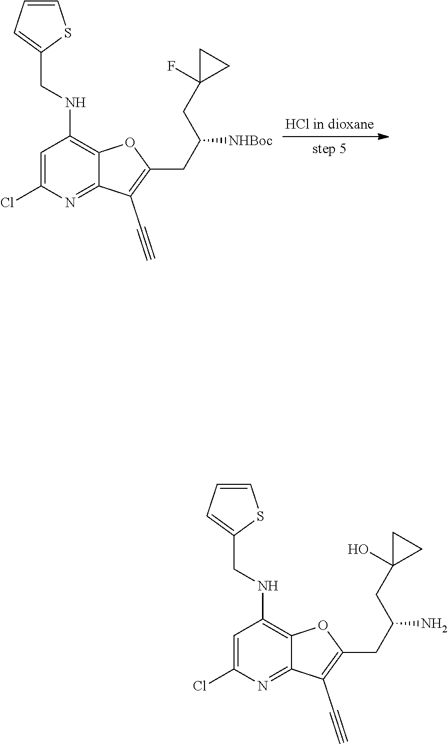 [SMILES: C#Cc1c(C[C@H](CC2(F)CC2)NC(=O)OC(C)(C)C)oc2c(NCc3cccs3)cc(Cl)nc12 ; C#Cc1c(C[C@@H](N)CC2(O)CC2)oc2c(NCc3cccs3)cc(Cl)nc12]

To a stirred solution tert-butyl N-[(2R)-1-{5-chloro-3-ethynyl-7-[(thiophen-2-ylmethyl)amino]furo[3,2-b]pyridin-2-yl}-3-(1-fluorocyclopropyl)propan-2-yl]carbamate (30 mg, 0.060 mmol, 1 equiv) in DCM (1 mL) was added HCl(gas) in 1,4-dioxane (1 mL, 4M) dropwise at 0° C. under air atmosphere. The resulting mixture was stirred for additional 2 h at room temperature. The resulting mixture was concentrated under reduced pressure. The residue was dissolved in MeOH (1 mL). The crude product (mg) was purified by Prep-HPLC with the following conditions (Column: XBridge Shield RP18 OBD Column 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 43% B to 63% B in 10 min; Wave Length: 254 nm/220 nm nm; RT1 (min): 10.0/11.62) to afford 1-[(2S)-2-amino-3-{5-chloro-3-ethynyl-7-[(thiophen-2-ylmethyl)amino]furo[3,2-b]pyridin-2-yl}propyl]cyclopropan-1-ol (6 mg, 25.08%).

LC-MS (ES, m/z): [M+H]⁺=402

¹H NMR (400 MHz, Methanol-d₄) δ 7.62 (s, 1H), 7.31 (dd, J=5.1, 1.2 Hz, 1H), 7.11 (dt, J=3.5, 1.1 Hz, 1H), 6.99 (dd, J=5.1, 3.5 Hz, 1H), 6.75 (s, 1H), 4.80 (s, 2H), 3.43 (s, 1H), 3.37 (s, 1H), 3.02 (s, 2H), 1.66 (d, J=8.2 Hz, 1H), 1.40 (dd, J=14.7, 7.3 Hz, 1H), 1.12 (d, J=6.9 Hz, 1H), 1.07 (d, J=7.1 Hz, 1H), 0.93 (t, J=7.3 Hz, 1H), 0.84-0.81 (m, 1H).

### Example 19: Synthesis of 3-bromo-5-chloro-2-[(2R)-2-(methylamino)pent-3-yn-1-yl]-N-(thiophen-2-ylmethyl)furo[3,2-b]pyridin-7-amine (Compound 295)

### Step 1. Synthesis of tert-butyl N-[(2R)-1-{3-bromo-5-chloro-7-iodofuro[3,2-b]pyridin-2-yl}pent-3-yn-2-yl]-N-methylcarbamate

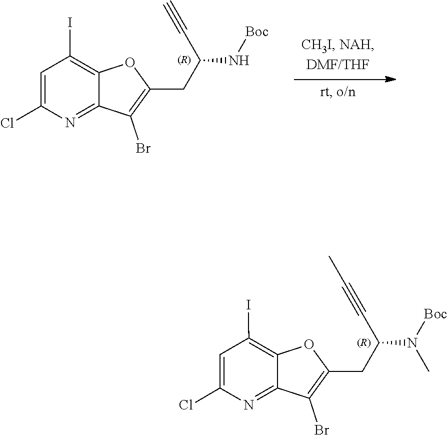

A solution of tert-butyl N-[(2R)-1-{3-bromo-5-chloro-7-iodofuro[3,2-b]pyridin-2-yl}but-3-yn-2-yl]carbamate (50 mg, 0.095 mmol, 1 equiv) in DMF/THF (1/3, 4 mL) was treated with NaH (60%) (2.97 mg, 0.124 mmol, 1.3 equiv) for 30 min at 0° C. under nitrogen atmosphere. To the above mixture was added CH₃I (14.85 mg, 0.105 mmol, 1.1 equiv) dropwise over 1 min at 0° C. The resulting mixture was stirred for additional overnight at room temperature. The residue was purified by silica gel column chromatography, eluted with PE/EA (6:1) to afford tert-butyl N-[(2R)-1-{3-bromo-5-chloro-7-iodofuro[3,2-b]pyridin-2-yl}pent-3-yn-2-yl]-N-methylcarbamate (crude) as a yellow solid.

### Step 2. Synthesis of tert-butyl N-[(2R)-1-{3-bromo-5-chloro-7-[(thiophen-2-ylmethyl)amino]furo[3,2-b]pyridin-2-yl}pent-3-yn-2-yl]-N-methylcarbamate

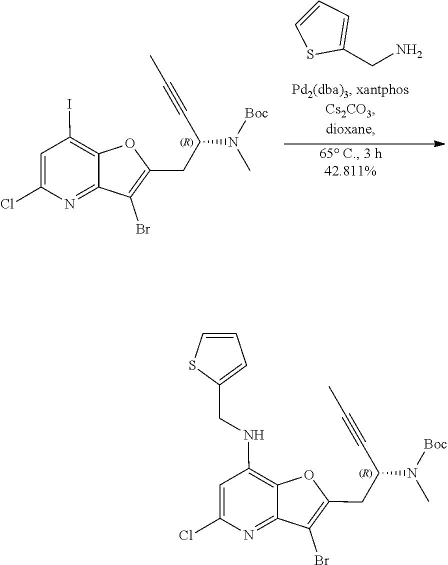

A mixture of tert-butyl N-[(2R)-1-{3-bromo-5-chloro-7-iodofuro[3,2-b]pyridin-2-yl}pent-3-yn-2-yl]-N-methylcarbamate (60 mg, 0.108 mmol, 1 equiv), 1-(thiophen-2-yl)methanamine (24.53 mg, 0.216 mmol, 2 equiv), Pd₂(dba)₃ (19.85 mg, 0.022 mmol, 0.2 equiv), xantphos (6.27 mg, 0.011 mmol, 0.1 equiv) and Cs₂CO₃ (70.62 mg, 0.216 mmol, 2 equiv) in dioxane (6 mL) was stirred for 3 h at 65° C. under nitrogen atmosphere. The residue was purified by silica gel column chromatography, eluted with PE/EA (1:1) to afford tert-butyl N-[(2R)-1-{3-bromo-5-chloro-7-[(thiophen-2-ylmethyl)amino]furo[3,2-b]pyridin-2-yl}pent-3-yn-2-yl]-N-methylcarbamate (25 mg, 42.81%) as a yellow solid.

### Step 3. Synthesis of 3-bromo-5-chloro-2-[(2R)-2-(methylamino)pent-3-yn-1-yl]-N-(thiophen-2-ylmethyl)furo[3,2-b]pyridin-7-amine

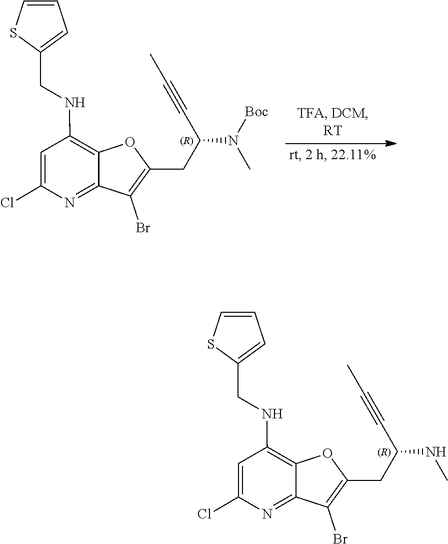

A solution of tert-butyl N-[(2R)-1-{3-bromo-5-chloro-7-[(thiophen-2-ylmethyl)amino]furo[3,2-b]pyridin-2-yl}pent-3-yn-2-yl]-N-methylcarbamate (25 mg, 0.046 mmol, 1 equiv) in DCM (2 mL) was treated with TFA (1 mL) for 1 min at room temperature. The resulting mixture was stirred for additional 2 h at room temperature. The reaction was monitored by LCMS. The crude product (90 mg) was purified by Prep-HPLC with the following conditions (Column: XBridge Prep C18 OBD Column, 19*150 mm, 5 μm; Mobile Phase A: Water (10 mmol/L NH₄HCO₃), Mobile Phase B: ACN; Flow rate: 25 mL/min; Gradient: 10% B to 35% B in 7 min, 35% B; Wave Length: 254/220 nm; RT1 (min): 6.35) to afford 3-bromo-5-chloro-2-[(2R)-2-(methylamino)pent-3-yn-1-yl]-N-(thiophen-2-ylmethyl)furo[3,2-b]pyridin-7-amine (4.5 mg, 22.11%).

LCMS [M+H]⁺=439

¹H NMR (400 MHz, Methanol-d₄) δ 7.30 (dd, J=5.1, 1.2 Hz, 1H), 7.07 (dq, J=3.3, 1.0 Hz, 1H), 6.97 (dd, J=5.1, 3.5 Hz, 1H), 6.58 (s, 1H), 3.76 (dd, J=8.9, 2.2 Hz, 1H), 3.44 (dq, J=8.7, 7.0 Hz, 1H), 2.41 (s, 3H), 1.49 (d, J=7.0 Hz, 3H).

### Example 20: Synthesis of 2-[(2S)-2-aminopropyl]-5-chloro-3-(1,3-oxazol-4-yl)-N-(thiophen-2-ylmethyl)furo[3,2-b]pyridin-7-amine (Compound 310)

### Step 1. Synthesis of tert-butyl N-{2-[(2S)-2-[(tert-butoxycarbonyl)amino]propyl]-5-chloro-3-(1,3-oxazol-5-yl)furo[3,2-b]pyridin-7-yl}-N-(thiophen-2-ylmethyl)carbamate

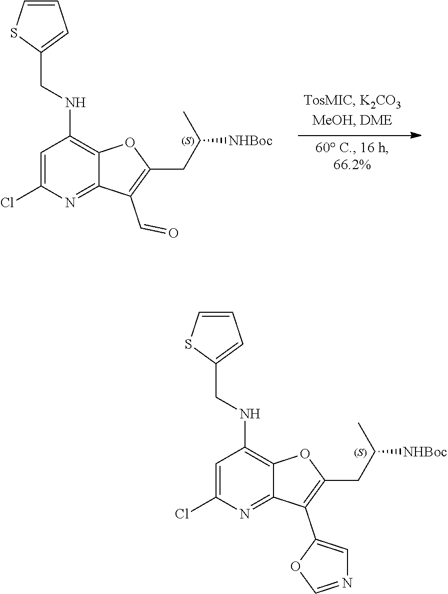

To a solution of tert-butyl N-[(2S)-1-{5-chloro-3-formyl-7-[(thiophen-2-ylmethyl)amino]furo[3,2-b]pyridin-2-yl}propan-2-yl]carbamate (150 mg, 0.333 mmol, 1 equiv) and TosMIC (71.60 mg, 0.366 mmol, 1.1 equiv) in MeOH (3 mL) and DME (0.9 mL) was added K₂CO₃ (92.15 mg, 0.666 mmol, 2 equiv). The resulting mixture was stirred for additional 16 h at 60° C. The residue was purified by reversed-phase flash chromatography with the following conditions: (column, C18; mobile phase, MeCN in Water (0.1% TFA), 20% to 100% gradient in 10 min; detector, UV 254 nm.) to afford tert-butyl N-{2-[(2S)-2-[(tert-butoxycarbonyl)amino]propyl]-5-chloro-3-(1,3-oxazol-5-yl)furo[3,2-b]pyridin-7-yl}-N-(thiophen-2-ylmethyl)carbamate (130 mg, 66.20%) as a white solid.

### Step 2. Synthesis of 2-[(2S)-2-aminopropyl]-5-chloro-3-(1,3-oxazol-4-yl)-N-(thiophen-2-ylmethyl)furo[3,2-b]pyridin-7-amine

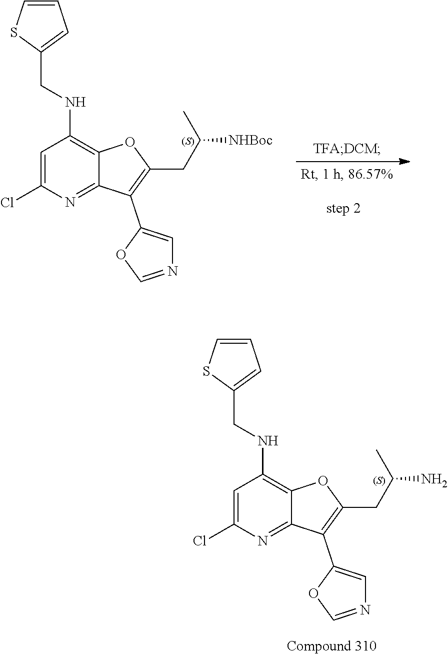

A solution of tert-butyl N-{2-[(2S)-2-[(tert-butoxycarbonyl)amino]propyl]-5-chloro-3-(1,3-oxazol-4-yl)furo[3,2-b]pyridin-7-yl}-N-(thiophen-2-ylmethyl)carbamate (70 mg, 0.119 mmol, 1 equiv) and TFA (0.5 mL) in DCM (1.5 mL) was stirred for 1 h at room temperature. The residue was purified by reversed-phase flash chromatography with the following conditions: (column, C₁₈; mobile phase, MeCN in Water (10 mmol/L NH₄HCO₃), 10% to 60% gradient in 10 min; detector, UV 254 nm.) to afford 2-[(2S)-2-aminopropyl]-5-chloro-3-(1,3-oxazol-4-yl)-N-(thiophen-2-ylmethyl)furo[3,2-b]pyridin-7-amine (40 mg, 86.57%).

LC-MS (ES, m/z): [M+H]⁺=388.8.

¹H NMR (400 MHz, Methanol-d₄) δ 8.30 (s, 1H), 7.74 (s, 1H), 7.30 (dd, J=5.1, 1.2 Hz, 1H), 7.12-7.05 (m, 1H), 6.98 (dd, J=5.1, 3.5 Hz, 1H), 6.59 (s, 1H), 4.77 (d, J=1.0 Hz, 2H), 3.45 (q, J=6.5 Hz, 1H), 3.20 (d, J=6.9 Hz, 2H), 1.17 (d, J=6.4 Hz, 3H).

### Example 21: Synthesis of 2-[(2S)-2-aminopropyl]-5-chloro-3-(1H-imidazol-2-yl)-N-(thiophen-2-ylmethyl)furo[3,2-b]pyridin-7-amine (Compound 313)

### Step 1. Synthesis of tert-butyl N-[(2S)-1-[5-chloro-3-(1H-imidazol-2-yl)-7-[(thiophen-2-ylmethyl)amino]furo[3,2-b]pyridin-2-yl]propan-2-yl]carbamate

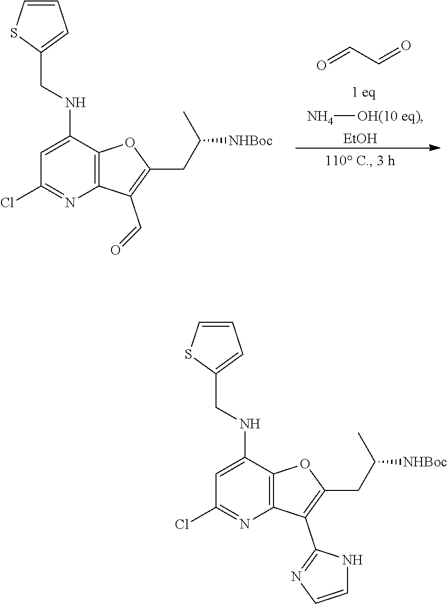

A solution of tert-butyl N-[(2S)-1-{5-chloro-3-formyl-7-[(thiophen-2-ylmethyl)amino]furo[3,2-b]pyridin-2-yl}propan-2-yl]carbamate (200 mg, 0.444 mmol, 1 equiv), NH₄₀H (155.78 mg, 4.440 mmol, 10 equiv) and glyoxal (25.80 mg, 0.444 mmol, 1 equiv) in EtOH (4 mL) was stirred for 3 h at 110° C. under nitrogen atmosphere. The residue was purified by reversed-phase flash chromatography with the following conditions: column, C₁₈; mobile phase, MeCN in Water (0.1% FA), 20% to 100% gradient in 15 min; detector, UV 254 nm. This resulted in tert-butyl N-[(2S)-1-[5-chloro-3-(1H-imidazol-2-yl)-7-[(thiophen-2-ylmethyl)amino]furo[3,2-b]pyridin-2-yl]propan-2-yl]carbamate (150 mg, 69.15%) as a red solid.

### Step 2. Synthesis of 2-[(2S)-2-aminopropyl]-5-chloro-3-(1H-imidazol-2-yl)-N-(thiophen-2-ylmethyl)furo[3,2-b]pyridin-7-amine

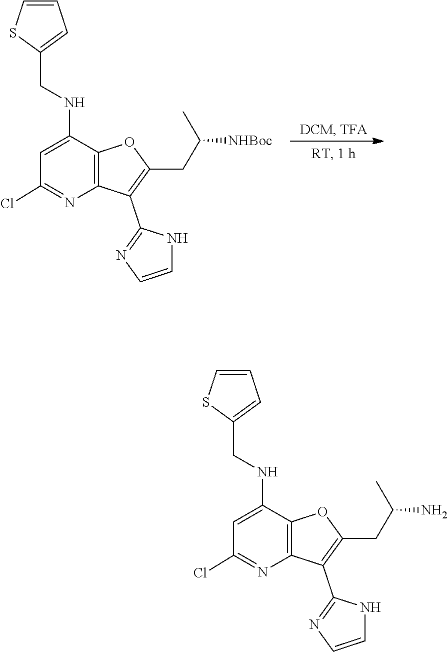 [SMILES: C[C@@H](Cc1oc2c(NCc3cccs3)cc(Cl)nc2c1-c1ncc[nH]1)NC(=O)OC(C)(C)C ; C[C@H](N)Cc1oc2c(NCc3cccs3)cc(Cl)nc2c1-c1ncc[nH]1]

A solution of tert-butyl N-[(2S)-1-[5-chloro-3-(1H-imidazol-2-yl)-7-[(thiophen-2-ylmethyl)amino]furo[3,2-b]pyridin-2-yl]propan-2-yl]carbamate (200 mg, 0.410 mmol, 1 equiv) and TFA (2 mL) in DCM (4 mL) was stirred for 1 h at room temperature under nitrogen atmosphere. The residue was purified by reversed-phase flash chromatography with the following conditions: column, C₁₈; mobile phase, MeCN in Water (0.1% FA), 20% to 100% gradient in 10 min; detector, UV 254 nm. This resulted in 2-[(2S)-2-aminopropyl]-5-chloro-3-(1H-imidazol-2-yl)-N-(thiophen-2-ylmethyl)furo[3,2-b]pyridin-7-amine (50 mg, 31.45%).

LC-MS (ES, m/z): [M+H]⁺=387.90

¹H NMR (400 MHz, Chloroform-d) δ 7.03 (d, J=3.4 Hz, 1H), 6.97 (dd, J=5.1, 3.4 Hz, 1H), 6.93 (s, 2H), 6.15 (s, 1H), 5.77 (s, 1H), 4.51 (t, J=5.5 Hz, 2H), 3.93 (s, 1H), 3.00 (s, 1H), 2.64 (s, 1H).

### Example 22: Synthesis of 3-bromo-5-chloro-2-(1-methoxyethyl)-N-(thiophen-2-ylmethyl)furo[3,2-b]pyridin-7-amine (Compound 356)

### Step 1. Synthesis of tert-butyl(3-bromo-5-chloro-2-(1-hydroxyethyl)furo[3,2-b]pyridin-7-yl)(thiophen-2-ylmethyl)carbamate

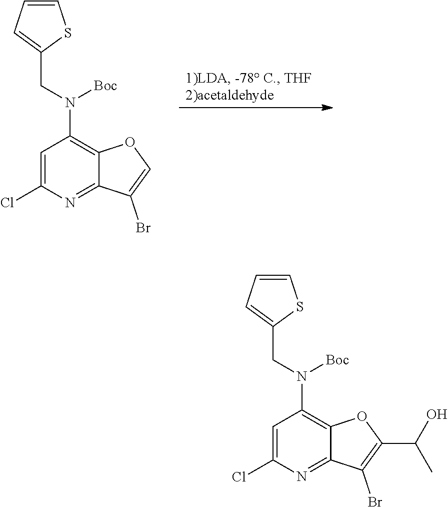 [SMILES: CC(C)(C)OC(=O)N(Cc1cccs1)c1cc(Cl)nc2c(Br)coc12 ; CC(O)c1oc2c(N(Cc3cccs3)C(=O)OC(C)(C)C)cc(Cl)nc2c1Br]

In a 100-mL round bottom flask, to a solution of tert-butyl N-{3-bromo-5-chlorofuro[3,2-b]pyridin-7-yl}-N-(thiophen-2-ylmethyl)carbamate (500 mg, 1.13 mmol, 1.0 equiv) in tetrahydrofuran (10 mL) was added dropwise lithium diisopropylamide (2 M in tetrahydrofuran/hexane, 0.62 mL, 1.24 mmol) at −78° C. under nitrogen atmosphere. The reaction mixture was stirred at −78° C. for 30 mins. Then a solution of acetaldehyde (83.5 mg, 1.13 mmol, 1.0 equiv) in 2 mL tetrahydrofuran was added dropwise and the mixture was stirred for another 60 mins. The reaction was quenched with aq. ammonium chloride (20 mL), and then the mixture was extracted with ethyl acetate (2×15 mL). The combined organic were washed with brine (10 mL), dried over anhydrous sodium sulphate, and concentrated under reduced pressure to yield a crude product which was directly purified by silica gel column chromatography, eluted with petroleum ether/ethyl acetate (5:1) to afford tert-butyl(3-bromo-5-chloro-2-(1-hydroxyethyl)furo[3,2-b]pyridin-7-yl)(thiophen-2-ylmethyl)carbamate (480 mg, 89.92%) as a white solid.

### Step 2. Synthesis of tert-butyl (3-bromo-5-chloro-2-(1-methoxyethyl) furo[3,2-b]pyridin-7-yl)(thiophen-2-ylmethyl)carbamate

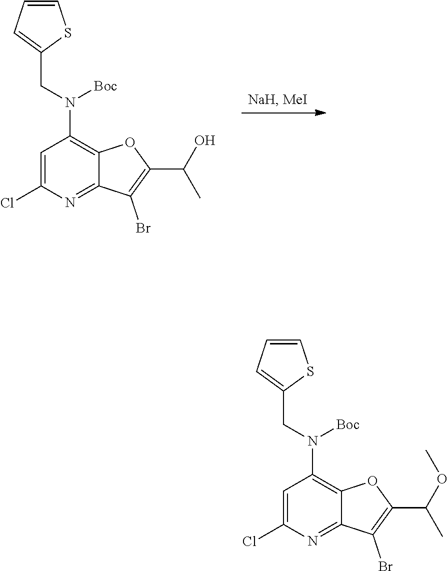 [SMILES: CC(O)c1oc2c(N(Cc3cccs3)C(=O)OC(C)(C)C)cc(Cl)nc2c1Br ; COC(C)c1oc2c(N(Cc3cccs3)C(=O)OC(C)(C)C)cc(Cl)nc2c1Br]

To a solution of tert-butyl(3-bromo-5-chloro-2-(1-hydroxyethyl)furo[3,2-b]pyridin-7-yl)(thiophen-2-yl-methyl)carbamate (300 mg, 0.69 mmol, 1 equiv) in N—N dimethylformamide (5 mL) was added sodium hydride (60% in oil, 49.2 mg) at 0° C. The mixture was stirred for 15 min. Iodomethane (96.0 mg, 0.68 mmol, 1.1 equiv) was added to the mixture and stirred for 2 h at room temperature. The reaction mixture was quenched by water and extracted with dichloromethane (3×20 mL). The combined organic layers were washed with brine (3×20 mL), dried over anhydrous sodium sulfate. After filtration, the filtrate was concentrated under reduced pressure. The crude product tert-butyl (3-bromo-5-chloro-2-(1-methoxyethyl) furo[3,2-b]pyridin-7-yl)(thiophen-2-ylmethyl)carbamate (120 mg, 38.88%) was used in the next step directly without further purification.

### Step 3. Synthesis of 3-bromo-5-chloro-2-(1-methoxyethyl)-N-(thiophen-2-ylmethyl)furo[3,2-b]pyridin-7-amine

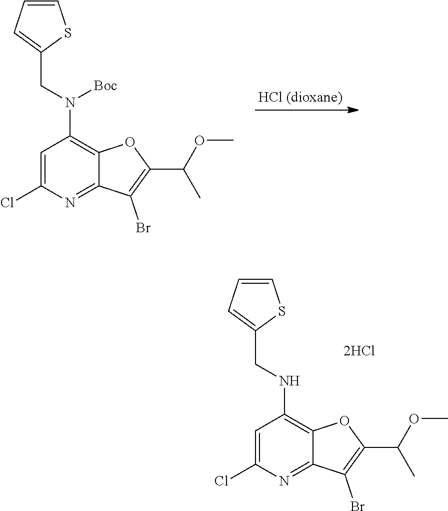 [SMILES: COC(C)c1oc2c(N(Cc3cccs3)C(=O)OC(C)(C)C)cc(Cl)nc2c1Br ; COC(C)c1oc2c(NCc3cccs3)cc(Cl)nc2c1Br]

A solution tert-butyl (3-bromo-5-chloro-2-(1-methoxyethyl)furo[3,2-b]pyridin-7-yl)(thiophen-2-yl-methyl)carbamate (100 mg, 0.225 mmol, 1 equiv) and HCl(4M) in dioxane (3 mL) was stirred for 1 h at room temperature. The resulting mixture was concentrated under reduced pressure. The residue was purified by trituration with ethyl ether (20 mL). The precipitated solids were collected by filtration and washed with ethyl ether (15 mL). This resulted in 3-bromo-5-chloro-2-(1-methoxyethyl)-N-(thiophen-2-ylmethyl)furo[3,2-b]pyridin-7-amine (22.4 mg, 27.98%).

LCMS (ES, m/z): [M+1]⁺=401, 403

¹HNMR (300 MHz, DMSO-d₆): δ 8.11 (s, 1H), 7.41 (dd, J=5.0, 1.3 Hz, 1H), 7.14-7.08 (m, 1H), 6.98 (dd, J=5.1, 3.4 Hz, 1H), 6.67 (s, 1H), 4.80-4.56 (m, 3H), 1.52 (d, J=6.6 Hz, 3H).

### Example 23: Synthesis of 1-(3-bromo-5-chloro-7-((thiophen-2-ylmethyl)amino)furo[3,2-b]pyridin-2-yl)ethan-1-ol (Compound 355)

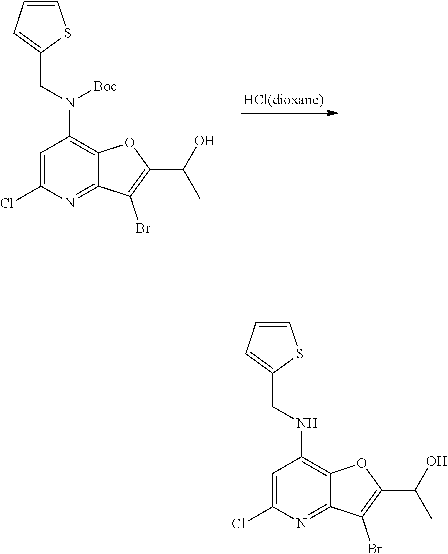 [SMILES: CC(O)c1oc2c(N(Cc3cccs3)C(=O)OC(C)(C)C)cc(Cl)nc2c1Br ; CC(O)c1oc2c(NCc3cccs3)cc(Cl)nc2c1Br]

A solution tert-butyl (3-bromo-5-chloro-2-(1-hydroxyethyl)furo[3,2-b]pyridin-7-yl)(thiophen-2-yl methyl)carbamate (100 mg, 0.23 mmol, 1.0 equiv) and HCl(4M) in dioxane (3 mL) was stirred for 1 h at room temperature. The resulting mixture was concentrated under reduced pressure. The residue was purified by trituration with ethyl ether (20 mL). The precipitated solids were collected by filtration and washed with ethyl ether (15 mL). This resulted in 1-(3-bromo-5-chloro-7-((thiophen-2-ylmethyl)amino)furo[3,2-b]pyridin-2-yl)ethan-1-ol (75.6 mg, 95.12%).

LCMS (ES, m/z): [M+1]⁺=387, 389

HNMR (300 MHz, DMSO-d₆): δ 8.04 (s, 1H), 7.40 (dd, J=5.0, 1.3 Hz, 1H), 7.11 (d, J=3.5 Hz, 1H), 6.98 (dd, J=5.1, 3.4 Hz, 1H), 6.64 (s, 1H), 4.73 (s, 2H), 1.48 (d, J=6.6 Hz, 3H).

### Example 24: Synthesis of 2-[(2S)-2-aminopropyl]-5-chloro-7-[(furan-2-ylmethyl)amino]-N,N-dimethylfuro[3,2-b]pyridine-3-carboxamide (Compound 357)

### Step 1. Synthesis of 2-[(2S)-2-[(tert-butoxycarbonyl)amino]propyl]-5-chloro-7-[(furan-2-ylmethyl)amino]furo[3,2-b]pyridine-3-carboxylic acid

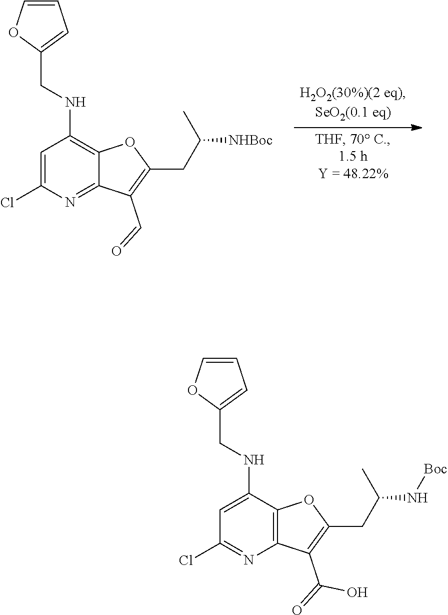 [SMILES: C[C@@H](Cc1oc2c(NCc3ccco3)cc(Cl)nc2c1C=O)NC(=O)OC(C)(C)C ; C[C@@H](Cc1oc2c(NCc3ccco3)cc(Cl)nc2c1C(=O)O)NC(=O)OC(C)(C)C]

A solution of tert-butyl N-[(2S)-1-{5-chloro-3-formyl-7-[(furan-2-ylmethyl)amino]furo[3,2-b]pyridin-2-yl}propan-2-yl]carbamate (600 mg, 1.383 mmol, 1 equiv), H₂O₂ (30%) (94.07 mg, 2.766 mmol, 2 equiv) and SeO2 (15.35 mg, 0.138 mmol, 0.1 equiv) in THE (10 mL) was stirred for 1.5 h at 70° C. under nitrogen atmosphere. This resulted in 2-[(2S)-2-[(tert-butoxycarbonyl)amino]propyl]-5-chloro-7-[(furan-2-ylmethyl)amino]furo[3,2-b]pyridine-3-carboxylic acid (300 mg, 48.22%) as a brown solid.

### Step 2. Synthesis of tert-butyl N-[(2S)-1-[5-chloro-3-(dimethylcarbamoyl)-7-[(furan-2-ylmethyl)amino]furo[3,2-b]pyridin-2-yl]propan-2-yl]carbamate

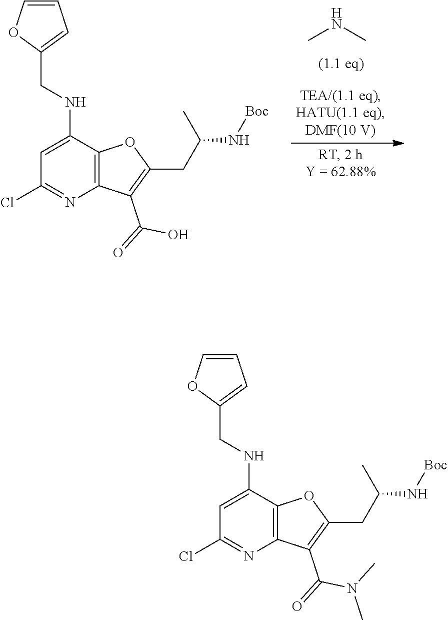

A solution of 2-[(2S)-2-[(tert-butoxycarbonyl)amino]propyl]-5-chloro-7-[(furan-2-ylmethyl)amino]furo[3,2-b]pyridine-3-carboxylic acid (300 mg, 0.667 mmol, 1 equiv) in DMF (2 mL) was treated with dimethylamine (33.07 mg, 0.734 mmol, 1.1 equiv), TEA (134.96 mg, 1.334 mmol, 2 equiv) for 2 min at room temperature under nitrogen atmosphere followed by the addition of HATU (278.91 mg, 0.734 mmol, 1.1 equiv) in portions at room temperature. The resulting mixture was stirred for 2 h at room temperature under nitrogen atmosphere. The residue was purified by reversed-phase flash chromatography with the following conditions: column, C₁₈; mobile phase, MeCN in Water (0.1% FA), 10% to 100% gradient in 20 min; detector, UV 254 nm. This resulted in tert-butyl N-[(2S)-1-[5-chloro-3-(dimethylcarbamoyl)-7-[(furan-2-ylmethyl)amino]furo[3,2-b]pyridin-2-yl]propan-2-yl]carbamate (200 mg, 62.88%) as a yellow solid.

### Step 3. Synthesis of 2-[(2S)-2-aminopropyl]-5-chloro-7-[(furan-2-ylmethyl)amino]-N,N-dimethylfuro[3,2-b]pyridine-3-carboxamide

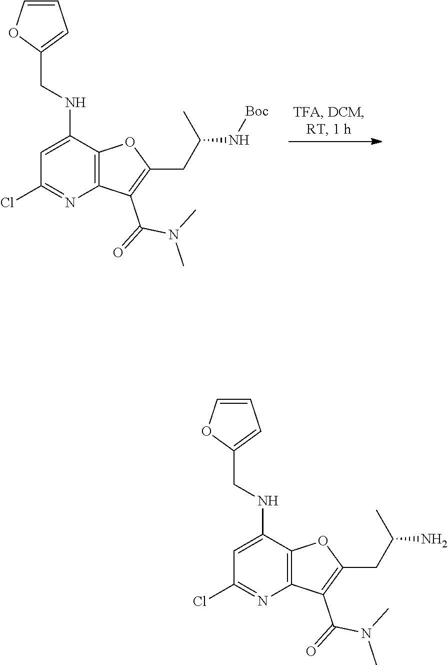 [SMILES: C[C@@H](Cc1oc2c(NCc3ccco3)cc(Cl)nc2c1C(=O)N(C)C)NC(=O)OC(C)(C)C ; C[C@H](N)Cc1oc2c(NCc3ccco3)cc(Cl)nc2c1C(=O)N(C)C]

A solution of tert-butyl N-[(2S)-1-[5-chloro-3-(dimethylcarbamoyl)-7-[(furan-2-ylmethyl)amino]furo[3,2-b]pyridin-2-yl]propan-2-yl]carbamate (200 mg, 0.419 mmol, 1 equiv) and TFA (1 mL) in DCM (2 mL) was stirred for 1 h at room temperature under nitrogen atmosphere. The residue was purified by reversed-phase flash chromatography with the following conditions: column, C₁₈; mobile phase, MeCN in Water (0.1% FA), 10% to 100% gradient in 20 min; detector, UV 254 nm. This resulted in 2-[(2S)-2-aminopropyl]-5-chloro-7-[(furan-2-ylmethyl)amino]-N,N-dimethylfuro[3,2-b]pyridine-3-carboxamide (30 mg, 18.99%).

LC-MS (ES, m/z): [M+H]⁺=376.90.

¹H NMR (400 MHz, Methanol-d₄) δ 8.52 (s, 1H), 7.45 (dd, J=1.9, 0.9 Hz, 1H), 6.68 (s, 1H), 6.40-6.32 (m, 2H), 4.57 (s, 2H), 3.73 (q, J=6.6 Hz, 1H), 3.17 (dd, J=6.4, 2.4 Hz, 2H), 3.14 (s, 3H), 3.05 (s, 3H), 1.38 (d, J=6.7 Hz, 3H).

### Example 25: Synthesis of 2-[(2S)-2-aminopropyl]-5-chloro-7-[(furan-2-ylmethyl)amino]-N-methylfuro[3,2-b]pyridine-3-carboxamide (Compound 358)

### Step 1. Synthesis of tert-butyl N-[(2S)-1-{5-chloro-7-[(furan-2-ylmethyl)amino]-3-(methylcarbamoyl)furo[3,2-b]pyridin-2-yl}propan-2-yl]carbamate

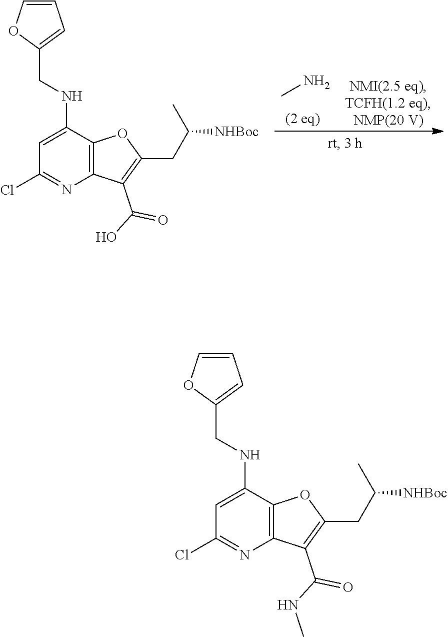

A solution of 2-[(2S)-2-[(tert-butoxycarbonyl)amino]propyl]-5-chloro-7-[(furan-2-ylmethyl)amino]furo[3,2-b]pyridine-3-carboxylic acid (45 mg, 0.100 mmol, 1 equiv) in NMP (1 mL) was treated with TCFH (33.68 mg, 0.120 mmol, 1.2 equiv) for 1 h at room temperature under nitrogen atmosphere followed by the addition of methylamine (6.21 mg, 0.200 mmol, 2 equiv) dropwise at room temperature. The resulting mixture was stirred for 3 h at room temperature under nitrogen atmosphere. The residue was purified by reversed-phase flash chromatography with the following conditions: column, C₁₈; mobile phase, MeCN in Water (0.1% FA), 10% to 100% gradient in 25 min; detector, UV 254 nm. This resulted in tert-butyl N-[(2S)-1-{5-chloro-7-[(furan-2-ylmethyl)amino]-3-(methylcarbamoyl)furo[3,2-b]pyridin-2-yl}propan-2-yl]carbamate (25 mg, 53.99%) as a white solid.

### Step 2. Synthesis of 2-[(2S)-2-aminopropyl]-5-chloro-7-[(furan-2-ylmethyl)amino]-N-methylfuro[3,2-b]pyridine-3-carboxamide

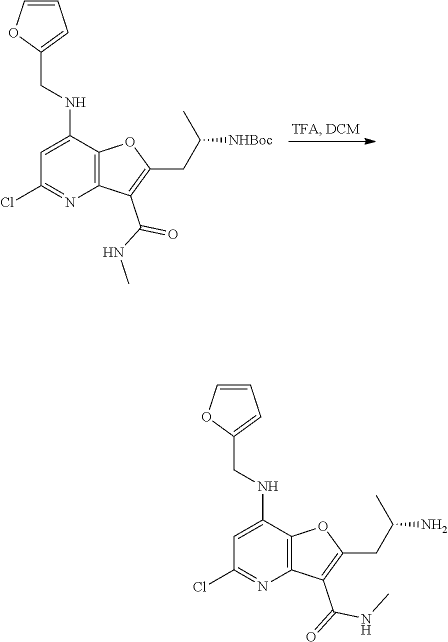 [SMILES: CNC(=O)c1c(C[C@H](C)NC(=O)OC(C)(C)C)oc2c(NCc3ccco3)cc(Cl)nc12 ; CNC(=O)c1c(C[C@H](C)N)oc2c(NCc3ccco3)cc(Cl)nc12]

A solution of tert-butyl N-[(2S)-1-{5-chloro-7-[(furan-2-ylmethyl)amino]-3-(methylcarbamoyl)furo[3,2-b]pyridin-2-yl}propan-2-yl]carbamate (50 mg, 0.108 mmol, 1 equiv) and TFA (0.5 mL) in DCM (1 mL) was stirred for 1 h at room temperature under nitrogen atmosphere. The residue was purified by reversed-phase flash chromatography with the following conditions: column, C₁₈; mobile phase, MeCN in Water (0.1% FA), 20% to 100% gradient in 25 min; detector, UV 254 nm. This resulted in 2-[(2S)-2-aminopropyl]-5-chloro-7-[(furan-2-ylmethyl)amino]-N-methylfuro[3,2-b]pyridine-3-carboxamide (20 mg, 51.04%).

LC-MS (ES, m/z): [M+H]⁺=362.85

¹H NMR (400 MHz, Methanol-d₄) δ 8.53 (s, 1H), 7.45 (d, J=1.8 Hz, 1H), 6.67 (s, 1H), 6.40-6.31 (m, 2H), 4.57 (s, 2H), 3.76 (p, J=6.6 Hz, 1H), 3.53 (dd, J=6.5, 1.6 Hz, 2H), 3.00 (s, 3H), 1.34 (d, J=6.7 Hz, 3H).

### Example 26: Synthesis of 3-bromo-5-chloro-2-(methoxymethyl)-N-(thiophen-2-ylmethyl)furo[3,2-b]pyridin-7-amine (Compound 360)

### Step 1. Synthesis of tert-butyl (3-bromo-5-chloro-2-(hydroxymethyl)furo[3,2-b]pyridin-7-yl)(thiophen-2-ylmethyl)carbamate

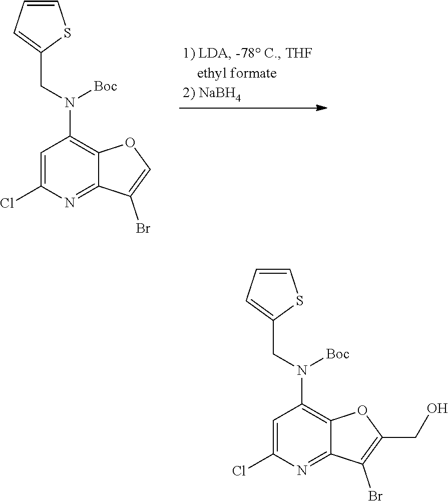 [SMILES: CC(C)(C)OC(=O)N(Cc1cccs1)c1cc(Cl)nc2c(Br)coc12 ; CC(C)(C)OC(=O)N(Cc1cccs1)c1cc(Cl)nc2c(Br)c(CO)oc12]

In a 100-mL round bottom flask, to a solution of tert-butyl N-{3-bromo-5-chlorofuro[3,2-b]pyridin-7-yl}-N-(thiophen-2-ylmethyl)carbamate (500 mg, 1.13 mmol, 1.0 equiv) in tetrahydrofuran (10 mL) was added dropwise lithium diisopropylamide (2 M in tetrahydrofuran/hexane, 0.62 mL, 1.24 mmol) at −78° C. under nitrogen atmosphere. The reaction mixture was stirred at −78° C. for 30 mins. Then a solution of ethyl formate (83.5 mg, 1.13 mmol, 1.0 equiv) in 2 mL tetrahydrofuran was added dropwise and the mixture was stirred for another 60 mins. The reaction was quenched with aq. ammonium chloride (20 mL), and then the mixture was extracted with ether/ethyl acetate (2×15 mL). The combined organic extracts were washed with brine (10 mL), dried over anhydrous sodium sulphate, and concentrated under reduced pressure to yield a crude product. Which was dissolved into 10 mL methanol, following by addition of NaBH₄ (85.5 mg, 2.26 mmol, 2.0 equiv) at 0° C. After stirring for 30 minutes, the reaction solution was concentrated and the crude was directly purified by silica gel column chromatography, eluted with petroleum ether/ethyl acetate (5:1) to afford tert-butyl (3-bromo-5-chloro-2-(hydroxymethyl)furo[3,2-b]pyridin-7-yl)(thiophen-2-ylmethyl)carbamate (480 mg, 89.92%) as a white solid.

### Step 2. Synthesis of 3-bromo-5-chloro-2-(methoxymethyl)-N-(thiophen-2-ylmethyl)furo[3,2-b]pyridin-7-amine

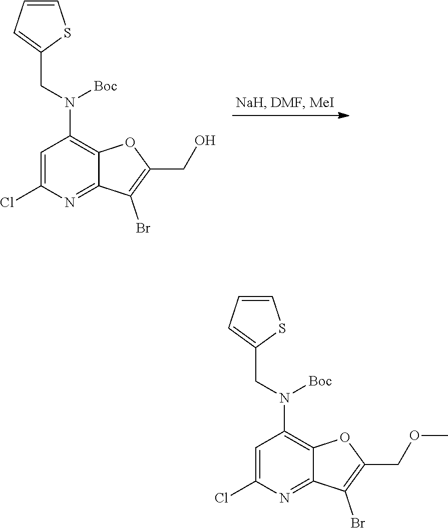 [SMILES: CC(C)(C)OC(=O)N(Cc1cccs1)c1cc(Cl)nc2c(Br)c(CO)oc12 ; COCc1oc2c(N(Cc3cccs3)C(=O)OC(C)(C)C)cc(Cl)nc2c1Br]

To a solution of tert-butyl (3-bromo-5-chloro-2-(hydroxymethyl)furo[3,2-b]pyridin-7-yl)(thiophen-2-ylmethyl)carbamate (200 mg, 0.69 mmol, 1 equiv) in N—N dimethylformamide (5 mL) was added sodium hydride (60% in oil, 49.2 mg) at 0° C. The mixture was stirred for 15 min. Iodomethane (96.0 mg, 0.68 mmol, 1.1 equiv) was added to the mixture and stirred for 2 h at room temperature. The reaction mixture was quenched by water and extracted with dichloromethane (3×20 mL). The combined organic layers were washed with brine (3×20 mL), dried over anhydrous sodium sulfate. After filtration, the filtrate was concentrated under reduced pressure. The crude product 3-bromo-5-chloro-2-(methoxymethyl)-N-(thiophen-2-ylmethyl)furo[3,2-b]pyridin-7-amine (130 mg, 38.88%) was used in the next step directly without further purification.

### Step 3. Synthesis of 3-bromo-5-chloro-2-(methoxymethyl)-N-(thiophen-2-ylmethyl)furo[3,2-b]pyridin-7-amine

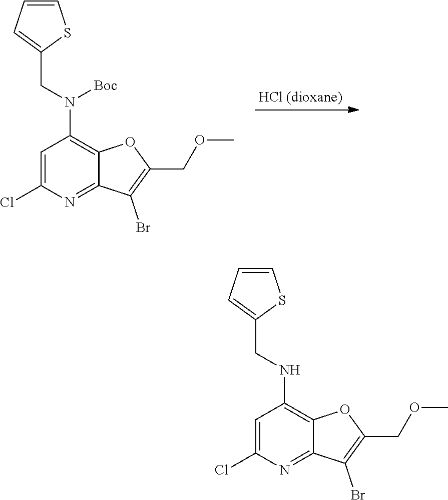 [SMILES: COCc1oc2c(N(Cc3cccs3)C(=O)OC(C)(C)C)cc(Cl)nc2c1Br ; COCc1oc2c(NCc3cccs3)cc(Cl)nc2c1Br]

A solution 3-bromo-5-chloro-2-(methoxymethyl)-N-(thiophen-2-ylmethyl)furo[3,2-b]pyridin-7-amine (100 mg, 0.23 mmol, 1.0 equiv) and HCl (4 M) in dioxane (3 mL) was stirred for 1 h at room temperature. The resulting mixture was concentrated under reduced pressure. The residue was purified by trituration with ethyl ether (20 mL). The precipitated solids were collected by filtration and washed with ethyl ether (15 mL). This resulted in 3-bromo-5-chloro-2-(methoxymethyl)-N-(thiophen-2-ylmethyl)furo[3,2-b]pyridin-7-amine HCl salt (13.8 mg, 17.36%).

LCMS (ES, m/z): [M+1]⁺=387, 389.

¹HNMR (300 MHz, DMSO-d₆): δ 8.17 (s, 1H), 7.39 (d, J=5.0 Hz, 1H), 7.10 (d, J=3.4 Hz, 1H), 6.97 (t, J=4.3 Hz, 1H), 6.67 (s, 1H), 4.71 (s, 2H), 4.58 (s, 2H), 4.29 (s, 3H).

### Example 27: Synthesis of rac-2-[(1R,2S)-2-aminocyclohexyl]-5-chloro-N-(thiophen-2-ylmethyl)furo[3,2-b]pyridin-7-amine (Compound 363)

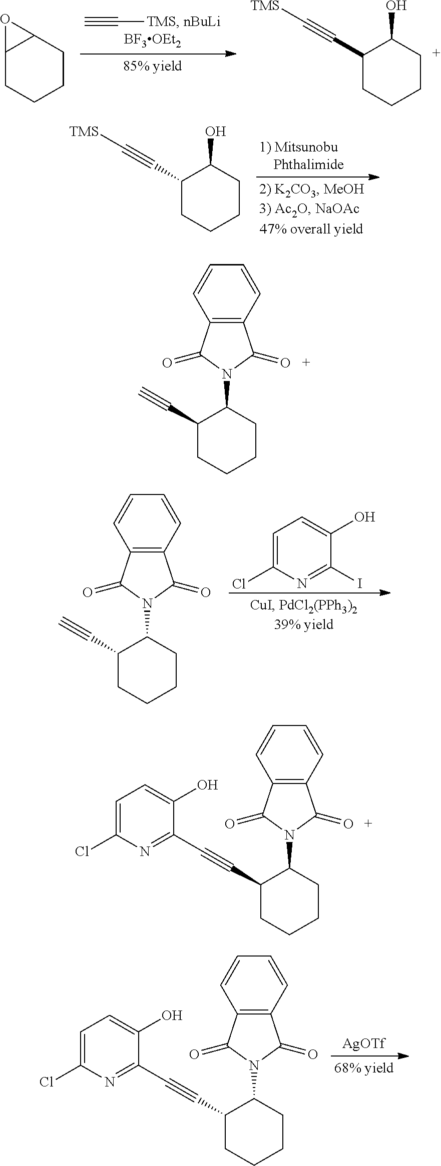

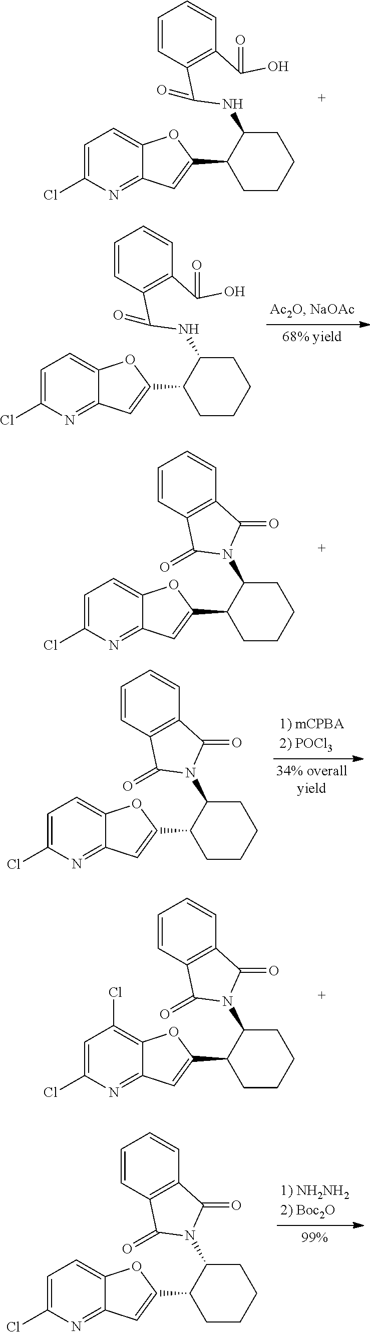

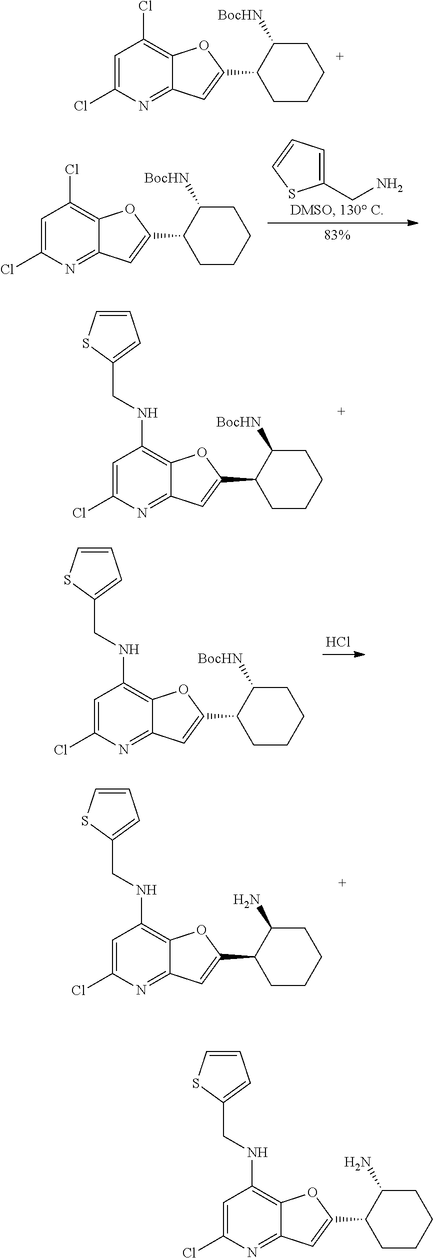

### Step 1, Synthesis of Rac-(1R,2S)-2-((trimethylsilyl)ethynyl)cyclohexan-1-ol

To a solution of ethynyltrimethylsilane (1.89 g, 2.80 mL, 1.5 Eq, 19.3 mmol) in THF (30.00 mL) at −70° C. was added n-BuLi (1.24 g, 7.72 mL, 2.50 molar, 1.5 Eq, 19.3 mmol) slowly. The reaction mixture was stirred for 10 minutes at −70° C. and BF₃·OEt₂ (2.91 g, 2.60 mL, 1.60 Eq, 20.5 mmol) was added. The reaction mixture was stirred for 10 minutes at −70° C. and cyclohexeneoxide (1.26 g, 1.30 mL, 1 Eq, 12.9 mmol) was added dropwise. The reaction mixture was stirred at −70° C. for 2 hours and quenched with a saturated solution of ammonium chloride (50 mL). The reaction mixture was extracted with DCM (2×75 mL). The combined organic layers were washed with brine, dried over Na₂SO₄, filtered and concentrated in vacuo. The crude material was purified by column chromatography (250 mL silica, Hept/EtOAc, 85/15 to afford rac-(1R,2S)-2-((trimethylsilyl)ethynyl)cyclohexan-1-ol (2.15 g, 10.9 mmol, 85.1% yield)

### Step 2. Synthesis of rac-2-((1R,2R)-2-((trimethylsilyl)ethynyl)cyclohexyl)isoindoline-1,3-dione

DIAD (14.1 g, 13.6 mL, 1.50 Eq, 69.9 mmol) was added dropwise to a solution of rac-(1R,2S)-2-((trimethylsilyl)ethynyl)cyclohexan-1-ol (9.16 g, 1.00 Eq, 46.6 mmol) and triphenylphosphine (18.4 g, 1.50 Eq, 70.2 mmol) in THE (200 mL) at 0° C. The resulting mixture was stirred for 30 min at 0° C. after which phthalimide (10.3 g, 1.50 Eq, 70.0 mmol) was added and stirred at room temperature for 16 hours. The reaction mixture was concentrated in vacuo and the crude material was purified by automated column chromatography. Fractions containing product were combined and concentrated in vacuo to afford rac-2-((1R,2R)-2-((trimethylsilyl)ethynyl)cyclohexyl)isoindoline-1,3-dione (8.88 g, 27.3 mmol, 58.5% yield) as a white solid.

### Step 3. Synthesis of rac-2-(((1R,2R)-2-ethynylcyclohexyl)carbamoyl)benzoic acid

To a solution of 2-((1R,2R)-2-((trimethylsilyl)ethynyl)cyclohexyl)isoindoline-1,3-dione (8.88 g, 1.00 Eq, 27.3 mmol) in MeOH (150 mL) was added potassium carbonate (5.66 g, 1.50 Eq, 40.9 mmol). The mixture was stirred at room temperature for 16 hours. The reaction mixture was concentrated after which water was added (100 mL). The aqueous layer was acidified with 37% HCl to pH 2 and extracted with EtOAc (3×100 mL). The combined organic layers were dried over Na₂SO₄, filtered and concentrated in vacuo to afford rac-2-(((1R,2R)-2-ethynylcyclohexyl)carbamoyl)benzoic acid (7.31 g, 26.9 mmol, 98.8% yield) as a white solid.

### Step 4. Synthesis of rac-2-((1S,2S)-2-ethynylcyclohexyl)isoindoline-1,3-dione

A mixture of rac-2-(((1R,2R)-2-ethynylcyclohexyl)carbamoyl)benzoic acid (7.31 g, 1.00 Eq, 26.9 mmol) and sodium acetate (2.21 g, 1.00 Eq, 26.9 mmol) in acetic anhydride (110 g, 102 mL, 40.1 Eq, 1.08 mol) was heated to reflux for 2 hours. The mixture was quenched with water (500 mL) and allowed to cool to room temperature. The mixture was extracted with DCM (3×200 mL), dried over Na₂SO₄, filtered and concentrated in vacuo. The crude material was purified by automated flash column chromatography to afford rac-2-((1S,2S)-2-ethynylcyclohexyl)isoindoline-1,3-dione (5.60 g, 22.1 mmol, 82.1% yield) was isolated as a white solid.

### Step 5. Synthesis of rac-2-((1S,2S)-2-((6-chloro-3-hydroxypyridin-2-yl)ethynyl)cyclohexyl)isoindoline-1,3-dione

A portion of rac-2-((1S,2S)-2-ethynylcyclohexyl)isoindoline-1,3-dione (5.60 g, 1.00 Eq, 22.1 mmol), 6-chloro-2-iodopyridin-3-ol (6.30 g, 1.12 Eq, 24.7 mmol) and triethylamine (18 g, 25 mL, 8.1 Eq, 0.18 mol) were dissolved in THE (25 mL). Copper(I) iodide (178 mg, 0.0423 Eq, 935 mol) and bis(triphenylphosphine)palladium(II) dichloride (326 mg, 0.0210 Eq, 464 mol) were added to the solution and the mixture was heated at 50° C. for 2 hours under a N₂ atmosphere. The mixture was concentrated in vacuo, redissolved in DCM, coated on silica and dried in vacuo. The crude material was purified by automated column chromatography. Fractions containing product were combined and concentrated in vacuo to afford rac-2-((1S,2S)-2-((6-chloro-3-hydroxypyridin-2-yl)ethynyl)cyclohexyl)isoindoline-1,3-dione (3.27 g, 8.59 mmol, 38.8% yield) as an off-white solid.

### Step 6. Synthesis of rac-2-(((1S,2R)-2-(5-chlorofuro[3,2-b]pyridin-2-yl)cyclohexyl)carbamoyl)benzoic acid

Solid rac-2-((1S,2S)-2-((6-chloro-3-hydroxypyridin-2-yl)ethynyl)cyclohexyl)isoindoline-1,3-dione (3.27 g, 60.7% Wt, 1.00 Eq, 5.21 mmol) was suspended in MeOH (25 mL). Potassium carbonate (2.16 g, 3.00 Eq, 15.6 mmol) and silver trifluoromethanesulfonate (402 mg, 0.300 Eq, 1.56 mmol), no lights, were added and the reaction mixture was heated at 50° C. for 8 hours. The reaction mixture was filtered over Celite, washed with MeOH and the combined organic layers were concentrated in vacuo. The crude material was purified by automated column chromatography. Fractions containing product were combined and concentrated in vacuo to afford rac-2-(((1S,2R)-2-(5-chlorofuro[3,2-b]pyridin-2-yl)cyclohexyl)carbamoyl)benzoic acid (1.41 g, 3.54 mmol, 67.8% yield) as an off-white solid.

### Step 7. Synthesis of rac-2-((1S,2R)-2-(5-chlorofuro[3,2-b]pyridin-2-yl)cyclohexyl)isoindoline-1,3-dione

A mixture of rac-2-(((1S,2R)-2-(5-chlorofuro[3,2-b]pyridin-2-yl)cyclohexyl)carbamoyl)benzoic acid (1.41 g, 1.00 Eq, 3.54 mmol) and sodium acetate (300 mg, 1.03 Eq, 3.66 mmol) in acetic anhydride (16 g, 15 mL, 45 Eq, 0.16 mol) was heated to reflux and stirred for 2 hours. The mixture was quenched with water and allowed to cool to room temperature after which it was extracted with DCM (3×25 mL), dried over Na₂SO₄, filtered and concentrated in vacuo. The crude material was purified by automated column chromatography. Fractions containing product were combined and concentrated in vacuo to provide Synthesis of rac-2-((1S,2R)-2-(5-chlorofuro[3,2-b]pyridin-2-yl)cyclohexyl)isoindoline-1,3-dione (1.028 g, 2.699 mmol, 76.4% yield) as a white solid.

### Step 8. Synthesis of rac-5-Chloro-2-((1R,2S)-2-(1,3-dioxoisoindolin-2-yl)cyclohexyl)furo[3,2-b]pyridine 4-oxide

To a solution of rac-2-((1S,2R)-2-(5-chlorofuro[3,2-b]pyridin-2-yl)cyclohexyl)isoindoline-1,3-dione (1.318 g, 1.00 Eq, 3.461 mmol) in DCM (15 mL) was added mCPBA (1.42 g, 77% Wt, 1.83 Eq, 6.34 mmol). The reaction mixture was stirred at room temperature for 16 hours. The mixture was poured in a sat. NaHCO₃ solution (100 mL) and extracted with DCM (3×50 mL). The combined organic layers were washed with brine (100 mL), dried over Na₂SO₄, filtered and concentrated in vacuo. The crude material was purified by automated column chromatography. Fractions containing product were combined and concentrated in vacuo to afford rac-5-Chloro-2-((1R,2S)-2-(1,3-dioxoisoindolin-2-yl)cyclohexyl)furo[3,2-b]pyridine 4-oxide (1.04 g, 2.62 mmol, 75.7% yield) as a pale yellow solid/foam.

### Step 9. Synthesis of rac-2-((1S,2R)-2-(5,7-dichlorofuro[3,2-b]pyridin-2-yl)cyclohexyl)isoindoline-1,3-dione

To a solution of rac-5-chloro-2-((1R,2S)-2-(1,3-dioxoisoindolin-2-yl)cyclohexyl)furo[3,2-b]pyridine 4-oxide (1.04 g, 1.00 Eq, 2.62 mmol) in chloroform (15 mL) was added phosphoryl chloride (7.24 g, 4.40 mL, 18.0 Eq, 47.2 mmol). The reaction mixture was stirred at reflux for 3.5 hours. The reaction mixture was concentrated in vacuo after which the residue was dissolved in DCM (25 mL). A saturated NaHCO₃ solution (50 mL) was added and the mixture was extracted with DCM (3×25 mL). The combined organic layers were washed with brine (50 mL), dried over Na₂SO₄, filtered and concentrated in vacuo. The crude material was purified by automated column chromatography. Fractions containing product were combined and concentrated in vacuo to afford rac-2-((1S,2R)-2-(5,7-dichlorofuro[3,2-b]pyridin-2-yl)cyclohexyl)isoindoline-1,3-dione (494 mg, 1.19 mmol, 45.4% yield) as a white solid.

### Step 10. Synthesis of rac-tert-Butyl ((1S,2R)-2-(5,7-dichlorofuro[3,2-b]pyridin-2-yl)cyclohexyl)carbamate

A portion of 2-((1S,2R)-2-(5,7-dichlorofuro[3,2-b]pyridin-2-yl)cyclohexyl)isoindoline-1,3-dione (100 mg, 1 Eq, 241 mol) was dissolved in ethanol (3.0 mL) and hydrazine hydrate (37.1 mg, 35.9 μL, 65% Wt, 2 Eq, 482 mol) was added. Then, mixture was refluxed, and reaction progress was monitored by LC-MS. After 2 hours, the mixture was diluted with 2 mL of water and extracted with 2×20 mL of EtOAc. The combined extracts were dried over Na₂SO₄ and concentrated to afford 90 mg of a red oil which was immediately dissolved in 3 mL of EtOAc. To this was added Boc₂O (184 mg, 194 μL, 3.5 Eq, 843 mol) and mixture was stirred overnight. Subsequently, the mixture was diluted with 10 mL of EtOAc and washed twice with 4 mL of 1N HCl. The organic layer was then dried over Na₂SO₄ and concentrated to afford rac-tert-butyl ((1S,2R)-2-(5,7-dichlorofuro[3,2-b]pyridin-2-yl)cyclohexyl)carbamate (90 mg, 0.23 mmol, 97%) as colorless oil.

### Step 11. Synthesis of rac-tert-Butyl ((1S,2R)-2-(5-chloro-7-((thiophen-2-ylmethyl)amino)furo[3,2-b]pyridin-2-yl)cyclohexyl)carbamate (32)

A portion of tert-butyl ((1S,2R)-2-(5,7-dichlorofuro[3,2-b]pyridin-2-yl)cyclohexyl)carbamate (20 mg, 1 Eq, 52 mol) was dissolved in DMSO (1.0 mL) and thiophen-2-ylmethanamine (59 mg, 53 μL, 10 Eq, 0.52 mmol) was added. The mixture was stirred at 130° C. overnight. The mixture was allowed to cool down to rt, diluted with 10 mL of EtOAc and washed with 3×2 mL of water. The organic layer was dried over Na₂SO₄ and concentrated to afford 200 mg of an orange liquid. This was purified on silica to afford rac-tert-butyl ((1S,2R)-2-(5-chloro-7-((thiophen-2-ylmethyl)amino)furo[3,2-b]pyridin-2-yl)cyclohexyl)carbamate (20 mg, 43 mol, 83%) as brown solid.

### Step 12. Synthesis of rac-2-[(1R,2S)-2-aminocyclohexyl]-5-chloro-N-(thiophen-2-ylmethyl)furo[3,2-b]pyridin-7-amine

To a stirred solution of rac-tert-butyl N-{2-[(1R,2S)-2-[(tert-butoxycarbonyl)amino]cyclohexyl]-5-chlorofuro[3,2-b]pyridin-7-yl}-N-(thiophen-2-ylmethyl)carbamate (42.4 mg, 0.075 mmol, 1 equiv) in DCM (0.5 mL) was added HCl(gas) in 1,4-dioxane (1 mL) dropwise at 0° C. The resulting mixture was stirred for 1 h at room temperature. The mixture was basified to pH 8 with DIEA at 0° C. The resulting mixture was concentrated under vacuum. The residue was purified by reversed-phase flash chromatography with the following conditions: column, C₁₈; mobile phase, MeCN in Water (10 mmol/L NH₄HCO₃), 0% to 100% gradient in 20 min; detector, UV 254 nm. This resulted in rac-2-[(1R,2S)-2-aminocyclohexyl]-5-chloro-N-(thiophen-2-ylmethyl)furo[3,2-b]pyridin-7-amine (11.8 mg, 41.89%).

LC-MS (ES, m/z): [M+H]⁺=362.

¹H-NMR (400 MHz, Methanol-d₄) δ 7.34-7.24 (m, 1H), 7.12-7.02 (m, 1H), 7.02-6.92 (m, 1H), 6.57-6.43 (m, 2H), 4.72 (s, 2H), 3.52-3.42 (m, 1H), 3.22-3.12 (m, 1H), 2.01-1.42 (m, 8H).

### Example 28: Synthesis of 2-[(2S)-2-aminopropyl]-5-chloro-N-(furan-2-ylmethyl)-3-(1,3,4-oxadiazol-2-yl)furo[3,2-b]pyridin-7-amine (Compound 364)

### Step 1. Synthesis of tert-butyl N-[(2S)-1-{5-chloro-7-[(furan-2-ylmethyl)amino]-3-(1,3,4-oxadiazol-2-yl) furo[3,2-b]pyridin-2-yl}propan-2-yl]carbamate

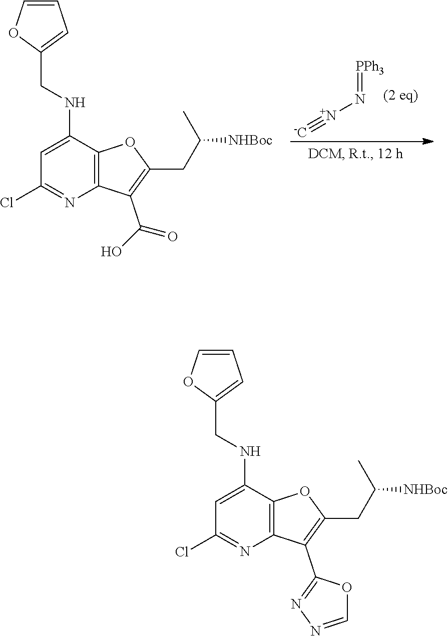

A solution of 2-[(2S)-2-[(tert-butoxycarbonyl)amino]propyl]-5-chloro-7-[(furan-2-ylmethyl)amino]furo[3,2-b]pyridine-3-carboxylic acid (300 mg, 0.667 mmol, 1 equiv) and isocyano(triphenyl-lambda5-phosphanylidene)amine (201.59 mg, 0.667 mmol, 1 equiv) in DCM (5 mL) was stirred for 12 h at room temperature under nitrogen atmosphere. The residue was purified by reversed-phase flash chromatography with the following conditions: column, C₁₈; mobile phase, MeCN in Water (0.1% FA), 10% to 100% gradient in 20 min; detector, UV 254 nm. This resulted in tert-butyl N-[(2S)-1-{5-chloro-7-[(furan-2-ylmethyl)amino]-3-(1,3,4-oxadiazol-2-yl) furo[3,2-b]pyridin-2-yl}propan-2-yl]carbamate (50 mg, 15.82%) as a yellow solid.

### Step 2. Synthesis of 2-[(2S)-2-aminopropyl]-5-chloro-N-(furan-2-ylmethyl)-3-(1,3,4-oxadiazol-2-yl)furo[3,2-b]pyridin-7-amine

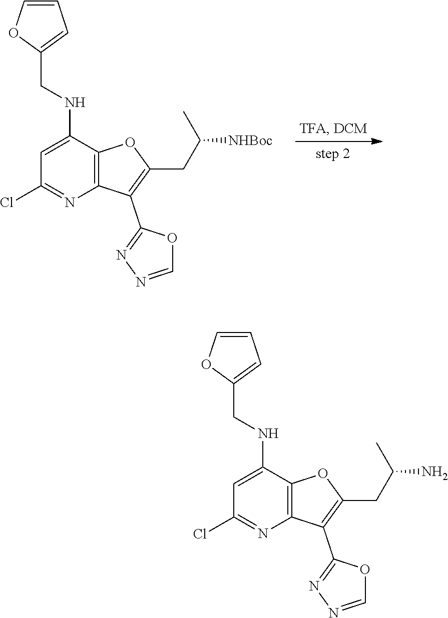 [SMILES: C[C@@H](Cc1oc2c(NCc3ccco3)cc(Cl)nc2c1-c1nnco1)NC(=O)OC(C)(C)C ; C[C@H](N)Cc1oc2c(NCc3ccco3)cc(Cl)nc2c1-c1nnco1]

A solution of tert-butyl N-[(2S)-1-{5-chloro-7-[(furan-2-ylmethyl)amino]-3-(1,3,4-oxadiazol-2-yl)furo[3,2-b]pyridin-2-yl}propan-2-yl]carbamate (50 mg, 0.106 mmol, 1 equiv) and TFA (0.5 mL) in DCM (1 mL) was stirred for 1 h at room temperature under nitrogen atmosphere. The residue was purified by reversed-phase flash chromatography with the following conditions: column, C₁₈; mobile phase, MeCN in Water (10 mmol/L NH₄HCO₃), 10% to 100% gradient in 20 min; detector, UV 254 nm. This resulted in 2-[(2S)-2-aminopropyl]-5-chloro-N-(furan-2-ylmethyl)-3-(1,3,4-oxadiazol-2-yl)furo[3,2-b]pyridin-7-amine (15 mg, 38.03%).

LC-MS [M+H]⁺=374.05.

¹H NMR (400 MHz, Methanol-d₄) δ 9.10 (s, 1H), 8.53 (s, 1H), 7.46 (dd, J=1.8, 0.9 Hz, 1H), 6.71 (s, 1H), 6.41-6.34 (m, 2H), 4.58 (s, 3H), 3.81 (q, J=6.6 Hz, 1H), 3.60-3.41 (m, 2H), 1.36 (d, J=6.6 Hz, 3H).

### Example 29: Synthesis of 2-{2-[(2S)-2-aminopropyl]-5-chloro-7-[(furan-2-ylmethyl)amino]furo[3,2-b]pyridin-3-yl}-1,3-oxazole-5-carboxamide (Compound 365)

### Step 1. Synthesis of tert-butyl N-[(2S)-1-{5-chloro-7-[(furan-2-ylmethyl)amino]-3-[(prop-2-yn-1-yl)carbamoyl]furo[3,2-b]pyridin-2-yl}propan-2-yl]carbamate

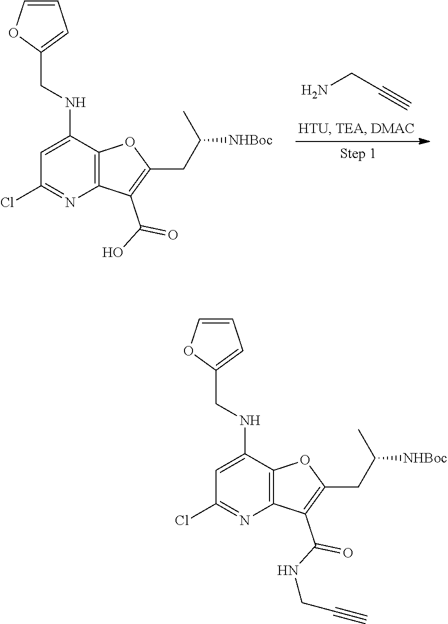

To a stirred mixture of 2-[(2S)-2-[(tert-butoxycarbonyl)amino]propyl]-5-chloro-7-[(furan-2-ylmethyl) amino]furo[3,2-b]pyridine-3-carboxylic acid (600 mg, 1.334 mmol, 1 equiv), TEA (0.27 g, 2.668 mmol, 2 equiv) and 2-propynylamine (0.08 g, 1.467 mmol, 1.1 equiv) in DMAC (10 mL) was added HATU (0.56 g, 1.467 mmol, 1.1 equiv) in portions at room temperature under nitrogen atmosphere. The resulting mixture was stirred for 3 h at room temperature under nitrogen atmosphere. The residue was purified by reversed-phase flash chromatography with the following conditions: column, C₁₈; mobile phase, MeCN in Water (0.1% FA), 10% to 100% gradient in 20 min; detector, UV 254 nm. This resulted in tert-butyl N-[(2S)-1-{5-chloro-7-[(furan-2-ylmethyl)amino]-3-[(prop-2-yn-1-yl)carbamoyl]furo[3,2-b]pyridin-2-yl}propan-2-yl]carbamate (400 mg, 61.59%) as a yellow solid.

### Step 2. Synthesis of tert-butyl N-[(2S)-1-[3-(4-carbamoyl-1,3-oxazol-2-yl)-5-chloro-7-[(furan-2-ylmethyl) amino]furo[3,2-b]pyridin-2-yl]propan-2-yl]carbamate

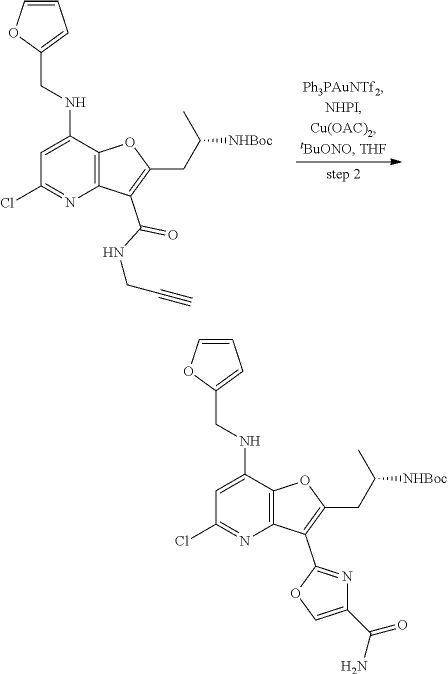

To a stirred mixture of tert-butyl N-[(2S)-1-{5-chloro-7-[(furan-2-ylmethyl)amino]-3-[(prop-2-yn-1-yl) carbamoyl]furo[3,2-b]pyridin-2-yl}propan-2-yl]carbamate (150 mg, 0.308 mmol, 1 equiv), NHPI (15.08 mg, 0.092 mmol, 0.3 equiv), Cu(OAC)₂ (8.39 mg, 0.046 mmol, 0.15 equiv) and (Ph₃P)AuNTf₂ (7.59 mg, 0.010 mmol, 0.1 equiv) in THE (5 mL) was added tBuONO (95.30 mg, 0.924 mmol, 3 equiv) dropwise at room temperature under nitrogen atmosphere. The resulting mixture was stirred for 12 h at 80° C. under nitrogen atmosphere. The residue was purified by reversed-phase flash chromatography with the following conditions: column, C18; mobile phase, MeCN in Water (0.1% FA), 10% to 100% gradient in 20 min; detector, UV 254 nm. This resulted in tert-butyl N-[(2S)-1-[3-(4-carbamoyl-1,3-oxazol-2-yl)-5-chloro-7-[(furan-2-ylmethyl) amino]furo[3,2-b]pyridin-2-yl]propan-2-yl]carbamate (50 mg, 31.46%) as a yellow solid.

### Step 3. Synthesis of 2-{2-[(2S)-2-aminopropyl]-5-chloro-7-[(furan-2-ylmethyl)amino]furo[3,2-b]pyridin-3-yl}-1,3-oxazole-5-carboxamide

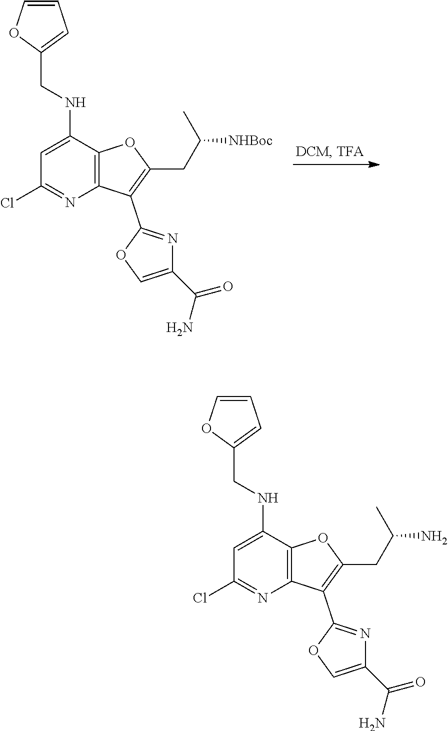 [SMILES: C[C@@H](Cc1oc2c(NCc3ccco3)cc(Cl)nc2c1-c1nc(C(N)=O)co1)NC(=O)OC(C)(C)C ; C[C@H](N)Cc1oc2c(NCc3ccco3)cc(Cl)nc2c1-c1nc(C(N)=O)co1]

A solution of tert-butyl N-[(2S)-1-[3-(5-carbamoyl-1,3-oxazol-2-yl)-5-chloro-7-[(furan-2-ylmethyl) amino]furo[3,2-b]pyridin-2-yl]propan-2-yl]carbamate (50 mg, 0.097 mmol, 1 equiv) and TEA (1 mL) in DCM (2 mL) was stirred for 1 h at room temperature under nitrogen atmosphere. The reaction mixture was concentrated under reduced pressure. The residue was purified by reversed-phase flash chromatography with the following conditions: column, C₁₈; mobile phase, MeCN in Water (0.1% FA), 10% to 100% gradient in 20 min; detector, UV 254 nm. This resulted in 2-{2-[(2S)-2-aminopropyl]-5-chloro-7-[(furan-2-ylmethyl)amino]furo[3,2-b]pyridin-3-yl}-1,3-oxazole-5-carboxamide (10 mg, 24.82%).

LC-MS (ES, m/z): [M+H]⁺=416.00

¹H NMR (400 MHz, Methanol-d₄) δ 8.54 (s, 1H), 7.98 (s, 1H), 7.77 (s, 1H), 7.46 (s, 1H), 6.71 (s, 1H), 6.41-6.32 (m, 2H), 4.57 (d, J=6.6 Hz, 3H), 3.90 (q, J=6.6 Hz, 1H), 3.57 (d, J=6.6 Hz, 2H), 1.41 (d, J=6.6 Hz, 3H), 1.36-1.20 (m, 1H), 0.87 (s, 1H).

### Example 30: Synthesis of 2-{2-[(2S)-2-aminopropyl]-5-chloro-7-[(furan-2-ylmethyl)amino]furo[3,2-b]pyridin-3-yl}-1,3-oxazole-5-carbonitrile (Compound 366)

### Step 1. Synthesis of tert-butyl N-[(2S)-1-[5-chloro-3-(5-cyano-1,3-oxazol-2-yl)-7-[(furan-2-ylmethyl)amino]furo[3,2-b]pyridin-2-yl]propan-2-yl]carbamate

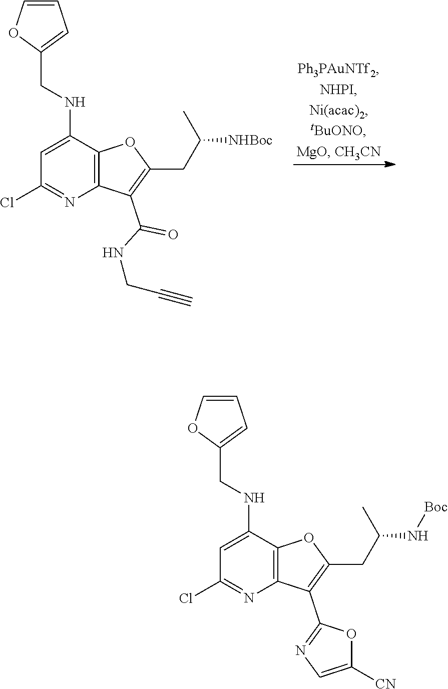 [SMILES: C#CCNC(=O)c1c(C[C@H](C)NC(=O)OC(C)(C)C)oc2c(NCc3ccco3)cc(Cl)nc12 ; C[C@@H](Cc1oc2c(NCc3ccco3)cc(Cl)nc2c1-c1ncc(C#N)o1)NC(=O)OC(C)(C)C]

A mixture of tert-butyl N-[(2S)-1-{5-chloro-7-[(furan-2-ylmethyl)amino]-3-[(prop-2-yn-1-yl) carbamoyl]furo[3,2-b]pyridin-2-yl}propan-2-yl]carbamate (242 mg, 0.497 mmol, 1 equiv), NHPI (0.02 g, 0.149 mmol, 0.3 equiv), tBuONO (0.15 g, 1.491 mmol, 3 equiv), MgO (0.06 g, 1.491 mmol, 3 equiv), nickel acetylacetonate (0.01 g, 0.037 mmol, 0.075 equiv), and Ph₃PAuNTf₂ (0.04 g, 0.050 mmol, 0.1 equiv) in CH₃CN (5 mL) was stirred for 3 h at 50° C. under nitrogen atmosphere. The residue was purified by reversed-phase flash chromatography with the following conditions: column, C₁₈; mobile phase, MeCN in Water (0.1% FA), 10% to 100% gradient in 20 min; detector, UV 254 nm. This resulted in tert-butyl N-[(2S)-1-[5-chloro-3-(5-cyano-1,3-oxazol-2-yl)-7-[(furan-2-ylmethyl)amino]furo[3,2-b]pyridin-2-yl]propan-2-yl]carbamate (60 mg, 24.25%) as a yellow solid.

### Step 2. Synthesis of 2-{2-[(2S)-2-aminopropyl]-5-chloro-7-[(furan-2-ylmethyl)amino]furo[3,2-b]pyridin-3-yl}-1,3-oxazole-5-carbonitrile

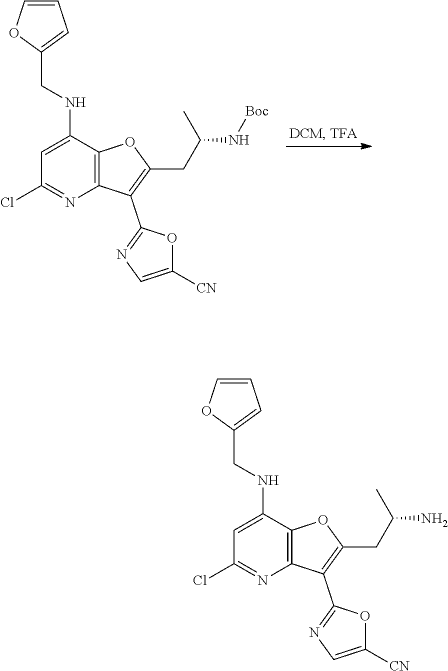 [SMILES: C[C@@H](Cc1oc2c(NCc3ccco3)cc(Cl)nc2c1-c1ncc(C#N)o1)NC(=O)OC(C)(C)C ; C[C@H](N)Cc1oc2c(NCc3ccco3)cc(Cl)nc2c1-c1ncc(C#N)o1]

A solution of tert-butyl N-[(2S)-1-[5-chloro-3-(5-cyano-1,3-oxazol-2-yl)-7-[(furan-2-ylmethyl)amino]furo[3,2-b]pyridin-2-yl]propan-2-yl]carbamate (60 mg, 0.120 mmol, 1 equiv) and TFA (1 mL) in DCM (2 mL) was stirred for 1 h at room temperature under nitrogen atmosphere. The residue was purified by reversed-phase flash chromatography with the following conditions: column, C₁₈; mobile phase, MeCN in Water (0.1% FA), 10% to 50% gradient in 10 min; detector, UV 254 nm. This resulted in 2-{2-[(2S)-2-aminopropyl]-5-chloro-7-[(furan-2-ylmethyl)amino]furo[3,2-b]pyridin-3-yl}-1,3-oxazole-5-carbonitrile (10 mg, 20.86%).

LC-MS (ES, m/z): [M+H]⁺=398.00

¹H NMR (400 MHz, Methanol-d₄) δ 8.10 (s, 1H), 7.45 (d, J=1.9 Hz, 1H), 6.70 (s, 1H), 6.40-6.32 (m, 2H), 4.57 (s, 2H), 3.49 (p, J=6.5 Hz, 1H), 3.43-3.32 (m, 2H), 1.20 (d, J=6.4 Hz, 3H).

### Example 31: Synthesis of 3-bromo-5-chloro-2-(cyclobutylmethoxy)-N-(thiophen-2-ylmethyl)furo[3,2-b]pyridin-7-amine (Compound 368)

### Step 1. Synthesis of tert-butyl N-[3-bromo-5-chloro-2-(cyclobutylmethoxy)furo[3,2-b]pyridin-7-yl]-N-(thiophen-2-ylmethyl)carbamate

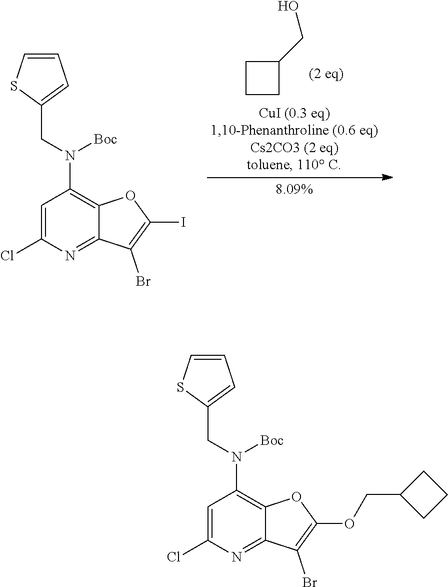 [SMILES: CC(C)(C)OC(=O)N(Cc1cccs1)c1cc(Cl)nc2c(Br)c(I)oc12 ; OCC1CCC1 ; CC(C)(C)OC(=O)N(Cc1cccs1)c1cc(Cl)nc2c(Br)c(OCC3CCC3)oc12]

A solution of tert-butyl N-{3-bromo-5-chloro-2-iodofuro[3,2-b]pyridin-7-yl}-N-(thiophen-2-ylmethyl) carbamate (800 mg, 1.404 mmol, 1 equiv), Cs₂CO₃ (915.16 mg, 2.808 mmol, 2 equiv) and 1,10-phenanthroline (151.85 mg, 0.842 mmol, 0.6 equiv) in toluene (20 mL) was treated with CuI (80.24 mg, 0.421 mmol, 0.3 equiv) for 1 min at room temperature under nitrogen atmosphere followed by the addition of cyclobutylmethanol (241.93 mg, 2.808 mmol, 2 equiv) dropwise at room temperature. The resulting mixture was stirred for 5 h at 110° C. under nitrogen atmosphere. The residue was purified by reversed-phase flash chromatography with the following conditions: column, C18; mobile phase, MeCN in water (0.1% FA), 10% to 100% gradient in 20 min; detector, UV 254 nm. This resulted in tert-butyl N-[3-bromo-5-chloro-2-(cyclobutylmethoxy)furo[3,2-b]pyridin-7-yl]-N-(thiophen-2-ylmethyl)carbamate (60 mg, 8.09%) as a yellow solid.

### Step 2. Synthesis of 3-bromo-5-chloro-2-(cyclobutylmethoxy)-N-(thiophen-2-ylmethyl)furo[3,2-b]pyridin-7-amine

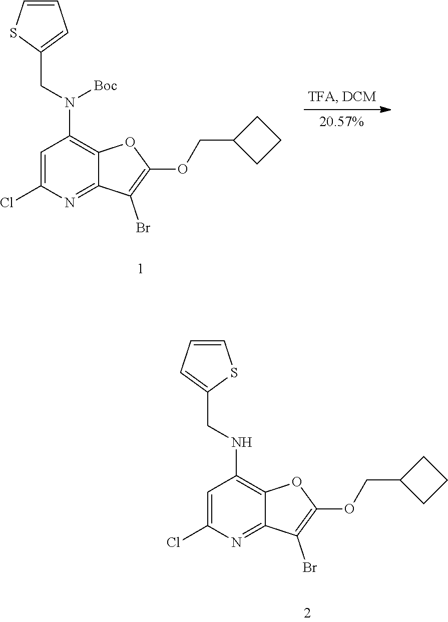 [SMILES: CC(C)(C)OC(=O)N(Cc1cccs1)c1cc(Cl)nc2c(Br)c(OCC3CCC3)oc12 ; Clc1cc(NCc2cccs2)c2oc(OCC3CCC3)c(Br)c2n1]

To a stirred solution of tert-butyl N-[3-bromo-5-chloro-2-(cyclobutylmethoxy)furo[3,2-b]pyridin-7-yl]-N-(thiophen-2-ylmethyl)carbamate (60 mg, 0.114 mmol, 1 equiv) in DCM (2 mL) were added TFA (1 mL) dropwise at room temperature under nitrogen atmosphere. The resulting mixture was stirred for 1 h at room temperature under nitrogen atmosphere, and then concentrated under reduced pressure. The residue was purified by reversed-phase flash chromatography with the following conditions: column, C18 silica gel; mobile phase, MeCN in Water (0.1% FA), 10% to 100% gradient in 20 min; detector, UV 254 nm. This resulted in 3-bromo-5-chloro-2-(cyclobutylmethoxy)-N-(thiophen-2-ylmethyl)furo[3,2-b]pyridin-7-amine (10 mg, 20.57%).

LC-MS (ES, m/z): [M+H]⁺=426.80

¹H NMR (400 MHz, Methanol-d₄) δ 7.30 (d, J=5.1 Hz, 1H), 7.05 (d, J=3.5 Hz, 1H), 6.97 (dd, J=5.1, 3.5 Hz, 1H), 6.53 (s, 1H), 4.74 (s, 2H), 4.43 (d, J=6.7 Hz, 2H), 2.79 (p, J=6.9, 6.2 Hz, 1H), 2.13 (q, J=9.6, 8.5 Hz, 2H), 2.05-1.87 (m, 4H).

### Example 32: Synthesis of 2-[(1S,2S)-2-amino-4,4-difluorocyclopentyl]-3-bromo-5-chloro-7-thenylamino-1-oxa-4-azaindene and 2-[(1R,2R)-2-amino-4,4-difluorocyclopentyl]-3-bromo-5-chloro-7-thenylamino-1-oxa-4-azaindene (Compounds 369 and 388)

Compounds 369 and 388 were obtained by racemic synthesis starting from 1,3-dioxoisoindol-2-yl 2-[(tert-butoxycarbonyl)amino]-4,4-difluorocyclopentane-1-carboxylate following General Scheme 13. The racemic product was separated into the individual enantiomers using the following chiral HPLC conditions: Column: CHIRALPAK IA, 3*25 cm, 5 μm; Mobile Phase A: Hex (0.1% 2M NH₃-MeOH)-HPLC, Mobile Phase B: EtOH-HPLC; Flow rate: 40 mL/min; Gradient: isocratic 50; Wave Length: 204/254 nm; RT1 (min): 4; RT2 (min): 10.5; Sample Solvent: MEOH; Injection Volume: 1 mL; Number Of Runs: 4.

This resulted in Compound 369 (70 mg, 38.73%) as the first eluting isomer and Compound 388 (70 mg, 38.73%) as the second eluting isomer. The absolute configuration was not determined.

### Example 33: Synthesis of Compounds 99, 100, 214, 215

Compounds 99, 100, 214, and 215, were made from AA-22 according to general scheme 8. The four diastereomers were separated by multiple rounds of chiral chromatographic separation at the penultimate step, and then individually deprotected. The absolute and relative configurations of the two stereogenic centers was not established. The chiral chromatography separation procedures are given below:

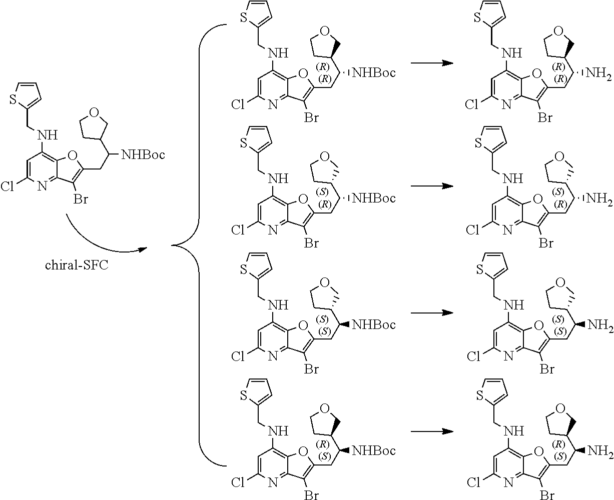

Step 1. Tert-butyl N-(2-{3-bromo-5-chloro-7-[(thiophen-2-ylmethyl)amino]furo[3,2-b]pyridin-2-yl}-1-(oxolan-3-yl)ethyl)carbamate (650 mg, 1.167 mmol, 1 equiv) was purified by Column: CHIRALPAK IG 3*25 cm, 5 μm Mobile Phase A: CO2, Mobile Phase B: ETOH:DCM=1:1; Flow rate: 100 mL/min; Gradient: isocratic 30% B; Column Temperature (° C.): 35; Back Pressure (bar): 100; Wave Length: 270/238 nm; RT1 (min): 7.12; RT2 (min): 8.57; Sample Solvent: MeOH:DCM=1:1-HPLC; Injection Volume: 0.35 mL; Number Of Runs: 49. This resulted in two isolated mixtures, Mixture A (300 mg, RT 7.12 min) and Mixture B (200 mg, 8.57 min). The mixtures were then further purified in steps 2 and 3.

Step 2. Mixture A (300 mg) was purified by Column: CHIRAL ART Cellulose-SC, 2*25 cm, 5 μm; Mobile Phase A: Hex (10 mM NH₃-MeOH), Mobile Phase B: IPA-HPLC; Flow rate: 20 mL/min; Gradient: 20% B to 20% B in 20 min; Wave Length: 209/236 nm; RT1 (min): 12.87; RT2 (min): 16.07; Sample Solvent: EtOH:DCM=1:1-HPLC; Injection Volume: 1 mL; Number Of Runs: 11 to afford 120 mg of as the first eluting (RT 12.87 min) compound and 120 mg of the second eluting compound (RT 16.07 min).

Step 3. Mixture B (200 mg) was purified by Column: CHIRAL ART Cellulose-SZ, 3*25 cm, 5 am; Mobile Phase A: Hex (0.1% 2M NH₃-MeOH)-HPLC, Mobile Phase B: IPA-HPLC; Flow rate: 40 mL/min; Gradient: 10% B to 10% B in 23.8 min; Wave Length: 212/226 nm; RT1 (min): 18.5; RT2 (min): 21.5; Sample Solvent: TPA:DCM=1:1-HPLC; Injection Volume: 0.9 mL; Number Of Runs: 20. This resulted in 70 mg of the first eluting isomer (RT 18.5 min) and 70 mg of the second eluting compound (RT 21.5 min).

Step 4. The first eluting compound of step 2 was subsequently deprotected with TFA to afford Compound 99. The second eluting compound of step 2 was subsequently deprotected with TFA to afford Compound 100. The first eluting compound of step 3 was subsequently deprotected with TFA to afford Compound 214. The second eluting compound of step 3 was subsequently deprotected with TFA to afford Compound 215.

The following compounds were made using the above synthesis schemes and have the physical criteria set forth in Table 3.

TABLE 3

| | General | | |
|---|---|---|---|
| | Synthesis | | LC/MS |
| Compound# | Scheme | ¹H NMR data | Ion |
| 1 | 2, 5 | (400 MHz, Methanol-d4) δ 8.52-8.46 (m, 2H), 7.47- | 317.0 |
| | | 7.41 (m, 2H), 6.67 (s, 1H), 6.42 (d, J = 1.5 Hz, 1H), 4.69 (s, | |
| | | 2H), 3.76-3.66 (m, 1H), 3.22-3.07 (m, 2H), 1.36 (d, J = | |
| | | 6.6 Hz, 3H). | |
| 2 | 2, 5 | 1(400 MHz, Methanol-d4) δ 7.41-7.30 (m, 4H), 7.30- | 316.0 |
| | | 7.21 (m, 1H), 6.57 (s, 1H), 6.40 (d, J = 1.2 Hz, 1H), 4.57 (s, | |
| | | 2H), 3.53 (h, J = 6.5 Hz, 1H), 3.01 (d, J = 6.6 Hz, 2H), 1.26 | |
| | | (d, J = 6.5 Hz, 3H). | |
| 3 | 2, 5 | (400 MHz, Methanol-d4) δ 8.52-8.45 (m, 2H), 7.46- | 331.0 |
| | | 7.40 (m, 2H), 6.35 (d, J = 1.1 Hz, 1H), 4.66 (s, 2H), 3.39- | |
| | | 3.30 (m, 1H), 2.87 (h, J = 7.9, 7.2 Hz, 2H), 2.16 (s, 3H), | |
| | | 1.14 (dd, J = 6.4, 1.2 Hz, 3H). | |
| 4 | 2, 5 | (400 MHz, Methanol-d4) δ 8.52-8.45 (m, 2H), 7.46- | 331.0 |
| | | 7.40 (m, 2H), 6.35 (d, J = 1.1 Hz, 1H), 4.66 (s, 2H), 3.39- | |
| | | 3.30 (m, 1H), 2.87 (h, J = 7.9, 7.2 Hz, 2H), 2.16 (s, 3H), | |
| | | 1.14 (dd, J = 6.4, 1.2 Hz, 3H). | |
| 5 | 6 | (400 MHz, DMSO-d6) δ 8.54-8.48 (m, 2H), 7.97 (q, J = | 274.0 |
| | | 1.2 Hz, 1H), 7.85 (t, J = 6.4 Hz, 1H), 7.37-7.31 (m, 2H), | |
| | | 6.39 (s, 1H), 4.58 (d, J = 6.4 Hz, 2H), 2.12 (d, J = 1.3 Hz, 3H). | |
| 6 | 6 | (300 MHz, DMSO-d6) δ 8.52 (d, J = 5.3 Hz, 2H), 8.20 (d, | 260.0 |
| | | J = 2.2 Hz, 1H), 7.95 (q, J = 6.4, 5.3 Hz, 1H), 7.39-7.29 (m, | |
| | | 2H), 6.93 (d, J = 2.2 Hz, 1H), 6.40 (s, 1H), 4.59 (d, J = 6.4 | |
| | | Hz, 2H). | |
| 7 | 6 | (400 MHz, DMSO-d6) δ 8.53-8.47 (m, 2H), 7.75 (t, J = | 331.2 |
| | | 6.5 Hz, 1H), 7.37-7.31 (m, 2H), 6.33 (d, J = 5.8 Hz, 1H), | |
| | | 4.57 (d, J = 6.4 Hz, 2H), 3.26 (d, J = 6.4 Hz, 1H), 2.89- | |
| | | 2.71 (m, 2H), 2.06 (d, J = 4.9 Hz, 3H), 1.04 (d, J = 6.3 Hz, 3H). | |
| 8 | 6 | (400 MHz, Methanol-d4) δ 7.40-7.29 (m, 4H), 7.29- | 330.0 |
| | | 7.20 (m, 1H), 6.38 (s, 1H), 4.56 (s, 2H), 3.39 (d, J = 6.5 Hz, | |
| | | 0H), 2.95-2.81 (m, 2H), 2.15 (s, 3H), 1.17 (d, J = 6.4 Hz, 3H). | |
| 9 | 6 | (400 MHz, Methanol-d4) δ 7.76 (dd, J = 3.4, 1.2 Hz, 1H), | 337.0 |
| | | 7.52 (dd, J = 3.3, 1.2 Hz, 1H), 6.49 (d, J = 1.2 Hz, 1H), 3.41 | |
| | | (q, J = 6.5 Hz, 1H), 3.31 (s, 0H), 2.90 (s, 2H), 2.90 (q, J = | |
| | | 14.6 Hz, 1H), 2.16 (s, 3H), 1.17 (dd, J = 6.5, 1.5 Hz, 3H) | |
| 10 | 6 | (400 MHz, Methanol-d4) δ 7.44 (d, J = 2.3 Hz, 1H), 6.55 | 320.0 |
| | | (d, J = 2.2 Hz, 1H), 6.46-6.18 (m, 2H), 4.54 (d, J = 2.2 Hz, | |
| | | 2H), 3.39 (s, 2H), 2.87 (dd, J = 6.7, 3.3 Hz, 2H), 2.15 (d, J = | |
| | | 2.2 Hz, 3H), 1.16 (dd, J = 6.5, 2.2 Hz, 3H). | |
| 11 | 6 | (400 MHz, Methanol-d4) δ 7.29 (dd, J = 5.0, 1.3 Hz, 1H), | 336.0 |
| | | 7.10-7.01 (m, 1H), 6.96 (dd, J = 5.1, 3.5 Hz, 1H), 6.51 (s, | |
| | | 1H), 4.75 (s, 2H), 3.42 (q, J = 6.5 Hz, 1H), 2.92-2.80 (m, | |
| | | 2H), 2.16 (s, 3H), 1.18 (d, J = 6.5 Hz, 3H). | |
| 12 | 6 | (400 MHz, Methanol-d4) δ 8.53 (s, 1H), 6.73 (t, J = 3.7 | 354.0 |
| | | Hz, 1H), 6.56 (d, J = 1.4 Hz, 1H), 6.40 (dt, J = 3.7, 1.7 Hz, | |
| | | 1H), 4.70-4.53 (m, 2H), 3.68 (q, J = 6.7 Hz, 1H), 3.08 | |
| | | (qd, J = 15.0, 6.6 Hz, 2H), 2.18 (d, J = 1.5 Hz, 3H), 1.33 | |
| | | (dd, J = 6.6, 1.5 Hz, 3H). | |
| 13 | 6 | (400 MHz, Methanol-d4) δ 7.29 (dd, J = 5.1, 1.2 Hz, 1H), | 350.0 |
| | | 7.06 (dq, J = 3.3, 1.1 Hz, 1H), 6.96 (dd, J = 5.1, 3.5 Hz, | |
| | | 1H), 6.50 (s, 1H), 4.75 (d, J = 1.0 Hz, 2H), 3.18-3.11 (m, | |
| | | 1H), 2.93 (dd, J = 14.9, 5.6 Hz, 1H), 2.82 (dd, J = 14.9, 7.5 | |
| | | Hz, 1H), 2.16 (s, 3H), 1.60-1.39 (m, 1H), 0.99 (t, J = 7.5 | |
| | | Hz, 3H). | |
| 14 | 6 | (400 MHz, Methanol-d4) δ 7.29 (dd, J = 5.1, 1.2 Hz, 1H), | 376.0 |
| | | 7.06 (dq, J = 3.3, 1.1 Hz, 1H), 6.97 (dd, J = 5.1, 3.5 Hz, | |
| | | 1H), 6.51 (s, 1H), 3.37 (q, J = 6.7 Hz, 1H), 3.02 (dd, J = | |
| | | 14.9, 5.5 Hz, 1H), 2.86 (dd, J = 14.9, 7.8 Hz, 1H), 2.16 (s, | |
| | | 3H), 1.40 (h, J = 7.2 Hz, 2H), 0.79 (d, J = 11.7 Hz, 1H), | |
| | | 0.55-0.46 (m, 1H), 0.50 (s, 1H), 0.18-0.04 (m, 1H), | |
| | | 0.08 (s, 1H). | |
| 15 | 6, 7 | (400 MHz, DMSO-d6) δ 7.84 (s, 1H), 7.39 (d, J = 5.1 Hz, | 327.0 |
| | | 1H), 7.12-7.07 (m, 2H), 6.97 (dt, J = 4.9, 2.5 Hz, 1H), | |
| | | 4.75 (d, J = 6.1 Hz, 2H), 3.22 (s, 1H), 2.76 (t, J = 7.8 Hz, | |
| | | 1H), 2.11 (d, J = 2.1 Hz, 2H), 1.10-0.99 (m, 3H). | |
| 16 | 3 | 1H NMR (400 MHz, DMSO-d6) δ 8.38 (d, J = 4.9 Hz, 1H), | 356.1 |
| | | 8.07 (t, J = 6.2 Hz, 1H), 7.41 (dd, J = 5.1, 1.3 Hz, 1H), 7.11 | |
| | | (d, J = 3.5 Hz, 1H), 6.99 (dd, J = 5.1, 3.4 Hz, 1H), 6.64 (s, | |
| | | 1H), 4.74 (d, J = 6.1 Hz, 2H), 3.52 (d, J = 6.6 Hz, 1H), 3.13- | |
| | | 2.95 (m, 2H), 1.17 (d, J = 6.4 Hz, 3H). | |
| 17 | 6 | (400 MHz, DMSO-d6) δ 7.94 (t, J = 6.2 Hz, 1H), 7.40 (dd, | 400.0 |
| | | J = 5.1, 1.3 Hz, 1H), 7.10 (dd, J = 3.5, 1.2 Hz, 1H), 6.98 (dd, | |
| | | J = 5.1, 3.5 Hz, 1H), 6.61 (s, 1H), 4.72 (d, J = 6.2 Hz, 2H), | |
| | | 3.32 (s, 1H), 3.33-3.25 (m, 1H), 2.83 (qd, J = 14.6, 6.7 | |
| | | Hz, 2H), 1.04 (d, J = 6.3 Hz, 3H). | |
| 18 | 8, 16 | (400 MHz, Methanol-d4) δ 7.31 (dd, J = 5.1, 1.2 Hz, 1H), | 346.9 |
| | | 7.10 (dd, J = 3.5, 1.2 Hz, 1H), 6.98 (dd, J = 5.1, 3.5 Hz, | |
| | | 1H), 6.70 (d, J = 1.7 Hz, 1H), 4.78 (s, 2H), 4.25 (dp, J = | |
| | | 10.2, 6.5 Hz, 1H), 3.40 (dd, J = 18.0, 6.3 Hz, 1H), 3.35 (s, | |
| | | 1H), 3.10 (dd, J = 17.9, 10.3 Hz, 1H), 1.50 (d, J = 6.6 Hz, 3H). | |
| 19 | 2, 3 | (300 MHz, DMSO-d6):8 7.66 (d, J = 6.6 Hz, 3H), 7.38 (dd, | 361.4 |
| | | J = 5.1, 1.3 Hz, 3H), 7.07 (d, J = 3.4 Hz, 3H), 6.97 (dd, J = | |
| | | 5.1, 3.4 Hz, 3H), 6.77 (s, 1H), 6.44 (s, 3H), 6.05 (s, 1H), | |
| | | 4.67 (d, J = 6.2 Hz, 6H), 3.91 (d, J = 8.0 Hz, 2H), 3.24 (d, | |
| | | J = 6.3 Hz, 1H), 3.00 (dd, J = 14.6, 6.0 Hz, 1H), 2.81 (dt, J = | |
| | | 15.0, 7.0 Hz, 3H), 1.79 (s, 4H), 1.23 (s, 1H), 1.17-0.99 | |
| | | (m, 7H), 0.86-0.73 (m, 6H). | |
| 20 | 6 | (300 MHz, DMSO-d6) δ 8.02-7.92 (m, 1H), 7.41 (dd, J = | 417.9 |
| | | 5.1, 1.3 Hz, 1H), 7.11 (dd, J = 3.5, 1.2 Hz, 1H), 6.98 (dd, | |
| | | J = 5.1, 3.4 Hz, 1H), 6.62 (s, 1H), 4.93 (s, 1H), 4.73 (d, J = | |
| | | 6.1 Hz, 2H), 4.28 (s, 1H), 3.47-3.30 (m, 2H), 3.25 (s, 1H), | |
| | | 3.01 (dd, J = 15.0, 5.5 Hz, 1H), 2.83 (dd, J = 14.9, 7.8 Hz, 1H). | |
| 21 | 6 | (400 MHz, Methanol-d4) δ 8.53 (s, 1H), 7.31 (dd, J = 5.1, | 371.9 |
| | | 1.2 Hz, 1H), 7.09 (d, J = 3.5 Hz, 1H), 6.98 (dd, J = 5.1, 3.4 | |
| | | Hz, 1H), 6.62 (dd, J = 3.6, 1.6 Hz, 1H), 4.76 (s, 2H), 3.75 | |
| | | (q, J = 6.3 Hz, 1H), 3.60 (q, J = 5.9, 5.5 Hz, 2H), 3.23 (dd, | |
| | | J = 15.4, 5.9 Hz, 1H), 3.15 (dd, J = 15.4, 6.5 Hz, 1H). | |
| 22 | 6 | (300 MHz, DMSO-d6) δ 7.83 (t, J = 6.1 Hz, 1H), 7.60 (dd, | 400.0 |
| | | J = 1.8, 0.9 Hz, 1H), 6.66 (s, 1H), 6.44-6.34 (m, 2H), 4.53 | |
| | | (d, J = 6.0 Hz, 2H), 3.47-3.30 (m, 2H), 3.25 (t, J = 6.4 Hz, | |
| | | 1H), 3.01 (dd, J = 15.0, 5.5 Hz, 1H), 2.82 (dd, J = 15.0, 7.8 | |
| | | Hz, 1H). | |
| 23 | 6 | (400 MHz, Methanol-d4) δ 7.45 (d, J = 1.7 Hz, 1H), 6.70 | 355.9 |
| | | (s, 1H), 6.36 (dd, J = 8.2, 2.7 Hz, 2H), 4.56 (s, 2H), 3.81 | |
| | | (dd, J = 11.3, 3.4 Hz, 1H), 3.67 (ddd, J = 26.2, 10.7, 6.2 | |
| | | Hz, 2H), 3.32-3.18 (m, 2H). | |
| 24 | 6 | (400 MHz, DMSO-d6) δ 7.84 (t, J = 6.1 Hz, 1H), 7.60 (d, | 470.0 |
| | | J = 1.8 Hz, 1H), 6.66 (s, 1H), 6.40 (t, J = 2.6 Hz, 1H), 6.37 (d, | |
| | | J = 3.3 Hz, 1H), 4.53 (d, J = 6.0 Hz, 2H), 3.05-2.90 (m, | |
| | | 2H), 2.40 (dddd, J = 23.2, 11.5, 8.6, 3.9 Hz, 2H). | |
| 25 | 6 | (400 MHz, DMSO-d6) δ 8.18-8.02 (m, 2H), 7.96 (t, J = | 424.1 |
| | | 6.2 Hz, 1H), 7.43 (dd, J = 5.1, 1.3 Hz, 1H), 7.12 (dd, J = | |
| | | 3.4, 1.2 Hz, 1H), 7.00 (dd, J = 5.1, 3.5 Hz, 1H), 6.69 (s, | |
| | | 1H), 4.77 (d, J = 6.1 Hz, 2H), 3.89 (t, J = 6.3 Hz, 1H), 3.27 | |
| | | (s, 2H), 2.83 (dt, J = 11.1, 5.6 Hz, 1H), 2.73 (ddd, J = 15.8, | |
| | | 11.0, 5.8 Hz, 1H). | |
| 26 | 6 | (400 MHz, DMSO-d6) δ 7.84 (t, J = 6.1 Hz, 1H), 7.60 (d, | 454.1 |
| | | J = 1.8 Hz, 1H), 6.66 (s, 1H), 6.39 (dd, J = 10.0, 2.8 Hz, 2H), | |
| | | 4.53 (d, J = 6.0 Hz, 2H), 3.52-3.48 (m, 1H), 3.00-2.87 | |
| | | (m, 2H), 2.40 (dqd, J = 15.1, 7.9, 4.3 Hz, 2H). | |
| 27 | 6 | (400 MHz, Methanol-d4) δ 7.45 (d, J = 1.8 Hz, 1H), 6.67 | 408.1 |
| | | (s, 1H), 6.39-6.30 (m, 2H), 4.55 (s, 2H), 3.89-3.78 (m, | |
| | | 1H), 3.20 (qd, J = 15.4, 6.4 Hz, 2H), 2.62 (tt, J = 14.8, 4.3 | |
| | | Hz, 1H), 2.57-2.43 (m, 1H). | |
| 28 | 6 | (400 MHz, DMSO-d6) δ 8.31 (d, J = 10.6 Hz, 1H), 8.03 (d, | 396.1 |
| | | J = 6.7 Hz, 1H), 7.41 (dd, J = 5.1, 1.3 Hz, 1H), 7.10 (d, J = | |
| | | 3.5 Hz, 1H), 6.99 (dd, J = 5.1, 3.4 Hz, 1H), 6.62 (s, 1H), | |
| | | 4.73 (d, J = 6.2 Hz, 2H), 3.36 (s, 1H), 3.05 (dd, J = 15.0, | |
| | | 5.4 Hz, 1H), 2.96-2.88 (m, 1H), 1.34 (dq, J = 34.3, 7.2 | |
| | | Hz, 2H), 0.78 (d, J = 5.3 Hz, 1H), 0.42 (qd, J = 6.3, 3.3 Hz, | |
| | | 2H), 0.08 (d, J = 10.5 Hz, 1H). | |
| 29 | 6 | (400 MHz, DMSO-d6) δ 7.90 (t, J = 6.1 Hz, 1H), 7.60 (dd, | 380.2 |
| | | J = 1.9, 0.9 Hz, 1H), 6.66 (s, 1H), 6.48-6.31 (m, 2H), 4.54 | |
| | | (d, J = 6.0 Hz, 2H), 3.34 (h, J = 5.9 Hz, 1H), 3.03 (dd, J = | |
| | | 15.0, 5.5 Hz, 1H), 2.92 (dd, J = 15.1, 7.6 Hz, 1H), 1.34 (dq, | |
| | | J = 33.6, 7.1 Hz, 2H), 0.79 (qd, J = 8.3, 7.9, 2.4 Hz, 1H), | |
| | | 0.50-0.33 (m, 2H), 0.14-0.04 (m, 1H). | |
| 30 | 6 | (400 MHz, Methanol-d4) δ 7.29 (dd, J = 5.1, 1.2 Hz, 1H), | 404.0 |
| | | 7.12-7.05 (m, 1H), 6.96 (dd, J = 5.1, 3.5 Hz, 1H), 6.51 (s, | |
| | | 1H), 4.74 (s, 2H), 3.57 (ddd, J = 12.8, 7.8, 5.1 Hz, 1H), | |
| | | 3.06-2.86 (m, 2H), 2.50-2.24 (m, 2H), 2.16 (s, 3H). | |
| 31 | 14 | (400 MHz, Methanol-d4) δ 7.31 (dd, J = 5.1, 1.2 Hz, 1H), | 368.0 |
| | | 7.08 (dq, J = 3.2, 1.0 Hz, 1H), 6.98 (dd, J = 5.1, 3.5 Hz, | |
| | | 1H), 6.61 (s, 1H), 4.80-4.77 (m, 2H), 4.75-4.67 (m, | |
| | | 1H), 4.66-4.53 (m, 1H), 3.84 (p, J = 6.5 Hz, 1H), 3.21 (p, | |
| | | J = 8.9 Hz, 2H), 2.20 (s, 3H), 2.17-2.00 (m, 2H). | |
| 32 | 2 | (300 MHz, DMSO-d6): δ 7.93 (s, 1H), 7.80 (t, J = 6.2 Hz, | 305.0 |
| | | 1H), 7.39 (dd, J = 5.1, 1.3 Hz, 1H), 7.12-7.05 (m, 1H), | |
| | | 6.98 (dd, J = 5.0, 3.5 Hz, 1H), 6.53 (s, 1H), 4.70 (d, J = 6.3 | |
| | | Hz, 2H), 2.08 (s, 0H), 1.85 (ddd, J = 13.5, 8.2, 5.4 Hz, 1H), | |
| | | 0.95-0.78 (m, 4H). | |
| 33 | 2 | (300 MHz, DMSO-d6): δ 7.92 (s, 1H), 7.69-7.56 (m, 2H), | 289.1 |
| | | 6.57 (s, 1H), 6.43-6.30 (m, 2H), 4.50 (d, J = 6.1 Hz, 2H), | |
| | | 1.85 (tt, J = 8.2, 5.3 Hz, 1H), 0.95-0.78 (m, 4H). | |
| 34 | 6 | (400 MHz, DMSO-d6) δ 7.79 (t, J = 6.1 Hz, 1H), 7.59 (dd, | 384.1 |
| | | J = 1.9, 0.9 Hz, 1H), 6.64 (s, 1H), 6.38 (ddd, J = 13.8, 3.2, | |
| | | 1.3 Hz, 2H), 4.52 (d, J = 6.0 Hz, 2H), 3.32-3.23 (m, 1H), | |
| | | 2.90-2.73 (m, 2H), 1.03 (d, J = 6.3 Hz, 3H). | |
| 35 | 7 | (300 MHz, Methanol-d4) δ 7.34 (dd, J = 5.1, 1.1 Hz, 1H), | 392.9 |
| | | 7.13 (td, J = 3.3, 1.4 Hz, 2H), 7.01 (dd, J = 5.1, 3.5 Hz, 1H), | |
| | | 4.85 (s, 2H), 3.84 (p, J = 6.7 Hz, 1H), 3.39-3.17 (m, 2H), | |
| | | 1.40 (d, J = 6.6 Hz, 3H). | |
| 36 | 7 | (300 MHz, Methanol-d4) δ 8.55 (s, 1H), 7.48 (t, J = 1.4 | 374.9 |
| | | Hz, 1H), 7.19 (s, 1H), 6.39 (d, J = 1.4 Hz, 2H), 4.64 (s, 2H), | |
| | | 3.80 (q, J = 6.7 Hz, 1H), 3.34-3.15 (m, 2H), 1.39 (d, J = | |
| | | 6.6 Hz, 3H). | |
| 37 | 6 | (400 MHz, DMSO-d6) δ 7.96 (t, J = 6.2 Hz, 1H), 7.41 (dd, | 430.0 |
| | | J = 5.0, 1.3 Hz, 1H), 7.10 (dd, J = 3.4, 1.2 Hz, 1H), 6.98 (dd, | |
| | | J = 5.1, 3.4 Hz, 1H), 6.61 (s, 1H), 4.72 (d, J = 6.1 Hz, 2H), | |
| | | 3.36-3.25 (m, 1H), 3.26 (s, 3H), 3.23 (d, J = 1.2 Hz, 1H), | |
| | | 2.96 (dd, J = 14.8, 5.4 Hz, 1H), 2.78 (dd, J = 14.8, 7.7 Hz, | |
| | | 1H), 2.01 (s, 2H). | |
| 38 | 6 | (400 MHz, DMSO-d6) δ 7.81 (t, J = 6.1 Hz, 1H), 7.60 (dd, | 414.1 |
| | | J = 1.8, 0.9 Hz, 1H), 6.65 (s, 1H), 6.43-6.34 (m, 2H), 4.52 | |
| | | (d, J = 6.1 Hz, 2H), 3.35-3.25 (m, 1H), 3.26 (s, 3H), 2.95 | |
| | | (dd, J = 14.7, 5.3 Hz, 1H), 2.77 (dd, J = 14.8, 7.7 Hz, 1H), | |
| | | 1.82 (s, 3H). | |
| 39 | 6 | (300 MHz, Methanol-d4) δ 7.47 (dd, J = 1.9, 0.9 Hz, 1H), | 404.0 |
| | | 6.67 (s, 1H), 6.37 (ddd, J = 9.9, 3.2, 1.3 Hz, 2H), 4.57 (s, | |
| | | 2H), 4.54-4.24 (m, 2H), 3.52 (d, J = 19.1 Hz, 1H), 3.26- | |
| | | 2.90 (m, 2H). | |
| 40 | 7 | 1H NMR (400 MHz, Methanol-d4) δ 7.28 (s, 1H), 7.05 (d, | 326 |
| | | J = 1.3 Hz, 2H), 6.99-6.95 (m, 3H), 6.72 (s, 4H), 4.76 (d, | |
| | | J = 1.0 Hz, 8H), 3.48 (s, 2H), 3.38 (p, J = 6.6 Hz, 4H), 2.94- | |
| | | 2.81 (m, 8H), 2.18 (s, 11H), 1.15 (d, J = 6.4 Hz, 11H). | |
| 41 | 6 | (400 MHz, Methanol-d4) δ 7.29 (dd, J = 5.1, 1.2 Hz, 1H), | 368.0 |
| | | 7.06 (dt, J = 3.5, 1.2 Hz, 1H), 6.97 (dd, J = 5.1, 3.5 Hz, 1H), | |
| | | 6.48 (d, J = 15.6 Hz, 1H), 4.83-4.65 (m, 2H), 4.55 (ddt, | |
| | | J = 47.5, 11.6, 6.2 Hz, 1H), 3.29-3.18 (m, 1H), 3.04 (dd, | |
| | | J = 15.0, 4.5 Hz, 1H), 2.83 (dd, J = 15.0, 8.6 Hz, 1H), 2.16 | |
| | | (d, J = 3.8 Hz, 3H), 1.50-1.22 (m, 3H). | |
| 42 | 6 | 1H NMR (400 MHz, DMSO-d6) δ 8.23-8.11 (m, 1H), | 368 |
| | | 7.78 (d, J = 3.3 Hz, 1H), 7.65 (d, J = 3.3 Hz, 1H), 6.62 (s, | |
| | | 1H), 4.87 (d, J = 6.3 Hz, 2H), 4.54 (dq, J = 47.9, 6.2 Hz, | |
| | | 1H), 3.06 (dd, J = 15.0, 4.4 Hz, 1H), 2.81 (dd, J = 15.0, 8.9 | |
| | | Hz, 1H), 1.33 (dd, J = 24.8, 6.2 Hz, 3H). | |
| 43 | 6 | (400 MHz, DMSO-d6) δ 7.98 (t, J = 6.1 Hz, 1H), 7.41 (dd, | 432.1 |
| | | J = 5.1, 1.3 Hz, 1H), 7.11 (dd, J = 3.4, 1.2 Hz, 1H), 6.98 (dd, | |
| | | J = 5.1, 3.4 Hz, 1H), 6.62 (s, 1H), 4.72 (d, J = 6.1 Hz, 2H), | |
| | | 4.53 (dtd, J = 48.3, 6.2, 2.1 Hz, 1H), 3.21 (d, J = 11.3 Hz, | |
| | | 1H), 3.06 (dd, J = 14.9, 4.4 Hz, 1H), 2.87-2.68 (m, 1H), | |
| | | 1.33 (dd, J = 24.8, 6.2 Hz, 3H). | |
| 44 | 6 | (400 MHz, Methanol-d4) δ 7.77 (d, J = 3.3 Hz, 1H), 7.56 | 435.0 |
| | | (d, J = 3.3 Hz, 1H), 6.65 (s, 1H), 4.95 (s, 2H), 4.82 (d, J = | |
| | | 6.0 Hz, 1H), 4.70 (q, J = 6.1 Hz, 1H), 3.69 (dq, J = 12.6, 6.1 | |
| | | Hz, 1H), 3.34 (d, J = 6.0 Hz, 1H), 3.22 (dd, J = 15.7, 7.2 Hz, | |
| | | 1H), 1.48 (dd, J = 24.7, 6.4 Hz, 3H). | |
| 45 | 6 | (300 MHz, Methanol-d4) δ 8.53 (d, J = 1.9 Hz, 1H), 7.79 | 402.8 |
| | | (d, J = 3.3 Hz, 1H), 7.58 (d, J = 3.3 Hz, 1H), 6.66 (d, J = 1.5 | |
| | | Hz, 1H), 4.97 (s, 2H), 3.77 (q, J = 6.6 Hz, 1H), 3.23 (t, J = | |
| | | 7.0 Hz, 2H), 1.37 (d, J = 6.6 Hz, 3H). | |
| 46 | | (300 MHz, Methanol-d4) δ 8.53 (s, 1H), 7.43-7.21 (m, | 423.9 |
| | | 5H), 6.49 (d, J = 1.0 Hz, 1H), 4.57 (s, 2H), 3.71 (s, 1H), | |
| | | 3.55 (dd, J = 10.2, 3.5 Hz, 1H), 3.47-3.35 (m, 1H), 3.40 | |
| | | (s, 3H), 3.19 (tt, J = 15.1, 7.5 Hz, 2H). | |
| 47 | 6 | (300 MHz, Methanol-d4) δ 8.53 (s, 1H), 7.43-7.21 (m, | 423.9 |
| | | 5H), 6.49 (d, J = 1.0 Hz, 1H), 4.57 (s, 2H), 3.71 (s, 1H), | |
| | | 3.55 (dd, J = 10.2, 3.5 Hz, 1H), 3.47-3.35 (m, 1H), 3.40 | |
| | | (s, 3H), 3.19 (tt, J = 15.1, 7.5 Hz, 2H). | |
| 48 | 6 | (300 MHz, Methanol-d4) δ 8.57-8.45 (m, 2H), 7.48- | 424.9 |
| | | 7.40 (m, 2H), 6.44 (s, 1H), 4.67 (s, 2H), 3.48-3.35 (m, | |
| | | 2H), 3.36 (s, 3H), 3.08 (dd, J = 14.9, 5.8 Hz, 1H), 2.95 (dd, | |
| | | J = 14.9, 7.0 Hz, 1H). | |
| 49 | 6 | (300 MHz, Methanol-d4) δ 8.55 (s, 0H), 7.79 (d, J = 3.3 | 432.8 |
| | | Hz, 1H), 7.57 (d, J = 3.3 Hz, 1H), 6.65 (s, 1H), 3.79-3.70 | |
| | | (m, 1H), 3.58 (dd, J = 10.2, 3.8 Hz, 1H), 3.49-3.38 (m, | |
| | | 1H), 3.43 (s, 3H), 3.22 (tt, J = 15.2, 7.6 Hz, 2H). | |
| 50 | 6 | (400 MHz, DMSO-d6) δ 8.26 (s, 1H), 8.00 (t, J = 6.2 Hz, | 386.1 |
| | | 1H), 7.41 (dd, J = 5.1, 1.2 Hz, 1H), 7.11 (d, J = 3.4 Hz, 1H), | |
| | | 6.99 (dd, J = 5.1, 3.4 Hz, 1H), 6.62 (s, 1H), 4.73 (d, J = 6.2 | |
| | | Hz, 2H), 3.40 (d, J = 6.4 Hz, 1H), 3.39-3.31 (m, 2H), 3.27 | |
| | | (s, 3H), 3.02 (dd, J = 15.0, 5.9 Hz, 1H), 2.88 (dd, J = 15.0, | |
| | | 7.5 Hz, 1H). | |
| 51 | 6 | (300 MHz, DMSO-d6) δ 7.98 (t, J = 6.2 Hz, 1H), 7.43- | 380.2 |
| | | 7.21 (m, 5H), 6.50 (s, 1H), 4.56 (d, J = 6.2 Hz, 2H), 3.50- | |
| | | 3.28 (m, 2H), 3.05 (dd, J = 15.0, 6.0 Hz, 1H), 2.92 (dd, J = | |
| | | 15.0, 7.3 Hz, 1H). | |
| 52 | 6 | (400 MHz, DMSO-d6) δ 8.23 (s, 1H), 7.84 (t, J = 6.1 Hz, | 370.1 |
| | | 1H), 7.60 (dd, J = 1.9, 0.9 Hz, 1H), 6.66 (s, 1H), 6.44- | |
| | | 6.33 (m, 2H), 4.53 (d, J = 6.0 Hz, 2H), 3.37 (s, 1H), 3.36- | |
| | | 3.31 (m, 2H), 3.27 (s, 3H), 2.99 (dd, J = 14.9, 5.6 Hz, 1H), | |
| | | 2.83 (dd, J = 15.0, 7.6 Hz, 1H). | |
| 53 | 6 | (300 MHz, DMSO-d6) δ 8.18 (d, J = 6.6 Hz, 3H), 7.79 (d, | 387.1 |
| | | J = 3.2 Hz, 2H), 7.67 (d, J = 3.3 Hz, 2H), 6.67 (s, 2H), 4.90 | |
| | | (d, J = 6.3 Hz, 4H), 3.59-3.49 (m, 3H), 3.48-3.31 (m, | |
| | | 3H), 3.06 (qd, J = 15.2, 6.8 Hz, 3H). | |
| 54 | 6 | (400 MHz, DMSO-d6) δ 8.38 (d, J = 4.9 Hz, 1H), 7.91 (t, | 340.1 |
| | | J = 6.2 Hz, 1H), 7.61 (dd, J = 5.1, 1.3 Hz, 1H), 6.71 (m, 1H), | |
| | | 6.44 (s, 2H), 4.54 (d, J = 6.1 Hz, 2H), 3.51 (d, J = 6.6 Hz, | |
| | | 1H), 3.12-2.96 (m, 2H), 1.17 (d, J = 6.4 Hz, 3H). | |
| 55 | 6 | (300 MHz, DMSO-d6) δ 8.28 (s, 1H), 7.99 (t, J = 6.0 Hz, | 441.8 |
| | | 2H), 7.42 (dd, J = 5.1, 1.3 Hz, 2H), 7.11 (d, J = 3.5 Hz, 2H), | |
| | | 6.99 (dd, J = 5.1, 3.4 Hz, 2H), 6.62 (s, 2H), 4.74 (d, J = 6.2 | |
| | | Hz, 4H), 3.03 (dd, J = 14.9, 5.6 Hz, 2H), 2.90 (dd, J = 14.7, | |
| | | 7.7 Hz, 2H), 1.32 (ddd, J = 27.5, 14.1, 7.2 Hz, 3H), 0.79 (s, | |
| | | 2H), 0.42 (d, J = 8.0 Hz, 4H), 0.07 (s, 3H). | |
| 56 | 6 | (300 MHz, DMSO-d6): δ 7.81 (t, J = 6.1 Hz, 1H), 7.61 (dd, | 425.9 |
| | | J = 1.9, 0.9 Hz, 1H), 6.67 (s, 1H), 6.45-6.34 (m, 2H), 4.54 | |
| | | (d, J = 6.0 Hz, 2H), 3.03 (dd, J = 15.0, 5.6 Hz, 1H), 2.90 | |
| | | (dd, J = 14.9, 7.7 Hz, 1H), 1.46-1.21 (m, 2H), 0.79 (s, | |
| | | 1H), 0.52-0.40 (m, 1H), 0.41 (q, J = 2.8 Hz, 1H), 0.11- | |
| | | 0.03 (m, 1H). | |
| 57 | 6 | (300 MHz, Methanol-d4) δ 8.55 (s, 1H), 7.79 (d, J = 3.3 | 358.9 |
| | | Hz, 1H), 7.57 (d, J = 3.3 Hz, 1H), 6.64 (s, 1H), 4.96 (s, 2H), | |
| | | 3.77 (p, J = 6.6 Hz, 1H), 3.33-3.12 (m, 2H), 1.38 (d, J = | |
| | | 6.6 Hz, 3H). | |
| 58 | 6 | (400 MHz, Methanol-d4) δ 7.30 (dd, J = 5.1, 1.2 Hz, 1H), | 419.8 |
| | | 7.08 (dq, J = 3.4, 1.0 Hz, 1H), 6.97 (dd, J = 5.1, 3.5 Hz, | |
| | | 1H), 6.60 (s, 1H), 4.75 (d, J = 0.9 Hz, 2H), 4.50-4.24 (m, | |
| | | 2H), 3.55-3.41 (m, 1H), 3.10 (dd, J = 15.0, 6.1 Hz, 1H), | |
| | | 3.03-2.93 (m, 1H). | |
| 59 | 8 | (400 MHz, Methanol-d4) δ 7.28 (dd, J = 5.1, 1.2 Hz, 1H), | 395.9 |
| | | 7.06 (dq, J = 3.3, 1.1 Hz, 1H), 6.96 (dd, J = 5.1, 3.5 Hz, | |
| | | 1H), 6.49 (s, 1H), 4.75 (d, J = 1.0 Hz, 2H), 3.58 (dd, J = | |
| | | 10.9, 4.7 Hz, 1H), 3.47 (dd, J = 11.0, 6.3 Hz, 1H), 3.37- | |
| | | 3.32 (m, 1H), 3.08 (dd, J = 14.9, 6.0 Hz, 1H), 2.94 (dd, J = | |
| | | 15.0, 7.4 Hz, 1H), 2.44 (s, 3H). | |
| 60 | 8, 7 | (300 MHz, Methanol-d4) δ 8.52 (s, 1H), 7.35 (dd, J = 5.1, | 408.6 |
| | | 1.2 Hz, 1H), 7.15 (d, J = 1.2 Hz, 0H), 7.14 (s, 2H), 7.01 (dd, | |
| | | J = 5.1, 3.5 Hz, 1H), 4.85 (s, 2H), 3.86-3.59 (m, 3H), 3.33- | |
| | | 3.19 (m, 1H). | |
| 61 | 8, 7 | (400 MHz, Methanol-d4) δ 7.30 (dd, J = 5.1, 1.2 Hz, 1H), | 376.0 |
| | | 7.10-7.04 (m, 1H), 6.97 (dd, J = 5.1, 3.5 Hz, 1H), 6.56 (s, | |
| | | 1H), 4.75 (s, 2H), 3.87 (s, 1H), 3.47-3.38 (m, 2H), 3.35 | |
| | | (s, 3H), 3.32 (s, 1H), 3.12 (dd, J = 14.8, 5.9 Hz, 1H), 2.98 | |
| | | (dd, J = 14.8, 7.2 Hz, 1H). | |
| 62 | 8, 9 | (400 MHz, Methanol-d4) δ 7.77 (d, J = 3.3 Hz, 1H), 7.55 | 346.9 |
| | | (d, J = 3.3 Hz, 1H), 6.62 (s, 1H), 4.94 (s, 2H), 4.00 (s, 1H), | |
| | | 3.80 (h, J = 6.7 Hz, 1H), 3.30-3.20 (m, 2H), 1.36 (d, J = | |
| | | 6.7 Hz, 3H). | |
| 63 | 8, 9 | (400 MHz, Methanol-d4) δ 7.30 (dd, J = 5.1, 1.2 Hz, 1H), | 346.0 |
| | | 7.08 (dd, J = 3.5, 1.2 Hz, 1H), 6.97 (dd, J = 5.1, 3.5 Hz, | |
| | | 1H), 6.58 (s, 1H), 4.75 (s, 2H), 3.91 (s, 1H), 3.60 (h, J = 6.5 | |
| | | Hz, 1H), 3.20-3.02 (m, 2H), 1.25 (d, J = 6.5 Hz, 3H). | |
| 64 | 8, 9 | (400 MHz, Methanol-d4) δ 7.44 (dd, J = 1.9, 0.8 Hz, 1H), | 330.0 |
| | | 6.61 (s, 1H), 6.34 (ddd, J = 15.3, 3.2, 1.3 Hz, 2H), 4.54 (s, | |
| | | 2H), 3.85 (s, 1H), 3.42 (h, J = 6.5 Hz, 1H), 3.07-2.93 (m, | |
| | | 2H), 1.16 (d, J = 6.4 Hz, 3H). | |
| 65 | 8, 9 | (400 MHz, Methanol-d4) δ 7.77 (d, J = 3.3 Hz, 1H), 7.54 | 376.9 |
| | | (d, J = 3.4 Hz, 1H), 6.55 (s, 1H), 4.90 (s, 2H), 3.88 (s, 1H), | |
| | | 3.50-3.32 (m, 6H), 3.12 (dd, J = 14.8, 5.9 Hz, 1H), 3.04- | |
| | | 2.93 (m, 1H). | |
| 66 | 8, 9 | (400 MHz, Chloroform-d) δ 7.43-7.36 (m, 1H), 6.53 (s, | 359.9 |
| | | 1H), 6.36 (dd, J = 3.3, 1.9 Hz, 1H), 6.32 (d, J = 3.2 Hz, 1H), | |
| | | 5.20 (d, J = 6.7 Hz, 1H), 4.47 (d, J = 5.6 Hz, 2H), 3.49 (ddd, | |
| | | J = 12.1, 6.9, 5.0 Hz, 1H), 3.42 (dd, J = 9.2, 4.4 Hz, 1H), | |
| | | 3.39 (s, 1H), 3.37 (s, 3H), 3.31 (dd, J = 9.2, 6.5 Hz, 1H), | |
| | | 3.09 (dd, J = 14.8, 5.4 Hz, 1H), 2.97 (dd, J = 14.8, 7.9 Hz, 1H). | |
| 67 | 6 | H NMR (400 MHz, DMSO-d6): δ 7.66 (t, J = 6.0 Hz, 1H), | 394.3 |
| | | 7.39 (dd, J = 4.8 Hz, J = 1.2 Hz, 1H), 7.08 (dd, J = 3.2 Hz, | |
| | | J = 0.8 Hz, 1H), 6.98 (dd, J = 5.2 Hz, J = 3.6 Hz, 1H), 6.48 (s, | |
| | | 1H), 4.70 (d, J = 6.4 Hz, 2H), 3.46-3.31 (m, 1H), 3.00-2.90 | |
| | | (m, 1H), 2.82-2.77 (m, 1H), 2.07 (s, 3H), 2.00-1.70 (m, | |
| | | 2H), 1.02-0.92 (m, 2H), 0.71-0.57 (m, 2H). LCMS: m/z 394.31 | |
| 70 | 6 | (400 MHz, DMSO-d6): δ 7.66-7.63 (m, 1H), 7.39-7.37 | 392.3 |
| | | (m, 1H), 7.08 7.07 (m, 1H), 6.97-6.95 (m, 1H), 6.46 (s, | |
| | | 1H), 4.69 (d, J = 6.0 Hz, 2H), 4.53 (br s, 1H), 3.32-3.25 | |
| | | (obs, 3H), 2.87-2.75 (m, 3H), 2.67-2.63 (m, 1H), 2.13-2.09 | |
| | | (m, 1H), 2.05 (s, 3H), 1.89-1.82 (m, 1H), 1.00-0.96 (m, 1H). | |
| 73 | | (400 MHz, DMSO-d6): δ 7.65 (t, J = 6.0 Hz, 1H), 7.39 (dd, | 410.2 |
| | | J = 5.2 Hz, 1.2 Hz, 1H), 7.08 (dd, J = 3.6 Hz, 1.2 Hz, 1H), | |
| | | 6.97 (dd, J = 5.2 Hz, 3.6 Hz, 1H), 6.47 (s, 1H), 5.05 (t, J = | |
| | | 6.0 Hz, 1H), 4.70 (d, J = 6.0 Hz, 2H), 3.62-3.48 (m, 3H), | |
| | | 3.17-3.06 (m, 1H), 2.89-2.81 (m, 3H), 2.06 (s, 3H), 1.99- | |
| | | 1.88 (m, 1H), 1.58-1.46 (m, 1H). | |
| 77 | 10 | (300 MHz, Methanol-d4) δ 7.29 (t, J = 3.9 Hz, 1H), 7.07 | 424.0 |
| | | (s, 1H), 7.01-6.93 (m, 1H), 6.61-6.55 (m, 1H), 4.78- | |
| | | 4.71 (m, 2H), 3.44-3.34 (m, 1H), 3.22-3.08 (m, 1H), | |
| | | 3.04-2.94 (m, 1H), 2.47-2.29 (m, 3H). | |
| 85 | 8 | 400 MHz, Methanol-d4) δ 7.34-7.24 (m, 1H), 7.07 (t, J = | 459.8 |
| | | 2.4 Hz, 1H), 7.02-6.92 (m, 1H), 6.57 (t, J = 3.0 Hz, 1H), | |
| | | 4.75 (s, 2H), 3.66 (h, J = 5.6 Hz, 1H), 3.2-3.1 (m, 1H), | |
| | | 3.01-2.91 (m, 1H), 1.93 (dm, 2H), 1.12-0.92 (m, 2H), | |
| | | 0.74-0.49 (m, 2H). | |
| 86 | 8 | 400 MHz, Methanol-d4) δ 7.34-7.24 (m, 1H), 7.07 (t, J = | 459.8 |
| | | 2.4 Hz, 1H), 7.02-6.92 (m, 1H), 6.57 (t, J = 3.0 Hz, 1H), | |
| | | 4.75 (s, 2H), 3.66 (h, J = 5.6 Hz, 1H), 3.2-3.1 (m, 1H), | |
| | | 3.01-2.91 (m, 1H), 1.93 (dm, 2H), 1.12-0.92 (m, 2H), | |
| | | 0.74-0.49 (m, 2H). | |
| 87 | 8 | (400 MHz, Methanol-d4) δ 7.34-7.24 (m, 1H), 7.07 (t, | 459.8 |
| | | J = 2.4 Hz, 1H), 7.02-6.92 (m, 1H), 6.57 (t, J = 3.0 Hz, 1H), | |
| | | 4.75 (s, 2H), 3.66 (h, J = 5.6 Hz, 1H), 3.2-3.1 (m, 1H), | |
| | | 3.01-2.91 (m, 1H), 1.93 (dm, 2H), 1.12-0.92 (m, 2H), | |
| | | 0.74-0.49 (m, 2H). | |
| 88 | 10 | (400 MHz, Methanol-d4) δ 7.32 (dd, J = 5.1, 1.2 Hz, 1H), | 411.8 |
| | | 7.09 (dq, J = 3.3, 1.0 Hz, 1H), 6.98 (dd, J = 5.1, 3.5 Hz, | |
| | | 1H), 6.66 (d, J = 1.2 Hz, 1H), 4.66-4.59 (m, 1H), 3.53- | |
| | | 3.41 (m, 2H). | |
| 90 | 6 | (400 MHz, DMSO-d6) δ 8.23-8.11 (m, 1H), 7.78 (d, J = | 435.1 |
| | | 3.3 Hz, 1H), 7.65 (d, J = 3.3 Hz, 1H), 6.62 (s, 1H), 4.87 (d, | |
| | | J = 6.3 Hz, 2H), 4.54 (dq, J = 47.9, 6.2 Hz, 1H), 3.06 (dd, J = | |
| | | 15.0, 4.4 Hz, 1H), 2.81 (dd, J = 15.0, 8.9 Hz, 1H), 1.33 (dd, | |
| | | J = 24.8, 6.2 Hz, 3H). | |
| 91 | 6 | (400 MHz, Methanol-d4) δ 7.77 (d, J = 3.3 Hz, 1H), 7.56 | 435.0 |
| | | (d, J = 3.3 Hz, 1H), 6.65 (s, 1H), 4.95 (s, 2H), 4.82 (d, J = | |
| | | 6.0 Hz, 1H), 4.70 (q, J = 6.1 Hz, 1H), 3.69 (dq, J = 12.6, 6.1 | |
| | | Hz, 1H), 3.34 (d, J = 6.0 Hz, 1H), 3.22 (dd, J = 15.7, 7.2 Hz, | |
| | | 1H), 1.48 (dd, J = 24.7, 6.4 Hz, 3H). | |
| 92 | 6 | (300 MHz, Methanol-d4) δ 7.45-7.31 (m, 4H), 7.34- | 427.9 |
| | | 7.23 (m, 1H), 6.50 (s, 1H), 4.59 (s, 2H), 3.51 (ddt, J = 16.9, | |
| | | 8.8, 4.3 Hz, 1H), 3.24 (dd, J = 15.4, 4.7 Hz, 1H), 3.02 (dd, | |
| | | J = 15.3, 8.7 Hz, 1H), 1.43 (dd, J = 24.3, 6.4 Hz, 3H). | |
| 93 | 6 | (400 MHz, DMSO-d6) δ 7.94 (t, J = 6.3 Hz, 2H), 7.41- | 426.0 |
| | | 7.32 (m, 8H), 7.36-7.26 (m, 1H), 7.30-7.21 (m, 2H), | |
| | | 6.49 (s, 2H), 4.71 (dd, J = 6.4, 4.2 Hz, 1H), 4.63-4.52 (m, | |
| | | 5H), 3.05 (dd, J = 15.1, 5.6 Hz, 2H), 2.92 (dd, J = 15.1, 8.3 | |
| | | Hz, 2H), 1.36 (d, J = 6.3 Hz, 3H), 1.33 (s, 2H), 1.30 (d, J = | |
| | | 6.3 Hz, 3H). | |
| 94 | 6 | (300 MHz, Methanol-d4) δ 6.77 (t, J = 3.7 Hz, 1H), 6.65 | 449.9 |
| | | (s, 1H), 6.43 (dd, J = 4.0, 1.9 Hz, 1H), 4.65 (d, J = 2.7 Hz, | |
| | | 2H), 3.64 (ddt, J = 17.7, 8.8, 4.5 Hz, 1H), 3.34-3.23 (m, | |
| | | 1H), 3.08 (dd, J = 15.5, 8.8 Hz, 1H), 1.46 (dd, J = 24.3, 6.4 | |
| | | Hz, 3H). | |
| 95 | 6 | (400 MHz, DMSO-d6) δ 8.18 (s, 1H), 7.90 (t, J = 6.2 Hz, | 450.0 |
| | | 2H), 6.82 (t, J = 3.7 Hz, 2H), 6.66 (s, 2H), 6.57 (dd, J = 3.9, | |
| | | 2.1 Hz, 2H), 4.70 (tt, J = 6.5, 3.2 Hz, 1H), 4.62 (d, J = 2.7 | |
| | | Hz, 2H), 4.63-4.53 (m, 3H), 3.26 (dddd, J = 19.7, 9.0, | |
| | | 5.4, 3.9 Hz, 2H), 3.03 (dd, J = 15.0, 5.4 Hz, 2H), 2.90 (dd, | |
| | | J = 15.0, 8.4 Hz, 2H), 1.36 (d, J = 6.3 Hz, 3H), 1.30 (d, J = | |
| | | 6.2 Hz, 3H). | |
| 96 | 6 | (400 MHz, DMSO-d6) δ 7.92 (t, J = 6.1 Hz, 2H), 7.45 (dd, | 450.8 |
| | | J = 5.6, 4.1 Hz, 2H), 6.97 (d, J = 5.6 Hz, 2H), 6.61 (s, 2H), | |
| | | 4.63 (d, J = 6.1 Hz, 4H), 4.56 (p, J = 6.1 Hz, 1H), 4.44 (p, | |
| | | J = 6.1 Hz, 1H), 3.19 (tq, J = 10.8, 5.4, 4.7 Hz, 2H), 3.04 (dd, | |
| | | J = 14.9, 4.3 Hz, 2H), 2.78 (dd, J = 14.9, 9.0 Hz, 2H), 1.72 | |
| | | (s, 4H), 1.36 (d, J = 6.2 Hz, 3H), 1.30 (d, J = 6.2 Hz, 3H). | |
| 97 | 6 | (400 MHz, Methanol-d4) δ 7.29 (dd, J = 5.6, 4.0 Hz, 1H), | 449.9 |
| | | 6.84 (d, J = 5.5 Hz, 1H), 6.62 (s, 1H), 4.76-4.30 (m, 2H), | |
| | | 3.20-2.94 (m, 2H), 1.39 (dd, J = 24.4, 6.3 Hz, 3H). | |
| 98 | 6 | (400 MHz, Methanol-d4) δ 8.44 (s, 1H), 7.32 (dd, J = 5.1, | 443.9 |
| | | 1.2 Hz, 1H), 7.10 (dd, J = 3.5, 1.2 Hz, 1H), 6.99 (dd, J = | |
| | | 5.1, 3.5 Hz, 1H), 6.64 (s, 1H), 4.77 (dd, J = 7.8, 6.7 Hz, | |
| | | 1H), 4.69 (dd, J = 8.1, 6.5 Hz, 1H), 4.61 (t, J = 6.5 Hz, 1H), | |
| | | 4.31 (t, J = 6.5 Hz, 1H), 3.94 (dt, J = 9.9, 6.3 Hz, 1H), 3.30- | |
| | | 3.16 (m, 1H), 3.19-3.05 (m, 2H). | |
| 99 | 8 | (400 MHz, Methanol-d4) δ 7.30 (dd, J = 5.1, 1.2 Hz, 1H), | 455.0 |
| | | 7.07 (dd, J = 3.5, 1.2 Hz, 1H), 6.97 (dd, J = 5.1, 3.5 Hz, | |
| | | 1H), 6.60 (s, 1H), 4.76 (s, 2H), 3.94 (dd, J = 8.7, 7.6 Hz, | |
| | | 1H), 3.88 (td, J = 8.4, 3.8 Hz, 1H), 3.72 (td, J = 8.4, 7.0 Hz, | |
| | | 1H), 3.64-3.55 (m, 1H), 3.20-3.01 (m, 2H), 2.95 (dd, | |
| | | J = 14.8, 7.7 Hz, 1H), 2.25 (h, J = 8.1 Hz, 1H), 2.15-1.94 | |
| | | (m, 1H), 1.65 (dq, J = 12.2, 8.5 Hz, 1H). | |
| 100 | 8 | (400 MHz, Methanol-d4) δ 7.30 (dd, J = 5.0, 1.2 Hz, 1H), | 455.0 |
| | | 7.08 (dt, J = 3.5, 1.1 Hz, 1H), 6.97 (dd, J = 5.1, 3.5 Hz, 1H), | |
| | | 6.60 (s, 1H), 4.76 (d, J = 1.0 Hz, 2H), 3.97-3.82 (m, 2H), | |
| | | 3.71 (td, J = 8.5, 6.9 Hz, 1H), 3.50 (dd, J = 8.6, 7.4 Hz, 1H), | |
| | | 3.18 (td, J = 7.7, 5.0 Hz, 1H), 2.95 (qd, J = 15.0, 6.3 Hz, | |
| | | 2H), 2.26 (h, J = 7.8 Hz, 1H), 2.10 (dtd, J = 12.1, 8.1, 7.6, | |
| | | 3.8 Hz, 1H), 1.78 (dq, J = 12.3, 8.4 Hz, 1H). | |
| 103 | 8, 7 | (300 MHz, Methanol-d4) δ 8.44 (s, 1H), 7.35 (dd, J = 5.1, | 424.9 |
| | | 1.2 Hz, 1H), 7.15 (t, J = 1.1 Hz, 0H), 7.14 (s, 2H), 7.01 (dd, | |
| | | J = 5.1, 3.5 Hz, 1H), 5.00-4.92 (m, 0H), 4.85 (s, 1H), 4.78 | |
| | | (td, J = 6.4, 3.7 Hz, 0H), 3.73 (ddt, J = 18.5, 8.6, 4.4 Hz, | |
| | | 1H), 3.38 (d, J = 4.9 Hz, 0H), 3.16 (dd, J = 15.6, 8.8 Hz, | |
| | | 1H), 1.47 (dd, J = 24.3, 6.4 Hz, 3H). | |
| 105 | 6 | (400 MHz, Methanol-d4) δ 6.72 (ddd, J = 3.5, 2.7, 1.8 Hz, | 386.0 |
| | | 1H), 6.39 (dd, J = 3.9, 1.9 Hz, 1H), 4.68-4.45 (m, 3H), | |
| | | 3.28-3.20 (m, 1H), 3.05 (dd, J = 15.0, 4.5 Hz, 1H), 2.83 | |
| | | (dd, J = 15.0, 8.5 Hz, 1H), 2.16 (s, 3H), 1.37 (dd, J = 24.4, | |
| | | 6.3 Hz, 3H). | |
| 106 | 6 | (400 MHz, Methanol-d4) δ 7.77 (dd, J = 6.3, 3.2 Hz, 1H), | 369.0 |
| | | 7.53 (dd, J = 6.4, 3.3 Hz, 1H), 6.49 (d, J = 6.2 Hz, 1H), 4.60 | |
| | | (q, J = 6.1 Hz, 1H), 4.48 (q, J = 6.0 Hz, 1H), 3.38-3.21 (m, | |
| | | 9H), 3.09-3.02 (m, 1H), 2.83 (dd, J = 15.0, 8.3 Hz, 1H), | |
| | | 2.17 (d, J = 6.2 Hz, 3H), 1.37 (dt, J = 24.4, 6.2 Hz, 3H). | |
| 107 | 6 | (400 MHz, Methanol-d4) δ 7.42-7.28 (m, 1H), 7.27 (d, | 362.0 |
| | | J = 1.7 Hz, 0H), 6.37 (s, 0H), 4.56 (s, 0H), 4.52-4.42 (m, | |
| | | 0H), 3.24 (tt, J = 9.3, 4.6 Hz, 0H), 3.04 (dd, J = 15.0, 4.5 | |
| | | Hz, 0H), 2.82 (dd, J = 15.0, 8.6 Hz, 0H), 2.16 (s, 1H), 1.36 | |
| | | (dd, J = 24.4, 6.3 Hz, 1H). | |
| 108 | 6 | (400 MHz, DMSO-d6) δ 7.92 (t, J = 6.2 Hz, 2H), 6.83 (t, J = | 406.1 |
| | | 3.8 Hz, 2H), 6.68 (s, 2H), 6.58 (dd, J = 3.9, 2.0 Hz, 2H), | |
| | | 5.85 (s, 5H), 4.88-4.78 (m, 1H), 4.78-4.67 (m, 1H), | |
| | | 4.63 (dd, J = 6.4, 2.7 Hz, 4H), 3.51 (ddt, J = 16.4, 8.7, 4.5 | |
| | | Hz, 2H), 3.18 (dd, J = 15.5, 4.7 Hz, 2H), 2.96 (dd, J = 15.5, | |
| | | 8.7 Hz, 2H), 1.39 (d, J = 6.4 Hz, 3H), 1.33 (d, J = 6.3 Hz, 3H). | |
| 109 | 6 | (300 MHz, Methanol-d4) δ 7.33 (dd, J = 5.1, 1.2 Hz, 1H), | 388.1 |
| | | 7.10 (dd, J = 3.5, 1.2 Hz, 1H), 7.00 (dd, J = 5.1, 3.5 Hz, | |
| | | 1H), 6.63 (s, 1H), 4.83-4.56 (m, 3H), 3.47 (ddd, J = 16.4, | |
| | | 8.9, 4.5 Hz, 1H), 3.26-2.94 (m, 2H), 1.43 (dd, J = 24.3, | |
| | | 6.3 Hz, 3H). | |
| 110 | 6 | (400 MHz, DMSO-d6) δ 7.97 (t, J = 6.3 Hz, 2H), 7.41- | 382.1 |
| | | 7.32 (m, 6H), 7.36-7.30 (m, 2H), 7.26 (ddt, J = 8.6, 6.1, | |
| | | 1.9 Hz, 2H), 6.48 (s, 2H), 4.60 (p, J = 6.2 Hz, 1H), 4.54 (d, | |
| | | J = 6.2 Hz, 4H), 4.48 (p, J = 6.1 Hz, 1H), 3.27-3.14 (m, 1H), | |
| | | 3.06 (dd, J = 14.9, 4.3 Hz, 2H), 2.80 (dd, J = 15.0, 9.0 Hz, | |
| | | 2H), 1.37 (d, J = 6.2 Hz, 3H), 1.30 (d, J = 6.2 Hz, 3H). | |
| 111 | 6 | (400 MHz, Methanol-d4) δ 7.30 (dd, J = 5.1, 1.2 Hz, 1H), | 359.0 |
| | | 7.09 (dt, J = 3.4, 1.1 Hz, 1H), 7.02-6.95 (m, 2H), 4.82- | |
| | | 4.79 (m, 2H), 4.68-4.48 (m, 1H), 3.31 (p, J = 1.7 Hz, 8H), | |
| | | 3.09 (dd, J = 15.1, 4.4 Hz, 1H), 2.87 (dd, J = 15.0, 8.7 Hz, | |
| | | 1H), 2.20 (s, 3H), 1.38 (dd, J = 24.4, 6.3 Hz, 3H). | |
| 112 | 6, 7 | (400 MHz, Methanol-d4) δ 7.44 (dd, J = 2.0, 0.9 Hz, 1H), | 374.0 |
| | | 6.84 (s, 1H), 6.33 (ddd, J = 16.6, 3.2, 1.4 Hz, 2H), 4.56 (s, | |
| | | 2H), 3.56 (s, 1H), 3.46 (h, J = 6.5 Hz, 1H), 3.06-2.89 (m, | |
| | | 2H), 1.19 (d, J = 6.4 Hz, 3H). | |
| 113 | 6 | (400 MHz, DMSO-d6) δ 8.64 (d, J = 5.2 Hz, 2H), 8.24 (s, | 381.2 |
| | | 3H), 8.08 (t, J = 6.3 Hz, 1H), 7.58-7.52 (m, 2H), 6.55 (s, | |
| | | 1H), 4.84 (s, 1H), 4.73 (d, J = 6.3 Hz, 2H), 3.55 (dd, J = | |
| | | 10.4, 3.8 Hz, 1H), 3.43 (dd, J = 10.4, 5.8 Hz, 1H), 3.27- | |
| | | 3.11 (m, 2H). | |
| 114 | 6 | (400 MHz, Methanol-d4) δ 7.77 (d, J = 3.3 Hz, 1H), 7.56 | 435.0 |
| | | (d, J = 3.3 Hz, 1H), 6.65 (s, 1H), 4.95 (s, 2H), 4.82 (d, J = | |
| | | 6.0 Hz, 1H), 4.70 (q, J = 6.1 Hz, 1H), 3.69 (dq, J = 12.6, 6.1 | |
| | | Hz, 1H), 3.34 (d, J = 6.0 Hz, 1H), 3.22 (dd, J = 15.7, 7.2 Hz, | |
| | | 1H), 1.48 (dd, J = 24.7, 6.4 Hz, 3H). | |
| 115 | 6 | (400 MHz, Methanol-d4) δ 7.30 (dd, J = 5.1, 1.2 Hz, 1H), | 458.0 |
| | | 7.07 (dq, J = 3.3, 1.1 Hz, 1H), 6.97 (dd, J = 5.1, 3.5 Hz, | |
| | | 1H), 6.59 (s, 1H), 4.78-4.70 (m, 2H), 3.50 (dd, J = 7.2, | |
| | | 4.7 Hz, 1H), 3.47-3.38 (m, 2H), 3.30-3.26 (m, 1H), 3.04 | |
| | | (dd, J = 14.9, 5.7 Hz, 1H), 3.01-2.87 (m, 1H), 0.61-0.36 | |
| | | (m, 4H). | |
| 116 | 6 | (300 MHz, DMSO-d6) δ 7.30 (q, J = 1.9 Hz, 1H), 7.08 (d, | 484.0 |
| | | J = 3.4 Hz, 1H), 6.98 (q, J = 4.6, 3.1 Hz, 1H), 6.59 (d, J = 2.7 | |
| | | Hz, 1H), 4.75 (d, J = 2.3 Hz, 2H), 4.61 (s, 2H), 4.00 (qd, J = | |
| | | 9.6, 5.3 Hz, 2H), 3.54 (p, J = 6.0 Hz, 1H), 3.18-3.07 (m, | |
| | | 1H), 3.00 (dd, J = 15.2, 7.8 Hz, 1H) | |
| 117 | 12 | (400 MHz, DMSO-d6) δ 7.95 (t, J = 6.2 Hz, 1H), 7.42 (dd, | 467.7 |
| | | J = 5.1, 1.2 Hz, 1H), 7.11 (dd, J = 3.4, 1.2 Hz, 1H), 6.99 (dd, | |
| | | J = 5.1, 3.4 Hz, 1H), 6.95-6.50 (m, 2H), 5.35 (s, 2H), 4.74 | |
| | | (d, J = 6.1 Hz, 2H), 3.87 (qd, J = 10.3, 5.4 Hz, 2H), 3.58 (p, | |
| | | J = 5.8 Hz, 1H), 3.15-2.96 (m, 2H). | |
| 118 | 6 | (400 MHz, Methanol-d4) δ 7.85 (dd, J = 2.0, 0.9 Hz, 1H), | 418.1 |
| | | 7.68 (s, 1H), 6.75 (ddd, J = 11.4, 3.2, 1.3 Hz, 1H), 6.43 (p, | |
| | | J = 6.2 Hz, 2H), 4.76 (s, 1H), 4.52 (dt, J = 16.9, 6.2 Hz, 2H), | |
| | | 3.15 (dd, J = 15.4, 5.7 Hz, 1H), 2.91 (dd, J = 15.4, 7.5 Hz, | |
| | | 1H), 1.34 (dd, J = 24.5, 6.3 Hz, 3H). | |
| 119 | 6 | (400 MHz, DMSO-d6) δ 8.54-8.48 (m, 2H), 8.00 (t, J = | 428.8 |
| | | 6.4 Hz, 1H), 7.38-7.32 (m, 2H), 6.46 (s, 1H), 4.57 (dd, J = | |
| | | 12.9, 6.2 Hz, 2H), 4.45 (p, J = 6.2 Hz, 1H), 3.16 (dq, J = | |
| | | 11.0, 5.7 Hz, 2H), 3.05 (dd, J = 14.8, 4.3 Hz, 1H), 2.78 (dd, | |
| | | J = 14.9, 9.0 Hz, 1H), 1.30-1.36 (d, J = 6.2 Hz, 3H). | |
| 120 | 6, 9 | (300 MHz, DMSO-d6) δ 7.80 (t, J = 6.1 Hz, 1H), 7.60 (dd, | 397.9 |
| | | J = 1.9, 0.9 Hz, 1H), 6.64 (s, 1H), 6.44-6.34 (m, 2H), 4.56- | |
| | | 4.44 (m, 3H), 3.52 (tt, J = 7.8, 5.1 Hz, 1H), 3.06 (dd, J = | |
| | | 14.8, 5.3 Hz, 1H), 2.96 (dd, J = 14.8, 7.9 Hz, 1H), 2.51- | |
| | | 2.30 (m, 1H). | |
| 121 | 6, 9 | (300 MHz, Methanol-d4) δ 7.32 (dt, J = 5.1, 1.4 Hz, 1H), | 413.9 |
| | | 7.09 (dt, J = 3.5, 1.2 Hz, 1H), 6.99 (ddd, J = 4.9, 3.4, 1.2 | |
| | | Hz, 1H), 6.62-6.55 (m, 1H), 4.76 (d, J = 1.9 Hz, 2H), 3.92 | |
| | | (d, J = 1.0 Hz, 1H), 3.69 (tt, J = 7.6, 5.2 Hz, 1H), 3.19 (dd, | |
| | | J = 14.9, 5.5 Hz, 1H), 3.09 (dd, J = 14.9, 7.6 Hz, 1H), 2.60- | |
| | | 2.24 (m, 2H). | |
| 122 | 6, 9 | (400 MHz, Methanol-d4) δ 7.45 (dd, J = 2.0, 0.8 Hz, 2H), | 361.9 |
| | | 6.67-6.61 (m, 2H), 6.39-6.29 (m, 4H), 4.76 (d, J = 5.4 | |
| | | Hz, 1H), 4.65 (dd, J = 6.5, 4.5 Hz, 1H), 4.56 (d, J = 17.6 Hz, | |
| | | 1H), 4.54 (s, 3H), 3.92 (s, 2H), 3.54 (ddt, J = 17.3, 9.1, 4.6 | |
| | | Hz, 2H), 3.27 (d, J = 4.9 Hz, 1H), 3.05 (dd, J = 15.3, 8.7 Hz, | |
| | | 2H), 1.41 (dd, J = 24.3, 6.4 Hz, 6H). | |
| 123 | 6, 9 | (400 MHz, Methanol-d4) δ 7.34-7.27 (m, 1H), 7.08 (dt, | 377.9 |
| | | J = 3.5, 1.1 Hz, 1H), 6.98 (td, J = 3.6, 1.9 Hz, 1H), 6.63- | |
| | | 6.56 (m, 1H), 4.82-4.63 (m, 3H), 3.94 (s, 1H), 3.60 (ddt, | |
| | | J = 17.5, 8.8, 4.5 Hz, 1H), 3.09 (dd, J = 15.4, 8.7 Hz, 1H), | |
| | | 1.42 (dd, J = 24.3, 6.4 Hz, 3H). | |
| 124 | 8 | (400 MHz, Methanol-d4) δ 7.91 (d, J = 0.9 Hz, 1H), 7.15 | 387.1 |
| | | (d, J = 0.9 Hz, 1H), 6.69 (s, 1H), 4.76 (s, 2H), 3.79 (q, J = | |
| | | 6.6 Hz, 1H), 3.31-3.15 (m, 2H), 1.38 (d, J = 6.7 Hz, 3H). | |
| 125 | 8 | (300 MHz, Methanol-d4) δ 7.34 (dd, J = 5.1, 1.2 Hz, 1H), | 431.9 |
| | | 7.11 (dt, J = 3.3, 1.0 Hz, 1H), 7.00 (dd, J = 5.1, 3.5 Hz, 1H), | |
| | | 6.66 (s, 1H), 4.79 (s, 2H), 4.03-3.89 (m, 1H), 3.55 (ddd, | |
| | | J = 8.8, 5.1, 3.7 Hz, 1H), 3.28 (dd, J = 15.5, 4.9 Hz, 1H), 3.11 | |
| | | (dd, J = 15.6, 8.8 Hz, 1H), 1.29 (d, J = 6.5 Hz, 3H). | |
| 126 | 8 | (400 MHz, Methanol-d4) δ 7.32 (dd, J = 5.0, 1.2 Hz, 1H), | 431.9 |
| | | 7.09 (dd, J = 3.5, 1.2 Hz, 1H), 6.98 (dd, J = 5.1, 3.4 Hz, | |
| | | 1H), 6.64 (s, 1H), 4.77 (s, 2H), 3.77 (p, J = 6.3 Hz, 1H), | |
| | | 3.37 (dt, J = 7.9, 5.5 Hz, 1H), 3.27 (dd, J = 15.4, 5.4 Hz, | |
| | | 1H), 3.11 (dd, J = 15.5, 7.9 Hz, 1H), 1.29 (d, J = 6.3 Hz, 3H). | |
| 131 | 6 | (400 MHz, Methanol-d4) δ 8.53 (s, 1H), 7.31 (dd, J = 5.1, | 431.8 |
| | | 1.2 Hz, 1H), 7.09 (dd, J = 3.5, 1.2 Hz, 1H), 6.98 (dd, J = | |
| | | 5.1, 3.4 Hz, 1H), 6.63 (s, 1H), 4.77 (s, 2H), 3.85-3.70 (m, | |
| | | 3H), 3.27-3.17 (m, 2H), 1.86 (dtd, J = 10.8, 7.3, 6.6, 4.4 | |
| | | Hz, 2H). | |
| 132 | 10 | (400 MHz, Methanol-d4) δ 7.31 (dd, J = 5.1, 1.2 Hz, 1H), | 426.0 |
| | | 7.08 (dt, J = 3.5, 1.1 Hz, 1H), 6.98 (dd, J = 5.1, 3.5 Hz, 1H), | |
| | | 6.61 (s, 1H), 4.80-4.71 (m, 2H), 4.11 (td, J = 7.1, 2.2 Hz, | |
| | | 1H), 2.94 (d, J = 2.2 Hz, 1H), 2.60 (s, 3H). | |
| 133 | 8 | (400 MHz, Methanol-d4) δ 7.30 (dd, J = 5.1, 1.2 Hz, 1H), | 476.0 |
| | | 7.08 (dq, J = 3.4, 1.1 Hz, 1H), 6.97 (dd, J = 5.1, 3.5 Hz, | |
| | | 1H), 6.59 (d, J = 0.9 Hz, 1H), 4.76 (s, 2H), 3.24-3.12 (m, | |
| | | 1H), 3.05-2.89 (m, 2H), 2.72-2.33 (m, 3H), 2.33-2.05 | |
| | | (m, 2H). | |
| 134 | 8 | (400 MHz, Methanol-d4) δ 7.30 (dd, J = 5.0, 1.2 Hz, 1H), | 427.0 |
| | | 7.06 (dt, J = 3.3, 1.1 Hz, 1H), 6.97 (dd, J = 5.1, 3.5 Hz, 1H), | |
| | | 6.59 (s, 1H), 4.76 (s, 2H), 3.20-3.01 (m, 2H), 2.46 (dt, J = | |
| | | 8.9, 6.8 Hz, 1H), 0.85 (dddd, J = 13.0, 9.6, 8.2, 4.9 Hz, | |
| | | 1H), 0.51 (dt, J = 10.0, 5.1 Hz, 1H), 0.47-0.36 (m, 1H), | |
| | | 0.26 (dq, J = 9.8, 4.9 Hz, 1H), 0.04-−0.04 (m, 3H). | |
| 139 | 8, 9 | (400 MHz, Methanol-d4) δ 7.30 (dd, J = 5.1, 1.2 Hz, 1H), | 422.0 |
| | | 7.07 (dq, J = 3.3, 1.1 Hz, 1H), 6.97 (dd, J = 5.1, 3.5 Hz, | |
| | | 1H), 6.58 (s, 1H), 4.75 (d, J = 1.0 Hz, 2H), 3.90 (s, 1H), | |
| | | 3.17 (td, J = 7.3, 5.3 Hz, 1H), 3.09-2.94 (m, 2H), 2.67- | |
| | | 2.55 (m, 1H), 2.46-2.35 (m, 1H), 2.31-2.17 (m, 1H), | |
| | | 2.15-2.08 (m, 1H). | |
| 140 | 6 | (400 MHz, DMSO-d6) δ 7.93 (dt, J = 9.0, 4.3 Hz, 2H), 7.46 | 406.1 |
| | | (t, J = 5.0 Hz, 2H), 6.98 (d, J = 5.5 Hz, 2H), 6.64 (s, 2H), | |
| | | 4.80-4.73 (m, 1H), 4.65 (d, J = 6.1 Hz, 5H), 3.45 (s, 3H), | |
| | | 3.38 (s, 3H), 3.14 (dd, J = 15.5, 4.5 Hz, 2H), 2.91 (ddd, J = | |
| | | 15.4, 9.0, 2.4 Hz, 2H), 2.51 (s, 1H), 1.38 (d, J = 6.2 Hz, | |
| | | 3H), 1.32 (d, J = 6.2 Hz, 3H). | |
| 142 | 6 | (400 MHz, Methanol-d4) δ 7.27 (dd, J = 5.6, 4.0 Hz, 1H), | 386.0 |
| | | 6.83 (dd, J = 5.6, 0.7 Hz, 1H), 6.52 (s, 1H), 4.66 (d, J = 1.2 | |
| | | Hz, 2H), 4.64-4.46 (m, 1H), 3.28-3.24 (m, 1H), 3.04 | |
| | | (dd, J = 15.0, 4.5 Hz, 1H), 2.82 (dd, J = 15.0, 8.6 Hz, 1H), | |
| | | 2.16 (s, 3H), 1.45-1.33 (m, 2H). | |
| 144 | 8 | (400 MHz, Methanol-d4) δ 7.35-7.25 (m, 1H), 7.12- | 457.9 |
| | | 7.04 (m, 1H), 7.02-6.92 (m, 1H), 6.59 (s, 1H), 4.75 (s, | |
| | | 2H), 3.34 (s, 3H), 3.29-3.17 (m, 2H), 3.11-2.93 (m, | |
| | | 1H), 0.90-0.72 (m, 2H), 0.64-0.54 (m, 2H). | |
| 145 | 14 | (300 MHz, Methanol-d4) δ 7.30 (m, 1H), 7.08 (m, 1H), | 433.8 |
| | | 6.97 (m, 1H), 6.60 (d, J = 2.4 Hz, 1H), 4.76 (s, 2H), 4.72- | |
| | | 4.49 (m, 2H), 3.54 (m, 1H), 3.08 (m, 2H), 2.04-1.75 (m, 2H). | |
| 146 | 8 | (400 MHz, Methanol-d4) δ 7.34-7.24 (m, 1H), 7.12- | 499.8 |
| | | 7.02 (m, 1H), 7.02-6.92 (m, 1H), 6.59 (d, J = 5.7 Hz, 1H), | |
| | | 4.75 (d, J = 3.3 Hz, 2H), 4.22-4.12 (m, 2H), 3.44-3.34 | |
| | | (m, 1H), 3.08-2.91 (m, 2H), 1.89-1.79 (m, 2H). | |
| 147 | 8 | 400 MHz, Methanol-d4) δ 7.35-7.25 (m, 1H), 7.12- | 444 |
| | | 7.02 (m, 1H), 7.02-6.92 (m, 1H), 6.59 (s, 1H), 4.75 (d, J = | |
| | | 1.0 Hz, 2H), 3.60-3.44 (m, 2H), 3.44-3.36 (m, 1H), 3.30- | |
| | | 3.29 (m, 3H), 3.06-2.90 (m, 2H), 1.86-1.57 (m, 2H). | |
| 148 | 6 | (300 MHz, Methanol-d4) δ 7.33 (dd, J = 5.1, 1.2 Hz, 1H), | 388.1 |
| | | 7.10 (dd, J = 3.5, 1.2 Hz, 1H), 7.00 (dd, J = 5.1, 3.5 Hz, | |
| | | 1H), 6.63 (s, 1H), 4.83-4.56 (m, 3H), 3.47 (ddd, J = 16.4, | |
| | | 8.9, 4.5 Hz, 1H), 3.26-2.94 (m, 2H), 1.43 (dd, J = 24.3, | |
| | | 6.3 Hz, 3H). | |
| 149 | 6 | (300 MHz, DMSO-d6) δ 8.56 (d, J = 1.8 Hz, 1H), 8.38 (d, | 402.9 |
| | | J = 4.9 Hz, 1H), 7.96 (t, J = 6.3 Hz, 1H), 7.37 (dd, J = 6.5, 4.9 | |
| | | Hz, 1H), 6.57 (s, 1H), 4.68 (d, J = 6.2 Hz, 2H), 4.58 (p, J = | |
| | | 6.2 Hz, 0.5H), 4.42 (p, J = 6.2 Hz, 0.5H), 3.18-2.97 (m, | |
| | | 2H), 2.78 (dd, J = 14.8, 8.9 Hz, 1H), 1.68 (s,1H), 1.33 (dd, | |
| | | J = 24.8, 6.2 Hz, 3H). | |
| 150 | 6 | (300 MHz, DMSO-d6) 87.84 (t, J = 6.1 Hz, 1H), 7.64- | 371.9 |
| | | 7.57 (m, 1H), 6.67 (s, 1H), 6.45-6.35 (m, 2H), 4.69 (p, J = | |
| | | 6.2 Hz, 1H), 4.53 (dd, J = 6.1, 2.8 Hz, 2H), 3.38-3.20 (m, | |
| | | 6H), 3.09 (dd, J = 15.1, 4.4 Hz, 1H), 2.85 (dd, J = 15.2, 8.8 | |
| | | Hz, 1H), 1.39 (d, J = 6.2 Hz, 3H), 1.37 (d, J = 6.2 Hz, 3H). | |
| 151 | 8, 9 | (400 MHz, Methanol-d4) δ 7.29 (dd, J = 5.1, 1.2 Hz, 1H), | 422.0 |
| | | 7.07 (dd, J = 3.5, 1.2 Hz, 1H), 6.97 (dd, J = 5.1, 3.5 Hz, | |
| | | 1H), 6.79 (s, 1H), 4.77 (s, 2H), 3.53 (s, 1H), 3.44-3.34 | |
| | | (m, 5H), 3.28 (d, J = 5.8 Hz, 1H), 3.07 (dd, J = 14.8, 5.9 Hz, | |
| | | 1H), 2.94 (dd, J = 14.9, 7.2 Hz, 1H). | |
| 152 | 8 | (300 MHz, DMSO-d6) δ 7.89 (t, J = 6.2 Hz, 1H), 7.44- | 445.9 |
| | | 7.28 (m, 2H), 7.20 (dtd, J = 14.8, 7.9, 7.4, 1.3 Hz, 2H), | |
| | | 6.53 (s, 1H), 4.58 (t, J = 6.0 Hz, 2H), 3.25-3.10 (m, 1H), | |
| | | 3.04 (dd, J = 14.8, 4.2 Hz, 1H), 2.78 (dd, J = 14.9, 9.0 Hz, | |
| | | 1H), 1.77 (s, 2H), 1.33 (dd, J = 24.7, 6.2 Hz, 3H). | |
| 153 | 8 | (300 MHz, DMSO-d6) δ 7.78 (s, 1H), 7.44 (q, J = 7.3 Hz, | 463.9 |
| | | 1H), 7.15 (t, J = 8.0 Hz, 2H), 6.67 (s, 1H), 4.70-4.29 (m, | |
| | | 3H), 3.17 (d, J = 5.8 Hz, 1H), 3.03 (dd, J = 14.8, 4.3 Hz, | |
| | | 1H), 2.76 (dd, J = 14.9, 8.9 Hz, 1H), 1.63 (s, 2H), 1.32 (dd, | |
| | | J = 24.8, 6.2 Hz, 3H). | |
| 154 | 12 | (300 MHz, Methanol-d4) δ 8.81 (s, 1H), 7.29-7.19 (m, | 480.0 |
| | | 1H), 7.04-6.93 (m, 2H), 3.46-3.35 (m, 1H), 3.20-3.01 | |
| | | (m, 2H), 2.88-2.76 (m, 1H), 2.59 (s, 3H), 2.47-2.37 (m, | |
| | | 1H), 2.03-1.93 (m, 1H), 1.79-1.69 (m, 1H), 1.23 (d, J = | |
| | | 6.4 Hz, 3H). | |
| 155 | 8 | (300 MHz, Methanol-d4) δ 7.51-7.42 (m, 2H), 6.66 (s, | 410.8 |
| | | 2H), 6.72-6.58 (m, 0H), 6.44-6.29 (m, 5H), 4.56 (s, | |
| | | 4H), 4.55 (dd, J = 11.5, 9.0 Hz, 1H), 3.89 (t, J = 7.2 Hz, | |
| | | 2H), 3.11 (dd, J = 8.5, 6.7 Hz, 4H), 2.21 (tdd, J = 7.8, 6.4, | |
| | | 1.2 Hz, 4H). | |
| 157 | 8 | (400 MHz, Methanol-d4) δ 7.31 (dd, J = 5.1, 1.2 Hz, 1H), | 432.9 |
| | | 7.09 (dq, J = 3.3, 1.0 Hz, 1H), 6.98 (dd, J = 5.1, 3.5 Hz, | |
| | | 1H), 6.65 (s, 1H), 4.78 (d, J = 1.0 Hz, 2H), 3.84 (qd, J = 6.9, | |
| | | 3.4 Hz, 1H), 3.63 (dd, J = 10.5, 3.5 Hz, 1H), 3.47 (dd, J = | |
| | | 10.5, 5.7 Hz, 1H), 3.36-3.32 (m, 1H), 3.24 (dd, J = 15.3, | |
| | | 6.8 Hz, 1H). | |
| 158 | 8, 7 | | |
| 160 | 8, 7 | (400 MHz, Methanol-d4) δ 7.29 (dd, J = 5.1, 1.2 Hz, 1H), | 404.0 |
| | | 7.06 (d, J = 3.5 Hz, 1H), 6.97 (dd, J = 5.1, 3.5 Hz, 1H), 6.68 | |
| | | (s, 1H), 4.75 (s, 2H), 3.48-3.39 (m, 1H), 2.98 (t, J = 6.1 | |
| | | Hz, 2H), 2.03 (s, 3H), 1.18 (d, J = 6.4 Hz, 3H). | |
| 162 | 8, 7, 9 | (400 MHz, Methanol-d4) δ 7.29 (dd, J = 5.1, 1.2 Hz, 1H), | 336.0 |
| | | 7.07 (dq, J = 3.4, 1.1 Hz, 1H), 6.97 (dd, J = 5.1, 3.5 Hz, | |
| | | 1H), 6.77 (s, 1H), 4.76 (d, J = 1.0 Hz, 2H), 3.85 (s, 1H), | |
| | | 3.52-3.39 (m, 2H), 3.09-2.95 (m, 2H), 1.17 (d, J = 6.4 | |
| | | Hz, 3H). | |
| 164 | 8, 7 | (300 MHz, Methanol-d4) δ 7.29 (dd, J = 5.1, 1.2 Hz, 1H), | 391.9 |
| | | 7.10 (dq, J = 3.4, 1.1 Hz, 1H), 6.98 (dd, J = 5.1, 3.5 Hz, | |
| | | 1H), 6.89-6.74 (m, 2H), 5.98 (dd, J = 17.6, 1.1 Hz, 1H), | |
| | | 5.41 (dd, J = 11.0, 1.1 Hz, 1H), 4.84-4.77 (m, 2H), 3.43 | |
| | | (h, J = 6.5 Hz, 1H), 3.06-2.87 (m, 2H), 1.18 (d, J = 6.4 Hz, 3H). | |
| 167 | | (300 MHz, Methanol-d4) δ 7.50 (s, 1H), 7.34 (dd, J = 5.1, | 409.8 |
| | | 1.1 Hz, 1H), 7.15 (d, J = 3.2 Hz, 1H), 7.00 (dd, J = 5.1, 3.5 | |
| | | Hz, 1H), 3.89 (d, J = 7.4 Hz, 1H), 3.27 (q, J = 8.5, 7.2 Hz, | |
| | | 2H), 1.44 (d, J = 6.5 Hz, 3H). | |
| 169 | 8 | (400 MHz, Methanol-d4) δ 8.53 (s, 1H), 7.31 (dd, J = 5.1, | 416.0 |
| | | 1.2 Hz, 1H), 7.11-7.06 (m, 1H), 6.98 (dd, J = 5.1, 3.5 Hz, | |
| | | 1H), 6.64 (s, 1H), 4.77 (s, 2H), 3.46 (q, J = 6.4 Hz, 1H), | |
| | | 3.19 (dd, J = 15.3, 6.0 Hz, 1H), 3.11 (dd, J = 15.3, 7.1 Hz, | |
| | | 1H), 1.65 (dh, J = 21.2, 6.9 Hz, 2H), 1.05 (t, J = 7.5 Hz, 3H). | |
| 170 | 13 | (400 MHz, Methanol-d4) δ 7.29 (d, J = 5.1 Hz, 1H), 7.06 | 425.8 |
| | | (d, J = 3.5 Hz, 1H), 7.00-6.91 (m, 1H), 6.57 (s, 1H), 3.58 | |
| | | (q, J = 8.3 Hz, 1H), 3.21 (q, J = 9.0 Hz, 1H), 2.16 (dp, J = | |
| | | 12.8, 7.1, 6.4 Hz, 2H), 2.05-1.95 (m, 1H), 1.89 (d, J = 6.7 | |
| | | Hz, 2H), 1.58 (dq, J = 12.6, 8.7 Hz, 1H). | |
| 172 | 13 | (400 MHz, Methanol-d4) δ 8.51 (d, J = 5.4 Hz, 1H), 7.31 | 425.8 |
| | | (d, J = 5.2 Hz, 1H), 7.07 (d, J = 3.5 Hz, 1H), 7.00-6.94 (m, | |
| | | 1H), 6.61 (d, J = 21.8 Hz, 1H), 3.89 (q, J = 8.3 Hz, 1H), | |
| | | 3.57-3.45 (m, 1H), 2.41-2.23 (m, 2H), 2.01 (td, J = | |
| | | 14.6, 12.6, 7.2 Hz, 3H), 1.87-1.72 (m, 1H). | |
| 174 | 13 | (400 MHz, Methanol-d4) δ 7.30 (dd, J = 5.2, 1.2 Hz, 1H), | 439.8 |
| | | 7.07 (d, J = 3.5 Hz, 1H), 6.97 (dd, J = 5.1, 3.5 Hz, 1H), 6.59 | |
| | | (d, J = 2.8 Hz, 1H), 3.13 (t, J = 5.4 Hz, 1H), 2.89 (d, J = 10.7 | |
| | | Hz, 1H), 2.05 (d, J = 12.6 Hz, 1H), 1.86 (d, J = 8.2 Hz, 3H), | |
| | | 1.73 (t, J = 12.3 Hz, 1H), 1.39 (dt, J = 37.5, 12.7 Hz, 3H). | |
| 176 | 13 | (400 MHz, Methanol-d4) δ 7.30 (d, J = 5.1 Hz, 1H), 7.07 | 349.8 |
| | | (d, J = 3.5 Hz, 1H), 6.97 (dd, J = 5.0, 3.5 Hz, 1H), 6.58 (s, | |
| | | 1H), 3.14 (td, J = 10.8, 3.9 Hz, 1H), 2.90-2.80 (m, 1H), | |
| | | 2.12-2.00 (m, 1H), 1.92-1.72 (m, 4H), 1.39 (ddt, J = | |
| | | 35.2, 23.3, 12.0 Hz, 3H) | |
| 178 | 8, 7, 9 | (400 MHz, Methanol-d4) δ 7.31 (d, J = 1.2 Hz, 1H), 7.08 | 412.0 |
| | | (d, J = 1.1 Hz, 1H), 6.98 (dd, J = 5.1, 3.5 Hz, 2H), 6.80 (s, | |
| | | 2H), 4.78 (s, 4H), 4.60 (s, 1H), 3.93 (s, 2H), 3.53 (s, 2H), | |
| | | 3.41-3.30 (m, 44H), 3.18-3.05 (m, 4H), 2.62 (dtd, J = | |
| | | 18.0, 12.9, 6.5 Hz, 4H), 2.46 (dt, J = 26.4, 12.7 Hz, 2H), | |
| | | 2.25 (ddd, J = 38.8, 17.3, 8.7 Hz, 4H). | |
| 180 | 8, 9 | (400 MHz, Methanol-d4) δ 7.29 (dd, J = 5.1, 1.2 Hz, 1H), | 372.0 |
| | | 7.06 (dt, J = 3.5, 1.1 Hz, 1H), 6.97 (dd, J = 5.1, 3.5 Hz, 1H), | |
| | | 6.56 (s, 1H), 4.75 (s, 2H), 3.85 (s, 1H), 3.23-3.09 (m, | |
| | | 2H), 2.48 (dt, J = 9.4, 6.7 Hz, 1H), 0.93-0.81 (m, 1H), | |
| | | 0.51 (tdd, J = 8.2, 5.6, 4.3 Hz, 1H), 0.44-0.38 (m, 1H), | |
| | | 0.27 (dq, J = 9.7, 4.9 Hz, 1H), 0.06-0.02 (m, 2H). | |
| 181 | 8, 7, 9 | (400 MHz, Methanol-d4) δ 7.29 (dd, J = 5.1, 1.2 Hz, 1H), | 362.0 |
| | | 7.06 (dq, J = 3.4, 1.0 Hz, 1H), 6.96 (dd, J = 5.1, 3.5 Hz, | |
| | | 1H), 6.77 (s, 1H), 4.76 (s, 2H), 3.84 (s, 1H), 3.50 (s, 1H), | |
| | | 3.26-3.11 (m, 2H), 2.49 (dt, J = 9.3, 6.7 Hz, 1H), 0.86 | |
| | | (dtt, J = 9.6, 8.2, 4.9 Hz, 1H), 0.50 (tdd, J = 8.2, 5.6, 4.3 | |
| | | Hz, 1H), 0.46-0.40 (m, 1H), 0.27 (dt, J = 9.6, 4.8 Hz, 1H), | |
| | | 0.07-−0.04 (m, 2H). | |
| 182 | 8 | (300 MHz, Methanol-d4) δ 7.30 (dd, J = 5.1, 1.2 Hz, 2H), | 481.8 |
| | | 7.11-7.04 (m, 2H), 6.97 (dd, J = 5.1, 3.6 Hz, 2H), 6.60 (s, | |
| | | 2H), 5.96 (t, J = 3.9 Hz, 1H), 4.76 (s, 3H), 3.81-3.49 (m, | |
| | | 4H), 3.18 (dd, J = 15.0, 6.1 Hz, 2H), 3.06 (dd, J = 15.1, 6.6 | |
| | | Hz, 2H). | |
| 183 | 8 | (400 MHz, Methanol-d4) δ 7.28 (d, J = 5.1 Hz, 1H), 7.06 | 477.7 |
| | | (d, J = 3.6 Hz, 1H), 7.01-6.91 (m, 1H), 6.62-6.52 (m, | |
| | | 1H), 4.74 (s, 2H), 3.41-3.31 (m, 1H), 3.11-3.01 (m, | |
| | | 1H), 2.96-2.86 (m, 1H), 1.80-1.55 (m, 3H), 1.53-1.43 | |
| | | (m, 1H), 1.11-0.96 (m, 1H). | |
| 184 | 8 | 400 MHz, Methanol-d4) δ 7.28 (d, J = 5.1 Hz, 1H), 7.06 | 477.8 |
| | | (d, J = 3.6 Hz, 1H), 7.01-6.91 (m, 1H), 6.62-6.52 (m, | |
| | | 1H), 4.74 (s, 2H), 3.41-3.31 (m, 1H), 3.11-3.01 (m, | |
| | | 1H), 2.96-2.86 (m, 1H), 1.80-1.55 (m, 3H), 1.53-1.43 | |
| | | (m, 1H), 1.11-0.96 (m, 1H). | |
| 187 | 11 | (400 MHz, Methanol-d4) δ 7.31 (dd, J = 5.1, 1.2 Hz, 1H), | 436.0 |
| | | 7.09 (dt, J = 3.3, 1.1 Hz, 1H), 6.98 (dd, J = 5.1, 3.5 Hz, 1H), | |
| | | 6.69 (s, 1H), 4.77 (s, 2H), 3.65 (dt, J = 12.3, 6.1 Hz, 1H), | |
| | | 1.21 (d, J = 6.8 Hz, 3H). | |
| 188 | 12 | (400 MHz, Methanol-d4) δ 8.47 (s, 1H), 7.31 (d, J = 5.1 | 421.9 |
| | | Hz, 1H), 7.09 (d, J = 3.5 Hz, 1H), 6.98 (dd, J = 5.1, 3.6 Hz, | |
| | | 1H), 6.73-6.28 (m, 2H), 4.76 (s, 2H), 4.08 (dd, J = 10.8, | |
| | | 3.9 Hz, 1H), 3.95 (dd, J = 10.7, 5.6 Hz, 1H), 3.81 (q, J = 6.0 | |
| | | Hz, 1H), 3.27 (dd, J = 15.4, 6.3 Hz, 1H), 3.18 (dd, J = 15.4, | |
| | | 7.3 Hz, 1H). | |
| 189 | 11 | (400 MHz, Methanol-d4) δ 8.27 (s, 1H), 7.31 (dd, J = 5.1, | 392.0 |
| | | 1.2 Hz, 1H), 7.09 (dt, J = 3.4, 1.1 Hz, 1H), 6.98 (dd, J = 5.1, | |
| | | 3.5 Hz, 1H), 6.71 (s, 1H), 4.77 (s, 2H),3.83 (td, J = 12.4, | |
| | | 6.5 Hz, 1H), 1.28 (d, J = 6.7 Hz, 3H). | |
| 190 | 11 | (400 MHz, Methanol-d4) δ 7.32 (dd, J = 5.1, 1.2 Hz, 1H), | 407.9 |
| | | 7.10 (dd, J = 3.5, 1.1 Hz, 1H), 6.98 (dd, J = 5.1, 3.5 Hz, | |
| | | 1H), 6.72 (s, 1H), 4.77 (s, 2H), 3.97-3.79 (m, 2H), 3.70 | |
| | | (dd, J = 11.5, 6.8 Hz, 1H). | |
| 192 | 11 | (400 MHz, Methanol-d4) δ 7.32 (dd, J = 5.1, 1.2 Hz, 1H), | 453.8 |
| | | 7.10 (dq, J = 3.3, 1.0 Hz, 1H), 6.98 (dd, J = 5.1, 3.5 Hz, | |
| | | 1H), 6.70 (s, 1H), 4.76 (d, J = 1.0 Hz, 2H), 3.84-3.69 (m, | |
| | | 2H), 3.69-3.60 (m, 1H). | |
| 193 | 11 | (400 MHz, DMSO-d6) δ 8.21 (d, J = 7.3 Hz, 2H), 7.42 (d, | 467.9 |
| | | J = 5.1 Hz, 1H), 7.12 (d, J = 3.5 Hz, 1H), 6.99 (t, J = 4.3 Hz, | |
| | | 1H), 6.75 (s, 1H), 4.74 (d, J = 6.1 Hz, 2H), 3.67 (s, 1H), | |
| | | 3.55 (dd, J = 10.1, 5.0 Hz, 2H), 3.19 (s, 3H). | |
| 194 | 8, 9 | 400 MHz, Methanol-d4) δ 7.29 (dt, J = 5.3, 1.5 Hz, 1H), | 348.0 |
| | | 7.06 (d, J = 3.4 Hz, 1H), 6.97 (ddd, J = 5.1, 3.4, 1.6 Hz, | |
| | | 1H), 6.71 (ddd, J = 17.6, 11.4, 2.1 Hz, 1H), 6.52 (d, J = 1.8 | |
| | | Hz, 1H), 6.34 (dd, J = 17.6, 2.1 Hz, 1H), 5.42 (dd, J = 11.4, | |
| | | 2.1 Hz, 1H), 4.74 (d, J = 2.5 Hz, 2H), 3.44 (d, J = 7.0 Hz, | |
| | | 1H), 3.07-2.91 (m, 2H), 1.18 (dd, J = 6.4, 1.9 Hz, 3H). | |
| 196 | 8, 9 | (400 MHz, Methanol-d4) δ 7.32 (dt, J = 5.2, 1.4 Hz, 1H), | 412.1 |
| | | 7.09 (dt, J = 3.4, 1.1 Hz, 1H), 6.98 (ddd, J = 4.9, 3.5, 1.3 | |
| | | Hz, 1H), 6.73-6.34 (m, 2H), 4.77 (d, J = 2.7 Hz, 2H), 4.20 | |
| | | (q, J = 5.7 Hz, 1H), 4.07-3.99 (m, 3H), 3.48-3.35 (m, 2H). | |
| 197 | 12, 9 | 400 MHz, Methanol-d4) δ 7.34-7.24 (m, 1H), 7.11- | 440.0 |
| | | 7.01 (m, 1H), 7.01-6.91 (m, 1H), 6.56-6.13 (m, 2H), | |
| | | 4.74 (s, 2H), 3.99 (t, J = 6.3 Hz, 2H), 3.46-3.36 (m, 1H), | |
| | | 3.06-2.96 (m, 2H), 2.10 (s, 3H), 1.95-1.61 (m, 2H). | |
| 213 | 8 | (400 MHz, DMSO-d6) δ 7.93 (t, J = 6.3 Hz, 1H), 7.40 (dd, | 402.8 |
| | | J = 5.1, 1.2 Hz, 1H), 7.10 (d, J = 3.4 Hz, 1H), 6.98 (dd, J = | |
| | | 5.1, 3.5 Hz, 1H), 6.60 (s, 1H), 4.92 (s, 1H), 4.72 (d, J = 6.2 | |
| | | Hz, 2H), 4.10 (q, J = 6.4 Hz, 1H), 2.98-2.80 (m, 2H), 1.14 | |
| | | (d, J = 6.1 Hz, 3H). | |
| 214 | 8 | (400 MHz, Methanol-d4) δ 7.30 (dd, J = 5.1, 1.2 Hz, 1H), | 457.9 |
| | | 7.07 (dd, J = 3.5, 1.2 Hz, 1H), 6.97 (dd, J = 5.1, 3.5 Hz, | |
| | | 1H), 6.59 (d, J = 1.5 Hz, 1H), 4.75 (s, 2H), 4.03-3.79 (m, | |
| | | 2H), 3.71 (td, J = 8.5, 7.0 Hz, 1H), 3.50 (dd, J = 8.5, 7.5 Hz, | |
| | | 1H), 3.17 (td, J = 7.7, 5.0 Hz, 1H), 3.06-2.85 (m, 2H), | |
| | | 2.25 (h, J = 7.9 Hz, 1H), 2.10 (dddd, J = 12.2, 8.2, 6.9, 3.8 | |
| | | Hz, 1H), 1.78 (dq, J = 12.4, 8.4 Hz, 1H), 1.17 (d, J = 7.8 Hz, 1H). | |
| 215 | 8 | (400 MHz, Methanol-d4) δ 7.30 (dd, J = 5.1, 1.2 Hz, 1H), | 457.9 |
| | | 7.07 (dq, J = 3.3, 1.1 Hz, 1H), 6.97 (dd, J = 5.1, 3.5 Hz, | |
| | | 1H), 6.60 (s, 1H), 4.76 (d, J = 1.0 Hz, 2H), 4.01-3.83 (m, | |
| | | 2H), 3.72 (td, J = 8.5, 7.1 Hz, 1H), 3.64-3.49 (m, 1H), | |
| | | 3.21-3.06 (m, 2H), 2.95 (dd, J = 14.8, 7.7 Hz, 1H), 2.25 | |
| | | (h, J = 8.0 Hz, 1H), 2.15-2.00 (m, 1H), 1.65 (dq, J = 12.2, | |
| | | 8.4 Hz, 1H). | |
| 216 | 12 | (400 MHz, Methanol-d4) δ 7.77 (d, J = 3.3 Hz, 1H), 7.53 | 482.8 |
| | | (d, J = 3.3 Hz, 1H), 6.58 (s, 1H), 6.54-6.14 (m, 1H), 4.91 | |
| | | (s, 2H), .05-3.90 (m, 2H), 3.45-3.350 (m, 1H), 3.14- | |
| | | 2.91 (m, 2H), 1.94-1.63 (m, 2H). | |
| 219 | | (400 MHz, DMSO-d6) δ 7.93 (dt, J = 15.5, 5.9 Hz, 2H), | 458.0 |
| | | 7.40 (dd, J = 5.0, 1.3 Hz, 1H), 7.13-7.07 (m, 1H), 6.98 | |
| | | (dd, J = 5.1, 3.4 Hz, 1H), 6.61 (s, 1H), 4.71 (d, J = 6.1 Hz, | |
| | | 2H), 3.22-3.08 (m, 3H), 3.00 (dt, J = 12.9, 6.3 Hz, 1H), | |
| | | 2.90 (dd, J = 14.9, 5.0 Hz, 1H), 2.75 (dd, J = 14.8, 8.2 Hz, | |
| | | 1H), 1.82 (s, 3H). | |
| 221 | 8 | (300 MHz, DMSO-d6) δ 8.35 (s, 1H), 8.02 (s, 1H), 7.41 | 403.8 |
| | | (dd, J = 5.1, 1.3 Hz, 1H), 7.11 (dd, J = 3.4, 1.3 Hz, 1H), | |
| | | 6.98 (dd, J = 5.1, 3.4 Hz, 1H), 6.63 (s, 1H), 3.46 (q, J = 6.5 | |
| | | Hz, 1H), 3.11-2.83 (m, 2H), 1.13 (d, J = 6.4 Hz, 3H). | |
| 222 | 12 | 400 MHz, Methanol-d4) δ 7.3.05-2.955-7.250 (m, | 483.9 |
| | | 1H), 7.12-7.02 (m, 1H), 7.02-6.92 (m, 1H), 6.59 (s, | |
| | | 1H), 6.34 (t, J = 75.6 Hz, 1H), 4.05-3.93 (m, 2H), 3.46- | |
| | | 3.36 (m, 1H), 3.00 (m, 2H), 1.93-1.63 (m, 2H). | |
| 223 | 11 | (400 MHz, DMSO-d6) δ 8.39 (t, J = 6.2 Hz, 1H), 7.78 (d, | 438.8 |
| | | J = 3.3 Hz, 1H), 7.66 (d, J = 3.3 Hz, 1H), 6.77 (s, 1H), 4.89 | |
| | | (d, J = 6.4 Hz, 2H), 3.57 (s, 1H), 1.87 (s, 2H), 1.13 (d, J = | |
| | | 6.7 Hz, 3H). | |
| 224 | 11 | (400 MHz, DMSO-d6) δ 8.39 (t, J = 6.3 Hz, 1H), 7.79 (d, | 455.0 |
| | | J = 3.2 Hz, 1H), 7.67 (d, J = 3.3 Hz, 1H), 6.76 (s, 1H), 4.89 | |
| | | (d, J = 6.3 Hz, 2H), 3.65 (dd, J = 10.6, 4.1 Hz, 1H), 3.51 | |
| | | (ddt, J = 29.4, 10.9, 5.7 Hz, 2H). | |
| 226 | 15 | H NMR (400 MHz, Methanol-d4) δ 7.31 (d, J = 5.1 Hz, | 415.8 |
| | | 1H), 7.09 (d, J = 3.5 Hz, 1H), 6.98 (dd, J = 5.0, 3.6 Hz, 1H), | |
| | | 6.66 (s, 1H), 4.79 (s, 2H), 3.21 (d, J = 4.6 Hz, 2H), 1.44 (s, 6H). | |
| 227 | 15 | (400 MHz, Methanol-d4) δ 8.39 (s, 1H), 7.32 (dd, J = 5.1, | 411.8 |
| | | 1.2 Hz, 1H), 7.12-7.06 (m, 1H), 6.98 (dd, J = 5.1, 3.4 Hz, | |
| | | 1H), 6.65 (s, 1H), 3.26 (s, 2H), 1.04 (t, J = 6.6 Hz, 2H), 1.00- | |
| | | 0.93 (m, 2H). | |
| 229 | 8 | (300 MHz, Methanol-d4) δ 7.30 (dd, J = 5.1, 1.3 Hz, 1H), | 402.8 |
| | | 7.07 (dd, J = 3.5, 1.2 Hz, 1H), 6.97 (dd, J = 5.1, 3.5 Hz, | |
| | | 1H), 6.60 (s, 1H), 4.76 (s, 2H), 2.97 (s, 2H), 1.17 (s, 2H). | |
| 230 | 8 | (400 MHz, Methanol-d4) δ 7.35-7.25 (m, 1H), 7.09- | 404.9 |
| | | 7.06 (m, 1H), 7.00-6.94 (m, 1H), 6.60-6.57 (m, 1H), | |
| | | 4.76 (s, 2H), 3.44-3.36 (m, 1H), 3.02-2.89 (m, 2H). | |
| 231 | 8 | 400 MHz, Methanol-d4) δ 7.35-7.25 (m, 1H), 7.09- | 404.8 |
| | | 7.06 (m, 1H), 7.00-6.94 (m, 1H), 6.60-6.57 (m, 1H), | |
| | | 4.76 (s, 2H), 3.44-3.36 (m, 1H), 3.02-2.89 (m, 2H). | |
| 232 | 8 | (400 MHz, Methanol-d4) δ 7.35-7.25 (m, 1H), 7.09- | 403.0 |
| | | 7.06 (m, 1H), 7.00-6.94 (m, 1H), 6.60-6.57 (m, 1H), | |
| | | 4.76 (s, 2H), 3.44-3.36 (m, 1H), 3.02-2.89 (m, 2H). | |
| 233 | 8, 9 | (400 MHz, Methanol-d4) δ 7.30 (dd, J = 5.1, 1.2 Hz, 1H), | 379.0 |
| | | 7.07 (dd, J = 3.5, 1.2 Hz, 1H), 6.97 (dd, J = 5.1, 3.5 Hz, | |
| | | 1H), 6.56 (s, 1H), 4.75 (s, 2H), 3.48-3.34 (m, 2H), 3.28 | |
| | | (s, 1H), 3.12 (dd, J = 14.8, 5.8 Hz, 1H), 2.98 (dd, J = 14.8, | |
| | | 7.2 Hz, 1H). | |
| 234 | 8 | (300 MHz, Methanol-d4) δ 7.30 (dd, J = 5.1, 1.2 Hz, 1H), | 404.8 |
| | | 7.08 (dd, J = 3.5, 1.2 Hz, 1H), 7.00-6.96 (m, 1H), 6.60 (s, | |
| | | 1H), 2.97 (d, J = 3.0 Hz, 2H), 1.17 (s, 3H). | |
| 235 | 8 | (300 MHz, Methanol-d4) δ 7.51-7.42 (m, 1H), 6.66 (s, | 410.8 |
| | | 1H), , 6.44-6.29 (m, 5H), 4.56 (s, 2H), 3.89 (t, J = 7.2 Hz, | |
| | | 2H), 3.11 (dd, J = 8.5, 6.7 Hz, 2H), 2.21 (tdd, J = 7.8, 6.4, | |
| | | 1.2 Hz, 2H). | |
| 237 | 8, 16 | (300 MHz, Methanol-d4) δ 7.33 (dd, J = 5.1, 1.3 Hz, 1H), | 378.9 |
| | | 7.11 (dq, J = 3.3, 1.0 Hz, 1H), 7.00 (dd, J = 5.1, 3.5 Hz, | |
| | | 1H), 6.64 (s, 1H), 4.79 (d, J = 1.0 Hz, 2H), 4.70 (q, J = 6.1 | |
| | | Hz, 0H), 3.98 (dd, J = 15.3, 5.3 Hz, 1H), 1.45 (dd, J = 24.2, | |
| | | 6.3 Hz, 3H). | |
| 238 | 8, 16 | (400 MHz, Methanol-d4) δ 7.45 (dd, J = 1.8, 0.9 Hz, 1H), | 333.1 |
| | | 6.72 (s, 1H), 6.39-6.31 (m, 2H), 4.56 (s, 2H), 4.14 (q, J = | |
| | | 6.6 Hz, 1H), 1.46 (d, J = 6.6 Hz, 3H). | |
| 239 | 11 | (400 MHz, Methanol-d4) δ 7.45 (dd, J = 1.8, 0.9 Hz, 1H), | 376.0 |
| | | 6.77 (s, 1H), 6.36 (qd, J = 3.2, 1.3 Hz, 2H), 4.56 (s, 2H), | |
| | | 3.85 (ddt, J = 17.6, 13.7, 6.8 Hz, 1H), 1.28 (d, J = 6.8 Hz, 3H). | |
| 240 | 8, 9 | (300 MHz, Methanol-d4) δ 7.30 (dd, J = 5.1, 1.2 Hz, 1H), | 394.0 |
| | | 7.07 (dq, J = 3.3, 1.1 Hz, 1H), 6.97 (dd, J = 5.1, 3.5 Hz, | |
| | | 1H), 6.52 (s, 1H), 5.08 (qd, J = 6.5, 2.7 Hz, 0H), 4.73 (d, J = | |
| | | 1.0 Hz, 2H), 3.91 (dddd, J = 21.3, 8.3, 5.3, 2.7 Hz, 1H), | |
| | | 3.36 (dd, J = 15.8, 5.3 Hz, 1H), 3.18 (dd, J = 15.8, 8.8 Hz, | |
| | | 1H), 1.76 (tt, J = 8.3, 5.3 Hz, 1H), 1.46 (dd, J = 24.2, 6.5 | |
| | | Hz, 3H), 1.12-0.88 (m, 4H). | |
| 242 | 8 | (400 MHz, Methanol-d4) δ 7.33-7.28 (m, 1H), 7.12- | 436.0 |
| | | 7.02 (m, 1H), 7.02-6.92 (m, 1H), 6.62-6.55 (m, 1H), | |
| | | 5.88-5.78 (m, 1H), 4.74 (s, 2H), 3.55-3.41 (m, 1H), | |
| | | 3.22-3.12 (m, 1H), 3.00 (m, 1H). | |
| 243 | 8 | 400 MHz, Methanol-d4) δ 7.49-7.39 (m, 1H), 6.64 (d, | 420.0 |
| | | J = 1.6 Hz, 1H), 6.39-6.30 (m, 2H), 5.88-5.78 (m, 1H), | |
| | | 4.54 (s, 2H), 3.56-3.39 (m, 1H), 3.22-3.12 (m, 1H), | |
| | | 3.04-2.94 (m, 1H). | |
| 244 | 8, 9 | 400 MHz, Methanol-d4) δ 7.35-7.25 (m, 1H), 7.12- | 402.0 |
| | | 7.02 (m, 1H), 7.02-6.92 (m, 1H), 6.56 (s, 1H), 4.74 (d, J = | |
| | | 1.1 Hz, 2H), 3.86 (s, 1H), 3.54-3.46 (m, 1H), 3.46-3.35 | |
| | | (m, 2H), 3.34-3.25 (m, 1H), 3.14-3.05 (m, 1H), 3.02- | |
| | | 2.92 (m, 1H), 0.57-0.37 (m, 4H). | |
| 246 | 8 | 400 MHz, Methanol-d4) 87.32-7.28 (m, 1H), 7.12- | 486.0 |
| | | 7.02 (m, 1H), 7.02-6.92 (m, 1H), 6.59 (d, J = 0.6 Hz, 1H), | |
| | | 4.09-3.93 (m, 2H), 3.58-3.48 (m, 1H), 3.17-3.07 (m, | |
| | | 1H), 3.00 (m, 1H). | |
| 248 | 8, 9 | (400 MHz, DMSO-d6) δ 7.91 (t, J = 6.2 Hz, 1H), 7.40 (d, | 362.1 |
| | | J = 5.0 Hz, 1H), 7.10 (d, J = 3.5 Hz, 1H), 6.98 (dd, J = 5.1, | |
| | | 3.5 Hz, 1H), 6.57 (s, 1H), 4.71 (t, J = 6.2 Hz, 3H), 4.42 (s, | |
| | | 1H), 3.30 (s, 2H), 3.16 (dq, J = 11.1, 5.6 Hz, 1H), 3.00 (dd, | |
| | | J = 14.7, 5.1 Hz, 1H), 2.75 (dd, J = 14.7, 8.3 Hz, 1H), 1.96 | |
| | | (s, 2H). | |
| 249 | 8 | (300 MHz, Methanol-d4) δ 7.33 (dd, J = 5.1, 1.2 Hz, 1H), | 436.9 |
| | | 7.10 (dd, J = 3.5, 1.2 Hz, 1H), 7.00 (dd, J = 5.1, 3.5 Hz, | |
| | | 1H), 6.63 (d, J = 1.2 Hz, 1H), 3.57 (td, J = 6.6, 4.4 Hz, 1H), | |
| | | 3.48 (dd, J = 9.6, 4.2 Hz, 1H), 3.41-3.34 (m, 1H), 3.17 | |
| | | (dd, J = 15.0, 6.5 Hz, 1H), 3.05 (dd, J = 15.0, 7.1 Hz, 1H). | |
| 250 | 8, 9 | (400 MHz, Methanol-d4) δ 7.31 (dd, J = 5.1, 1.2 Hz, 1H), | 380.9 |
| | | 7.09 (dd, J = 3.5, 1.2 Hz, 1H), 6.98 (dd, J = 5.1, 3.5 Hz, | |
| | | 1H), 6.61 (s, 1H), 3.99 (s, 1H), 3.88 (tdd, J = 7.2, 5.8, 3.5 | |
| | | Hz, 1H), 3.64 (dd, J = 10.4, 3.5 Hz, 1H), 3.48 (dd, J = 10.4, | |
| | | 5.8 Hz, 1H), 3.38 (dd, J = 15.3, 7.4 Hz, 1H), 3.32 (s, 1H). | |
| 252 | 8, 9 | (400 MHz, Methanol-d4) δ 7.30 (dd, J = 5.1, 1.2 Hz, 1H), | 362.0 |
| | | 7.08 (dd, J = 3.5, 1.1 Hz, 1H), 6.98 (dd, J = 5.1, 3.5 Hz, | |
| | | 1H), 6.56 (s, 1H), 5.35 (p, J = 1.7 Hz, 1H), 5.16 (dd, J = 2.0, | |
| | | 1.1 Hz, 1H), 4.76 (s, 2H), 3.82-3.69 (m, 1H), 3.19 (qd, J = | |
| | | 15.4, 7.1 Hz, 2H), 2.18 (d, J = 1.3 Hz, 3H), 1.33 (d, J = 6.5 | |
| | | Hz, 3H). | |
| 253 | 8, 9 | (400 MHz, Methanol-d4) δ 7.28 (dd, J = 5.1, 1.3 Hz, 1H), | 362.0 |
| | | 7.06 (dd, J = 3.5, 1.2 Hz, 1H), 6.96 (dd, J = 5.1, 3.5 Hz, | |
| | | 1H), 6.85 (dq, J = 15.7, 6.7 Hz, 1H), 6.51 (s, 1H), 6.37 (dq, | |
| | | J = 15.8, 1.7 Hz, 1H), 4.73 (s, 2H), 3.42 (h, J = 6.5 Hz, 1H), | |
| | | 3.04-2.86 (m, 2H), 1.91 (dd, J = 6.6, 1.8 Hz, 3H), 1.18 (d, | |
| | | J = 6.4 Hz, 3H). | |
| 254 | 8, 9 | (400 MHz, Chloroform-d) δ 7.16 (dd, J = 5.1, 1.2 Hz, 1H), | 361.9 |
| | | 7.02-6.96 (m, 1H), 6.91 (dd, J = 5.1, 3.5 Hz, 1H), 6.43 (s, | |
| | | 1H), 6.34 (s, 1H), 6.26-6.17 (m, 1H), 5.97 (dq, J = 11.1, | |
| | | 6.8 Hz, 1H), 4.58 (d, J = 5.3 Hz, 2H), 3.67 (q, J = 6.4 Hz, | |
| | | 1H), 2.98 (qd, J = 15.7, 5.8 Hz, 2H), 1.62 (dd, J = 6.9, 1.7 | |
| | | Hz, 3H), 1.28 (d, J = 6.6 Hz, 3H). | |
| 256 | 8, 9 | (400 MHz, Methanol-d4) δ 7.61 (dd, J = 3.5, 1.2 Hz, 1H), | 404.0 |
| | | 7.49 (dd, J = 5.2, 1.2 Hz, 1H), 7.30 (dd, J = 5.1, 1.2 Hz, | |
| | | 1H), 7.17 (dd, J = 5.2, 3.6 Hz, 1H), 7.08 (dd, J = 3.6, 1.2 | |
| | | Hz, 1H), 6.98 (dd, J = 5.1, 3.5 Hz, 1H), 6.57 (s, 1H), 3.54 | |
| | | (h, J = 6.5 Hz, 1H), 3.21-3.10 (m, 2H), 1.20 (d, J = 6.5 Hz, 3H). | |
| 257 | 8, 9 | (400 MHz, Methanol-d4) δ 7.92 (d, J = 3.3 Hz, 1H), 7.66 | 405.0 |
| | | (d, J = 3.3 Hz, 1H), 7.30 (dd, J = 5.1, 1.2 Hz, 1H), 7.09 (dq, | |
| | | J = 3.3, 1.0 Hz, 1H), 6.98 (dd, J = 5.1, 3.5 Hz, 1H), 6.61 (s, | |
| | | 1H), 4.78 (d, J = 1.0 Hz, 2H), 3.67 (h, J = 6.5 Hz, 1H), 3.58- | |
| | | 3.45 (m, 2H), 1.28 (d, J = 6.5 Hz, 3H). | |
| 260 | 8, 9 | (400 MHz, Methanol-d4) δ 7.76 (dt, J = 2.8, 1.2 Hz, 1H), | 404.0 |
| | | 7.53 (ddd, J = 5.0, 3.0, 1.1 Hz, 1H), 7.47 (dt, J = 5.0, 1.3 | |
| | | Hz, 1H), 7.30 (dt, J = 5.1, 1.0 Hz, 1H), 7.08 (dt, J = 3.4, 1.0 | |
| | | Hz, 1H), 7.02-6.92 (m, 1H), 6.55 (d, J = 1.2 Hz, 1H), 4.77 | |
| | | (d, J = 1.2 Hz, 2H), 3.48 (h, J = 6.6 Hz, 1H), 3.17-2.99 (m, | |
| | | 2H), 1.16 (d, J = 6.4 Hz, 3H). | |
| 262 | 8, 9 | (400 MHz, Methanol-d4) δ 8.03 (d, J = 0.9 Hz, 1H), 7.34- | 389.0 |
| | | 7.27 (m, 2H), 7.09 (dq, J = 3.3, 1.1 Hz, 1H), 6.98 (dd, J = | |
| | | 5.1, 3.4 Hz, 1H), 6.62 (s, 1H), 4.77 (d, J = 1.0 Hz, 2H), 3.55 | |
| | | (h, J = 6.5 Hz, 1H), 3.34 (d, J = 6.1 Hz, 2H), 1.22 (d, J = 6.5 | |
| | | Hz, 3H). | |
| 263 | 8, 9 | (400 MHz, Chloroform-d) δ 8.85 (d, J = 2.1 Hz, 1H), 8.55 | 404.9 |
| | | (d, J = 2.1 Hz, 1H), 7.29 (dd, J = 5.1, 1.2 Hz, 1H), 7.09 (dd, | |
| | | J = 3.5, 1.1 Hz, 1H), 7.02 (dd, J = 5.1, 3.5 Hz, 1H), 6.54 (s, | |
| | | 1H), 5.08 (t, J = 5.5 Hz, 1H), 4.70 (d, J = 5.6 Hz, 2H), 3.44 | |
| | | (ddt, J = 15.7, 13.3, 6.6 Hz, 3H), 1.21 (d, J = 6.0 Hz, 3H). | |
| 264 | 6 | (400 MHz, Methanol-d4) δ 7.35-7.25 (m, 1H), 7.12- | 389.0 |
| | | 7.02 (m, 1H), 7.00-6.93 (m, 1H), 6.58 (d, J = 2.0 Hz, 1H), | |
| | | 4.75 (s, 2H), 3.45-3.34 (m, 2H), 3.30-3.27 (m, 1H), | |
| | | 3.10-3.00 (m, 1H), 2.98-2.88 (m, 1H). | |
| 265 | 6 | (400 MHz, Methanol-d4) δ 7.5-7.4 (m, 1H), 6.64 (s, 1H), | 373.0 |
| | | 6.39-6.29 (m, 2H), 4.55 (s, 2H), 3.49-3.37 (m, 2H), | |
| | | 3.35-3.32 (m, 1H), 3.13-3.03 (m, 1H), 3.01-2.90 (m, 1H). | |
| 266 | 8, 9 | (400 MHz, Methanol-d4) δ 7.35-7.25 (m, 1H), 7.12- | 390.0 |
| | | 7.02 (m, 1H), 7.02-6.92 (m, 1H), 6.56 (d, J = 2.2 Hz, 1H), | |
| | | 4.75 (t, J = 1.2 Hz, 2H), 3.88 (s, 1H), 3.60-3.40 (m, 3H), | |
| | | 3.32-3.31 (m, 1H), 3.31-3.29 (m, 2H), 3.15-2.96 (m, | |
| | | 2H), 1.86-1.60 (m, 2H). | |
| 267 | 8 | (400 MHz, Methanol-d4) δ 7.35-7.25 (m, 1H), 7.10- | 448.5 |
| | | 7.04 (m, 1H), 7.00-6.94 (m, 1H), 6.63-6.53 (m, 1H), | |
| | | 4.75 (s, 2H), 3.59-3.45-3.354 (m, 2H), 3.40 (m, 1H), | |
| | | 3.06-2.88 (m, 2H), 1.82-1.57 (m, 2H). | |
| 268 | 8, 9 | (300 MHz, Methanol-d4) δ 7.30 (dd, J = 5.1, 1.2 Hz, 1H), | 347.0 |
| | | 7.07 (dt, J = 3.5, 1.1 Hz, 1H), 6.97 (dd, J = 5.1, 3.5 Hz, 1H), | |
| | | 6.57 (s, 1H), 4.75 (d, J = 1.0 Hz, 2H), 3.86 (s, 1H), 3.02 (d, | |
| | | J = 1.7 Hz, 2H), 1.18 (s, 2H). | |
| 269 | 8.9 | (400 MHz, Methanol-d4) δ 7.35-7.25 (m, 1H), 7.12- | 349.0 |
| | | 7.02 (m, 1H), 7.02-6.92 (m, 1H), 6.56 (s, 1H), 4.75 (s, | |
| | | 2H), 3.85 (s, 1H), 3.43-3.37 (m, 1H), 3.04-2.94 (m, 2H). | |
| 270 | 8, 9 | (400 MHz, Methanol-d4) δ 7.35-7.25 (m, 1H), 7.12- | 349.0 |
| | | 7.02 (m, 1H), 7.02-6.92 (m, 1H), 6.56 (s, 1H), 4.75 (s, | |
| | | 2H), 3.85 (s, 1H), 3.43-3.37 (m, 1H), 3.04-2.94 (m, 2H). | |
| 271 | 8, 9 | (400 MHz, Methanol-d4) δ 7.35-7.25 (m, 1H), 7.12- | 349.0 |
| | | 7.02 (m, 1H), 7.02-6.92 (m, 1H), 6.56 (s, 1H), 4.75 (s, | |
| | | 2H), 3.85 (s, 1H), 3.43-3.37 (m, 1H), 3.04-2.94 (m, 2H). | |
| 273 | 6 | (400 MHz, Methanol-d4) δ 7.30 (dd, J = 5.1, 1.3 Hz, 1H), | 403.0 |
| | | 7.08 (d, J = 3.5 Hz, 1H), 6.97 (dd, J = 5.1, 3.5 Hz, 1H), 6.59 | |
| | | (s, 1H), 4.75(S, 2H), 3.60-3.45 (m, 2H), 3.40 (p, J = 6.5 | |
| | | Hz, 1H), 3.02 (dd, J = 14.9, 6.0 Hz, 1H), 2.94 (dd, J = 14.9, | |
| | | 7.1 Hz, 1H), 1.76 (ddt, J = 12.0, 6.6, 5.1 Hz, 1H), 1.72- | |
| | | 1.59 (m, 1H). | |
| 274 | 6 | (400 MHz, Methanol-d4) δ 7.30 (dd, J = 5.1, 1.2 Hz, 1H), | 400.0 |
| | | 7.07 (dt, J = 3.4, 1.1 Hz, 1H), 6.97 (dd, J = 5.1, 3.5 Hz, 1H), | |
| | | 6.59 (s, 1H), 4.76 (d, J = 1.0 Hz, 2H), 3.60-3.45 (m, 2H), | |
| | | 3.45-3.35 (m, 1H), 3.02 (dd, J = 14.9, 6.0 Hz, 1H), 2.94 | |
| | | (dd, J = 14.9, 7.2 Hz, 1H), 1.76 (ddt, J = 14.3, 6.9, 5.2 Hz, | |
| | | 1H), 1.66 (dddd, J = 14.4, 8.1, 6.7, 5.3 Hz, 1H). | |
| 275 | 2, 3 | (400 MHz, Methanol-d4) δ 7.27 (dd, J = 5.1, 1.3 Hz, 1H), | 418.0 |
| | | 7.02 (d, J = 1.3 Hz, 1H), 6.94 (dd, J = 5.1, 3.5 Hz, 1H), 5.04 | |
| | | (s, 2H), 4.59 (s, 1H), 3.49-3.43 (m, 0H), 3.07-2.92 (m, | |
| | | 2H), 2.03 (s, 1H), 1.14 (d, J = 6.5 Hz, 3H). | |
| 276 | 2, 3, 9 | (400 MHz, Methanol-d4) δ 7.26 (dd, J = 5.1, 1.2 Hz, 1H), | 364.0 |
| | | 7.01 (dq, J = 3.3, 1.0 Hz, 1H), 6.94 (dd, J = 5.1, 3.5 Hz, | |
| | | 1H), 5.03 (d, J = 1.4 Hz, 2H), 3.87 (s, 1H), 3.37 (dt, J = | |
| | | 13.2, 6.5 Hz, 1H), 3.07-2.87 (m, 2H), 1.10 (d, J = 6.4 Hz, 3H). | |
| 277 | 2, 3 | (400 MHz, Methanol-d4) δ 7.27 (dd, J = 5.1, 1.3 Hz, 1H), | 451.8 |
| | | 7.02 (dt, J = 3.4, 1.1 Hz, 1H), 6.94 (dd, J = 5.1, 3.5 Hz, 1H), | |
| | | 5.04 (s, 2H), 4.53 (ddt, J = 47.3, 11.3, 6.3 Hz, 1H), 3.29- | |
| | | 3.21 (m, 1H), 3.11 (dd, J = 15.0, 5.0 Hz, 1H), 2.90 (dd, J = | |
| | | 15.1, 8.5 Hz, 1H), 1.35 (dd, J = 24.3, 6.3 Hz, 3H). | |
| 278 | 2, 3, 9 | 1 H NMR (400 MHz, Methanol-d4) δ 7.26 (dd, J = 5.1, 1.2 | 396.0 |
| | | Hz, 1H), 7.02 (dt, J = 3.4, 1.1 Hz, 1H), 6.94 (dd, J = 5.1, 3.5 | |
| | | Hz, 1H), 4.53 (dqd, J = 47.4, 6.4, 5.0 Hz, 1H), 3.89 (s, 1H), | |
| | | 3.29-3.24 (m, 1H), 3.16 (dd, J = 15.0, 5.1 Hz, 1H), 3.02- | |
| | | 2.92 (m, 1H), 1.35 (dd, J = 24.3, 6.3 Hz, 3H). | |
| 279 | 2 | (300 MHz, DMSO-d6): δ 8.48 (s, 1H), 8.11-8.07 (t, J = 6.3 | 343.0 |
| | | Hz, 1H), 7.42-7.40 (m, 1H), 7.12-7.10 (m, 1H), 7.00-6.97 | |
| | | (m, 1H), 6.67 (s, 1H), 4.75-4.73 (d, J = 6.2 Hz, 2H). | |
| 280 | 2 | (300 MHz, DMSO-d6): δ 8.53-8.50 (m, 3H), 8.14-8.10 (t, | 338.0 |
| | | J = 6.4 Hz, 1H), 7.36-7.34 (m, 2H), 6.52 (s, 1H), 4.62-4.59 | |
| | | (d, J = 6.4 Hz, 2H). | |
| 281 | | (400 MHz, Methanol-d4) δ 7.62 (s, 1H), 7.31 (dd, J = 5.1, | 402.0 |
| | | 1.2 Hz, 1H), 7.11 (dt, J = 3.5, 1.1 Hz, 1H), 6.99 (dd, J = 5.1, | |
| | | 3.5 Hz, 1H), 6.75 (s, 1H), 4.80 (s, 2H), 3.43 (s, 1H), 3.37 (s, | |
| | | 1H), 3.02 (s, 2H), 1.66 (d, J = 8.2 Hz, 1H), 1.40 (dd, J = | |
| | | 14.7, 7.3 Hz, 1H), 1.12 (d, J = 6.9 Hz, 1H), 1.07 (d, J = 7.1 | |
| | | Hz, 1H), 0.93 (t, J = 7.3 Hz, 1H), 0.84-0.81 (m, 1H). | |
| 282 | 6 | (400 MHz, Methanol-d4) δ 7.37-7.27 (m, 1H), 7.14- | 390.0 |
| | | 7.04 (m, 1H), 7.04-6.94 (m, 1H), 6.60 (s, 1H), 4.75 (s, | |
| | | 2H), 3.40 (d, J = 9.5 Hz, 1H), 3.31-3.29 (m, 1H), 3.07 (d, | |
| | | J = 15.1 Hz, 1H), 2.95 (d, J = 15.0 Hz, 1H). | |
| 283 | 6 | -(400 MHz, Methanol-d4) δ 7.35-7.25 (m, 1H), 7.12- | 387.0 |
| | | 7.02 (m, 1H), 7.02-6.92 (m, 1H), 6.58 (s, 1H), 4.75 (s, | |
| | | 2H), 3.41-3.32 (m, 5H), 3.05 (d, J = 15.0 Hz, 1H), 2.92 | |
| | | (d, J = 15.0 Hz, 1H). | |
| 284 | 8 | (400 MHz, Methanol-d4) δ 7.37-7.27 (m, 1H), 7.14- | 434.0 |
| | | 7.04 (m, 1H), 7.04-6.94 (m, 1H), 6.60 (s, 1H), 4.77 (s, | |
| | | 2H), 3.39 (d, J = 9.4 Hz, 1H), 3.31 (d, J = 9.2 Hz, 1H), 3.07 | |
| | | (d, J = 14.9 Hz, 1H), 2.94 (d, J = 14.9 Hz, 1H). | |
| 285 | 8 | 400 MHz, Methanol-d4) 7.35-7.25 (m, 1H), 7.12-7.02 | 433.0 |
| | | (m, 1H), 7.02-6.92 (m, 1H), 6.58 (t, J = 3.9 Hz, 1H), 4.75 | |
| | | (s, 2H), 3.41-3.33 (m, 4H), 3.29-3.26 (m, 1H), 3.05 (d, | |
| | | J = 14.7 Hz, 1H), 2.92 (d, J = 15.0 Hz, 1H). | |
| 286 | 8, 9 | 400 MHz, Methanol-d4) δ 7.35-7.250 (m, 1H), 7.12- | 380.0 |
| | | 7.02 (m, 1H), 7.02-6.92 (m, 1H), 6.56 (s, 1H), 4.75 (s, | |
| | | 2H), 3.86 (s, 1H), 3.38 (d, J = 9.4 Hz, 1H), 3.29 (s, 1H), | |
| | | 3.12 (d, J = 14.8 Hz, 1H), 2.98 (d, J = 14.9 Hz, 1H). | |
| 287 | 8, 9 | (400 MHz, Methanol-d4) δ 7.35-7.25 (m, 1H), 7.12- | 377.0 |
| | | 7.02 (m, 1H), 7.02-6.92 (m, 1H), 6.56 (s, 1H), 4.75 (s, | |
| | | 2H), 3.87 (s, 1H), 3.40 (d, J = 9.5 Hz, 1H), 3.36 (s, 3H), | |
| | | 3.32 (s, 1H), 3.13 (d, J = 14.9 Hz, 1H), 3.00 (d, J = 14.8 Hz, 1H). | |
| 290 | | 1 H NMR (400 MHz, Methanol-d4) δ 8.49 (s, 1H), 7.31 | 411.9 |
| | | (dd, J = 5.1, 1.2 Hz, 1H), 7.09 (dd, J = 3.5, 1.2 Hz, 1H), | |
| | | 6.99 (dd, J = 5.1, 3.5 Hz, 1H), 6.63 (s, 1H), 4.79 (s, 2H), | |
| | | 4.09 (q, J = 8.5 Hz, 1H), 3.90 (q, J = 9.1 Hz, 1H), 2.44- | |
| | | 2.20 (m, 3H), 2.15 (q, J = 9.7 Hz, 1H). | |
| 291 | | 1 H NMR (400 MHz, Methanol-d4) δ 8.49 (s, 1H), 7.31 | 411.9 |
| | | (dd, J = 5.1, 1.2 Hz, 1H), 7.09 (dd, J = 3.5, 1.2 Hz, 1H), | |
| | | 6.99 (dd, J = 5.1, 3.5 Hz, 1H), 6.63 (s, 1H), 4.79 (s, 2H), | |
| | | 4.09 (q, J = 8.5 Hz, 1H), 3.90 (q, J = 9.1 Hz, 1H), 2.44- | |
| | | 2.20 (m, 3H), 2.15 (q, J = 9.7 Hz, 1H). | |
| 292 | 8 | ((400 MHz, Methanol-d4) δ 7.35-7.25 (m, 1H), 7.12- | 402 |
| | | 7.02 (m, 1H), 7.02-6.92 (m, 1H), 6.58 (s, 1H), 4.76 (s, | |
| | | 2H), 3.43-3.33 (m, 1H), 1.15 (d, J = 6.5 Hz, 3H). | |
| 293 | 6 | (300 MHz, Methanol-d4) δ 7.30 (d, J = 5.1 Hz, 1H), 7.07 | 358.0 |
| | | (d, J = 3.6 Hz, 1H), 6.97 (t, J = 4.4 Hz, 1H), 6.58 (s, 1H), | |
| | | 4.76 (s, 2H), 3.39 (s, 1H), 2.93 (d, J = 7.5 Hz, 1H), 1.16 (d, | |
| | | J = 6.4 Hz, 3H). | |
| 295 | | (400 MHz, Methanol-d4) δ 7.30 (dd, J = 5.1, 1.2 Hz, 1H), | 439.0 |
| | | 7.07 (dq, J = 3.3, 1.0 Hz, 1H), 6.97 (dd, J = 5.1, 3.5 Hz, | |
| | | 1H), 6.58 (s, 1H), 3.76 (dd, J = 8.9, 2.2 Hz, 1H), 3.44 (dq, | |
| | | J = 8.7, 7.0 Hz, 1H), 2.41 (s, 3H), 1.49 (d, J = 7.0 Hz, 3H). | |
| 296 | 8 | (300 MHz, Methanol-d4) δ 7.30 (dd, J = 5.1, 1.2 Hz, 2H), | 432.0 |
| | | 7.07 (dd, J = 3.5, 1.2 Hz, 2H), 6.97 (dd, J = 5.1, 3.5 Hz, | |
| | | 2H), 6.58 (s, 2H), 4.78-4.72 (m, 4H), 3.44-3.34 (m, | |
| | | 8H),3.06 (dd, J = 14.9, 6.1 Hz, 2H), 2.93 (dd, J = 14.9, 7.4 | |
| | | Hz, 2H). | |
| 297 | 8 | (300 MHz, Methanol-d4) δ 7.30 (dd, J = 5.1, 1.2 Hz, 2H), | 435.0 |
| | | 7.08 (dd, J = 3.6, 1.2 Hz, 2H), 6.97 (dd, J = 5.1, 3.5 Hz, | |
| | | 2H), 6.59 (s, 2H), 4.76 (s, 3H), 4.59 (s, 1H), 3.40 (t, J = | |
| | | 6.8Hz, 2H), 3.07 (dd, J = 14.9, 6.2 Hz, 2H), 2.94 (dd, J = | |
| | | 14.9, 7.4 Hz, 2H). | |
| 298 | 8 | (400 MHz, Methanol-d4) δ 7.30 (dd, J = 5.1, 1.2 Hz, 1H), | 467.8 |
| | | 7.07 (dd, J = 3.4, 1.2 Hz, 1H), 6.97 (dd, J = 5.1, 3.5 Hz, | |
| | | 1H), 6.58 (s, 1H), 6.42 (s, 1H), 4.75 (s, 2H), 3.47 (dd, J = | |
| | | 7.7, 5.8 Hz, 1H), 3.10 (dd, J = 15.0, 5.8 Hz, 1H), 2.97 (dd, | |
| | | J = 15.0, 7.8 Hz, 1H). | |
| 299 | 8 | (400 MHz, Methanol-d4) δ 7.30 (dd, J = 5.1, 1.3 Hz, 1H), | 419.8 |
| | | 7.08 (dd, J = 3.5, 1.2 Hz, 1H), 6.97 (dd, J = 5.1, 3.5 Hz, | |
| | | 1H), 6.59 (s, 1H), 4.75 (s, 2H), 3.46 (ddd, J = 18.8, 7.5, 6.1 | |
| | | Hz, 1H), 3.10 (dd, J = 15.0, 6.1 Hz, 1H), 2.98 (dd, J = 15.0, | |
| | | 7.6 Hz, 1H). | |
| 300 | 8, 9 | (300 MHz, Methanol-d4) δ 7.32 (dd, J = 5.1, 1.3 Hz, 5H), | 378.0 |
| | | 7.09 (dq, J = 3.4, 1.0 Hz, 5H), 6.99 (dd, J = 5.1, 3.5 Hz, | |
| | | 5H), 6.58 (s, 5H), 4.77 (d, J = 1.0 Hz, 11H), 4.64 (s, | |
| | | 1H),3.88 (s, 4H), 3.49-3.40 (m, 6H), 3.37 (s, 17H), 3.14 | |
| | | (dd, J = 14.8, 6.1 Hz, 6H), 3.00 (dd, J = 14.8, 7.4 Hz, 5H). | |
| 301 | 8, 9 | (400 MHz, Methanol-d4) δ 7.30 (dd, J = 5.1, 1.3 Hz, 2H), | 381.0 |
| | | 7.07 (dd, J = 3.5, 1.2 Hz, 2H), 6.97 (dd, J = 5.1, 3.5 Hz, | |
| | | 2H), 6.56 (s, 2H), 4.77-4.73 (m, 4H), 4.59 (s, 1H), 3.86 | |
| | | (s,1H), 3.42 (dd, J = 7.4, 6.1 Hz, 2H), 3.12 (dd, J = 14.8, | |
| | | 6.1 Hz, 2H), 2.98 (dd, J = 14.8, 7.4 Hz, 2H). | |
| 302 | 8, 9 | (400 MHz, Methanol-d4) δ 7.30 (dd, J = 5.1, 1.3 Hz, 1H), | 413.9 |
| | | 7.07 (dd, J = 3.5, 1.1 Hz, 1H), 6.97 (dd, J = 5.1, 3.5 Hz, | |
| | | 1H), 6.56 (s, 1H), 4.74 (s, 2H), 3.87 (s, 1H), 3.50 (dd, J = | |
| | | 7.7, 5.7 Hz, 1H), 3.16 (dd, J = 15.0, 5.8 Hz, 1H), 3.02 (dd, | |
| | | J = 15.0, 7.8 Hz, 1H). | |
| 304 | 8 | (400 MHz, Methanol-d4) δ 7.30 (dd, J = 5.1, 1.2 Hz, 1H), | 431.8 |
| | | 7.07 (dd, J = 3.5, 1.1 Hz, 1H), 6.97 (dd, J = 5.1, 3.5 Hz, | |
| | | 1H), 6.58 (s, 1H), 4.75 (s, 2H), 3.38 (d, J = 7.5 Hz, 2H), | |
| | | 3.35 (s, 3H), 3.33 (s, 1H). | |
| 305 | 8 | (400 MHz, Methanol-d4) δ 8.48 (s, 1H), 7.31 (dt, J = 5.0, | 421.8 |
| | | 1.2 Hz, 1H), 7.11-7.06 (m, 1H), 7.00-6.95 (m, 1H), 6.63 | |
| | | (dd, J = 4.0, 1.7 Hz, 1H), 4.76 (d, J = 2.1 Hz, 2H), 4.67- | |
| | | 4.39 (m, 2H), 3.73 (dt, J = 20.9, 4.6 Hz, 1H). | |
| 310 | | (400 MHz, Methanol-d4) δ 8.30 (s, 1H), 7.74 (s, 1H), 7.30 | 388.8 |
| | | (dd, J = 5.1, 1.2 Hz, 1H), 7.12-7.05 (m, 1H), 6.98 (dd, J = | |
| | | 5.1, 3.5 Hz, 1H), 6.59 (s, 1H), 4.77 (d, J = 1.0 Hz, 2H), 3.45 | |
| | | (q, J = 6.5 Hz, 1H), 3.20 (d, J = 6.9 Hz, 2H), 1.17 (d, J = 6.4 | |
| | | Hz, 3H). | |
| 311 | 8, 9 | (400 MHz, Methanol-d4) δ 8.54 (s, 1H), 7.62 (d, J = 1.9 | 387.9 |
| | | Hz, 1H), 7.31 (dd, J = 5.0, 1.2 Hz, 1H), 7.20 (d, J = 3.3 Hz, | |
| | | 1H), 7.09 (dd, J = 3.5, 1.2 Hz, 1H), 6.98 (dd, J = 5.1, 3.5 | |
| | | Hz, 1H), 6.63-6.57 (m, 2H), 3.77 (p, J = 6.7 Hz, 1H), 3.53- | |
| | | 3.42 (m, 2H), 1.35 (d, J = 6.6 Hz, 3H). | |
| 312 | 8, 9 | (400 MHz, DMSO-d6) δ 8.29 (s, 1H), 8.17 (s, 1H), 7.97 (t, | 420.9 |
| | | J = 6.1 Hz, 2H), 7.42 (td, J = 3.4, 1.8 Hz, 4H), 7.11 (dd, J = | |
| | | 9.7, 3.4 Hz, 2H), 6.99 (dd, J = 5.0, 3.5 Hz, 2H), 6.64 (s, | |
| | | 2H), 4.74 (d, J = 6.3 Hz, 4H), 4.62 (h, J = 6.1 Hz, 1H), 4.49 | |
| | | (dp, J = 12.1, 6.2 Hz, 1H), 3.47-3.25 (m, 4H), 3.28-3.20 | |
| | | (m, 1H), 1.37 (dd, J = 6.3, 2.4 Hz, 3H), 1.30 (dd, J = 6.3, | |
| | | 2.5 Hz, 3H). | |
| 313 | | (400 MHz, Chloroform-d) δ 7.03 (d, J = 3.4 Hz, 1H), 6.97 | 387.9 |
| | | (dd, J = 5.1, 3.4 Hz, 1H), 6.93 (s, 2H), 6.15 (s, 1H), 5.77 (s, | |
| | | 1H), 4.51 (t, J = 5.5 Hz, 2H), 3.93 (s, 1H), 3.00 (s, 1H), 2.64 | |
| | | (s, 1H). | |
| 314 | 8.9 | (300 MHz, Methanol-d4) δ 7.92 (s, 1H), 7.35 (dd, J = 5.1, | 401.9 |
| | | 1.2 Hz, 1H), 7.13 (dd, J = 3.4, 1.1 Hz, 2H), 7.02 (dd, J = | |
| | | 5.1, 3.5 Hz, 1H), 6.67 (s, 1H), 4.83 (s, 2H), 3.81 (h, J = 6.6 | |
| | | Hz, 1H), 3.67 (s, 3H), 3.23 (dd, J = 15.1, 6.5 Hz, 1H), 3.13 | |
| | | (dd, J = 15.1, 7.5 Hz, 1H), 1.34 (d, J = 6.6 Hz, 3H). | |
| 316 | 8, 9 | (300 MHz, Methanol-d4) δ 7.61 (d, J = 1.9 Hz, 1H), 7.34 | 401.9 |
| | | (dd, J = 5.1, 1.2 Hz, 1H), 7.13 (dd, J = 3.5, 1.2 Hz, 1H), | |
| | | 7.01 (dd, J = 5.1, 3.5 Hz, 1H), 6.67 (s, 1H), 6.46 (d, J = 2.0 | |
| | | Hz, 1H), 4.83 (s, 2H), 3.83 (s, 3H), 3.78 (q, J = 6.7 Hz, 1H), | |
| | | 3.23 (dd, J = 15.2, 6.4 Hz, 1H), 3.13 (dd, J = 15.2, 7.6 Hz, | |
| | | 1H), 1.33 (d, J = 6.6 Hz, 3H). | |
| 317 | 8, 9 | (400 MHz, Methanol-d4) δ 8.54 (s, 1H), 7.67 (d, J = 2.3 | 402.1 |
| | | Hz, 1H), 7.31 (dd, J = 5.1, 1.2 Hz, 1H), 7.09 (dt, J = 3.5, 1.1 | |
| | | Hz, 1H), 7.01-6.94 (m, 2H), 6.59 (s, 1H), 4.78 (d, J = 1.0 | |
| | | Hz, 2H), 3.98 (s, 3H), 3.83 (p, J = 6.7 Hz, 1H), 3.52-3.36 | |
| | | (m, 2H), 1.38 (d, J = 6.7 Hz, 3H). | |
| 319 | 8, 9 | (300 MHz, Methanol-d4) δ 8.53 (s, 1H), 7.77 (d, J = 2.4 | 387.9 |
| | | Hz, 1H), 7.31 (dd, J = 5.1, 1.2 Hz, 1H), 7.13-7.06 (m, | |
| | | 1H), 6.98 (dd, J = 4.9, 3.3 Hz, 2H), 6.61 (s, 1H), 4.79 (s, | |
| | | 2H), 3.81 (p, J = 6.5 Hz, 1H), 3.51-3.35 (m, 2H), 1.38 (d, | |
| | | J = 6.6 Hz, 3H). | |
| 320 | 8, 9 | -(400 MHz, Methanol-d4) δ 7.35-7.25 (m, 1H), 7.12- | 393.0 |
| | | 7.02 (m, 1H), 7.02-6.92 (m, 1H), 6.56 (s, 1H), 4.75 (s, | |
| | | 2H), 3.87 (s, 1H), 3.61-3.38 (m, 3H), 3.12-2.96 (m, | |
| | | 2H), 1.82-1.60 (m, 2H). | |
| 321 | 8, 9 | (400 MHz, Methanol-d4) δ 8.53 (s, 1H), 7.72 (q, J = 1.2 | 402.9 |
| | | Hz, 1H), 7.33 (dd, J = 5.0, 1.2 Hz, 1H), 7.11 (dd, J = 3.5, | |
| | | 1.2 Hz, 1H), 6.99 (dd, J = 5.1, 3.5 Hz, 1H), 6.61 (s, 1H), | |
| | | 4.76 (s, 2H), 4.59 (s, 1H), 3.99-3.90 (m, 1H), 3.59-3.41 | |
| | | (m, 2H), 2.24 (d, J = 1.3 Hz, 3H), 1.43 (d, J = 6.6 Hz, 3H). | |
| 322 | 8, 9 | (300 MHz, Methanol-d4) δ 8.53 (s, 1H), 7.77 (d, J = 2.4 | 402.9 |
| | | Hz, 1H), 7.31 (dd, J = 5.1, 1.2 Hz, 1H), 7.13-7.06 (m, | |
| | | 1H), 6.98 (dd, J = 4.9, 3.3 Hz, 2H), 6.61 (s, 1H), 4.79 (s, | |
| | | 2H), 3.81 (p, J = 6.5 Hz, 1H), 3.51-3.35 (m, 2H), 1.38 (d, | |
| | | J = 6.6 Hz, 3H). | |
| 327 | 8, 9 | -(400 MHz, Methanol-d4) δ 7.36-7.26 (m, 1H), 7.13- | 348.0 |
| | | 7.03 (m, 1H), 7.03-6.93 (m, 1H), 6.57 (s, 1H), 4.76 (s, | |
| | | 2H), 3.85 (s, 1H), 3.47-3.37 (m, 1H), 1.16 (d, J = 6.4 Hz, 3H). | |
| 328 | 8, 9 | (400 MHz, Methanol-d4) δ 8.53 (s, 1H), 8.09 (d, J = 1.4 | 387.9 |
| | | Hz, 1H), 7.65 (q, J = 1.5 Hz, 1H), 7.30 (dd, J = 5.1, 1.2 Hz, | |
| | | 1H), 7.09 (t, J = 2.2 Hz, 1H), 6.98 (dd, J = 5.1, 3.5 Hz, 1H), | |
| | | 6.87 (d, J = 1.8 Hz, 1H), 6.58 (d, J = 1.6 Hz, 1H), 3.76 (h, | |
| | | J = 6.7 Hz, 1H), 3.23 (dd, J = 15.3, 7.3 Hz, 1H), 1.32 (d, J = | |
| | | 6.6 Hz, 3H). | |
| 330 | 8, 9 | (400 MHz, Methanol-d4) δ 7.30 (td, J = 5.4, 1.3 Hz, 1H), | 377.9 |
| | | 7.08 (tt, J = 3.8, 1.9 Hz, 1H), 6.98 (dd, J = 5.1, 3.5 Hz, 1H), | |
| | | 6.60 (d, J = 16.1 Hz, 1H), 4.76 (d, J = 9.6 Hz, 2H), 4.58 (s, | |
| | | 1H), 3.96 (s, 1H), 3.80-3.68 (m, 1H), 3.57 (dd, J = 10.2, | |
| | | 3.8 Hz, 1H), 3.46-3.39 (m, 3H). | |
| 331 | 8, 9 | (400 MHz, Methanol-d4) δ 7.60 (dt, J = 1.8, 0.8 Hz, 1H), | 419.9 |
| | | 7.30 (dd, J = 5.1, 1.2 Hz, 1H), 7.12 (dt, J = 3.4, 1.0 Hz, 1H), | |
| | | 7.10-7.06 (m, 1H), 6.98 (dd, J = 5.1, 3.5 Hz, 1H), 6.59- | |
| | | 6.53 (m, 2H), 4.63-4.56 (m, 1H), 3.44 (ddt, J = 16.0, | |
| | | 12.2, 4.4 Hz, 2H), 3.25-3.12 (m, 1H), 1.40 (dd, J = 24.3, | |
| | | 6.4 Hz, 3H). | |
| 332 | 8, 9 | (400 MHz, Methanol-d4) δ 8.07 (dd, J = 1.6, 0.8 Hz, 1H), | 419.9 |
| | | 7.62 (t, J = 1.7 Hz, 1H), 7.30 (dd, J = 5.1, 1.2 Hz, 1H), 7.08 | |
| | | (dq, J = 2.2, 1.0 Hz, 1H), 6.98 (dd, J = 5.1, 3.4 Hz, 1H), | |
| | | 6.88 (dd, J = 1.9, 0.8 Hz, 1H), 6.55 (s, 1H), 4.76 (d, J = 1.1 | |
| | | Hz, 2H), 4.64 (ddt, J = 47.3, 11.1, 6.3 Hz, 1H), 3.45 (ddd, | |
| | | J = 20.0, 9.1, 4.7 Hz, 1H), 3.28 (d, J = 4.7 Hz, 1H), 3.02 (dd, | |
| | | J = 15.3, 8.8 Hz, 1H), 1.36 (dd, J = 24.4, 6.4 Hz, 3H). | |
| 333 | 8, 9 | (400 MHz, Methanol-d4) δ 8.53 (s, 1H), 7.63 (d, J = 2.2 | 433.9 |
| | | Hz, 1H), 7.30 (d, J = 5.1 Hz, 1H), 7.08 (d, J = 3.4 Hz, 1H), | |
| | | 7.01-6.94 (m, 1H), 6.90 (d, J = 2.2 Hz, 1H), 6.55 (s, 1H), | |
| | | 4.75 (s, 2H), 4.70 (td, J = 6.4, 3.9 Hz, 1H), 3.95 (s, 3H), | |
| | | 3.59 (ddt, J = 17.9, 8.8, 4.3 Hz, 1H), 3.46 (dd, J = 15.0, 4.5 | |
| | | Hz, 1H), 3.33-3.19 (m, 2H), 1.42 (dd, J = 24.2, 6.4 Hz, 3H). | |
| 334 | 8, 9 | (300 MHz, Methanol-d4) δ 7.62 (d, J = 1.9 Hz, 1H), 7.36 | 433.9 |
| | | (dd, J = 5.1, 1.3 Hz, 1H), 7.18-7.11 (m, 1H), 7.02 (dd, J = | |
| | | 5.1, 3.5 Hz, 1H), 6.70 (s, 1H), 6.50 (d, J = 2.0 Hz, 1H), 5.10 | |
| | | (tt, J = 6.9, 3.5 Hz, 0H), 3.99 (dddd, J = 21.8, 8.2, 5.3, 2.6 | |
| | | Hz, 1H), 3.85 (s, 3H), 3.41 (dd, J = 16.0, 5.4 Hz, 1H), 3.18 | |
| | | (dd, J = 15.9, 8.6 Hz, 1H), 1.38 (dd, J = 24.2, 6.5 Hz, 3H). | |
| 335 | 8, 9 | (400 MHz, Methanol-d4) δ 7.30 (dd, J = 5.1, 1.2 Hz, 1H), | 366.0 |
| | | 7.07 (dq, J = 3.3, 1.0 Hz, 1H), 6.97 (dd, J = 5.1, 3.5 Hz, | |
| | | 1H), 6.56 (s, 1H), 4.79-4.72 (m, 2H), 4.51-4.36 (m, | |
| | | 1H), 4.39-4.24 (m, 1H), 3.88 (s, 1H), 3.49 (dt, J = 18.7, | |
| | | 5.1 Hz, 1H). | |
| 336 | 6 | (400 MHz, Methanol-d4) δ 7.30 (dd, J = 5.1, 1.2 Hz, 1H), | 376.0 |
| | | 7.08 (dd, J = 3.4, 1.2 Hz, 1H), 6.97 (dd, J = 5.1, 3.5 Hz, | |
| | | 1H), 6.59 (s, 1H), 4.75 (s, 2H), 4.37 (dddd, J = 47.4, 25.9, | |
| | | 9.3, 5.0 Hz, 2H), 3.46 (dq, J = 18.1, 5.8 Hz, 1H), 3.12- | |
| | | 2.94 (m, 1H). | |
| 337 | 8 | (300 MHz, DMSO-d6): δ 8.33 (s, 2H), 7.87 (s, 1H), 7.33- | 424.0 |
| | | 7.17 (m, 2H), 7.04 (d, J = 8.2 Hz, 1H), 6.90 (m, 1H), 6.48 | |
| | | (s, 1H), 4.49 (d, J = 5.7 Hz, 2H), 3.86 (s, 3H), 3.25 (m, 1H), | |
| | | 3.10 (m, 1H), 1.22 (d, J = 6.9 Hz, 4H). | |
| 338 | 8 | (300 MHz, DMSO-d6): δ 8.49-8.42 (m, 3H), 8.15 (s, 1H), | 412.0 |
| | | 7.43 (m, 2H), 7.17 (t, J = 8.8 Hz, 2H), 6.53 (s, 1H), 4.53 (d, | |
| | | J = 4.0 Hz, 2H), 3.28 (m, 1H), 3.12 (m, 1H), 2.51 (m, | |
| | | 1H),1.24 (d, J = 6.5 Hz, 3H). | |
| 339 | 8 | (300 MHz, DMSO-d6): δ 8.33 (s, 2H), 8.19 (t, J = 6.4 Hz, | 419.0 |
| | | 1H), 7.86-7.78 (m, 2H), 7.57 (d, J = 8.1 Hz, 2H), 6.53 (s, | |
| | | 1H), 4.66 (d, J = 6.2 Hz, 2H), 3.25 (m, 1H), 3.11 (m, 1H), | |
| | | 1.23 (d, J = 6.3 Hz, 4H). | |
| 340 | 8 | (300 MHz, DMSO-d6): δ 8.30 (s, 3H), 7.97 (s, 1H), 7.36 | 412.0 |
| | | (m, 3H), 7.28-7.12 (m, 2H), 6.57 (s, 1H), 4.60 (d, J = 5.8 | |
| | | Hz, 2H), 3.25 (m, 1H), 3.10 (m, 1H), 1.76 (s, 1H), 1.22 (d, | |
| | | J = 6.7 Hz, 3H). | |
| 341 | 8 | (300 MHz, DMSO-d6): δ 8.67-8.57 (m, 3H), 7.68 (s, 1H), | 413.0 |
| | | 7.59 (q, J = 4.2 Hz, 2H), 6.59 (s, 1H), 4.69 (s, 2H), 3.31 (m, | |
| | | 1H), 3.13 (m, 1H), 2.51 (m, 1H) 1.22 (d, J = 6.4 Hz, 3H). | |
| 342 | 8 | (300 MHz, DMSO-d6): δ 8.94 (d, J = 2.7 Hz, 1H), 8.32 (s, | 413.0 |
| | | 1H), 7.72 (t, J = 6.0 Hz, 1H), 6.66 (s, 1H), 4.85-4.78 (m, | |
| | | 2H), 3.32 (m, 1H), 3.13 (m, 1H), 2.51(m, 1H), 1.23 (d, J = | |
| | | 6.3 Hz, 3H). | |
| 343 | 8 | (300 MHz, DMSO-d6): δ 8.41 (s, 2H), 8.10 (s, 1H), 7.26 | 442.0 |
| | | (m, 1H), 7.16 (m, 1H), 6.94 (m, 1H), 6.56 (s, 1H), 4.51 (s, | |
| | | 2H), 3.63 (s, 1H), 3.27 (m, 1H), 3.12 (m, 1H), 1.24 (d, J = | |
| | | 6.5 Hz, 3H). | |
| 347 | 8 | (300 MHz, DMSO-d6): δ 8.35 (s, 1H), 7.49-7.38 (m, 1H), | 387.0 |
| | | 7.14 (s, 1H), 7.04-6.91 (m, 1H), 6.77 (s, 1H), 4.75 (d, J = | |
| | | 5.9 Hz, 2H). | |
| 348 | 8 | (300 MHz, DMSO-d6): δ 8.38 (s, 3H), 8.04 (s, 1H), 7.73- | 425.0 |
| | | 7.62 (m, 1H), 6.94 (d, J = 7.3 Hz, 1H), 6.76-6.63 (m, 2H), | |
| | | 4.55 (s, 2H), 3.83 (s, 3H), 3.60 (s, 1H), 3.27 (dd, J = 15.1, | |
| | | 5.6 Hz, 1H), 3.11 (dd, J = 15.3, 8.3 Hz, 1H), 1.22 (d, J = 6.6 | |
| | | Hz, 3H) | |
| 349 | 8 | (300 MHz, DMSO-d6): δ 8.46-8.38 (m, 3H), 8.11 (d, J = | 478.0 |
| | | 6.7 Hz, 1H), 7.41 (tdd, J = 13.6, 7.8, 2.2 Hz, 4H), 6.47 (s, | |
| | | 1H), 4.62 (d, J = 5.3 Hz, 2H), 3.60 (s, 1H), 3.28 (dd, J = | |
| | | 15.2, 5.6 Hz, 1H), 3.11 (dd, J = 15.1, 8.2 Hz, 1H), 1.22 (d, | |
| | | J = 6.5 Hz, 3H). | |
| 350 | 8 | (300 MHz, DMSO-d6): δ 8.51-8.44 (m, 3H), 8.05 (s, 1H), | 430.0 |
| | | 7.45 (td, J = 8.7, 6.5 Hz, 1H), 7.27 (td, J = 9.7, 2.5 Hz, 1H), | |
| | | 7.07 (td, J = 8.6, 2.6 Hz, 1H), 6.58 (s, 1H), 3.62 (s, 1H), | |
| | | 3.29 (dd, J = 15.2, 5.6 Hz, 1H), 3.12 (dd, J = 15.2, 8.1 Hz, | |
| | | 1H), 1.23 (d, J = 6.3 Hz, 3H). | |
| 351 | 8 | (300 MHz, DMSO-d6): δ 8.26 (s, 2H), 7.98 (t, J = 6.0 Hz, | 448.0 |
| | | 1H), 7.38-7.28 (m, 1H), 7.25 (dd, J = 7.8, 2.1 Hz, 1H), | |
| | | 6.63 (s, 1H), 4.63 (d, J = 5.9 Hz, 2H), 3.22 (d, J = 5.8 Hz, | |
| | | 1H), 3.10 (dd, J = 15.2, 8.0 Hz, 1H), 1.23 (d, J = 6.5 Hz, 3H). | |
| 353 | 8 | (300 MHz, DMSO-d6): δ 8.29 (s, 3H), 8.07 (s, 1H), 7.00 (t, | 434.0 |
| | | J = 3.2 Hz, 2H), 6.68 (s, 1H), 4.69 (d, J = 5.7 Hz, 2H), 3.63 | |
| | | (s, 1H), 3.26 (dd, J = 15.1, 5.8 Hz, 1H), 3.11 (dd, J = 15.2, | |
| | | 7.9 Hz, 1H), 2.55 (s, 0H), 1.25 (d, J = 6.5 Hz, 3H). | |
| 354 | 8 | (300 MHz, DMSO-d6): δ 8.09 (d, J = 7.2 Hz, 1H), 7.49- | 373.0 |
| | | 7.24 (m, 1H), 7.10 (d, J = 3.4 Hz, 1H), 6.96 (t, J = 4.3 Hz, | |
| | | 1H), 6.63 (s, 1H), 4.71 (d, J = 5.1 Hz, 2H), 4.60 (s, 2H). | |
| 355 | 8 | (300 MHz, DMSO-d6): δ 8.04 (s, 1H), 7.40 (dd, J = 5.0, 1.3 | 387.0 |
| | | Hz, 1H), 7.11 (d, J = 3.5 Hz, 1H), 6.98 (dd, J = 5.1, 3.4 Hz, | |
| | | 1H), 6.64 (s, 1H), 4.73 (s, 2H), 1.48 (d, J = 6.6 Hz, 3H). | |
| 356 | | (300 MHz, DMSO-d6): δ 8.11 (s, 1H), 7.41 (dd, J = 5.0, 1.3 | 401.0 |
| | | Hz, 1H), 7.14-7.08 (m, 1H), 6.98 (dd, J = 5.1, 3.4 Hz, | |
| | | 1H), 6.67 (s, 1H), 4.80-4.56 (m, 3H), 1.52 (d, J = 6.6 Hz, 3H). | |
| 357 | | (400 MHz, Methanol-d4) δ 8.52 (s, 1H), 7.45 (dd, J = 1.9, | 376.9 |
| | | 0.9 Hz, 1H), 6.68 (s, 1H), 6.40-6.32 (m, 2H), 4.57 (s, 2H), | |
| | | 3.73 (q, J = 6.6 Hz, 1H), 3.17 (dd, J = 6.4, 2.4 Hz, 2H), 3.14 | |
| | | (s, 3H), 3.05 (s, 3H), 1.38 (d, J = 6.7 Hz, 3H). | |
| 358 | | (400 MHz, Methanol-d4) δ 8.53 (s, 1H), 7.45 (d, J = 1.8 | 362.9 |
| | | Hz, 1H), 6.67 (s, 1H), 6.40-6.31 (m, 2H), 4.57 (s, 2H), | |
| | | 3.76 (p, J = 6.6 Hz, 1H), 3.53 (dd, J = 6.5, 1.6 Hz, 2H), 3.00 | |
| | | (s, 3H), 1.34 (d, J = 6.7 Hz, 3H). | |
| 359 | 8 | (300 MHz, DMSO-d6): 87.93 (t, J = 5.9 Hz, 1H), 7.16- | 442 |
| | | 7.05 (m, 2H), 6.92 (td, J = 6.3, 3.0 Hz, 1H), 6.57 (s, 1H), | |
| | | 4.60 (d, J = 5.9 Hz, 2H), 3.84 (s, 3H), 3.62 (s, 1H), 3.24 | |
| | | (dd, J = 15.1, 5.9 Hz, 1H), 3.09 (dd, J = 15.0, 7.8 Hz, 1H), | |
| | | 1.23 (d, J = 6.5 Hz, 3H). | |
| 360 | Example 26 | (300 MHz, DMSO-d6): δ 8.17 (s, 1H), 7.39 (d, J = 5.0 Hz, | 387 |
| | | 1H), 7.10 (d, J = 3.4 Hz, 1H), 6.97 (t, J = 4.3 Hz, 1H), 6.67 | |
| | | (s, 1H), 4.71 (s, 2H), 4.58 (s, 2H), 4.29 (s, 3H). | |
| 361 | 8 | (300 MHz, DMSO-d6): δ 8.47 (s, 3H), 8.23 (s, 1H), 8.14 (d, | 425.0 |
| | | J = 5.4 Hz, 1H), 7.08-7.00 (m, 1H), 6.87 (s, 1H), 6.51 (s, | |
| | | 1H), 4.58 (d, J = 4.1 Hz, 2H), 3.85 (s, 3H), 3.63 (s, 1H), | |
| | | 3.29 (m, 1H), 3.13 (m, 1H), 1.24 (d, J = 6.4 Hz, 3H). | |
| 362 | 8 | (300 MHz, DMSO-d6): 87.28 (d, J = 7.7 Hz, 1H), 7.07 (s, | 424.0 |
| | | 2H), 6.88-6.79 (m, 1H), 6.52 (s, 1H), 4.52 (d, J = 5.5 Hz, | |
| | | 2H), 3.74 (s, 3H), 3.63 (s, 1H), 3.26 (m, 1H), 3.11 (m, 1H), | |
| | | 1.24 (d, J = 6.5 Hz, 3H). | |
| 363 | | (400 MHz, Methanol-d4) δ 7.34-7.24 (m, 1H), 7.12- | 362.0 |
| | | 7.02 (m, 1H), 7.02-6.92 (m, 1H), 6.57-6.43 (m, 2H), | |
| | | 4.72 (s, 2H), 3.52-3.42 (m, 1H), 3.22-3.12 (m, 1H), | |
| | | 2.01-1.42 (m, 8H). | |
| 364 | | (400 MHz, Methanol-d4) δ 9.10 (s, 1H), 8.53 (s, 1H), 7.46 | 374.1 |
| | | (dd, J = 1.8, 0.9 Hz, 1H), 6.71 (s, 1H), 6.41-6.34 (m, 2H), | |
| | | 4.58 (s, 3H), 3.81 (q, J = 6.6 Hz, 1H), 3.60-3.41 (m, 2H), | |
| | | 1.36 (d, J = 6.6 Hz, 3H). | |
| 365 | | (400 MHz, Methanol-d4) δ 8.54 (s, 1H), 7.98 (s, 1H), 7.77 | 416.0 |
| | | (s, 1H), 7.46 (s, 1H), 6.71 (s, 1H), 6.41-6.32 (m, 2H), | |
| | | 4.57 (d, J = 6.6 Hz, 3H), 3.90 (q, J = 6.6 Hz, 1H), 3.57 (d, | |
| | | J = 6.6 Hz, 2H), 1.41 (d, J = 6.6 Hz, 3H), 1.36-1.20 (m, 1H), | |
| | | 0.87 (s, 1H). | |
| 366 | | (400 MHz, Methanol-d4) δ 8.10 (s, 1H), 7.45 (d, J = 1.9 | 398.0 |
| | | Hz, 1H), 6.70 (s, 1H), 6.40-6.32 (m,2H), 4.57 (s, 2H), | |
| | | 3.49 (p, J = 6.5 Hz, 1H), 3.43-3.32 (m, 2H), 1.20 (d, J = | |
| | | 6.4 Hz, 3H). | |
| 367 | 13 | 1 H NMR (400 MHz, Methanol-d4) δ 8.49 (s, 1H), 7.31 | 411.9 |
| | | (dd, J = 5.1, 1.2 Hz, 1H), 7.09 (dd, J = 3.5, 1.2 Hz, 1H), | |
| | | 6.99 (dd, J = 5.1, 3.5 Hz, 1H), 6.63 (s, 1H), 4.79 (s, 2H), | |
| | | 4.09 (q, J = 8.5 Hz, 1H), 3.90 (q, J = 9.1 Hz, 1H), 2.44- | |
| | | 2.20 (m, 3H), 2.15 (q, J = 9.7 Hz, 1H). | |
| 368 | | (400 MHz, Methanol-d4) δ 7.30 (d, J = 5.1 Hz, 1H), 7.05 | 426.8 |
| | | (d, J = 3.5 Hz, 1H), 6.97 (dd, J = 5.1, 3.5 Hz, 1H), 6.53 (s, | |
| | | 1H), 4.74 (s, 2H), 4.43 (d, J = 6.7 Hz, 2H), 2.79 (p, J = 6.9, | |
| | | 6.2 Hz, 1H), 2.13 (q, J = 9.6, 8.5 Hz, 2H), 2.05-1.87 (m, 4H). | |
| 369 | | (400 MHz, Methanol-d4) δ 8.41 (s, 1H), 7.31 (dd, J = 5.1, | 461.85 |
| | | 1.2 Hz, 1H), 7.08 (dq, J = 3.4, 1.1 Hz, 1H), 6.98 (dd, J = | |
| | | 5.1, 3.5 Hz, 1H), 6.63 (d, J = 0.8 Hz, 1H), 4.76 (s, 2H), 4.00 | |
| | | (q, J = 9.4 Hz, 1H), 3.73 (dt, J = 11.7, 9.2 Hz, 1H), 2.73 | |
| | | (dddd, J = 43.5, 21.3, 15.2, 6.9 Hz, 2H), 2.61-2.49 (m, | |
| | | 1H), 2.39-2.20 (m, 1H). | |
| 373 | | (400 MHz, Methanol-d4) δ 7.30 (dd, J = 5.1, 1.2 Hz, 1H), | 427.8 |
| | | 7.07 (dq, J = 3.3, 1.0 Hz, 1H), 6.97 (dd, J = 5.1, 3.5 Hz, | |
| | | 1H), 6.59 (s, 1H), 4.75 (s, 2H), 4.28 (t, J = 8.5 Hz, 1H), 4.17 | |
| | | (dd, J = 8.8, 6.5 Hz, 1H), 3.99 (dd, J = 8.8, 7.1 Hz, 1H), | |
| | | 3.87 (q, J = 6.2 Hz, 1H), 3.63 (dt, J = 8.5, 6.6 Hz, 2H). | |
| 374 | | (400 MHz, Methanol-d4) δ 7.30 (dd, J = 5.1, 1.3 Hz, 1H), | 427.8 |
| | | 7.07 (dt, J = 3.5, 1.1 Hz, 1H), 6.97 (dd, J = 5.1, 3.4 Hz, 1H), | |
| | | 6.60 (d, J = 1.1 Hz, 1H), 4.29 (t, J = 8.5 Hz, 1H), 4.17 (dd, | |
| | | J = 8.9, 6.5 Hz, 1H), 3.99 (dd, J = 8.8, 7.1 Hz, 1H), 3.89 (q, | |
| | | J = 6.0 Hz, 1H), 3.65 (dt, J = 9.3, 5.6 Hz, 2H). | |
| 380 | 13 | (400 MHz, Methanol-d4) δ 7.30 (dd, J = 5.1, 1.2 Hz, 1H), | 475.9 |
| | | 7.07 (dq, J = 3.4, 1.1 Hz, 1H), 6.97 (dd, J = 5.1, 3.5 Hz, | |
| | | 1H), 6.61 (s, 1H), 4.80-4.77 (m, 2H), 3.26-3.14 (m, | |
| | | 2H), 2.37-2.13 (m, 3H), 2.10-1.88 (m, 2H), 1.66 (q, J = | |
| | | 12.3, 11.6 Hz, 1H). | |
| 388 | | (400 MHz, Methanol-d4) δ 8.41 (s, 1H), 7.31 (dd, J = 5.1, | 461.85 |
| | | 1.2 Hz, 1H), 7.08 (dq, J = 3.4, 1.1 Hz, 1H), 6.98 (dd, J = | |
| | | 5.1, 3.5 Hz, 1H), 6.63 (d, J = 0.8 Hz, 1H), 4.76 (s, 2H), 4.00 | |
| | | (q, J = 9.4 Hz, 1H), 3.73 (dt, J = 11.7, 9.2 Hz, 1H), 2.73 | |
| | | (dddd, J = 43.5, 21.3, 15.2, 6.9 Hz, 2H), 2.61-2.49 (m, | |
| | | 1H), 2.39-2.20 (m, 1H). | |
| 404 | 6 | (400 MHz, Methanol-d4) 7.29 (dd, J = 5.1, 1.3 Hz, 1H), | 320 |
| | | 7.07 (dt, J = 3.5, 1.1 Hz, 1H), 6.96 (dd, J = 5.1, 3.4 Hz, | |
| | | 1H), 6.09 (d, J = 1.8 Hz, 1H), 4.77-4.69 (m, 2H), 3.43 | |
| | | (q, J = 6.5 Hz, 1H), 2.98-2.83 (m, 2H), 2.13 (s, 3H), | |
| | | 1.18 (d, J = 6.5 Hz, 3H). | |

### Example 34: ATXN3 Quantitative Splicing Assay

Human neuroblastoma SK-N-MC cells were plated in 384-well plates at 20,000 cells/well. Twenty-four hours after plating, cells were treated with compounds for 24 h at appropriate concentrations ranging from 30 μM to 0.6 nM (0.300 DMSO). Treated cells were lysed in 15 μL of lysis buffer, and cDNA was synthesized using the Fast Advanced Cells-to-Ct kit. Two L of each cDNA was used in qPCR reactions to confirm the exon 4 skipped transcripts of ATXN3. A second set of primers/probe E4E5 was used to detect the transcripts containing exon 4. The third set of primers/probe E8E9 was used to detect total gene level of ATXN3. The qPCR reactions were prepared in 384-well plates in 10 μL volume, using TaqMan™ Fast Advanced Master Mix with primers and probes shown in the table below. Reactions were run in a Quant Studio 6 qPCR instrument with default settings.

The primers and probes are listed below in Table 4.

TABLE 4

| Target | | | |
|---|---|---|---|
| Sequence | Forward Primer | Probe | Reverse Primer |
| ATXN3 | SEQ ID NO: 2 | SEQ ID NO: 3 | SEQ ID NO: 4 |
| E4skpping- | 5′ | 5′ TTCTCTATTCAGAAAT | 5′ |
| FAM | GCAGCCTTCTGGAAA | GAAAGATCATT 3′ | CTGGACCCGTCAAGA |
| | TATGG 3′ | | GAGAA 3′ |
| ATXN3 | SEQ ID NO: 5 | SEQ ID NO: 6 | SEQ ID NO: 7 |
| E4E5-Cy5 | 5′ | 5′ | 5′ |
| | TGTTCAACAGTCCAG | AGGCTCAGGATCGAT | ACCCGTCAAGAGAGA |
| | AGTATCAG 3′ | CCTATAAATGAAAGA | ATTCAAG 3′ |
| | | 3′ | |
| ATXN3 | SEQ ID NO: 8 | SEQ ID NO: 9 | SEQ ID NO: 10 |
| E8E9- | 5′ | 5′ | 5′ |
| total-FAM | GATGAGGAGGATTTG | ATGTTTCTGGAACTAC | CCTGATGTCTGTGTCA |
| | CAGAGG 3′ | CTTGCATACTTAGCTG | TATCTTGA 3′ |
| | | 3′ | |
| TBP-YAK | SEQ ID NO: 11 | SEQ ID NO: 12 | SEQ ID NO: 13 |
| (endogenous | 5′ | 5′ | 5′ |
| control) | TCGGAGAGTTCTGGG | CCGCAGCTGCAAAAT | AAGTGCAATGGTCTTT |
| | ATT 3′ | ATTGTATCCACA 3′ | AGGT 3′ |

### Example 35: ATXN3 Total Protein Assay

Human neuroblastoma SK-N-MC cells were seeded at 10,000 cells/well in 384 well plates one day prior to compound treatment. The concentrations of compounds were tested at appropriate doses ranging from 30 μM to 0.6 nM. After incubation for 48 hours, the cells were lysed with 25 μL of lysis buffer containing protease inhibitors, and total ATXN3 protein levels were assessed by Mesoscale Discovery (MSD) assay developed with one pair of anti-ATXN3 antibodies. The capture and detect antibodies were raised in mouse and rabbit respectively. Anti-rabbit MSD-ST antibody was used for secondary antibody.

ATXN3 recombinant protein was used for standards. The readouts were captured with 35 μL of MSD read buffer and multi-array 384-well high binding plates.

One plate replica was carried out for parallel viability testing by CellTiter Glo® 2.0 with a seeding density of 4,000 cells/well. Compounds were incubated for 48 hours. The viability readouts were carried out by Envision according to the manufacturer's instructions.

### Example 36: ATXN3 Total Protein Assay

To monitor ATXN3 protein levels by luminescence, a Kelly-ATXN3-HiBiT cell line with homozygous knock-in of HiBiT at the C-terminus of the endogenous ATXN3 gene was used (knock-in of the HiBiT-tag coding sequence SEQ ID NO. 14: 5′GTGAGCGGCTGGCGGCTGTTCAAGAAGATTAGC3′ into exon 11 of ATXN3-position 1114 of ATXN3 RefSeq NM_004993.6). Manipulations of the Kelly-ATXN3-HiBiT cell line that result in downregulation of the ATXN3-HiBiT protein (through modulation of ATXN3-HiBiT pre-mRNA splicing) can be followed by monitoring the luminescence signal of the HiBiT tag using the Nano Glo HiBiT Lytic Detection System (Promega, #N3050). Kelly-ATXN3-HiBiT cells were maintained in in RPMI 1640 w/Glutamax (Gibco, #61870010), 10% FBS (Sigma Aldrich, #F8687), 1% Penicillin/Streptomycin (Gibco, #15140-122) at 37° C., 5% CO₂. To assess HiBiT-luminescence signal upon compound treatment, 2000 Kelly-ATXN3-HiBiT cells were seeded per well in 384 plate format. Compounds were added 24 h after seeding. ATXN3-HiBiT-luminescence signal was read out 48 h after addition of the compound using the Nano Glo HiBiT Lytic Detection System (Promega, #N3050) according to the manufacturer's instructions. To control for cell viability, an identically prepared sister plate was read out with the CellTiter-Glo® 2.0 Assay (Promega, #G9243) according to the manufacturer's instructions. Fold changes of ATXN3-HiBiT-luminescence and of CellTiter-Glo viability signals were calculated relative to a DMSO control condition.

Compounds were tested as outlined in Examples 34-36 above and the results are shown below in Table 5.

TABLE 5

| Compound | ATXN3 E4E5 | ATXN3 Protein | ATXN3 HiBit |
|---|---|---|---|
| # | IC50 (nM) | IC50 (nM) | EC50 (nM) |
| 1 | E | E | |
| 2 | D | D | |
| 3 | C | C | |
| 4 | D | C | |
| 5 | E | E | |
| 6 | E | E | |
| 7 | C | C | |
| 8 | C | C | |
| 9 | C | C | |
| 10 | C | C | |
| 11 | B | B | A |
| 12 | C | C | |
| 13 | B | B | B |
| 14 | B | B | A |
| 15 | C | C | |
| 16 | A | A | A |
| 17 | A | A | A |
| 18 | E | D | D |
| 19 | B | B | |
| 20 | A | A | A |
| 21 | A | A | A |
| 22 | A | A | A |
| 23 | A | A | A |
| 24 | B | B | |
| 25 | C | C | B |
| 26 | B | B | C |
| 27 | C | C | C |
| 28 | B | B | |
| 29 | B | B | |
| 30 | C | C | |
| 31 | B | B | B |
| 32 | C | C | |
| 34 | A | A | A |
| 35 | B | B | B |
| 36 | C | C | C |
| 37 | A | A | A |
| 38 | B | A | |
| 39 | A | A | |
| 40 | C | C | C |
| 41 | A | A | A |
| 42 | C | C | |
| 43 | A | A | A |
| 44 | B | B | B |
| 45 | B | B | A |
| 47 | B | B | |
| 48 | B | B | B |
| 49 | B | B | |
| 50 | A | A | A |
| 51 | B | B | B |
| 52 | A | B | A |
| 53 | B | B | B |
| 54 | B | A | |
| 55 | A | A | |
| 56 | A | A | |
| 57 | B | B | B |
| 58 | A | A | A |
| 59 | B | B | A |
| 60 | B | B | B |
| 61 | A | A | A |
| 62 | B | B | A |
| 63 | A | A | A |
| 64 | A | A | A |
| 65 | B | B | B |
| 66 | A | A | A |
| 67 | | | B |
| 68 | | | C |
| 69 | | | A |
| 70 | | | E |
| 73 | | | E |
| 77 | A | A | A |
| 85 | B | B | B |
| 86 | | | C |
| 87 | | | B |
| 88 | A | A | B |
| 90 | A | A | A |
| 91 | C | C | C |
| 92 | B | B | B |
| 93 | C | C | C |
| 94 | B | B | B |
| 95 | C | C | C |
| 96 | B | B | B |
| 97 | C | C | C |
| 98 | C | C | C |
| 99 | B | B | C |
| 100 | C | D | E |
| 103 | B | B | B |
| 104 | B | | |
| 105 | B | C | C |
| 106 | B | B | B |
| 107 | B | C | B |
| 108 | B | B | B |
| 109 | A | A | B |
| 110 | B | B | B |
| 111 | B | B | B |
| 112 | B | B | A |
| 113 | B | B | B |
| 114 | C | C | C |
| 115 | B | B | B |
| 116 | B | B | C |
| 117 | A | A | A |
| 118 | A | A | A |
| 119 | A | A | B |
| 120 | B | B | C |
| 121 | B | B | B |
| 122 | A | A | A |
| 123 | A | A | A |
| 124 | C | C | C |
| 125 | A | A | A |
| 126 | B | B | B |
| 131 | B | A | A |
| 132 | C | B | C |
| 133 | C | C | C |
| 134 | C | C | C |
| 139 | C | C | C |
| 140 | B | B | B |
| 142 | B | B | B |
| 144 | | | E |
| 145 | B | B | B |
| 146 | | | A |
| 147 | | | A |
| 148 | A | A | A |
| 149 | B | B | B |
| 150 | A | A | A |
| 151 | A | B | A |
| 152 | B | B | C |
| 153 | E | D | E |
| 154 | A | A | A |
| 155 | | | C |
| 157 | A | A | A |
| 160 | C | B | B |
| 162 | A | A | B |
| 164 | D | D | C |
| 167 | E | E | E |
| 169 | C | B | B |
| 170 | A | | |
| 172 | C | | |
| 174 | A | | |
| 176 | C | | |
| 178 | C | C | C |
| 180 | B | B | B |
| 181 | C | C | C |
| 182 | | | A |
| 183 | | | B |
| 184 | | | B |
| 187 | A | | A |
| 188 | B | B | A |
| 189 | B | B | B |
| 190 | C | C | C |
| 192 | B | B | B |
| 193 | C | C | C |
| 194 | C | C | B |
| 196 | A | A | A |
| 197 | A | | A |
| 213 | C | C | C |
| 214 | C | C | C |
| 215 | E | E | E |
| 216 | B | B | B |
| 219 | | | A |
| 221 | A | A | A |
| 222 | B | A | A |
| 223 | | | B |
| 224 | | | C |
| 226 | C | C | C |
| 227 | C | C | C |
| 229 | | | A |
| 230 | | | A |
| 231 | | | A |
| 232 | | | B |
| 233 | | | A |
| 234 | | | A |
| 235 | | | C |
| 237 | E | D | E |
| 238 | E | E | E |
| 239 | B | B | B |
| 240 | C | C | C |
| 242 | B | B | B |
| 243 | C | B | C |
| 244 | | | A |
| 246 | | | B |
| 248 | | | A |
| 249 | | | A |
| 250 | | | A |
| 252 | E | D | C |
| 253 | C | C | E |
| 254 | C | C | C |
| 256 | E | D | E |
| 257 | E | E | E |
| 260 | E | C | E |
| 262 | B | B | B |
| 263 | C | C | C |
| 264 | | | A |
| 265 | | | A |
| 266 | | | A |
| 267 | | | B |
| 268 | | | A |
| 269 | | | A |
| 270 | | | A |
| 271 | | | B |
| 273 | | | B |
| 274 | | | B |
| 275 | C | C | C |
| 276 | C | C | C |
| 277 | C | C | C |
| 278 | B | | |
| 279 | | | B |
| 280 | | | E |
| 281 | | | C |
| 282 | | | A |
| 283 | | | A |
| 284 | | | A |
| 285 | B | A | A |
| 286 | | | A |
| 287 | | | A |
| 288 | E | E | E |
| 290 | C | | |
| 291 | B | | |
| 292 | | | A |
| 293 | | | A |
| 295 | C | C | B |
| 296 | | | A |
| 297 | | | A |
| 298 | | | A |
| 299 | | | A |
| 300 | | | A |
| 301 | | | A |
| 302 | | | A |
| 304 | A | A | A |
| 305 | | | A |
| 310 | B | B | B |
| 311 | C | D | C |
| 312 | B | B | A |
| 313 | E | D | E |
| 314 | E | E | E |
| 316 | E | E | E |
| 317 | E | E | C |
| 319 | C | C | C |
| 320 | | | A |
| 321 | C | C | C |
| 322 | C | C | C |
| 327 | | | A |
| 328 | E | E | E |
| 330 | | | A |
| 331 | C | C | C |
| 332 | C | C | C |
| 333 | C | C | D |
| 334 | E | E | E |
| 335 | | | A |
| 336 | | | A |
| 337 | | | E |
| 338 | C | C | C |
| 339 | | | E |
| 340 | C | C | C |
| 341 | | | D |
| 342 | | | B |
| 343 | | | C |
| 347 | | | E |
| 348 | | | E |
| 349 | | | E |
| 350 | C | | C |
| 351 | | | E |
| 353 | C | | C |
| 354 | D | | D |
| 355 | | | D |
| 356 | | | E |
| 357 | E | | |
| 358 | E | | |
| 359 | E | | |
| 360 | D | | |
| 361 | E | | |
| 362 | D | | |
| 363 | C | | |
| 364 | C | | |
| 365 | E | | |
| 366 | E | | |
| 367 | B | | |
| 368 | C | | |
| 369 | D | | |
| 373 | B | | |
| 374 | E | | |
| 380 | B | | |
| 385 | A | | |
| 388 | B | | |
| 404 | B | | |

ATXN3 E4E5 IC50 (nM): 0.01 ≤ A ≤ 100; 101 ≤ B ≤ 500; 501 ≤ C ≤ 5000; 5001 ≤ D ≤ 10000; 10001 ≤ E ≤ 40,000.
ATXN3 Protein IC50 (nM): 0.01 ≤ A ≤ 100; 101 ≤ B ≤ 500; 501 ≤ C ≤ 5000; 5001 ≤ D ≤ 10000; 10001 ≤ E ≤ 40,000.
ATXN3 HiBit EC50 (nM): 0.01 ≤ A ≤ 100; 101 ≤ B ≤ 500; 501 ≤ C ≤ 5000; 5001 ≤ D ≤ 10000; 10001 ≤ E ≤ 40,000.

1. A compound of Formula (I), or a pharmaceutically acceptable salt thereof: 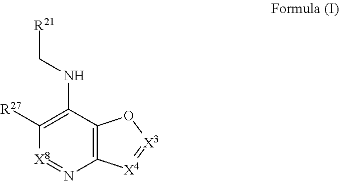
wherein,
X³ is selected from the group consisting of N, and CR²³;
X⁴ is selected from the group consisting of N, and CR²⁴;
X⁸ is CR²⁸ or N;
R²¹ is selected from the group consisting of phenyl, 5-6 membered heteroaryl, and 5-6 membered heterocycloalkyl, each of which is unsubstituted or substituted with 1, 2, 3 or 4, independently selected R^{1A} groups; each R^{1A} is independently selected from halo, CN, NO₂, C₁₋₆ alkyl, C₂₋₆ alkenyl, C₂₋₆ alkynyl, C₁₋₆ haloalkyl, C₁₋₄ haloalkoxy, C₁₋₆ alkoxy, —C(═O)OH, —C(═O)C₁₋₆ alkyl, —C(═O)C₁₋₆ haloalkyl, and —C(═O)C₁₋₆ alkoxy;
R²³ is selected from the group consisting of H, azido, halo, —CN, —NO₂, C₁₋₆ alkyl, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, 4-10 membered heterocycloalkyl, —(C₁₋₆ alkylene)-C₃₋₁₀ cycloalkyl, —(C₁₋₆ alkylene)-4-10 membered heterocycloalkyl, —(C₁₋₆ heteroalkylene)-C₃₋₁₀ cycloalkyl, —(C₁₋₆ heteroalkylene)-4-10 membered heterocycloalkyl, —(C₁₋₆ alkylene)-C₆₋₁₀ aryl, —(C₁₋₆ alkylene)-5-10 membered heteroaryl, —(C₁₋₆ heteroalkylene)-C₆₋₁₀ aryl, —(C₁₋₆ heteroalkylene)-5-10 membered heteroaryl, —OR^{a3}, —SR^{a3}, —C(═O)R^{b3}, —C(═O)OR^{b3}, —NR^{c3}R^{d3}, —C(═O)NR^{c3}R^{d3}, —OC(═O)NR^{c3}R^{d3}, —NR^{c3}C(═O)R^{b3}, —NR^{c3}C(═O)OR^{b3}, —NR^{c3}C(═O)NR^{c3}R^{d3}, —NR^{c3}S(═O)₂R^{b3}, —NR^{c3}S(═O)₂NR^{c3}R^{d3}, —S(O)NR^{c3}R^{d3}, and —S(O)₂NR^{c3}R^{d3}, wherein the C₁₋₆ alkyl, C₁₋₆ alkylene, C₁₋₆ heteroalkylene, C₂₋₆ alkenyl, C₂₋₆ alkynyl, C₁₋₆ heteroalkyl, C₃₋₁₀ cycloalkyl, C₆₋₁₀ aryl, 5-10 membered heteroaryl, and 4-10 membered heterocycloalkyl are each unsubstituted or substituted with 1, 2, 3, or 4 independently selected R²⁰ groups;
R²⁴ is selected from the group consisting of —C≡CH, H, azido, halo, —CN, —NO₂, C₁₋₆ alkyl, C₂₋₆ alkenyl, C₁₋₆ heteroalkyl, C₃₋₁₀ cycloalkyl, C₆₋₁₀ aryl, 5-10 membered heteroaryl, 4-10 membered heterocycloalkyl, —OR^{a4}, —C(═O)R^{b4}, —C(═O)OR^{b4}, —NR^{c4}R^{d4}, —C(═O)NR^{c4}R^{d4}, —OC(═O)NR^{c4}R^{d4}, —NR⁴C(═O)R^{b4}, —NR^{c4}C(═O)OR^{b4}, —NR^{c4}C(═O)NR^{c4}R^{d4}, —NR^{c4}S(═O)₂R^{b4}, —NR^{c4}S(═O)₂NR^{c4}R^{d4}, —S(O)NR^{c4}R^{d4}, and —S(O)₂NR^{c4}R^{d4}, wherein the C₁₋₆ alkyl, C₂₋₆ alkenyl, C₁₋₆ heteroalkyl, C₃₋₁₀ cycloalkyl, C₆₋₁₀ aryl, 5-10 membered heteroaryl, and 4-10 membered heterocycloalkyl, are each unsubstituted or substituted with 1, 2, 3, or 4 independently selected R^{20d} groups;
each R^{20d} is independently selected from the group consisting of —OH, —SH, —CN, —NO₂, halogen, oxo, amino, carbamyl, carbamoyl, C₁₋₄ alkyl, C₂₋₄ alkenyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino;
R²⁷ is selected from the group consisting of H, azido, halo, C₁₋₆ alkyl, C₂₋₆ alkenyl, C₂₋₆ alkynyl, C₁₋₆ heteroalkyl, —CN, —NO₂, —OR^{a7}, —C(═O)R^{b7}, —C(═O)OR^{b7}, —NR^{c7}R^{d7}, —C(═O)NR^{c7}R^{d7}, —OC(═O)NR^{c7}R^{d7}, —NR^{c7}C(═O)R^{b7}, —NR^{c7}C(═O)OR^{b7}, —NR^{c7}C(═O)NR^{c7}R^{d7}, —NR^{c7}S(═O)₂R^{b7}, and —NR^{c7}S(═O)₂NR^{c7}R^{d7}, wherein the C₁₋₆ alkyl, C₁₋₆ heteroalkyl, C₂₋₆ alkenyl, and C₂₋₆ alkynyl are each unsubstituted or substituted with 1, 2, 3, or 4 independently selected R²⁰ groups;
R²⁸ is selected from the group consisting of H, oxo, azido, halo, C₁₋₆ alkyl, C₂₋₆ alkenyl, C₂₋₆ alkynyl, C₁₋₆ heteroalkyl, —CN, —NO₂, —OR^{a8}, —C(═O)R^{b8}, —C(═O)OR^{b8}, —NR^{c8}R^{d8}, —C(═O)NR^{c8}R^{d8}, —OC(═O)NR^{c8}R^{d8}, —NR^{c8}C(═O)R, —NR^{c8}C(═O)OR^{d8}, —NR^{c8}C(═O)NR^{c8}R^{d8}, —NR^{c8}S(═O)₂R^{b8}, and —NR^{c8}S(═O)₂NR^{c8}R^{d8}, wherein the C₁₋₆ alkyl, C₁₋₆ heteroalkyl, C₂₋₆ alkenyl, and C₂₋₆ alkynyl are each unsubstituted or substituted with 1, 2, 3, or 4 independently selected R²⁰ groups;
each R^{a3}, R^{b3}, R^{c3}, R^{d3}, R^{a4}, R^{b4}, R^{c4}, R^{d4}, R^{a7}, R^{b7}, R^{c7}, R^{d7}, R^{a8}, R^{b8}, R^{c8}, and R^{d8} is independently selected from the group consisting of H, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, and 4-10 membered heterocycloalkyl each of which is unsubstituted or substituted with 1, 2, 3, or 4 independently selected R²⁰ groups;
or R^{c3} and R^{d3} together with the N atom to which they are connected, come together to form a 5-10 membered heteroaryl or a 4-10 membered heterocycloalkyl ring, each of which is unsubstituted or substituted with 1, 2, 3, or 4 independently selected R²⁰ groups;
or R^{c4} and R^{d4} together with the N atom to which they are connected, come together to form a 5-10 membered heteroaryl or a 4-10 membered heterocycloalkyl ring, each of which is unsubstituted or substituted with 1, 2, 3, or 4 independently selected R²⁰ groups; and
each R²⁰ is independently selected from the group consisting of —OH, —SH, —CN, —NO₂, halo, oxo, amino, carbamyl, carbamoyl, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₆₋₁₀ aryl, 5-6 membered heteroaryl, 4-6 membered heterocycloalkyl, C₁₋₄ alkylamino, di(C₁₋₄ alkyl)amino, C₁₋₄ alkylcarbamyl, di(C₁₋₄ alkyl)carbamyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

2. The compound of claim 1, or pharmaceutically acceptable salt thereof, wherein R²¹ is selected from the group consisting of phenyl and 5-6 membered heteroaryl, each of which is unsubstituted or substituted with 1, 2, 3 or 4, independently selected R^{1A} groups; each R^{1A} is independently selected from halo, CN, NO₂, C₁₋₆ alkyl, C₂₋₆ alkenyl, C₂₋₆ alkynyl, C₁₋₆ haloalkyl, C₁₋₆ alkoxy, —C(═O)OH, —C(═O)C₁₋₆ alkyl, —C(═O)C₁₋₆ haloalkyl, and —C(═O)C₁₋₆ alkoxy.

3. The compound of claim 2, or pharmaceutically acceptable salt thereof, wherein R²¹ is selected from the group consisting of

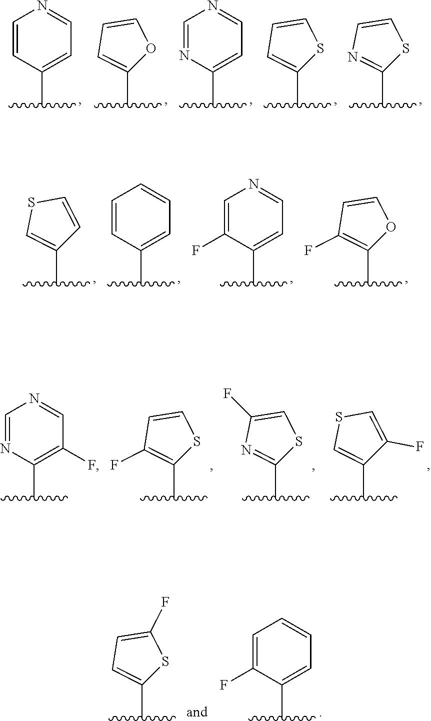

4. The compound of claim 1, or pharmaceutically acceptable salt thereof, wherein X³ is CH.

5. The compound of claim 1, or pharmaceutically acceptable salt thereof, wherein X³ is CR²³.

6. The compound of claim 5, or pharmaceutically acceptable salt thereof, wherein R²³ is substituted or unsubstituted C₁₋₆ alkyl or substituted or unsubstituted C₁₋₆ heteroalkyl.

7. The compound of claim 6, or a pharmaceutically acceptable salt thereof, wherein X³ is CCH₂CH(NH₂)CH₃, CCH₂CH(NH₂)CH₂OH, CCH₂CH(NH₂)CH₂CH₃, CCH₂CH(NH₂)CH₂CH₂OH, CCH₂CH(NH₂)CH₂CH₂F, CCH₂CH(NH₂)CH₂CHF₂, or CCH₂CH(NH₂)CH₂CH(CH₃)₂.

8. (canceled)

9. (canceled)

10. (canceled)

11. (canceled)

12. (canceled)

13. (canceled)

14. The compound of claim 1, or pharmaceutically acceptable salt thereof, wherein X⁴ is N.

15. The compound of claim 1, or pharmaceutically acceptable salt thereof, wherein X⁴ is CH.

16. The compound of claim 1, or pharmaceutically acceptable salt thereof, wherein X⁴ is CR²⁴, and wherein R²⁴ is selected from the group consisting of halo, CN, and substituted or unsubstituted C₁₋₆ alkyl.

17. The compound of claim 1, or pharmaceutically acceptable salt thereof, wherein X⁴ is CCl, CBr, CF, CCN, or CCH₃.

18. (canceled)

19. (canceled)

20. (canceled)

21. (canceled)

22. The compound of claim 1, or pharmaceutically acceptable salt thereof, wherein X⁴ is C(cyclopropyl).

23. The compound of claim 1, or pharmaceutically acceptable salt thereof, wherein X⁸ is N.

24. The compound of claim 1, or pharmaceutically acceptable salt thereof, wherein X⁸ is CR²⁸.

25. A compound, or a pharmaceutically acceptable salt thereof, wherein the compound is selected from Table 1.

26. A pharmaceutical composition comprising the compound of claim 1, or a pharmaceutically acceptable salt thereof, and a pharmaceutically acceptable excipient or carrier.

27. (canceled)

28. A method of modulating splicing of a Ataxin3 (ATXN3) pre-mRNA, comprising contacting the compound of claim 1, or a pharmaceutically acceptable salt thereof, to the ATXN3 pre-mRNA with a splice site sequence or cells comprising the ATXN3 pre-mRNA, wherein the compound binds to the ATXN3 pre-mRNA and modulates splicing of the ATXN3 pre-mRNA in a cell of a subject to produce a spliced product of the ATXN3 pre-mRNA.

29. (canceled)
